(19) United States
(12) Patent Application Publication
(10) Pub. No.: US 2025/0136994 A1
(43) Pub. Date: May 1, 2025

(54) COMPOSITIONS AND METHODS TO RESTORE PATERNAL UBE3A GENE EXPRESSION IN HUMAN ANGELMAN SYNDROME

(71) Applicant: University of Connecticut, Farmington, CT (US)

(72) Inventors: Stormy Chamberlain, Oxford, CT (US); Noelle Germain, Watertown, CT (US)

(21) Appl. No.: 19/006,482

(22) Filed: Dec. 31, 2024

Related U.S. Application Data
(62) Division of application No. 17/271,982, filed on Feb. 26, 2021, now Pat. No. 12,221,609.
(60) Provisional application No. 62/734,435, filed on Sep. 21, 2018.

(51) Int. Cl. C12N 15/113; C12N 15/86

(52) U.S. Cl. CPC C12N 15/1137; C12N 15/86; C12N 2310/121; C12N 2310/122; C12N 2310/128; C12N 2310/141; C12N 2310/531; C12N 2740/15042; C12N 2740/15043; C12N 2750/14142; C12N 2750/14143

(57) ABSTRACT

The present invention relates to compositions and methods for activating expression from the paternally-inherited allele of UBE3A in human Angelman's Syndrome neurons using viral vector delivery of short hairpin RNAs, ribozymes, and/or microRNAs.

### CROSS-REFERENCE TO RELATED APPLICATIONS

This application is a Divisional Application of U.S. application Ser. No. 17/271,982, filed Feb. 26, 2021, which is a National Stage Application of International Application No. PCT/US19/52272, filed Sep. 20, 2019, which claims priority to U.S. Provisional Patent Application No. 62/734,435, filed on Sep. 21, 2018, the entire contents of which are fully incorporated herein by reference.

### REFERENCE TO SEQUENCE LISTING SUBMITTED VIA EFS-WEB

The application contains a Sequence Listing which has been submitted electronically in .XML format and is hereby incorporated by reference in its entirety. Said. XML copy, created on Sep. 23, 2024, is named “2262-91 PCT US DIV .xml” and is 845,827 bytes in size. The sequence listing contained in this .XML file is part of the specification and is hereby incorporated by reference herein in its entirety.

### TECHNICAL FIELD

The present disclosure relates to compositions and methods for activating expression from the paternally-inherited allele of UBE3A in human Angleman Syndrome neurons using short hairpin RNAs, ribozymes, and/or microRNAs.

### BACKGROUND OF THE INVENTION

Angelman syndrome (AS) is a neurodevelopmental disorder affecting ˜1/15,000 individuals. Individuals with AS have developmental delay, severe cognitive impairment, ataxic gait, frequent seizures, short attention span, absent speech, and characteristic happy demeanor. Neurons derived from induced pluripotent stem cells (iPSC) from AS patients exhibit a depolarized resting membrane potential, delayed action potential development, and reduced spontaneous synaptic activity. AS affects a relatively large patient population; a contact registry with >3,000 patients has been established and ˜250 new diagnoses of AS are made each year. Individuals with AS require life-long care.

AS is caused by loss of function from the maternal copy of UBE3A, a gene encoding an E3 ubiquitin ligase. This loss of function mutation can be caused by any type of gene mutation in the maternal allele. UBE3A is expressed exclusively from the maternal allele in neurons. All individuals with AS have a normal paternal UBE3A allele that is epigenetically silenced by a long, non-coding RNA, called UBE3A antisense transcript (UBE3A-ATS). Reactivation of the paternal allele has been shown to restore UBE3A protein expression and alleviate behavioral deficits in an AS mouse model. The restoration of UBE3A expression in humans is expected to ameliorate the disease, especially if it is restored in infants.

There is no cure for Angelman syndrome, however, there are two approaches being pursued by pharmaceutical companies and academic labs to cure this disorder. The first is the use of antisense oligonucleotides (ASOs) to cut UBE3A-ATS and activate paternal UBE3A. The second approach is AAV-mediated gene therapy to introduce the UBE3A gene back to the patient. The ASOs mentioned above do not cross the blood-brain barrier and require repeated injections into the spinal cord for life. The gene therapy to introduce the UBE3A gene back into neurons lacks the ability to regulate UBE3A mRNA and protein levels and requires the choice of a single protein isoform from three total, where the function of the individual isoforms remains uncertain. This may lead to overexpression of UBE3A, which may also contribute to another related disorder, Dup15q syndrome, and the absence of an important protein isoform.

### BRIEF SUMMARY OF THE INVENTION

The present invention provides a novel treatment approach for AS through gene therapy by inhibiting the silencing of paternal UBE3A and enabling the expression of paternal UBE3A from its natve regulatory elements. Increased expression of UBE3A in neurons and may ameliorate the effects of UBE3A-ATS on neurons listed above. All three protein isoforms may express using the endogenous regulatory elements that normally control their levels. Thus, overexpression is prevented due to the use of native regulatory elements. This approach may improve AS symptoms through a single treatment and thereby avoid the repeated spinal cord injections required for ASOs.

In one aspect, the invention provides a polynucleotide comprising a first nucleotide sequence encoding a short hairpin RNA (shRNA), ribozyme, or microRNA, wherein the shRNA, ribozyme, or microRNA is capable of inhibiting the silencing of paternal UBE3A.

In another aspect, the invention provides an expression vector comprising the polynucleotide having the first nucleotide sequence encoding the shRNA, ribozyme, or microRNA, wherein the shRNA, ribozyme, or microRNA is capable of inhibiting the silencing of paternal UBE3A; and a promoter.

In another aspect, the invention provides a viral particle comprising the polynucleotide having the first nucleotide sequence encoding the shRNA, ribozyme, or microRNA, wherein the shRNA, ribozyme, or microRNA is capable of inhibiting the silencing of paternal UBE3A.

In another aspect, the invention provides a pharmaceutical composition comprising the polynucleotide having the first nucleotide sequence encoding the shRNA, ribozyme, or microRNA, wherein the shRNA, ribozyme, or microRNA is capable of inhibiting the silencing of paternal UBE3A, or the viral particle comprising the polynucleotide, and a pharmaceutically acceptable carrier.

In another aspect, the invention provides a method of treating Angelman's syndrome comprising administering to a patient in need thereof, a therapeutically effective amount of the viral particle comprising the polynucleotide having the first nucleotide sequence encoding the shRNA, ribozyme, or microRNA, wherein the shRNA, ribozyme, or microRNA is capable of inhibiting the silencing of paternal UBE3A, or a pharmaceutical composition thereof.

In another aspect, the invention provides a method of activating or increasing expression of paternal UBE3A gene expression comprising administering to a patient in need thereof, a therapeutically effective amount of the viral particle comprising the polynucleotide having the first nucleotide sequence encoding the shRNA, ribozyme, or microRNA, wherein the shRNA, ribozyme, or microRNA is capable of inhibiting the silencing of paternal UBE3A, or a pharmaceutical composition thereof.

In another aspect, the invention provides a method of inhibiting the silencing of paternal UBE3A gene by the RNA antisense transcript encoded by SEQ ID NO: 1, comprising administering to a patient in need thereof, a therapeutically effective amount of the viral particle comprising the polynucleotide having the first nucleotide sequence encoding the short hairpin RNA (shRNA), ribozyme, or microRNA, wherein the shRNA, ribozyme, or microRNA is capable of inhibiting the silencing of paternal UBE3A, or a pharmaceutical composition thereof.

In another aspect, the invention provides a shRNA, ribozyme, or microRNA encoded by the polynucleotide described herein and capable of inhibiting the silencing of paternal UBE3A.

### BRIEF DESCRIPTION OF THE DRAWINGS

FIG. 1 shows chromosomal mutations in Angelman Syndrome.

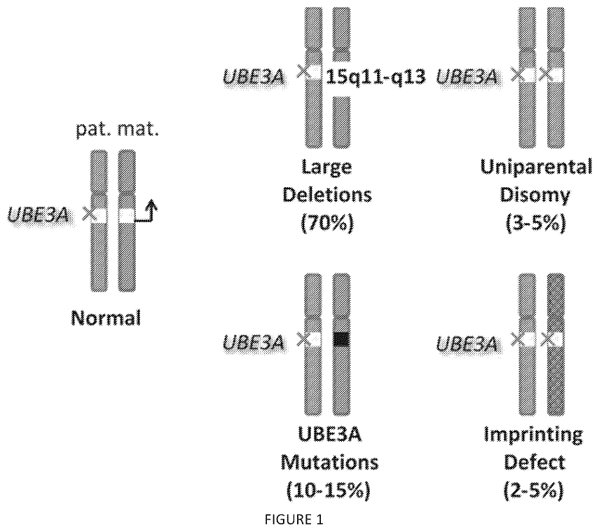

FIG. 2 shows a diagram of paternal UBE3A gene.

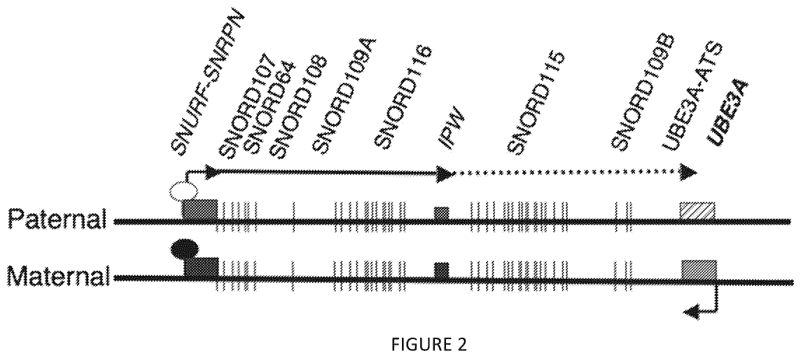

FIG. 3A shows Hammerhead ribozymes cut UBE3A-ATS in vitro. Hammerhead ribozyme and UBE3A-ATS DNA were transcribed in vitro using PCR. The resulting Ribozyme and UBE3A-ATS RNA were incubated in vitro (no cells and no vectors) and the cutting was visualized on agarose gel.

FIG. 3B shows Hammerhead ribozymes cut UBE3A-ATS in human AS iPSC-derived neurons.

FIG. 4 shows shRNAs knockdown UBE3A-ATS and activate paternal UBE3A. RT-qPCR was performed in AS iPSC-derived neurons transduced with lentivirus to express shRNAs targeting UBE3A-ATS. One of three shRNAs knocked down both UBE3A-ATS and activated paternal UBE3A.

FIG. 5 shows AS neurons transduced with AAV9 particles carrying 551 shRNA 2. The AAV vector also carries EGFP, allowing for green fluorescence upon transduction of a neuron. These neurons were transduced with 1×10⁵ viral genomes per cell.

FIG. 6 shows the plasmid map for pAV-U6-GFP, the AAV vector 551 used to clone shRNA2.

### DETAILED DESCRIPTION OF THE INVENTION

### UBE3A

UBE3A is a gene which encodes the E3 ubiquitin ligase. The genomic coordinates for UBE3A are hg19 chr15:25,582,381-25,684,175 on the minus strand. There are three normal isoforms of UBE3A: Isoform 1 (accession number X98032); Isoform 2 (accession number X98031); and isoform 3 (Accession number X98033). In neurons, UBE3A is expressed exclusively from the maternal allele. The paternal UBE3A allele is epigenetically silenced by a long, non-coding RNA, called UBE3A antisense transcript (UBE3A-ATS) encoded by SEQ ID NO: 1. The genomic coordinates for UBE3A-ATS are hg19 chr15:25,223,730-25,664,609 on the plus strand. The following genomic coordinates are of particular interest: hg19 chr15:25,522,751-25,591,391 on the plus strand.

The compositions and methods of the invention are drawn to targeting the UBE3A antisense transcript (UBE3A-ATS) to unsilence the paternal UBE3A allele. Effective inhibition of UBE3A-ATS by short hairpin RNAs (shRNA), ribozymes, or microRNAs may result in a reduction in UBE3A-ATS expression levels and a concomitant increase in the expression levels of the paternal UBE3A allele.

In several aspects of the invention, the compositions and methods herein relate to the treatment or prevention of AS. In certain aspects of any of the foregoing embodiments drawn to a method of treating a patient or human in need, the patient or human in need has AS or is at risk for developing AS. As used herein, the term “patient in need” includes any mammal in need of these methods of treatment or prophylaxis, including particularly humans. The subject may be male or female. In certain aspects, the patient in need, having AS, treated according to the methods provided herein may show an improvement in anxiety, learning, balance, motor function, and/or seizures, or the method may return the neuronal resting membrane potential to about-70 mV, ameliorate the action potential development delay, increase spontaneous synaptic activity, and may ameliorate additional alterations in the neuronal phenotype relating to rheobase, action potential characteristics (e.g. shape), membrane current, synaptic potentials, ion channel conductance, etc.

shRNA, Ribozyme, microRNA, and Coding Sequences

According to an aspect of the invention, a polynucleotide comprises a first nucleotide sequence encoding a short hairpin RNA (shRNA), a ribozyme, or a microRNA that results in decreased expression of the UBE3A-ATS sequence SEQ ID NO: 1. For example, a portion of the shRNA, ribozyme, or microRNA may be complementary to the RNA sequence encoded by SEQ ID NO: 1 or a sequence contained therein.

The shRNA, ribozyme, and microRNA are RNA polynucleotides encoded by a first nucleotide sequence. The polynucleotide comprising the first nucleotide sequence may be a DNA polynucleotide suitable for cloning into an appropriate vector (e.g., a plasmid) for culturing and subsequent production of viral particles. In turn, viral particles may contain the DNA polynucleotide with the nucleotide coding sequence in a form suitable for infection. Thus, the first nucleotide sequence may be a DNA sequence cloned into a plasmid for viral particle production or encapsulated in a viral particle. As retroviruses carry nucleotidecoding sequences in the form of RNA polynucleotides, retroviral particles (e.g., lentivirus) contain an RNA polynucleotide that comprises the first nucleotide sequence as a corresponding RNA sequence.

The first nucleotide sequence may encode the shRNA. For example, the first nucleotide sequence may be SEQ ID NO: 2. The first nucleotide sequence may also be a modified SEQ ID NO: 2 having the bold nucleotides in SEQ ID NO: 2 replaced by any of SEQ ID NOs: 9-508 and the italicized nucleotides in SEQ ID NO: 2 replaced by nucleotides complementary to those in SEQ ID NOs: 9-508.

The first nucleotide sequence may encode the ribozyme. For example, the first nucleotide sequence may be SEQ ID NO: 3. The first nucleotide sequence may be SEQ ID NO: 4.

The first nucleotide sequence may encode the microRNA. For example, the first nucleotide sequence may be SEQ ID NO: 8. The first nucleotide sequence may also be a modified SEQ ID NO: 8 having the bold nucleotides in SEQ ID NO: 8 replaced by any of SEQ ID NOs: 9-508 and the italicized nucleotides in SEQ ID NO: 8 replaced by nucleotides complementary to those in SEQ ID NOs: 9-508. The italicized nucleotides and the nucleotides complementary to those in SEQ ID NOs: 9-508 may be less than 100% complementary.

As used herein, “targets” means an operative RNA polynucleotide capable of undergoing hybridization to a nucleotide sequence through hydrogen bonding, such as to a nucleotide sequence transcribed from a nucleotide sequence within the larger genomic sequence of UBE3A-ATS. The hybridization of an operative RNA polynucleotide to a nucleotide sequence transcribed from a nucleotide sequence with the larger genomic sequence of UBE3A-ATS may result in the reduced expression of UBE3A-ATS levels in the presence of the operative RNA polynucleotide compared to the expression levels of UBE3A-ATS in the absence of the operative RNA polynucleotide. Preferably, the operative RNA polynucleotide comprises the nucleotide sequence of the shRNA, ribozyme, or microRNA that is complementary to the RNA sequence encoded within the larger genomic sequence of UBE3A-ATS. For example, the shRNA or microRNA contain nucleotide sequences complementary to the RNA sequences encoded by SEQ ID NO: 5 and SEQ ID NOs: 9-508 and the ribozymes contain nucleotide sequences complementary to the RNA sequences encoded by SEQ ID NO: 6 or SEQ ID NO: 7. The operative RNA polynucleotide thus refers to an operative portion of the shRNA ribozyme, or microRNA following assimilation of the shRNA, ribozyme, or microRNA into a target organism and processing into a functional state.

“Reduce expression” refers to a reduction or blockade of the expression or activity of UBE3A-ATS and does not necessarily indicate a total elimination of expression or activity. Mechanisms for reduced expression of the target include hybridization of an operative RNA polynucleotide with a target sequence or sequences transcribed from a sequence or sequences within the larger genomic UBE3A-ATS sequence (SEQ ID NO: 1), wherein the outcome or effect of the hybridization is either target degradation or target occupancy with concomitant stalling of the cellular machinery involving, for example, transcription or splicing.

Without wishing to be bound to a particular theory, the shRNA, ribozyme, and microRNA of the invention may inhibit the silencing of paternal UBE3A by: (1) cutting the RNA transcript encoded by SEQ ID NO: 1; (2) reducing steady-state levels (i.e., baseline levels at homeostasis) of the RNA transcript encoded by SEQ ID NO: 1; and (3) terminating transcription of SEQ ID NO: 1. For example, cutting and reduction of steady-state levels of the RNA transcript encoded by SEQ ID NO: 1 may occur via a mechanism involving a RNA-induced silencing complex (RISC). Both shRNA and microRNA may utilize RISC. Once the vector carrying the genomic material for the shRNA or microRNA is integrated into the host genome, the shRNA or microRNA genomic material is transcribed in the host into pri-microRNA. The pri-microRNA is processed by a ribonuclease, such as Drosha, into pre-shRNA or pre-microRNA, respectively, and exported from the nucleus. The pre-shRNA or pre-microRNA is processed by an endoribonuclease such as Dicer to form small interfering RNA (siRNA) or microRNA, respectively. For siRNA, the siRNA is loaded into the RISC where the sense strand is degraded and the antisense strand acts as a guide that directs RISC to the complementary sequence in the mRNA. For microRNA, a single strand from the microRNA is loaded into the RISC which acts as a guide that directs RISC to the complementary sequence in the mRNA. RISC cleaves the mRNA when the sequence has perfect complementary and represses translation of the mRNA when the sequence has imperfect complementary. In another example, cutting and reduction of steady-state levels of the RNA transcript encoded by SEQ ID NO: 1 may occur via a mechanism involving a ribozyme. Once the vector carrying the genomic material for the ribozyme is integrated into the host genome it is transcribed in the host into RNA. The RNA forms a secondary structure that has a catalytic domain and a region that is complementary to the target mRNA. When the ribozyme binds to the target mRNA the catalytic domain cleaves the target mRNA. Transcription of SEQ ID NO: 1 may be terminated by the torpedo mechanism wherein 5′-3′ and 3′-5′ exonucleases (e.g. XRN2) attach to the cleaved, uncapped end of the target RNA that is being transcribed. The 5′-3′ exonuclease catches the polymerase and disengages the polymerase from the DNA. Thus, the shRNA, ribozyme, and microRNA encoded by the first nucleic acid sequence may increase expression of paternal UBE3A by decreasing the steady-state levels of UBE3A-ATS RNA.

As used herein, the term “nucleic acid” refers to molecules composed of monomeric nucleotides. Examples of nucleic acids include ribonucleic acids (RNA), deoxyribonucleic acids (DNA), single-stranded nucleic acids, double-stranded nucleic acids, small interfering ribonucleic acids (siRNA), short hairpin RNAs (shRNAs), and microRNAs (miRNA). “Nucleotide” means a nucleoside having a phosphate group covalently linked to the sugar portion of the nucleoside. “Oligonucleotide” or “polynucleotide” means a polymer of linked nucleotides each of which can be modified or unmodified, independent one from another.

As used herein, a “short hairpin RNA (shRNA) “includes a conventional stem-loop shRNA, which forms a precursor microRNA (pre-miRNA). “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, a conventional shRNA (i.e., not a miR-451 shRNA mimic) forms a primary miRNA (pri-miRNA) or a structure very similar to a natural pri-miRNA. The pri-miRNA is subsequently processed by Drosha and its cofactors into pre-shRNA. Therefore, the term “shRNA” includes pri-miRNA (shRNA-mir) molecules and pre-shRNA molecules.

A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). It is known in the art that the loop portion is at least 4 nucleotides long, preferably 6 nucleotides long (e.g. the underlined sequence in SEQ ID NO: 2). The terms “hairpin” and “fold-back” structures are also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches.

In some embodiments, shRNAs useful in this invention include, without limitation, modified shRNAs, including shRNAs with enhanced stability in vivo. Modified shRNAs include molecules containing nucleotide analogues, including those molecules having additions, deletions, and/or substitutions in the nucleobase, sugar, or backbone; and molecules that are cross-linked or otherwise chemically modified. The modified nucleotide(s) may be within portions of the shRNA molecule, or throughout it. For instance, the shRNA molecule may be modified, or contain modified nucleic acids in regions at its 5′ end, its 3′ end, or both, and/or within the guide strand, passenger strand, or both, and/or within nucleotides that overhang the 5′ end, the 3′ end, or both. (See Crooke, U.S. Pat. Nos. 6,107,094 and 5,898,031; Elmen et al., U.S. Publication Nos. 2008/0249039 and 2007/0191294; Manoharan et al., U.S. Publication No. 2008/0213891; MacLachlan et al., U.S. Publication No. 2007/0135372; and Rana, U.S. Publication No. 2005/0020521; all of which are hereby incorporated by reference.)

In the present invention, shRNAs comprise a nucleotide sequence complementary to a RNA nucleotide sequence transcribed from within the full genomic UBE3A-ATS sequence (SEQ ID NO: 1) and inhibit the silencing of paternal UBE3A by UBE3A-ATS. In further embodiments, shRNAs comprise a nucleotide sequence complementary to RNA sequences encoded by SEQ ID NOs: 9-508. In a more particular embodiment, a shRNA comprises a nucleotide sequence complementary to a RNA sequence encoded by SEQ ID NO: 5. In a particular embodiment the shRNA is encoded by the nucleotide sequence of SEQ ID NO: 2. In embodiments of the present invention, the nucleotide sequence comprised in the shRNA and complementary to the RNA nucleotide sequence transcribed from the UBE3A-ATS gene is 17-21 nucleotides in length. The complementary nucleotides may be contiguous or may be interspersed with non-complementary nucleotides. In some embodiments, the complementary nucleotide sequence is 21 nucleotides in length as indicated by the bold sequence in SEQ ID NO: 2. The shRNA may comprise a nucleotide sequence wherein 17, 18, 19, 20, or 21 nucleotides are complementary to the nucleotides in SEQ ID NOs: 5 or 9-508. The 17, 18, 19, 20, or 21 complementary nucleotides may be contiguous or may be interspersed with non-complementary nucleotides. The overall length of the shRNA, including the loop may be 40-50 nucleotides in length, preferably 44-48 nucleotides, more preferably 48 nucleotides.

As used herein, the term “ribozyme” refers to an RNA molecule that acts like an enzyme or a molecule composed of a protein comprising the RNA molecule and is also called a RNA enzyme or catalytic RNA. It has been found that ribozymes catalyze chemical reactions with RNA molecules with a definite tertiary structure and have catalytic or autocatalytic properties. Some ribozymes cleave themselves or other RNA molecules to inhibit activity, and other ribozymes catalyze the aminotransferase activity of ribosomes. Such ribozymes may include hammerhead ribozymes, VS ribozymes, hairpin ribozymes, etc. In the present invention the ribozymes comprise a nucleotide sequence complementary to a RNA nucleotide sequence transcribed from within the full genomic UBE3A-ATS sequence (SEQ ID NO: 1) and inhibit the silencing of paternal UBE3A by UBE3A-ATS. In further embodiments, ribozymes comprise a nucleotide sequence complementary to RNA sequences encoded by SEQ ID NO: 6 and/or SEQ ID NO: 7. In further embodiments the ribozyme is encoded by the nucleotide sequence of SEQ ID NO: 3 or 4. In SEQ ID NOs: 3 and 4, the bold sequences are complementary to SEQ ID NOs: 6 or 7 and the underlined sequences signify the catalytic region of the ribozyme.

As used herein the terms “microRNA”, “miRNA” and “miR” are used synonymously to refer to an about 17-21 nucleotide (nt) long, non-coding RNAs derived from endogenous genes. MicroRNAs are processed from longer (ca 75 nt) hairpin-like precursors termed pre-miRNAs. MicroRNAs assemble in ribonucleoprotein complexes termed miRNPs and recognize their targets by antisense complementarity. If the microRNAs match 100% their target, i.e. the complementarity is complete, the target mRNA is cleaved, and the miRNA acts like a siRNA. If the match is incomplete, i.e. the complementarity is partial, then the translation of the target mRNA is blocked. In embodiments of the present invention, the nucleotide sequence comprised in the microRNA and complementary to the RNA nucleotide sequence transcribed from the UBE3A-ATS gene is 17-21 nucleotides in length. The complementary nucleotides may be contiguous or may be interspersed with non-complementary nucleotides. In some embodiments, the complementary nucleotide sequence is 21 nucleotides in length as indicated by the bold sequence in SEQ ID NO: 8. The microRNA may comprise a nucleotide sequence wherein 17, 18, 19, 20, or 21 nucleotides are complementary to the nucleotides in SEQ ID NOs: 5 or 9-508. The 17, 18, 19, 20, or 21 complementary nucleotides may be contiguous or may be interspersed with non-complementary nucleotides. The overall length of the precursor microRNA, including the loop may be 50-1000 nucleotides in length, preferably 59-67 nucleotides, more preferably 67 nucleotides.

In the present invention the microRNAs are designed to target one or more nucleotide sequences transcribed from one or more nucleotide sequences within the full genomic UBE3A-ATS sequence (SEQ ID NO: 1) and inhibit the silencing of paternal UBE3A by UBE3A-ATS. In further embodiments, microRNAs are designed to target one or more sequences transcribed from one or more sequences selected from SEQ ID NOs: 9-508. In a more particular embodiment microRNAs target a sequence transcribed from SEQ ID NO: 5. In further embodiments the microRNA is encoded by the nucleotide sequence of SEQ ID NO: 8 where the bold sequence is complementary to SEQ ID NOs: 5 or 9-508.

Methods of determining whether a sequence is specifically hybridizable to a target nucleic acid are well known in the art. In certain embodiments, the shRNA, ribozyme, or microRNA polynucleotides provided herein comprise a nucleic acid sequence specifically hybridizable with a RNA sequence transcribed from the UBE3A-ATSSEQ ID NO: 1.

The shRNA or microRNA may comprise an RNA polynucleotide containing a region of 17-21 linked nucleotides complementary to the RNA target sequence, wherein the RNA polynucleotide region is at least 85% complementary over its entire length to an equal length region of a UBE3A-ATS RNA nucleic acid sequence. In certain aspects, the RNA polynucleotide region is at least 90%, at least 95%, or 100% complementary over its entire length to an equal length region of a UBE3A-ATS RNA nucleic acid sequence.

The shRNA or microRNA may comprise a nucleotide sequence at least 85% complementary to, and of equal length as, a RNA sequence encoded by SEQ ID NO: 5 or any of SEQ ID NOs: 9-508. The shRNA or microRNA may comprise a nucleotide sequence at least 90% complementary to, and of equal length as, a RNA sequence encoded by SEQ ID NO: 5 or any of SEQ ID NOs: 9-508. The shRNA or microRNA may comprise a nucleotide at least 95% complementary to, and of equal length as, a RNA sequence encoded by SEQ ID NO: 5 or any of SEQ ID NOs: 9-508. The shRNA or microRNA may comprise a nucleotide sequence 100% complementary to, and of equal length as, a RNA sequence encoded by SEQ ID NO: 5 or any of SEQ ID NOs: 9-508.

The ribozyme may comprise an RNA polynucleotide containing two regions of linked nucleotides complementary to the RNA target sequence, separated by a catalytic region, wherein the overall non-catalytic region of the RNA polynucleotide is at least 85% complementary over its entire length to an equal length region of a UBE3A-ATS RNA nucleotide sequence. In certain aspects, the overall non-catalytic region of the RNA polynucleotide is at least 90%, at least 95%, or 100% complementary over its entire length to an equal length region of a UBE3A-ATS RNA nucleotide sequence. In embodiments of the present invention, the nucleotide sequence comprised in the ribozyme and complementary to the RNA nucleotide sequence transcribed from the UBE3A-ATS gene is 17-21 nucleotides in length. The complementary nucleotides may be may be interspersed with non-complementary nucleotides. In some embodiments, the complementary nucleotide sequence is 21 nucleotides in length as indicated by the bold sequence in SEQ ID NOs: 3-4. The ribozyme may comprise a nucleotide sequence wherein 17, 18, 19, 20, or 21 nucleotides are complementary to the nucleotides in SEQ ID NOs: 6-7. The 17, 18, 19, 20, or 21 complementary nucleotides may be interspersed with non-complementary nucleotides. The overall length of the shRNA, including the catalytic loop may be 50-150 nucleotides in length, preferably 57-65 nucleotides, more preferably 59 nucleotides.

The ribozyme may comprise a nucleotide sequence at least 85% complementary to, and of equal length as, a RNA sequence encoded by SEQ ID NO: 6 or 7. The ribozyme may comprise a nucleotide sequence at least 90% complementary to, and of equal length as, a RNA sequence encoded by SEQ ID NO: 6 or 7. The ribozyme may comprise a nucleotide sequence at least 95% complementary to, and of equal length as, a RNA sequence encoded by SEQ ID NO: 6 or 7. The ribozyme may comprise a nucleotide sequence 100% complementary to, and of equal length as, a RNA sequence encoded by SEQ ID NO: 6 or 7.

In certain aspects, the shRNA, ribozyme, or microRNA, is a single-stranded RNA polynucleotide. In several aspects, the RNA polynucleotide is a modified RNA polynucleotide. A percent complementarity is used herein in the conventional sense to refer to base pairing between adenine and thymine, adenine and uracil (RNA), and guanine and cytosine.

Non-complementary nucleobases between a shRNA, ribozyme, or microRNA and an UBE3A-ATS nucleotide sequence may be tolerated provided that the shRNA, ribozyme, or microRNA remains able to specifically hybridize to a UBE3A-ATS nucleotide sequence. Moreover, an shRNA, ribozyme, or microRNA may hybridize over one or more segments of a UBE3A-ATS nucleotide sequence such that intervening or adjacent segments are not involved in the hybridization event (e.g., a loop structure, mismatch or hairpin structure).

In certain embodiments, the shRNA, ribozyme, or microRNA provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a UBE3A-ATS RNA nucleotide sequence, a UBE3A-ATS region, UBE3A-ATS segment, or specified portion thereof. Percent complementarity of a shRNA, ribozyme, or microRNA with an UBE3A-ATS nucleotide sequence can be determined using routine methods.

For example, a shRNA, ribozyme or microRNA in which 18 of 20 nucleobases of the shRNA, ribozyme or microRNA are complementary to a UBE3A-ATS region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, a shRNA, ribozyme, or microRNA which is 18 nucleobases in length having four non-complementary nucleobases which are flanked by two regions of complete complementarity with the target nucleotide sequence would have 77.8% overall complementarity with the target nucleotide sequence and would thus fall within the scope of the present invention. Percent complementarity of a shRNA, ribozyme, or microRNA with a region of a UBE3A-ATS nucleotide sequence can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the shRNA, ribozyme, or microRNA provided herein, or specified portions thereof, are fully complementary (i.e. 100% complementary) to a UBE3A-ATS nucleotide sequence, or specified portion of the transcription product of SEQ ID NO: 1 thereof. For example, a shRNA, ribozyme, or microRNA may be fully complementary to a UBE3A-ATS nucleotide sequence, or a region, or a segment or sequence thereof. As used herein, “fully complementary” means each nucleobase of a shRNA, ribozyme, or microRNA is capable of precise base pairing with the corresponding RNA nucleobases transcribed from a UBE3A-ATS nucleotide sequence.

An effective concentration or dose of the shRNA, ribozyme, or microRNA may inhibit the silencing of paternal UBE3A by UBE3A-ATS by at least 20%, at least 30%, at least 40%, at least 50%, at least 60%, at least 70%, at least 80%, at least 90%, or at least 100%.

An effective concentration or dose of the shRNA, ribozyme, or microRNA may terminate transcription of UBE3A-ATS by at least 20%, at least 30%, at least 40%, at least 50%, at least 60%, at least 70%, at least 80%, at least 90%, or at least 100%.

An effective concentration or dose of the shRNA, ribozyme, or microRNA may reduce steady-state levels of UBE3A-ATS by at least 20%, at least 30%, at least 40%, at least 50%, at least 60%, at least 70%, at least 80%, at least 90%, or at least 100%.

An effective concentration or dose of the shRNA, ribozyme, or microRNA may cut UBE3A-ATS and reduce it by at least 20%, at least 30%, at least 40%, at least 50%, at least 60%, at least 70%, at least 80%, at least 90%, or at least 100%.

An effective concentration or dose of the shRNA, ribozyme, or microRNA may reduce expression of UBE3A-ATS by at least 20%, at least 30%, at least 40%, at least 50%, at least 60%, at least 70%, at least 80%, at least 90%, or at least 100% and induce expression of paternal UBE3A by at least 20%, at least 30%, at least 40%, at least 50%, at least 60%, at least 70%, at least 80%, at least 90%, or at least 100%.

As used herein, the terms “UBE3A-ATS” and “Ube3A-ATS” can be used interchangeably without capitalization of their spelling referring to any particular species or ortholog. “UBE3A” and “Ube3A” can be used interchangeably without capitalization of their spelling referring to any particular species or ortholog. Additionally, “UBE3A”, “UBE3A”, “Ube3A”, and “Ube3A” can be used interchangeably without italicization referring to nucleic acid or protein unless specifically indicated to the contrary.

### Viral Vector

A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, plasmids that contain a viral genome, viruses, or artificial chromosomes. The term “vector” includes cloning and expression vectors, as well as viral vectors and integrating vectors.

As will be evident to one of skill in the art, the term “viral vector” is widely used to refer to a nucleic acid molecule (e.g., a transfer plasmid) that includes viral nucleic acid elements that typically facilitate transfer of the nucleic acid molecule to a cell or to a viral particle that mediates nucleic acid sequence transfer and/or integration of the nucleic acid sequence into the genome of a cell.

Viral vectors contain structural and/or functional genetic elements that are primarily derived from a virus. The viral vector is desirably non-toxic, non-immunogenic, easy to produce, and efficient in protecting and delivering DNA or RNA into the target cells. According to the compositions and methods of the inventions a viral vector may contain the DNA that encodes the shRNA, ribozyme, and/or microRNA of the invention. In particular embodiments, the viral vector is a lentiviral vector or an adeno-associated viral (AAV) vector.

As used herein, the term “lentivirus” refers to a group (or genus) of complex retroviruses. Illustrative lentiviruses include, but are not limited to: HIV (human immunodeficiency virus; including HIV type 1, and HIV type 2); visna-macdi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). Lentivirus will transduce dividing cells and postmitotic cells.

The term “lentiviral vector” refers to a viral vector (e.g., viral plasmid) containing structural and functional genetic elements, or portions thereof, including long terminal repeats (LTRs) that are primarily derived from a lentivirus. A lentiviral vector is a hybrid vector (e.g., in the form of a transfer plasmid) comprising retroviral e.g., lentiviral, sequences for reverse transcription, replication, integration and/or packaging of nucleic acid sequences (e.g., coding sequences). The term “retroviral vector” refers to a viral vector (e.g., transfer plasmid) containing structural and functional genetic elements, or portions thereof, that are primarily derived from a retrovirus.

Adenoviral vectors are designed to be administered directly to a living subject. Unlike retroviral vectors, most of the adenoviral vector genomes do not integrate into the chromosome of the host cell. Instead, genes introduced into cells using adenoviral vectors are maintained in the nucleus as an extrachromosomal element (episome) that persists for an extended period of time. Adenoviral vectors will transduce dividing and non-dividing cells in many different tissues in vivo including airway epithelial cells, endothelial cells, hepatocytes, and various tumors (Trapnell, 1993; Chuah et al., 2003).

The term adeno-associated virus (AAV) refers to a small ssDNA virus which infects humans and some other primate species, not known to cause disease, and causes only a very mild immune response. AAV can infect both dividing and non-dividing cells and may incorporate its genome into that of the host cell. These features make AAV a very attractive candidate for creating viral vectors for gene therapy, although the cloning capacity of the vector is relatively limited. In a preferred embodiment of the invention, the vector used is derived from adeno-associated virus (i.e. AAV vector). More than 30 naturally occurring serotypes of AAV are available. Many natural variants in the AAV capsid exist, allowing identification and use of an AAV with properties specifically suited for specific types of target cells. AAV viruses may be engineered by conventional molecular biology techniques, making it possible to optimize these particles for cell specific delivery of shRNA, ribozyme, or microRNA DNA sequences, for minimizing immunogenicity, for tuning stability and particle lifetime, for efficient degradation, for accurate delivery to the nucleus, etc.

An “expression vector” is a vector that includes a regulatory region. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.). An expression vector may be a viral expression vector derived from a particular virus.

The vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of pLK0.1 puro, SV40 and, plasmids such as RP4; phage DNAs, e.g., the numerous derivatives of phage 1, e.g., NM989, and other phage DNA, e.g., M13 and filamentous single stranded phage DNA, vectors useful in eukaryotic cells, such as vectors useful in insect or mammalian cells, vectors derived from combinations of plasmids and phage DNAs, such as plasmids that have been modified to employ phage DNA or other expression control sequences.

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34:167-171 (2003). A large variety of such vectors are known in the art and are generally available.

A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation).

In some embodiments, the viral vector used in the invention will be used at a concentration of at least 10⁵ viral genomes per cell.

The selection of appropriate promoters can readily be accomplished. An example of a suitable promoter is an RNA polymerase II or III promoter, such as the U6 promoter. Other suitable promoters which may be used for gene expression include, but are not limited to, the 763-base-pair cytomegalovirus (CMV) promoter, the Rous sarcoma virus (RSV) (Davis, et al., Hum Gene Ther 4:151 (1993)), the SV40 early promoter region, the herpes thymidine kinase promoter, the regulatory sequences of the metallothionein (MMT) gene, PGK (phosphoglycerol kinase) promoter, alkaline phosphatase promoter; and the animal transcriptional control regions, which exhibit tissue specificity and have been utilized in transgenic animals: myelin basic protein gene control region which is active in oligodendrocyte cells in the brain, and gonadotropic releasing hormone gene control region which is active in the hypothalamus. Certain proteins can be expressed using their native promoter. Other elements that can enhance expression can also be included such as an enhancer or a system that results in high levels of expression such as a tat gene and tar element. This cassette can then be inserted into a vector, e.g., a plasmid vector such as, pLK0.1, pUC19, pUC118, pBR322, or other known plasmid vectors. Sec, Sambrook, et al., Molecular Cloning: A Laboratory Manual, Cold Spring Harbor Laboratory press, (1989). The plasmid vector may also include a selectable marker such as the β-lactamase gene for ampicillin resistance, provided that the marker polypeptide does not adversely affect the metabolism of the organism being treated. The cassette can also be bound to a nucleic acid binding moiety in a synthetic delivery system, such as the system disclosed in WO 95/22618.

Coding sequences for shRNA, ribozymes, and microRNA can be cloned into viral vectors using any suitable genetic engineering technique well known in the art, including, without limitation, the standard techniques of PCR, polynucleotide synthesis, restriction endonuclease digestion, ligation, transformation, plasmid purification, and DNA sequencing, as described in Sambrook et al. (Molecular Cloning: A Laboratory Manual. Cold Spring Harbor Laboratory Press, N.Y. (1989)), Coffin et al. (Retroviruses. Cold Spring Harbor Laboratory Press, N.Y. (1997)) and “RNA Viruses: A Practical Approach” (Alan J. Cann, Ed., Oxford University Press, (2000)). In a preferred embodiment, the shRNA, ribozyme, and microRNA DNA sequences contain flanking sequences on the 5′ and 3′ ends that are complementary with sequences on the plasmid and/or vector that is cut by a restriction endonuclease. As is well known in the art, the flanking sequences depend on the restriction endonucleases used during the restriction digest of the plasmid and/or vector. Thus, one of skill in the art can select the flanking sequences on the 5′ and 3′ ends of the shRNA, ribozyme, and microRNA DNA sequences accordingly. In another preferred embodiment, the target sites can be cloned into vectors by nucleic acid fusion and exchange technologies currently known in the art, including, Gateway, PCR in fusion, Cre-lox P, and Creator.

In some embodiments, an expression vector comprises a promoter and a polynucleotide comprising a first nucleotide sequence encoding a short hairpin RNA (shRNA), ribozyme, or microRNA of the invention. Preferably, the promoter and the polynucleotide comprising the first nucleotide sequence are operably linked. Preferably, the promoter is a U6 promoter. The first nucleotide sequence included in the expression vector may be SEQ ID NO: 2. The first nucleotide sequence included in the expression vector may be SEQ ID NO: 3. The first nucleotide sequence included in the expression vector may be SEQ ID NO: 4. The first nucleotide sequence included in the expression vector may be SEQ ID NO: 8. The polynucleotide comprising the first nucleotide sequence in the expression vector is preferably a DNA polynucleotide. The first nucleotide sequence of the expression vector is preferably a DNA nucleotide sequence. The shRNA, ribozyme, or microRNA encoded by the first nucleotide sequence of the expression vector may be as described in any of the variations disclosed herein.

As discussed below, recombinant viral vectors are transfected into packaging cells or cell lines, along with elements required for the packaging of recombinant viral particles. Recombinant viral particles collected from transfected cell supernatant are used to infect target cells or organisms for the expression of shRNAs, ribozymes, or microRNA. The transduced cells or organisms are used for transient expression or selected for stable expression.

### Viral Particle

Viral particles are used to deliver coding nucleotide sequences for the shRNAs, ribozymes, and microRNAs which target UBE3A-ATS RNA. Viral particles will typically include various viral components and sometimes also host cell components in addition to nucleic acid(s). Nucleic acid sequences may be packaged into a viral particle that is capable of delivering the shRNA, ribozyme, and/or microRNA nucleic acid sequences into the target cells in the patient in need or human.

The viral particles may be produced by (a) introducing a viral expression vector into a suitable cell line; (b) culturing the cell line under suitable conditions so as to allow the production of the viral particle; (c) recovering the produced viral particle; and (d) optionally purifying the recovered infectious viral particle.

An expression vector containing the nucleotide sequence encoding the shRNA, ribozyme, or microRNA of the invention may be introduced into an appropriate cell line for propagation or expression using well-known techniques readily available to the person of ordinary skill in the art. These include, but are not limited to, microinjection of minute amounts of DNA into the nucleus of a cell (Capechi et al., 1980, Cell 22, 479-488), CaPO₄-mediated transfection (Chen and Okayama, 1987. Mol. Cell Biol. 7, 2745-2752), DEAE-dextran-mediated transfection, electroporation (Chu et al., 1987, Nucleic Acid Res. 15, 1311-1326), lipofection/liposome fusion (Feigner et al., 1987, Proc. Natl. Acad. Sci. USA 84, 7413-7417), particle bombardment (Yang et al., 1990. Proc. Natl. Acad. Sci. USA 87, 9568-9572), gene guns, transduction, infection (e.g. with an infective viral particle), and other techniques such as those found in Sambrook, et al. (Molecular Cloning: A Laboratory Manual, Cold Spring Harbor Laboratory, Cold Spring Harbor Laboratory Press, Cold Spring Harbor, N.Y., 2001).

When the vector of the invention is defective, the infectious particles are usually produced in a complementation cell line or via the use of a helper virus, which supplies in trans the non-functional viral genes. For example, suitable cell lines for complementing adenoviral vectors include the 293 cells (Graham et al., 1997, J. Gen. Virol. 36, 59-72) as well as the PER-C6 cells (Fallaux et al., 1998, Human Gene Ther. 9, 1909-1917) commonly used to complement the E1 function. Other cell lines have been engineered to complement doubly defective adenoviral vectors (Yeh et al., 1996. J. Virol. 70, 559-565; Krougliak and Graham, 1995, Human Gene Ther. 6, 1575-1586; Wang et al., 1995, Gene Ther. 2, 775-783; Lusky et al., 1998, J. Virol. 72, 2022-2033; WO94/28152 and WO97/04119). The infectious viral particles may be recovered from the culture supernatant but also from the cells after lysis and optionally are further purified according to standard techniques (chromatography, ultracentrifugation in a cesium chloride gradient as described for example in WO 96/27677, WO 98/00524, WO 98/22588, WO 98/26048, WO 00/40702, EP 1016700 and WO 00/50573).

The invention also relates to host cells which comprise the nucleic acid molecules, vectors, or infectious viral particles of the invention described herein. For the purpose of the invention, the term “host cell” should be understood broadly without any limitation concerning particular organization in tissue, organ, or isolated cells. Such cells may be of a unique type of cells or a group of different types of cells and encompass cultured cell lines, primary cells, and proliferative cells.

Host cells therefore include prokaryotic cells, lower eukaryotic cells such as yeast, and other eukaryotic cells such as insect cells, plant and higher eukaryotic cells, such as vertebrate cells and, with a special preference, mammalian (e.g. human or non-human) cells. Suitable mammalian cells include but are not limited to hematopoietic cells (totipotent, stem cells, leukocytes, lymphocytes, monocytes, macrophages, APC, dendritic cells, non-human cells and the like), pulmonary cells, tracheal cells, hepatic cells, epithelial cells, endothelial cells, muscle cells (e.g. skeletal muscle, cardiac muscle or smooth muscle) or fibroblasts. Preferred host cells include Escherichia coli, Bacillus, Listeria, Saccharomyces, BHK (baby hamster kidney) cells, MDCK cells (Madin-Darby canine kidney cell line), CRFK cells (Crandell feline kidney cell line). CV-1 cells (African monkey kidney cell line), COS (e.g., COS-7) cells, chinese hamster ovary (CHO) cells, mouse NIH/3T3 cells, Hela cells and Vero cells. Host cells also encompass complementing cells capable of complementing at least one defective function of a replication-defective vector of the invention (e.g. adenoviral vector) such as those cited above.

The host cell of the invention may be further encapsulated. Cell encapsulation technology has been previously described (Tresco et al., 1992, ASAJO J. 38, 17-23; Aebischer et al., 1996. Human Gene Ther. 7, 851-860). According to said specific embodiment, transfected or infected eukaryotic host cells are encapsulated with compounds which form a microporous membrane and said encapsulated cells may further be implanted in vivo. Capsules containing the cells of interest may be prepared employing hollow microporous membranes (e.g. Akzo Nobel Faser A G, Wuppertal, Germany; Deglon et al. 1996, Human Gene Ther. 7, 2135-2146) having a molecular weight cutoff appropriate to permit the free passage of proteins and nutrients between the capsule interior and exterior, while preventing the contact of transplanted cells with host cells

Viral particles suitable for use in the invention include AAV particles and lentiviral particles. AAV particles carry the coding sequences for shRNA, ribozymes, and microRNAs in the form of genomic DNA. Lentiviral particles, on the other hand, belong to the class of retroviruses and carry the coding sequences for shRNA, ribozymes, and microRNAs in the form of RNA.

Recombinantly engineered viral particles such as AAV particles, artificial AAV particles, self-complementary AAV particles, and lentiviral particles that contain the DNA (or RNA in the case of lentiviral particles) encoding the shRNAs, ribozymes, and/or microRNAs targeting UBE3A-ATS RNA may be delivered to target cells to inhibit the silencing of UBE3A by UBE3A-ATS. The use of AAVs is a common mode of delivery of DNA as it is relatively non-toxic, provides efficient gene transfer, and can be easily optimized for specific purposes. In one embodiment of the invention, the selected AAV serotype has native neurotropisms. In further embodiments of the invention, the AAV serotype is AAV9 or AAV10.

A suitable recombinant AAV is generated by culturing a host cell which contains a nucleotide sequence encoding an AAV serotype capsid protein, or fragment thereof, as defined herein; a functional rep gene; a minigene composed of, at a minimum, AAV inverted terminal repeats (ITRs) and a coding nucleotide sequence; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

Unless otherwise specified, the AAV ITRs, and other selected AAV components described herein, may be readily selected from among any AAV serotype, including, without limitation, AAV1, AAV2, AAV3, AAV5, AAV6, AAV7, AAV8, AAV9, AAV10 or other known and unknown AAV serotypes. These ITRs or other AAV components may be readily isolated using techniques available to those of skill in the art from an AAV serotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

The minigene, rep sequences, cap sequences, and helper functions required for producing the rAAV of the invention may be delivered to the packaging host cell in the form of any genetic element which transfers the sequences carried thereon. The selected genetic element may be delivered by any suitable method. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, 1993 J. Viral., 70:520-532 and U.S. Pat. No. 5,478,745, among others. These publications are incorporated by reference herein.

Selection of these and other common vector and regulatory elements are conventional and many such sequences are available. See, e.g., Sambrook et al, and references cited therein at, for example, pages 3.18-3.26 and 16.17-16.27 and Ausubel et al., Current Protocols in Molecular Biology, John Wiley & Sons, New York, 1989). Of course, not all vectors and expression control sequences will function equally well to express all of the transgenes of this invention. However, one of skill in the art may make a selection among these, and other, expression control sequences without departing from the scope of this invention.

### Pharmaceutical Compositions and Therapeutic Treatment

The viral particles comprising the desired coding sequences for the shRNA, ribozyme, and/or microRNA can be formulated for administration to a patient or human in need by any means suitable for administration. Such formulation involves the use of a pharmaceutically and/or physiologically acceptable vehicle or carrier, particularly one suitable for administration to the brain, e.g., by subcranial or spinal injection. Further, more than one shRNA, ribozyme, and/or microRNA, or any combination thereof, may be administered in a combination treatment. In a combination treatment, the different shRNA, ribozyme, and/or microRNA may be administered simultaneously, separately, sequentially, and in any order.

Suitably, the pharmaceutical composition of the invention comprises a carrier and/or diluent appropriate for its delivering by injection to a human or animal organism. Such carrier and/or diluent is non-toxic at the dosage and concentration employed. It is selected from those usually employed to formulate compositions for parental administration in either unit dosage or multi-dose form or for direct infusion by continuous or periodic infusion. It is preferably isotonic, hypotonic or weakly hypertonic and has a relatively low ionic strength, such as provided by sugars, polyalcohols and isotonic saline solutions. Representative examples include sterile water, physiological saline (e.g. sodium chloride), bacteriostatic water, Ringer's solution, glucose or saccharose solutions, Hank's solution, and other aqueous physiologically balanced salt solutions (see for example the most current edition of Remington: The Science and Practice of Pharmacy, A. Gennaro, Lippincott, Williams & Wilkins). The pH of the composition of the invention is suitably adjusted and buffered in order to be appropriate for use in humans or animals, preferably at a physiological or slightly basic pH (between about pH 8 to about pH 9, with a special preference for pH 8.5). Suitable buffers include phosphate buffer (e.g. PBS), bicarbonate buffer and/or Tris buffer. A particularly preferred composition is formulated in IM saccharose, 150 mM NaCl, 1 mM MgCl₂, 54 mg/l Tween 80, 10 mM Tris pH 8.5. Another preferred composition is formulated in 10 mg/ml mannitol, 1 mg/ml HSA, 20 mM Tris, pH 7.2, and 150 mM NaCl. These compositions are stable at −70° C. for at least six months.

The composition of the invention may be in various forms, e.g. in solid (e.g. powder, lyophilized form), or liquid (e.g. aqueous). In the case of solid compositions, the preferred methods of preparation are vacuum drying and freeze-drying which yields a powder of the active agent plus any additional desired ingredient from a previously sterile-filtered solution thereof. Such solutions can, if desired, be stored in a sterile ampoule ready for reconstitution by the addition of sterile water for ready injection.

Nebulized or aerosolized formulations also form part of this invention. Methods of intranasal administration are well known in the art, including the administration of a droplet, spray, or dry powdered form of the composition into the nasopharynx of the individual to be treated from a pressured container or dispenser which contains a suitable propellant, e.g., a gas such as carbon dioxide, or a nebulizer (see for example WO 95/11664). Enteric formulations such as gastroresistant capsules and granules for oral administration, suppositories for rectal or vaginal administration also form part of this invention. For non-parental administration, the compositions can also include absorption enhancers which increase the pore size of the mucosal membrane. Such absorption enhancers include sodium deoxycholate, sodium glycocholate, dimethyl-beta-cyclodextrin, lauroyl-1-lysophosphatidylcholine and other substances having structural similarities to the phospholipid domains of the mucosal membrane.

The composition can also contain other pharmaceutically acceptable excipients for providing desirable pharmaceutical or pharmacodynamic properties, including for example modifying or maintaining the pH, osmolarity, viscosity, clarity, color, sterility, stability, rate of dissolution of the formulation, modifying or maintaining release or absorption into an the human or animal organism. For example, polymers such as polyethylene glycol may be used to obtain desirable properties of solubility, stability, half-life and other pharmaceutically advantageous properties (Davis et al., 1978, Enzyme Eng. 4, 169-173; Burnham et al., 1994, Am. J. Hosp. Pharm. 51, 210-218). Representative examples of stabilizing components include polysorbate 80, L-arginine, polyvinylpyrrolidone, trehalose, and combinations thereof. Other stabilizing components especially suitable in plasmid-based compositions include hyaluronidase (which is thought to destabilize the extra cellular matrix of the host cells as described in WO 98/53853), chloroquine, protic compounds such as propylene glycol, polyethylene glycol, glycerol, ethanol, 1-methyl L-2-pyrrolidone or derivatives thereof, aprotic compounds such as dimethylsulfoxide (DMSO), diethylsulfoxide, di-n-propylsulfoxide, dimethylsulfone, sulfolane, dimethyl-formamide, dimethylacetamide, tetramethylurea, acetonitrile (see EP 890 362), nuclease inhibitors such as actin G (WO 99/56784) and cationic salts such as magnesium (Mg²⁺) (EP 998 945) and lithium (Li⁺) (WO 01/47563) and any of their derivatives. The amount of cationic salt in the composition of the invention preferably ranges from about 0.1 mM to about 100 mM, and still more preferably from about 0.1 mM to about 10 mM. Viscosity enhancing agents include sodium carboxymethylcellulose, sorbitol, and dextran. The composition can also contain substances known in the art to promote penetration or transport across the blood barrier or membrane of a particular organ (e.g. antibody to transferrin receptor; Friden et al., 1993, Science 259, 373-377). A gel complex of poly-lysine and lactose (Midoux et al., 1993, Nucleic Acid Res. 21, 871-878) or poloxamer 407 (Pastore, 1994, Circulation 90, 1-517) may be used to facilitate administration in arterial cells.

The viral particles and pharmaceutical compositions may be administered to patients in therapeutically effective amounts. As used herein, the term “therapeutically effective amount” refers to an amount sufficient to realize a desired biological effect. For example, a therapeutically effective amount for treating Angelman's syndrome is an amount sufficient to ameliorate one or more symptoms of Angelman's syndrome, as described herein (e.g. developmental delay, severe cognitive impairment, ataxic gait, frequent seizures, short attention span, absent speech, and characteristic happy demeanor). Further, AS iPSC-derived neurons exhibit a depolarized resting membrane potential, delayed action potential development, and reduced spontaneous synaptic activity. Thus, a therapeutically effective amount for treating AS may return the neuronal resting membrane potential to about −70 mV, ameliorate the action potential development delay, increase spontaneous synaptic activity, or ameliorate additional alterations in the neuronal phenotype relating to rheobase, action potential characteristics (e.g. shape), membrane current, synaptic potentials, ion channel conductance, etc.

The appropriate dosage may vary depending upon known factors such as the pharmacodynamic characteristics of the particular active agent, age, health, and weight of the host organism; the condition(s) to be treated, nature and extent of symptoms, kind of concurrent treatment, frequency of treatment, the need for prevention or therapy and/or the effect desired. The dosage will also be calculated dependent upon the particular route of administration selected. Further refinement of the calculations necessary to determine the appropriate dosage for treatment is routinely made by a practitioner, in the light of the relevant circumstances. For general guidance, a composition based on viral particles may be formulated in the form of doses of at least 10⁵ viral genomes per cell. The titer may be determined by conventional techniques. A composition based on vector plasmids may be formulated in the form of doses of between 1 μg to 100 mg, advantageously between 10 μg and 10 mg and preferably between 100 μg and 1 mg. The administration may take place in a single dose or a dose repeated one or several times after a certain time interval.

The composition of the invention can be enclosed in ampoules, disposable syringes or multiple dose vials made of glass or plastic. In all cases, the composition must be sterile and should be fluid to the extent that easy syringability exists. It must be stable under the conditions of manufacture and storage and must be preserved against the contaminating action of microorganisms such as bacteria and fungi. Sterile injectable solutions can be prepared by incorporating the active agent (e.g., infectious particles) in the required amount with one or a combination of ingredients enumerated above, followed by filtered sterilization.

The viral particles and pharmaceutical compositions of the present invention are preferably administered by a parenteral route including intravenous, intraarterial, subcutaneous, intraperitoneal or intramuscular injection or infusion, intrathecal or intracranial, e.g., intracerebral or intraventricular, administration. In one embodiment, the viral particles or pharmaceutical compositions are administered intracerebral or intracerebroventricular. In another embodiment the viral particles or pharmaceutical compositions are administered intrathecal.

In certain embodiments of the methods of this invention, the viral particles and pharmaceutical composition described above are administered to the subject by subcranial injection into the brain or into the spinal cord of the patient or human in need. In a particular embodiment of the invention the use of subcranial administration into the brain results in the administration of the encoding nucleotide sequences of the invention directly to brain cells, including glia and neurons. As used herein, the term “neuron” refers to any cell in, or associated with the function of the brain. The term may refer to any one the types of neurons, including unipolar, bipolar, multipolar and pseudo-unipolar.

### EXAMPLES

### General Methods

Human Embryonic Stem Cell (hESC) Culture and Neural Differentiation

hESCs were cultured on irradiated mouse embryonic fibroblasts and fed daily with hESC media (DMEM/F12 containing knockout serum replacement, L-glutamine+β-mercaptoethanol, non-essential amino acids, and basic fibroblast growth factor). hESCs were cultured in at 37° C. in a humid incubator at 5% CO2. Cells were manually passaged every 5-7 days.

hESCs were differentiated into neurons using a modified version of the monolayer protocol. Neural induction was begun 2 days after passaging by culturing cells in N2B27 medium (Neurobasal medium, 1% N2, 2% B27, 2 mM L-glutamine, 0.5% penicillin/streptomycin, 1% insulin-transferrin-selenium). N2B27 medium was supplemented with fresh Noggin (500 ng/mL) for the first 10 days of differentiation. Neural rosettes were manually passaged onto poly-D-lysine and laminin coated plates using the Stem Pro EZ passage tool approximately 14 days after beginning neural induction. Neural progenitors were replated at a high density around 3 weeks of differentiation, switched to neural differentiation medium (NDM) around 4 weeks of differentiation, then plated sparsely for terminal differentiation at around 5 weeks. NDM consisted of neurobasal medium, 1% B27, 2 mM L-glutamine, 0.5% pen-strep, non-essential amino acids, 1 μM ascorbic acid, 200 μM cyclic AMP, 10 ng/mL brain-derived neurotrophic factor, and 10 ng/ml glial-derived neurotrophic factor. Neurons were maintained in culture until 10 to 17 weeks of differentiation. The protocols for hESC maintenance and neuronal differentiation have been described previously.

Generation of shRNA and Ribozyme Vectors

shRNAs and ribozymes were generated by annealing two complementary polynucleotides with the desired sequences. Specifically, the polynucleotides to generate shRNAs were comprised of the specific 21-nucleotide sequence to be targeted and its reverse complement, separated by a loop sequence of CTCGAG, and with a 5′ flank sequence of CCGG and a 3′ flank sequence of TTTTTG added for cloning into the plasmid vector. The polynucleotides to generate ribozymes were comprised of the specific 41 nucleotide sequence along with the same 5′ and 3′ flanking sequences for plasmid cloning. The annealed polynucleotides were ligated into the pLK0.1 puro vector (Stewart et al RNA 2003 April; 9 (4): 493-501). The shRNA and ribozyme sequences are under the control of the U6 promoter. The resulting plasmid was subjected to Sanger sequencing to confirm correct insertion of shRNA sequences.

### Lentiviral Production and Transduction of Neurons

Lentiviral particles were made by transfecting 293 FT cells with 2^{nd} generation packaging systems using Lipofectamine 3000. Virus was concentrated using the Lenti-X Concentrator Kit (Clontech) and viral titer calculated using the qPCR Lentivirus Titration Kit (abm). 10-week old neurons were plated on laminin-coated plastic dishes at a density of 1.3 cells per cm² and transduced with 10 viral particles per cell. Neurons were harvested for RNA seven days after lentiviral transduction.

### Quantitative RT-PCR

cDNA was synthesized using the High-Capacity cDNA Reverse Transcription Kit (Thermo Fisher Scientific) according to manufacturer's instructions. Quantitative RT-PCR was performed using Taqman Gene Expression Assays for each intended target and Mastermix (Thermo Fisher Scientific) on the Step One Plus (Thermo Fisher Scientific). Reactions were performed in technical duplicates, with GAPDH Endogenous Control Taqman Assay used as the housekeeping gene for normalization. Gene expression was quantified using the ΔΔCₜ method.

### AAV Production and Transduction of Neurons

551 shRNA 2 as well as a scrambled shRNA control were cloned into the pAV-U6-GFP vector and packaged into viral particles by a commercial vendor (Vigene). Neurons were transduced with the 551 shRNA 2 AAV9 particles using at least 1×10⁵ viral genomes per cell. After 48 hours, neurons were imaged to visualize transduced neurons.

### Example 1

To test the cutting abilities of ribozymes in vitro, ribozyme and target RNA sequences were in vitro transcribed and tested under controlled conditions. Specifically, primers including a T7 sequence were used to amplify the genomic sequence of the intended target. The PCR product was cleaned and concentrated before being used in an in vitro transcription assay with T7 polymerase. The DNAs were used in an in vitro transcription reaction with T7 polymerase. RNA was column purified using MEGAclear kit (Ambion). Ribozyme RNAs were prepared by annealing two complementary ribozyme polynucleotides. They were then end-filled with T4 polymerase and cleaned up and concentrated. The ribozymes were diluted to an appropriate concentration (5pmol/μL) in ddH20 as a working stock. The ribozymes and target RNAs were combined in an in vitro cutting assay for 30 minutes at 37 degrees C. and visualized with ethidium bromide on a 2% agarose gel.

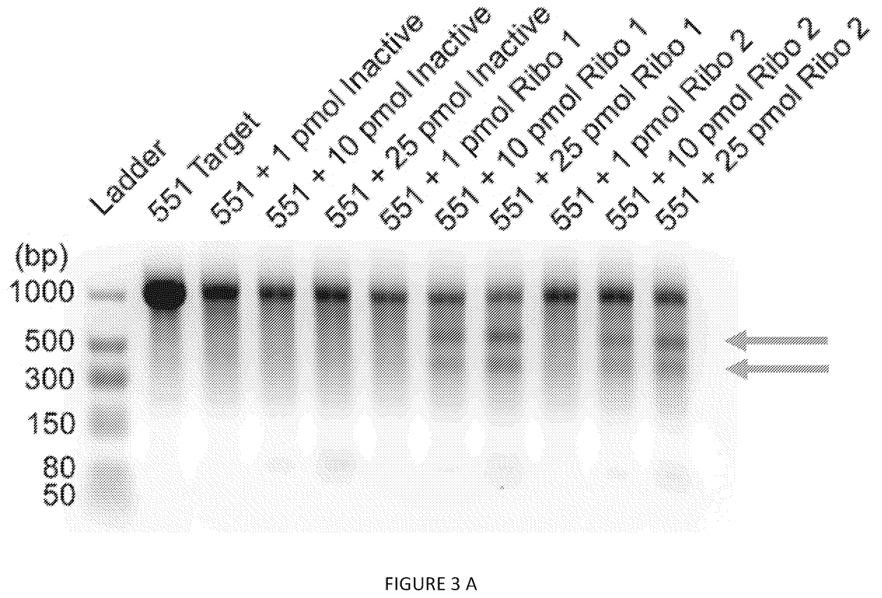

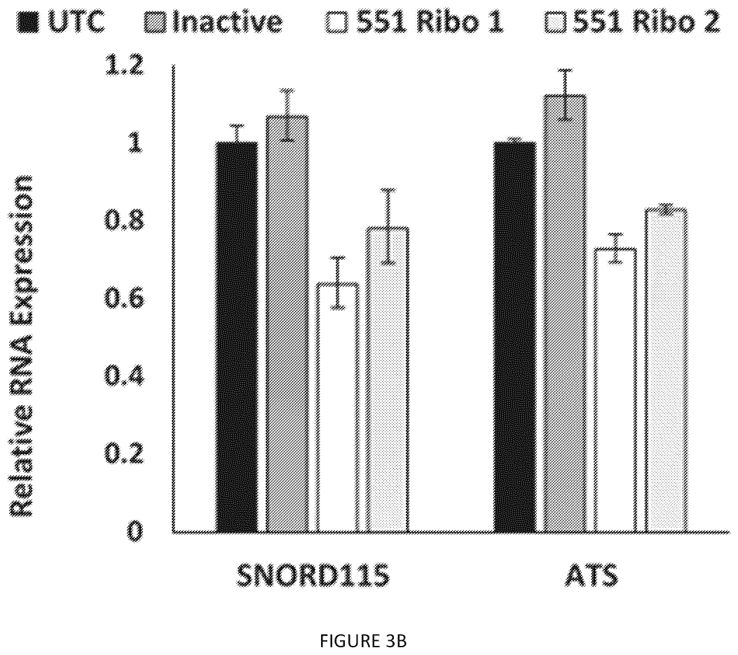

Hammerhead ribozymes, which are catalytic RNA molecules that cause hydrolysis of phosphodiester bonds in RNAs were tested first. These ribozymes cleave quickly and independently of other cellular machinery, and have been tested in clinical trials for HIV and certain tumors. Four hammerhead ribozymes were designed to cut near sequences targeted by functional ASOs. In vitro cutting assays revealed that the ribozymes, were active and can cut the UBE3A-ATS sequence (FIG. 3A). The most active two ribozymes, 551 Ribo 1 (SEQ ID NO: 3) and 551 Ribo 2 (SEQ ID NO: 4), were encoded by lentiviral vectors which were packaged into lentiviral particles and used to transduce mature AS iPSC-derived neurons. UBE3A-ATS levels were reduced by ˜40% (FIG. 3B).

### Example 2

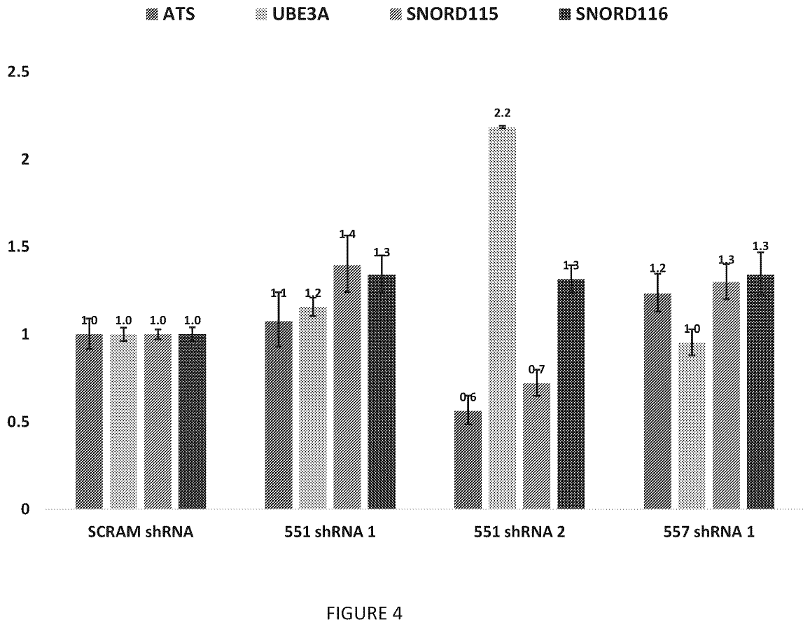

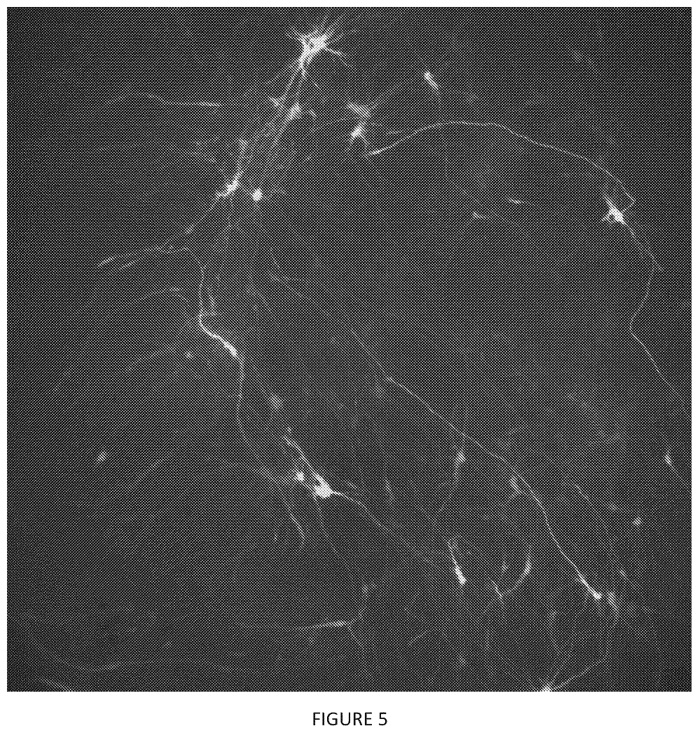

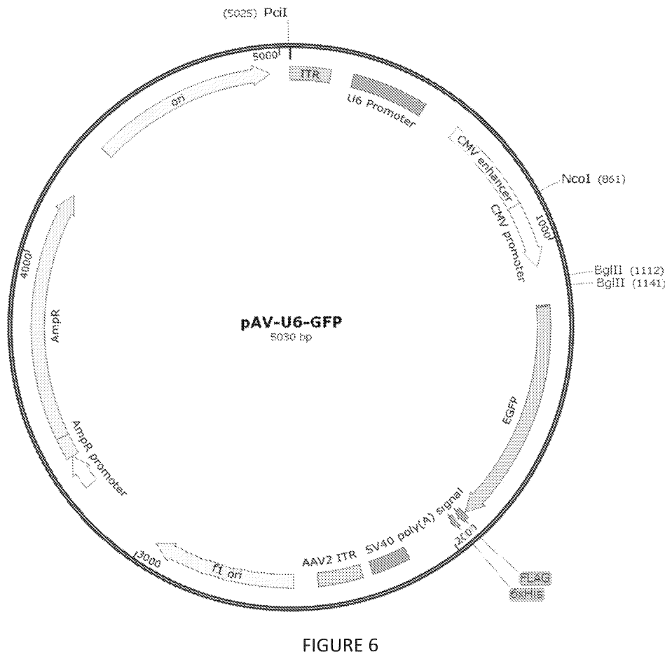

shRNAs were developed which targeted similar sequences in the UBE3A-ATS gene. shRNAs enlist the RNA interference pathway to knockdown RNAs. Four shRNAs were designed by cloning them into lentiviral vectors which were packaged into lentiviral particles and used to transduce mature AS iPSC-derived neurons. 551 shRNA2 (SEQ ID NO: 2) achieved 40% knockdown of UBE3A-ATS, and a 2.2-fold activation of paternal UBE3A (FIG. 4). These studies have been repeated by cloning the same shRNAs into adeno associated viral (AAV) vectors and packaging them into AAV9 particles (FIGS. 5 and 6).

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

Sequences

SEQ ID NO: 1
Human UBE3A-ATS genomic sequence.
TGAGATGACCTAAACAACTGTGGAGAATCATTGATATATTTCCTTTTTTCACTGTTCATGTTGG
GTGAAAATAATCTTGTAGTGAAATTCACATGTTCTAAATATTGTTTTTTTACATCTTTATCTGG
CACATTCATAACATAGATGTTTCTATACATATTAGTACTGTAATCATACCATATATTATTCTGT
TACCCCACTTACTCCTTAAACTTTTAGTTAATTAAAGAGTTTTTATAAAGTCCCCCAATAGATT
TTTTTTTTTTGAGACATAGTCTCACTCTGTTGCCCAGGCTGGAGTGCAGTGGCGTGATTTTGGC
TCACTGCAACCTCCCCATCCTGGGTTCAAGCAATTCTCCTGCCTCAGCCTGCCAAGTAGCTGGG
ATTACAGGTGCCTGCCACCACGCCCGACTAATTTTTGTATTTTCAGTAGTGACAGGGTTTCACC
ATCTTGGCCAGGCTGGTCTTGAACTCCTGACCTCGTGATCCACCCGCCTTGGCCTCTCAGAGTG
CTAGGATTACAGGCTTGAGCCACTGCACCCGGCCAGATTTTAATCTAATTTTATTAGAACAATT
CAGTCATATGTTTTTTCATGCTATGTATATGAGAGTTCCATTATTCAGATACTAAACAAATGTC
TACTGTACATTTACTGTTCTCACTGATGATGCATTAGATAACCATGCACAAAATAAGCCTGGCT
GTGGAAACGCTTATTTGTTGGGAGGGTGCTTGTTTGGATCGATGATGAGAATAATTGTCTGAGG
ATGCTGAGGGACTCATTCCAGATGTCAATCTGAGGTCCAGATGTGCGGCCCTCCAATAGGACAA
ATAAGACTCTCAGAGCCTGGCTCTATTTGGGGATCCCTCAGTGACAACATAGTACCCCTGTGAG
CGTGCCTTTTCTATCTCTTCGAAGAGGGCAGTGGCATCCTGTCTTATGAGTCAGTGTGCACTTT
AGTGTGCCTAGTGACCCAAGACTTGCTTTAATTGTAGATAGATACTTACATATAGGAAATATTT
CTTAAGTAACAAATGAAAAACTTTAGAAGATTGAATTAAGGGTCAAGCAACTGTGATATGTCTG
AAAATCTCATTAGTGTTGTGCTGAAAGAAGGAAATATGGCATGCCTCTATTAAATAATGACAGT
GGAACCAAGTTTATTGCTTTGTTATTTTTACTGTGGAGTATTTTCTAAGATTATTTTTGCTTTT
TTTTTCTTTCATGTTTTGCTGAGATAGAAGGCCTGGAATCTGATCCTCCACTTCAGAGAACAGG
GGTGAGTAGCTAAGCCATTATCTTTTGAAATTCATATGTCATGTGCTCTTTGCTAGGTCTTTAG
GTCGTTTTGTACATCTTTTCAGAAGCTTATTGGAGGACATTTTCATGATATGTCCTTTTCCTCA
TTGAGACCCTCACCATGTCACCTACACTATTGAATCCTTATCATTTCTCTTTTAATTTTAACTC
TCTTTTGCTTTTATGGAAAAATGTAGAATTTAAGAGAATTTTTGGCAATTTCATATTGGATCAA
AATGTATTGTAGTGAAATCCAGGTGTGCCAAAATATTAACAGATTTTCCCCATCTGTTTAATTA
TTGGGGTTTCAGAATAGAGACTCCATGGTTCATAATATCTTTGTGGTCATACTACATTATATTT
CTGCTTCTAATTTAATTATTAAATATTGACTTGAATTAGTCTTTTCCTCATTGTTGCAACAAGG
TAAGTTATATAGGAAATTTTCTTCTCTTGATGGCATGTCTGAGATAATCATAGATATAAGACAC
CTGGCTGGTTTCTAGAATGCATGTAATTTTTATTTCTTGATCTGTGTGTTGAGTACTTGCTGTG
ATTGCATCATGCAAATACATTGAGCTTTACAATTTTAGTGTATGCACTTTTCTACATGTATATT
ATTCTTCGATAGAAAGTAAAAAAAACTTATCGAACTAGTCAAAATATTGGTTAATACATAAAAA
AAGTCTCAAGTAGATTGTGTATTACATGGTGCTTGTTGATTGATGCCCTCATAATAGATCAAGT
GGGTTCTCTCTTTAGCACAGGGCTTTTTAGCAAATCATGTCATGAGTAGTTACTCAAGTATTTT
TATTTTAACACATTTATATTTTTTCTATGTATATTCTTAAATTCTCTTATACTTTTTTCTCTGT
TATAAAAACATGCTGAACAATCTCAAGTCTTAAGGATTGCAGTATTGTCCCCACATATTCATGT
ATTTTGGTACTCAATTCTTTATACTTTCTTTGACAGATCACTTGAACTGGCACATGTCTCTTGT
TTTGCAGAGAGGGAATTAATGTGATACCTTCATGCTTTTCTATTCTATGTGCTACATAATTGAA
TATACAAGCAAATATAGTTGTTAAGATTTAGTGTGATTATTTCTACACCACATGCAAAGAAGTT
TCTCATAGATCTTAATAGAGGCCCACATGCATTGTACAGTTTAGAATTTGGGGAAATATTGATG
AAGTTGGGTAAAGTATAAAGCCAAAAGTCAGAACAGTGAACTCCTTGCTTAAGGATTTCCTTGG
AGATTACTTAGTCAATACACAACTGATAAATTTAAGTGCTTTTCACCTTTTGAGTTCTCGACAT
ACTAAAGCTAAAATGTGTTTCAACTTTTAATCCTGCTTCCCTGATTTTCCCTTTTTTAGTCTGA
GATCAAAGAGTTTCAGCCATAAATTACTGCCAAGAGTAATCACTTCATTTTAAGAAAGCTTAAC
AATATAGAAGAATATAAAATTATTTATGACAGATGTATTTTTAACCTTTTCCCCATGCTTTCCA
GAGGAAATATGTTTAATCATCTGCCCTATATTAGGGAAAAACTTTCTATGCTAATACAAGTATC
TATCAATCCATTTATCTTTCTATATAAGATGTATTGATCATAACCAATTAACTTTACTGTAAAT
GAGCTTTAGATTTGACATTTTGGTAGTAATATATGTTGTACAATCTCCTGAGGTCCTATAGGTC
TTGAGGTCTCTATGTCAAAAACTATAGATGTGCCAGTGTCCTCAGTGAATGTGAAGGACACCAC
ATTTTCCTTAGCCATTTCTTGTTTTCAGAATAATGGTTATCAACATTTTGCTACTGCAAGATAC
CATACATTTATAATCGGAATATGCCAGTTTTTATGCACTCATGCCTCTGTTTCTGTAGAGCATT
CCCAGAATGAGTAATGCTTGAAAATTAGGTCCATGTGATTTCTTTAATGAGTTATAGTCAAATC
ATGAAATATTCAGGTTACCATTATTTCAGTAATGTATTAGAATGTCAAGGTAAAGTTATCTACA
TTGTATATATACACACAACATAGATATAATTTATACAACATCTATATTTATACAACAGATATAT
ATTTATGTATATATTTATACAACATAAAATATATTTTATTATTTAAACATAAAATATATTTTAT
TATTTAATATAGATTCTTAAGTGATAAATATGTTTAATATTATTAAAATAGGTTAAAATAGGTT
ATAGTTAGTACAGTGAAAATTGGCAGCCCTTTTACAAAACATATGCCATAACTATAGCATTTAT
CACTGACAGTCATACCAGGATAGTCTTTTATTTCCAATCACTTAAATATTCCTAATTGCAAAAG
AAATTTGAAGACTAAAATTCAGAAGTTTTGAAAGAGCCATTGCCTGGGTAAACTATACAGGTTT
CAGTTTTATTTATAATAATTATGAGGCCAGGCGCAGTGGCTCACACCTGTAATCCCAACACTTT
GGGAGGCCGAGGCGGGTGGATCACGAGGTCAGGAGATCGAGACCATCCTGGCTAACACGGTGAA
ACCCCATCTCTACTAAAAATACAAAAAATTAGCCGGGCGTGGTGGCGGGCGCCTGTAGTCCCAG
CTACTCGGGAGGCTGAGGCAGGAGAATGGCGTGAACCTGGTAGGCGGAGCTTGCAGTGAGCCGA
GATCGCGCCACTGCCCTCCAGCCTGGGAGACAGTGCGAGACTCCGTCTCAAAAAAAAAAATTAT
GTATATATTTATAAATTAATACTTAATAAATTAATAACTTGTGATAGGCAATGCAAAGATGACA
GTAAAAGGACAAAATTGATTAGATTGATAAAGTCCTGTTAACATGAAGAAATTGACCAGGATAC
CATCCACACTATAAAGTTAGAGAAATTTATCAGGACAATTCCCTAAAATACTCTTCTCAATTTT
AACATTGTAACAGGAATTTTTAAAATTTTGGTATTATGTGTGTTTCCTTCCAGATAATTTGAAC
AGATTCATATTTGGTATTTTTAAAAGCCATATCTTTGTCCTTAGTGCTGGCAATGTATTCTTGA
GAATGAACAAATAAGAGATACGTAAAAGCATAAGAGAAGGTATCAGGTTGAAGTAGTCAATCAG
TTATACAGAACACAAAGAATTTTATCTTGTATAATGTTTATATAGCTTTATAGAAGTGTGCTGA
AAGGGCTATAAAACATGGACATTATTATCTCATTGAAAGGTCCAATACGTACTGAAATACATGC
TTTTATTTTGAACCAACCACCCTATAAACGTTGTATGGCTTATTTAGATGAGAGCCCAGGTTGT
GTGTGTCTGTGTGTGTGTGTGTGTGTGTGTGTGTGTGTGTGTGTGTGTGTGTACCTGACAGGGA
AGCAAGAACATCGAGTTGCCAATGCACTCTGTCTATGGTTAGAATCATGCTGAAAACATGGCTC
CCCCAGTTCTGGAATGAGCCCACAGATCAAGCATTCCCCAAAGACATAGCAGGCTCAAATCCCT
GTGTACACAATATTTTATGATTATCTTATGTCAGTACTTTCAAAGTATACAGTTTGTGTGAAGA
CAAATCCAATGTCATTTTTCTTGGCTAGCCTATATGTGTGGTAAATCCATTATTTACTTACTTG
CTTCCTGAAAATTACAATTAGATTAACAAACTGCAGCAAAGTGGGCATGATGAGATAGAGATTG
AAGTGTAAGCTTATGTTAATGATGCCCTTGGTTTGGATAAACACATCTAAGAGAAAAATGGAAA
AACACACATGGCAGGGAAGCCTTGATAGAGCCAAAATATAGGATTGTATGTAGTAATGCAATCC
ATAGATGAGCATTTGGCAGTAATATTATTTTTCAGATATGGATAAAAATTGCTTAGGAGAGTAA
AGAGAGACAAAGTTGAAAGCAGGTTTATAGTAGGTGTTGTTTTAGTGTTGATCCCTTTTTGCTC
CAATAATCAAAGTGATAAATATTGAAAATTGATTCATGCAGCATTACTTACTCCATTCTAATTT
TTATATATGTCAAAAGTGCCATCTCCCAAACTGTGCTATCCCCTTCAGGAGAAGAGACTCTGCT
GAAGTTTATAAGGTTGACATATTGCCAGCTTCAATAATGTAAAGATGAAGTGTATACTGAATTC
TTAATGCAAATAACAACTCTATTGGAAAGTAACCCAGTTATAGAAGTGCTAATTTGTCAGGAGC
TGCCTTACCAAGATCATGATGAGTACAGTTATCTCAGGATTCTGAAAGATTGTTTTCCGATTTC
AACTAGTCTAGCTGAATGTTCCTTGATAGAAAGAGAGGACTTTTAGAATTGGTTCAATATGATG
ACCTCCTGAATTATCTCACATAGCCCGTTTGTACATGCCTTTCTTTTCTCTCAGAAAATGGCAC
TATCATAATAGCTTTCTTACACAGACTTCACCTTAGGGTTTTACATTAAGGGAGGGGTCTGGTG
TTTCATTTATTTTGAAGTATTTGTTGTTGATTGTGTACAGTGCTTGAGTAAAAAATTGAATATA
GAAACATCTAGAATATTTTTTTAAAGGATCAGTGTTTATAAAGTGAATTATTAGTGTCAATAAT
GTTGGGAAAGTTTTAAGAGAATATAGGAAACTTGAACATTACACAACTACAATGGGACCAAATT
GTGGGGTCTCATTATAGTTAATATTTATGTATTTTTTTCCAATTGATTTGTGTGCTTTTTTTCT
GCATGTTTTTGGCAGATAGAATGGCTATAACAAGTAACAGCATGTCAGGTAATAAAAATAAGCA
GAGCCCTATTCCTTTAAAAATCTTCACTGATGGGAGGGCCATAAAATAAGTCTTAATACATTTA
AAGAATTAAATTCATGTAAACCATGTTAATTTAATTCCACAATGATATTGAATTAGAAATAAGA
GGAATATCTCTTGAACATCTCCTAAATGTTTGGAAATTTAAATTAGCATTTCTGACCTATTTAT
TGGTTAAAAAAGATACAAAGAAAGGAAAATTGAAAAGTCTTTTGAACTGAATAAAAATAAAAAT
ATAGAATCTAAAACTTTATGGGATACTGACAAAACAGGATATAGGGAATAATTTATAGCACTGA
AATGCCTATATTAGAAAAGAAAAAAGGTTTTAAATCAGTAAATTTGTATTTTACCTTAAGAAAC
TTAGAAAAGAACAAATTAACCCAGACTTAAGTAAAATAAAGGCACTAATAAAGATAAGAGCAGA
AATCAATGAAATATAAAACAACAAAACACAGAGAAAAATTGAGAAAATTTAAAAATAGCCTAGT
GAGAAGATATTGATAAACTTGTAACCAGACCAATTTAAGAAAAAAAGTCAAAACACAAATACCA
ATATTTGAAAATGTAGGAGGGCAAATCATTACAGATTCTATGAATACTAAAATGATAATAAGGA
AAAATTATTTAAAAGGGGCATGTCAGCCAGGCATGGTGGCTTACCCCTGTAATCCCAGCACTTT
GGCAGGCCGAGGTGGGAGGATTGCTAGAGCTCAGGCATTCGAGACCAGCCTGGGCAACATGTTG
AAACCTTGTCTACACAAAAAGTACAAAAATTAGCTGGGTGTGGTGTTGCACACTTGTAGTCCCA
GTCACTTGGGAGGCTGAGGCGAGAGGATCACTTGAGCCCAGGAGGTTGAGGCTGCAGTGAGCCA
TGTTTGTACCACTGCCCTCCAGCCTGGGTGACAAAGTAAGACCCTATGTAAAAAAAAAAAAATG
TATGCCAACATTTTTCAATAACTTAAATGAAATGGAAAAATTCCTTGAAAGACACGAACTACAA
AAACTCAGTGAACAAGTAAATAACCTGAATAGCCCTGTATCAAGTAAATTGAATTTGTAGTTAA
AAGCCTTCCAACAGAGAAAACTTCAGGTACCTATAGCTTCATATGAAATGAAAAAAAAAATACC
AATCCTCTACAAGATTCCAGAACATTTAAAAGAAGGGAATATTTCCCAACTTATTCCATTTGGA
CAGCAATACCCAGGTAAGAAAAAGAGACACAGAAATTTAAAAAGAAGAATATACATTATTCCTT
AGGAACATAAATGCAAAGAAATCTAATCAAAATTTTGGCAAATGAAATGTAGAAATACTTTATG
ACCAAGTGAGAGTTACCCAAAGAATTTAAGGTTGGTTTTATATGTAAAGATCAACCAATATAGG
AAAATCACTTCTGGAAAGTCAGAGTAAGAAACTCCAAAAATCTACTCCTCCATAAAACCAATAA
CAGCCTTGATAGAAATAGTTGAAATTAATTTTCCAAAACTTTGGAAATTAACCAAAGGCTTACA
AAATTCCAGAGAACATTAATTCAAGAAAAATGGCTGAATCAGTAAGAACAGCCAGCTTTGTGGC
ATTTTAATATGACCCCTTCCCATGCTTTTCTCCCTAGTGCTGAGATAGTCTTAAAAATTAGCAG
GATAGCAACCACTGGAGAAGAAAGGTTTGGAAATTTCCCAAAAAGTTCCATCCCCATAGAATTA
TCACTATTTGACCTCTAAAGCCCAATCTATAGGATTTATATTCATTTGGACTGACTCAGAGCTC
ACTCAGTAAGGAAATCTCAATTTCAAGGTATTGGTCAAAAAGAATCCATGGCAATTGTTGACTA
TCACAACTGCCTGAAGTCTTGGTAACAGTTGGGATAAACAAGAAGCTGATCAAAAACTGAAAAC
TAAAATCTTGGGAATGAGATATCTACAGGATGCTTCAAAAAGCTTTGATACATTCCTGTTTATC
TAGAAAGCTACATGCAGGCTGGGTGCAGTGGCTCACACCTGTAATCCCAGCACTTTGGGAGGCC
GAGGCGTGCGGATCATGAGGTCAGGAGTTTGAGGCCAGCCTGACCAACATGGTCTCTACTAAAA
ACACAAAAATTAGCCAGGCGTGGTGGCGTGCATCTGTAATCCTAGCTACTCAGGAGGCTGAGAC
AGGAGAATCGCTTGAACCCGGGAGGCGGAGGTTGCAGTGAGCCAAGATTGTGCCACTGCACTCC
AGCCTGGGCGACAGAGCAAGGCTCTGTCTCAAAAAAAAAAAAAAAAAAAGCCACATGCATGTAA
TTGTTTACCTCTGGCTTTCCTTTTATGCTCTGGGCAAGCTAAGGAAGAGTTGTGAACTACCTAA
GTGCTGAATGGGAACCATAACACACACACACACACACACACACACACACACAGCACCTTAGTAA
AGGGTGAGAGGCATGTTAGTTAGAAGCATTAAAGGAAATCTCTTTCTAGTCATTATCTGTGCAC
TAACCTAACTGAGCAGAGACTTCAGTATCCACATACTACAGGGCATATAAACTTTACAGAATTA
GTCCAGGAAAATCATATCTAAAAAAAAAAAAAAGCAGTAACAAAAATAAACTCTGGGAAAGGGG
AGAATATGATTTAAAGAGTTGCCACATTATACATAATATGTCTAGTGTTCAACAAAAAATTACG
AGACATGCAAAGAAATAGAAAAATATGGCACAAAGAGGATAAGATGAAGTCAGTGAAACTATCC
TCGAGGAAGACCAGATGTTGGTCTTACTAGACACAGACATTGAACCAGCTATTAAAAATACGTA
CACAGAACTAAGAAAAACATGTCAAAAGGGTTAAAGAAAGGTATAAAAATAGTGTCTTACCAAA
TATAGACTACCAATAAAGAGATAGAAATTATAAGAAAAGACAACATGAAAAAATATAAAGCAAA
AAAATTAGACAATTGAAACAAGAGGGCCTCTATTCGCAGATTTGAGCAGGCAGAAGAAAGAATC
AGTGAACTTGAAGATATGTCAACTGAGATTATCCAGTCTGAGCAACAAAGGTGGGAAAAAATGA
AGAAAACTGAGCAACAAAGAACTGTAGAACAGCATCTCTCATACCAATGGATACATAAACTGGA
GCCCTAGAAGGATGAAAAAAGGAGAAGGAAAGAAAACTTCCCAAATTTTAAGAAAAACATTAAT
TTATATACCCGAGATGACCAATAAAATCCAATTAAGATAATCTCAAAGAGACCAACACCTATAC
ACATCATAGTCAGCGTGTCAAAAGACAAACATAAGGAGAGAATTCTTGAAGATAGTAAGAAAAA
AATTATTCATAACATACACACCATCCTCAATAAGTCTGACAATTGACTTCTCACTGTAAACCAT
GCAGGCTAAAAAGGCAATGTACATAACCAAAGTGATGAAATAAAAACCTTCAACCAATCATTCT
AGATCCAACAAAACTATTGTTCAAAAAGAAGAAATGAAGACATTCCTAAACAAAATCTCAGAGA
AATGTTCTCTATAAGACTTGTCCTAATAGAAATGCTAAAGGAACTCCTTCGGTCTGAAATAGAA
AGGCACTGGAGAGTAAATCAAATCCACGAGAAGAAATAAAGAGAACCAGTATAAGTAACTACAT
GTGTAAGTTTAAAACAAAGTATAAATTTATTTTGTTTGTAACATTTGTCTTTTCCTATTTGATT
TAAAATATAATCTCAATTATAAACTTGTGTTGATGGTATTATATAAAGATGTAATTTTGGGTTA
TAGCACCAAAATGGCAGAATAGGAATTTTCTGTAGGTGTTTCCCACATAAGTATCAATTTTGAC
AACCATCCATGGGCAAGAGTACCTTGTGGGAGTTCAGGAGTTGACAGTAAAACTTCAGCACACC
AGAGGAGTAAAGAAATCTAAGAATAGATTCATTGGAAAGGGTATAAACAGTTTCACTTTACCTG
CATCACCAACCCCCAAAAGTGGCACAGCTCAGTAACAAGAGCCCATTATTTCTTCCACAGAGGA
AAAGGAGAGTATAATAAGTAAGTGTCCAGTTTCTCAAGACATACAGGCCCCTGCCCAAGAGATC
CACTTTATTTTCATCTCACCCAGAATATTGAGGTGATCAGCAAGGTGGAGTGGTTGGGAGAGGG
TAAAAGCAGGAAAGAGAGATGGGGACTCAAACAGCAGCCCATACTTGGAACTGCCATAGATCCT
ACCAGTTACTTCATGGACTCCATCAGGAACCCACCTATAAGCCACAGGGGATGCATCCCTCCCA
TCTTGCCAACAAAACCCCGAATGCTCCAAATGCCTCACCCACTCTTTGGCTGGCTCCCAAGTGC
GCTCCTGTGAAAAGCGAGTGAGTATCTCTGCAGATGGCTTGCAAGCACATGTTGACAGCTGGCT
CCACTCTGTAGAACTGGAAAAAAGCTCACATACATGAGAATTTCAGGACACTACCCTAAAAAAA
AAAATGAGACTTCAGCACCTGGCCTGGCTTTATGCAACCTAGAGAAGGTGATATGATTTGGCTC
TGTGTCCCCACGTAAATCTCATGTCAAATTGTAATCCCCACGTGTTGGACAAGGGACTTGGTGG
GAGGTGATTGAATCATGCGGGTGGACTTCCCTCTTGCTGTTCTTGTGATAGAGTTGTTATGAGA
TCCAGTTATTTGAAAGTATGTAGCATGTCCCCCTTCACTCTCTTGCACTCCTGCTCCACCTTGG
TAAGACTTGCTTGCTACCCCTTTGCCTTCTGCCATGATTGTAAGTTTCCTGAGGCCTCCCAGCT
ATGCTTCCTGTATGGCCTGCAGAACTGAACTGTGAGCCAATTAAACCTCTTTTCTTCATAAATT
ATCCAGTCTTAGGTAGTTCTTTATAGCAGTGTGAGAATGGACTAATGCAGAAGGCATACAACCT
TTAGAATTTGCCCCCTTGAGGGAACAAGATGTGTGAAGCAGGTTCATCCATAGAAAATGTCTGA
GAGAACCTCAAAATCCCTAACCTGACTAACTGATGAAAGTGTTTCTCTCCTAAGGCCAGTCAGT
AAAGACCAGAGGGGGTGACTGTTTCTTTAAATGCAAAGGCAGCAGCACAATAATTCAAGAAACA
TGAAAAATCAAGAAAACATGACACCACCAGAAGAACACAATCATTTTCCAATAACCAACTCCCC
AAAAATGGAGATTTACAAATTGGTTTATAATGAATTCAGAACAATTATGTTAAGGAAGCTCAGC
AAACTAAAAGGAACACCAATAGACTACTCTGTGAAGTCAGGCAAACAATTCATGAACAAAACTA
GAAATTCAAAAAAGAGAAAAATTATCTTAAAAGAAAACCCAGAAGTTATGGAGCTAAAGAATAC
AATGCATGAAATGAAGGAGCGTATCAACAGCAAAGTTGATCAAGCATAAGAAAAAAAAAATCTG
TGAAACTGAAGACTGGCTATTTGAAATTATTCATCAGAGGATTAAAAAAAAAAGAATGAAAAGA
AATAAAGAAAGCCTACAGGATGTATAAAACACCATCAAGAGAACTAATATAAGGATTATTGGAG
TCATAAAGGAGAAGAGAGAAAAGGGTAGAAAACTTATTTAAGAAATAATGGCTGAAAACTCTCC
AAATCTAGGAAAAGATATGAGCATCCAGGTATATGAAGCTCAAAGATCCCCGTACAGGATACAT
TCCAAAAAGACTTCACCAAAACACATGATAATCAAACTGTCAAAAGCAAAATCAAGACGATGAA
TAAACCACCAATCACTAAGAGGGAGACAGGATTCTTATGTTGTCATTATTATTTACATATAATT
TCAGTAAATGTTATTGGAAAATTTATAATGTTTTAAAAAAAGAAATTTGAAAGCACCGAAAGAA
AAGAGACTCATCACATACAGGGAACCCTTTTAAGGCATTCAAGAGATTTCTCAGTAGAAACCTT
ACAAAATAGGAGAGAGTGGGATGAACTATACAAGTGCTGCAAGGAAAAAAATGCCAACCAACGC
TTTACCTGGCAAATCTGTTCCTCAGAAATGAAGGAGAGAGAAGAACTTTCCTAGACAAACAAAA
GCTGAGGCAGTTCATCACCACTAGACCTGCCTTACAAGACATACTAAGGGGAGTTCTTCAAGCT
GAAATGATATGGCAATAGTTAGTAATATGAAATGATAAACCTCACTGGTAAAGGAAAGTACATA
GTCAAATTTAGAACACTTTGATACTATAATGATGGTGTATAAATAATTTTACTGTGCTATGAAG
GTTAAAAGACAAAAGTATTAAAAAAAACCCATAGCTGCAATAGCTTGTCAATGCATACTACAGT
ATAAAAAGATGTAAATTAGAACATTAAAAACATAGCATGCAAGGGTAGGGAAGTAAAAGTGTAG
TTTTCATATGTAATCAAATTTAATTTGTTATCAGCTTAAAATAAATTGTTATACCTATGTTTTA
TGTAAGTGTCATGGTAACTATAAAGGAAAAACCTCTAGTAGATACACAAAAGAAAAAGAGAAAG
GAATCAAAACATAACACTACAGAAAATTATCAAATTACAAAGGAAGACAGCAAGGGAGGAACAA
AGTAAAAAGAGCAAGAAAAAATTTAACATAATGAAAACAGTAAGTCCTTACGTGTCAATAATTA
CTTTAAATGTAAATGGATTAAATTATCCAAACAAAAAAACAGACTGGACAAATGGATTTTAGAA
ACAACAACAACAACAAACACCGCACACACACACACACACACACAAACCACCCAGCCCCAACTAT
GTGCTGCCTACAAGAGATTTACTTCCACTTTAAGGACACATACAGGCTGAAATTAAAAGAACAG
AAAAAGATATTGCATGCAGATAGAAACCAGAAGAGAGGAGAGGCATCTATACTTACAGCATACA
GAAAAGATTTTAAGTTAAAAACTATATCAAAAGGCTAAGAAGGTCAAAATGGTGAAGCAGTTAA
TTGTTCAAGAAGACATAAAAATTGTAAATATTTATACACCCAATATTGAAGCACCTAAATATAT
AAGGCAAATATTAATACATATAAAAGGAGAAATATACAGCAATACAGTAATAGTAGTGAACTTC
AGTGCCTCCCTTTCAAAAATGGATAATCCAGACATAAAATCAATAAGGAAACATTTAACTTAAA
CTTCACTTTAGACCAAATGGATCTAACAGACATTATACTGAACATTTCATCCAACAGTGGTAGA
ATTCACATTCTTCTCAAGCACACATGGAACATTCTCCAGGATAGATTATATGTTAGCTCACAAA
ATAATATTACAAAATTTAATAAAGCTGAAATATCAATTATTTTGCACCACAATAGTATAACACT
AGAAATCAATAACAAGATGGAAACTGGAAATTTACAAATATATAGCATTAACATATTCCTGAAC
AACCAATGGGTCAAAGAAAAAAATCAAAATAATTTTGTGACAGCAAAGTAGAAACACAACATAC
CAAAACATACAGGACACAGCAAAAGCAGTTCTATGAGGTAAGCTTATATTGATAAACACATTTA
AAAAAAGATTTTAAATAAACAACATTACACCTCAAGGAACTACAAGGAAGAAAAAAAAACAAGC
CCCATGTTATCAAAGGGAAGGAACTAACAAAGATCAGACAGAAATAAATGAAACATAGACTAGA
AAAACAATAGAGACTATTAATAAAACTTAGAGTTAGTTTTTTAAAAAATAAAATCAACAAACCT
TTAGCTAGACTAAAAAAAGAGAAGACTCAAATAAAATAAAAAATGAAAGAGGAGACATTACAAC
TGATACCACAGACATACAAATTAAGAGAAAACTATATGCCAACATATTAGTTAACTTGCAATGG
GTAAATCCCTAGAAACATACAACCTACAAAAACTGAATCATGAAGAAATGGAAGATCTGAACAG
ATCAATAATGAATAAGGGAATTGAATCAATATTCAAAAATCTCACAAAAAGAAAAGCTCAGGAT
CAGATGGCTTCACTGGTGAAGACTGCCAACCATTTAAAAAAATTAATACCACTCTTTATTAAGC
TCTTCCAAAAAAATTGAAGAGGAGAAAACACTTTCAAATTCATTATAAGAGGCCAGTTTTACCT
TGATATCAAAGATTTAAAAAGAACACTTTGAGAAAGGAAAATTACAGGCCAAAACCCTTGATAA
ATATAGATGCAAAAATGCTCAGCAAAATACTAGCAAACCTAATTCAGCAACACATTATAATGGC
ATACATCATGACCAAGTGAGATTCATGCCTCGGATGCAGGATAGTTCAATATAATCAAATCAAC
AAATGTTACACTACTTTAACAGAATGAAGGATAAAAATCATATGATCATCTCGATGGTTGAACT
AGTTTACAGTCCCACCAACAGTGTAAAAATGTTCCTATTTCTCCACATCCTCTGCAGCACCTGT
TGTTTCCTAACTTTTTACAGATCACCATTCTAACTGGTGTGAGATGGTATCTTATTGTGGTTTT
GATTTGCATTTCTCTGATGGCCAGTGATGGTGAGCATTTTTCAAGTGTCTGTTGGCTGCATAAA
TGTCTTCTTTTGAGACGTGTCTGTTCATATCCTTCACCTACTTTTTGATGGGGTTGTTTGTTTT
TTTCTTGTAAATTTGTTTGAGTTCTTTGTAGATTCTGGATATTAGCCCTTTGTCAGATGAGTAG
ATTGCAAAAATTTTCTCCCATTCTGTAGGTTGTCTGTTCACTCTGATGGTAGTTTCTTTTGCTG
TGCAGAAGCTCTTTAGTTTAATTAGACCCCATTTGTCAATTTTGTCTTTTGTTGCCATTGCTTT
TGGTGTTTTAGACATGAAGACAGTGTGGTGATTCCTCAAGGATCTAGAACTAGAAATACCATTT
GACCCAGCCATCCTGTTACTGGGTATATACCCAGAGGATTATAAATCACGCTGCTATAAGCCAT
AAAAAATGATGAGTTCATGTCCTTTGTAGGGACATGGATGAAGCTGGAAACCATCATTCTCAGC
AAACTATCACAAGGACAAAAAACCAAACACCGCATGTTCTCACTCATAGGTGGGAATTGAACAA
TGAGAACACATGGACCCAGGAAGGGGAACATCACACACTGGGGATGGTTGTGGGGTGGGGGGAG
GGGAGAGGGATAGCATTAGGAGATATACCTAATGCTAAATGACGAGTTAATGGGTGCAGCACAC
CAACACGGCACATGTGCACATATGTAACAAACCTGCACGTTGTGCACATGTACCCTAAAACTTA
AAGTATAATTAAAAAAAATAATGCTGCTATAAAGACACGTGCACACGTATGTTCATTGCGGCAC
TATTCACAATAGCAAAGACTTGGAACCAACCCAAATGTCCATCAATGATAGACTGGATTAAGAA
AATGTGGCACATATACACCATGGAATACTATGCAGCCATAAAAAAGATGAGTTCATGTCCTTTG
TAGGGACATGGATGAAGCTGGAAACCATCATTCTCAGCAAACTATCACAAGGACAAAAAACCAA
ACACTGCATGTTCTCACTCATAGGTGGGAATTGAACAATGAGAACACTTGGACACAGGAAGGGG
AACATCACACACTGGGGCCTGTCGTGGGGTCAGGGTAGGGGGAGGGATAGCATTAGGAGATATA
CCTAATGTAAATGACGAGTTAATGGGTGCAGCACACCAACACAGCACATGTGTGCACATGTACC
CTAGAACTTAAAGTATAATAAAAAATAAATCATATCATCATCTCAGTAGATTTAGAAAAGCATT
TAACAATATTCAACATCCTTTCAGAACTAAAAACTCTCAATAAATCAGGTATAGAAAGAATGTG
CCTCAACACTATAAAAGCCACATATGACAAACCTGGAGGTAATATACTCAATGGTGAAAAGTAA
AAAGCTTTGACTCTAAGATCAGAACCAAAACAAGGATGTCCATTCTCACCACTTATATTTAACA
TAGTAGTTGAAATTCTAGCTAGAGCAATTAGGCAAGAAAAAAGGCACCCAAGTTGGAAAGAATG
AAGTTAAATTGTCTCTGTAGATGACATGATCTTATATATAGAAAACACTAAAGACTCCACCAAA
ATGCTGTTTTAATTAGAGCTTAAAAAAATAATTCACTAAAGTTGCAGGATACAAAATCAGTATA
CAAAAATCAGTTGCATTTCTAAACACCAAAAACAAGTTATCCAAAAAATTAAGAAAACAATCCT
ATTTGTGATATCATCAAAAAATAAAATACTAAAGAATACCAAAGAAACTGAAAATAAATGGAAA
TAAATGGAAAGATAGCCCATGTTCATGGATTAGATGAATTAATACTGTTAAAATGTTCATACTA
CCCAAAGCAACCTACAGATTAAGTGCAATTCCTACTAAAATTCCAATGACATTTTTCACAGAAA
TAGAAAACACACTCCTAAAGTTTGCATGGAACCGCAAAAGACTCAAATAGATGAAGCAATTTTG
AGCAAGAACAGTAAAGCTGGAGACATCACACTACCTAACTTCAAAATTTTATTAATCAAAACAG
CATGACATAAAAACAGACAGAAAAGACCAATGGAACAGAATAGAGAGCCCAGAAATAAACTCAC
GTTTATAGAGTCAACTAATATTCAACGAAGGTGCCAAGAGTACACAATGGGGAAAGTATAGTCT
CTACAATAAACAGCACTGGGAAGACAATATCCAAATGCAAAAGAATGAAATTACACCCTTATCT
TATACCATACACACAAATCAAATCAAAATTGATAAAGACTTAAATATAAGACCTGAAACCATAC
AATCTTTAGGCAAAAACTCATTGACATTGGTCTTGGCAATGATTTTTTTGATATGACACCAGAA
GCACAGGCAACAAAAGCAAACCTAAACAAGTGGGACCCTAACAAACTAAAAAGTTTCTGCACAG
CAAAGGAAACAATCAATCATCAGAATTAAAAGGCAATTTATGAAATGGGAGAAAATGTTTGCAA
ACCACATATCTGATAAAGGGTTAATATCCAAAAATATATAAGGAATGCATAAAATTCAATAGAA
ATAAACAAACAAATAATCCAATTTTAAAATGGGTGAAGAACCTGAATAGACATTTTTTCAAAGA
AGACTTACAGATAGGCAACAGGCATATGAAAAAATGCTTGACATCACTAATAATCAGGGAAATG
CAAATCAAAGCTCCAGTGAAATACCACCTCCAACTATTAGGATGGCTATTATCAAAAACTCAAA
AGAAAACAAGCTGGGGGAATGTAGAGAAAAGGGAAAAGAAGATCCTTATACACTGTTGGTGTGA
ATTTAAACTGGAATAGCCCTTATGGAAAACAGCATGGAGGTTCCTCAAAAAATTAAAAATAGAA
CTACTATATGATCCAGCAATTTCACTATTGAGTATATATCCAAATGAATTAAAATCACTGTCTT
GAAGAGGTATTTGCACACTCATATTTATTTCAGCATTATTCACAATAGCCAAGACATGGAATCA
ACCTAAGTGTTCATCAGTAGATGATTAGATAAAGAGAACGTGGTATATAGACACAGTGGAATCT
ATTTAGTGTTCAAAAAGAAGGAAATCCAACTTTTAAAATCCTTTAAAAAGTTAAACTCATAAAA
ACAGAGAGGAGAATGGCGGTTTCCAGGAACTGGAGGGTGGGAGAATGGGGAGATGTTGGTCAAA
AGGTACAAACTTTCAGTTATAAAATGAATAAGTTCTAGAGATCTAGTGTACAACAGCATTACTA
TAGTTAATAATAATATTTTTTATACTTGAAATTTGCTAAGAGTAAATATCAATATTCTCAATAC
ACACAAAACAGAACTATCTGAAGGCACTGATATGTTAATTATCTTCATTATAGTAATCATTTCA
CAATGTATAATGAATATCAAAACAATAGTGTACATCTTAAATATATACAGTTTTGATTTGTTAA
TCATACATCAATGAAGCTAGAAAAAATGTTGTAATTTTTAAAACAATAGTAATATAAATTAGGG
GTGAAGGAATTGACCTATGTTGGAGAAAAGTTTTTGTAAACTATTTAAATTAATTGGTATCCAT
TCAAGCTAGATTATTTTTAATTGTTAATTTAATTGTAATACTAAGGCAACCACTAAAAAAGCCT
TTAAAAAAATATAGCACTTGAGGCTGGGCATGGTGGCCCTCAATTATATATATAATATATATAA
AATATATATTATATATATAGTAGATGAACAACAATGGGATTTAAATGGTACACTAGAAAATATC
TGTTTAACAAAAAGAAAGCAATAGTGGAGAAATATAAGAACAAAACCATGTAAGATTTATAGAA
AATGAATAGCAAATTGGTTGACCTAAACCCTATCTTATAATTATATTAAAGATAAATGAAATAA
ATACTACATCAAAAGGCAGAGATTATCAGAATAGAGAAGAAAAATCCAAACCATAATTCAACCT
TATGTTATCTGTATTTAGAATATTTAGAGTCAAGACACAAATAGATAGAGTTCCATTTGGGCGT
GAAAATATTTACCATGCAAAATGTAATTAAAATAGAGCTAGAGCAGCAATACTAATATCTGACA
AAATATACTTTAACAAAAATTGTTGCTAAAGACAAAAAAGAACATTTTATAATAAGACACAACA
ATTATAAACATATACACCAAACAATAGAGCCCAAAATATACAAAGCAAAAACTGCTAGAATTGA
AGAAAGATAGAAAAATCAATGATTACAGTTGGAGGTGTCAATACCAAACTTTCAGTAATACACA
GAACAACAAGTCAAAAGCAAAAAGGAAATAAAAAACTACACATTATCATACAACACCTTAATAC
GATATCCAACAATAGCAGAATACACATTATTCTGAAGTGCACATGGAATATTCTTCAGGATGAC
ACATATTAGGCTGTAAAACATATCTTAATAATTTAAAAGAATTGAAATAATACAGCACATATTC
TCTGACTGCAAAATATTAAACCAATTACAGAAGAAAATGTGGGAAATTCACAATGATATGGAGA
TTTAAAAATACTTCAAAGTAACCAATGAATCAAAGAACAAATCACAAGAGAAATTAGAAAATAT
TTTAGATGAATAAAACTGAAGAAACAACATACCAAAATTTTGTGGTTGTAGCTAAAGCAGTGCT
TCAAGGGAAATGTATAACAGTATATCCCTAAATTTTAAAAAAATCTCAATCCAATAACCTAATT
TTTCACCTTAAGAAATGAGAGTGAAAGAGCAGACTTAACCCAAAGTAAACAGAAGGAAGAAATA
ATAAAGATTAGCATGGAGATAAGTAGTGAAAATAAAAACAATAGAGAAAATTAATAAACTTGAA
AGTTGGTTCTTCTGATATATCAGTAAAATTGACGTATTATTAGTTTGACCGAGAAAGAACAAAA
GAGAGAAGATTCAAGTTACTAGACATAAATGAAAGTATAGATATCACCTTACAGAAATTAAAAG
AATTCTAACAGAATATTGTGAAAAAGTGAATGTCAAAAAATTAAATTATATGAGATACACAAAT
CCCTCTAAAGGCACAAACTACCACAGCTGGTGTAAAAACATGAATACACCATTTACAGTTAAAA
AGACTGAATAGATAAAAATTTTAAGAAGAAATTGAGTAAGTAATTTAATTTTCAAACTATAATC
CCAAGACCAGATGTTTTCATTGGTGAATTCTACCAAACTTTAAAAGGATTAATATCTATTTTTC
ATACACTCTTTCCAGAAAATAGAAAAGGAGGGAACACTCTATAACTCACTGTATGAGGTCCGTA
TTACCCTTATATCAAGACCAAACATCATAAGAAAAGAAGACTAAAGACTTATGGTTTCTGCTCT
GATATGTTTGGAAGTCATCACTACTATTGTCACAAGGAAAAATCTGAACAAACTAAAGTCAACG
ATTTCTTAAACTAACCATAGAATTGAGGTAACGGGCGAAAACTGGAGATGTAGGCAAATACAAA
AAAAATCACAGTTTATCAGGAGCAGAAACTGCTGAAACCAGCAACTCGTATGAACATGTTAAGT
GGTAATTGACAAATTTCTGGAGATTGAATGTGGACTGGATTGAGAGTTAGGAACTCCTAAGTGC
CCAGTTTTTGATGACCCCACACACTTTTGTAAACTAGACTTCCAAGAGCCCCAGCAAGTTTCTT
ACAGTGAAGACTGCAGAAAAATCCCCTGATGCTTCAGATAGGAGGAAGGGAAAAGCAACTATTT
TGAAATAAGCCCAGGGGACAAGTAGTTATTTCTAAACACTCTCAGAGCATTTTCTTTCACACGG
CAGGGGGCTCCCTGCAAGGGAAGCTACTTTGCCTGAGCCTTGTCTGATGTAGGAGAAAAGGAAT
TGGATGGCTCAAGCTCCATCTAGCCTTTCTGATTTATATAAGGGAAGCAAAAAATAGGTTAAGA
AACTCTTCTGAAAGTCACAACTCAGATTTATTTTATATTTATTTATTTATTTATATTTTTTGAG
ACGGAGTCTCGCTCTGTTGCCCAGGCTGGAGTGCAGTGGCGCGATCTCGGCTCACTGCAAGCTC
CACCTCCCGGGTTCACGCCATTCTTCTGCCTCAGCCTCCTGAGTAGCTGGGACTACAGGCGCCC
AGCTAATTTTTTGTATTTTTAGTAGAGACGGGGTTTCACCACGTTAGCCAGGATGGTCTCGATC
TCCTGACCTCGTGATCCACCTGCCTCTGCCTCCCAAAGTGCTGGGATTACAGGCGTGAGCCACC
GCACCTGGCCTCACAACTCAGATTTAACCATAAGATTATAGAACACTTCTCCTCCCAAAACAGA
GATATAGATCAATGGAACAGAACAGAGCCCTCAGAAATAACGCCGCATATCTACAACTATCTGA
TCTTTGACAAACCCGAGAAAAACAAGCAATGGGGAAAGGATTCCCTATTTAATAAATGGTGCTG
GGAAAACTGGCTAGCCATATGTAAAAAGCTGAAACTGGATCCCTTCCTTACACCTTACACAAAA
ATTAATTCAAGATGGATTAAAGACTTAAACGTTAGGCCTAAAACCATAAAAACCCTAGAAGAAA
ACCTAGGCATTACCATTCAGGACATAGGCATGGGCAAGGACTTCATGTCTAAAACACCAAAAGC
AATGGCAACAAAAGACAAAATTGACAAACGGGATCTCATTAAACTAAAGAGCTTCTGCACAGCA
AAAGAAACTACCATCAGAGTGAACAGGCAACCTACAAAATTTTCACAACCTACTCATCTGACAA
AGGGCTAATATCCAGAATCTACAATGAACTCAGACAAATTTACAAGAAAAAAACAAACAACCTC
ATCAAAAAGTGGGCAAAGGATATAAGCAGACACTTCTCAAAAGAAGACATTTATGCAGCCAACA
GACACATGAAAAAATGCTCATCATCACTGGCCGTCAGAGAAATGCAAATCAAAACCACAATGAG
ATACCATCTCACACCAGTTAGAATGGCGATCATTAAAAAGTCAGGAAACAACAGGTGCTGGAGA
GGATGTGGAGAAATAGGAACACTTTTACACTGTTGGTGGGACTGTAAACTAGTTCAACCATTGT
GGAAGTCAGTGTGGCGATTCCTCAGGGATCTAGAACTAGAAATACCATTTGACCCAGCCATCCC
ATTGCTATATATATATATATATACCCAAAGGACTATAAATCATGCTGCTATAAAGACATATGCA
CAGGTATGTTTATTGCAGCACTATTCACAATAGCAAAGACTTGGAACCAACCCAAATGTCCAAC
AATGATAGACTGGATTAAGAAAATGTGGCACATATACACCATGGAATACTATGCAGCCATAAAA
AAGATGAGTTCATGTCTTTTGTAGGGACATGGATGAAATTGGAAATCATCATTCTCAGTAAACT
ATCGCAAGGACAAAAAACCAAACACTGCATGTTCTCACTCATAGATGGGAATTGAACAATGAGA
ACACATGGACACAGGAAGGGGAACATCACACTCTGGGGACTGTTGTGGGGTCGGGGGAGGGGGG
AGGGATAGCATTAGGAGATATACCTAATGGTAAATGACGAGTTAATGGGTGTAGCACACCAGCA
TGGCACATGTATACATATGTAACAAACCTGCACATTGTGCACATGTACCCTAAAACTTAAAGTA
TAATAATAATAAAAAAAGGCTACCTAAAAAAAAAAAAAAGAACACTTCTCCTCCCAACACCATA
TCACCACATCAACCAGGACTCCAGTGTAATAGCAGTGAATTCTAACTGAAAGAGGTGAAAGACA
CTGATTGTATTTAAGAAAGATCTTCTAAGGAAATCCAAAAATAGTAGGGGAGATCAAAACAAAG
ATACTAGAGGAAATTGAATATGTGACACCTATAGCTACAAAAAAATTAAACATAACATAGCCCT
AACCATATAAACATAAAACCTCACACAAAGACCTATTATCTGAGATTCTGTTGCCTGATACATT
GCGTTTTATTTCAATAAAAAAATTAGAGGGTATGTTAAAAAGCAGGAAAAGTTAGTCTAAAGAG
ACAAATTGAGCCTCAGAAGTAGGCTCAGATATGGCAGAGATTTGGCAATTATACCTAGAGTTTA
ATATAAATGATTAATATAATAAGTGTTCTAACAGAAAAAGGCAACATGCAAGAACGGATGGGTA
ATGTGATCAGCAAGAAGGAAACTCTAAGAAAGAAGTCAAAAGGAAATGCTAGGAATAAAAACCT
ACAAGAAATAAAGAATGCCTGTGATGGGTTCCTCAGTAGACTGGACAAGGTCAAAGAATCAGTG
GATTTGAAAATATGTCAACAAAAACTGCCCCACTGAAATACAAAAGAAAAATAGAATTTTAAAA
ACGTAACACAATCTCCAAAACAGTGGGACAATTACAAAAGATGTAATGTGCCTAATGCAAATGA
CAGTAGGAGTATAAAGGGAGAAAGGAATAGAAAATCTGAAGTAATAATGGCAGAGAGTTTTCCA
AAATTAATGCTAAACCACAGATACAGCAAGCCCAGAGAACAACAAGGAGGAAATTTAGTAAAGC
GTCTGCAACCAAGTATGTCATATTCAGACTGACAAAACCAAAGGTGAAGAGAAAATATTGAAAG
AAGACAAAGAGGAAAATAAATATCAAGAAAATACATACGAAATACATCATACATACATAAGAAA
TACATCAGACCATACAAGCAAGAAGAGAATGGAGTGAAATGTTTAAAATGTTGAAAAAAAAACT
ATCAATTTGCAATTCTGTATCCAGTGAGATTATCTTTCAAAAGTGAAGAGGGAAATGGCAGAGA
AGTCATCTCCAAGACCTATGGTTTCCCTTCACAGAAACACTGAAAAATATGAACAAAAGTGGTC
AGAATTAACTTTCTAAGAATTCTATAAAATGGTAAAATGTTTACACCAGTAAAGCAAATGCTGA
ATTGAGAAGGCAACTTAAAAAGGTGAAGAAAACTTCGTATTATTTTTATGTGTCCTTGCCCCAC
GTCCTTCCCTACCTTAGTCTTGAAGATGGCAGCCCACATTTCTACTGTGGGGCTCTGGTTTCTG
TTTCCTGGTTCAAGAGGGAGAATAACAGACCTTACTTTTAGTCATTATTATTTCCTTCTTTCTG
ATTTCCTTGGGTTTATTTTGCTCTTCTTTCTACTTTATTGAAATGAGAACTAAGATTATGATTT
GAGACATTTTTCTAATGTAAGCATTTAGTGCTATAAATTTCCATCTCAACACTGCTTTAGTCAC
ATCCCACAAATTTTTATATGTTGTAATTTCACTTTCATTTAGTTCTATTTTTAAATTTTTTCTT
TTTATACTTCCTCTGACTCACAGATTACTTAGAATTGTGTTGTTCAGTTTTCAAGGATATTGTA
GATTTTCCTGTTTCTCTGTTGTCTAATAGTTCTGTTCCATTTTGTACAGATAGCTCACGCTGTA
TGATTTCAATTTTTTAAAAAATTGTGCTTTGTTTTATGGCCCAGATATGGTCTGTGCTGTGAAT
ATTCCATGTTATTATAAAGTATGCCTGTTATATTATTATATATATATAATATATATAATTATAA
AGCATGCCCGTTTTGTATTGTTAGCAGAGTATTCTAGAAATGTCAATGAGATCTTGTTGGTTGA
TGGTGCTTTTCAGTTCTATATCTTTGCTAATTTTTTTTTTTTTTGCTTAGTAGCTATATGAGAT
TCTGAGAGAGGAAATTGAAGTCTCCAACCATAATTGTGGATTTGTCTATTTCTCCTATCAGTTC
TATCAGTTTGTGCATCACATATTTGAGGCTCTGTTGTTTGGTGCATACACAAGTGGAATCATTG
TGCCCTCTTGGTGGCTTATTTTATGATTATATAGTGCCTATCTTTGTGGTATTTTTATTTGCTC
TTAAATCTACTTTGTGTTATATTCATATACCCATTCTTTTTTAAAAAAAATTGTTTGCGTGATA
CATCTTTTCCATTCTTTTAATCTCAGCCTATCTGTGCCATTGAATTTGAAGTGAGTTTTCATAT
AGAGAACATATTATTGAATCATCATTTTAAAAATTCCTTTTGCCAATCTTTTTTATACTGAGGT
AAAATTGACATAAAATTTATCATTTTAAAGTGTACAATACAGTGGCATTTGGTAATACACATGT
TATGCAACGTTAACTCTACCTGGCTCCTAAATGTTTTCATCATCCCCAAAAGGAAACTTCATAC
TCATTAAGCAGTTAATTCCCATTCCTTCTCTCGGCCACTGGCATCCGCAAACCTACTTTTCTGT
CTCTATGAATTTACCTATTATGGATATTTTGTATAAATTGAATTATACAATAAGTGACCTTTAT
GTTTGGCTTCGTTCGCTTCGCATACTATTTTTCGATATTCAACCATGTTGTAGTATGTATCAGT
TTTATTTGAATAACTCAATTCTTTTTGTTGTATAGCTAAAAGTTGATTCCTAGGTCATAATGAT
AATTCTATGTTTAGTTTATTGAATAGCTGCCAAAGTTTTTCCACAGTGGCTCTGTCATTTTAAA
ATCCCACTAGCAATGGATGAGAGTTCCAATATCTCCACATCCTTACCAATATTGTTATTTTATA
TTTTTATAATTATAATTTTCCTAGTGAATACGCAATGGTATCTCATTGTGTTTTTGGTTTGCCT
TTCCCTAATGACTAATGATGTTGAGCATCTTACAATGTACTTGTTAACTATTTGTGTTCTTTAG
AGAAATGTCTATTCAAGTGCCTTGTCCATTTTAAAAATCGAGTTGTCTTGTTGACTTATGAGTT
CTTTAATACAGTAAACGCTTATCAGATATGATTTATAAGTATTTTAACCCATTCTGAAGGTCAC
CTTTTCACTTTTGTGGTAGACCATTATGCACAAAGGTTTTAATTTTGATAAATCCAATTTATCC
GCTTTTGTTGTTGTTTTTGTTGTTCGTGCTTTTGCAAAACCTAGTGTCATGAGGTTTTCTCATT
ATCTTTGGAGAATTTTATAGTTTGGGTCTATACATGTAGATTATTGATCTAATTTCCATTAATT
TGTGTGTATGCTACGAGGTAGGGGTCCAAATTCAATTTTTGCATTGAATTGAAAATTCATATTT
TCAGTTTCAAATTTCAACTGCATATTCAGTTGTTGCAGCACAATTTGTTGAAGAGATCATTCTT
TACCACAGGGAATGATCTGGGACCCTTGTCAAAAATCAATTGATCATAGATGTATGGGATTATT
TCAGACTTTAGATCTTGTTCCATGAATATGCCTATTTTTATGCCAGCACTGCAGTATTTTCATT
ACTGTAGCTTTATCATAAATTTTGAAATCAGGAAGTATGTATCCTCCAAGTGTGATTTTCATTT
GCTAGAGTGTTTTGACTATTTGGGGTCTTTGCAATTCCATATGAATTTCAGAATTGGCTTTTCA
TTTAAAAAAATGGTAGTTGAGATTTTCATAGGAATTATATTGAATCTACAGATCACTTTGGGTA
GTATTGCCATCTTAACAATATTGTATTCCAATCCATAAACACGGATGTATTGCCATTCATATCT
TTTTTTCTTTTCTTTCGGCAACATTTAGTATATGACACTTGTAACTCCTTGGTTAAATTTATAC
CTAAGCATTTTATCCTTTCTGATGCTGTTTAAATGGAATTATTTTATTAATTTCTCTTTTAGAT
GGCTTGTTGCAGGTGTATAGAAATAGAACTGATTATTGTGCTTTTATTGAATTATCTGAAACTT
TGCTGCATTTATTAACTCTAGTAGGTTTTTTTTTTCTTTAAAGTTGTCTATATATCTTGCTCTG
TGAATAGATAATTTTACTTCTTGATCTCCAATATGGATGCCTTTCTTTCTTTTTCTTACCTAAT
TGCTGTAGCTAGAATTTTCAGTATAATGTATGATAGAAGTTGTTCACAACTATCCTTGTCTTCT
TTCTGATCCTAGGGGTATAGCTTTCAGTCTTTCACCATTAAGCACAATGTTAGCTGTGGGGTTT
TCTTAGATGCCATTTAATATATTAAGGAAGTGCTCTGCTATTTCTAATTGGTTGAGTATTTTTA
TTATAAAATGGTGTTAGATTTTATGACTTTATGTACTGCATAATTGAGATTATCATGTGGTCTT
TTCATTCGATTAATGTGGTATATTTTATGATTTTCATATGTTGATCCACCTTTATATTCTTGGG
GCAAATCCCACTGTGTACACGGGTTTTTTAGGGTCTCCTAATTTTCTTCATTCTTTTTCTTTTC
TCTCCCTGAGACTGAATAACGTTAACTGACCTATCTTCAAGTTTACTATTTTTTCTTCTGCTGT
TCAAATCTGCTTGAACCTATAGAGTGAATTTTTCATTTTACTTATTGAACTTTTCAGCTCCAAA
ATTTCTCTTTGGCTACTTTGTATAATATCTATCTCTCTATAGATATTCTCTATTTGGAAAGACA
TTTTTCTCCTGGTTTTCTTTACTTATTTGTATTTTTTAAAGTCTTTAAGCATATTTGAGACAGT
TGATTTAAGTATTTGTGTACTAAGTCCATTGCCTAAGCTTTTGCATAGAGATTTTATATTAATT
TCTTTTTTTTCTGTGAACAGGTCTTTTTTTTTTTTTTTTTTTTTTTTTTTTTTTTAGAAAACTG
GACATTTTGAGTAGTATAAACGTGGCTATTTTAGAAATAATTTTCCTCCCTCCTTAGGTTTTGT
TTCCACTTGTTGCATGTTGCTGTTGTTTGCTCATTAGTGACTTTTCTAAAGTATTTTGAAAAGT
CTGTGTTCTTGATCATGTGGGTCCATTGAATTCTGTATTCTGTTAATTTCATAGTCAGCTAGTG
TTCTGAAAGTTCCCTTAAGTGCATAGAGCCAATAAAAGAAAAAGAAAAGAAACACAGAAAAAGA
AAAGAGGAAAAAAGGAGGGAAAGAGATTTAAAAAAATAATGTCGGTTGGGCATGGTGGCTCATG
CCTGTAATCCCAGCACTTTTGGGAGGCCGAGGCAGGCAAATCACTTAAGGTTGACCATCCTGGC
CAACACGGAGAAACCTTGTCTCTACTGAAAATATAAAATTAGCCGGGCATGGTGGCACATGCCT
GTAATCCCAGCTACTCGGGAGGCTGAGAGGTAGGAGAATCACTTGAACTCGGGAGGTGGAGGTT
GCAGTGAACTGAGATCACACCACTGCACTCCAGCCTGGGCAACAAGAATGAAACTCCATCTCAA
AAAAAAATAATAATAATAATGCCTTTGCCGATTTGCTCTGTGTTTGGGCCCTCATTCAATGCCT
AGTCAGGCCATTTACAACTCTCTCTTAACTTTCACTACCTGCTTGTGTACTGACTGAAGGCCAC
CTATAGGTGAAAGCTTAACATCATCTCAGATCTTTTTGGACCATGAATCCTACCCTGGGTATGC
ACATGATCTTCTTAATTTCCCAGTAGATGCAAGAGTTTTAGTGTTTTAAAAGTCCTTATTCCCT
CATCTATCTTCTTTTCTGACCTTTTTCAGTCTGCTTATTGTTCATCTGAACTGACATCCTTTGC
CCCAAGCGGCTGTGGCAAAAACTTTTACCTTTAAATGCTTTCACCACAAGCCACTTGGGAAGCT
GCCCCAGACCTGGGACTGCTCTGACCCTGATGAAACAAAGACAAGACCTTGTACAGCCAGGCAG
CCATAAGACAAGTCCATACCCAAACCACAGTTCTTTCAGAATAAGGTCTATATTGGATTCTCTG
GCCCTAGTAACCAGCATGAGTCTGGGCTTGCCATCTTCATGGCCACTTGTCTTAGTTTGCTAGG
GCTGCCATAACAAAATATGAGAGACTGAGTGGCTTAAACAAGAGAAATTTATTTTTTCACAATT
CTGAAGACCAGAAGTCCATTACTCTGAAATCTCTCTCCTTGGCTTGCAGATACTGCCTTCTTGC
CGTGTCCTCACACAGCCTTTTCTCTGTGTACACATCCCTGGTGTTTTTTAAGCCTGTCCAAATT
TTCTGTTCTTCTGAGGACACCAATCAGATTGGATTAGCGACCACCCATATGATCATTTTACCTT
AAGTACCTCTTCAAAGGTATCAGAGTAGTATATTTGCTACAGTTGATGAACCTGCATACAACAT
CATCACTTGAGGTCTGCAGTTTACATTAGAGTTCATCGTTAGTGTTATATATTCTAGGGTTTGT
TTTGTTTTGAGACAGAACAGAGTCTTGCTGTGTTACCCAGGCTGGAGCGCAGTGGTACAATCTC
ATTGCTACCTCTGCTTCCCAGATTCAAGCAATTCTCATACCTCAGCCTCCTAAGTAGCTGGAAC
TACAGGCGCACACCACCATGCCCAGCTAATTTTCGTATTTTTAGTGGAGATGGGGTTTCACCAT
GTTGGCCAGGCAGGTCTTGAACTCCTTGCCTCAAGTGATCTGCCCGCCTCAGCCTCCCCCAGTG
TTGGGATAACAGGCATGAGTCACCATGCCTGGCCTTATTCTATGGGTTTTGAAATGTATAATGA
CATGTATCCATCGTTGTAGTATTAGATAGAATAGCTTCATTACCCTAAAAGTCTTCTTTGCACT
GGGTTGAGTTTTAAAAGCTCTTTGATTATTTTATGACAGTTCCTTATTAGATATATCTTTTGCA
AGTATTTTTATCAGTCTGTGGTTATCTTGTCTTTTACAGAGCAGTAATTTTTAATTTTAATAAA
ATCCAATTTGTCAATTACTTATCTCATATGACTCTGGTGTTACATCTAAAATGTTACCACCATA
CTCAAGGTCACCTAGGTTTTCTCTAGGAATTTTATGGTTTTGCATTTTACATTTGGTGTATGAC
CCATTTGAAGTTAATTTTTGTGAGGTTGTAAGGTCTGTGCCTAGATTCATTTTTTTTTTTTTTG
GCATGTTACTTCAGTATTCATAAGAAACATTGTTCTGTAATCTTCTTTCTTATAGTATCTTAGT
CTTGCTTTAGTTTTTGGGCAATGCTGGCCTCACTGAATAAATTCAAAGTGTTCCCTCCTCTTCA
ATTATTTGGAAAAGTTTGAGAAAGACTATTGTTAACTGTTTTCTAAATTTTTGGTGGAATTTAC
CAGTGAACCAACTGGTCCTAGGCTTTTCTCCAGTAGGTGGTTTTGATTATGCTTTCAATCTCTT
TACAAGTTACACATCTATACAGACTTTTATAATTCAGTCTTGGTAGGTTGTGCGTATTTAGGAA
TCTGACCACTTCATCTAAGTTATCCAATTAGTTGGCATGCAATTATTCGTAGTTCTCTGAATAA
TCATTTTTATTTCCACAAAATTGGTAATATCCCAGTTTCCATTTTTTATTTCATTGAATCTTCT
TTTTTCTTAGCTAATCTAGCTAAATGTTTGCCAATTTTGTTGATCTTTTGGAAGAACCAACTTT
TGATTTATTAATTTTCTCTACTCTTTTTCTGTTCTTTATATTATTTATTTCCACACTAATCTTT
ACTATTTTCTTCCTTCTGTTGGCCTTTAATTTTTTTTTTTTAATTTTTAAGGTGTAAATTTAGG
TTGAGAAATTTTTTAAATGAAAGCATTTAGAGCTATAAATTTTCCTTCTGGTGTTCCTTTCACT
ACCTGCCATAAATTTTGATATGTTGAGTTTTTGTTTGTCTTGGAGTATTTTCTAATTTGTCTTC
TAATTTCTTCCTTGACCTATTGGTTATTTAAATGTATTTAATTTTTGCATATTGTGGATTTCCC
AGTTTTCCTTCTGTTATTGATTTCTAGTTTTATTCCATTGTGATCACAGAAGATATTTTGTATA
ATCGCAGTCTATTAACATTTATTAAGTCTTGTGGCCTAACAGAGGATCTATGTTGGAGAATGTT
CCAAGTGCAATTGAGAATACTATTCTGGTGCTATTAGGTGAAGTATTCTCTATATGTCTGTTAA
GTCCAATTCATCTATAGTGTTGACGTTTCCTGTTCCTTACTGATTTTCTGACTTATTATTCTAT
CCATTATTAAAAGTGGAGTGGTAAAGTCTCTATTATTGTAGAACTCTCTGTTTTTCAAGTCTAT
CAATATCTGTTTCATATATTTTGGAGCTCTGTTTGCTGCATATGTGTTTACAATTGTTATATCT
TCTTGGCAAATTGACCAGTTTCATCAACATAAAATATAATTCTTATTGTCTTCTAACAGTTTAT
TTTTCTTTTTACATAAAGCCTATTTTATCTGATCTTAGATTCCCTCACACTCCACCCCAGCACG
CTTTTGGTTACATTTACATAATATATCTTTTCCATCCTTTCATTTTCAACCTGTTTGTGTCTTT
AGATCTAAAGTGAATGTCTTACAGACAGCATAAGCTATGTCATTAAAAAAATCCATTCTGCTTA
TCTCTGCCTTTTGACTGGGGAGTTTAATCCATTTGCATTTAAAGTAATCACTGATCATTAAATA
CTTTCAGTATTTTGTTGTTTTATGTATGTCTTATAACTCTTTTGCTCTTCATTTCCTTCAATAT
TGCCTTTGTGTTTAGCTTATCTTTTTGTGTCACACTTTGATCCCCTTCTCATTTCTTTTTTATA
TTTTCTTTGTGGTTACCAGGAGGACAATGTATCAACTTTTAAAGTTATTACAATTTTATTTTTT
TAAATCTCTCCCATTCGGGGATTTTAGGAAGGTTAAATAATAATGTAAATGAGATACCTAGAAC
AATATAAGCATTCAGGAATTATTAACTCAATTCCAATCCTTCCTCCACCTCCACCTCTTTCTCT
GTGAGATTATAGAAAAGATGACAAAAAGGATGTTTTCTGAGCCCTTTAATTGTTGAGAATGATC
TTTGAGAAAAAGAAAAAAAATGAAAGCACTAGGAATGTACAACAGCCTGGAAGTATAATTAAGT
GTAAATTAAATAGATAAAAGTTATAAGCAGAGGAAAGTATAGTAGAACTCAGTATTTAAAAGAG
AATCAATGTGAAAATTATATAAATTTATGTAAAATAAAACTACCAGACAAATCTGATATCCTTA
GGATTTTTCTTTCTTTCATGTGATTTCTAATTGCTACATATGACACTAAACCATTGATCTGAGC
TGTAAGAGAAACTGGAAATTGTTCTGTTATCTTTTGTAAGATTTCTAGAACATTTTGCCCTCAG
ACTTAAATGCCAACGTATTTCTCACTTATTGTTTACTGCTTTTGGATTTACATATGATTTGATT
CTTTCTTATCTCTTATCCTTACAATGTAATTCAAACTGATGCCAATTTAAGTTCAATTGCGTAC
AAAAACTCTACTCCTATGCAGCTCCGCCCCATCTAATTTACATTATTGATGTCGCAAATTCCAT
CTTTGTACATAGTTTACATATTAACATGGATTTATACATTTTTATGTATTTGGTTTTTAAATCC
TGTAGAAAATAAAAAGTCAACACACCAATATTAAAATAATACTGGTTTTTATATTTGTCCATGT
GCTTACCTTTATCAGTGTTCTTTACATCTTTATACGGGTTTGAGTTACTGTCTTGTGTCCTTTA
GTTCCAACCTAAAGAACTCCCTTTAGCATTTTTATAGGGCAGGTCTAGTGGTAATGAACTCTCT
GAGTTTTTATTTAGGGATGTCTTAATTTCTAGCTCCTGTTTGAAGTAAATTTTTCTGGATATAC
AATTCTCTGTTGATTGATTTTTGTTCATTTTCCCTTCAGCTCTTTTAAATACATTATCCCACTT
TCTTCTGCCTTCCAAGGTTTCTATTAACAAAATTCAGCTTATAATCTTATTAAAGATCTCATGT
ACATGAGTGGCTTCTCTCTTGCTGTTTTCAAGATTCTGTGACTTTGGTTTCTGATAGTTTAAAT
ATAATGTGTCTTATTGTGGGTCTCTTTGGATTTATCCTAGAGTTTCTTGGTCTTCTTACGTTGG
TATATCCATGTATTTCAAGACATTTGAGTAGTTTTCAGCCATTATTTCTTCAAACAATCTCTCC
TCTTTGGGGACTTCCATTTTACCTATATTGGTTCTTTTGATGGTGTGGCACCAGTCCCCTAGAC
TTTGTTCACTTTTTTCCAGTCTTTTATTTCTGCTTCTCAGACTCAACAGCTTCAAGTGTTCTGT
ATTCAAGTCTGCTGACTCTTTCTTCTTCCAGCTCAAATCTGCTGTTGGATCCCCCCTTGTAAAA
TTTTTAATTCCATTTTAGTGTTTTTCAAGTTAAGTATTTTTATTAGGTTCCTTTTTATAATTTC
TTTTTGTTGATATTCTCATTTTATTACACATAATTTGTCTGATTTCCATTAGTTTTTTTCTTTG
TTTTCCTTTAGCTCTTTGAAAATATTTAAGACATTTTAAAAGTCTTTATCCAAGTTCAATTTCT
ATGGTTCTGTAGAGATATTTTCTGCCAGTTTATGTTCTTCTTTTCCATGGGCCATGTTTTCCTG
TTTCTTTGTATACTTTCTAATTTTTGGTTGAAAACTGAGCATTTGAAAATAGAGCCAACTTTCC
CAGTTTCTGCAGAGAGTCTTTATGCCACAGTATTCGTTCACTGATTAACTGGGTATATCTAAGC
TTAGGGAGCAGCTGAGTCAAAAGTTTAAGGTCTTCTCAGGTCTTTTCTGAGTATACATGTTTCC
TATGCCTGTGTGAAATGTTCTCAATTTCCCTATATAAACAGCTACTTCTTCTTTTTTTTTTTTT
TTTGAGATGGAGTCTCGCTCTGTCACCCAGGCTGGAGTGCAGTCATCTCAGCTCACTGCATCCT
CCACCTCCCAGGTTCAAACAATTCTCCTATACAGGTGTGTGCCACCACGTCTGGCCAATTTTTG
TATTTTTAGTAGAGACAGGGTTTCGCCGTGTTGGCCAGGCTGGTCTCAATCTCCTGACCTCAGG
AGGATTACAGGCTTGAGCCACAGTGTCCAGCCTAAACAGCTACTTTTGAATGCTTTAATTTCCT
GAATAGTCTCAACCCAGTTTTTCCTTGAGGTCTTAGGTGGTCCATTGTATGTCTCCACCCATAG
TTGCTTGCCCCAGGCATCTGTGGGTCTGTGGTACCACTGCAGCTTTCACCACCTGTAGCTGCCA
CCTTTCCCTATCTGAGATCCAGGTTAGGTGAGAGAGATCATTCCTTCACGCAGTCCCATGACAG
GTTGGAACATTTCAAATAAGGTCTGTTCTGCTCCTCTGGTTGAAGGGAGAAAATTGGGAACCGG
TTTCCCACCTTCTACAAACCAAGATCTCATGTTGCCACGGGAGTGGCAGGGCAAGTGCAAGTGA
AAATGCCATACAATTTTCTACCATTTTGAACGCGGGTTTTTCTTCAATGGTCATTTGCTTGGTT
GCTGTAGGCCTTTCACTGTTTTCCAGAGCTCCCATAAGATTACTTTAGCCAGTTTTTTGTTCTT
TCCTGATGCTTCCCTGGCAGAGTAAGGGTTGGAACTTCCACCATTTTGCTGATTCATAACTCTG
TAGTCAGTTTTAAATATATTGATACTTGAGTTTGTTTTATGGCCCAGAATATGGTCTTGGTAAA
TGTTTCACATATACTCAGAAAGAATGTGTATTCTGATGTTGTTACATGGGCTGTTCTATAAATG
TTACTTAAGGTGGTTAATAATGTTGCTCAAGTCTTCTATATTCTTGCTGATTTTCTTTTATTTA
TTTATTTTATTTTTTTTATTATACTTTTAAGTTCTAGGGTACATGTGCACAACATGCAGGTTTG
TTATATATGTATACATGTGCCATGTTGGTGTGCTGCATCCATTAACTCATCATTTACATTAGGT
ATATCTCCTAATGCTGTCCCTCCCTGCTCCCCCCACCCCATGACAGGCCCCAGTGTGTGATGTT
CCCCTTTCCTGTGTCCAAGCGTTCTCATTGTTCAATTCCCACCTATGAGTGAGAACTTGCGGTG
TTTGTTTTTTTGTCCTTGTGATAGTTTGCTGAGAATGATGGTTTCCAGCTTCATCCATGTCCCT
ACAAAGGACGTGAACTCATCCTTTTTTATGACTGCATAGTATTCCATGGTGTATATGTGCTACA
TTTTCTTAATCCAGTCTATCATTGATGGACATTTGGGTTGGTTCCAAGTCTTTGCTATTGTGAA
CAGTGCTGCAATGAACATACGTGTGCATGTGTCTTTATAGCAGCATGATTTATAATCCTTTGAG
TATATACTCAGTAATGGGATGGGTGGGTCAAATGGTATTTCTAGTTCTAGATCTTGAGGAATCA
CCACACTGTCTTCCACAATGGTTGAACTAGTTTACAGTCCCACCAACAGTGTAAAAGTGTTCCT
ATTTCTTCACATCCTCTCCAGCACCTGTTGTTTCCTGACTTTTTAATGATCGCCATTCTAACTG
GTGTGAGATGGGATCTCATTGTGGTTTTGATCTGCATTTTTCTGATGGCCAGTGATGATGAGCA
TTTTTTCATGTGTCTTTGGCTGCATAAATGTCTTCTTTTGAGAAGTGTCTGTTCATATCCTTTG
CCCACTTTTTGATGGGGTTGTTTTGTTCTTGTATATTTGTTTGAGTTCTTTGTAGATTCTGGAT
ATTAGCCCTTTGTCAGATGAGGAGATTGCAAAAATTTTCTCCCATTCTGTAGGTTGGCTGTTCA
CTCTGATGGTAGTTTCTTTTGCTGTGCAGAAGCTCTTTAGTTTAATGAGACCCCATTTGTCAAT
TTTGGCTTTTGTTGCCATTGCTTTTGGTGTTTTAGACATGAAGTCCTCGCCCATGCCTATGTCC
TGAATGGTATTGCCTAGGTTTTCTTCTAGGGTTTTTTATGGTTTTAGGTCTAACATTTAAGTCT
TTAATCCATCTTGAATTAATTTTTGTATAAGGTGTAAGGAAGGGATCCAGTTTCAGCTTTTTAC
ATATGGCTAGCCAGTTTTCCCAGCACCATTTATTAAATAGGGAATCCTTTCCCCATTTCTTGTT
TTTGTCAGGTTTGTCAAAGATCAGATGGTTGTAGATGTGTGGTATTATTTCTGAGGGCTCTGTT
CTGTTCCATTGGTTTATATCTGTTTTGGTACCAGTACCATGCTGTTTTGGTTACTGTAGCCTCG
TAGTATAGTTTGAAGTCAGGTAGTATGATGCCTCCAGATTTGTCCTTTTGGCTTAGGATTGTCT
TGGCAATACAGGCTCTTTTTTGGTTCCATATGAATTTTAAAGTAGTTTTTTCCAATTCTGTGAA
GGAAGTCATTGGTAACTTAATGGGGATGGCATTGAATCTATAAATTACCTTGGGCAGTATGGCC
ATTTTCACGATACTGATTCTTCCTATCCATGAGCACGGAATGTTCTTCCATTTGTTTGTGTCCT
CTTCTATTTCGTTGAGCAGTGGTTTGTATTTCTGCTTGAAGAGGTCCTTCACGTCCCTTGTAAG
TTGGATTCCTAGGTATTTTGTTCTCTTTGACGCAACTGTGAATGGGAGTTCACTCATGATTTGG
CTCTCTGTTAGTCTGTTACTGGTGTATAAGAATGCTTGTGATTTTTGCACATTGATTTTGTATC
CTGAGACTTTGCTGAAGTTGCTTATCAGCTTAAGGAGATTTTGGGCTGAGACGATGGGGTTTTC
TAAATATACAATCATGTCATCTGCAAACAGGGACAATTTGACTTCCTCTTTTCCTAATTGAATA
CCCTTTATTTCTTTCTCCTGCCTGATTGCCCTGGCCAGAACTTCCAACACTATGTTGAATAGGA
GCGGTGAGAGAGGGCATTCCTGTCTTGTGCCAGTTTTCAAAGGGAATGCTTCCAGTTTTTGCCC
ATTCAGTATGACATTGGCTGTGGGTTTGTCATAAATAGCTCTTATTATTTTGAGATATGTCCCA
TCAATACCTAATTTATTGAGAGTTTTTAGCATGAAGGGCTGCTGAATTTTGTCGAAGGCCTTTT
CTGCATCTATTGAGATAAACATATGGTTTTTGTCTTTGGTTCTGTTTATATGATGGATTATGTT
TATTGATTTGTGTATGTTGAACCAGCCTTGCAACCCAGGGATGAAGCCCACTTGATCATGGTGG
ATAAGCTTTTTGATGTGCTGCTGGATTCAGTTTGCCAGTATTTTACTGAGGATTTTTGCATCAA
TGTTCATCAGGGAAATTGGTCTAAAATTCTCTTTTTTTGTTGTGTCTCTGCCAGGCTTTGGTAT
CAGGATGATGCTGGCCTCATAAAATGAGTTAGGGAGGATTCTCTCTTTTTCCTATTTACTGTAC
ATTTATTCCACCAGTGACTAAAACAGGTGTATCAATAAAATCTGTTCCCTCAGGTTTTGCTTCA
GGTATTTTGAGGGTCTGTTATCAGGTGCATAAACAAGATTGTTATGTCCTATTCTTAAATTAAT
CTCCTTATAATTATGAAGTTAATTTTTTTTTTCTTGAGATGCAGTTTTGCTCTGTCGCCCAGGC
TGGAGTGCAGTGGCACAATCTCGGCTCAGTGCAACCTCTGCCTCCTGGGTTCAAGCATTTCTTT
GCCTCAGCCTCCCGAGTAGCTGGGGTTACAGGTACCTGCCACCACGCCCGGCTAATTTTTTTGT
ATTTTTAGTAGAGATGGGGTTTCACCATCTTGGCCAAGCTGGTCTTGAACTCCTGAACTCTTGA
TCCACCCACCTTGGCCTCCCAAGGTGCTGGGATTACAGGTGTGAGCCACTGCGCCTGGACCTGG
CCCGAAGTAAACTTCTTTACCCTTGCTAATGATCTTTGCTCTGAAGCATGCTTTGCTGGTATTA
ATATAGTCATTCCTTCTTTCTTTGATTCATGTTTGCAGGGTATATCTGTTTCCATTCTTTTACT
TTTAACCTATTGTCTTTATATTTAAAGTGCATTTCTTGTAAGTATAATTGGTTTCTTAAAATCC
AATTATCTGCCTTTTAAATGTCATTTTTATATGATTTGCATAAATATGATTATTATTACAGCTA
AATTGAAATCTGTCATCTTGCTATTTGGTTTCTATTTATCCCATTTTTTTCCCCTCTTTTTTTG
CTTTCCTTGAGATTGAACATTGTATTAGTTTTCTAGGGTTGCTGTAAGAAAGTGACATAAAGTG
GATGGCTTAAAACAACAGAAATTTATTGTTTCAGTTTGGAGGCTAGTCATCTGAAACCAAGGTG
TCATCAGGGCCGTATTCTCTCTGAAACCTGTAGGGAAGAATTCTTTCCTGCCTGTTCTAGCTTC
TAGCATTTTCCAGCAATTCTTGGCATTCCTTTGCTTGTAGATGTATCCCTCCAATCTCTGCCTC
TATCATAACATAGCCATCTTCTCCCTTTATCTGTCTATTCTTCTCATCTTATAAGAACATTAAT
TACTGAATTGGGGCCCAGCATAGATTAGGCCTAATCTCATCTTGAATAGGTTACATCTGCCAAA
GATTCTTCTTCCAAATAAGATCACTTTTACAGCTTTTACAGGTACTGAGAGTTAGAACCTCAAT
ATATCCTTTGGTGGGGACCGACCTCTTACCCATAAAAAGTATTTTATATGATTCCATTTTATCT
CCTTTTTAGGTTATTAACTACAATTTTTTTTTCTTTTTTTTGAGATGGAGTTTTGCTCTTGTAG
CCCAGGCTGGAGCGCGATTTTGGCTCACTGAAACCTCTGCCTCCCGGGTTCAAGTGATTGTCCT
GCCTCAGCCTCCTGAGTAGCTGGGATTACAGGCATGTGCCACCGTGCCTGGCCAATTTTTGTAT
TTTTAGTAGAAACAGGGTTTCACCATGTTGGCTAGGGTGGGTCTCAAATTCCTGACCTTAGGTG
ATCCACCCACCTCGGCCTCCCAAAGTGCTGGGATTACAGGCGAGAGCCACCACGCCTGGCTTTA
TAATTTTTTTTTAATTCAGTGGATGTTTTAGGGTTTATAGTATACATCTTTATCACAGTCTAGC
TCCAAGTGATATATCCCTTTATGTATAGTACATGACCCTTACAGTAGTGCATTTCCATTTTTCC
TCTCTGGCATTTAGGCTATTGCACACACATACACTCAATTCATCCCTTTGTGTAGATCCGTATT
TCCAGCTGCTATCTTTTGCTTCTGTCTGAAATATATGTATGATTTCTTTTATGACTATTTTGAG
AAATTTGGTTATGTGCATTTGTCTATTTTTCTTATGTACTTTTAGCTCATCAATCTTAAGTCTG
TGAGTTTATAGTTTTTAAAAACAAATTTGAAATTATTTGGCTATTATTTCCTCAAATATTTTTT
TCTGCCGCCCCTGCTTTCCCTTTCCTTAGGGATCTCTGATTTCCACCTATATTACTCTGATTGA
AGTTGTTCCACGTCTCTTTTGAAAATCTCTGAAAAATCTTTTATCATTTGGATAATCTGTATTT
GTACATCTTCAAGTACATTAATATTTTCTTTTGCAATGTTTAATCTGCTGTTAATCCCATGTAG
TGGATTCTTCATCTCAGGTATTGCTGTTTTAATCTCTAGAAATTCCATTGAAGTCTTACTTGAT
GAAAAAGGCAGATAAAAACAAATGTTGGCAAAGATATGGAGAAATCAGAATCTGCACACACTGA
TGATGGGAATGTAAAATGGTCAAGGCAATTTGGAAAGCAGTCTGGCAGTTTCTCAAAAGGCTAA
ACGTAGTTCCCATATGACAGCAATCATTCATCTAGGTATATACTCCAGAGAAATAAAAACATAT
CCACACCAGAACTTGAACATTAATTTTCATAGCAACATTATTCCTAGGGGTTTAAAATTTTTTA
CATTATTTTTATTTAAAATAGAGACAGGGTCTCACTACGTTGCATAGGCTGGTCTGGAACTCCT
GGAATTAAGCATTCCTCCTGCCTTTGTCCTGTTTTCTCCACTGGAAAAGAATAAAACTTTGTAC
ACTTGGACTAACAACTCCCGATTCCCTCCTTTACCAACATGCCCCACAGCCTCTAGTAACTTAC
TCTCTACTTTCATGAATTCAACTTTTTTAAGATTCCACATATAAGTGAGATCATACAATATTTG
TCTTTCTGTGCCTGGCTTATTTCTCTTAGCATAATGTCCTCCAGATTCATACATGTTGTCTTAA
ATGACAGGATTTACCTTCCTTTAAAGGCTGACTAGTACTTCATTGTGTATATGTATCACATTTT
CTTTATCCACTTATCTGTTGATGGGCACTTAAATTGTTTCAATGTCTTGGCTACTGTAAGTAAT
GCTTCAATAAACATGGGAATGAAGATATCCCTTCAACATATTGATTTCTGTTCTTTTGGATAAT
ACTCAGAAGTGAGATTACTGGATCATATGGTTGTTCTATTTTTTTCAGAAACCTCCATACTGTT
TTTCATAGCGGCTGTACTAATTTACATTCCCACTAACAATGCATGAGTTCACTTTTCTGGACAT
CCTCCCCAACACTTGTTATCTTTCATCTTTTTCATAAAAGCCATTATATAATAGGTGTGAAGTG
ACATCTCACTGTGGTTTTGATTTGCATTACTCTAATAATTAGTGTGAGCATTTTTTTTTTTTCA
TGTACCGAATGTCTTTTGAGAAAGGTCTCTTCATTCCTTTGCCCATTTTAAAATCAGGTGGTTT
TCTTGCTCTTGAGTTGTTTGAGTTCCTTATGTATTTTAGATATTTACCCATTTCCAGATATATC
ATTTATATTTTTTCCTATTCTTTGAGTTCCCTCTTCACTGTGTTGTTTCCATTGCTGTGCAGGT
CTTTTATTTTGATGCCACCCCATTTGTCTATTTTTGCCATGCTTTTGCAGTCATATCCAAAAAA
ATCATTCCCAAGACCAATGCTGTGGAGATTTCCCCCTATGTTTTCTTCAGTAGGTGTACAGTTT
TAGGTCTTATATGTTAAGTTTTAAATCTATTTTTTTATATGGTGTAAATAAGGGTCTAATTTAA
TTCTTTTGCATGTGGATATCCAGTTTTCCCAACACCATTTATTGAAGACCCTGTCCTTTTATAC
TTTTCAGTATGCAGATCTTTTACCTCCTTAAATTTACACCTCAGTATTTAATATTTGTTGCTAT
TATGAGATTTTCATAATTTCCTTTTCAGATAGCTCATTAATAGTAGATGGAAACACTACTGATT
TCTGTAAGATGATTTTGTATTACGGAACTTTACTGAGTTTGTGTATCAGTTCTACCAGGTTTTA
GTTTTGTTCTGGTGGAGACATTACAGTTTTTTGTATATGGTTATGTCATCAGTAATTACAGATG
ATTTAACCTATTCCTTTCCTATTAGGATGCCTTTTTTTTCTTTCTCTTGTCCAACTGCTCTGGT
TAGGACTTCTAGTACTATGTCAAAAAGTGATGAGGGTCGTACATGGCCTCCATACCTAATCTGT
TGAGAGTTTTTACCATGAAACCAGGTTGAATTTTGTCAAATGCTTTTTCTGCATCCATTGAGAT
GATCATATGATTTGATTTACACCCTCCATTTTGTTATGTGGTATATCACACTTTTTGATGTGCA
TATGTTGAACCACCCTTGCATCCTAAGGATAAATCCCACTTCATCATGGTGAATCATTCTTTGT
ATTCGTGAATCCAGTTTGCTAATATATTGTTGAGGATTTTTGCATCCATGTTCATCAGGGATAT
TACTTTGTAAGTTTCTGTCCTTAAAGTGTCTTTCTCTGGCTTTAATAACAGTGTAACACTACCC
TTGTAAAATGAATTTAGAAGTATTCCCTCTGCTTCATTGTTTTGGAAAAGTTTGAGAATTTTTA
TTAGTTCTTTAAATGTCTGGTAAAATTCAGTAGTGAAGCTGCCTAATCCTGGGCTTTCCTTTGG
TGGGATACTTTTTATTACTGGCTCAATCTCTTTTCTTGTTATTGGCTTATTCAGATTTGTTTCT
TCATGATTCACTCTTTGTAGGTTGTATATGTCTAGGAATTTATTCATTTCTTTAGGTCATCCAA
TTTGATGGTGCATAACTTCATAGTAGTTTCTTATAATCCTTTGTATTTTGGTGATATCAGTAGT
AAATGTCTCCTCTTTCATTTCTGATCTTATTTGAGTACTCTTTTTTTCTCCTAGTCTAGGTAAG
AATTTGTTGATTTTATCTTTCAAAAAAAAAAAAAACCAACTCTTAGCAACTCTTAGTTTTGTTC
ATTTTTTTCCAGTCTTTATTTCAACTGTGATCTTTGTTACTTACTTCTTTATGCTAACTTTCGG
GCTTAGTCTGTTCTTTTCCTAGTTCCTTTAGGTGAAAAGTGAGATTGTGATCCTTCTTCTTTAT
TGGCGTAGGTTTGTATCGCTATAAATTTCCATTAGGACTGATTTTGCTGCATCACATAAGTTTT
GTTTCCATTTTCATTTGTCTCAAGGTAATTTTTTATTTACTTTTTGACTTCTTCTGTGAACTAT
TAGTTGTTTGGGAGCATATTGTTTAATTTCCACATATTGCTGTATTTTCCACCAGAATTGATTC
TTGTTCTTGATTTCTAGTTTCACGCCATTGTAATCAGAAAAGGGATTTGATATGATTTCTGTCC
ACTTAAACTTAAGATTAGTTTTGTGGACTAACATATATCCTGGAGAATGTTCCATGGGCATTTG
AGAACAAAATGTATTTTGCTGCTTCTGGATGGAATGTTTCATATATGCCTGTTAAGTCCGTTTG
GTCTAAAGTGTAATTGAAATCCATTGTTTCTTTATTGATTTTCTGTCTAGGTGATCAATCTGCC
CATGGTGAAAAGTAGAGTATTGAGGTCCCGTATTATAGTATTGCAGCCTATCTCCCTCTTCACA
TCATTTAAAAATTGCTTTATGTATTTAGGTGGGTCAATGTTGGGTGCATATACTTTTACAATTG
TTATGTCTTCTTGGTGAATTAATCCCTTTATCATTATATAACAAACTTCTTTCTTTTTATAGTA
TTGACTTAAAGTCTATTTTGTCTGATAGAAGTATAGCTACCCCTGCTCTCAATTTCCATTTATA
TAGAATATCTTTTTCCATCCCTTCACTTTCAGTCTATGTGTATGTTTAGTAGAAAAGTGAATCT
CTTGCCGGGCGCAGTGGCTCACTCCTGTAATCCCAGCACTTTGGGAGGCCAAGGCGGCGGGATC
ACCAGAGGTTGGGAGTTAGAGACCAGCCTGACCAACATGGAGAAAACCTGTCTCTATTAAAAAT
ACAAAATTAGCCAGGCGTGGTTGTGGGCCCTTGTAATCCCAGCTACTCGAGAGGCTGAGGCAGG
AGAATTGCTTAAACCCGGGAGGTGGAGGTTGCGGTGAGCTGAGATCATGCCATTGCACTCCAGC
CTTGACAATAGCAAAACTCCGTCTCAAAAAAAGAAAAAGAAAAAGAAAAAAAAGTGAGTCTCTT
ATAGGCAGTATGTGGTTGTGACTTAAAAAAAAAAAAAAATCCATTCTGTCATTCTATGTCTTTT
TGTTGGAGAATCTAATCCGTTTACATTCAAGGTAACTATTGGTAAGAAACTGCTAGTGTCATTT
TGTAATTTGTTTTCTGATTGTTTTGTAGGTCCCTTGTTTCTTTTTTCCCTATTGCTACCTTCCT
TTGTGGTTTGGTGGTTTTCTGTGGTGGTGTGCTTTGAATCCTTTCTTTTATAGTATATGCGATT
ACCATTGTATTTGCTGTAAGGCTAACTTAAAACATTTTATCCTTAGGCTACTTTAAGCGATAAA
AACTTGCCTTTAGTTGCATACAAAAACTCTACGCTTTCACAACCCCCCCGCCTTTCGTGATTTT
GATGTCAAAGTTTACACTTTTTTAAATTTGTATCTCTTATTGTAGCTACAGTTACAATTACTTC
TTGTAGCTACAACTTATTGTAGCTACAGTTGTTTTTAATAGTTTCATCTTTTAAACCTCCTAAT
AGGAATAACATTGCTTTACCTGCCACCTTTACAATACTAGAGAATTCTGACTATGAATTACTTA
TACCATTTAGTTTTTTACTTTTATGTTTCTCATTAACTAGCAGTCTTTTATTTAAGCCTAAAGA
ACTCCCTTTTAGTAATTCCAGTAGAGCAGGCCTAGTAGTGACAAACTCCCTTAGGCATTGTTTA
TCTGGAAAAGTGTTTATTTCTCCCTTTGCCAAGTAAAGTATTCTTAATTAACAGCTTTGTTTCC
TTCAGCACTTTGAATATACCATCCCACTCTCTACTGGCCTGTAAGGCTTTTGCTGATAAAGCCA
CTGAATCTGTATTGGGGCTACCTTGAATGTGATGTTTCTTATCTTTGCTGCTTTCAGTATTCTT
TGTCTTTGATTTTTGATAACTTGGTTGTGATGTGTCTTGGTGAACTCTTTAGGTTGAATCTGAT
CGGTGACCTCAGCTTCCTGTTCCTGAATTTTGTCATCTTTTCTCAGATTTGGGAATTTTTCAGC
TATTACTTCCTTAAATATGCTTTCTAGGCCTTTTTCTTTCTTTTCTCCTTAAGGACCTCCTATT
ATGCAAAAGTTAGCTAGCTTGATGTGTCCTGTAATTCTTATAGGCATTCTTTTTTGTTTTTGTT
TCTCATTGGATAATTTCAAATGTCTTACCTTTGAGCTCATTGATTCTTCTGCTTGATCAAGTCT
GCTGTTGAAGCGTTCTACTTAGATTTTCAGTTCAGTTACTGTATTCTTTATCTTTAGAATTTCT
ATTGTTTTTGTTTATATTTGTTAAACTTCTCATTCTGTTCAGATGTTGTTTTCCAAATTTCATT
TTTCTATCCATATTTTCTTGTACTTTGTTGAACTTAAGAGGATTAGTCTAAATTATTTGTCATT
TCACAGGTCTCCATTTCTTCTGAGTCTGTTACTGGAGCTTTCATATCCAATGTACACCAGAAAC
TTTGCTAATTCCACAGTAAATGTTAGAAATGGGTTTTTATTGATAAAATCAGTGGCTTGCTAAT
TAGTATGTTAGAAGCTGGCTTGCATTGGTGAAGTTGAGGGTATTCGATATACGTATCCCGGCTA
AAATACTTCTAAAGAACCTCTCCAGCTTTGCAAGAATAGAGTATAGGAATCTGAGGAGGCCTTT
AACATGGCCCTCAGAAAGGACACACAGGGCTTGACCTCTAAACATATAAACATATACAGAAACG
AGGTCAGAACATGTTTTGACTGATGCCAGTAGTAACACAGCAGAGCCTATTGCTGGTCACTGTT
ACCAATACCTTGTGTAATAAACCTTTCATTTAAATTGAGAGGTTGTAAGCCCCGAAAAAGCAAA
GGGCTTACAGTACCTCTTGCCTGAACACTCCTGTGTGTGCTTTCTAAGAAATGCTTTTAAAATA
ATGCCTGTGGCACAAATTGATTTGACACTGGCTCTCTTTATAAGCTGCTATCAAGGTTGTGGGG
AATAGCTAACTCTTACCCACTTTCCCTGACAAATAGGAACCCGTAGTTACCTACCAAAAGTATG
TAAAAGCTATATCCGACCCAAAGGCCAGTTAGGTCAAAACACCTTGAAAGGAGCTCAGTTTAAG
GAAATCAGTGGTGTCACAGTGCCCCACTAGCGTGTAAAAGTTTTCATAGTTTGACTATCGGATG
TGCTTAGTTTCACAGCTGAGTCTTACCTTGTGATACTTTTAGAGCAAAAGTCAAATCAAAGATG
ATTTAAAAAAACATTTCAAAGATTCATAAAGAATAAATGCCTCCTCAGGAAGTCTTCTGGAGTT
CTGCCCACTTCCCTTAGGTGGCTCTTTACTGTGCCCATCTCATAGCCTGTCTCCTTCCACTTGG
TGTATTGCAGAGTAAAGCTCACTGTTTACAGGGGTGGTAGAAAGTGTGGTCCTTTCCCAGACTC
CTTTTTCCCTTCCTCTTCTCATTTTTCAGAAGATGTGTTTGATAATAAACGAAACAAAATGACT
AACACATTGAGCTGAGCTACATAAGCAGATGTCAGTTTGACGTGAAGAGTTTAAAAGATCTATG
CATTATCTGGGGACTCCTCCCCCAGACCTGAAGGATCAGGTGCTGCCTTCTATGCCACCTGTGC
AGACAGCAAAAGAGGAAAACCATACCCACGTTCAGTATGAACAAAGGGGACATTTGAACTCTGT
GTGGACCCCTCATTGGAAGGGTGTTTCTTCTCCTGCTGCATCCACAAAGAGCACTCCTTAGCCT
TGCCTTTTGTCAGTTCTCTCTCCAATAAGGCTTGAGCAGAGACAACCCAGTGCAGTTCAGAGAG
ACTGAAGTCTGGTGTTCCAGGTCTGAGTCCTAGCTCTAGCTCTCTGTGTAACTTTGGGATGTCC
CAAAGTAACTTTTCACAACTTGATAGGTGTTAACTTGAATTTTGGATACAGGTGACTCTTAGCC
CATCCCCTCTCTGTGCTTCAGATATGTCATCACTTGGGCCATATGACCTCTGGACACCTTTCCT
ACTTTCCACAATTTCAGAGCAGCAGAGCAGACTGGAGCTCCTGCTGCCTCTGAGCTTCAGTGAA
TTATCACTCGTTGGAGGGAAGCTTCAAGCATTTTGTTATCTTTCAAGAGCAAACACAGTGTCTG
TCAGCAAGAATATGTAGCAGATGCTAGTGAACAGCAGTGATTAGGGTTGAATGCTGGATTTAAA
TATGGAGCTTAGGCTGTGAAGGAAGCCTGAAGAACCTAGAGCCCCATGAAGCTGCCCTCTGTGA
GATCTGACACCATTTCCCACTGATGAGCCATGGGTCTAATGGGTAGCACTGATTCCACATACAG
TATGTGAGTGCAATACAGTGAAAGCAAAGAGAATAAAATGATGGCTAACATGATGTTCCAAACT
TTAAACAGGAGAAAAACACACAATTCCATTATGTATAAGAACCCACACAGAGATCAGGAGAATA
ACCTCATTGGAGAATGAATGACCTGTGTGGGGAATTTAGGGTAGAGTTGAGATTGAAAAAATGG
GCCGAAGTCAGGTGGCCAAGGGCCTTAATACCTTGTATACAAGATGTGTAGTCAAGGAAGACCA
TGCCTTACTTATGCATCAATTCCCTTGGGCCTACAAAGAGCTGCCTAGCCTGGGACTGTTGTAG
AGAAAAGCTACAGTGTTCCAATGACACAGGGACTCCTCCATGTATGTATGAGTGCCCAGCTGGC
TCTGTAATAAATCTTATTTTTATTTATTAACTTTTCTTGCGCATTGGCTTGATGCATCAGTTGG
AAGTCAGAGGCCAAACGAAGTGAACACTGAGCCAAAGGAAGTTCTCAGCCTTTAGGGAGGCAGA
ATTCACTTTAAACACAATAAACAAATGAACTCACATTATACAGGAGAGAGTCAGAAGATCCCAG
TGGCTGGTGTCATCGGGCCATATTTGCCCGAAGTGCCTATTCCTTATAGGAACCCACTCCCAGG
GTTGATGGGCTACATCCTTAGGAGGCTTTATGCCTATGTTCTCCTGACCACTGGCTCCTCCAGG
GCTGGCCTTTTTTAGTCTCTCTGTAGAGGTTCCTGTAGCTGGTTGGATATAGGCTTTCACAGAA
GGGTCAGTGCCTTGGGTCTGGTTACAGGACTGTTAATCTTGCTTTGTTAAGAGTAATGTTATTT
CCCCATTTCCAAATTCTCCAGGGAGATGGAATGTCAAAGATAGTATGACTGTAGCACCTAAATC
CTGGGTTCCAGAAGCAGAGAAGAGAATACACTGAAGCTGTAGAAAGGCCTATTAGTCCATGTCA
GAGATTAACTGGATGCGAGGACCATTTCTGGGATGGTGTATACAGAACTGGAGAACTGGATAGG
GAGTCAGAAACAAAGAGCTGAAGATGATGCCTTTGAAGTTTCCAACGTGGATAACTAGGTCAAC
AGAATATTACTCAAGAAAGTATTAACATAGGATTGAGATGCAGTCAGTAGTAATGGAATTGAAA
TTTCAAAGTATATACCTCATGGATCTCAGGGGGTGTTGAGCTGATCACCTGGGCTAACACTCCT
ATGACCCTGGGGAAAATCAAATGACCTGGTACTGTAGCCATGGTAGGGGTGTCATCACCTTAAT
CCAACTGGGACAGTGCTGTTTGATATTCATCTGGAACTTGGTGCAGACCCACATTTTGCTGGGT
TTCACCACAACCAAGGCTTTTTTGATTCTTTTCTCTTTTAACATCAGTACATCACTGCAAAGTT
AATCCTCATATAATAGGAGATGAAACTAATTGCTTATAAAAACAAGATTTTTACAACACTAAAA
TTGTTCAAGCATATGGGCATATTTATAGTTGCAGGCAGTGTTTCAGATGCAGACTGTTCTTGGC
TGCAGTGGTTGTTTACAGGCAGCATCTGTTCTGATTAAATATTTGATGATTATCCCCGAATGTT
TTAAAGCATAGTACTGGGCTCTGCTGACTGTACAACAAACTGGCATTTTTGACCTATAGGGCAC
TGGGCTAGGAGATACAGTTCTGAGGGAAGTGAAAGATAGTTAACAACTGCACAACTGACCCTTT
ATTAGTTGCAATAAAGCAATCCAACCACCCAACCCACTGTGATGGCTTTCCTACTATTTAAGGT
TGGTGGTGTCAAAGAGACACCCTCCTGTACAGTGTGCAGTGAATCAACATCATTTCCACAAAAC
CTCCTTCCTGCACAAAGGAATTATCATACTTTGTTACGAAGTAAAATTTTCCTGTATCAGTCAC
AGGAGTTCACCAGTTAAGATACTGTTAGTTGAAGACTTCTGGGGTGACTTAATGAAATAGCTCA
GCCATCTGGTTTAAAAACTGGATTCTTCTATCCCTCCACACAGCTGTCCATGCACCTGCATCAT
CTCAAGGCTGGTCTCCCTGGTGGTAGAAAGGCTGACAGTAAAAACTGGAGCCACATGATTCCTT
GCTTAGGCAGTGTTTCTTCATACTCTCACATGAGAGCAGGCATGTTCTTTCCCTAGGCTCACAG
TAAACACTCTGTCAAATCTCACAGGCCCAAAGTGCTTTTGGTTATCCCCATTCCAAGCCAATCT
GTGGCATGGAAGATAGCATTACCCTGACTGCCTTAGACTAATATACCTACTCCACTTCTGGGGC
TGGGAATAATTTTGAGGTCAACCATCCAAACTGCATGACAGCCATTCCATGGAAGAGGTATGGC
CTAAATCTTTGGGGCAACCTGAATTCATGAAAACTCTCTTAGATTTATGTAACTATTTTTAGAA
TTCACTTCTGTATCATTTAATTTTACTAATAAAAATACCACCTTTACCCTAAATGTCAGCCAAG
CGTAAGGCTCCGTTGGGACAGAAGGAACTATCAAAGCTTTGTGTTTTTATACATTAGCAGCATT
TGACAAAGAAATAACTCTGAAGGAAGGAGAATAACCAGGCAGAGTCTAGATGCATGGAAAAGAA
GTCTTTGAGAAGGCTTCGCAGGCTGAAGGAAAGGGCAGGACTACTTCAGGAATACAACGTTTAA
GTAAAAGAGGTTGGGGTCTGTTGATCTTGAGGAGAGATGAGGATGGACTGGAAAATAGGAGTGA
GATATAGTAGGAGAGGAAAGGATATGGATGATGTATATACTGTATGGGTAACCATCAGTCGACC
CTAAGAAGATAATAGTTGTTAAATGGTTAGCTATTTAATGAAAGAAACCTGAACAAGTAATAAT
CTTGAGTTGCAAAGTGGCTGGAGTCACACAACAGACTAAATTTCTGGTAGAACAAATCCAGCAG
CTTATGTGAAGGTTACCATGTCTGAAGCTGGATAGAAAAGGTCTAACTTCCAACCAAAGTCACA
GTTCTTGAGCTCGGTACACAGAGACAGACTGCTAACAGCTGATGTGTCCTCAGCGAGAGTGTCC
TCATTTATATCCTCGTTCTCTTCTGCCTCCTTTTTTTGTTTTAAACTTTTGGGAAGTCTCATCA
TTCAATACAGTTCTAATATATCACAATAACAGAGGCGGCCCAATTTCTACAAATTGACTAATTC
TATCCCTGAAAAGTTCATACAATAAAACTATACAAAGCATCATTTTCAACCATCCTATAGAAAA
ATTTCCCTATTAAATTTTAACTCTAAATCCTCTATCTCTGTTAATAACCTTACTAAAAACTTCC
CCATCACTGCCTGCCAGGGAGATCAAAAGAAACCAAATTTAAGAAACCTCCAACACCTGTACCT
GACTGAAAAGCAAACATACAGACCTTTCAGTCCTGCCCCTACTATTCAGCTCCTATCACAGAGA
CATGGTGGATGTCCCCTTGGGAAGCGGATAGCTCTTAGGTGGAAGCTAGGCCTGCAATAAGCAG
GCCAGGAAACATGTCTCCCAGGCCCCATACTCTTGGCAGAAGCACTTGGCCACCAGACAGCATG
TCCTGTCAACCTACAGAGTTCTTAAAAACAAACACTGGGACCCAGAATAGTACCCTGTGGTCAT
AGTGCCCACAGTTCACTAAGCACCCTCACAGGTCTTTGACAGAACACTGACTGCCAGGTCACCT
GGTGGGCAGAGAAATGGAAGATTCCCAGGCCCAACTAGCATCTCAGGGAAGAACCACAAGCAGA
GCAACTTTCAGAGCTGGTCGGCCAGCGTTGGCACCCAGGGAAGCAAGTGCTATTCCATATTTGG
AGAGAACATAAAACTCAAGGAAACAGAGAAGTCCTACTCAATACCGTTCTCAACTGAAAACAAG
AGAACTTGCAAAAAAGAAACCCAGTTTTCTGGAGTCCATGGAGAAATATGAAGCCAATGCTCGG
CTAACAGAGCAGAAAGCCTTTTATAAATAATGCCAGTCAAAGCTCCAAAGGTCAGAGCTGATGC
ATGCCGGATTGTTCTGACAATTTCTTAGGTTTCTAGGCAACAAGGAGCTGGTCAAACAGCTCAC
CTCCAAGGACATACTTTATAATACCACCCTGGTAGACGAGAGCGAGGCAGCAGTGAAGGCAATA
GTCAAGAACAGGAGAGGGAGGTAAAGAACAAGATGCTCTTCAGACAGTCTGGACAAAGACAGTC
CCTACCCAGCTCTCAGGAGTTGCCTCCTTAAGAAGGTGGAGGCCCAGGCAGCTCCCAGGGTGAC
CCTAAAGACAGACTCCAAGGAAAAGGGTGTGAGGGCAATGGTGAATATAAGGAGGTCCCCTTTC
AGTAGGCAGAGCAAGTCAGTTCAGGTCTTATTTTTAGAGTCTCTGAACAGTGAAGAGAAGCTTT
CTGTGGACAGCATTCCACCACCATGGGAGGGGAAAGGTGCCATGAGAGACTTCTCCAGTGGGGC
ATACAAGCATTGTGCAGTGATCCCCAAGATCCAGCCACTGGCAGGAAATCGAAAGGCAAGCTTC
TTAAATACATAGTATATGGAGACAGAAGTGTGGAGCACTCCCAAAATGAAAGGCCAAGACCCAG
GAACAACCTCCACAACCTGGAGTATATACAAATGAACCCAGCCCTGCTGACTCAGATCCACACC
ATCCTAAAGCAGGGGTTTCTCATGAATGAAAGGGCTAAGTATTTGGTGGAGAGAATGAGAAGTC
TCCCACTGCCCACACATTTTTTCTTCAGAGATTTCTTTTCCAAGAGCCCCTTGAATAAAGGAAG
GGAGGGAGCACTGAATGCCCCAGAAATCAGAATGCATGGTGCGGGAAGACGACAGGAAATAGTT
CTCAAAGAGATCAGAAAATAAATTGGAAGCAATTTCATTACCACAAAGAACAGACATTTTTTCT
AAGGCACCCCTCCCCTTTCCACCCAATTGTCATTCAGCAACTACTGAATACTTACCAATGAAAA
ACGTTACCCCTGACCTCAAGGCTGCTCCTGAGCTCTGGTAGAAAATGCTTTCCTTGTCTATAAA
ACATGGCAAGCAAGGCAGGATTTAACAGTAAGGGCACAGTAGCACTGCAAGCCTTCAAATGGAA
ACCTGGAGACAAGCAGTGAGAACAGGAAGCAAAAGCAGGGCAGGTGAAGCTAGGACCAGGGCAT
CTGGAACTTTCCACACAGGTTGGATCTCCATGCCAGACAACAGTTTTCAAGGAAAAATATCTAA
GAGGAACATGACTTTGGGAAACTTTTTGGCAGTACTGCTTACTGTATACTAGAGAGTAAAAGAA
TTTGGGGAACATTCACCAATTTGCTTCTTCAGGGGCTTGGGTAGGGAACGTGAACAGGAACCTG
GCTCTAATTTCTGAACTTTTTTATCAGTAAAAACAATCCAACAAACGAAAGCTAGTCAGTGAGA
GAACTGGGAGGGTCTGCCCTCCTTCCCTGAGTCAAGCCTTCTGGGGGGACCTCCTGACATTTAA
TTAAGCAAAGACAACGCCCACTGAAGGAAGCTGACCTGAAAGTGACACGCTACTGTGAAATGAG
CATGAAGTGGGAGCTTGTTACATATATGAAATGGCCAGCGATCCTGAGCAAAGCGCTTCAGAGC
TTGAGACCTAAGTCTTCTCATCTATATACTGAGGGCTGGACAAGATGATCTGTCAAGCCATTTT
TATCCCTAATCCACCAAAATCCAATGCTTTAGTTTATTGTCACAAAAGCAGGTATCGAATGGCT
ATCCTGCAGTGCCTCCAATCAACATTCAGACTTTTTCCCTGAGGCAATATAAGATAACAGTTAA
CATGTTTTTATCAATTAGGTGGTCATGAGATAAATATATATGGGAAGTGGTAGTTTTTCACTTA
AATGCATATAATAATGGTACAGCTCTCTTTGAATAGTATTTGTTTATTTCTTAAATATTTAAGT
TCCTAAAGACGGTAAGAATAACCCAAGGAAGTGAAATCAATGTCACAAAGCACATGGCTAAATA
ACTGCAGGTTTGCAGTGCCATGTGTGAGATCAGATGACAGAAGGGAGAACTACCTTTAGGCAGA
GGCTTCTCATGTCCCCTGGAGTGGCCATGTGCTGTTCTACATGACTACTTCCACTTCGGTTATG
TAGAAGCTATTTAAAGCACACAGATGTTTGTGATGAGAAAAAAGCCACCCTTAATTGAATAATG
GAAATTATAAGCATGATTTGAGGGTGGGGGTGGAGGTGGGAGTAGAGATGGGTAGAAAGGAGTG
CAATGGAAACAAAGGAGCCTTCATAAAATTCAAGTCACTTCTTAGGATAACGTGATTGATTTAC
TCACCACCTTCTTAGGAACATAAAGCAAACAAGTGGGTTTTCCTTTTACTGCTTTTCTGAAATG
AGCTACACTCAAGAAAGCAGCACGGGGGTTGTGCTGTCCCTGCACAGTGGCAGGAGAGTATGAG
GAGCAGGTGAATGCCACAACAGCTCCATCCAAGATCATTTTTCACATGCAGGAACCATTCTTAT
ACTACCCTTTACTGGTAATTTCTGTAGAAATCTGGAAGTCTGGTTGACACCCTCCTGTACAGTG
TGCAGTGAATCAACATCATTTCCCTTGGACTTTGCAATCACGGTGGCATTCATACATTCATTCA
ACAAGTATGTATGTACAGGACAGTGGAGTAAAGAAAACAGATAGTTCTTACTCTCACGAGGCTT
AAAATTTCAGGAGGGAACTAGGCAGTCATGAAGTAAACATAAAAACACAGATTGTAATAGGCAC
TAGAGAATAATGAAGACTTTGGGGAACACAATTTAAATTGGAAAAATATCTCAGTTCCGTTAAC
ACATGTTGAAGGGGAATAGCATTGTTTTGCTGGATTTGGGGCCTGGATGCAAGCATGTTGGGTA
TGCATTGTAGGGTAATGCATTTCCTTCCATTTGGGCCCAAGTGTATATTTACCACCCAGTTGTG
ATGAGCTGGGATCCTCCTGCTCAATCTCAGCTTGAAGCACTTGGAGGTTATCTGCCTGCTGTGG
GTGATTATTTTGGAGCAAGGTACTTCATTTGCCTCAAGAAACAGATTTGATACCACTACTGTGC
CCTTTTGGAACAGAGAAGTAGGCAAGACCCCAGTGTGAGGCAGAGTGATGGGATCTTTAGGGAC
ATAATTGATGATGTAACTGATGATGATTTTGGAGTTTATACATTTCCAAAGTTTCAAAATATTT
TCACTTGGTTGATTTATCTTTATGGTGATGACACTACGTAGATATATGCCCTTCTTAAAAGTTA
CAGTAAGAGGCTGGGAGCGGTGGCTCACGCCTATAATCCCAGCACTTTGGAAGGCCGAGGTGGG
CAGATCATGAGGTCAGGAGATTGAGACCATTCTGGCTAACACGGTGAAACTCCGTCTCTACTAA
AAATACAAAAAAATTAGCCGGGCGTGGTGGCGGGCGCCTGTAGTCCCAGCTACTCAGGAAGCTG
AGGCAGGAGAATGGCGTGAACCCGGGAGGCGGAGCTTGCAGTGAGCTGAGATTCCGCCACTGCA
CTCCAGCCAGGGCAATGAGCGAGACTCCGTCTCAAAAAAAAAAAAAAAAAAAAAAAAAAAGTTA
CAGTGAGAGTTGACATTGAGAAAAGGGAGGCCCAGCCAGGGTTATGCAAAGACACAGTAGGGAG
GAGGATGGGAAGTTTCTGAGACACCCCAGTAACTGCATGGGTCCACAAAGCACATGTACAGTCT
GCTCTATGCACTGCAGGTACAGCCACGAATAAGACAAAGTCTCTGCCCTCATGGAGCTTGGTGT
CTGTCTACTGGAGCAGACAAAAACAAACCTGCTTTTTCTCATTTGCCTCTACTGTGGGTTCCGT
GTATAACTCTCAAATGCTAGCATTTCCATGCATTCCATCCTTGTCCTTCTCTCCTCTCTGCCTT
TTCCAGGAGAATTTTCCTAGCCACAGGTTCAGCTATGGTCTATGTGCTGGAGTCATACATTTTT
ATCTTCTGTGTACGTTTTTCTCTAGACCCATATTTTTAATTGCCTTAGGACAGCTCTCCCTGGA
TATCCCTCAGACACAACTAAAACATCATTCAATTGTACTATTAATAATTTTCCCTTCAAAATCC
ACTCCTCTTCTCTATAGACCTAACAGCAACACTGTCCGTCCAGCTCTCAAAACCTGAAATGTGG
GAGTTGTCTTTGACTCTTATCTTTCCCATGCATGAGCACTGAATCAATCAGAGTCCCTAGCATG
TCTTGGACTGTGGCCTCCCTTCTATCTTCAGATTATCTGCCTTTTTAGAATTATGGCAATAACT
TAACTGCAAAGACCACATGTGCACTTGTGCTCTTTCAGTTTGTAATAAATATAAAATGAAAAGT
GATTATGTCTTTGCTTAACGTCTGCAAAATAAAAGTCCAAAGTCCTTGGTATGGTCTATAAGGC
CCTCATTATCTGACCTGCCTGCCTTCCCAATCTCATCTTAATCCCTTACAGCCTGACACTCAGA
CATACTAGACTTTCCACCTAACTCACTCACATACACCTATCCTGTATGTCAAGATTCGGCTTGA
CCTTCATCACCTACCTATCTGCAGTATTTTTGGATCTGAATTCTCTGCCCACATTGTACTTTCA
CATACTTCTTTTACCCTTAGTGGTTTGTACTTATGCCTGGTCTAACTAGATTGTCTCCCTGTAA
CAGACTTCTTGATTCAACAAAGCAGCTCTGAATCAGCCTGAAACCTATGGCACACTGCAAAATG
GCAAACATTCAATAGGTATTTGCCCAGTAAATGTTAATGAAAGGAAAAAAATTCAAACTTCAGT
TGGAATAGGATTAGAGACAAGTTAAAAAAAAGTTTCCCATAGAAATCTTCCTCATCGTAAATAT
CATCATCCTAAATGTCCCGAGTCTTTCCATGGGTCTAATTTACCAAATCATGAAGACTCTCTTT
CTCACTGTGTATGTGTAGTGGGAGAGGCAGAGACAGAACGGTTTTTTTTTTTTTTTGCAAGTCT
GCTCTCTGGATTCGTTTTTCTGGGGATGAAATCTCAGGCTATGTAGCTTTTCTGGTCCCTTTTT
GGTAATCAACAAATCATCAGTTTCTCTAGGTATTAAAAAGCCTACACTTTTGAAACACCAAGAG
GCCAAACTCTCTCTTAATTGAAAACATAATTCTGCCTCTTACTGAGGTCTTTGGGTGGGAAGCT
TAAAGACAGCAGTGTTGGGGCAATTGGTTTTTCTTTTCCTCCCTCCACCTTCTTTCCCATTCAA
GAGCTGTTGCTGCCCTTTCTGGAGGGGAGGGAATTAGAAAAAAAGATCCTGCCTTACCCAGTCA
CTGTTAATTATTACTTTGAGCCAGGGTGGGGAATGACTTTCTTTCCTGGAATACACCCTGCTGG
AAACCACAGCTGAGATTCTCTAAGCTGGCAGCTTCCAACCTCTCCTCCCTCAACAGCTTCAACA
TTATATGGCACTCAGCTGCAGGGTGCCTGGCTCCAGGCCGGCAGTCCATTTGTGCAGGGTAGTT
TTCAAGATGGCTCATAGCCCATCTCTTTCAGTGACCAGCGTGGCCTATGGGAAACATACATCTT
GACTCCATTATTGGTAGGAGTACTCTTTAGTTAACTGCCACAGGCCAGTTCAGACACGTCTTAC
CCTGAGAGCCTTTTCAGAGTCAGGTACCATCCCTGTGTCCCCCAGCATCTGAAGATCACAGGTG
AAGGTCTCCAAGTAGTTCACTTAAAAAATACCTCAGTAGGTTTAAGAACAGGGAAAGCTCCCAA
TTCATTCTGTCAGGAGTATGACTCTCATCCCCAAACTGGACAGATATATAATATGAAAACTACA
GACCAATATTCCTTATGAATATAGATGCAAACATTCTAAACAAAATACTAGCAAACCAAATCCA
GCAGCATAGAACAAGGTTTATGTAGCATGGACAACTGAGATTATCCCAGGAATGCAAAGTTAAT
TCAATATACAAAAGTCCATTAATACAACATATTAATAAAGGACAAAACAACATGAAAATCTTGA
TACAGAAGAAGCATTTAACAAAACCCACAGCCCCTCCTTTTTTTTTTTTTGATTAAAAAACACT
CAGTAAACTAGGTTTAATAAAAGAATTTCCTCAACTTGAAGCAAACCCACAGCTAGCTAACATC
ATACTCAATGGTGAAAGACTGAATGCTTTCCCCTTAAAATCAGGAACAAGAAAAAGATGTCTGC
TCTTGTCACTTCTATCCAATATTGTACTGGAGGTGCTAGCCAGCACACTAAGGCAAGAAAATGA
AATAATAGGAATCTAGACTGGAAAGGAAGAAGTAAAAGTATTTCTGTTCACAGATGCATGATCT
CATATGCAAAAAATACTAAGTGACAAAAAAAGAATTTTAGAAAAGCTACAATATACAAAATCAA
TATACAAAAATTAATTTTATTTCTAACACCAGTGATGAATACAAAAATAAAAATTAGAAAATAA
TCATATAATTAAAGTGGCATCAGTAAAATACTTAGAAAAAAATTAAAGACGTCAAGACTTGCAC
ACTGAAATCTCTAAAACATCACTGAAATAAAGACCTAAGCAAATGCAAAGACATCCCACAGTCA
TGGAACAGAAAACCTAACACCATTAAAATAGCAGTTATTTCTCAAATTTATCTACCAATTCAAC
TAAATCCCTATCAAGATCCCAGCTGTTTTGCAGGAATTGACACAATGATCATATAAAAATTCAT
ATGCAATGCAAGGGACCCAAAACAGACAAAATGATTTTGGGGAAAAAAAAAAAAAAAATGGAGG
ACTTGCACATCCCAAATCTAAACTTACTACCAAGCTACAGCCATCAAGACAGTGCGGTGCTGGT
ATAACGACAGACATATAGGGCAATGGAGTAAGACTGAGAATCCAGAAAGTCTTATATTTATGGT
CAACTGTTCTTTGACAAGGGTACCAGGACCATTCATGGGGAAATAATAGTCTTTTCAACAACTG
GTGCTTGGGCAGATGGATATACAGATACCAATGCACTTATGCAAAAAATGGATGAAATAGGAAA
CTTCACTACATTCTACTGCATGCTCGGTCATTTTCAATCATTTAGGTGGCAACACTGACAAGAT
AACAGAAAGATGGAGGTAATAACATGTGAAAGGCAAAATGGTTTGTTTTTTTTTTTAAAAATGA
CAGTCTCTATCATGAATTTACTTACACTCCAGGCAAAGGTTATTAGAAGAAAAAAAGATGTAAG
AAAATTCCTTAACTGAAATGTGGAAAGAGTATCAAGAGGAGACCCTAAGACACTCTGTAAGAAT
CCCAGTGACTCCTCACTGTTCAACTAAGAAATGTACCCCATTATGCTGTGCTACCACGGAAGCA
TTGGAGGCACTTTGGGGGTTGATGAAGTCTTCATGGATGAGGTACTTTAAATATCTGGTCATGA
AGAGTATTTGAGAAATATGACAAGTGAAGATGCTGGGTAGGAATGCAGCGAGGAAAGTGTGTAA
CACAAGGCCAAATTGGAAAGGTCAGTGAGGGGGCAATCTGTGGAGGCACTGAATGCAGAGGATA
TTAAAATTAGCAAGATAGTGTTCTAGCACAATGAAAAGGATTGGAAGAGGGAGATGAGAGTCAG
GGAGTGAAATTAGGCAGCTGCTAAGAATGTCCGGGTGAGTCAGAGGATCAGGACCTGTAAGGGC
AGTATAAAGAAGGAAGGAATGATGGGAGAGGTTTAGGGGGAAAGAAGTGACAACCTGTGACAAT
TGAGGAATGAGAGATTTTGATGATTGGGGTGAAGGTTACATTTCTTAAAAAGAAACAAAGAATG
GTCGGTCGATAGAGATGCAAGATGAAGAATTTTGTTTTTAGGACTACTGACTATGAGGTAACAA
AGAAATCCCAGAGACAGAGGTCTGAGCAAAAGATATGGGATTGGGGAAGAATCTTTGGGAGTGA
CTGAGGTTGACTAGAGGGAACTGGGCAAGAACAGGTAAGGACTTTAAGCAGAATTGGAGGAGTA
TCCATCTAAAATCTGGGTAAACTGGGATGGAAAAACAAGTAGCCAGAGAAGCAACAGCCCAAGC
TAGGGTGTTGTCAAGGTTATAGATGTTACTGATTTTGGCAATGAGGAGATTATAAGGATCCTTG
AAGACTGTACTTTCGGTGAAACTACCATGCATTAGTAGCCTTTCACAGTGATATCTACCCCACA
CCTGCATCAAAATCTTGCAGTGCCTATTAATAAATGCCAACCCACTAACGGGGAGGGGAGAAAA
GACTTGGGACTCTGTACTTGTAAAACCCTCTACCCTCCCCAGGCAATTCTTTACACACTAACAC
TGTTGAGGTAAAAGGAGACAAGTGAGGGAGCAAATGGAAGGTGTGTTTTTGGATTATACAGTGG
CTCATGGAAGGGTGGGAGGGGTACAGATGGCCCTTAGACACTGGCGGAAAGTCAGAGAAAAAAA
ATTACAGTAAGCAGGTAAAGGGATCAAGACTACACAGGGACTAGTCTTGGGACGACATTTCTTC
CTCTGACAGTTTGTATGGAATTCTTGAGAAAAATTCCTCGAAGGGGCCTGAAATCTCAGAATGG
TCATGTTTTTAATGGGAATAGGGAGTGATGCTGCCCCATTACAACCATCTGTTCTAACAGAATG
TCTGTACCGAGGAGGGATGAGTAACATCGGCAAGTTCTGTTCGAAGCCTTTTTCAAGTTTCTTT
TTGATATGTATCTATCTATCTATCATCTCCCTATACAAGCAAGCATCCCCAAAAGTAGTTGTCT
CAGGAAACCAGGGTTAGGATGACCAGCTCATTTGCTGGAGCTGCCAAGGTCCAGAAAAGTTTGG
CTGCAGGCTGTTTTCATGTTTTATGTATGTTTGAAATGTTCTATAATAATAAAAGGTTAAAAAA
GTTTACATTTATTTGGAAAACCAGTTACTTTAGTTTATGGTTCCTTTTTTTCCCTCCAGAGCTT
CCTGGAGATTGAGGTCTAATTCAAAGAAAACCAAAATATATAATAGAGTACCTGGGCAAAAAAA
GTACTTTTATAACATAACATTTGGGGTAGAGGAAGTATCCACTGTAGTCAAAATGTCTATGTTT
TGCTCTTCCTTATTGTTCAGGGACATTCCATTAAATAGTAATGAAAAGGCAGCAAAAGTAAGAG
GAGTGACAACATGCCCGGCATAATTAAGCAAGCTAGAGCAGCTATTCTGTGCAACCGACGATTT
TTTTTCTCTAAAATTTTAAGGGTAGGTTCATTCTGACTCTGTTAAAAGTCTACTTGATGTGAAC
AACTCTATATCTGATAACCTATTTCAATTACCACTTTAAAACTTGTCATATGGATACGTTATTA
CAATTGTAGAACTTTAATAAATACCATAATAATAAAACTTGAGAACTGAAGAGCACACATTTCT
TCACGAATTTATTATATAAAACGCCCTCAGAGTATTTAATTTCTCCTCACTTTAATTACACATT
AAGAAGCACAGTGGATGAGAAGCCTTTAAGATGACTACAGTTGCACGAAGGTCCCTTTCATCAA
GGTAGCGTATGTACCCTAACAGTGTTCTAAAGGCTGGCCCAGAAAAACCCCATGTTACCTTATC
ACAATATGGAAAGCATTGTCTTCTTTTTCCACTAAATTAAATTATGGTGAAAAGTGCCACAGTT
TTATTTAGCATTATGGTACATAACAAACAGTTCTGTCTCAATTATGAAAAAAATTAATTAAAAT
AATCCTGAAAGACATCCTTTTTCTCCCCCCAATGATTTGAAAGCTGCATTTTTCCTGCCAATTT
CAAACAAACAAATCATCAGGTTGATCTACAGTAATCAGTTAAAACAATCAGTCAATCAATCAAT
CAATCACCAAGGCACAAGCTCAGCACATTAGCTATAGCTTGTAGCAAAAGGATATATCAATGTC
TCACCTTAGTTAAAAATACATAATCCTTTTATTTTATAATGCAATAAAAGAAATTAACAACATC
ACATACACAGAAGACTAGGAAAGGGGAAACTACTTACTTCTGGAAATCAGTAATGTAAACCTAC
TTGTACTTTTCCATAGTACATGAAAGTAACGTTTAACATGTTTTGAATTAATTAATTAAATTTA
ATCTGTGGGGCTATACAATGTAATTCTTAGGAGTAATAGTTTCATTCATTTCCAGGTCAGCTTA
CTGTATGATTAAGTAACACAAGGCACAGTAGCCATCTTTTTCATTATGTTGCAACACTGATCAC
GTGCCTCGATAAAATGGCTGATTCAACAAGATGATGGCAACACGAAGGGGAGACTTTGGATTGT
CTATTTAAAATCTAGGTAATAAGTAAGTAATTAATAAAAACTCTATCTTAAGTGCACTTTCACA
TGCTTTTTGTTTATAATAAACAAACAACAAACTTCCTAACTTTGTTGCAATAGGCTTGACTACC
ATTTCATTTGGCCAAATGCACTTTCCCCAGTAAACTTAAAACAACAACGAGAACAACAAGAACA
AAAATCCCTGTCCTTTCATATACTAAGAAAGAGGATTGGCTACTGAAACAGTTCATTGCAAGAC
ACATGAAGACGACATACTGTGGCATGAGTTGTTTTTGTTTTTAATTTGTTGTGCTGTTACTAAA
GTTCTGAGGGCTGCAGTTAAAACATTCCAATTTCTCCCTTCCTTCCATCTTTCTTTATTGATTG
ATTCTCAAGATTTTGCACAGAAAACTCTTTGGGGGCTAGAACAGCAGTAATTGCATCACACTGT
TTTCAAGACTTCAAGTTTCAAAAGCAAATCATTAAAAAAAATACAGTTCCTGATTTGAGTTAGA
TACAGGGACAAAAAAGTAGCACATACTTGAAGGTTACGTGGTCTACAAATGGTGGCAATATTTT
CCTTGGGAGAGTAGTTCTGTTGGTATATATTTTTTAAATACTCAAAAGGCTCAACCTCAAGCAG
TAATAAACACAAGCAAAAGTGATTTAACCCTTAAAATAAATATTCAGAAAAACCTCTCTGTACA
TACAAGTGAAAGAATATGTAACACTTTCACGCAAAAAAATAATTATAATAATAATAAAGGATTT
GTTCATATATGTAGCTGAAATCTGCTGTTCCAGCCCACATGTCCCCAATAAAGAAGGGAGGCAC
AGACATAGGTGACTACTGTGGTTGACTATCTTACAGCCTTTTTGTACTGGGACACTATCACCAC
CAAAAATTTATCCCTCGTTATATTTTTAAAATTTTTTAAATTTTTTCTTTTTTTTTCCTTCCTT
TTTTTTGTTTTATTTTGTTTTGTTTTGTTTTACAGCATGCCAAATCCTTTGGCATACGTGATGG
CCTTCAACAATCTCTCTTTAAGTTTTTCTTTGCTTGAGTATTCCGGAAGTAAAAGCACATTAAA
GCAAGTATGAGATGTAGGTAACCTAAATAGAGAAAAGGGGAAAAAAACAGGAAAACTGTAAGTC
ATGGGAAATACACTTAGAATTAAATGCTCCTATTTTTAGATTGTATATAGTTGAGACGGTCTGC
AATGCAAACTATACATTAATGCAAATCATAAACTTTTTGTTGTGTAACTACCAAGTTGCCTTTA
TCCTATAAATTACTCAAAGCTAGTGACGATGATAAGATACTGTATCCATTGAGTTTTTACTACA
TAACAGATACCATTTTAGGTACTGAATTCTTACAGTTCATTTAACTAAATCTTTCCACAACAAA
ACCACAGAGAGGACATCAGTAAATGCACTTTAGAGGTTAGGTCACTGAGAAGTCAAGTAACTTC
CTCTAAGGTGGAGAAAATACTCAAACCTGTTTTACAAGACTGCAAAGTGTGTGCTCTTAAATGC
TTATTAGAAACACTGCTGGCAATATGACTAAGAAAATGATTTGATAACAGGATTCTAGCACAAT
CAAATGATAATCTTCCGAGCCTCAATGTAACCATTCTAAATAGATGATCATGTTATATGGCTTT
CAATTAACAAGCTGGGAATCAAAAAAGTAAATGAATCACACTAATTTGATTCCAAACCAATGTG
AGCCCCATAATAATTTTTAACTAGGGCAATTTCTTAAAAGTTTCCTCACACAATGACAGCAAAG
TATTTTCTCAATTGTCTAATATGATTTGGGGATTTGTATATAAAATCACGAATGTGCTCAGAAA
CTATAAAGACAGTTCATATGTATGTGACGAGGAATGCAAGGTTTTCGGTAGGTATACAGTCACA
AGTTAATAATTACCTACCTTTCTGTGTCTGGGCCATTTTTGGCTATAATCATCTTTAATTTTCC
TAGTCCTCCCACAGGTGCTCTGTCTGTGCCCGTTGTAAACTGCAAGAAGAGTCTTTTCTGTTCA
TCTGTAAATGAATGAACGATTTCCCAGAACTCCCTAATGAGAAAAAATACAATACTGGTTTCAG
TTTGGCATTCATTATGACTGGTACTAACATAAGCTTATGATTTGCATTAAAACTATATTAAGAG
ACAACTTGGAAGTTAATTATCAGGATAGTATCACTTCTGGTGATTTAAAAATTTCCAAGCAAAT
TTATCCTGAACAGCTCTGAATACGTAAAAATTTAGATTAGATTACAATATAGTAAAATATTAGT
ACTACAATAGTAAAAAATTGAGAAAACCCAAGTGTGTAGTAACAGGAAGTGACTATTTAAACTA
TGGTATAATCACATTATGCAGTCAGTAATGAGCAAAAGACAAAATCCTATGAATTGAAAAAGAC
TGAAATGAATATTTGGAAAAATTAACTCCAGGTGCCTTTGGGTATATATTTTATTTATCCTTTC
TCCTTTATTCTCCTGGTCTACTCATCCTTTAACTCAACTTTGGGAGGAAAAAGTGATACAGATT
AAGGACAAAAGAAAAAAAGACCCCCTGCCCCAACCAACTGGCCCCAAATGCAAACAACTACATA
CCTAATAGCAGTAATACAAAAGTTCAATTTTTATATGAACTAGAATACTTTAAACAAGTAATAT
GTGCTGTGTATGGAGGAGGAGAATTATTCTGACATTTCTGCCACCTGCAGTTATTTTAAGAGAA
TGATTTATCCTGTGGCATTCCTAAAATCTATGTAATAAAAGCTATGTTTTAATGACACTTATGT
TAGTTTGAGTTCTAAAAAACGAAATACAAGCTCATAAAGTGCAATCTTGAAGTTTATTTGAATA
ATCAGGCATCTATAAAACATATATACACTAGTTCATAGCTAAATAAATTTTTTTTTTTTTGAGA
CAGAGTCTTGCTCTGTCGCTCAGGCTGGAGTGCAGTGGCGCGATCTCGGCTCATTGCAAGCTCC
GCCTCCTGGGTTCGCGCCATTGTCCTGCCTCAGCCTCCCAAGTGGCTGGGACTATAGGTGCCTG
CCACCACGCCTGGCTAATTTTCTGTACGTTTAAGTAGAGGCAGGATTTCACCATGTTAGCCAGG
ATGGGTCTCGATCTCCTGACCTCGTGATCCGCCCACCTTGGCCTCCCAAAGTGCTGGGATTGCA
GGCATGAGCCACCGCGCCTGGCCTATAGCTAAATAATGTTAAGATTAAAAAATTAAAAAAAAAA
TTAAAAGTATTTTTAGTTGCTTATATAATATAAAATGCATTTTAATAGTTATTTAGAAAGTTCT
TTCCAGAGCCAGGTGTGGTGGCTCATGCCTGTAATCCCAGCACTTTGGGAGGCTGAGGCCAGTG
GATCACCTGAGGTCAGGGGTTCAAGATCAGCCTGACCAACGTGGTGAAACCCTGTCTCTACTAA
AATACAAAAATTAGCCGGGCGTGGTGGCAGATGCCTGTAATCTCAGCTACTTGGGAGGCCAAGG
CAGAAGAATTGCTTGAACATGGGAGGCGGAGGCTGCAATGAGCTGAGATCACACCATTGCACTC
TAGCCTGGGAAACATTTTGAGCCATCTCAAAAAAAAAAAAAAATTTCATCTCAAAAAAAAAAAA
AAAAAAAATTCCAGAATGTTAGCAGAGAACATCACACATCCCAAACCAGTAATTACAATTTTCT
ACCTTTTGATATTCTAATATCCCAACATACTATTTTTATTACCAAAATGCTGGCATTTTTGGTG
CTGCAACACCATTTATCTGAAATAACTTAAAAGTTTTATTAGAATTCTTGGCTTTCTCCAATCT
CTTGCCACTTCCCTTCCCTGCCCCTTTACAAATCCACCAGTCAGCAAGAGAAAACAAAAAAGAA
ACACACAACAACAGAAAACTGGACATGAAACTTGCAGGAGCATCTCTCAGGTAAGGAGGAGAGG
AATCGGACCCCAGTTCATGATACTCCTTTTGGGGAGGCTGACAAGAGAAAACAAGTCTGCTGTA
GCACAGAACCCTCCAACATAGCAGAAAAGCTGCCCTGCAGTGGGTGGTAATAAAGCCTCCTCAC
ACTGCAGGAGGGCTCTGAAGCTTAACTGAACCAGCTACACCTAAGGAGGTAGACCAAAGAGCAC
CACCGAAGAACAGAGACAGACCCTGCAGGAAGTGGCAGATCAGCAGTGGTCACAATGACCAGTC
TGGCTATATTATTACGGAGCTGTTTCTTTGGACTTTTAAACGAAACCTTTAAAAATTTTATACA
ATGAAAACAGGGAACAAAATGCATCTCTATTCCTATAAGTGTTATGTGTGTTACATTAACATTT
TGAATTAAACAAGAATGCATATATTTAGAAAGCTGAAGAAAACACGAAGAAGCTTCCAGGTTTC
CACATAAAAGTGGTGGCTGTATCGATCACATCCTACTCACATCTCTAAAAATCTACCCGGATCA
AAAAGGGGAAAATAAAAAAAAAACCTATGACTTAGGTCTAGAGTAAAACTAGGAGACAGAACAA
TAAACTCCAAATACCAAAGAAGTGGGGAAATGAGCAGGGTCCAGCAGGAGCTACATCGAAGAGT
GGCTTGTGCGGAATCATACTGATTAGGGATCACCCAAGGCCAGACCCCACCCCCTCCACCACCT
GCCCCTCAACAGAGCACTACTGAGTGTAGGTTAGAGCACTGGCAACAGGGTGGTTAAAGGAAGG
ACTACAGAAAAGTACTTGGGTCCAGCAATGGCAGGCTCAGGAAGGCACCATGCATGAAAGGAAG
GGGGGTACCTTCAAAAGCAGAAGTGTCCCTTAAGCGGCTGTAAAGGGGTGGAAGCAGCTAATGA
AAAAGAGTCTTCATTAAGCTACATGGAGCTGAAAAATCAGAAAGTGGTGATTCTGCCCTTACTA
AAGCAACAGAAGAGGGATCCCTTCAACCAAGACCCCTAAAGCCACACAATTCCTCCCTGCTCCA
CCATGAGGTCTAGTAATAACAGGTCCAGAAAAAAGTAACATCTGTTATAGAAACATAAACAAGA
AAAACAGAATTCGGAAGTCTAAATAAAGTTATTATGGGAAGAGTCTGGCGAATGAGAAGCAAAA
CTTTCCGGTAGACAAAAGTAGACCAGAAAAATTCAGTCATAAACAATGGGAAACTAGTAACACC
ACATTCCAACACAAAAGGAGAATGCAGCAGCAGACAAGACAGACCACCAGTGATGAGAAATACA
AATTCAGAAAGAAATGAGCACATCTTAAATAAAAATAAGAATACTAAAAAATACTGAAGAAATA
TGCAAGGTAACTTTGGAAATAAAGGCTAATTTTATTATAAGGTGACCAAAGAAGAAGCAGTATG
ACTAAAATTTCTACTGAGGACAGACTCTAGAAAATTAAAAAACAAAGAAAATGAATAAAATAAA
CATAACTAAAGAAATGCATATCAAAGCATAATAACGAAAAAGATTAATAGCTAACTACATAAAC
ATAAAACCTTTCATTTGGCAAAAAAATCCTATAAGCAAGGTTAAAAGACAAATGACAAACTGGG
AAAAAATATTTGCGAATTTACCAGCGTTAAAGGACTAATCTCCTTAACATATAAAGTTTCTAAA
AGTGAAGAAAACGACCAGCTGAGCAGCAAAATGAGCAAAAGACTATATAAAACTATAAACAGCT
CTACAAGAAAAAATGTCCATTATCATTCATAATTGAGGTAAGCTCAAAAATTGTAACTATAGTA
AAATACCATTTCTCAATTGGCAAATAAGTAAACATCCACATTTAACAACACATTCTGTTTGGAA
AGTTTTAAGAAAAGAGATACTGTTGTAAACTGCTGGTGGGAATGCAAAATGTTATTTTTCTGTG
AAGGAGGATTTGGCAGTATCTAGCAAAATTACACATGCATCTACCATTTGATGCAACAATCCCA
CTTCTAACAATTTATCTTAATGATACTTTACATATGTACGGAATAATGCATGAACATTAAGAGG
ATTCACTGTGACATTAGTACTAACAGCAAAAGGTTAAAAATAACCTCGATGCCCATAAATATTG
ATAAGCTATGCTGCATCCACACAAAGGAGCAGTATTCAGTCATACAAAACAAAATGGAAAACAC
CTCTGCAATAATGTGGGATGATTCTCAGGATACACTAGGAAGATTATTTTCCTAGTTCTGTCCT
CTGATAAGGCCTACAAGCAGTAACGTTCAAAAGCAAAGGCCATACTTTGCATCTAAAATCTGAC
TTCTAAATGCCATTCTCCAACAATTTATTGGAAAAATAACTTATTCCAGGCCTGAGAATGAATG
TTCGAGATTAGTTAGAAATCTCAAAATCTAATAGGGTCATTTTAAAAGCACACGATAGCTAAGC
AATTTGAATATCATTTAGAGTAATGACTGTGAAACGTATTAAATACAAAAACATTCATTAGTTC
GTAATATTCAAAAAGGCAGCAAAAACCAACCAAAAAACAAAATTAAATTGTCACTATTAAAATT
ATTACATTAACTCCTTACTCTAAAAACCTTAAATCTATTTTATCATGCCTTTTCTGTAGAACTG
TTTTTCAAGGTAATCAAATGGCACCCAATGATGAGAAAAAAGAATGCCAGGTATATATGTAGGA
CAAGCAGATGGAAATTTTTTTTCCCCAAACAGCCATTTTGCAATCCCCAATGAGATAACAGATT
AAGGTAATGATACTGATAAATACGAAAATCAGGTGAAGGGCAGGTAGCAGGTTCTGGAAAGATG
ATGATGGCAACGACATAGTTATTATTATTATTATTTTTTGTTTTATTCCCTCCCAACCTCCCCT
ACAAAAACAGCAAACTGAATGAGAAAACCAAAGACCCAGAGACATTATCTACAACAAAACCATG
AACCATTGCATATAATTGGACAGAAACAAAGCTCTGACAACTACAAGACTGGTTAGTAAGGAAG
CAGATGCAAGGTAACTGACTGGGTTTCTGACAGCCCTGAGAACACTGCCAACCCACTGGAAAGC
ACAGGCCAATCTGAAAACAGGGCTTAAAGTTTTAAAAAGTTCTACAGGATCTAATTTGCAGATG
ATTACAAAGGGATCATGATGTAGTAAGCTCTGGGCCCTTAAAACACCCGAATACCAAACCCCCA
CCAGAAACAAACCTTGTACCGAGGAAAAACTTCTGGGAATAACTTCCGAATGGACCAGGTCAGT
AATAAAAACCAAAAAAAAGTTCAAATATATGTGTGGGATAGAGGAGTAAAGAAGGCAGTTTCAG
AAAGCACAAGGGCATATTTTTGACCATTTTACAAAAACACAGACTTCTCCTCGTCCCTAAAAAG
CGAAAAAAGTTATCCTGGCCCATCTCTCCCTCGTCCAAGTATGAGAAACTCATTTCACTCACAC
ACACACACACACACACACAAATAAATAAATAACAGAACAGAGTGAAACAGTGTAATTATTAGAG
AAAAAGTATGTGCATATACATGAGAATGGTGTCCCTACAGAAAATCAAAGTACATGAAACAATA
TGCAAATAAAACATGAAAACTGTAAACAAATTTCAAACTGAGCTAAAAAGAATTTAAAAAATAA
CAAATCATTAGAAATGGGAAATTTCTGAATGAGGGTAACTGAAAAAAAGGAAGACATGACACAA
CTAAGGAGTAATTAGAAATGCAAGGAAAAAAATCCCATCAAATACAAAGAAACTAAAACTAAAC
TAGAAGAAACATAAAGGTGAATAAAACAGAACAATAATACCAGAACCTGAAGGCAGAAGAAAAA
TCTTAAAATCAAAAAGAAACAAACCCATGCTTGAGACTCTCCTTGCTGGTCCAGAGCCACAGTG
CTGCTGTGTACTGGCAGGAGGAATTCTGCTATAATTGGTCCTGGCAGTGTTCACCTTTCCTGCA
AGTGTTCCACAGCCCAGGGACACAATGTGGTCAGGAGCACTGCCAGGAACACCAGCAAGGGGGA
GCCTGCCACAACAGGCACAAGGCTCAGGAAGCACTCTCCAGCCCACGAAAATCTAGTGGGGGTC
CTCTTCCCTCACCCAAACACACTCTGCGCAGCT
SEQ ID NO: 2
shRNA artificial/synthetic sequence.
5′-TGCTCTTCTTTCTACTTTATTCTCGAGAATAAAGTAGAAAGAAGAGCA-3′
SEQ ID NO: 3
551 Ribo 1 artificial/synthetic sequence.
5′-GAAATAATAATCTGATGAGGCCGAAAGGCCGAAACTAAAAG-3′
SEQ ID NO: 4
551 Ribo 2 artificial/synthetic sequence.
5′-GAAAGAAGCTGATGAGGCCGAAAGGCCGAAAAATAATAATG-3′
SEQ ID NO: 5
UBE3A-ATS artificial/synthetic target sequence.
5′-TGCTCTTCTTTCTACTTTATT-3′
SEQ ID NO: 6
UBE3A-ATS artificial/synthetic sequence.
5′-CTTTTAGTCATTATTATTTC-3′
SEQ ID NO: 7
UBE3A-ATS artificial/synthetic sequence.
5′-CATTATTATTTCCTTCTTTC-3′
SEQ ID NO: 8
microRNA artificial/synthetic sequence.
5′-TCCTGAATAAAGTAGAGAAGAGCAGTCAGTCAGTGGCCAAAACTGCTCTTCTT
TCTACTTTATTCAG-3′
SEQ ID NOs: 9-508
UBE3A-ATS artificial/synthetic sequence.

| | |
|---|---|
| 9 | TGTGGAGAATCATTGATATAT |
| 10 | GTGGAGAATCATTGATATATT |
| 11 | GTATATGAGAGTTCCATTATT |
| 12 | TGGCTGTGGAAACGCTTATTT |
| 13 | TGGATCGATGATGAGAATAAT |
| 14 | GGATCGATGATGAGAATAATT |
| 15 | GCCCTCCAATAGGACAAATAA |
| 16 | TGACCCAAGACTTGCTTTAAT |
| 17 | GACCCAAGACTTGCTTTAATT |
| 18 | TGTGCTGAAAGAAGGAAATAT |
| 19 | ATATGGCATGCCTCTATTAAA |
| 20 | TATGGCATGCCTCTATTAAAT |
| 21 | ATGGCATGCCTCTATTAAATA |
| 22 | TGGCATGCCTCTATTAAATAA |
| 23 | GGCATGCCTCTATTAAATAAT |
| 24 | GACAGTGGAACCAAGTTTATT |
| 25 | GAGACTCCATGGTTCATAATA |
| 26 | AGACTCCATGGTTCATAATAT |
| 27 | TTGTGGTCATACTACATTATA |
| 28 | TGTGGTCATACTACATTATAT |
| 29 | GTGGTCATACTACATTATATT |
| 30 | TTGCAACAAGGTAAGTTATAT |
| 31 | TGCAACAAGGTAAGTTATATA |
| 32 | TTGATTGATGCCCTCATAATA |
| 33 | TGAAGTTGGGTAAAGTATAAA |
| 34 | GTCAATACACAACTGATAAAT |
| 35 | CAAAGAGTTTCAGCCATAAAT |
| 36 | CATGCTTTCCAGAGGAAATAT |
| 37 | TCCAGAGGAAATATGTTTAAT |
| 38 | TTTAATCATCTGCCCTATATT |
| 39 | TTAATCATCTGCCCTATATTA |
| 40 | TGCAAGATACCATACATTTAT |
| 41 | GCAAGATACCATACATTTATA |
| 42 | GGTCCATGTGATTTCTTTAAT |
| 43 | TGCCATAACTATAGCATTTAT |
| 44 | GGATACCATCCACACTATAAA |
| 45 | TGTGTTTCCTTCCAGATAATT |
| 46 | GTGTTTCCTTCCAGATAATTT |
| 47 | GTGTGCTGAAAGGGCTATAAA |
| 48 | TTGAACCAACCACCCTATAAA |
| 49 | TAAACGTTGTATGGCTTATTT |
| 50 | ATCCCTGTGTACACAATATTT |
| 51 | ATGTGTGGTAAATCCATTATT |
| 52 | TGTGTGGTAAATCCATTATTT |
| 53 | ATGAGCATTTGGCAGTAATAT |
| 54 | TGAGCATTTGGCAGTAATATT |
| 55 | GAGCATTTGGCAGTAATATTA |
| 56 | AGCATTTGGCAGTAATATTAT |
| 57 | GCATTTGGCAGTAATATTATT |
| 58 | GAGACTCTGCTGAAGTTTATA |
| 59 | AGACTCTGCTGAAGTTTATAA |
| 60 | CAGTTATAGAAGTGCTAATTT |
| 61 | TATGATGACCTCCTGAATTAT |
| 62 | GGGTCTGGTGTTTCATTTATT |
| 63 | GGTCTGGTGTTTCATTTATTT |
| 64 | GGGTCTCATTATAGTTAATAT |
| 65 | TAACAGCATGTCAGGTAATAA |
| 66 | GCAGAGCCCTATTCCTTTAAA |
| 67 | CTCCTAAATGTTTGGAAATTT |
| 68 | TTAGCATTTCTGACCTATTTA |
| 69 | TAGCATTTCTGACCTATTTAT |
| 70 | AGCATTTCTGACCTATTTATT |
| 71 | ACAGGATATAGGGAATAATTT |
| 72 | CAGGATATAGGGAATAATTTA |
| 73 | TAGCACTGAAATGCCTATATT |
| 74 | AGCACTGAAATGCCTATATTA |
| 75 | GAGCAGAAATCAATGAAATAT |
| 76 | ACTTGTAACCAGACCAATTTA |
| 77 | CTTGTAACCAGACCAATTTAA |
| 78 | ACTCAGTGAACAAGTAAATAA |
| 79 | TAGCCCTGTATCAAGTAAATT |
| 80 | ACAAGATTCCAGAACATTTAA |
| 81 | CAAGATTCCAGAACATTTAAA |
| 82 | GAGAGTTACCCAAAGAATTTA |
| 83 | AGAGTTACCCAAAGAATTTAA |
| 84 | AGAAGAAAGGTTTGGAAATTT |
| 85 | AGCCCAATCTATAGGATTTAT |
| 86 | GCCCAATCTATAGGATTTATA |
| 87 | CCCAATCTATAGGATTTATAT |
| 88 | CACATACTACAGGGCATATAA |
| 89 | ACATACTACAGGGCATATAAA |
| 90 | TTAAAGAGTTGCCACATTATA |
| 91 | GTTGCCACATTATACATAATA |
| 92 | AGACATTGAACCAGCTATTAA |
| 93 | GACATTGAACCAGCTATTAAA |
| 94 | AGGGTTAAAGAAAGGTATAAA |
| 95 | ATACCCGAGATGACCAATAAA |
| 96 | CAAATCCACGAGAAGAAATAA |
| 97 | ACTTGTGTTGATGGTATTATA |
| 98 | CTTGTGTTGATGGTATTATAT |
| 99 | ATTCATTGGAAAGGGTATAAA |
| 100 | CAGTAACAAGAGCCCATTATT |
| 101 | AGTAACAAGAGCCCATTATTT |
| 102 | GCCCAAGAGATCCACTTTATT |
| 103 | CCCAAGAGATCCACTTTATTT |
| 104 | TTTCATCTCACCCAGAATATT |
| 105 | TGTTATGAGATCCAGTTATTT |
| 106 | ACTGAACTGTGAGCCAATTAA |
| 107 | CTGAACTGTGAGCCAATTAAA |
| 108 | AGTCTTAGGTAGTTCTTTATA |
| 109 | CAAAGGCAGCAGCACAATAAT |
| 110 | AGACTGGCTATTTGAAATTAT |
| 111 | GACTGGCTATTTGAAATTATT |
| 112 | CACCATCAAGAGAACTAATAT |
| 113 | ACCATCAAGAGAACTAATATA |
| 114 | CCATCAAGAGAACTAATATAA |
| 115 | GGAAAGTACATAGTCAAATTT |
| 116 | CAATGCATACTACAGTATAAA |
| 117 | AGTCCTTACGTGTCAATAATT |
| 118 | GTCCTTACGTGTCAATAATTA |
| 119 | GACACATACAGGCTGAAATTA |
| 120 | ACACATACAGGCTGAAATTAA |
| 121 | CACATACAGGCTGAAATTAAA |
| 122 | CAATATTGAAGCACCTAAATA |
| 123 | ATGGATCTAACAGACATTATA |
| 124 | CATTCTCCAGGATAGATTATA |
| 125 | ATTCTCCAGGATAGATTATAT |
| 126 | CAAGATGGAAACTGGAAATTT |
| 127 | TTCTATGAGGTAAGCTTATAT |
| 128 | TCTATGAGGTAAGCTTATATT |
| 129 | ACAAAGATCAGACAGAAATAA |
| 130 | CAAAGATCAGACAGAAATAAA |
| 131 | ATACCACAGACATACAAATTA |
| 132 | TGAAGACTGCCAACCATTTAA |
| 133 | GAAGACTGCCAACCATTTAAA |
| 134 | CTAATTCAGCAACACATTATA |
| 135 | GATGCAGGATAGTTCAATATA |
| 136 | ATGCAGGATAGTTCAATATAA |
| 137 | TGCAGGATAGTTCAATATAAT |
| 138 | GTGTCTGTTGGCTGCATAAAT |
| 139 | CTTTGTAGATTCTGGATATTA |
| 140 | GCAGAAGCTCTTTAGTTTAAT |
| 141 | CAGAAGCTCTTTAGTTTAATT |
| 142 | CATCCTGTTACTGGGTATATA |
| 143 | GTATATACCCAGAGGATTATA |
| 144 | TATATACCCAGAGGATTATAA |
| 145 | ATATACCCAGAGGATTATAAA |
| 146 | TATACCCAGAGGATTATAAAT |
| 147 | CCCTAGAACTTAAAGTATAAT |
| 148 | TGACAAACCTGGAGGTAATAT |
| 149 | GACAAACCTGGAGGTAATATA |
| 150 | GTCCATTCTCACCACTTATAT |
| 151 | TCCATTCTCACCACTTATATT |
| 152 | CCATTCTCACCACTTATATTT |
| 153 | CATTCTCACCACTTATATTTA |
| 154 | GTAGATGACATGATCTTATAT |
| 155 | GAATAGAGAGCCCAGAAATAA |
| 156 | ATGCTTGACATCACTAATAAT |
| 157 | TACACTGTTGGTGTGAATTTA |
| 158 | ACACTGTTGGTGTGAATTTAA |
| 159 | CACTGTTGGTGTGAATTTAAA |
| 160 | GGTATTTGCACACTCATATTT |
| 161 | GTATTTGCACACTCATATTTA |
| 162 | ATAAAGAGAACGTGGTATATA |
| 163 | AGACACAGTGGAATCTATTTA |
| 164 | GGTACAAACTTTCAGTTATAA |
| 165 | GAAGGCACTGATATGTTAATT |
| 166 | AGGCACTGATATGTTAATTAT |
| 167 | ATCCATTCAAGCTAGATTATT |
| 168 | TCCATTCAAGCTAGATTATTT |
| 169 | GGCATGGTGGCCCTCAATTAT |
| 170 | GCATGGTGGCCCTCAATTATA |
| 171 | CATGGTGGCCCTCAATTATAT |
| 172 | ATGGTGGCCCTCAATTATATA |
| 173 | TGGTGGCCCTCAATTATATAT |
| 174 | GGTGGCCCTCAATTATATATA |
| 175 | GTGGCCCTCAATTATATATAT |
| 176 | TGGCCCTCAATTATATATATA |
| 177 | GGCCCTCAATTATATATATAA |
| 178 | ATGAACAACAATGGGATTTAA |
| 179 | TGAACAACAATGGGATTTAAA |
| 180 | GAACAACAATGGGATTTAAAT |
| 181 | AGCAATAGTGGAGAAATATAA |
| 182 | GACCTAAACCCTATCTTATAA |
| 183 | ACCTAAACCCTATCTTATAAT |
| 184 | CCTAAACCCTATCTTATAATT |
| 185 | CTAGAGCAGCAATACTAATAT |
| 186 | CTGAAGTGCACATGGAATATT |
| 187 | TCTTCAGGATGACACATATTA |
| 188 | CACAATGATATGGAGATTTAA |
| 189 | CTCAATCCAATAACCTAATTT |
| 190 | GTAAACAGAAGGAAGAAATAA |
| 191 | GATATCACCTTACAGAAATTA |
| 192 | AGGTCCGTATTACCCTTATAT |
| 193 | CATCTAGCCTTTCTGATTTAT |
| 194 | ATCTAGCCTTTCTGATTTATA |
| 195 | TCTAGCCTTTCTGATTTATAT |
| 196 | CTAGCCTTTCTGATTTATATA |
| 197 | AGTCACAACTCAGATTTATTT |
| 198 | GGCCTCACAACTCAGATTTAA |
| 199 | GAACAGAGCCCTCAGAAATAA |
| 200 | GGAAAGGATTCCCTATTTAAT |
| 201 | GAAAGGATTCCCTATTTAATA |
| 202 | GACAAACGGGATCTCATTAAA |
| 203 | CATCTGACAAAGGGCTAATAT |
| 204 | ACAATGAACTCAGACAAATTT |
| 205 | CAATGAACTCAGACAAATTTA |
| 206 | GTTAGAATGGCGATCATTAAA |
| 207 | CAGCCATCCCATTGCTATATA |
| 208 | AGCCATCCCATTGCTATATAT |
| 209 | GCCATCCCATTGCTATATATA |
| 210 | CCATCCCATTGCTATATATAT |
| 211 | CATCCCATTGCTATATATATA |
| 212 | ATATACCCAAAGGACTATAAA |
| 213 | TATACCCAAAGGACTATAAAT |
| 214 | ATATGCACAGGTATGTTTATT |
| 215 | GAAAGACACTGATTGTATTTA |
| 216 | ACATAGCCCTAACCATATAAA |
| 217 | CCTCACACAAAGACCTATTAT |
| 218 | TGGCAGAGATTTGGCAATTAT |
| 219 | GGCAGAGATTTGGCAATTATA |
| 220 | AGAACAACAAGGAGGAAATTT |
| 221 | GAACAACAAGGAGGAAATTTA |
| 222 | GAATGGAGTGAAATGTTTAAA |
| 223 | TCTGATTTCCTTGGGTTTATT |
| 224 | CTGATTTCCTTGGGTTTATTT |
| 225 | TGCTCTTCTTTCTACTTTATT |
| 226 | GTAAGCATTTAGTGCTATAAA |
| 227 | TTAGTCACATCCCACAAATTT |
| 228 | ATGGTCTGTGCTGTGAATATT |
| 229 | TTGAAGTCTCCAACCATAATT |
| 230 | CAGTTTGTGCATCACATATTT |
| 231 | TGCCCTCTTGGTGGCTTATTT |
| 232 | TCCTAGGTCATAATGATAATT |
| 233 | CTCCACATCCTTACCAATATT |
| 234 | CGCTTATCAGATATGATTTAT |
| 235 | GGTCTATACATGTAGATTATT |
| 236 | TCATAGATGTATGGGATTATT |
| 237 | CATAGATGTATGGGATTATTT |
| 238 | CTTGTAACTCCTTGGTTAAAT |
| 239 | TTGTAACTCCTTGGTTAAATT |
| 240 | TGTAACTCCTTGGTTAAATTT |
| 241 | GTAACTCCTTGGTTAAATTTA |
| 242 | TCCTTTCTGATGCTGTTTAAA |
| 243 | CCTTTCTGATGCTGTTTAAAT |
| 244 | CTGAAACTTTGCTGCATTTAT |
| 245 | TGAAACTTTGCTGCATTTATT |
| 246 | GAAACTTTGCTGCATTTATTA |
| 247 | TTGCTCTGTGAATAGATAATT |
| 248 | TGCTCTGTGAATAGATAATTT |
| 249 | ATATGTTGATCCACCTTTATA |
| 250 | TATGTTGATCCACCTTTATAT |
| 251 | ATGTTGATCCACCTTTATATT |
| 252 | TCTTTGGCTACTTTGTATAAT |
| 253 | CTTTGGCTACTTTGTATAATA |
| 254 | TAAGTGCATAGAGCCAATAAA |
| 255 | ATGCACATGATCTTCTTAATT |
| 256 | TGCACATGATCTTCTTAATTT |
| 257 | TGGCTTAAACAAGAGAAATTT |
| 258 | GGCTTAAACAAGAGAAATTTA |
| 259 | ATATGTTGATCCACCTTTATA |
| 260 | TATGTTGATCCACCTTTATAT |
| 261 | ATGTTGATCCACCTTTATATT |
| 262 | TCTTTGGCTACTTTGTATAAT |
| 263 | CTTTGGCTACTTTGTATAATA |
| 264 | TAAGTGCATAGAGCCAATAAA |
| 265 | ATGCACATGATCTTCTTAATT |
| 266 | TGCACATGATCTTCTTAATTT |
| 267 | TGGCTTAAACAAGAGAAATTT |
| 268 | GGCTTAAACAAGAGAAATTTA |
| 269 | AGGTATCAGAGTAGTATATTT |
| 270 | AGTTCATCGTTAGTGTTATAT |
| 271 | GTTCATCGTTAGTGTTATATA |
| 272 | ACCACCATGCCCAGCTAATTT |
| 273 | ATGACCCATTTGAAGTTAATT |
| 274 | TGACCCATTTGAAGTTAATTT |
| 275 | ATGCTGGCCTCACTGAATAAA |
| 276 | TGCTGGCCTCACTGAATAAAT |
| 277 | GCTGGCCTCACTGAATAAATT |
| 278 | TGTTCCCTCCTCTTCAATTAT |
| 279 | GTTCCCTCCTCTTCAATTATT |
| 280 | TTCCCTCCTCTTCAATTATTT |
| 281 | TTGGTAGGTTGTGCGTATTTA |
| 282 | ATTAGTTGGCATGCAATTATT |
| 283 | ATTCGTAGTTCTCTGAATAAT |
| 284 | TTCCTTCTGTTGGCCTTTAAT |
| 285 | TCCTTCTGTTGGCCTTTAATT |
| 286 | CCTTCTGTTGGCCTTTAATTT |
| 287 | GAAAGCATTTAGAGCTATAAA |
| 288 | CTTTCACTACCTGCCATAAAT |
| 289 | TTTCACTACCTGCCATAAATT |
| 290 | TTCACTACCTGCCATAAATTT |
| 291 | TCCTTGACCTATTGGTTATTT |
| 292 | CCTTGACCTATTGGTTATTTA |
| 293 | CTTGACCTATTGGTTATTTAA |
| 294 | TTGTGATCACAGAAGATATTT |
| 295 | TGTATAATCGCAGTCTATTAA |
| 296 | TCGCAGTCTATTAACATTTAT |
| 297 | CGCAGTCTATTAACATTTATT |
| 298 | GAGTGGTAAAGTCTCTATTAT |
| 299 | AGTGGTAAAGTCTCTATTATT |
| 300 | AGCATAAGCTATGTCATTAAA |
| 301 | CTCTTCATTTCCTTCAATATT |
| 302 | TGAGATACCTAGAACAATATA |
| 303 | GAGATACCTAGAACAATATAA |
| 304 | CTCTTTCTCTGTGAGATTATA |
| 305 | ACAACAGCCTGGAAGTATAAT |
| 306 | CAACAGCCTGGAAGTATAATT |
| 307 | ACAGCCTGGAAGTATAATTAA |
| 308 | ATTCAAACTGATGCCAATTTA |
| 309 | TTCAAACTGATGCCAATTTAA |
| 310 | AGTCAACACACCAATATTAAA |
| 311 | AGCTCCTGTTTGAAGTAAATT |
| 312 | GCTCCTGTTTGAAGTAAATTT |
| 313 | GCCTTCCAAGGTTTCTATTAA |
| 314 | TGTGGGTCTCTTTGGATTTAT |
| 315 | TATGGTTCTGTAGAGATATTT |
| 316 | TGTTCTCAATTTCCCTATATA |
| 317 | GTTCTCAATTTCCCTATATAA |
| 318 | AGGTTGGAACATTTCAAATAA |
| 319 | TTACATGGGCTGTTCTATAAA |
| 320 | TACATGGGCTGTTCTATAAAT |
| 321 | TGTTACTTAAGGTGGTTAATA |
| 322 | GTTACTTAAGGTGGTTAATAA |
| 323 | GTTGCTCAAGTCTTCTATATT |
| 324 | CAACATGCAGGTTTGTTATAT |
| 325 | ACATGCAGGTTTGTTATATAT |
| 326 | ACGTGTGCATGTGTCTTTATA |
| 327 | CTTTATAGCAGCATGATTTAT |
| 328 | TGTGTCTTTGGCTGCATAAAT |
| 329 | CTTTGTAGATTCTGGATATTA |
| 330 | GCAGAAGCTCTTTAGTTTAAT |
| 331 | TTTCCCAGCACCATTTATTAA |
| 332 | TTCCCAGCACCATTTATTAAA |
| 333 | TCCCAGCACCATTTATTAAAT |
| 334 | CCCAGCACCATTTATTAAATA |
| 335 | GTTGTAGATGTGTGGTATTAT |
| 336 | TTGTAGATGTGTGGTATTATT |
| 337 | TGTAGATGTGTGGTATTATTT |
| 338 | GTTCTGTTCCATTGGTTTATA |
| 339 | TTCTGTTCCATTGGTTTATAT |
| 340 | GGATGGCATTGAATCTATAAA |
| 341 | GATGGCATTGAATCTATAAAT |
| 342 | CCTAATTGAATACCCTTTATT |
| 343 | GGCTGTGGGTTTGTCATAAAT |
| 344 | GCTGTGGGTTTGTCATAAATA |
| 345 | TGTCCCATCAATACCTAATTT |
| 346 | GTCCCATCAATACCTAATTTA |
| 347 | TCCCATCAATACCTAATTTAT |
| 348 | CCCATCAATACCTAATTTATT |
| 349 | TTGTCTTTGGTTCTGTTTATA |
| 350 | TGTCTTTGGTTCTGTTTATAT |
| 351 | AGCATGCTTTGCTGGTATTAA |
| 352 | GCATGCTTTGCTGGTATTAAT |
| 353 | CATGCTTTGCTGGTATTAATA |
| 354 | ATGCTTTGCTGGTATTAATAT |
| 355 | TGCTTTGCTGGTATTAATATA |
| 356 | GAGAGTTAGAACCTCAATATA |
| 357 | AGAGTTAGAACCTCAATATAT |
| 358 | CCACCACGCCTGGCTTTATAA |
| 359 | CACCACGCCTGGCTTTATAAT |
| 360 | ACCACGCCTGGCTTTATAATT |
| 361 | CCACGCCTGGCTTTATAATTT |
| 362 | GTCTAGCTCCAAGTGATATAT |
| 363 | TTTGCTTCTGTCTGAAATATA |
| 364 | TTGCTTCTGTCTGAAATATAT |
| 365 | TCTTAAGTCTGTGAGTTTATA |
| 366 | TCTCTGATTTCCACCTATATT |
| 367 | CTCTGATTTCCACCTATATTA |
| 368 | GTATATACTCCAGAGAAATAA |
| 369 | CACCAGAACTTGAACATTAAT |
| 370 | ACCAGAACTTGAACATTAATT |
| 371 | CCAGAACTTGAACATTAATTT |
| 372 | GTCTGGAACTCCTGGAATTAA |
| 373 | CTTTCTGTGCCTGGCTTATTT |
| 374 | CAGGATTTACCTTCCTTTAAA |
| 375 | TCTGTTGATGGGCACTTAAAT |
| 376 | CTGTTGATGGGCACTTAAATT |
| 377 | TCATAGCGGCTGTACTAATTT |
| 378 | CATAGCGGCTGTACTAATTTA |
| 379 | TTTACCCATTTCCAGATATAT |
| 380 | GTGTAAATAAGGGTCTAATTT |
| 381 | TGGTTATGTCATCAGTAATTA |
| 382 | CCTTGCATCCTAAGGATAAAT |
| 383 | GTGAATCCAGTTTGCTAATAT |
| 384 | TGAATCCAGTTTGCTAATATA |
| 385 | GAATCCAGTTTGCTAATATAT |
| 386 | CCATGTTCATCAGGGATATTA |
| 387 | GTGTCTTTCTCTGGCTTTAAT |
| 388 | TGTCTTTCTCTGGCTTTAATA |
| 389 | GTCTTTCTCTGGCTTTAATAA |
| 390 | TTGTGATCCTTCTTCTTTATT |
| 391 | GTAGGTTTGTATCGCTATAAA |
| 392 | TAGGTTTGTATCGCTATAAAT |
| 393 | AGGTTTGTATCGCTATAAATT |
| 394 | GGTTTGTATCGCTATAAATTT |
| 395 | TCATTTGTCTCAAGGTAATTT |
| 396 | TTTGGGAGCATATTGTTTAAT |
| 397 | TTGGGAGCATATTGTTTAATT |
| 398 | TGGGAGCATATTGTTTAATTT |
| 399 | TGGATGGAATGTTTCATATAT |
| 400 | AGTATTGAGGTCCCGTATTAT |
| 401 | GTATTGAGGTCCCGTATTATA |
| 402 | CTCCCTCTTCACATCATTTAA |
| 403 | TCCCTCTTCACATCATTTAAA |
| 404 | TATGTCTTCTTGGTGAATTAA |
| 405 | ATGTCTTCTTGGTGAATTAAT |
| 406 | CTGCTCTCAATTTCCATTTAT |
| 407 | TGCTCTCAATTTCCATTTATA |
| 408 | GCTCTCAATTTCCATTTATAT |
| 409 | TCCTTCAGCACTTTGAATATA |
| 410 | TCAGCTATTACTTCCTTAAAT |
| 411 | CAGCTATTACTTCCTTAAATA |
| 412 | TCCTTAAGGACCTCCTATTAT |
| 413 | GCTTGACCTCTAAACATATAA |
| 414 | CTTGACCTCTAAACATATAAA |
| 415 | ACCAATACCTTGTGTAATAAA |
| 416 | TTGACACTGGCTCTCTTTATA |
| 417 | TGACACTGGCTCTCTTTATAA |
| 418 | CAGAAGATGTGTTTGATAATA |
| 419 | GTTTGACGTGAAGAGTTTAAA |
| 420 | CTCTGAGCTTCAGTGAATTAT |
| 421 | AGGGTTGAATGCTGGATTTAA |
| 422 | GGGTTGAATGCTGGATTTAAA |
| 423 | GGTTGAATGCTGGATTTAAAT |
| 424 | GTTGAATGCTGGATTTAAATA |
| 425 | AGTGAAAGCAAAGAGAATAAA |
| 426 | CATGATGTTCCAAACTTTAAA |
| 427 | AGGTGGCCAAGGGCCTTAATA |
| 428 | GCCCAGCTGGCTCTGTAATAA |
| 429 | CCCAGCTGGCTCTGTAATAAA |
| 430 | CCAGCTGGCTCTGTAATAAAT |
| 431 | TGGTGTCATCGGGCCATATTT |
| 432 | TAACTAGGTCAACAGAATATT |
| 433 | GCAAAGTTAATCCTCATATAA |
| 434 | GCAGCATCTGTTCTGATTAAA |
| 435 | CAGCATCTGTTCTGATTAAAT |
| 436 | AGCATCTGTTCTGATTAAATA |
| 437 | GCATCTGTTCTGATTAAATAT |
| 438 | CTGCACAACTGACCCTTTATT |
| 439 | TGCACAACTGACCCTTTATTA |
| 440 | TGATGGCTTTCCTACTATTTA |
| 441 | GATGGCTTTCCTACTATTTAA |
| 442 | AGCTCAGCCATCTGGTTTAAA |
| 443 | CTGACTGCCTTAGACTAATAT |
| 444 | TGACTGCCTTAGACTAATATA |
| 445 | CAGCATTTGACAAAGAAATAA |
| 446 | AGGATATGGATGATGTATATA |
| 447 | GTCGACCCTAAGAAGATAATA |
| 448 | AGAAACCTGAACAAGTAATAA |
| 449 | GAAACCTGAACAAGTAATAAT |
| 450 | AGTCACACAACAGACTAAATT |
| 451 | GTCACACAACAGACTAAATTT |
| 452 | AGCGAGAGTGTCCTCATTTAT |
| 453 | GCGAGAGTGTCCTCATTTATA |
| 454 | CGAGAGTGTCCTCATTTATAT |
| 455 | ATCCTCTATCTCTGTTAATAA |
| 456 | AGCAAGTGCTATTCCATATTT |
| 457 | CTGGAGTCCATGGAGAAATAT |
| 458 | CCTCCAAGGACATACTTTATA |
| 459 | CTCCAAGGACATACTTTATAA |
| 460 | TCCAAGGACATACTTTATAAT |
| 461 | CCAAGGACATACTTTATAATA |
| 462 | GTGAGGGCAATGGTGAATATA |
| 463 | TGAGGGCAATGGTGAATATAA |
| 464 | CGAAAGGCAAGCTTCTTAAAT |
| 465 | GAAAGGCAAGCTTCTTAAATA |
| 466 | GGCAAGCAAGGCAGGATTTAA |
| 467 | ACAGGAACCTGGCTCTAATTT |
| 468 | GGGACCTCCTGACATTTAATT |
| 469 | GGACCTCCTGACATTTAATTA |
| 470 | GACCTCCTGACATTTAATTAA |
| 471 | GTGGGAGCTTGTTACATATAT |
| 472 | CTAAGTCTTCTCATCTATATA |
| 473 | TTAGGTGGTCATGAGATAAAT |
| 474 | TAGGTGGTCATGAGATAAATA |
| 475 | AGGTGGTCATGAGATAAATAT |
| 476 | GGTGGTCATGAGATAAATATA |
| 477 | GTGGTCATGAGATAAATATAT |
| 478 | CACAAAGCACATGGCTAAATA |
| 479 | ACAAAGCACATGGCTAAATAA |
| 480 | CGGTTATGTAGAAGCTATTTA |
| 481 | GGTTATGTAGAAGCTATTTAA |
| 482 | AGCCACCCTTAATTGAATAAT |
| 483 | ACTACCCTTTACTGGTAATTT |
| 484 | TAATAGGCACTAGAGAATAAT |
| 485 | ATTTGGGCCCAAGTGTATATT |
| 486 | TTTGGGCCCAAGTGTATATTT |
| 487 | TTGGGCCCAAGTGTATATTTA |
| 488 | TGCCTGCTGTGGGTGATTATT |
| 489 | GCCTGCTGTGGGTGATTATTT |
| 490 | GGGATCTTTAGGGACATAATT |
| 491 | GATGACACTACGTAGATATAT |
| 492 | TGCTCTTTCAGTTTGTAATAA |
| 493 | GCTCTTTCAGTTTGTAATAAA |
| 494 | GTCTTTCCATGGGTCTAATTT |
| 495 | TCTTTCCATGGGTCTAATTTA |
| 496 | ATCAGTTTCTCTAGGTATTAA |
| 497 | TCAGTTTCTCTAGGTATTAAA |
| 498 | AGGCCAAACTCTCTCTTAATT |
| 499 | CTTACCCAGTCACTGTTAATT |
| 500 | TTACCCAGTCACTGTTAATTA |
| 501 | ACCCAGTCACTGTTAATTATT |
| 502 | CCCAGTCACTGTTAATTATTA |
| 503 | CTCAACAGCTTCAACATTATA |
| 504 | TCAACAGCTTCAACATTATAT |
| 505 | ATACATCTTGACTCCATTATT |
| 506 | CCCAAACTGGACAGATATATA |
| 507 | CCAAACTGGACAGATATATAA |
| 508 | CAAACTGGACAGATATATAAT |

SEQ ID NO: 509
Human UBE3A sequence.
CAATGAGAATAAACTCACTGTAGCTCCATGCTACAACATGGATACATCTCAAAAACAATTTTGA
TGAAAGAAGGTGGACACAAAAGAATACATGCTGTATGACTCCTTTTACATAATGTTCAAAAAAC
AAGAAAAACTAAACCATAGTATTTAGCAATTCAAACTTAGATGGCAAAAGTATAAAGAAATGTA
GGGAAATTATTTTCGTAAATGTCTCCTCTAGGGAAAAGGGAGGATTAAGTCAAGCTCATCCATC
ACTCCATACTGGCCCTTCCTCGCCAGTGATCTCACATGTTTATTCCAACAAGAGAGCTGGAGAA
ACTGTGTTTGTGCTATATCAACAATTCTCAGTGAATGTCTTGTTGACTGGGAAAGCAATGTGGG
TCCTACTTTTCTTTCTTTCATTTTGTAAAACCATTTCTGATGAGCATGAATGAGTCCTCCATTT
CTTAAGTAACTGACAGCACAGGTTTGATATCAGCAATCAAAGATCTGCCAGGCCTTGTTTATCA
TGCCTCACCTCTGTTGCCACATGTTTCCCAGGGATCGATTGAATGTGACTATGAGTAGCTCTCC
ATGTACACCATCTAGTGTCACTTCTGGGACTTGGGGGTTTCTGACTTACAGTCCTCTTCCCTAT
CATTTATTTACATATTCAGTAAATAATCATGAACACCTACCTTATGCCAGACAGTGTTCTGGGT
GCTAAATATATAGTAGTAGAAGAGATAAAGTTTATGTTTTCATAGAACTTTCAATTCAGTACAT
GAGATGGAAAGGTGGACGGTGCCTGTAGTCCCAGCTACCCGGGAGGCTGAAGTGAGAGGACTGC
TTGAGCCCAGGAATTTGAGACCAGCCTGGACAACGTAGTGAGACCCTGTCTCTATTAAAGTTTT
TTTAAATTACTGATATAATTTAAGGTAGTGGTGAGTACTAGAAAAAATGTACCAGGAGAAAAAA
ACAGAAGCAAAAGTACATGTGGCCAATACTATTTTGATATGTAGAGGCCAGGAAAAGCTTCTCT
GAAGAGGTAAACACAAAGTTGAAGGATGTACCATCACAGAGAGATGGAAGTTTTCCATAATTTC
CTTGCATATACTATGGTGTGACTTCATTTTTTCACCCACGTCTGTTAGGCTGCTCAGATAAACT
GCTTTTTTTTTAAACGAGATCTAAAGACCACGTACATTCTTCATAGTCCCTGAATTAAGTATTA
CCCAAACCATAGCTCATGGGCCACCTACCTGTTAATTTAATGTCTATGTATGCCTTCACTCTAC
AATGGCAGGCTTAAGCAGCTGGGACAGAGACCACATAACCCAGAAAGCCTAAAACTTTTATTCT
CTGGCCCTTTACAGCAGCAGTCCCTAAACTTTTTGGCACCAGGGACTGGTTTCCCAGAAGACAA
TTTTTCCTCCTACTGGGGGTGAGGGATGGTTTCAGGATGATTCAAGCGCATTACTCTTACTGTG
TACTTTATTTCTATTATTATTACATTGTAACATATAAGGAAATAATTATACAGTTCACCATAAT
GTAGAATCAGTGGGAGCCATGAGCTTGTTTTCCTGAAACTAGACAGTCCCATCTAGGGGTGATG
GGAGACGTGACAGATCATCAGGCATTAGATTCTCATAAGGAGTGTGCAACCTAGATCCCTTGCA
TGTGCAGTTCACAATAGGGTTCGTGCTCCTATGAGAATCTAATGCTGCCACTGATCTGACAGGA
GGCGGAGCTCAGTAATGTGAGTGATGGGCAGCAGCTGTAAATACAGATGAAACTTCCTTGACTT
CCCTGCCCTCGCCTCCTGCTGTGCAGCAATTTCCAACAGGCCACGACAGGGGTTGGGGACCCCT
GCTTTACAGGAAATAATGTGCTGATCCCTGGTTTAGATAATTGATTTTGAAATTTGATTCTATT
TAAAACTATTAGTTTTCCTGTAAGCTCTTCTTTAGCTGCATTTAACTGGTTTTAATATGATATA
CTGCAGTATCATTCGATTCAAACTATTTTCTAAGATTCGAAGATTTGCTGTAACTTATCCTTTT
ACCAATGGGTTATTTAGAAGTGTGCTGCTTAATTGCTAAGCTATTGGAAATTTGTCTACTTACC
TTTCTGTTCTTAATTGTCAGTTTAAATTCACTGTGATGTGGTCAGAGAATACAACCTAAATGTT
ATCAATCATTTGAAAAAATTTTAGGACTTAAATTAGGGGCCAGCATACAGTATACTTTGGTAAC
TGTTCCCTGTGTAAATAAAAAGAAAATCTGATCTAGAACTGTTAACTGTGGTATCTTACATAGT
CAACTTGATTAATTTAATGAATCATGTTGTTCAAATATTTTATATTCCTGTTGATGCTTTGACA
GCTGTTCTAATACTGAGAAGTGTGTGTGAAAATCTCCAACTATTATTATATGATTTTGATGTTT
CCTCATCTAATTCTGTTACACTTTGAAATATAAATTTTGAAGCTATTAGGTGCATATAGGTGAA
ATATTACATACAGAATATTTTATGATTTTTCTGTTTAATTGACAGTTTTACTATTACAAAATGT
TCTTGTGTTCTTCTAGTGATTTTTCTTGCTTTAAAGTCTATTAACTTGATATTATATAATAATC
AACTTTCTTATAGCTATTGTTTGCATGTTATAACGTCTATCCTTTTACTCTCCATTTCACTGTG
TTCTTATATTTAAACTGCCTTTCTTATCAATAGCACATACTTGAGTCTTGTTTTTGTATGCAGT
GTGACAATCTGTCTTTAATTGAAATGTTTAGTTATACATATACAATATAACAAATAAAATAGAT
GGATTTATATCTGCAGTCTTGCTATTTATTTTCTATTAACTCCATCTATTTTCATTTCATATTT
TTTCTCTTCTGCCTTCTTTTGAATAATCAGCTTTTTTGCTATTCTATTATTCTCCTCTATTAGC
TATTCTTGAATATTTTAATAATTAGCTAGAGATTCAATACAAACTCCTTATGTTCTACAGTCTA
CCTTAAGCTAGTTGTTTTTACCACCTCCTGAGGGTGCAAGAACTTTACAACACATGTAATCAAT
TCACTGTCCTCAGTCTTTGTGCTATTGCTGGCATATATTTTTACTTCTCCATGTTATATATTTC
ACAATACATCATAATTATTTTTAACAGTTATTTGTTTATATTTTTCAGATATATTTACCCAATC
CAGTGCTCTTTATTTCTCTTTCTTGCAGTTTTATGCTTTCATCTGGGATAACTTTCTTTCAGCC
TGAAGAGTCTATTTTAAAATTTTTTCTAGAAAAAATCTGCTGGCCATGACTATTTTCAACTTAT
TTTTGTCTAAAACATTTTTATTTTACATTTATTTATGAAGAATATTTTCTCTGAGTATAAAATG
TTAGGTTGGCTGCTTTTTTTATTTCAGGTTTCAGAGATGCCTTTCTATTTTCTCCTGGCACCTA
TAATTTCTGATAAAAAGTCAGCTGTAAGTTTTATTGTTGTTCCTTTAAAGGTTATGTGTCTCAT
TTCTCTGACCATATTTAGGGTCATCTCATTCTCTTTGGTTTTCAGCAGTCTGACTGTGGTGTGC
TTCAGTGGGGTTTTCTTTTACTCTACGTAAGATTCACCAGTCTGCAGTCTGTGAGTTGATAGCC
ATTCAACAAAAGAAAAGAACAGCAGCTACCATTGAAATCTCCACCTGATTAGACTTACAATAAT
CCACACTGTCATTCTCTAATATCCATCTGATTTTTTGCACAAGCCAAACAACCAAGTTAAGTGG
GAGAGGTAGTAGAAAGCAACACCCCTATGTCTTCCCAATTTTAGGTGGTGGCACTAAATCACTA
ATATTCTCCTTGAGGCATGGTATTGCTTTTGGTTTATTTTTTTCAAATAGAGGACTACGTTCAT
AGGTTTATATCACGAGAGTCCCTCATTCTATGGGTCAGTGGAACAACTGAGGATCTTGTCAGAT
AACAAGTATGTCTATTACAACTCACATTCAGGATCTGAGGAATTAACAGGATGGCTCATGGACC
TACTGGACCCATTGAGAAATGATGTCAACTGATGTTCACAAGCCCTCATCAGGCTTGAAGGACC
AGAGTGTCTTTATGGTCGCCAGGAATTATTACATGTTAGCTCAGTGACAGTGCCCCCAAATCCC
TGAAAGATCTGGATATTTCCTTTCCCTTGGTGAACAGTTATTCTGCTAAACGATCGCGTAGCTC
CTTCAGGAGGGCTAGAAGGAAAATATACAGCATGAACTTATTGGGTTATCCTCAGCTGTACCCA
GACTCACCTTCACTCACATGACTCTGTGTCTATGAACTGACTGAGGTTTGGGATTTGGCAAGGG
GCTGTGAATCTCTATTGAGGTGGCTTAAGTCAAGCTTTGGTTTACTCAGAACTAGAATTGTACT
TATCTAAAAGGACACAGTAAACTGATTACCTATTTCAGTCCTTGGGACTTCATGACCAATTCAC
CTGTCAAAGATCCCTGTAGGCTAAACTATTCAGATTGCTACTCTAGCTCTTTTGTGCAAGATTT
CACCTCCTAAATTAGACTGCACGTGGCCTGCTTAATGACTTGAACCACAATCTCTTAAATCAAA
GTCTTTCCTTCAAAAGCTACTTTAGTCCAGCTGACTACAAAAGATCATTTAACTGTTTGGTCAC
TGTGTGCCATGGACCACTGTCCCACTGCCAGCACTACTTAGATACTTACTGTTTTCAAATCCAT
TTCAGACACTAGTCCCAGATTTCTATTTCTTTAAGGATCTTTTGTCATCAATTCTGTGTCAATA
ATACTTGTTGGTTGGAGACAACAGAATCCATAGTAACTAGTCTAGGCAGGCATGTCAGATGATC
CTAGATGACCCTTATGCTCAAGTATTTGCCGGAAGGACTCACAGGACTCAGAAAAGCTCTTACA
CTCATGGTTATAGTTCATTACAGCAAATGGATACAGATTAAAATCAGCAAAGGGAAAAGGTACA
TGGGATGAAATCCAGGAGAAACTAGGTCCAGGCTTCCAGATATCTTCTCTGCTGGAGTCACATG
GAGATACACTTAATTGTCTCTGCAATAATGTGTAACACATATGAAGTAGCTTCCTGGTTGGCAA
GACTTCATAAATTTTGCCATTATTTGAAGATAACCCACACCTTGGTGTCCAGGGTTTTCACAGG
TATTAGTGGTCAGTCACATAGGCATATAGTACCTGTATGACTTCTATTCCAGCCACCGCAAACT
TCTCCTCTCCCTGCTGGCTCCTGAGCCAAAAACAGGCATTTACCATAAATCACTTTGTTAGGAT
GAACTTATCTGGCCAAACTGATACAGCATGACCCACAGCCTCAGGTATATAAAACACTCTCATC
AGGCAGAATGTTTCAGGGTCTCAGAGGTTAGTCAGGGGCTAGTCAAGGACCAATCCTGAAGGCA
GGCCTTTTTTTTTTTTTTTTTTTTTTTTTGAGACGGGGTCTTGCTCTGTCGCCCAGGCTGGAGT
GCAGTGGCATGATCTCAGCTCACTGCAAGCTCTGCCTCCCAGGTTCACGCCATTCTCCTGCCTC
AGCCTCCTGAGTAGCTGGGACTACAGGCACCCGCCACCATGCCCAGCTAATTTTTTGTATTTTT
AGTAGAGACTGGGTTTCACCGTATTAGCCAGGATGGTCTCGATCTCCTGACCTGGTGTTCCACC
TGCCTCAGCCTCCCAAAGTGCTGGGATTACAGGCGTGAGCCACCATGCCCAGCCAAGGCAGGCC
TTTTTTTGGAACCTACAGGGTTTGACCAACCCAGGACTGCTGAGTTAACCCTTTCCTGTGCAGC
AAGAAAAGATTTATTTAAAAAAAAAATGGAAAATCACAGTTTAATCAGAAGGGATGAAAAGACA
GCTTCTAGCTTGTATTTCTGAGAACATTTCCCAGAACCATATCATAAAACTGGCCTGCTAAGGA
AGATGTCTCTGCTAACCCAGAACAAGGTTAAATTGCATGTGGTTTATGGTTATATATGGGAATG
CCCTGGTTCTTATGGAAAAGATACAGGGATAAAGTGTTACAAAGTCTGTAACTTATGACAAATG
GGTCCATAGCTGGAGATAACAGATAAATGGATAGATAGAACAAATATGGCCAGATATAAACTAC
CAGGACATCTAGCTAAAGAAAATATGGTTATTGATTATACTATGCTCTAAACTTTTCTTTAGAT
TTAAAAAATGACCAAGAAATAAGGAAGTACAAAAAACTATGGCATGTTTCATATGAGAAGTGTT
TGACATAAAAGGGAAATTGGGAGGCTGGGTGTGGGGCTCACGCCTGTAATCTTAGCACTTTGGG
AGGCTGAAGTGGGAGGATCGCTTGAGCCCAGGAGTTCAAGACCAGTCTGGGTAACATAGTGAAA
CCTAATCTCTACTTAAATATATATATATAAAATAAATTGAAAAAAAGAGAGAAATAGGAAAAAA
GCTACTGAGAACTAGATAGTAGGTTTAGTAGGTTTGAAAGTCATGAAGAAGTTCAGACTGAAGG
ACTTCTAGATGTGAGGAGATTACAATCAGGTAATAAAATATAGTACAGTTGACACTTGGCACAC
ATAGAAATGCCACAGAAATATGGAAAAAAAGGAAAATATGATTGCCTTTGGGTGTTAACCTGTT
GGCTTATTCACATATGGACCAAAGCAGAGTTTGTTATAAGTCACTTAAAAAAAAGAGAAATGGC
TTCCAGGCCTGCAAGATATAATGGAAGCCTGACATTTCTAAAGAAAAGGGCTAGTCATCAATGA
AAACACTGGATAAAATCAGACTAGGAAAGTCTGGCTGTAACTTCCTAGGGGATATTTGCCCAAA
AGCTTCAACATACTCTCATTCCCGTCAATGATGCTTCACGGAAGTTGAATGTATCAGGTATCAA
TTGTCCAGTTCTTTCAGTTCCTGTTCTGTCACTTCCCTTTGTCTAGATTTCAAGCTTCCTAAGA
AGCTTCTGTTATGTGGGGACCCTGTGTGTCTTCTTACAGGGAAAAAATGCTCTGGTCACCTGCA
TACCAGTCCTTTCCAGCATTCCAGTCCCTCTGATGGGTACTTTTCAATTTAAATCAATAAACTG
GCATTCCGATTTTATAATTCCATCTCTTGAGTTTGTTAAATGTGAGTTTGATTGGTGGGCTGGC
TTAAATCAGTTTCTAAGCCTGCAATCATGAGGGCCAGACCTGAACTCTAGTCAACTCACCAAGT
TTGGCTCGTTTGTTCATTTGTGTGGCAAATGCGATTGGTGCCCTGGATCTTCCTACTGTATTGT
ATGCCAGTCCTAGTTTCAATTGCCAGCTCCTGCATTTCTTGCTTAGGGCTCTCTCTGACCAGCT
GGAGCTGCTGTGTCCCAACCACAGCAAGCCAGCCAGATGAGGATAACAACATTATACACTCACA
GGATAATTGTGAAGTGTGTGTTCTACACTGGCTCCCAGAGTTCTTCAACATAAGCAAATTCCAG
TTGCCCAAAGTGGACACTTGATTGATAATACACCCTTTTTGGTTTCCTCCCTTCCGTAACTCAC
TTACTTTCTTACCAGTGTTTCTAGGGATCACCTCCCAAATAAATTACTTGCACTGAAATCATTG
TCTCAAGGTCTGTTTTCGTGAGAAACACAAACTAAGACAACTTCGTCCATTCATTCATCATTTA
TTGAGCTTAATCAATTATTAATAAGTCCTTAAGGCACAAAATAAGTCCTTAGCAACATGTTATC
TCTTTTCTCATCACAGTATTATCCCCTTTACAAATGAAGAAATCAAGGTTGAAATAAATTAAGT
AGCTGACTCAGTATCACATAATCAGGACAGAGTGCTGGAAAGGACTGATGGCAGATGAAGATGG
CTGAGACTCCCAGATGCCCAGTCTTGAATGGAGAGTATAAAAAATGAGTTTTCTGCTGGGGTAA
TAATTTGAGTATATTATAAGATTGCTACGAAGCAATGAAAAAAGGGATTATTGAATGAGGAAAC
ATTTCAATGAAATATATTTTTGAAATTTTTGTTTTCTAGGAAAGCAACATGCTACAATAAGAGC
GCACACACATACACCTGTGGGTTTAAATCCTAGCACTACTAGTTTTTAGCTGTAAGACATGGGC
AAGTTGCTTACTTACTCTAAGCTCAATTTTCTAATCCATAAAGTATATAATATCCAAGAATGTT
GTATGAATTAATGAGATTTATTTAAACAACTGGCACCTAATGAGTGTACAAATTTAATTTGCAG
AAATGTACTGTCCTTTCAGGAACTTTGTGGTAAGTACTGAAATATGGATGCAATCAGTACTATG
CTTAGCAGTAACTTAAAAAAAGAAGCAATATAAAATTTGTATGTCATAAATGATAACTATATTA
CTATCTTTAAAAATCTTGGTCTTTCCATATGTAGTATGCTAATTTAATGTTACAAAATAAAAGT
AAAATCAATAATTTTAAAAAGTCAGACTTTAAGTATAAAATTTTGTTTGAAGCAATGCCAAAGA
ATCCACATAGTAACATTTATCATTTTGATAACAGAATTTTGTGTTCTGTATGTGGGCCTCAAGC
AGTACTAAAACAAAATAGTGTATCAATTATAAATGATACTCATGATTCTTCCCTAGTCAGATGT
TTTTTTCAGTTGAGAGATTCTGAAGAAAAACAATAAGTGTAGAATGTCTGTACCATCTTTAATA
CTAAGATTTAATTGAATCAATGCATTTCCTTAGATTTAAAAATCATTTTGTTAGAGTCTGTGTT
AGAATCAGTATGATACGTATTTAAAATGGAATTAGAGATATGGACTGTAGTAGGAATGACTTCA
AATAACTCAAATGATGCGCATGTCAGAAAGAAACAATTTCTAAGGGCTGCTTCATTGATTCAAG
TGGAACTGTTTTATTCAGAACAGCTCAGATTAGCAGCAGCAGCAGCTGGTGTTCAATGAAATGC
TTCCCTTTTCCACCTACTCTCCAGCTGGGTTAAGACAGGCACAAAGAGATTGGGATGACCAAGG
TCAAAGCATGATTTGATAGACTTCCAGGTTTTTGCTCCAGTAACTTCTTGTGTAGTCTTGGCTA
TAGAGGCAATTCGTGAAATTGGTGGTTGAAAAAGTCATAGCTTTAGAAGGACTTCATAGATCAT
CTTGTTCAATATCTTAATTTTATACTTCATATACCACGGTTCTAATTAAAATACTTTATTGTTG
CATATTTATTGGAATTGACAAGAAGTTAAGATTTCTTACATTTATTTCATTTGCCTGATTTTTC
TGTTCCCACTGAATTGTATAAGCAGGCACTACTGCACTTTGTATCTCTACTTGACATAGCTGAA
AAATGAAACATTCAAAGATACACTACTTTATTACATGTTAGCTCAGTAAAGGGGCCCTCTATTA
GACATGCTGGTCTCAGTTCTTAACTTTTACACTCACAAAGCCCGATTCACTCATTCAAACCTTT
TTTGAGGAGTTACCAGGTTTTAGCCATTGAGATAGATACTGGGAACACAAGAAGTCAAGGGTGC
TCTCCTCTCTCAAGAAGCTTAAGAGAAGACAGAAGTGCTAAGCACATACATCGAGTATGCGGAG
GGCACACAGAGGTATAAGCAATGACCCTCCTTGAGTAGATCTGAAAGGCTTCTCAGAGGAGGAG
CTTGAACTTGTCTAAAAGGTAGAATAAACTGCATGCAAAAGAGCTCCAAATGACTCTCAAGCTT
AAGTATTGCCTAAGCAATACATTCATGTGGGAAGTGGGAGATGGGGCTGGAATGGAGGCAGAGG
CCACATCCAATGGGCCGTTTTTGGAGTTTAGACATTATTCAGAAGGCCATGGTGAGCCACTGAT
AGAGTTAAGAAAGGCAGCAAGGTGATAACACATGTTTCTTAAGAAAACATACTGTGATGAGGAC
ATTTGGCAGGAAAGTAGTATAATATGAACCAGCATTCTCTGAATGTTGGGGTTAAAATAGTGAA
TGAGGTAATATGATCTTACCCTTCTGGAACTTACAGTCTAGTTCAGAAACACAACGGGTCTAAA
CACTAAGGGTCTAAATTAAGGTAGTAGCAGTGGAAATATACAGAAGGAAATGGATGTTATGATA
ATGATGATGATGATATTGCTTATTACCATTTATTGAACACCTCCCATGTGTCAGGCACTAAACA
CCTCATATGCACTATCTTTGTCCATTCTCCACACAGTAGCTAGAGTTATTCTATGGATCTTGTC
ATAACTCTCTCAAACCCTTCCAGAGGCTTCCCACCTCATTCAGCACAAATACCTAAGTTCTTAG
GAAAAGCTAAAAAGTCCTCCAGGCTGTGACCCACTGTGACTTCTCTGAGCTCATCTTCAACTCC
TCCTTTCTCTCTCTAGCCACAACGCCTTGCTACTGTTCCTAGATGACTCTAGGCACCTTTCTGC
TTCAGGGCCCCCTCTGTCTAGAAGGTTGTTTCCTTTGTATTTCCACAGTTCATTCCTCACTTCT
GTCAAGCCTCTGATTAAACGAATTCTTTTCAATAAGATCTTCCCTGGCCACTCGGCATGACGGA
ACTCCTGTAGCACTCCCTATTCCCTTTGCCTTGCTTTGTATTTCTCCTTAACATATCATATTCT
GACATAACACATATGTGTTGGTTTTCTTCCTCCATGGATCACAAACTCCATGAAGGTAGACTGT
TTTTGTTCATTGCTGTATTCTCAGCACTGATAACAGTGCTCGCACATATCCATTCAATAAATAT
GTACTGAATAGATGAATGAAGGATTACAATGGTCCTATGAGGATGGTACTATTATTTTTTCCCA
CTTTCCTAAAGAGGAAACAGACACCAATTGAGGTACTCAAGGTTGTATTTCTGGTAACTGGCAG
AACTGGGATTTGCGTGGGGCGGGGGGAAGCCTGCCTTTGTTGAGTTGTACTATGTGCCCAGCAT
TATTCTAAGAACTTTATGTACATTAACACTTAATCCCTACACCTCCATTTTATAGATGAGGAAA
CTAAATCAGAAAGGGGTTTACAATCTTGCCTGGTATCTGTGCACTTGCCAGTAAGTTGGGGGCC
TGTGCTCTCAGCTACTTTGCTACACTAATTCTAAAGCCAATTCTTTAGAGAGATACAACTTTGC
CTCTTAATTGCCAGGAGAGTGAGGGAAAGGAAGAAATCCAGGATTGCTACCATTATTATGACTG
TGGACCGCCATAGATGGTAGAGTCACCAATCAAGTCAAAGAATGTAAGATGAGTTGGTTTCATT
CTCCTCTAACTCTAGACTTCCACATCCAACTGCCTCCTGATATCTTTGCTTGGATATTAAGACA
CAAGGTCAGCTGCAGTTAAAGGGATTTAGAATAACCATGGCAGATCATAGAAGTTAGTAGCTGT
CTTACATCAGAGTTGACCTGGTACACAGGACCTGCCCAATGAAGTCCTCAGGGCCCCAACTTGT
TGCTCTGCAATCCCTAGAATGTTTCCCAGTGTCTTAGTCTTGTTTATGCTGCTGTAACAAAATA
CCTGAAACTGGGAAATTTGTGAAGAACAGAAATATACTTTTTCACAGTTCTAAAGGCTGGGAAG
TACAAGCTCAAGACCCCCAGCAGGTTTAGTGTCTGGTGAGGGCTGCAGTTTCCAAGGTGGGACC
TTACTGCTATATCCTCTGGAGGGGGTGAATGCTGTGTCCTCGTGTGGCTAAAGGGATGGAAGGG
CAAAAGAAGGCCTAAGCTAGTTCCCTCCAGTCCTTTTACAAGGTATTAATCCATCCATGAGGCA
GGGCCCTCATGATTTCATCAGCTGTTAGTATCATCACAATGGGGATTAAGTTTCAACATGAATT
TTGAAGGGGACACCATCATTGAAACCACAGCATCTTTCAACATGGTTTACTACCCCAACTATTT
AATACATACATTCATCAGAAAAGGACAAAAGAAAGGGCAAGTGACACCCAATTTCTTTTAGAAA
ATGATCTGGAAGTTGCATACATAATTTCTGCTCACATCCTACTGGCCAGAACTTAAGAGATATG
GCCACACTTAAGGGTGTATTATAGGCAACCATGTGTCTGGCTAGATGTTCTATTATCATGGATA
AAGAGCAGAATGCATAAGAGTCAGCTAGGAAGCAGTTTGTACAACAGATATCCAACAGGTATCT
CAAATTTAACATTACCAAAACTAAATTCCTGCTTGTTCTCCCAAAACCTGCTCCTTTGCACTGT
TATCACATCTCAGTCCATGGTGGAAGACAGAACAAACTCAGGGGTTTGATGTTAATCATGTTGA
GCTTGAGGTGCCTGTAGAACACCCAGTACAGGTTTCCAATAAGGGGTTGAAGATACTGTGTCTG
ATGCTTCTGACTGAGATCTGTGTTCAAGATTCAGACTTGATTCCTGACTGAAATAAGTGATAGA
AGAGTATACGTAAATGGTACTGTCAAAGATTATAACATGGTAGGCAGAGAACCAAGAATGGGGA
GGGAGGATTCTGCACAGGAGACCTGAGAAAGAGTGGCCTAAGACAAGGCAGAAGAGCAGAGAAT
GTAAGGTGACAGAACCAAGGGAGCAGTTTCCAGGAGGCAGTGGTCAGCAGTGTGGAAGGCTGCA
GAGAGATCAGATGTTATGAAAACTGAGAAGCAACCTTTCAATTTGGCCATTATGCCACAGTGAA
ATTGATACAAGCAATGTATACAGTAAAATGGAGGGAGCTGCTAAATTTCTGTAGTTGAAGAGGA
ATTTGGAAGGTAATAGCAATCTAAGTATAGACAGCTTTTCTAAATAGAAGGGACAAAGTAGAGG
GAGAATTTATGAACAGGAGTGGGAATATTTAAGGGAGTAAGAGTCTTGAGTCAATCTCTCTTCC
CTCAAGAAATAAAAATTACCTGTAGAAAGGACAGAAGGATGGCTTCTACCTTAAGACTCGAGGT
CAGAAAGAATGCAAATTTAGACAGGTCTGCAGGCATAAGGGTGGGGGTATGTAGGAGTTCATGC
CTGATGTGTGCAGTACTAAACAATGATACATATACTACTACTACTACCACTACTAAGCACTAAC
ATTTACTGAGCACTTATCATATGCCAGTGTTCTTCATGGAGCTAAGTTTGCTTAACAGTATTTC
TTAAATAATGGGAGCTATTCATATATTGAAAGCTTAGTGTTTTTCAGAAGTTGAGTGTGTAACA
TTTGGATGTGGCCTAAAAATAGCAACCGTGACTTCTATCCCACCATATCTGAATTTTGTCTCTT
CCATGCTCGACTTATTTCTGATCCTGGGGCATGCTGAGTAGTAAAAAAAGTTGTTCTTGCATTT
TGATTCATCTGTTATGACTGGCATCAATAAGAAATCTTCAGCAATAAAACAGCTCTGTCAAAAA
ACAACTTCACCAGCAATTTAGGGCAAAATGTTTCCTTTAAAAAAAAGTTTGTTGGCTGGGCATG
GTGGATCATGCCTGTAATCCCAGCACTTTGGGAGGCCGAGGCGGGCGGATCACGAGGTCAGGAG
ATCGAGCCCATCCTGGCTAACACGGTGAAACCCCGTCTCTACTAAAAATACAAAAAATTAGCCA
GGCGTGGTGGCGGGTGCCTGTAGTCCCAGCTACTTGGGAGGCTGAGGCAGGAGAATGGCATGAA
CCTGGGAGGTGGAGCTTGCAATGAGCCAAGATTGCGCCACTGCACTCCAGCATGGGCGACGGAG
CAAGACTCTGTCTCAAAAAAATAATAATAATAATAAAATAAAAAGTTTGTTAGTATTAGCAGAT
ACATATTACTAGGTACCCCCCATGCTCAATGAAGTGTTGGGTTACTCTGAAAAAGTGTCCAATC
TTACAGGTGTGACTTCCTCTGGAACTGCAAGTTCTTGAGGGCAGGGATAGAGATTGTTCATCTT
TTATTGTTTCCCAAGGTCTGGCACATAAATAGTAGTTCAAGTAGTCCAACATATGACACATAAA
TAGTTCTGGGTAAACATTAGCTCAAAGTTTGCTCAAGGCATTGATGCAGATGCTTATAAAATTA
AATTGCTCTTTTTACTGTATACCTGAAATGAGAAAATTATTAGAAACTACTGGGTCTCATTTGG
ACTAGAAAAGTCTGAAGAAATCACTGTTCACTTAGTAGATACAAAGTTGTTTTTCTGGGAAAAA
CACACCTAAAAAAAAAAAAACACATGGGTCTGATATGGGAGAATTTTTTTATTTTTCATATAAA
CAGGTGTATAGGGAAGTGGAACACTGCTAGTATCTTAAGGATTCCTGAAAGTATTTATGTTGGT
AAGTTTGATGAGCAAACATTTCTAAATTTGAAAATGGATAAAACAAAAAGCACAGTGAAATCTT
GAAAGAATTCTAATACTAATGTCACACATAAGAATACAGATTTAAAATTAATGAACTAAAACGG
ATATACGTAATTTCTCCAGGAGGAATTAAGATCCTCCTTGATCTCAGTAAAGCAATGAATAGTC
ATTCTGACATCTAGCTAAAAGCAGACTTATTTCCCTTCGGTCTTTAATCTTTTTGGAACTCAGG
GATTTCCGCCACACTTTGATACACCAGGCCATTATAAAATTACACTTAATTTATTGTCAATTTC
TACTTAAAAGGAAGAATACCTATAAGTATCTCCATATGCTAGATATAGTAGAAATGGTATGCTT
TAATTAGTCCAACAATAAAGTCCTTAAAGATTTACATTCTTTGTTGCCTATTTTGAAAACTTGC
GAAGATAAATTTTTTTAAAGATTATAATTCTTACTAGTCCTTTCCATGAAATATAACAGCAAAT
AGTTTCCAAATGTGGATGTGTAATGAGAGGAAATGAAAAGTGAGTAAAGAGCTGAGTTAAAACA
TCTTAAAATCGAATATGGCTTAATACTATCATTATCATCAGTTGTCCTGGAATAACTTAAATTC
TTCTAGCTTATTGTTGGTTTTTCAATGGTTGGTTATAGTTGTGAGGCGGATACACACACACACC
CACACACTTGGCTATACAAAAAAGATGTTCCGAGACTGACAGTTAAAAATTACACTGCTGGCCG
AGCACTGTGGCTCATGCCTGTAGTCCCAAAACTTTGGGAGGCCGAGGTGGGAGGATCACTTGAG
GCCAGGAGCTCGAGACCAGCCTGGGCCTAACATAGCAAGACACCATTTCTAAGTTTAAAAATAA
AAATAAATTTAAAAAAGATACACTGCTATGTACGACTGTTGATATAAAAAATCTGAATACTAGA
CATGGGTACTCATCTAGTACTTCAAGGGCTTATCAACAAAGTTGCAAGTTGTAACACTATGAAT
TGTTAGTGATACTCTTTTGGCTTTGCTAGCAAGTGTTGTAAAGCTATACACACACACACACACA
CACACACACATACACACACACACCTTTTAAAATGGTGACCCTGGTACCAAATATGACTTTAAAT
GGATTTAATTTTAATGGCTTTAACTACGTTCAGCTGTCATATGGATCAAAATTAGCCTCTATCC
AGCTGGGGTCAACCAGGGAGCCACTTTTCTTAACCGACGACCTACTGAACGTCAACAACTGCAG
GAGACGGGACTTTACCTTCGTCTCTGGTAAACTAGTTGACACATCCTGTGTTGGCAAGAGGCCT
AAGTAGATGACCTTGGTCCTCTAAAATCTGGCCTGCACTCTCGGGGCACCCCTGCAACATCTAC
AAAGGCAGCTCCAGATAGAAAAGGGTTGGGGTCGAAAAGCCAATAACGGCAGGCACCTGCCCCG
CCTCGGGGCTGGGGGGCTATTCCAGCGGCTTCAGCTAACTTTCAGAGCCATTCGTTTCCCAACA
AAGTCTGAGGCGTTCCTCTGCTGGGTACACCAAGGGGCTCTGCAACCCTCCTGGGGGGGGGGGT
GCCCAGAGGGCTTCCGGAAGTCCCAGGTTTATTCTTTCGGGTCACAGACAGCAGAAACTAAAAA
GAGGGATTACCCTTTCTGTCCAGTCGCAAGATGGCGACCGAGCCTGGTGGGACTCCGAGGGGCC
GCAGGCCACCTCCTCTTCCCAATGGCCCGTGCGCCGGCGGCGACGGCAAGCGGGAGGGAGGCGG
GGCCGGCGAAGGAAGGAGGGGCGGAGCGCGGCGCCCTCCCGCGCGTCTTGGCCCCGCCCCACGT
CCCCGCGTCCCGGCCTGGAGCCCTCGCCCGGCCGGGCGGCGCGCGCTGCCTGCCGGGATACTCG
GCCCGCCCAGCCAGTCCTCCCGTCTTGCGCCGCGGCCGCGAGATCCGTGTGTCTCCCAAGATGG
TGGCGCTGGGCTCGGGGTGACTACAGGAGACGACGGGGCCTTTTCCCTTCGCCAGGACCCGACA
CACCAGGCTTCGCTCGCTCGCGCACCCCTCCGCCGCGTAGCCATCCGCCAGCGCGGGCGCCCGC
CATCCGCCGCCTACTTACGCTTCACCTCTGCCGACCCGGCGCGCTCGGCTGCGGGCGGCGGCGC
CTCCTTCGGCTCCTCCTCGGAATAGCTCGCGGCCTGTAGCCCCTGGCAGGAGGGCCCCTCAGCC
CCCCGGTGTGGACAGGCAGCGGCGGCTGGCGACGAACGCCGGGATTTCGGCGGCCCCGGCGCTC
CCTTTCCCGGCCTCGTTTTCCGGATAAGGAAGCGCGGGTCCCGCATGAGCCCCGGCGGTGGCGG
CAGCGAAAGAGAACGAGGCGGTGGCGGGCGGAGGCGGCGGGCGAGGGCGACTACGACCAGTGAG
GCGGCCGCCGCAGCCCAGGCGCGGGGGCGACGACAGGTCAGTGTTGCCGCGGCCTGCGCCAGGC
GGCGCTGGCTCCCCTCCGTCACTCGGCCGGCCTTCGGGGCCCGCTGTGGCGAGGTCGACACCCC
CCTTCCCCGCCCCCCGCCGCCGAGGCGAGTGTTTGGGGGCGCGTGGTCCGAAGGGGCTGGTGCC
AGAAGTAGGCCCCTGGTGGCCGCGGCTGCTGCAGCCGTAACTGTCAGTCCTGGCTGAGCGACGG
CGGGAGGGTTTTGTCGCCCGAGGGGACGCGAGCGGGCCCGGGGCGGGGGGGGACGTGCGAGGCG
TCGAGATTTGGGCCTCCTAGGAGCCAGGCTCTTCGAGCCAGCCGGGGCCCCAGACAGGGAAGGG
CAGGCCCTTTCCTTCAAAGGGGAGCCCTTTCTCGGCGTTTTCAAGGTTTTTGGCTCTCTTGGGG
AAGACATATTTAGCCGTGTGCTTGGTGGGTTGGGGTTTTGGGGGTGGATTGATGGGAAGGGAGG
GCGGATGAAGTGGTATGTCAAGCCCAAGGGTTGTGCGCACAGGTTACTCTGTGTTACCGGCCAC
CAGGATTTCTGAAGTTGAACGTGAGTTATTGGCTTTGCCAGAGACTGCTGTGTTATATGCAGAC
CTGTATGCAAGCAGTTGGCCTTTTTTCCCCCCCCTTTTCAGTGTAGAAAATGAAAAGGATGCTT
TCCTCATCTTGGTGGTAAAGGCTTTTGTTGGTAAAGGTAGAATTGAATGTACCAAATGCCTTAG
TCCGTAAAATTTTAGAAATAATTTTAATACAGACACTGGTGAAGCTTGGCAACCTTGAAAGAGA
ATTTAGCGTCTACATTTTTTAAATGACTTTTTATGGATATGCTAAATTAGTAACAGTCCAAAAT
CTGTTTGAGATTATTAAGTGGCGAGGGTGCTGTTGAAAATGTAAACTAATAGCATATGGGGTTT
ACAGTGCACAGTTAACCTCAATCATGAAGAAATGTGGATATGACCCGTAATTTTGGATCATTTT
ACTGCCTGCAATATTGAGAGAAGCAGCAAATTATTACAGTTTTTTTTGGGAGACGACCTAAAGT
TTAAGAAATACAACTGTTGAAAGTTACCTGTCAGAGACACAAAGGTACCCAATCAATCTTGTTG
AATAAATTGGACAAGTGGGATAAGGTGTTTGTCTCACACTTCTGATCAATAAGTACTCTTACTT
AAGAAGTGATTTGGTAAATCATGTAAAATTTAGAATTTAGGAGAGATAAGAAAGTTGTAACTTG
GTGTGTATAGTGGAAATAGCTTTGAAATTAGATCCTGTTTTTAAATCCAAGCTACTTACCACAT
TTTTAGTGAAATGAATACATTATTAATGATGCTACTGATAAGCCCTAAGGATGACCAGAAGCCC
TTTTAAGAAACACTAATACAGTTGACCAAAAAATAAAAAAGGAAATAGCTTCAAGAAATAATTT
TCAATCTTTGTGTATTTATAATATACACAGGAGAAATAAAAGAACAGAATATGAAGAGGTAACT
TATAATCTTTACAAAATTAGAATGTAAACTATGCTAGTCTTACTGTGTCAATATTTATTAACCT
GCATTAGTTTGCTAAGTAGTATGCTCACTATCCTCTGGACATTGCCCCTGTCCCTGAGGAGCTT
AACTTTATACAGAGATACAAAATACTGTGTTTTGATTTCCTTTCAGAGGATTTATAAGCTACTT
ATGTTTTATCTGTCCTAAATTCTGCCCTTTTTTTTCCCATGGCTGAAAAAATAACTTCCTAAGA
AAGGTACATACTAGTCAGAGGTGTGGGGAAAACGTGTTCTCATCCCACTTGTGCAGTCCATTTA
ACAGGATTTATTGGGTACCTACAGTGTCTTTGACATGTAACATTTAATCAGTTGTATTTCTTAA
ACTTTAGATAATTTCTATTTCGATCATTTCTATTTCAATCTCCTCCCTCTCCCCTCCAAAAAAA
TTTACTGTGTAAACTGTGATAATACACTGTTTCTCTCAGTGTCAGTCAGTGGAATGATCCAGAT
TTATAGGTCATAGCAAAATTTTTCATCCCAAGCTCTTGAAATGATGTTTCCAAATGTCCATTTT
CTTAAATGACTGCTAGTTATGTTCTCCAATCATGTTACACATATTTTGTTTAGACTTATAATTG
TGTAGGTCTAAATGTAGAAACATTACATTATGTACATATGCAGCTGCTGAAAGAAAATTAGTTT
TCTAATTTTTAGATCGGGGTGAAGACAAACTTTTAGAAGTGATCTTTCGGTAAAATACTGGATG
AAGTCTTAGGTGCTCTTTTTATGTTGGTTCAGGACAAAAGTTTTTTGACTGCTTTTTGAGTTTT
CCCCTCACTGCCAAATGTAAGGATAAAATGTAGTAACAAGACATCTTGGTATGTATAGTGGTAA
TATCTTTGGAATTAGACCCTAATTTTAATTCTGGCCTTATTAATCTTGGTAAGCCACAGTTTTG
TCATCTGTAAAATGGGTTGGTGAAACTTCCCCAGAGTTGTGAGGCTTAAAGTATGTAAAACGCC
TAACACGTAGGCACTCAATAAGTATTACCTTAGAAATCTTCCTATCCAGTTTTTCTGAATGGTC
TGGAAGCACCTTGTATGTAGTGGACTACAGCATGTTATCCTTTATGTTGTTTATTTCTTTTCTC
ATTAGCTATGAACTTTTTAAGGCCAGGAATTCCTAGTGTTTACCAGAGAATGTGTTACTAGATG
TCTTAGTCTGTTCTGGCTGCTATTACAAAACACCACAGACAGCATGGCTTATAAACAACAGAAG
TTTATTTCTCACGGTTCTGGGGGCTGAGAAGTCCAAGATCAAGGTACCAACAAATTTGACATCT
TAGTGAGGGCCCTCTTCCCTTGTTCATAGATAGCTAGCTGTCTTTTCACCATTACCTCATGTAG
TAGAAGAGTAGACGGGGCTCTATAGGGCCTCTTTTATAGAGGCATTAATTCCATTAATGATGGC
AAAGCTTCCCAAAGGCCCCACCTCTTAATACTATCACCTTGGAGGTTAGGCTTTCAACATGAAT
TTTGGGGGACACATTCAGACCACAGCACTAGAGCAGGCATGAATTGGTTATTGGTAAATCAGTA
AATGATCAACTAACATTTATTAGGTTCCTCCTGTGGCTTCTATGAGCTGAGCTAAGTACAGGAC
CCACAAAGACAAGAAAGATGTCTGTCTTTTTTGAATGCTTATAGTTGAGTGACTTTCTCACATT
TGTGTGTGTGTGTGTGTGTGTGTGTGTGTAAAACCCTATCACATAATGGAACTTCATTTTCCCA
TGCCCTTCCAATCTAAGAGCAGTTCTATGGACTCAATACCTCCTACCCACCAGCTGCTACTGTA
AAGATTTACTTTTTTACTCCTTTTAAAAAGTTCAACCCTCTGGCACATGCATTACTTCATTCCT
TGGTCCCAGCCCACCACCCCAGTATCTGGCACGTAGTGTATTAGTATTTGTGTGTGTGTGTGTG
TGTGTGTGTGTGTGTGTGTGTGTGTGTGTGTGTGTATGTATATAGAATTTAAAATTTTTACTCC
GGCAGTTGTCTTCAAATACTCTAATTTTGGGGGGCATGATTATCATTCTGTCTTCTGGCAGTAC
ATGGATAGATGCTACCTGAGTAGGATTGTCTATGAGGTCAGCTGCTCCAGTCATTTTATACAAG
AGGAAATTGGAGTGCAAAGCTTTAAAATGATTTTCTTAAAGTCACCCAGGTGTCAGAACCAGAA
CCAGAGTCCATGTCTCTAGTTCAGGACTCTGCACACATTGGCACATGTTCTGTCCTACCTTTTG
GGATGAATAGTTATACTTGCTTTGTGATTACTCACCCACCTTTTTGGAGACATATGAGTGTTTT
GTACACTATTAATAGAAGGCAGCCTTCACAAAATGCCATCTGCAGATCTGTTATTTCTTCTAAT
AAAGGAAATGATGTAAAATTACCTTAAATAGAAATAGTGTTAATTCTTCTGGAGACATATTCCA
TAATCCAAACACGAAATGTTTGAAATACTTGAAGATGCCATATAAATAAGTCCTTTGTAAAAAG
ATAATATTAAAAATTTTTGTTTTATAGAGATGGGGGTCTCACTGTGTTGCCCAGGCTGGTCTTG
AACTCCTCAGCTTTTACTTTAGCTTCCCAGTGTGTTGGGATTACAGGCATGAGCCACAATACCT
GGCCAAGTCCTTTTTTTTAATCAAATGACTTATTAATACACAGTTTCTTTGCCAGCTTTTGTTT
TCATTTGCTATCAAAAATGTTGCTTAGTAGTGCTTTGATCTGAGTTATCAATAACAGGTAAATG
CCATTATGGATAATAATTCAAAAAGAAGCTTATTAATTATTAGGCCTATCTGAGAGTGAAGTAA
AGTTAGCATTTTCTTTTTGTTTATTTTACTTATTGTTTATTTGTTTAGAGACAGGGTCTCGCTG
TGTTGCCCAAGTTGGAGTGCAGTGGTGCTGTCATAACTCATTGCAGTCTCAGGCTGGAGTGATC
CTCCCATCTCACCCTCCTGAGTAGGTGGGATTAGCATATGCCACCATGCCTGGCTAATTTTTTT
ATTTTTTAATTTTTTTGTGGAGATGGGGTCTTGCCGTGTTCAGGTTGGTTTCAAACTCCTGGTC
TCAAGCGGCTTGGCCTCCCAAGGTGCTAGGATTACAGGTGTGAGCTACCATGCCCAGCTGAGCA
TTTTTAAAAAATACTGGTCTTTGTACATGAGTCGTTACTATTTGATTCTAAGCCTTATGACTGA
TATCCCTAAAAATTATTTATAAAATTTTAAGTGCATCAGAGTCATTGAAATGGAATGAGCACTG
TCTTTTGGTCTTGAGGTTGTTTTAACTAGCTTCGTAATGGTCATGAGCAGGTTATTTAGCTTTT
GAAGCCTTCGTTCCTTCTTTTGTCAAATGAAAGTGATAGTTGCTTTGTTTTAAAAGAGTATGCT
TTTCAAACGTGATCATTCTTGAAAATGTAGATTAAGAGCTTTTTAGAGGCCAAGTGCCATGGCT
CACGCCTGTCATCCCAGCACTTTGGGAAGCCGAGGCGGGCGGATCACGAGATCAGGAGATCGAG
ACCATCCTGGCCAACACGGTGAAACCTCATCTCTACTAAAATACAAAAAAAAAATTAGCCGGAC
GTGGTGGCGCATGCCTGTAGTCCCAACTACTCAGGAGGCTGAGGCAGGGGAAACGCTTGAACAC
GGGAGGCGGAGGTTGCAGTGAGCCAAGATCATGCTACTGCCTGGCGACAGAGTGAGACTGCCTC
TCAAAGAAAAAAAAAAAAACTTTTTAGAAACGTTGTTTTGGGGAATTTTTAGTGTGTGAATTCA
TTTTAAATATGTGAACTCCATTTGACATGGACAATGGATCTTAATAGTTTGTGATACATTATTC
AGTGTATTAATCAAAAGTCAAATCTCTTTTGTAATTTGCTGTACCAGACCTGTTGTGTTAATCT
GATCAATGGCAAGTGAATCATAAGATTGACCTGAAAGGAAAAGATATTTTTAGGGGACATAGAA
TCATATTGGATAAGATTATTCCTATAGTTCATTTTCAGGAAATCTGAACAGGAATCTCGTGTAA
GGAATCACATGTGAATTATAGAAGAGAGTCTGATGTAAGACTTTATTTATTAAGGTCTGCATCT
GTGTTTCAAGTAAGAGCATAGAGGTAGTATATGAGCCCTGAACACTCATACAAGTAGGATAAAA
TTTTTCTTAAAACTGTTGGCTGGACAGTGTAGAGTCTAATGCTGTGACTCTTAACAGTTTTGTG
TGTTCCAGAAGTTCATCTCCTCAGGATCAATTGAGGTTAGTCTAGCACAGACTTTAGTTATAGA
AACCAAACATGAGAAAAATTTTTTGACCCATGTCTTCGTAGCATGCAGGCTTGGTAGCTCTTTC
CTAGTTCTGGATGTGTGCCCGTTGTATATACTTTCAGCAATTCAGGATGTTCCTTTTCATATTT
AAGACAAGCACTCTTGCATACAAATTACATAGCTGTGGGCTGAATGAAGATGCTACCATTGCTG
TACTTCTACACAGGTTCTTTTCATGTAATGAAATCAGAAGGGATGGCTTTTCCATTTCAAGCTT
CTGTTAATAAGTAGTCTTAGAATTGCCTTTGTGTACTGTAGCCAACACCCCAACTCATATATTT
TTATAATCTTTGTGATGCAGAATAAATTCTGAAACTTTCCACCTCCTAATTAAATATAGAAATA
CTGAAAACAATAAAGCACCATGTATTCACTATGGACATATGTCACATTTTGTCACTTTCTTGCA
CTCTTGTAATCCCTCTCTAAAGCTCATGTTAAACTCATGTTCTTTATTTTCTTCTCAGATGATT
CCTTTGGCACTGCATGTAACAGTACAGGAATTTCTTCCTTTCGTACTTCCAACAAATACTTTTT
TGTCTCTTAACAGATGTGAACCATAATGCTGTTCTTTAGAAAGCAAAGGATGAAGTGCACAGCA
GTGTTTTCTATATAAAGCTAATGGGTCATAAGGGGAAATATGGGTTATTTCCAGCTTGTTAATT
GTCTTAATACATTTTTCCTAAGAATTCTATTGTGCCTCTGTGGTTATATGCACCAATATTGATA
ATCTTACAGCGTGCCATAAATAAGGATTTTTATATTATCTTTGCTAAATATTTTAAGTTTTTGT
TTTGGGAATTGAAAAAATCTTTTGTGCATCTATGGTATGTGATACGTATACTAATGGATAAATC
CTGTTTCACACAAAGATTAATTGGGTAACATTTTTCTTTCCTAGTTTTCTGAAGTCAGTGGTCA
TGTTGTGTGTTGGTGATTGAAAATATGCTGTTTAGGCTGGGCGCCTTGGCTCACTCCTGTAATC
CCAGCACTTTGGGAGGCCGAGGCACACGGATCACCTGAGGTCAGGAGTTCGAGACCAGCCTGGC
CAACATGGTGAAACCCTGTCTCTACTAAAAATACAAAAATTAGCTAGGCGTGGTGGCACACGTC
TGTAATCCCAGTTACTTGGGAGGCTGAGGCACGAGAATCGCTTGAACCTGGGAGGCAGAGGTTG
CAGTGAGCTGAGATCACACCATTGCACTCCAGCCTGGGCAAGCAAGATTTCATCTCAAAAAAGA
AAAGAAAAGAAAAGAAAATATGCTGTTTAAGATCATTCAAATTCAGTTTCTAAGAAGTGTAAAG
TTTTCCTAATTACTTAAGCTGCTTCATTCTCATTCCCTTTTGTAACACATTTTGCCATCTCTGT
AACATGTTGAACATGTTTAAAGAACAGAGTGGGAGAATAGGAAGTTACGTGGATAAAGTTTGGT
AATCAAGGTAGGAATACATCCTGCTTCACAGAATGGGAATATTTCTGAAAAGTTGACTGTAGTA
TTGAAGAACACTTAAGTGTAGTGTATTAGACGAACTGAGCTAATTTTGGTCTTTTATACACATA
AATCAAGATTTCTGCATTTCAAATCATGCATATTTCTTTAAATTTATTGCTCAGTGTACTGTAT
TTCAAATATTGTAAAAATGAAAAGATCTTGTGGTCTTATTTAACTCAGTATGGGATAAGTCCTT
CCCACCTTCCCATTCTTATTCCCTCTTTTAGGCTGAATGAAAGGCTGGGGGAGTGAATTTAAAA
ATAGATGGATCTCAGCTGGAGAATGGGTTGGTCACGTATCTCAACTGATATTAAATAATTTCTT
AAATCCTGGATTGAAGATTATAAGAAAACAGTAAATGTTTGCTCTCAGTTCATTTCTTTGTTTT
GTTTTTTCATGTACAGTTTTAGGCACTTGTAACTGCTGGATGCATGAACTACTTTCTAGTTCTT
TCAGGCCTACTTTTAGATATCTGCTGTGGGGACAGTCCTCAACACTTAAAGTAATTTTAAATAG
TGATAGGCAGAACTAGGGGTCAGTTTTACTGCCTATGCCTGTGCTGAATTTGACATGGCATTCC
TAGGAGGGGAGGTGGAACGAGGGCGGTGCACACATTGTCCATATTGCTAGTCAGTGCCAGATTT
TGAGAAACTGGGGCTAATCTCTCTCTTAAATGGTCAAGATCTAATTCCCCAAAGTGCTGTTTCA
GATAACCTAGTATTTGTCCAAAATTGCCCTGCTACAATAGTATTTATCTTATATATATGTATTA
TATATATAATATATATATAATCTTATATATATGTATTATATATATTATATATATAATCTTATAT
ATGTATTATATATATAATATATATATAATCTTATATATATGTATTATATATATAATATATATAT
AATCTTATATATATGTATTATATATATAATATATATATAATCTTATATATATGTATTATATATA
TAATATATATATAATCTTATATATATGTATTATATATATAATATATATATAATCTTATATATAT
GTATTATATATATATAATACATATAAATATATATATATATATATATATAGGTTTTTTTTTGTTT
TTCCTCCTGAGATGTAGTCTCGCCCTGTTGCCCAGGCTGGTGTGCAATGGTGCAGTCTTAGCTC
ACTGCAGCCTCCGCCTCCTGGGTTCAAGTGATTCTCCTGCCTCAGCCTCCTGAGTAGCAGAGAC
TACAGGTGTGCACTACCACGCCCGGCTAATTTTTGTATTTTTAGTAGATACAGGGTTTCACCAT
GTTGGCCATGCTGGTTTCAAACTCCTAACCTCAGGTGATCTGCCTGCCTCAACCTCTGAAAGCA
CTGGGATTACAGGCATGAGCCACCACACCAATCCTATTACATTCTTAAAAAAATGTTTGTTTGT
TTGTTTTTGTAGAGATGAAGTTTCACTATATTACCCAGCCTGGTCTTGACTCCTGGGCTCAAGT
GATCCTCCTGCCTTGGCGTCCCAAAGTGCTGGGATTATAGACATGAGCCACTGCGTCCAGCCTG
TATCATATTTTTATTCTGATATCTTTTGTAATTTTGTTTTTAGTGAAGTAGGGAGGTTTATATT
AGATAATCTTCCAAGGTAATTATCAGCTTGGACATTATATGCTAATATGTCTTTTGTTTGTTTT
TAAAATAAGTGTATATGTTTTAATTGTAAAATTTTTAAAACATCCAGAAAATGACATAGCAAGT
GCCTATTTATCCACCACCTGGAAATATAAATTATGTCCATTTTTATCCCTTGGGGGAAAGGATG
CAGTTATATAATCACTTTTAACAACCCCAACTGTTCAAGTCCTGTTACCCGTCTCTACACAAAG
TAACAACTGTCTTGAGATTCTTAAGTAAATTTCACATCCTTGTTTTATACTTTTACTATACATG
TGTACATGTATCTGTAAGTGATATGTGGTGTTGGTTTTTGTATTAAATTTTTACAGACATGTAT
AACGCTGTCTTTCAATAGCTTGATTTTTTTAAACTCAGTATCAAGTTTTTGAGGTGTATATGTT
GATGCGTGTACATGTAGTTTATTCAGTTTCACTGCTGTATGGCACTTCATTTTATGCATAGATA
AAATGTATGTGTCCCACTATTGATGGATATTTTGAGTTGTTTTCTTTTTGCTATTATAAATAAT
GCTGCAGTGTTTATTTTTACATGAGTGTATGTGCAGGTGTTTTGGGAAAAACCGTAGAAGTGAA
ATTGCTGGTTACTAGATATTGGTAAATTCTTTTCCTAAATGGATATGCCCATGTATACTTTATC
AGCATTATGTGGGCATTTCTAGCATCCTCACCCATACTTGGGATTGTCAGATTTTAAAATAGTT
ACTAATTTGAGGCATGTGAAATGCTATCTCATTTTAATTTACAGTTGCTTGATTATTCTGAATC
GTAGGTAAAAGGATAGGAAATTTATTATTTTCTGTATGTTTTAAATATCTCAAAAATAAATGTT
TTTAAAGGAAAGGCACACTATCGTACAACATTTATGTTGAAAAGGACCATGAAGATCATTTCAT
CCAACCTTCTTACTTTACAGTGCAGCCAAAGAAAGTTACCAGAATAAGAGTAAAATATAAAATC
CTGCCCATTGTAGTATTCTCTGGTAGTATGAGCCTTAATTTTTGATGTAAAAATCATGTCTTTT
TCTTTATGATTGTGCTTTTTGTTTAAGAAATCCTCTTTTTATATTGTCTGCTAATAGTTGAGGA
TTTTTTGTTACTCTTTGTTATTTGCATTTAGGTTCATCTGAAAGGAGTTAGGATTTTTTATTTT
AAATATGAAAAGCTAGTTGTTCCAGCATTAAGTATTGAATTTTTAAAATTTTGTATTGAGGTTA
AATATACATACATAATTTACCATCTTTTTTAAGTGTACAGTTCATTTTTAATGATTTTTTAAGT
GTACAGTTTTTTAAGTGTACAGTTCAGCAATAATAAATACAGTTATGTTCTTCTGCCTTTGTCC
TCTCACTCCCCACTTCGGCTGCTAGTAACCACCAGTCTATGCTTATCTTCTTGAGATATACTTT
TTTAGCCCATGTATGAGTGAGAACATGTGGTATTTGTCTTTCTGTGTTTGGCTTATTTCACTTA
ACATAATGGCTTCTTGTTCCATCCGTGTTCCTGCAAATTACAGGGTTTTATTCTTATTTTTATA
GCTGAGTAGTATTCCACTGTGTGTATAAACTACATTTTAAAGTCTATTCGTCCTAATTATTGAA
TTCTCCCCACCCCACCCACCTTTTTTTTTTTTTTTTTTTTTCCAGGAGACAGGGTCTCCCTCTC
TTCCCCAGGCTGGAGTGCAGTGCCATGATCATAGCTCCCTGCATCCTTGAACCCCCTGGCCTCA
AGCGATAGTCTTGCCTCAGCCTCCCAAGTAGCTGGGACTGCAGGCAGGCACCTCCAGGCCCTGC
TAAGTTTTGTGGGTTTTTTTTTTTTTTTTTTTTTTTTTTTTTGTAGAGATGGGGTCTTCCTATG
TTGCCCACTTTGGTTTGTTAGTTTTAGTAGTTTATAATTCCTTTGTATTATTGTGAACAAATAA
CAGTTATTTTGAAATAGAAGAGTGCTTTCTATTTAAAATTTTGCTTTGATAGGGTTCTGTCTAC
TTTCTGAGCCTTACCTTCCTGTGCATTCCCCTTTGGTCAGTACCATCTTACCACACAGGCTGCT
TTTAGTTTCTTGAGTTATCCATCTTCCTTCTATCTTATAGGGCTCAACCTAAATATTACTCAGG
GAAGCCTTCCTTGAGAGTCTTTCTTTTTGCACTCATTGGTTTGGGGCTTCTTTATATTATACTG
TTAATTGAATTCTGAGTCGTGTTTATCTCAACTGAGATTAGGTAATTTATTTTGAGAACAGTAA
ATGTTTATCATCTGTCATTCTTTTTTTCATGCCCACATTCAGTTATTTGTAAGTGCTGAATGTA
TAAGTGTTCATCTACAGATAAGGATCAGTTCTTTTTTCCCCAACTAGAAATGTTACTCCAGTTG
ATAATAACAGCTCAAACGGAAATATAAGGCAGAGGTGAGAGAGTCACTTGAGGCCACGAATTGG
AGACCAGCCTGGGCAACAAAGTGAGACCTTATCCCTATGAAAAATAAAATTAGCCCAGCTTGGG
AGGCTGAGGTGGGGGGATTGCTTGAACCCTGGAGTTCCAGGCTGCAGTGAGTTGTTATCACACC
ACTGCACTCCAGCCGCGGTGACAGACCCTGTCCCTTAAAAAAAAAAAAATTATAACCAGCCACA
TTGAAATATTTTGTAAGACATTAATAGAAGACTAAATGTTCTCATATGTTTACTGTTTGTGTTA
AGTACGTATGGTTTTTAGAACAACAGTTTTCTATCAAGTGTGGTCTGGTTCACTTAGAATTAGA
TGGGGTAGATTTTTTGAAAAGCGTTTTGAAGTTAAATTAGAAGCTAATGGTTTTTTGTCACTAA
ATCAAATTTTGTTTTATGCGTTTAAGTGCTACATTTATTTGATAGGCAGGTGAATGTAACAGAT
TAAACTTGAAAATAATGTAGCTATGATTCCTTCAAGGAAAAACCCACCTACCTTTAGTTCTTTT
AGTATATTGCATTATAAGTCTTTTGTGTGTGCATATATAGCAGGCATAAATTGGCATAATTCTG
GGTTTAATTTTAGGCTAGGCGCTATGGAAGTTTGAACTCTAATTACAGTGTAAAAATAACCCAG
AGGAAAATTTCAGAATAATAAATCATTGGGACTATATGGTACAGATGTCCCTGTTTTCAGATTG
TAGAATTGTTGACCAGTAGGAATGCTTTATGATTAAAAGCCAGGAAATTAAAGTAATTCTGATG
ATTTGGTATTTCTAAAGATTTTCATAGATGACAGTTGAGAACTACTCTCAGATGAGATTCTTCC
ATTGCTGGTAAGTCATGTAATAAAACACTTAAGTAAATATTTTAATGAGGAATGATAACATTTT
AATATTGGCACATTGTGAGTCTACCTTCTGTAGTTAGTAGGTAAATAAGAAATAAACATACCTT
ATTTAAAAAAAAAATTTCAAAGAACCCAAGGGAGGGGAGAGCCAAGGTGCACATTAGAAGGAGA
TTACAGTACTCTTCCAGTGCACAGTGGATGTGTTGTGATTCCAGAGTTCTATGTATGTATGACT
GTGTAGCCCAGATTTACCAGCATGGGCTCAGTTTTAAATGTTAGGTTAGGCTCATAAATCATGA
AAAGTTCCTAGGAATTCTAACATTTTTACATTTCCAGAAGAGAGCCTCTTGTATTTTGTGGTAG
TTGCCCCACCATCCCTCAAATATTGGTGTGTAGAATCTATTTTTGAGGATGAGGAAATACAGAA
AAGAAAAATAAAAAGCTATTTTTTTTTAAGTATAAGAAGAAATGAAAGGTTTCTCTTCGCTTCA
TAGCCAATTCTAAAACTTAACAGTTGTTAAGTGTGTTTAACTGTAGAGGGAAAAAAATGCCTGG
AAGGAAATTACCAATCAAATATAAAATGTAAGATAGCTGTAAAAGTACTGATATACATACAGCT
TTTTTCCTTTTGGGGAGGCAGTATTTAAAAAAAATCCTACCAAACATATTCTCTAACTTTCAAA
ACTGTTAATAGCTTTATTGAGATAATTCACATACTATAAAATTCACCCTTTTAAAGTTTTTAGT
ATATTCACAGTTCTGCAGCCATCACCACTATTGAATTTTAGAACATTTTCATCATCCCCAAAAT
AAATCCTAACCTGTTAGCAGCCATTCCCCATTCTTTCCTGGCCTCTGGAAACCACTAATCTGCT
TTGTCTCTATGAATTTATTTATTCTGGATATTTCATATAAATGGGAATCATACAGTATGTGTCC
TTTTGTGTGTGGGTTCTTTTACTTACCATGGTATTTTCAAGGTATGTTCATTGACATGTATGTC
TGTTTTTTTTGGGTAACACACACTCTTGATTACTGTAGCTTTGTAGTAAGTCTTGAAACCAGGA
AATGTGAGTATTCTAACTTATTCTTTTTCAAAATTGTTTTGGGTCTCTGGGTTTCTCAAATTTC
CATAGAATTTTAGGGTTAGCTTGCAAAAAAATGCAACTGGGATTTTAATAGGACTTGGATTTGT
GTTGAATCTGTAGGTCTATTTGGGAAGTATTGCTACCTTAATACTGTCTTCTCATTCATGAGCA
TGGGATATCTTTCCATTTATTAAGGTGTTCTTTAATTTCTTTCATGTTTTGTAGTTTTCAGTAT
ACAGGTCTTGTACTTATTACTTTAAAAATTTATTTCTAAGAATTTTATGTTTTGATGGTATTGC
AAATTGAATTTTTTAAAATTTCATTTCAGGTTTTTAATTCCTTGTGCATAGATTTTTGTGTACT
AAAACTTGTGTTTCATATAATTTTTTTTTCCTCTTAACTGTGTTATAGGAATCTTTCAAATGGT
TGGCTACATACACATCCATCTGATTCACTTAGTAAACATTTCTTGAACACCTACTGTGTAGTGG
GCCTTTTTTTCTAGGTCCTGGTAGAGACAGCAGTGTATAAAACCCACTCAGTCCTTTGCTTTCA
TGGCACTTAAATCCTAAAAAGAGATCATAAAGATAGGTAAAATATATGTTACATAGTGCTAAGG
AGAAGAAACAAATAAGGGAAGTGTTTGCAGGTGAAAAGGATGATGGGAAATTTTAGATAGGGTA
GGTAGAGAAAACTTCACTGAAATGGTAACTTGGGTGAAGAACTGAGGGAGATGAGAGAGCTAGC
CATGTGGATATTTGGGGGAAGAGCATTTCAGGCAGAGTGTAGTGAATAGGGAAACCCCAAAGTG
AGAGTGCCTGATGTGTTTGAAGAACTCGAGGTGTGGCCAGTGTAGCTTTAGAAAAGTAATTGAT
GGGAAATAGAGAGAATGGAGGGTACAGATATTTTGATCATAATAAGGTGTTTGATTTTTACCTA
TAAGTGAGATGGGAAGCTATTGGAAGTCTTTGAGCAGAGTAGTGAAAAAGTATGACTTATGTTT
AGCAAGGTCACTCTGGTTGCTGTGTTAATCATAGACTGAAAAAGGGGACAAGGGCAGAAACAGA
CCAATTGGTAGACCATTAGTACAGATTAAGACGTGACTGAAATGGACAGAGTGGTCACAGTGAG
GGTGGTGAGAAGTGGTTGAATTCTTGATATATGTTGAAATAGAGTCCATAGGATTTGGTAGTAG
ACCAGATGAAAGTGGGAAATAAAGGGAGGAGTAAAACATGATGCCAAAGTTTTTTGACCAGAGC
AGCTGAGAGAATGGAGTTGGATAATTCTTATAAATTATTGCATTAAATCTGTGCATCATATCAC
ATGCCCACTTAACTACAGGTAATTTTACCATATAAGCTTGTGGGGGGAAAAGAGGAAAAACAGA
AAAAAGGATGGAAAAGCATGTTTTAAAATATAATAAATTAGGCCTTAATGAAAAATTGTCCTTG
AGAAACCTGAGATGGAATACTCTAAATCTGATGTGTTCTCTCAATCTTTAGAACTGAACTAGGA
ATTAACTCTGAGAGTTCATGATGTTATGAGACCCTTTGCCTGTTTTGGAAATTCGGTTGTTTTC
AATATTTGCTTTTGCAATGATGGTACCTGTTATTTTTTCTGTAAGGTAGATTCCTACAAATAAG
ATGTCAGGCCATTAGATCATGTCAATCTAAATATTTGATAGGTTTTGCCAATTACCTTTTAAAA
AATAATTTTAGCAAGTTTCATTCCTTAAAATATATTAGGGACTGCTCATTTACCTTGTTTTGAT
TCATGTATATATAAAATCTGTTTAAGTCTTTATAAATAACCTGGGAAATTGCCTGGTTTTTTGG
TCCATTTCTATTGTGGTGTTTGTTTTTTAAATTTGTAAGCATTCTTTATATATCATTTAATTCT
TTATTATAATTTGCAAATATTATCTCTACCTACCTTTAATTTTATAGTATCTAGTCATGTGAAA
TTTTAAAGTTTTTATGTAGGCAAATCTGTCCATCTTTTATGGTTTCTGGGTTTCAGCATGCTAA
CAAAGTTGATATAAATATGATCTCATATATATATTACAAAAACTTTTTAGAGATAGGATCTCAC
TCTGTCACCCGGGCTGGAGCAACCATAGCTCCCTGCAACCTCAAACTCCTGGGCTCAAGTGATC
CTCCTGCCTCAGCTTCCCAAGTAGCTAAGATTATAGACATGTGCCACTATAGCTAGTTGGTTTT
TTTTTGGTTGGTTGGTTTTTTTTTTTTTTTTTGGTAGAGAACGTATCTCAGTGTGTTGCCCAGA
CTGGTCTCAAACTTCTGGCCTCAAGCTATCCTCCCACCTCAGCCTCCCAAAGTGCTGGGATTAC
AGGCATGAGCTACTATACCTGGCCAATCTCTTGTATTTTCTTCTGATACTTTTGTGATTTTACT
TTATAGACTTTAATTTAGAAAGTTAGATATGAGCATTCAGCATATTTTTGGATATGGTATGATA
TGGTTTAGATCTGGATTGCATTTGTCATGGTTTGGGAATTATATCTACCTTTTATTTTCTTCCA
CTTGGGTAGCTGATTGTCTTGTAAGGCATCCTTTCCCCACTGATAATGCCACCTTTTTCTATAT
TAAGTTCTCACAAACTTTGAAAAAAATCAAGTTTATTGATGTATAATTGATACACAATGAAATG
CACCCATTTTAACTATATAGTTTTATTGATTTTGATAAATATATACACTATCACTGCTACTACA
ATCAAGCTTTAGAACAATTGAATTACCTCCAAAAAGTTCCTTCCTGTCTGTAGTCATTTCTCCC
TCTTGGCCCCAGGCAACCACTGATCTGTTTATTAGTTTTCCTACTCTAGAATTGCATATCCATG
GAATCAAGTAACATGTACTCTTCCATGCCTGGTTTCTTTCACTTGGCATGTTTTTGAGATTCAT
CCAATTTGTGTTTGTCACAGATTTTTGTTTGTTTCGTTTTTTTAAATACAATATGACTATACCA
CAGTTCATTCATTTTCTGGTGAACATTTAAATTGTTTTCAGTTTTAGGCTGTTGCGAATAAAGG
TACTGTTTTGTGTGCATGTTTTTGTGTGAACATATGTTTTCATTTTTCTAGGGTAAATACCTTA
AATTGGAATTACTGGGTCACATGGTAAGTTTATGTTTAAATGTATAAGGAACTGCCAAACTATT
TTCCAGATGATTATACTACTTTACATTCCCATCATCAGTGTATGAGAGTTCCAGTTCTCCAAAT
GTCAGCATTTGATGTTCTCAGTCTTTAATTGCACACGTTCTGAGAGGTGTGTAGTGGTATCCCA
TTGCAGTGTTTTTTTTTAAGTGGTACTAAAATACACAACATCAAATATACCATTTTAACTATTT
TTAAGTGTACAGTTCAGTGACATAAAGTACATTCACATTATTCTGCAGCCATCACCACTATCCA
TCTCTGAAACTTTTTCATCTTCTCAGTCTGAAACCCTATCCATTAAACAGTAACTCTTCATTGC
CTACTCTCCCTATCTGCTGGTAATCACTGTTCTACTTCATATGTGAATTTGACTCTTCCAGGTA
CTTCATATAAGTGGAATCATACGATTTTTGTCCTTTTGTGTCTGGCACATTTCATTTAATAATA
ATAATGTCCTTGCGTTAGGCCATTCTTGCGTTGCCTTAAATGAATATCTGAGACTGGGTAATTT
AAAAAGAAAAGAAGTTTATTTGGCCCCTGGTGCTGCAGGCTGTACAAGCATGGCGCCACCATCT
GCTCAGCTTCTGGGGAGCCCTCATGGTGAGTTTACTCATGGTGGAAGGCAAAGGGGGAGCAGGC
GTGTCATATGGCGAGAGCAGGAGCAAGAGAGAGGGTGGGGAAGTGGCACACCCTTTTTAAACGA
CCACATCTGCGAACTCACTCTGTATCCCAAGGAGAGCACCAAGCTATGAGGGATTGCTCCCATG
ACCCAGTTACCTCCCACCAGGCCCCACCCCCAACATTGGGGATTATATTTTAACATGAGATTTG
GGCAGGACAAATATTCAAACCGTATCAGTCATCAAAGTTCATTCATGTTGTAGCATGTATCCAA
ATTTGATTCTTTAAGGTTGAGTAATATTTCATTGTGTGCATATATCACTTTTTGTTTTATTTAT
TTATTTGTCAGTGGACATTTGGACTCTTTGTAACTTTTAACTATGTGAATGATGCTGCTATGAA
CATTGGTATATAAGTATCTGTTTGAGTCCCTTCTTTTAATTCTTTTGGTTATGTACTTAGGAGT
GGAATTGCTGGGTCATGTGGTAATTGTATGTTTAATTTTTTTAGAGACTGCCATACTGTTTTCC
ACAGTGACTGCACCATTTTACATTCCCACCAGCAGTGAATAAGGGTTCCAATTTCTCTGCATCT
TTTCTAACACTTGGTAATTTCTGTTTTTTATTTAAAATAATTGCCATTCTAATGGGTGTAAAAA
GTCATTATGGTCTTAATTTACATTTTCTTCATGACTGCTGTTGAGAAGTTTTCTTTTGTGTGTC
TGTTCAAATATTTTCACATTCTTTTCAGAACATATGTTCTTTGTCAGATAGATAAATGTATTTC
TTCTGTATCCTTACTGATTTTCTGTCCACTTACTAATTACCGAGAGAGGAATGTTGAAATCTTC
AACTATAATTTTGAGTTTATCTTCCTTCTGCTCTGTTCATTTTTTCTTCATATGCTTTGAAGCA
AAGTCTTTAAAGTTGTGTCCTTAGGTGTGTTCACATTTAGGACTGTTAGATCTCCTTGATTAAT
TGATGTCTTTATCATTATGAAATGTCTCTGTTTATCGCTGGTAATGTTCCTTATCTAAAGTCTA
CTTTGATATTAGTGTATCCATTCCTTTCTTATGATTAATTTTTGCACATTATATCATTTTGCTG
TGTTTTTATTTTCAGCTTATTGGTCATTTAATGTCTTTCTTTTAGATAGCATATAGTTGCATCT
TGCTTTCAGATTGACTTTTTACGATCGTTGCTTTTTGATTAAGTCCATTTACATTTATTGCAGT
TGGCAATATAATTGTGTTTAAGACACTATTTGTTTTCTATTTGTCTAATCTTTTCTTCCTTTTC
TGCCTTCTTTTCGGTTAATGGAGCATTTTTGGAATTTTGTATTGTCTCCATTATTAGCTTGTCC
CTCTGTTTTTTTTCCCCCCAGTGATACTTCTAGGATTTACAATATGCACCTTTAACTTACCTCA
ATCTACCTTCAAAAATATTATTCCTCTTAATGTATAATGTAAGAATTTTGGAATAGTATACATC
CATTTACTCTCCCTCATTCTTTTTGGTATTATGATCTTAACATTTTACTTCTATATGTGTTATA
GATTCCACAGCAGCGTTTGAAACTATTTCTTTCAAAGGCCAGTTGTCTTACGAAGAAACTTAAA
AACTAAGAATAACATTCCTTTATATTTACCTTCATATTTACCATTTTTATTTATTTTTCTTTGT
GGAGATTCCTTTTTGTAGATCCAAGTTTGTTGCTGGTGTTATTTTCCTTTTACTTGAAAAAATT
TCTTAAACATTTCTAAAGTATAGGTCTACTGGCTACAAAATCTTTCAGCTTTTGTTTGTATGGA
AATGTCTTTATTTCACTCTTCATTTTATCGTACAATTTTTCAAACCACACAGACTTTAAAATAA
TTGTAGAATATAATTTCACAACATTTTGTTCTATTTGTTATTGTCTTCTATCTTGTCCATCTAT
CCATCCTGTCTTCTTCTAGTGTCTTTTTTTATGCTTTTCAAAGTAAGTTGCAGTTGTTAGTATA
CCACACCCTTAACTTGAGCATGTATGTTAACTAAAACTTAGTAGTTTAGTATTTTTAACTAGAA
TTTACTATGCTGTTCCAGTTATTTATTGCTGTGTAATACATTGCCCCAAACTTAGTGCCTTAAA
TATCAGCAACTATTTATTTTGCTCATGAATCTCTCATTTGGTCAGGGATTGGCAAGGAGAGCTT
GTCTTTGACAATACATTGTAACAACTGTTTACATAGCATTACATTGTATTTGGTATTATAAGTA
ATCTAGAGATGGTTTAACATATTTGAAGATGCATGTAGGTTATATGCAAATACCATGCCATTTT
ATATAAGGGATTCGAAGCATCCTTGGATTTTGGGATCCATGGGAGTCCTGGAACCAGTCCTCTG
TAGATAGTTGAGGTATGACTGTAGTCTTTGGATTGAAAGACTCACTAATTTCACTCAGATTCAA
GGAGAAGGGCCATAGACTCCACCACCTGATGAAAAAATGACAGTGTTACATTGTTAAGAGCATG
TGGAATGGCTTCTATTGTGACCATCTTTGGAAAATACAGTTGACCACATATACCATTTCATGAG
CTGTGACAAATGTATACACCTTTGCCAGAAAGTTCTATCAGGGTATAGAACCTACACCTCTTCC
TAGTTATTCCTTCCCCCCACACCACCCCTAGAACCTAACATTGTTCAGATTGTTTTCATCTTAG
TTTAGCTTGCGCAGAACTTTATGTAAGTAGAGTCATACAATACTTATATGTTGTAAAAAGTTTC
TTTTTGCTCAACATAATGTTTTTGAGATTCATCCATATTGTTGAATGTTTCGGTGGTTTGTGAC
TTCATCTGTTAAGGGACACCTGGGCTGTTTTCAGTTCCAGGCTGTTTTCAGTTCCCAGCTATTA
TAAAAAGGTACAATGAACATTTGTGTAAAAAAAGAAGTCTCTGTGTGGACATATATGTTTCTTT
TTTCTTGGTTAGTTGATGTGTTTTTCTTTCAGCATTTGTTTTTGATGAGAAGTCTGGTTATACA
GTCATGTGCCGCATGCTGACATTTCAGTCAACGAGATCATAGTTGACAATAGTCATTTAAGATT
ATACCACCATATTTTCACTGTACCTTTTCTAGGTTTAGATGTATAAATACTTACACTTTGTTAC
AGTTGCTTACAGTATTCATTCAGTATAGTAACATGCTGTACAGCTTTATAACCTAGGAGCACCC
GGGCTATACCATGTATCCTAGTTCTGTAGTAAGCTATACCATCTAATCTAGATTTGTGTAAGTA
CGTTCTATGATATTTGCACAATGATTACCATGCTGAGAGTTGTTGGGGTTTTGTTTTTTTTTTC
TTTTTGAATCTGTACATTTATGGTTTTCCTTAAATTTGGAAGATTGTTGCCATTGTTTCTTCTA
TATATATATTTTTTACCACCTTTACTCCTCTTTCATCATTTCCTCTTTTTCTGGGACTCCAGTT
ACACATATGTTAGACTGTTTGGTATTATCCACAGGTCATTAACATCATATTCATTATTTTTCAG
TCTTTTTATTTCCCTGTGCTTCAGTTTGCTTTGTCTTCTATTTTACTGTTTTTTCTCCTCCCTT
GTTAAATAATTTCAGATATTTCGCTTTATATTTCAAGAAATTCCATTTGATTCATTTTATTCAT
GTATTTTTTTGAATTCTTGAGCATATTTGTAATAGCTGTTTGAAAATCAGGTTACTTATTGATT
TCTTGAATTTTTTCACTCTGAATTTGTTGGTTGGTTTTTTCTCTTGGTCATGGGTTGTATTTTC
CTAGTATTTATTTTATTTTATTTTTTGTTTTTTACATATTTAATGATTTTTTAAGATTGGATGC
TGGACATTGTAATATTTTGTTGTTGGGTGTCTAGATTTTGTTTTCTTTTAGAGTGTTTTGTTTT
GTAGCCACCTAAATAACTTGCTGGTCAGTTTTATCCCTTCAGGGCTACATTGGAGTTTGTCTAG
AGAAGGCTTTGCTTTAATGGTAGTAGCTCCTACCTAAGGCATGATCCTTCTGAGTCTTTACTAA
ATGCTCCCAGTGTCACCGTGTTCTCTTCACTCAGGTTAGAGAGAATTTTAGGAGTCTCCCAGTC
CTTTATAAGTACTGGGAATATTTCAGCTTACTGCCCCTCTTTCACTAGCTTCATGGAGTCACTT
ACCTTAATCGTGTACAGTTAGTACTCAGCAGTGGACTCTCAAATGGCAGATTTCTGAAGCCATT
TCTCTGCCTTCTCAGACAAGCACTTGTCTGGCACTTGTCATCAATTTTACCCACTTCAGCTTTC
TCAGCTCTGATCTCTTGATTCTGAACTCAGTAAAACTGCTGTCTTCTCACTGGATTTCCCCTCC
ATTGACTGCAGGTTGGAAATTACCTCCAGGCAGACATCCAAGCAGTGGATGGCTCACCTCCTTT
GTTTTTGTCCTTTCATGCATTATAGTCCTCTGCTGCCTGTTATCCAAGGTCTGAAAATTCTCGT
TTGAGAAGGAGCAACTGTCAGATACTTTCGGATCTTTCTCTGAAATTAGCTTTCTGTTGTGCTT
GTTGTGCTTACCTCTTTGTCTAATGCTAATACCATATGAGGTTTTTTTTTTTTTTTTTAGAATG
TTTTAAGCTATACACTTCTAAGTGTATAGCTTGTTGCACTTTCACACACTGAGCACCCGTTTAG
CTAGCATTCAGATCAAAAAACAGCAGCAGCTCCTGCGTAGCCCCTTGTGCTCTGTGTCACTACC
CCTTTTCTTCCATGTGTAACCACTGTCCTGACTTCTCATAGCATAGATTAGATTTGCCTGTTTT
CATCCTTTAACTAGAATCAAGCACATTTAGTTGTGTCTGACTTCTTTTAATGTATTTGTGAGAT
TTATTCACATTGTTGGTTGTAGTAATTGTTTATGATAGTCGCTGTATATTCCATTTTAAGAATG
GAAGTGATATTCCATTTTAAGAATATGCCATAGTTTAATCTGCTGTTACGGACATTTAATCATT
TTTGGTTTGGAACTGTTAAGAATATTGCTGCTGCTCTAAACATTTCAGTGATTGTCTTCTTGTG
AATATGTTTACTTCTGTTTGGTATACTTAAGAGTGAAATTTTTAAGTCATAAGGTATGCATATA
TTTAGTGTATACTCAGTTTTTTAAAGGCATGCCGATTTTAATTATTCCATTTAAGTTAATATGT
TTTAATATCTGGTAGAGAAAGGAACATTATTAGAAACAATTGTTTTGGTTATTCTTACATATTT
AGTCTTTGAAAAAACGTTTCTTAAATTGGCATTTACTTATATTTGTGTTAATTTGGGAAGAAAT
GGTATCTTTGTTCATCTATGAGTCACATTTGGAACTAGCTCAAAGGCCATGGGTCCTGATTCCT
TTTTTTTTTTTTTTTTTTTCTTGTAGAGGGGAGGTATGGTGTCAAGAAAACATAGTCACCATTA
TTACGAAAAGTAAAATATGGAAGAGATGATCCCTACCATCAATCAGCTTACAACTAGAGGCACT
GACAAATGTATACAGATAGCTGTAATGTAAGGTATAACATAATAAATATAAGGAGAAATTAATA
AATTATTTTATGGTATTAAGAATGTATAATAAGACATGAAGGAGATGGTGATTATGTATAAGAG
TTTAGATAACCCTGTGAGATACAGTGTTTAGAATGAGGCCAGTTTTTAACATGGCATGATAGGT
GGGTGGGGGATAGAGGTTAGTAATGGATAGAGGACCATTTCCGAGTGAAGTCTCCTCAGAAGCA
AAAGCATTGAAGTCAGAAACACAAATATCCAGTACATTATAGCCAATAGTCAAATGATGGGACT
AGATCTGGTAACTGGTGATGATGTCATATAGTGGGCAGTAGTGCTGGGATGGCTGGGGTCAGGG
CCACATGGCAGGGGCCTCAAATAAAATGTGAAGGAGTTAGAAATTTATTCTGTTGGCATGAGAA
AGTGTTAAAGCTTTTGAATATAGAATGATATGTGTAACTTTGAGTTTTTAGGAAGATAATTCTA
GTGGCAAAGTTGAGGATAAAGGCAAAAGGGAAACCAGATGAAATAATGGGGCACTGTAGTAGGA
CAGTGTTGGTAAGTTTGAGGAAAAAAGGAGTAAGAGATATTTTTAAGGAAATTCATAGGGTTTG
AGTATAAATTAGAAGGCAAGTACAAGGGAATAAGCGTGGTCTTTAGAATCAGACAAATTAGGGT
TTGAATCTTAACCTTGCATTTACTAAAGGACTGAAAGCTAGTTCCCTAAGAGAAAAATTTTCTC
TTTTGTAAGATGCTTATTCTATAAACATTTGCAAGCACTGGAAGTATAAAAGCAAATGATACAC
ATGTTCTCTTGAAGTCTGGTCTAGTCTGTAAGAAGAGGCTGCACTGGCAGGTTTTGACCAAATT
GTGTAGAATCTCATCTGACGTGATTGGGAGCTTTGGCTTTATTCTGGAAGCAACTAAAAGCAAT
CAAAAAAGACTGGGGAATAATGTGAGAATGTAGAAATGTAGGGGAATTTCTAAGGATGAATTGG
TAGCAATTGGTAAACAATTTGATATACTGGGAAAGAGAGAGACTCTTTAGAATATGACTGAGAA
TCTTCCAATTTGAGCAGCTGGACTGATAGTGCTACCATCATTACCTCACAGGGTTTTTCTTAGT
GTGTCTTGTAAATCTTGCTTGTCCCTCATAAAGAATGGTTTTCCTTTTCTTCAGACTAAGGACT
TTTTATTACAGTACACAACTTAACACCCCCTGTATCTCTCCATCACAACACTTAACTGTGCATT
GTTATTGCATAATTAATTGTGGTTTTTATTTGTGCCCATTTCCTTCACCACATGAGGGCAAGAA
ATTATGTCTAGTTTTTCTACCTCTGCATCTTCATCCACCTAGTAGGCACTCAAAATCTAGTAGA
CAGTTTTTGAATTAATGATTATGAAGAATTTGAAGGCTGAACAAGTATCAACCTACACAGTGGT
TAGGGCAAATAAAAATGAGGGGGAGAAAGTAATGAATGGCACCAATTTTTTTAGTCCTTAACCA
GGACAGTGAGATTAGGCAGAGTGAGTTCTTTGGGAAAATAATTGAGTTTGATATTAGATACGTT
GAATGTAGATGTTAATGAGACATCCAAATGGATGTGTACCGTTGGTAATATGTAGTTGCAGGCA
TACCATGCCAGACATAAGGACTTAAAGATACAGAGTTAGGGGTTTGTAAGCATAGAGGTGCTAT
GGGAATGGATGACATTGCGCAGAAAATTGGTTTAGGTTAAGAAAAGTGGCTATTGACAGAAACT
TGGAGAGAGGCCATGTATGAGGTAGGAAGATGAACTAGCAGAAGACCTTATTGCCTTTAGAGAG
ATGGAAGAAAATGAGTGGATACAACATTAGGGAAGCCAGAGGAGGTGAGGAGTCATTAGCAGTC
TTAAAAGCTACAAAAATTAAGTAGGATTAAGGGGCCAAAGCACTGGGAATATACAGAAATGATG
GAAACTTTACAAGAAAAATTTCAGTAGATAGAAAAATAGCAGCTGAGCTAGTGGTTTTCACGTC
TTTTTGGCAGTAAAAGGAAAGAAGATAAAGAAATGGTACTCTGAGGAGCTAATGGAGTTGAGGG
AATTATGTTGCAAGTATTATTTGAAAGCAGTATTTAAAACTTTTTAAGTCTTAACTGCAGAATG
GACTGGAATAAGGAGAAACTAAAGCAGGAGGACTTAGGAAAATTGTTGTAGTAGTCCTTTAATT
GTGAAATGTGTAAACGGACAAAACTTAGACAGTTTTAATAAGTAGCATTATCCATAAATATAAG
ACACCATAAACATGATGCAAAGGAGAACAGAAGTGTTGGGTGTTATAATAAAAAGTGATAGAAA
AAAACAGTTTGTGAAATTAAGTTTTGAAACAGCTTTAGAAGGAGGTGAAGGAAAAATAGAAATG
AATAAAATGAAAATTTTAGGGAGGTAGATATAGATCAACATAGGCATTTATTATTAGACTATAC
TTATTTATAAGATTATTAGACTGAGCCCTCTGTTGAAGAGATAAAGTAAGAGAACTATGTCTGG
TATTAGGATTCTTATGGTTAAATTGGCAAGTGAGTGTGCCTAATATTTGTAGTGCTTACAGTTT
TTGTTATTCTGTATTTTTATAGATTTGGTATATTTCCTCCATGATTATGTGTTCCTAACTCATA
ATTTGGAAGGCAGGGCTTCTTAAAATGAGATCTGTGTGTCATGTGAAGTATATGTTAAAATGCA
AATTCATTTCTGCGTTCTTGGGTTGGTCTGAAAAAAATTAAAAAAAAAATTCATTTACCCCTAC
TTCATGCACTGAATGAAAATCTTTGCGAGTAGAGCCCAAGTAGTTGCATTTTTAGATTTCCCCA
GGTAATTCTATTATAGACTGAAGTTTGAAAGTCAGTCTCTAAAACCTTAAAAGTTGGTTAATTT
TATTTCAAAATGTTTTATATGCAGTGTATTCACTTTCTACCATCAACCTCTAGGACACTGGATA
GGTTTCTTAACCTTTGAGATTTGCTTTCTTTATATGTAATGCAGTACATGAAAGGACTTTATTT
TTGTTCCATTGAGGAATGTTCTGATTGTATACAAACAAGTTAAGAATTTACAGTGATAATTTAG
GGGCTTGCTTTAAGCTTCAGGATTTTTTTTTAAGAGTTAAGTTTTGATATATATTAAGTGGTTG
ATTTAATAAATTATAATTATCGTCGTATATTGAAGTCCTGTTTTGTGCCAGGTAGGCATTATAC
TATAACTTTTTAACGCATTTTCTCTGCTACAATAACCTGACATTGGAGACTCAAGGGTCAGAGG
GTCGGCCCAAGGCTGCTACTCTAGAAAGTGGTAGATGCCCAGATTTGTACCTAACATAGCCGAT
GCTTAGCATATAGAATTTAATTAAAAACAAAATTGCTATAGGTATTTGAAAGGATTAGAGGGAT
ATAGTCAAATAGTTTTCATATGTGTTTAGCCCTTTTGGAGGGAGGATAGAGTAAATAAATAGAA
GGCACTAAGCTTTATGGATGAATATGGGAAATATGGCTTTGCCTGCTGAAAGCACTCAGGCCAC
AGGGCCTGCCTACCTATGCTTTCCTCATCCCGGCAACAGCAGTAATTTGTTTCTTGATACGTTT
TTGGATGTTCATAATTATAGAAAATAAATTTGCCTTTATGATGAAATTTAGTACCTTTTTTTTT
GAAAGTTCATCCAGTTGTTTATGTAAAAAGAATTGAGTGAGGTGTTTTATATTTTAGGTTAAAA
ATCTGTAAGAGCCTGATTTTAGAATTCACCAGCTCCTCAGAAGTTTGGCGAAATATGGTATGTT
TTTGTTTTATTTTTTGGAATTCGTATTGCATGTGTATCTTAATGTCATAATAATGACAGATGTG
TTCAGCAAATTTGGGCCCCTGCCATTTTTAAAGCATTGTGTCAGGTGCTCACTAACTACACTTC
CAAGACTGTATGAGGGACCCAGGTAGGGTGCTGAAAAAATTTACAAGAGATTAGTGTGCTATGA
GAGGTAAAATAATATGCATAAAAAGATTGAGAGGGCAGGTTTGGATTTTGGGCTATGAGTATTA
ATAATAGCTTTCATTTGTTGTTGTTGTTGCTGCTGCTGCTGCTGCTGTTGTTTTTGAGACAGAG
TCTTGCTCTGTTGCCCAGGCTGGAGTGCAGTGGCACCTTCTTGACTCACTGCAACGTCCATCTC
CCAGGTTCAAGTGATTCTCATGCCTTGGCCTCCCAGGTAGCTGGGATTACAGGCGTGTGCCACC
ACACCTGGCTAAGTTTTGTATTTTTAGTAGAGACGGGGTTTCACCGTGTTGGCCAGGCTAGTCT
CGAACTCCTGGCCTCAAGTGATCCACCCGCCTTGGCCTCCCAAAGTGCTGGGATTACAGGCGTT
AGCCACCACGCCCAGCCATTTGTGAGCACATTATGTCAGGCACTGTTCTGAGATACTTAGATGT
TAGCTTGTTTATCCATGACAGCTCTATGGAATAGGTACTATTATCCTAGTTCTACCTATAAGGA
AACTGAGACTCGGAGAGGTTAAGTAACTTGCCCAAGATTTCTCAGCTAGTGAATGACCAAACAG
AGACATAAGTCCAGCCTGGCCTTGGAGCCTTTTGAGCACCGGATCTCATAAAAGAGGGGACTGG
AAGAGAGGACTTGACTCTAGTTTTCAAAATACATATAGCTCTTTAATTACATTGAGGTCAATTA
GTCAATTAAAAGATATTTTAACTTTATTATTTTTGTGTTTACAAGATGGAAAGTTAGACTTGTT
TGAATCTTCTAATTCCACATACCATTAGTCTCTTGAGAACCCAAGGGGAAGCTGTATCATGACC
ACATTGTTTATTTTAGGGAAGGTGGTGTTTCCCATATGACCTCAGCAATGTCTAAGTTCTTATT
AGGCAATGTATAGTTAGGATAGGAGTATCTGGGAGATTGCGTTATTTCTTACAGATACTATAAA
TCCCATTTATTGGAATCCCCTGCAATTATTTTTATATATTCAGGCCAATCCTCACCTAATCTGG
ATTTTGAAGCCCTGCGTTCATAGCTGCATAGGTATGGGCTCAAACATTCATGTTTTAACCGTTG
GTATTCGCAGATACCTTGCTGTTTTATATTGGGTTCTGAATCCACACTGGTAACCACCTTTTAC
GCTTGACTATTGTTGCTTTGAGGATGTTAATTATGAAAGGTTTCTTCCTCCTAATTTCCTCATC
TTTCCGTAGTTGATGCCTCAGATTTGGCCTGGATAGGGGAATCGTGAGGGGAAAGAACTAGTTG
CACAAAATGATCTTTTGATACTGGGATATTTCAGATGTATGGTATCTTTGCCAGAACTACAGTG
TCAGTAAAGAGGCCTGCTAAATTTGAACTGTGACAATCAGAAAAGTTCAGCTAAGGCCATGTAA
GGGGTTAAAAAGATATTTGGTTGAGGGAGGAGGATATGCTTTCATCTTTTGCCATACTTTTTCA
TGAAATTAAATCTTAGTAGATTTATGGCCAAGAAAGAGTAGAATATATTTTTATGCAGTACTTA
AGGGGTTTTGACTGATGTTTTTTATTATTATTATACTTTAAGTTTTAGGGTACATGTGCACAAT
GTGCAGGTTAGTTACATATGTATACATGTGCCATGCTGGTGTGCTGCACCCATTAACTCGTCAT
TTAGCATTAGGTATATTTCCTAATGCTATCCCTCCCCTTCCCCCCACCCCACAACAGTCCCCAG
AGTGTGATGTTCCCCTTCCTGTGTCCATGTGTTCTCATTGTTCAGTTCCCACCTGTGAGTGAGA
ACATGCGGTGACTGATGTTAAATGACTTTACGTACATACAGAATTAGTGGGACTAGACTTTGTG
TTTATGTGTGTGTGTTTATGTGTATGTATATAACTATTCCATTAAGCAGTTGCCCTCCTTGGTG
ACTGATTGCTCTATCAGCTTAGGAATCTTTTGACTGAATTTGTCCGTATTTGAAAGATCTCAAT
CTGTGTTCTGAATCTTTACTCATTTACATATACTTAAATCTTGAAACCCAGTATAGCAAAACTA
AGCCAAAACAAACAAAAAAATCCATCCCTTGATAGGAAGTCAGTATATCTGGGTTTTAGTCAGT
CCTTCAACACTAGTTCCAAAATCTTGGGCAAGTCAGTTAATCACTCGGAGGCTGCTTAATTTTT
TTTTTTTTTAATTCTTAGTATATTATCTGTCTTGATTAGGTAAAGCCAAAGTTGTTATAAGACT
CAAGGTACTATTGACCAAAATGCTTCAGAAACTATAACATTACATAAAAGTACAAGGAATTATT
ACATTTTCTCAAATGAAGAGATCATTTAAGAAAAACAAAATGCTGGAAAATAGTCCTTAAAAAC
TTTTCTATAATAATTACTCTTCTGATGAATTTTACATTGACACCTAATTTGAAGCTTTGAATAT
TATTTTTTGAACAATGAATTGGGTTTATATATTAAAGTGTTCTAACCAAAGGTTTGTTTAATTT
ATTAGAGTTATTAAGCCTACGCTCAGATCAAGGTAGCAGCTAGACTGGTGTGACAACCTGTTTT
TAATCAGTGACTCAAAGCTGTGATCACCCTGATGTCACCGAATGGCCACAGCTTGTAAAAGGTA
ATTTTGAATTATTTTACAGCCTTTAAAAGGCTGTCATTGTAAAGTGAAATACATACTGTAAAAA
TGAAGACAATGTATAGTTGGCAGGAATACTGCTAAGAATAGTGGGCCTGAGAGTTGACCTACTT
CATTTAATCTCCTTAAAATATTTTAGTGTCTTTTTTTCTCATTAAAAATATCAGTAGCCACTAT
CAAAGACCTATCTGTATGTATAAACAGGAAAGTATTTCTCAAAAAAACTTCAAGGTTTTAAACA
TTTTACTATAAACATTATAGAAAACATTAAAAATTTCTTAATATGCATTTTAAAGTATGCCTTT
TATGTGGTGAACGTTTTAACTAAACATTTCTCTAGAAGTTTTTATAACATTAATAAAACTAGTA
TACTTTTCTCTCCTAGAGAGTTACAGTGGAGGTAAAAGGAGTGGCTTGCAGGATGGAGAAGCTG
CACCAGTGTTATTGGAAGTGAGCCACCATTTGAATTTGCTAGCTCATGCTGCAGTATTCAGATT
AGTGGGTGTTTTGTGATCATATTTTCTGCGATTCAAATCAAGACCTAAAATTGATTCTCATTCT
GAGATTTACATTTTATTATCCAATGTTATTTGGTTATTGTTTCAGTTTTAAAATTTCTGACTTG
TTATTCTGACACACTTTAGGATAACCTAAGTCAATAGTTTAATACAAAAATTTCCCTTTGGTTA
TATGTTTTGAATTAGGTTATATGTTCTGCATTTACAAGCACATACAGGATTCTTACTTCCAGCA
ACTTGGCAGTAAAAGAATAAGTAGACTCTTTAGGAGCCAAAGTCAAGCTTGGGAACTGTTGCTA
GGTGGTATATATTTCATAGTTTATGTTTGTTTCCAAGACAAGGTCTCACTCCATCGCCCAGGCT
GGAGTGCAGTGGCACGATCACAGTTCACTGCAACTTTGACCTTCTGGGCTCAAGTGACCCTCCC
ACCTCAGCCTCCCAAGTTGCTGGGACTAAAGGCACCCACCACCACACCCGGCTAAGAGACGAGG
TTTCATCATGTTGCCCAGGTTGACCTTGAACTCCTGAGCTCAAGTGGTCCTTATGTCTTGACCT
CCCAAAGTGCTGAGATTACAGACATGAACTACCACACCTGGCATATGTCATAGTTTTGAGAGTT
TGAATTCTAACCTCGGGACTTTGGTAACTTATTTATAATATTCTAGAAATATACTGTTATTAGG
ATGAGTAGAAATTCATTCAGACAGAAACTATTATAAATTACTGCAAGAAAAATGTCTGCTAGTT
CTGCATATATTCATTTTTCTCTTGATTAAAAAACAATTTCCAATTTGAGTAGAACAACTTTGCT
ATGTTTAATAGACCCAGGACAGATTTATTTTAAGGTAATAATCTTTCAATCCTTCTTCTTCTAT
ACTGGATTGGTTTAAATTACTTAGCTTGTCTTATCAATGGTTTTTAATAGCCTACTTTAATATA
TGTAAAAAGTAGTAATTAATATGTTACAGTTTGGTGGCAAACATTTTGATGCCAAAGTTCACAT
ATTCTCTTGCTTAGGGAACAGAAATTGGGAAGATAGATGTAGTTATCATTTGATAATGTATTTA
AACTCTATCCCATTTCAGGATCTCAAAACTTTGTAACAGTTACATATTGTACTTCTAAGACTTA
GATTTGTTTTTCTTCTTGTGTCTTACCTTTTTGCAGTAATATTTTTATTTTCCTATCATCATGT
CCTCATTTTTTCCCTCTCTTCTCTTTCCCTGTTAATTTTGAAAGTTTTGCTGCCTACGTGTTTG
AAAACTTCTAATCTGCCTCTCTTCCCTCAGTGCCAGCAGGTTTATTTTTTGTTTTGCAAGCCAG
CTCTGCCTCCTTACAGTATGACATCTGATGCTGGAGGGTCGCACTTTCAAAAATGAGTCAGCTG
GTACATGGGGTTATCATCAATTTTTAGCTCTTCTGTCTGGGAGATACAAGTTTGGAAGCAATCT
TGGGGTACTTACCCACAAGGCTGGTGGAGACCAGGTGTGTCACATAGGTGATTTGCTTGCTCCC
TGGGGGAGAGGGTGGAGTGAAATTTTTGCATTTGTGTCACAGCCAAGTCACTGCCACCTCCAGC
ATGCCCCAGTTTTTAGTTGTTATGTAATTTTGTGCAAAATTGAAAATTTAAATACATTGCAAAG
AAGATCTAAAATAAATATTTGAGCTGCTGGAGTCTTTTTTTTTTTTTTTTTTCCTTTTCCATTC
TTGTCCTTGGAATCTGAATGAATTGCAGTGAGGGTAATTTTGAGAGTCTGATAAAGAAGTGAGG
GGTTGTTTTTTGTTTTTGTTTTTGTTTTTAGCTAAGAAATTTAGAAAACAGGTTTGTGTGTGTG
CGTGTGTGTGTGTGTGTGTGTGTGTGTGACTGATTTTAAGATTCTTGTTAGAGTTGCTTAAAGT
TGGAAGCCTAAAGTCAGTGAGAAGTTCACAAATCAGGACTTTGTAATGCCAATTTGAAAATTTA
GCTTTTGACCTCAAAAAACATTTTTTATCTAGTTTGGGAATTTCTAAGTGTTAGAAATCAATAT
TATATTGACTCATTCATTGTTGCTGATGCTGTTTATAAAAGTGAATAGATAGGGAAATACTGAT
TCATATCTCGTGAAATGAAAATGAAAGGCTTTTTAGTGACTTAGAATTTTAAATATTTCTACAT
GAGAGAGCAGTAGTATATTTAGAAATAACAAAGTAACTGGCAACTGTTTAAAACTGAAGTTAAT
TCACAGCTATCCAGTGCAAAACTTCACCTCAGGTGATACACTTTTGACAGGTAATACATACAGT
AAGTGTATTTTTAGGGAAACAGTTTCATTGTTGAACCAAGATAATCATCATTAGAATGTTGTAT
CTGATTTAAGTGTCTTTAAACTTACCAAGGTATTAGATTTTAGTTTGAATTGTCTGGAGTAGCG
GTAGCGGTTGGCACATTTGTTCTTAAAGGGCCAGAAAATAAATATTTTAGGCTTTATGGGCCAT
GTGGTTTCTGCCACAAGTCCTCAGCTCTGTTCTTTTAGTGTGAAAGCAGCCATAGATGATACCT
AAATGAATGAGTATGGCTGCATCCCAATAAAACTTCATTTACAAAACTACATGGCTGGCCTAAG
CTTTAGTCTGTCTGCCTAGGGAGTAGTTTACTGAGCCACTAATCTAAAGTTTAATACTGTGAGT
GAATACCAGTGAGTACCTTTGTTAATGTGGATAACCAATACTTGGCTATAGGAAGTTTTTTAGT
TGTGTGTTTTATTACACGTATTTGACTTTGTGAATAATTATGGCTTATAATGGCTTGTCTGTTG
GTATCTATGTATAGCGTTTACAGTTTCCTTTAAAAAACATGCATTGAGTTTTTTAATAGTCCAA
CCCTTAAAATAAATGTGTTGTATGGCCACCTGATCTGACCACTTTCTTTCATGTTGACATCTTT
AATTTTAAAACTGTTTTATTTAGTGCTTAAATCTTGTTTACAAAATTGTCTTCCTAAGTAATAT
GTCTACCTTTTTTTTTGGAATATGGAATATTTTGCTAACTGTTTCTCAATTGCATTTTACAGAT
CAGGAGAACCTCAGTCTGACGACATTGAAGCTAGCCGAATGTAAGTGTAACTTGGTTGAGACTG
TGGTTCTTATTTTGAGTTGCCCTAGACTGCTTTAAATTACGTCACATTATTTGGAAATAATTTC
TGGTTAAAAGAAAGGAATCATTTAGCAGTAAATGGGAGATAGGAACATACCTACTTTTTTTCCT
ATCAGATAACTCTAAACCTCGGTAACAGTTTACTAGGTTTCTACTACTAGATAGATAAATGCAC
ACGCCTAAATTCTTAGTCTTTTTGCTTCCCTGGTAGCAGTTGTAGGGAAATAGGGAGGTTGAGG
AAAGAGTTGAGTTTAACAGTCTCAACGCCTACCATATTTAAGGCATCAAGTACTATGTTATAGA
TACAGAGATGCGTAATAATTAGTTTTCACCCTACAGAAATTTATATTATACTCAAGAGTGAAAG
ATGCAGAAGCAAATAATTTCAGTCACTGAGGTAGAATGGTATCCAAAATACAATAGTAACATGA
AGGAGTACTGGAGTACCCAGGTATGCAATAGGAATCTAGTGTAGATGGCAGGGAAGTAAGAGTG
GCCAGGAAATGCTAAGTTCAGTCTTGAAATGTGACTGGGAATCAGGCAGCTAGAAGCATGAGCG
GCTTATTCCAGGCAGAAGTATGAGCCAAAGTTAGAAGCAGCATAGGGAGTAGGGGACAATAGGC
AGTTGAGGACTTTTAGATCATAAACTAGCTGAGAGGTAGGAAAATGAAGAAGATAAGATGTGAT
AGAAGGTATTAGGGAGTCAGCAAAGAATTTGCTCAGTAGGTAGATAGTAAGTTTGTCAAACGTG
TATTTTTAATAACCTTGCAAGAGAGAAAGGATTGAAGAGAAACGTGTGGTGGTAAGGTAGTTCA
GTCAGTGAGAGCCTTGACAATGACAGTGGTATAATAGGAATTCAAAATAAATTTTTCTGGATTT
AAGACAAATATTTAGGACATGAAGTAAGTAAGACTTTAGTGATTTATTGTGAAGACTAAAGATG
TTTCAGATAATAGAGGTAGTAATTATCAAAGCAAAGTTTTTCGAGGTATTAAGGGTTGGAATGA
AAAATACAGGTGTATCTCTCATACAGGTAATATAAAAAAAGAAAAATGCAGATATAGTGATTGA
CCTTGAATAAGAGGAAAGATGCCCATCTAATGAAATTAGAACAAGGAAGTAAAGATGGATGTGT
GATATTAATTGTAATTGTGGGTGAGGGTGGAAAAATCAAGATTTTTTTACTGAAAATATTGGAA
CCTTATTTAAATGCCGGTATATATTGTGAATCTGCAAGAAACGACTATAAAATGTATTTCTCAA
ATTTATGTGACCCAACCATGTGACTTGTCTTTTAAGCTTTAAAAAATTGTAGTAAAATACACAT
AATGTAAAATTTTCCATTTCACCATTTTTAAGTATTTGGCTCAATGGTATTAAGTATATTAATA
TTGTTGCACAGTCACGATCAGAACTCTCTGTTTTGCAGAACTGAAACTCTGTACCCATTAAACA
ATAATTCCTCATTTCCTTATCTTCACAGCCTCTGACAACCATTATTCTACTTTGTGTCTAATTT
TGACTGCTCTAAGTACCCCCTATAAGTGCAATCATACAGTTTTTGCCCTTTTATGATTGGCTTA
TTTTACTTCGCAGTGTCATCAAGTTTCATCCATCTTTAGCATGTTTCAAAATTTGCATCCTTTT
TCAGGCTGAATAATGTTTTATTGTATGTATATACCACATTTTGCTTATCCATTTACCTGTTGAT
GAGCGCTCAGGTTGCTTCCATGTTGTAGCTATTACAGATAATGTTGCTATGAACAGGAGTGTAC
AAATATCTTTTTGAGACCCTGCTTTGAATTGTTTTCCCAGAAGTGGAATTGTTGGATCATATGG
TAATTTTATTTTTAAAATTTTGAGAAACTGCCATACTCTTCCAGAGCAACTGTACCTATTTGCG
TTTCTACCACTAGTGCACAAGGGGTCCAGTTTCTCTACATCCTTGCCATCACATATTGTGTGAG
AGTGTGTGTGTGTGTGTACTTCAATAGTAGTCATTGTAGTGGATATAAGGTAGTATGTTACTGT
AGGTTAGTGACCACGAAACTTTTTATGAAACATTTTTTAATAATATTTTGTGGAATACAAGGTT
TGTTTAGAGGTTTTGAGAAGACTGAAAACTTAGGAAGCTACTGAGGGGAATGGGTGAAGAAGTG
GATCAGAAGTTGAGAGTCTTGACTGGCTGGCTACCTGAAATCAGAAACCGTATCAGAGTAGGAG
AATCTCATTCCACTGAGTGACTAGTTAAGATTAAAGACTTTATCACATTTGAAATTTAAAGACT
TTTCTCCAATAGCCAGAAGATTATGCATGGTATTTTAGGGTTGGATATTAGCAGAGCTGATGGA
ACAGAATCATTAGTGGCATGTGCATAAGATGTTAACATTGGAGTATTTGAATTTATATGCTGTG
GATGATGGCCATGATTGGAAAGGTTGGGATGCCAGACCAAAGTGTTGAGTTGTTTTAAGAAAAG
CCTGAGGTCTAGATAACAGCAAGATGGCAGACTAGCACTTTGCAGCACTCATCTCATTGTAGAA
CCATCAATTTGAACAACTGTCTACACACAAAAATATAAGAGCTAAGAAACCAGACGAGAGATTA
CCTGAATGTGGCACACAGATAAGAAAAGACACATTGAAAAGGGTAAGAAGGACAGTTTTACATT
ACTTGTGTCACACCTCCCTCAGTCCCTGAGATCCCAGCATGGAGAGAGAGACTGTTCACCTGGG
GGAAGGAGGGGAAGTGTACACCAGACTTAGACCCTAACATTGAGCTCGCCCCAGTAAAACTCAA
TGCTAGGCAGGCCCCCATGGCCCCAAACTTCAGGCCAATACCCACAAACCAAGCTTGGAGGCCC
ACCCCAGCACCAAGCTGGATCCCACAGCCCTAGGCTCTAAGCCTGTCTAGTGCTAGGGTGGTCC
CTGCAGCCCTGCCCTCTAGATCGGCCCCTGTGGCCTCACATCACTGCAGAACAAGGGTCCAAAC
CCAGTACTTGGCCAGCCCCTTGTGATCCAAATTCTAGGCCAACATCCACCTACCCAGCCTTCAC
ACTGGTCCTTGCAGATCCAAGGTTCAGGCTCACTCTAGTAGACCAGGATGCCAGGCCAGCAGCA
CCTGTGTACCAAGGCTCCAGGACCTACCCTGCTGACCCAGACTCCAGGCCAGCCCCGGTAGACT
CAGGCTGCATAATCTCTGGACAGGCTGACTGGTGAAGGGCTTTACCCAATAAAGTCACTCTGCA
AAGACTGGAACAAGTCTCAGCTTCTTCAAATGTGCAGGTGCCAACACATGACTACAGGGATCAA
GAACAGTCAGGGAAACGTGACACCAGTCAAAGGAACACAATAAGGCACCTGAACCAACCATAAA
GAAATGGAGATATATGAACTGCCTGACAAAGAATTCAAAGTAATTGTTTAAGGAAGCTCAATGA
ATTTCAAGAAAATGCAGATAAACAATTCAACAAAATCAGGAAAACAGCAATCAAAATGAGAAAT
TTTGAGATTGAAATTATTTTTTAACAAATTCTGAAGCTGAAAATGCAATAAATGGAATGAAAAA
TGCAAAAGAGCATTAAAAGCAGAATTGAGCAAGCAGACAAAAGAATCTGAACTCAAAAGACAGG
TTATTTGGCTAGACTAAGGAAAAAAGAGATGACTCAAGATAAAAAAAATAAAGAGTTGTGTTTT
TTGATGATAAAAGCAACAAGTCTTTAGCTAGACTAAGAAAAAAAGAGATGACTCAAGTTAAATC
AGAAATGAAAGAGGAACCATTGCAGCTGATACCAGAGAAGTACAAGAAATAAGAGGATCATAAG
AGACTGAACAATTATATGCCAACAAATTGATTACCATAGAAGAAATGGATAAATTCCTAGACAC
ATGCAACTTGTCCAAGCTTAATCATGAAATGAAAAATGTGAACAGTCCAGTGACAAGGATTGTA
TCAGTCATAAAAATATCTCCCACCAAAGGAAGCCCAAGACTTGGTGACTTTACTGCTGAATTCT
AGCAGACATTTAAAGAAGAACTAGTACAGATTGTTCTCATAATTTTATGAGAAATTTAAGAAGA
GGGAGTAATTTCAAACTCATTTTGGGAGATGAGCATTACCTAGATACCAAAGCCAGACAAGAAC
TCTCCAGGAAAAGAAAATTACAGACCCGTATTCTTAATTTAGATGCAGAAATTCTGAAGAAAAT
AATAACTAAATTCAACAGTGTATTAGAAGGATCATTTACTGTGATCATATGGGATTTACCTCAA
GGGTACAAGGATTATCCAACAGACCTGAATCCATAAAATGTGATATACAACATTAGGAGAACAA
AGGCCAAAACCATGTTATCATTTCAATAGATAATTGAGATAATTGAGAAAAAGCATTTGACAGA
ATTCAGTATTCTCTTGTGCTACAAAATTTTAAAAAATTAAGTATAGAAGGAATGTACCTCTATA
AAATAAAGGCCATATTGACAAACACACTGCTAACGTTATACTGAATGTTGAAAAGTTAAAAGCT
TTTCTTTTGAGATCAGGAAGAAAACGAGAATGCCCACTTTTGCCATTTCTATTCAGCACAGTAC
CGGAAGTCCTTGCAAGAGCAATTAGGCAAGAGAAAGAAAAGGCATCCCAGTAGGAAAGGAAGAC
ATTGTTGTCCCTGTTTGAAGATGACATGATCTTGTATATAGAAAAACCTAAAGACTCCACCAGA
GAATTGTTAGAACTGATAAATTCAGTAAAGTTGTAAGATATAAAATCAAGATACAAAAATCCGT
AGAATTTCTGTACATTAATGACAAATTATCTAAAAGAAAAATCAAGAAGACAATTCCATGTATA
ATAGGTACCAAAAACAAAACATGCAGTGGCTCCCAGTATCAGTGGTTCTGCATTTGCAAATTGA
ACCAACATTTGGATGAAAATATTTGAGAAAAAAATACAACAATAAAAATACTGTTTTCTTATAC
AATAAGGAAACTACAGTATAGCAACTATTTACATAGCATTTACGTTGTATTAGGTACTAAGTAA
TGATTTAAAGTATACACGAGGATGTGTGTAGGTTGTATGCAAATACTATACCATTTTATATAAT
GGACTTGAGCATCCATGGATTTTGGTATCTACAGGAGATCCTGGAACCAATATCCTGCAGATAC
CGAGGGATGATTGTACTTAGAAATAACTTTAACCAAAGAGTTGAAAACTATGTAACATTGATTA
AAGAAATTGAAGATGATACAAATAATGGAAAGTTATCTTGTGTTTATGGATTGGAAGAATTAAT
ATTGTAAAATGTTTTTATTATCCAAAGCAATCTGCAGTTTTAATGCAATCCTGATCAAAATTCC
AATGTTAAATTTCACAGAACTAGAAAACAGTGCTAAAATTTATATGGAACCACATAAGATCCTA
AATAGCCAAAGCTGTCATGAGTGAAAAGAACAAAGCTGGAGACATCACACTATCTGATTTCAAA
ATATAGTTCAAAGCTACAGTAATTATAACAGCATGATAGTCACATAGAAGTAGACAGGCCAGGC
ACAGTTGCTCACACGTGTATTCCCACACTCTGGGAGACTGAGGCAGAAGGATTACTTGAAACCA
GGAGTTCGAGAGCAGCCTGGGCAACATAGTGAGACCCTGTCTCTAAAATTAAAAAAAAAAAAAA
TTTTTAAAGCCAGGCGCGGTGCTGTGTATCTGTAGTCCTAGCTACCCGAGAGGCTGAAGAAGGA
GGATTGCTTGAGCTCAGGAGCTTAAGGTGGCAGTGAACTGTGATTGCATCACTGCACTCCAGCC
TGGATGACAGCGTGAGACCCTGTCTTTTAAAAACAAAAGAAACAAAAAAACAGACACATCGACC
CACAAAACAGAATGGAGAGCACAGAAATAAATTCGTGTACTATCAGTTGATTTTGGACAGGGTG
CAAAGAACACAGCGGGGAAAGGGAAGTCTGTTCAATAGGTGGTGTTGGGAAAATGGAATATCTA
CATGCATGAGAATGAAATCAGACCTGTATCTCACATCATATACCAAATTCAAGTTAAAATGGAT
TAAGGTCTTAAATATAAGACCTGAAACTGTAGAACTACCAGAATAAAGTATAGGAGAAATGTAC
ATGACATTGGTTTGGACAGTGATTTTTTTGGATATGACCCCCTCGAAGCACAGGCAACAAAAAT
AAAAATTGATGAATGAGATCACATCAAACTAAAAAGCTTCTGCATGGCAAAAAGAAAAAATCTA
CAGAATGAAAAAACTCACAGAATGGAAGATAGTATTTCCAAACCATATATCTGGTAAGGGGTTA
ATATGCAAAATATATAAGGAACTCAGACATCTCAATAGTAAGAAAGCACCTCACTTAAGAAATG
GGCAAAGGACTTGAATAGACATTTCTCAAAATAAGACATACAAATGGCCATTAGAATGGCTATT
AAAGTATGATGAGGATTTGCAGACAAGAGATATATTGCGCATGGCAGGAATGTAAATTAGTACA
GCATTTATGGAAAATAGCATGACGTTTGCTCAGAAATTAAAAAATAGAACTACCATATGATTCA
GCAGTCACACTTGGATATATATGTATTTTTATATATATATATATATATATATATATATATATAT
ATATATATAAATAAAAGAATAAAATCAGTGCGCTGAAGAGATCTGTACTCCCATGTTCCTTTCG
GTATCATTTGCAATACCCATGATACAGCATCAGCCTAAGTGTTCATGGATAAAGAAAATGTGGT
ATGTATACACAGTGGAATATACTTCAGCCATAAAAAGGAGGAAATGCTGTCATTTGTGACAGCA
GGGATGAACCTGGAGGGCATTGTTGAAAGTAAGCCAGCCACAGAAAGGCAAATACTACATCTCT
CTTAAATGTGGAATCTTAACTCAGGGATGTAAAATGGTGGTTACCAGGGGCTGGGGGTGAGTGG
ACTGGGTTGCAGAGGTGTTGGTCAGAGGACACAAAATTTCAATTAAGAGGAATTTGTTCAAGGT
ATCTATTGTACAACATGGAAACTAGTTAATAACAGTGTATTTTTGGAAATCACTGAGAGTAGAT
TTTAAATGTTCTGCCATAAAATATAAGTATGTGAGGTATTGCATGTATTAATTAGCTTGATCTA
GCCATTCCACAGTGTATACATATTTTGGAACATCATGTGAATACAATAAATATGTATCCTTTTT
TTTGTTTTTTGTGTTTTTTTTTTTTTTTTTTTTTTTTGAGACAGAGTCTTGCTTTGTCACCCAG
GCTGGAGTGTAGTAGCATGATCTTGGCCACTGCATCCTCTGCTTCCTGGGTTCAAATGATTCTC
CTGCCTCAGCCTCCCAAATACGTATCATTTTTATTTGTCACCAAACCTGGTGTCTTGACAGGTG
AAAGGAGATATGAGTATGAGTGAAACTCTTACAAAGATAGCTGAGATGATCATTGTCAGAGGTT
TTAGTATGTCTAACAAAGACAAGGGAGTGATTTGCTGAAATGAGGAGGTGAAGATGTCTCAAAT
TTAGAAAGGCAAATATTTTGGGGGCCTAGAGTATTGGGATAGAACCATGTACAGGGACATTGAA
GTCACTTTGCTGATGATGTAACTGGGGGCAAAGTGGAGGCTTAAATATGTGGGTTTAATTGACC
AGAACAGGGAGGGAAAGAGAAGGCATTCGGTTAGATGACATGAGTTTCAAATTACAAGCTGTTT
TTTTGTTGTTTTTAATACAGGGAATTTTGCCGTTTATTTTTAATACAGGGAATTAGAAGAATAA
TGGTGGACTTAGGAGTTTTAGGAGTCACATAAAAGGGTTGCATGATAGGCAGTGCCTTTGGGGA
GGAGCCAGGCTTTCATGAAAGCTAAGAAGTTGAGGGGCCATTATGTAAAGTAGTTGAAGATGGG
GGATAGCCTATGGGTCTTCAGAAAGCCTCTGGGTTTCTAGAGATCTTTCTGGAAGAGGTTAGGA
GGATGAGTCAAGTTTAGAATGGAAGAGGATAGATGAAGCTTGCATTTGGTAGTAAAAACCAAGG
ATGAATGTGTGAGGCATGATTATTTGTCACAAGTTTCCAGGTGACACTCTGGCACTCAGTCCTT
GTTGAATTAAGCAAGCTTTCTTACTGTTTTACTTGGCTGTGTTAAAGTAGAGTAACAGGAAATT
AGACTTCCCATAATGTCTCAATAGAGCTGTTTTAAGTTTCTAGCCATAATTGGTATGGGAATAT
AGCTCTAAACCTTCAAGGCTGCTACCATTTCTCACATTCCTATATCTAGAAACTGTAGCCAGGC
AGCACTTTCAGTGAGGTGAGTGGGTCCAGGTTTGTTCAATTACTTTTGTACCAACCTAATACCT
TAATAGTATTTGTCCTAATTTACAAAAATAGGTAATGAAGGATTCTTGGTTTTACCTCAGAGCT
AACTGTCCATTAATGGCAACATCCCACATTTGTTGAAGCATGATTTTGTTGTTTGTACTTTGAC
ATGCATTTGATATTTTAAGTAACGCCTTGAATGCATCTTAAATAATAATTTATATACAGTTAAC
ACTCCTGTTTGTTTTTAGGAATTCTTAGTGTTGCCAATTTCCTAGGATGTAGAAGAGCATAGTA
AAAAAAAAAATTATTAATACTCTTAAACAAGTGTTTTTAACAACTGGACACCATTTTGTAGTTA
AGCCTAGAAACAGAACTAATGCAATAACCCAGATCTTTTCATTTATTCGTATGTTTATTTTACC
TGGATTTACTTTAGTTTTTTGTTTTGTTTGCTTGTTGTTTGAGATATTGTCTTGCTCTGTCATA
AGTGATACAGCTAGAGTGCAGTGGTGTGATCAGAGCTCACTACAACTTTGAATTCCTGGGCTCA
AGTGATCCTCATGCCTCAGCCTCCTGAGTAGCTGGGACTACAGGCATGTGCCACCATGCCAGGC
TAAAACAGTGTTTTTGTTTGTTTGTTTGTTTTTTGTAGAGACAGGGTCTCACTATGTTGCCCAG
GCTGGTCTTGAACTCTTGGCCCCAAAGTAATCCTTCTGCCTTGGCCTCACAAAGTGCTGGGACT
ACAGACGTGAGCCACCGTGCCTGGCCATGGATTTTGTTTAAATTTATAAATACCGTGTTGTAGA
ATCTCTTTGTATGTATTAATTTAGACACCTTAGTTTTCTTAATGATTTCATACCAGTTTCTCAG
TAACTTCAGATTTTTTTTTTGAGATGGAATCTTGCTCTGTCACCCAGGCTGGAGTGCAGTGGCG
CAATTTGGCTCACTGCAACCCCTGCCTCCCAGGATGAAGCAATTCTCCTGCCTCAGCCACCCGA
GTAGCTGAGATTACAGGCATGTGCCACCATGCCTGGCTAATTTTGTATTTTTAGTAGAGACAGA
GTTTCACCATATTGGCCAGGCTGGTCTTGAACTCCTGACCTCGTGATCTGCCTGCCTCGGCCTT
CCAGAGCGCTGGGATTACAGGCTTGAGCCACCGCACCCAACCAGATCAGATTTTTTATGTTTGG
TATTTACTCTTTACCTCTAACACTAAGGCTATGTTTCCAGATGTATAACTATAAATGGAGCAGG
GGTAAATACAACAAGTGATTTATGGTTCAGTAAAGCAAGTTAGTTTTAATAAATGCTCAGTTTA
CTGTAAATGTCAGAAATGCCCCAGGACTCTGTCTATTGACAACTCTTCTATTTCCATCGTCAGC
TCTTGCCCTCTGAACTGTAGACTGATTTATCCAACAGTTAAGTCTGGTTCTCAAACTTAACCTG
TCCTAAGTGGAGCTCCTGATTTTCATCATCCCAAACCTACGTCTCTCATAATATTTTGCCATCT
CAGTCAATAACAGCTTTTTTCCCCTCCTGTGCAGCCAGAAGCCTTGGAAGCATCTTTTAATTTC
TCTATGTCACATACTCAAATTATTGGTAAATCTTGTTGGCTACACCTTCCAAATACCCATAATT
CATTTTATTTTTTTTAACCATTATACTTCTGTTTACTCTGTTTAAAGGCATCATCTTTTGGCAA
AAGAAAGAAATTGTTTCCCAGGAGTCTTGTGCTTTACAGTGTATTCTCTAGTAGCAGCCGGAAT
GATCTTTTTAAAATCTGAGTTAAACCATGTTATTTACTATGCACAGAACTTTTCAGAGGCTTCC
CATCTTAAAAGTAAAATCTGGAGATTTACATGGCCCTGTGTGAACTGGCTTCCTTTCATTTCTG
TGATCTCTCTACCGCACTTGCTCATCTCTGTCTAAAATGGCTGTATCTTTGTCATCTTAAGGCT
TGTAACTATTGTCTGTGACCCTGTGACCCTTCTCTGTGATAATCCATGTGGCTTCCTTCCTCAC
TTGATTCAGAGAGCTCTCTGCTCAAATGATGCTTTTCCTGACCGGCTGCATCAAAGAATGATCA
TGTTCTTCTGCATTCCCCTTAACTTGCTGCATTTTTCTTCATGGGATTTAATGTCATTTGACTT
CTTGCATATTGATTTCTTTGTTGTTTTGCTTCTTTACCACTAGAATGGAAGCCTAATGACAGTG
CCTGGCATACAGTAGGTAGTCAGTAACTACTTCCTACATGAAAGAATGAAGCTAGTAAATTTAT
GCAAAATGTTTGTTTCTGCCTTTTTAATTATCTTGCAGAAATGTAGGAAGAATTTTTTAGGATC
AGTTTATGTATATATATAAACTGATAAGTATGTACACAGCCTGTAGAATGTGGGTAATACCACC
TATCATCTTTTTGAGCATTTACCATGTAGCAAGTGACTGTGCTCCATAATCCACATGCATTATC
TTGAATTTGATAGGAGAGCTGAGCTTTTTAATCAGAGTAGACCATAGAAATATAGCTTGGATGC
TTTTATTTCCTATATGACTTCTTCGGTGTACTTACACATCTATAAGCATTTATGGGTCAAGTAA
CATACTGAAATATTTTCTGAAGTAAAAGAAATCTCTTTAATCAAATAAGTGGTTCTCCTATAGG
TTCTCATATCATATTCTTGGTCTCCAGATTGAGAAGGTCCTTTTGTATGCTTCCAGTATACTCG
AAACCAGGAGGTAGTCTGACATCTATTTCCCTTCAAGGTCAAATAATTAGAGAAAGGTTTAATT
GACACTGTTATCGAGACAGTATCAGAAGAGAAATTGAAAAATCTCTGTTGAGACTCTGTAATTT
GCTAGGTTCTGAGGATACGACAGTAAATAAGTTATTAGGTCATATACCTCAGCTTCCCATTTAA
TTAATAGGCAGTTATTATATATAATAGAATAGGTACTGGTTGGTATGAGACTACAGAGGAAGAA
TACCTAATTATCTTAAAAGTGGTATATAAGCTGAAACCTTGAGGATGCTTGGGAATAGCTAAGA
ATCATAGTGTTCTAGGCAGGAAGAATAACACCTGGAATCAGAGAAGACAGCAGAACAAGTCCAC
TGAGTAGTAAGAAGTTCCGTATTGCAGTAATGTAGAGTTAGCTGATGGGAGAAGGTGTGAAAAA
ATGCATTTGGATATAGAAAGCCAGGAGCACGCTAAGGCCCTTTGCTTTCTTCTGACAGACACAG
GAGCCCATGAAAGGCATCACGTGTGGAAGGAAGTGACACGAGTCATAGTCTGCCCTGCTTTTCT
ACCACCTGAAGCATCATATAAACCTATATCTTCTGTTTCAAATGTAAGGAAACATACTTCTAGT
TCACAGTAGGGATAGTTGAATGGATGAACTGTTTCTGGCTGAATCTTCTCAAAGTAGAATGAGC
AAAACATGGCTATATTATGAGATTATCTAAGGAAAGATTTTTATAATCATGTGAATTTTGAATT
TCTTTATATCTTAATTATTCCAAGAGATTTTTGGGAACATTAATGAGAATAGTTTACCTTTAGT
ACATAGAACTTCTTGTCAGCTATAAATGAGGATCAAACCCAAAAGAGATTAACAAAAATAAAAG
GGATGCTATTTCCCCAAATTAGCAGAACTTTTTGATTCACCGAGTTCTTTGTTTTTTAACTGCT
GTCTGCCTCTTAACATAATTGAGTATTCAGTGATATAATATAAATAAATCACTTACTAAATGCT
GATGAACGCTTGTTTTTACTAACATCTCAGCTTGTAGCATATCTTGTTGCCCATACCAGAAACC
CTACCCATTACCTCTGGCTCTTCCAGCTTATTTACCTCTTACAAACTACTTATTTTAAATATTT
CCTGGGTCAATTTCATCTTTTTTTTTCTTCCTACTGCTACATTATTTGTCCGGACTATTACAGT
AGTCATTACCTAACTGGATTGCTTGCCTCCAGTTTTGCCCACTTTGGTTTCAGTTATTGAAAAG
GCAAATTTTGTCAGTCACTTAACCTGCTTAAAACCCTTCAGTGACTCCTTGTGTTTTAGTTCTT
ATCTAATGGTTTATGAATGTAGTGGGTTACGAATCTTCCGGAAGAGTGCATTTGCACTCCTGAA
GTGTGCATGTATTTTTATGAGGAGAAGATCTTTAGCTTTTATTAGATTACTCCTCTTTTGGCTA
AAAAAAGAATTACTTATCATGGCTATTGAGGTCTTCCATGATCTGGCTTTTCCCTGCCTCTCCA
TTTTCATATTTTTGAAACTTTTCTTGAACTTTAGGCTCTTGAAGTGCTTGGAATTGTCTTATAA
TATCATGTTTTCAGGCCCCTGTGCTTTTACTCATTTTCCTCTATCTGAAACATTCTTCCCCTGT
CTGAAACATTCTTCCCACCCATAGTGTCATCAGCTCCTAGTCATCTTTTGTGATTCACCTCTCC
TAAGTTGCCTCTCTTTTTATTGCTATTCCTCTGTAGTCATCTCTGTGATATTGAAATCGTGGCT
TTCTCTTTCTTTAGACATCATAATTCCTTGAAGAGCCCTTTTTATGTAGCCTGACAAGCAGAAT
TTATACTGAAGTAGACATTTGGAAGTCATTTAGTTATTGCTCAATCCTTGAAAGATGAAACTGT
CCAAAGGGAAATTTAAAAGGGAAAGTATCTAATAGCTGATCCCTGGAGAATACAAATGTCTCTA
GATTTGGAAGGGAAGTCTCTGAAAAACAGCCAGAGAGAAGGGAGGAAACCAAGAAATAGTGTCC
TAGACCCAAAAGTCAATAATTTCCAAAAGGTCTATATTTCTTAGGAAGGCCAAGTAAAATAAGT
AGTGAAAAGTGATGACCTGACTAGAGCAGATTCTTCATTTCCAGTTACAGAAGCTAGGTTATAG
GTACAGTAAGCTCTGGACATGTTCTTTTCCCACATGGAACGTTATTCGTGTTCTCCACTGCTGC
TTTGAACTATTCCTTTTACTCTCTCAGCATAAAAATCCCTGCTCCTTTGAAGCTTATGCCTTCT
GGTTTTGTCTTCCTCTGTCTGCCTTCAGTGAGCTATCAGTTTCTTCTCGGTTTTTGAGGACTGC
TACCTGAACTTATTCTTCTTCACTCTTTCCCAAATCTGCTGCCATCTTTATTTATATGATTAAC
CCCTCAGTCCCTAGCAAGTCAAATTCCCATCCGTTTTTATCTACAGTAATGTTCACCTCTGCAT
CACTGCTGCTTCCCATGTCTACAGGCATGCCAACTCAAAACTGTTTTACCCCAAAAGCTTCAGT
GAAAACACCTTACCTCAGGTTGCCGTCTTTGAATGCTTTTCTACTATCTTAAACTTGTTTTCTT
TCATTGCGCCTTTTCTTCCTTCTCTTAGAGACTTGTCCTATGGCTCTTCTACTGTCTTCCAGCT
ACAATTCCCTCTTGTTCCTGACATGTACTGCTCAGAGCCAGGAGATGGATAAGTAGTAGTATTT
CACATGCACTTTTTTCTCTGGGGAGCAGTAGTCATACAATAATTGATTAAGATAAAACAGGTTT
TTATCAGTTTATATAAGAGGTAAAGAAAACAATGGGGTAGAGTTTTAAAAATTGTCAAAAACAA
CACATTTTACCATATTAATGATTTGAAGTGTACGGTACAATTGTGGTAACTGTGCACATTGTTG
TGCAGCAGATCTCTAGAACTTTTTCATCTTGTAAAACAGAAACTCTACATTCATTGAACAACTC
TTCTTTTCCCCCTTCTTCCTATTCCTTGGCAAATGCCATTCTACTTTTTGCCTCTAAAATTTGA
CTGTTTTGGCTACTTCATGTCAGTGGAATCATTAAGTATTTGTCTTTTGTTGACTGGCCTATTT
CACATAGCAATGTCCTCAAGGTTCATTTGTGTTGTAGCATATTACAGAATTTCTTTCTTTTCTT
TTAGGCTGAATAACATTTTGTTATATATGTATACCACATTTTCTTTAACCATTTGTTGATGGAT
GTTTAGGTTCCTTCCACTTCTTGAATATTGTAATGTTGAAATAAACATGGGTATACAAATATTT
TTTTGAGAACCTGTTTTTAATATATTTGGAAGTATACCCAGGTGTAGGATTACTGGATCATATG
TATTCTATTTTTTTTTAGATTTTTTTTTTTTTTACAGGAGTTCTAGGTTCACAGAATTGAGAGG
AAGGTACAGAGATTTCCCCTGTACCTCCTTCCACACACATGTATGGATTCTCTCATAGCCAGTG
TGCATCTCCCACCAGAGTGGTACATGTCTTTCTGATGAACCTATATTGATACATCATAATTACC
CAAAGTCCGTAGTTTATATTAGGGATCATTCTTAGTGCTGTACGTTCTGTGGCTTTGAACAAAG
TACAATGACATGTATCTACCATTATATCATACAGAATGTTTTCACTGCCCCAAAATCCTCTGTG
TTCTGCCTATTTATCTTTCCTTCCCTCCCACTCCTTGGCAATCACCGATCCTTTTACTGTGTCT
CTAACTTTGCCTTTTCCAGCATATTATATACTTGTAATCATACAGTATATAGCCCTTTCAGCTT
GACTATTTTTACTTACTAATATGCATTAAAGTTTCTTCCATGTCTTTTCATAGGTTGTTAGTAC
CTCTCTTTTTCCATTTTCTGGACATATCACAGTTTGTGAATGAATATTCACCTAATGAAGGACA
TTGTGGTTGCTTCCACATTTTGCTAATTATGAATAAAACTGCTGTAGATATCGGTGTGCAGGTT
TTTGTGTGGACTTAGGTTTTCAGCGACTTTCTGTAAATACTAGGGAGTGCAATTGCTGGATCTT
ATGGTAAAAGTGTTTAGTTTTGTAGGAAACATCCAAACTGTCTTCTAATATGACGCTATCCTTT
TACATTTTTCACCAGCAATGAGTGAGTGTTTCTTTTGCTCTACATCTTCACCAGTATTTGGTGT
TATCAGTGTTCTGAATTTTGTCCATTCTGATAGGTGTGTAGAGGTATCTTATAATTTTAATTTG
TGTTTCTCTGATGCCATATGATGTGTGGGACATCTTTTCATATGCTGAATTGCCATCTGTATAT
CTTTGGTGAGGTGTCTGTTAAGGTTGTTGGCCAATTTTTTAATTGGGTTGTTTGTTTTCTTGTG
GAATTTTAAGAGTTCCTTACACTGTCTCTGTCTGTCTTGTCTGTCTGTCTGTCTGTCTGTTGAG
ACAGAGCCTCGCTCTGTCACCCAGGTTGCAGTACAGTGGCACAGTCTTGGCTCCCTGCAACCTC
TGCCACCTGGGTTCAAGTGATTCTCCTGCGTCAGCCTCCCGAGTAGCTGGGATTATACCCGTGC
ACCACCACACCTGGCTAATTTTTGTATTTTTAGTAGACACGGGGTTTCACTATGTTAGCCAGGC
TGGTCTCGACCTCCTGACCTCAGGTGATCCACCCACCTTGGCCTCCCAAAGTGCTGGGATGTGA
GCCACCACACCCAGCCTCCTTACACATTTTAGTTAATAGTTCTTTATCAGATGTGTCTTATGTA
AATATTTCCTCCCTATCTGTGGCTTGTCTTTTCATACTCTTGACATTGTCTTTTGCAAAAGACA
ACAATTTTTAAGAAACAAGTAGACAATTTTAATGAAGTCTGGCTTATCAGTTCTTTTCTTCATG
GTTGGTACCTTTGGTGCCGTATCTAAAAGTCATCACCAAACCCAAGATCTAGATTTTTTCTTAT
GTTATCCTCTAGGAGTTTTATAGTTTTGAGTTTTACATTTAATTCTCTGGTTTATTTTGAGTTT
ATTTTTGTGAAGGGTGTAAGACCTGTGTCTAGACTCTTTTTTTTTTTGCATGTGGATGTTCAGT
TATTACCACTGTTTATTGAAAAAGACTATTTTCTTCATTGTATTGCCTTTGCCTCTTTGTCAAA
TACTGCCATTGCTCCTTTGACTATGTGGGTCCATTTCTGGGCTCTCTATTCTGTTTCGTTGGTC
TGTTTTGTCAGTTCTTTTGTCAGTACCACACCATCTTGACTACTGTAGCTTTACAGTAAATCTT
GAAATCAGTAGTGTCCATCTGACTTTTTCTGTCAATATTGAGTTGTCTCTCACGGGTCATGGAA
TAGCTCTTCATTATTTAGTTATTTGATTTCTTTCATCAGAGTTCTAAAACATTTTTCCTCATAT
ATCTTGTACTAATTTTGTTAGATCTGTACTTAAATATTTCATTTTGGGACACGCTAATGTAAAA
TGGTATTGTGTTTTTAATTTCAAATTCAACTTATGCATTGCTGGTTTACAGGAAAGCTATTAAC
TTTTTTATATGAACCTTGTATCCTGCAGCTTTGCTATAATCACTTATTAGTTCTAGGAGGTTTT
TGTTGTTTTTCTTTTTCTTTTTTTCAATTCCTGTCTATGTTCTACACATATAGTCATTTCATCT
GCAAACAAAGGCAGTTGTATTTTTTTGTTCCCCGTTAGTATACCCTTTATTTCCTTTTCTTGTC
TTAGCTAGGACTTCCAGTATGGAGTGGTGAGGTGGGGATATTCTTTTCTTGTTCCTGATCTTAG
TAAGAAAGTTTCTAGTTTCTCATCATTAAGGATGGTGTTAGCTGTATGTTTTTTGTAAACGTTC
TTTATCAAGTTGAAGGTGTTCCCTCTATTCCTAGTTTACTAGTTTCTATCATGAGTGGGTGTTA
AATTTTGTCACATACTTCTCTCCATCTATGGTATGGTCATGTGATTTTTCTTCTTTAGTCTGTT
GATATGGTGAATTGCATTAATTGATTTTCCAATATTGAACCAGTCTTGCATACCTGGGATAAAT
CCTACTTTGTGCTGGTGTATACTTCTTTTTATACATTGTAGTAAAATTTAGTTTGCTAATACTT
TGCTAATACAAAGATTTTTGCATCTTTGTTCATGACAAATATGGGTCTGTAGTTTTCTTTTCTG
ATAATGTCTTTGTCTGGTTTGGGTATTAGAGTAATGCTGACTTCATACAATGAGATAGGAGCAT
CTCCTTTGCTTCTGTCTTCTGAAGGAGATTGTAGAGAACTGGTGTGGTTTCTTCCTTAAATGTT
TAGTAGAATTCATCAGAGAATCCATCTGGGCCTGATGCCTTCTCTTTTAGAACATTATTAATTA
TGGATTTAATTTCTTTAATATATGCTTTTTAATCAAGATTATTTCTTCTTAGAGTTCTAATAGA
TTGTGTCTTTTAAGGAATTGATTCATTTCATCTAGGTTACCAAATTCGTGGCCGTAAAGTTGTT
CATAATATCCCTTGATTATCCTTTTAATGTCCATGAGATCTGTAATGATGTCCACTCTTTTCAT
TCTGATATTAGTTATTTTGTCCTTTGTCTCTTTTTCTTAGCCTGGCTAGAGGCTTATTGATTTT
ATTGATCTTTTCAGAGAACTAGTTGATTTCTTTGATTTTTCTCTATTGATATCTTGTTTTCAAT
TTCATTGATTTCTGCTCTAATTTTTATTACTTCTATTTGGATTTAATTTGCTTTTTCTAGTTTC
ATAAGTTGGTAGCTCAGATAATTGATTTTTAGATCATTCTTTTCTTTTCTTTTTTTTTTGAGAT
GGAGTCTCGCTTTGTCGCCCAGGCTGGAGTGCAGTGGCTCGATCTCGGCTCACCGCAAGCTCCG
CCTCCCGGGTTCACGCCATTCTCTTGCCTCAGCCTCCCAAGTAGCTGGGACTACAGGCGCCTGC
CACCACGTGTGGCTAATTTTTTTGTATTTTTAGTAGAGACGGGGTTTCACTGTGTTAGCCAGGA
TGGTCTTGATCTCCTGACCATGTGATCCACCCGCCTCAGCCTCCCAAAGTGCTGGGATTACAGG
CGTGAGCCACAGCGCCCGGCCTAGATAATTCTTTTCTAATAAATGCATCAGTGTTGTATGTTTC
TTTCTAAGCGCTGCTTTCACTGTATCCCACAAATTTGGATAAGTTGTGTTTTCATGATATTTTC
AATTTATAATGGTATTGGGACACAACCCAATAAGTAAGTTTAGGAGCATTTGTATGCATAAGCA
CAATTGTTGACATTATTTTGAACAAATTCTTATTTGTTAGATCAATTAAGTATAAGAAAAATAA
AAGGTTTTATTTTACTTTCAGTGTTTCCTTCTTTTGATGCTCTATATATGTAGATTTCAGTTTC
TGAACTTCCCAACACTTATTGCAAGGCAGGTGTACTGACAACAAATTTCCTTAATTTTTGTCTG
AGAAAGTGTTTCTTTTTAACTTTTGAATAATAATCTCACAAGGTAGAGTGCTAGGTTTATGGTT
TTTTTTTCTCTCAACACTATGAATATTTCACTCTTTTTCTTGTTGGAAGTCACATGTATTTTTT
CTTTTTTTTCTTTTTTTCCTCTACAGGTAAGGTGATTTTTTCCCTCTGGCTTACTTCGGATTTT
TTTCTTATCCTTGAATTTCTGTAATTACAACGTTTTATGCCCAGATGTAGTTTTTTGGCATATT
TTCAAGCTCAGAGATTCTTCATCCATGCCCAGCCTACTAATAAGCCCATCAAAGGCATTCTTCA
TTTCTCTTACAGTGTTCTGTTTTTTGTTTTTGTTTGTGTTTTATCTCTAGCACTTCCTTTTGGT
TCTTAGGATTTTCATCTCTACTTTTTTAACATGTTGTTTACTCAGTTCGTTAGGGCCCTTAGCA
TATTAATCATAGTTGTTTTAGATTCCAAGTTTGATAATTCCAAATCCCTGCTGTATCTGGTCCT
GGTGCTTGTTTTTTACCTTTTATTATGCCCTGTAATTTTTTTCTTGATAGCTTGGACATGATGT
ACTAGGTAAAAGGAACTGTTGTAAATGAGCATTTAGTAATGTGGTTGTAAGGTGTAGGGAGAGG
AGAAGAGTTCTGTAGTCTTATGATTAGGTCAGTCTTTTTAGTGAGCTTATGCCTCTGGACTGTG
AACTTCACAAGTGTTTCTCAATCCCACCCCTCCCTCATTCCCTTAGTTAGAACAAGATAGCTAT
AATGGCCTGAAGTTGGGTTATTTCTCTTCCCCCACATCATTTTGGCTTTGCTACAACCCCAGCA
GGTTAGGCTCTGGTTAACTAGTTTCTCCTGAGGACAAACCTTGTTAAGACTGGAATAGTCTAGT
GTACTTGAAAATCATTTCTCTTTGCCTCCCCTGCTGAGGGTTGAGGATTTTTCTCCTATATTTA
CTGTGAGAAGCTGGTCAATTGCCTGGATGTAAAACTTAGAAAATTGTAGGGCTCCCCCTATAAT
TGGGTAACTCTGGAGTTTTTAGCTTTCAGAGTTGTCCACACTGAGCCTTCATCAATTTGTCTAT
TACACTTCAGGATTTCCTACCCCGTGGAGGTTTCTGCTTGTGTTTTTCATCTCAGATAAATGGT
GATGCTATGTGTTCACCTGTTCTCTGCTCTCACAGGCAGTCATTTGCCCTGTGACCTCACTTAT
CCTATCTCAGATTTGTTTATTTTTAAGTTTGTCATTTTTTACTTGTTGTTAGGATGGAGTGGTG
ACTTCCAAGCTCTTCATATGTGGAACTGGAGGTAATTCTATTTCAAATTTTTTGAGGGACCTTC
ATGTTGTTTTCCACAGTGCCTGGCACCATTTTACATTTTCACCAGTGGTGCATAAGGGTTCCAG
TTTCTCCATATCTTCACCAATACTTATTTTCTGTTTTTTTAATGGTGGCCATCCTAATGGTTTC
AGATGATGTATCATTGCAGTTTTAATTGACATTTCTCCCTACTGATCAGTGATGGTGAGAATCT
TTTCATGGGCTTATTGCCCATTTGTATATATTCTTTGGAAAAATGACTATTCAAGTCCTTTGTC
CACTTTTTAACTAGATTTTTTGTTGTTGAATTGTAGGAGTTATTTATATATTCTGGATATTAAC
CAAATAACAGATACATGGTATAGAAATATTTTCTCCTGTTTTGTAGGTTGCCATTTTACTCCAT
TTACTGTTTGCTTTGTAGAAATTTTTGAGTTTATGTATCCCCAGTTTGTCTATTTTTGCTTTGG
TTGCTTGTGCTTTTGGTGATCTATTCAAGAAATCATTGCCAAGTCCAATGTCATGAAGCTTTTT
CTCCATGTTTTCTTGTAGTAGTTTTATAATTCGATGTTTTAAGTTTAGGTCTGTAATCCAGCTT
GAGTTCATCTTTGTATATGGTGTGAGATAAGGCTCCAACTTCATTCTTTTGCATGTTAGATTCC
AGATTTCCCAATACAGTTTGTTGAAGAACCTCTTTTCCCTATTTCTGTGTTCTCTCTTCTGCTC
ACTTGGTCTGTATATCTGTCTTTATGCCAGCACCATAGTGTTTTGATTACTATAGCTTTGCAAT
ATCTTTAGAAATCAGGGAATGTGAGGCCTCTAGCTTTGTTTTTTGTCCAGATTGTTACGGCTAT
TCAGGGTCCTTCAAGATTCTGTATTAATTTTAAGATTTTTTTTTTCTGTTTCTGTAGAAAATGC
CATTGGGATTTTGATAGGGATTGCTTTGAATTTGCAGATTGCTTTGGGTAGTATTGCCATCTTA
ACAGTACTAAATCTTCCAATTCATAAACTTGGATGTCTTTCCATTTATTTGTATTGTCTTTAAT
TTCTTTCAGCATTGTTGTATAATTTTCTTTTTCTTTTTTTTTTTTTTTTGAGACAGAGTCTCAC
TCTGTCACCAGGCTGGAGTAGAGTGGCGTGATCTTGGCTCACTGCACCTCTGCCTCCCAAATTC
AAGTGACAACTCCTGCCTCAGCCTCCCGAGTGGCTGGGATTACAGGCGCGCGCCACCAAGCCCA
GCTAATTTATTTTGTATTTTTAGTAGAGACAGGGTTTCACCATGTTGGCCAGGATGGTCTCAAT
CTCTTGACCTCGTGATCAGCCCACCTTGGCCTCCCAAACTGCTGGGGTTACAGGCGTGAGCCAC
CACGTCCGGCCTATGTTGTTTAATTTTCAATGTACATGTCTTTCATTTGCTTGGTGAAAATATT
CCCTAAGTATTTTTTACTCTTTGATTTCATTTTAAATGGGTTTAAAAAATTAATTTCCTTTTTG
GATTCTTCATTTTTAGTATTTAGAAATGCAACTCTTTTCTTAATATTGGTTTTTATATCCTGTA
ACTTTACTGAATTCCTTTATTCTAACGGTTTTTTTTCTTTTTGGTAGGCTAGAGTTTTCAACAT
AAAAGATCATGTCATTTGCTAACAGAAGTAATTTTACTTCTTACTTTTCTCATTTAATACCTTT
CATTTCTTTTTCTTGCCCAGTTGCTAACTCTGGCAGGATGTCTAACAGTATGTTGAATAGAAGT
GGTGAATGTGTGCATTCTTGTTCCATTTGTGATCTTAGAGGGAAAACTTTCAGTTTTTCATCAT
TGAATAACATTAGCTGTGGTCTTTCTATATATGGCCTTCATTATGTTGAAGTAATTTCCATATC
TTTCTAGCGAATTGAGCGTTTTTGTTTTTGTTTTTTTGAGACAGAATCTTGCTCTTGTCACCCA
GGCTGGAGTGCAATGATGCTATCTCACCTCACTGCCAGCTCCGCCTCCCGGGTTCTCAAGTGAT
TGTCCTGCCTCAGCCTCCCGAGTAGCTGGAATTACAGGCGCCCACCACCATGCCCGATTAGTTT
TTGTATTTTTAGTAGAGACAGGGTTTCACTGTGTTGGCCAGGCTGGTCTCACACTCCTGACCTC
AGGTGATCCACCCACCTTGCCCTTCTAAAGTGCTGGGATTACAGGCATGAGCCACCCTGTCTGG
CCTTTTTTTAAATCCTAAAAGGGTGTTGATTTTTGTCAAATGCTTCTTCTGAGTCAGTTGAGAT
TATTGGTTTTTGTCCTAAGAATGCAGTGTATTAAAGTGATTTTTTTGTATGTTGAACCATCCTT
GGATTTTATGAATAAATTCCAGTTGAGAATTTATGAAATATAATCTTTTTAATGCATTGTTGAA
TTCTGTTTCTTGGTATTTTGTTGATTTTTTTTGCATCAGTATTTATTAGGGATTCTTTTTTCTT
GTAAATTTGTCTGATTTCGATATCTAAGTAATGCTTACCTCATAAAATGATTTTGAAAGTGTTC
TGTCCTCTAGTTTTTTCAAAGAGTTTGAGAAGGGTTGTTAGTTTTTAAATGTTTGGTAGAGTTT
TCAGTGAAGCCCTGGCGTTGTTGGACAGTTACTGATTCAATCTCTAATAGTTATAGGTCCATTC
AGAAGTTGTATGTATTTGTGGCTCAGTCTTGGTGGGTTTGATGTGTCTATCCGTTCTCAAAGGT
TATCAAATTTTTTTGGGTCATAGTTGTTCATATTAGTCTTTTAAGGTCCTATTTATTTCTGTGG
CAGCAGTTGTTGTATCGCCTTTCTAATTTCCAGTTGTTGCTGTTTGTGTCTATTCTCTTCATTT
CTTAATCTAAGAATTTGCCAGTTTTGTTGATCTTTTCAATAAAACAGCTACTCCTAATTTTGTT
GATTTTTTTTCTATTGCAAAAATTTTCTATTTTTCTCTATTTTATTTTAGCTGTAATTTATTTT
TATTTTTTTTATTTTTTGGTAAGTTTGAGTCTCATTTGTTCTTCATTTCCTAATTCCATAAGAT
GTAAAGTTAATTTGTTGATTTAAGGTTTTTCTTCCTTTCTAACTGTGAAATATGACACTGTAAA
ACAGATTTTCATTTTACAGATGAATTATGTAAATATTTAGTTCTACAATTTTACTTTTTTTTTT
ATCATATATCACGCATTCTGGATATTGAGACTATAAAAATGAAAAAAACAGTTCTTTTCTTTTC
TTTTTTTTTTTTGAGACAGAGTCTCGCTCTTTTGCCCAGGCCACAGTGCAGTGGCTCTGTCTCG
GCTCACTGCAAGCTCCGCCTCCCGGGTTCATGCCATTCTTCTGCATCAGCCTCCCAAGTAGCTG
GGACTATAGGCGCCCACCACGGCGCCCAGCTAATTTTTTTTTGTATTTTTAGTAGGGACGTAGT
TTCACCATGTTAGCCAGGATGGTCTCGATCTCCTGACCTCATGATCCGCCTGCCTCGGCCTCCC
AAAGTGCTGGGATTAGAGGCGTGGCGTGAGCCACTGCACCCGGCCATAAAACAGTAATTGAAGG
TTAACCTATATTTCAAACAACTGAAAATACTGATTTGTTCATATTTATTTTTTCATGAAAATCG
TCTTGTTCTATATTTAATAGGTTTTTGTTCGGTTCATGGTACTAATGAGTTCACAATTGTAGGT
TTAGTCTTGAAATAATCTAAATTCACTTCATCCCATTGTAACAGACCCTACATCATACTGAATA
TAGTTGTGTCACAGATGTCTCTTACAGTCTATTTCTACAAGGCCTAGAACTATTTAGGATACCG
TTGCTCACTCTTACAAATTTTAACATTAAAAATAACTTTGAGATGGAATGGAGTTTTGCAACGT
CTCAGAACAAAGAGACTCCACTTACGAGAAATTACTAAGCAGTGGTTCCTAGCTTTATAGACTA
ATTATATAAGCCTTCAGTTGTTTCTATTTTATATCTGTTTTTTAGATTCCTCACATGTTCATTC
TGCTTTACCTTCTGTATAATATAAAGGTCACTCTACTTACAGTCACATTTTCCTTTTAATCTTT
CCGTTTTTATTTTTTATTCTTTGCTTTGTTCACCATTAGTTTTTATATTGCATCTCACAGTTCA
TGTTTCTTTTTTGTCATTTCTGATTCTAATTGTAATTCTAATCTTACTTGTTTGGCGTTCACTG
TCACATCTTCACTTTCTGGCAGCTGTTTCTATGGCTTCATCTTCTTAGTGCTTGTATTCATTGC
TTATATTCCAGTTGTCTTGTAAATTCATTTTTTTAGTGAACTAAGCCAGACACGGAAAGACAGA
TATCACATGTTCTCACTGATATGTGGGAACTAAAAAAACATTGAACACAAATTTTACAGCTGAC
ACACATATGCCTGCCACCTAGATTCTGCCATTAATACTTACATTGCTTTATCGTATAACCATCC
TTGTATCCATCAATCCATCTTAATTTTTATGCCTTTCAAAGTTAATTGCTAACATTAGTACACA
TCCCTCTAGGTGAAACCATTTTTAAACTGATTGCTGTCAAGTTGTCTAGAGTAAACTCTGATCT
TCCAAGTATGACTTATTCCATAGGACTTCTGTTTGTAGCACTGTCCCATTAATTCAATCATAGA
AATAAATAATTGGAAAGTATTGTCTGTTTTAAATGGATAAGTATTTTCATGTGTCTGACTTTAG
AGGACTTTTACTAGAAGGTCTCACTTGACCTTCATTGCTGACTAAATAAGAAAAGTTTGTTTTA
TTTTAAAAATCAACACATTATAACATACTTTGTTTTTTGAGTAATAAGAAAAGTCAATGGAATG
TGAAGAAATTATAGCAAGTAAGCTGTTGTTCCTCTTCTAGGAATTTTGATTATGAACAGCTGAT
AAGGTATTACTAAGGGATCTCATCAGGGAAATTCCTTTCTTTCCTTCTTTTTCTTTTGCATCCT
CTCCCTTTCCTCACTTTCTTTTCCCCTTTTTCCCTTTTCCCCTCTCCTCTTCTTTTCTTTAATA
GTACTTGGGGAAAGGTATACCTTTTATTTTACGGATTTGCTCAGGGTAAAGAGAGAGTTAAGAG
GCCAAATGATTAGATGGCAGGGTCACTGGAGGGCAGAGATAAATAATAGATCCTAAACTAAGGG
TTGGCAAACATTTTCTGTAAAGGGCAGGTGGTATTTGTTTTAGGTTTTGCAGGCCTAAAGTACT
CAACTCTGCCATTGTAGTATGAAAAACAGCCATAAACTATATAGTATATTGTATTAGTCCTTTT
TCACGCTGCTGATAAAGACGTACCTGAGACTGGGAAAAAAAAAAGAAGTTTAATTGGACTTACA
CTTCCACATGGCTGGGAAGGCTTCAGAATCATGGCGAAAGGTGAAAGACACTTCTTGCATGGCA
GTGACAAGAGAAAATGAGAAGGATGCAAAAGCGGAAACCCCTGATAAAACCATCAGATCTCGTG
AGACTTACTACCACAAGAACAGCATGTGGGAAACTGCCCCCATGATTCAGATTATCTCCCAGGG
GGTCACTCCTACAACACATGAGAATTATGGGAGTACAATTCAAGATGAGATTTGGGTGGGGACA
CAGAGCCAAAGCATTATCATATATAGTATATTTATAAATCAATGAACTTGGGTATGTTCCAGTA
CACTTGATGGATACTGGAACTTGAATTTCTTATAATTTTCACGTTACAAACTATTATTATTTTT
CAAACTTTTTCCAGCCATTTAAAAATGTAAAATATTCTAAGCTTGAGAGACATAAACAGGTGGT
GGACTCGATTTGACTTACGAGCTATGATTTGGTGACCCATATGGTACAAGGATTTCCCTTGCGC
AGGAACTATACCAAAAAGTCGTTATTAGTGTATACTTTTCCAACGTAGAGGCCCTTTTATGTGC
TCTAAATTTTGTATGCCTTTTACATATCCTAAATTAGATAGTGATGCAGATGAGGTCAGATAAA
TTAAGGTGTCATTGGAGTGAGGAAAAAAGAAGAATGCTTAATATAGTTGATTCCACCATTAACT
TGGAGGTTCTGGCATGGCTTCCTTTAGTCCTGTCTCATCAGTGTAGCTGCTATTTGTTAAACTT
GGCACAGAAAGGCAGATCAAATATAGAGGGCTACTTTCAGATCCTCCTCTTAATAGTTATTAAA
AGCTCAAGTTTAGTAGCTGAGTTCAAATCTCTTATTAGATGTATGACTTATGTAATTTCTCTGA
GCATCAGCAGAATGAAGGTTTATAGTGCTAACTCATACACTTGTGAGGGTGATAGTAACAATTA
CAACAATAATAGTAGTAGCTAACACTTTCATAGTGCTTAGTATGTGCCAGGCAATGTTCTAAGC
ACTTTATATATATTAACACATTTAATTTTCTCACAATTGGTAATGTGAAGAAGAATAATACTCT
CACCATGATAATGCTCGTAGATACTACCATTTAAATTACGAGGAAACTTACGTCCAGAAAGATC
AAGTAATTCATTCAGGGTCACATAGTTAATTAAGAGGTAGAGCCATTATGTGAACTTGGGCATC
TGGCCTCAGAATGCGAGCTCTTAACTGTTAAGGTTTATTCCCCTCTAAAGTGGGAATGTCTATA
AAATGCATAGCACATAGGTCCTCAACAAATTAGAGAGAAATTAACTTTGCAGAATATAGTGGTT
CCATTTCCTCAAGAAACTTGGTCTGCCTTTGTGGTACCAGATAGATGCATAAATGTGAGGTTCA
CGTTATGATTCTTCAGATTTTTTTCCCAGTTGCACATTAGTTATGAGATTTTTTTCCATAGAAT
TCTTCAGTATTACTCATGTTAAAAAATAATTATTTTGAAATGAATCACCTTCTGCAAAACAATA
TAGTCAAAATGACTTAGACTCTGAAGCTGAAAGACTCCTGGACTCCTATTCTTGTCTTGGTTCT
GTACTTTCTTTGAGATCTTGGTTTCGTGATTTGACTCCAGTTCTATTTCTCTCTTTGGCTGTGG
ACTGAAGATAATCCTCAATGTCATGGTATTCAAATGAGATAACCCATGTCAAAGTGCCCCGCAG
TGTGTGTAATAAGTTATCAGGCATCCAGCAAATACTAGTTTCTTGCTCACCTCCCTATTACATA
AAATAACTTTAAAATTAGAACTGAAGGGACTCATAAAGCTGGGAAAACTGAGGCCCAGACAATT
GTTTCCCTAGGATCCTACAGCTGCTTAGGGACAGAAGGATGACACTTAATGCAGATCTTCTGAT
TTTTCAGTCTCGTTCTGTTTGTGCTGATCACTCTCCTCCTATGTAGTATATAAAGAAGTACTAT
GTATCTGACAAGTTTTGGGGAGGTTATTTTAGTATAAAAGCTCACATTAAAAATATCTATTTTG
GTATTTTGACTTTTGTAATTTTTTTAAAAATCAGAGACAATTTAGTAAATCTCGGGGCCATTCA
AATCATGTATATGAAGTTTTATAACTGCTATTGTTTTCCCCTTATAGTTATTAATTTTTAATAT
GCAAGTAGCATCCTTACTATGGAAAGAATAAATAAGAAATCAAAAGATGGAAGAAGTTTTAAAA
CTCACTTAATCACATCATCCAGAGAAAATGCTATTAATGCTTTGAAATAACAATGGGTTCATCC
TACACCTGCTACTTTTTAACTGACTTTTGTCAGTTAGTGATATATCAGGAAGACTAGGCAGTTC
TTTGTCGAATTCAAATCTTGATAGTTTTTATTTTATTGTGTGACCTATTATCAGTTCATTTAAG
TAATCTTTTATTAGACATGTTTTTTTCAGATATAGAAACAATGCATTTTTGTAAAATTGTCAAA
TTATTTTCCACAAGTAGGATCTCATTCACACGGTATTCGTGTTTTTAAGACTTTCGATACATGT
TCACTGTTTTCCTCCAGAAAGGAAATACCTACTTACATTTTCACCAGGGAAGTCACTGTAGTTT
GGGAGAACATGACATTATCTCCCTTTCTTTACTGAAGGGTAGCAATATATATTTTAAATTTTCT
AGATTCTGCATTTCCATTTTATTTTTACATGTTTTCACTTTCATATTTTGCATAGTGCTAAATC
TGTGATAACAGTTTTTAATTTTCAGGTGGGGTTTATAGCTTCATAGATAGCAAAATGTCTTTAA
GTCATGTTTATGCAATTTTTACATGTGCTTTGTGGGTTTTCTTTTTTCTCTGTTTGCATGCAGT
GAGTGGTAAATTTGCATGGAACATTTGAAAAATAAGCACCAGAATGAATTGAATGTGTGTGAAT
TATCACTTTTTGTCACCTACCTGGACAGAGAGTGAAAGTTCTTCTGTTGAGACAAAATTGGATC
TCGTGTGTTTCTTCACTTACTTGCTGCCTTGTTAATTGTTGAGTTTCTCATGATTAGCAAGATG
TAAACTCTGGCATTGGTCATCTAGTCCTACATGTTAGATAATTCTGTGAATGTTTTGAAAATCA
GCTGAACTATTTTTCTTATAATTTTGATTCTTTGTAGTTGTTATTTCCATGCTTGTGAATTTGG
GACCAAAAGGAATGGCTATGACTTATGTTTCTTTGGATCCAGTAGTTTATTCTACTGAAATTAT
GGTAATTTTATCAGTTAGTAGAAGCATAGACTCTTTAGTGTTGAATATAATGTCATCTAACTTC
TTACATGGTCACATAATTCCTCCCTCAGTTTTGGTAGCTCTCCTGAAACAGCCATAATAGGACA
TTAATCTGTGTACTTTAAGTTGACAGAGTAAAAGGAATATAAGATTTCACGTGATGGAACTTAA
AATATTCCATCTTGTGACGTGGAACTAATGAGGAGGTTTAGTGACAAGTTTTAGATTTAAGATT
CTACACTTTGGAGCCTCTTTTTCTTTTTCTTTTTGGTTTTTGTTTGTTTGTTTGTTTTTGAGAC
AAAGTCTAGCTCTGTTGCCCAGGCTGGAGTGCAGTGGTGCAATTTCAGCTTATGGCAGCCTTGA
CTCTCAGGCTCAAGTGATCCTCAGCCTCCCAAGTAGCTGGGACTACAGGTGCATGCCACCACAC
TTGGCTAATTTTTTTTAAAAAAGTTTGTAGAGAGTAGCCTCACTATGTTGCCCAGGCTGGTGTT
GACCTCCTGGGCTCAAGTAGTCGGCCTGCCTCAGCCTCCCACAGTTATGGGATTGATTACAGTC
ATGATCTGCCTCACTCAGGCTGCAGCCGCTTTCTTTCAGTGTAACACTATCTTTGGATATTCCA
CTTACAAATTTATTTTTAAGTCTCCCATGTTGTTGATAATTGGGAAAAATAGTTTTTATTCCAG
ATAGATATTCTGTGTTAACTATAAGTCAAATGTTTACAAGCTGTTAAAAATGAAATACTGATTA
TGTAAAAGAAAACCGGATTGATGCTTTAAATAGACTCATTTTCCTAATGCTAATTTTTAAAATG
ATAGAATCCTACAACTCTTAGCTGTAAACCTTGTGATTTTTCAGCTGTTGTACTAAACAACTTA
AGCACATATACCATCAGACAAGCCCCCCTCCCCCCTTTTAAACCAAAGGAATGTATACTCTGTT
AATACAGTCAGTAAGCATTGACATTCTTTATCATAATATCCTAGAAAATATTTATTAACTATTT
CACTAGTCAGGAGTTGTGGTAAATAGTGCATCTCCATTTTCTACTTCTCATCTTCATACACAGG
TTAATCACTTCAGTGCTTGACTAACTTTTGCCTTGATGATATGTTGAGCTTTGTACTTGAGAGC
TGTACTAATCACTGTGCTTATTGTTTGAATGTTTGGTACAGGAAGCGAGCAGCTGCAAAGCATC
TAATAGAACGCTACTACCACCAGTTAACTGAGGGCTGTGGAAATGAAGCCTGCACGAATGAGTT
TTGTGCTTCCTGTCCAACTTTTCTTCGTATGGATAATAATGCAGCAGCTATTAAAGCCCTCGAG
CTTTATAAGATTAATGCAAAACTCTGTGATCCTCATCCCTCCAAGAAAGGAGCAAGCTCAGCTT
ACCTTGAGAACTCGAAAGGTGCCCCCAACAACTCCTGCTCTGAGATAAAAATGAACAAGAAAGG
CGCTAGAATTGATTTTAAAGGTAAGATGTTTTATTTTCAATTGAGAATTGTTGCCTGAAAACCA
TGTGGGAGATTTAAATGTATTAGTTTTTATTTGTTTTTTCTTCTGTGACATAAAGACATTTTGA
TATCGTAGAACCAATTTTTTATTGTGGTAACGGACAGGAATAATAACTACATTTTACAGGTCTA
ATCATTGCTAATTAGAAGCAGATCATATGCCAAAAGTTCATTTGTTAATAGATTGATTTGAACT
TTTTAAAATTCTTAGGAAAAATGTATTAAGTGGTAGTGAATCTCCAAAACTAGGCCACAACATC
TATTTAAGTTATAGAATAACTGGACTAAGTATCTGGCATGAGTTCTGGAACCTCAGGATGATGT
GATGAAAGAAGCAGAAAGAGGAAACAGTGGGTTTTTTCCATTTTTGAAGGCAAGACAAAATTTT
GTGATAGTTATGTGAGAATTCTACGTTTCCTTCAGATATATGCTTCTCACTCTCCTTCCGGACA
TTTGGTACAGTCATGCTGCCAAGGATCATGACCTACCAATGACAGTCTTCACTCAGGTCTTTTG
TTTTCTAAATGGTCCTACGTCCAAAATTCAGAATTCGATTACCTTGTCGCTAATACTAAAACTC
CAAGTTATTTTCTTCAGTGAAGGTAATTAGTAACAAAGTAATCAAATTTTTGAATGACTACCAG
GGCTTAGCGCCCCTCCACTCTCACCCCTACACACACCCAGAGGCAATTGTATTTTAAGAGAGAA
AGCTTTTTTTTTATGACATACAAGTCAGAGAATTTTAGAGGCATTTGCCTCTAAAACTTTCACT
AACATCATTTGAATCAAGTTTGAAAGAATTAGTGAAGTGGCATTGTGTTATAACCTCTACTCTG
TTTCCACAATAAAATTATTTTCGTCACTTTACACAACTTTGTTGCTTCAGAGTGTATAATTCTG
TTTCATGAATTAGTTCACACACTATGGGAAAATAGGTAAATGTTGTCTGTATATATAGTGTGTC
AGTATAATGTGGAAGCTGCATTGACTTTATTGTACACAGTTCTCCAGTAAGTGTGCACCAGAGA
GTACCAGTAACTGAATGTAGAGTGCTGGCACAGTGGAATGCAGCCAGCTGCGGGGGATGTGGTA
CATGAGGAATGTGCTCATCTGCTCTTTTGGCCACGTGCTCCATCTTAGAGGTGCTTATTGTGTG
GCTTTATCCAGTCTTTATGCCCCCTCCTCTTTTTGCTCAAGTCTGCATTATAGCAGTTGTGACC
TTTCCTTATCCCTGTTTTCTAATATGCTACTGTTACTGTTCCCTGTTCGTTACTGGTTTTTTTG
TTTTTGTTTTTGCATTGTTCTATAGTTACTATTGTATATTGGAAATTAGAGATGCTTTCTGTAC
ACTAGTACTCTTATTTTGATTATTAATTATGTTTGTTAACACTAATTACAAAGTTGCATAAGTA
TTAAGTAGTCTGCACCAAACTGTTTTTGCGTAGGAGGTTTTTAAGTGCAAAATTTAAAAGATTG
AGTATTTTCAGGGACACATTTATCACAAGAAATCTTATAAAAATGTCTTTCTCAGTAGAACACC
TGTATCAATAAACCAAACTTTTTGAATGTTCATATCTAATTACAGTGTTTTTTGACTAATTTTT
ATCTATATACCTGAAAAATTCAGTTTAACATATTAAACACTACAGGCCAGGCACAGTGGCTCAC
GCCTGTAATCCCAGCACTTTGGGAGGCTGAGGCGGGCGAATCACGACGTCAAGAGATCGAGACC
AACCTGGCCAACGTGGTGAAACCCCCTCTCTACTAAAAATACAAAAAAATTAGCCAGTTGTGGT
GGCGTGCATCTGTAGTCCCAGCTACTTGGGAGGCTGAGGTAGGAGAATCGTTTGAACCCGGGAG
GCAGAAGTTGCAGTGAGCCGAAATGGTGCCACTGCATTCCAACCTGGCAACAGAGTGAGACTCC
ATGTCAAAAAACAACAACAAAAAACACTCCTTGGAAAAAGTATAGGTACATTCTATATAGGATT
AGTAGACTGTCAGAATCACTTGAAGATGTTCCGAAAGCATGACATATCAACAAAGGACATGTGA
GTCTAGAATGGTGATCCCTAGGACAGGCCATATTACTGCATGCAAAGCAAGTCTTTGTTAACCT
TATGACTTGCTCAGGAAGCAAAGTGTTTCATGTCCTAATAGGATAGTAGTATTTTCTGTTATGT
TACATTTTAGGAAATTAGGAGTTTTGCATTGTCTTGTAAGAAGGTATTTTAAAAATTTTCAATG
AAAATAATGAGAAATAGGTTTCCAGTGGTTTTATGCTTTTGGGGAAGAGGTTACTGATGTTTAG
CAAAAGATGCTTATATATTCTTAATAGAACAAACCTGAGAATTAGTTCTTAAAGAGTTAAGGCC
CATAAAAGAAACTGTAAGTGTTGGTACTCTTGTAGCTGGGCCACAGATCAGATCCTTAGTATGA
TAAAATTTGGTATTTTTGTGCTTCAATTTTTGGAGACCATTCAATGATCACTTACATTTTATAT
TGTCTTTCATTGTGAAACATATATTATAGCAGAGGTGGGAGTCATTAAAAATTCTACATGTTGT
ACACTACATGTATAAGGGTATATTTGGCGCAATGGAGGTAGGATTGTCTGTGTGCCTCTAAAAT
TAACCTCTCAAGTAGTTAATCTGATGATGACAAGTATGTCACTTTATTGATAAAAGGGAGAATT
AAGACTATTCTCAGTATTATGTTGGTCAGTACACAAAATATATCATTTGTTTTTACCTGTGAAA
TGAAATATCTTTCATCCTATTAGCCAGAAGGGAGAAAGCCTTGGCCTGAAAATAACACTTATCT
CAAAAAGTTGGGAAATGTGGTCCAACTCTGTGCCCAGGAAAAGAGGAAATAGATTTTGATGACT
ACATAGTCTCTGCACTGCTTGCCAGCTCATAAGTTTACTTCTTTGCACTGTTTGTTTTCATTGC
TCAAAAACTATTTTCTGTCACTCTATGAGATTTAGTCTAACCTGATTCCTGGCTGTGGAAAGCA
GTTTCTACTTAAGAGCCGATGAGAAATGGTATATTCTTCTCAATCACTCACAGAATGACTACCC
TGGAACAAACTAGGAAGACCCAAAATGAACCAGTTTGATTTTAATACATACATAGATGCTGAAG
GAGCCTTTTTTTAAGACCTTGCCTTCTTAGTCTTTGGAGAACAGAGTACTGAAACAAGAAAACT
CACAGATTAGTAGTATTTCTGTGTGTTCTACAGTGAGGTTTTAAGAGCTGTATTATGATTAATC
AGTACATGACATATTGGTTCATATTTATAATTAAAGCTATACATTAATAGATATCTTGATTATA
AAGAAAGTTTAAACTCATGATCTTATTAAGAGTTATACATTGTTGAAAGAATGTAAAAGCATGG
GTGAGGTCATTGGTATAGGTAGGTAGTTCATTGAAAAAAATAGGTAAGCATTAAATTTTGTTTG
CTGAATCTAAGTATTAGATACTTTAAGAGTTGTATATCATAAATGATATTGAGCCTAGAATGTT
TGGCTGTTTTACTTTTAGAACTTTTTGCAACAGAGTAAACATACATATTATGAAAATAAATGTT
CTCTTTTTTCCTCTGATTTTCTAGATGTGACTTACTTAACAGAAGAGAAGGTATATGAAATTCT
TGAATTATGTAGAGAAAGAGAGGATTATTCCCCTTTAATCCGTGTTATTGGAAGAGTTTTTTCT
AGTGCTGAGGCATTGGTACAGAGCTTCCGGAAAGTTAAACAACACACCAAGGAAGAACTGAAAT
CTCTTCAAGCAAAAGATGAAGACAAAGATGAAGATGAAAAGGAAAAAGCTGCATGTTCTGCTGC
TGCTATGGAAGAAGACTCAGAAGCATCTTCCTCAAGGATAGGTGATAGCTCACAGGGAGACAAC
AATTTGCAAAAATTAGGCCCTGATGATGTGTCTGTGGATATTGATGCCATTAGAAGGGTCTACA
CCAGATTGCTCTCTAATGAAAAAATTGAAACTGCCTTTCTCAATGCACTTGTATATTTGTCACC
TAACGTGGAATGTGACTTGACGTATCACAATGTATACTCTCGAGATCCTAATTATCTGAATTTG
TTCATTATCGTAATGGAGAATAGAAATCTCCACAGTCCTGAATATCTGGAAATGGCTTTGCCAT
TATTTTGCAAAGCGATGAGCAAGCTACCCCTTGCAGCCCAAGGAAAACTGATCAGACTGTGGTC
TAAATACAATGCAGACCAGATTCGGAGAATGATGGAGACATTTCAGCAACTTATTACTTATAAA
GTCATAAGCAATGAATTTAACAGTCGAAATCTAGTGAATGATGATGATGCCATTGTTGCTGCTT
CGAAGTGCTTGAAAATGGTTTACTATGCAAATGTAGTGGGAGGGGAAGTGGACACAAATCACAA
TGAAGAAGATGATGAAGAGCCCATCCCTGAGTCCAGCGAGCTGACACTTCAGGAACTTTTGGGA
GAAGAAAGAAGAAACAAGAAAGGTCCTCGAGTGGACCCCCTGGAAACTGAACTTGGTGTTAAAA
CCCTGGATTGTCGAAAACCACTTATCCCTTTTGAAGAGTTTATTAATGAACCACTGAATGAGGT
TCTAGAAATGGATAAAGATTATACTTTTTTCAAAGTAGAAACAGAGAACAAATTCTCTTTTATG
ACATGTCCCTTTATATTGAATGCTGTCACAAAGAATTTGGGATTATATTATGACAATAGAATTC
GCATGTACAGTGAACGAAGAATCACTGTTCTCTACAGCTTAGTTCAAGGACAGCAGTTGAATCC
ATATTTGAGACTCAAAGTTAGACGTGACCATATCATAGATGATGCACTTGTCCGGGTAAGTTGG
GCTGCTAGATTAAAAACCTAATAATGGGGATATCATGATACAGTTCAGTGAATTCATTTTAAAA
GTGACTGAAAAAAATGATACCATATAGCATAGGAACACATGGACATTTCTGATCTTATATAAGT
ATTATACTTTTGTTGTTCCTGTGCAAGTTTATAGATGTGTTCTACAAAGTATCGGTTGTATTAT
ATAATGGTCATGCTATCTTTGAAAAAGAATGGGTTTTCTAAATCTTGAAAACTAAATCCAAAGT
TTCTTTCATTCAGAAGAGAATAGAGTGTTGGACAAAGACCAGAACAAGAGAAATGTGGAGATAC
CCAATAATAAGTGTGGATGTGCAGTCTTGAACTGGGAGTAATGGTACAGTAAAACCATACCATA
AAATTATAGGTAGTGTCCAAAAAATTCCATCGTGTAAAATTCAGAGTTGCATTATTGTGGACTT
GATGAAGTGACCATGGCAGATCAGTGCCTGTTGCCAGTAAGAGTTGAGTCCTGCTGGGCCACTT
AGAATATCCCTGTGTGGACGGCACTGCCTGGCTGTGGTAGTTTCTTCCCACTTAAAAATTCATA
GAGACAACTCTGGGTCTTGGAACTTCTGGAAAACAGCAGCTATTCCAAAAATCTGGGGATTAGT
GGTGCCTGTAACATCAAGTCCAGGGAGATTGAGCAGGGAAATACTGGAAACTCAGGTACTCATC
CTAACTTCCTGGTTTTCCAGATGGGGTTGCCATTTGTTCCTTTTCTTTCCTCCACAAGATAATT
TTAGTGTATCTGTTTAAGAATATAAGAGTGGCCAGAAGACAGAAAGCAGTCGTCAGTCACATTT
CACCAAAATTTACATTTGTCTCAGGTGCATTATCGCAGAGCTTTATTGTATTTCTAGTATTAGC
TATCCCTGTCATTGCTGCTACTTTTTTATCATCTTCAAGTAAAATTTAATGTCAACAAAAATTA
AGGTAGTTAATAATACTATAGTATACTAGTAAAGTTGTCTGACACAGACAACTAGGAAATGTGA
TTTTTACTTTAAAATGTATTTTAGTTTTAGAATGTCTAAATTTTAGTTTTAGAATTGTCTAAAT
TCGAGAATTCATAAAGCTGTAGCCCGGTGGAAAAAGTTATTTGACATCTCAGAAATTAAAAATT
GCAGAGATTTTGTGTGTTTTTTTTTTTTTTGTTACTGGTTTGTTTTTTTTTTTTAATGTCAGAT
ATGTTTATAGTGTTGGAGAAAGATTCTGCATTATTTAAGCAACTCAGGTGGTTTTCTAGCAGAG
ACAAAAGGGGGCTTTAACTGCCTGAAGTCATTATTATTGCTTGCTTTCTGGTACCCTTTTTATT
TCACTTACATAGTTTTGAGACACATGAATAATTAATACACTTAATGACTGATCATAAGGCAGCC
AGCACCTTACTTTACATAGTTTAGCAGAATGATAGGGTATTTTATAAGAATGTGAGTGATGTTT
TATGAAAGGTATGTTGAGGTCTGTCTATGACTATCCATTGTTGGAGCAGAACTGCAAATGCCAA
AGCTACATACTTTATGTTTGGCTTCTTGAGGTGAGATCCCTTCAGCTGTCTTCATCAAAAGCTA
TGTCTAGCCTTTAAAAGGCAATGAAATTACACTGTAAGAATCATTCCAGGGTTCAAAAAATTGT
CTACCTTCCTTATCTGTCTTTCGCTCAGAAGTCTACTCAGTCTTATTTTCCTCAGTCCCCCATT
TTTTTCTCTAGTAAAGCAAGACTAAAAACAAAAGTTTAAGTACTTGGTATATACTTTATTCTAT
ATTTTAAATTCTATTCTAGAATTTTAAGGAAACAACTGCTTTTAAAGCCTTAGGTGTTAGTGTA
AAATTAACTGTGAGCTCTTTAATTATGGTAACTTGTTTTTAGCAAGTTCTGCTTAATTATATCA
AAATTAGATAATATAGTGTGGCCTATCATAAATCAGGATTCACTAGATAGTATATGTACTTGAA
TATTGCCAGCCCACAAAAGCAGTTCACTTCAGAAATCATGTGTGTTAGGCAGTTTAATAAACTG
TGAAAATAGAAAGAGATGATGCTTACAGAGAAGCTTCCTTAGGGGCTCTGAATGCTCACACTAA
GCAGATTTTACTCTTAACTTTCTTTCTCCCTTGCTTTTTCTTCCTTTTCTTTTTTTTAAAAAAA
TAACAGAATGTGAAAAGACTAGAAAAACCAGCATCGAAATAGGTCTTATAAGTAAAATTGATAA
CCTGTGTAATTTTTTGAAGTAAGTATTCACAGCAGGTGATGATTATGATAAAGCCACATATTTT
AGTAACACATAGTAGAAGTTCTATAATAGAAAGTGGTTTAAGATTATTCTCTCATATGAAAGCT
CAAAAGTGATGAATTTTTCAAGATCTTATTTCCCAAGGATCTCTGCTTTTTAAGCCTCATTCTT
ATATATCACAGTGAACAAACTAGTGGCCACTTTTTTATGACTTGCTAATTTTGGAATTGTATAG
CAGGGTTGGGTGGAAAAGAAGAAATGAGAAACTTTGTGGTAAGCTTAATATTGATAAATAAAAT
TATGGGATCCTAGGCTTTAAGAGCTAAAAGAAACCTTAGAGTTATCCACTCCTTAGTTTAGAAA
TCAAGCCCAGAAAATAGTGACTTCATATATGAGACTCTTGTATGTTTTCTGTTGAGGAGTGGTA
ACTTGAATTAATAATTGTGGATTTTTTTAAAAAAGAAAAATATATTCATTGCTTTTATATATTG
TGAAATTCAAACCAGACTAGCAGCTTTTTTCCTGAGGTTTTTAGAGAAATTGTCTTTGATGACA
AGAGAAGAATTCATGTTGATAGTATTTTCACTAATCTCTGATTGTAGGAAACACTATCATTTTA
TTACTGCTAAGACAGAAAAATACCAGCAATTCATTGTAAGATGCTATCATTTGTATGGTGTAAT
CTGATTTCAGAGATAATGTGAAAAAAGTGAATTTTAGAATCAATAGAATATAAGTTGTCTCTTC
ACAAATACCTTAGAACTGCTTAAGAACACCCCCACCCTACCCGTTTAACTTACTGCCATAGTTC
CCCTAGGACTTATTTTCAGTATTTTTTCGTTATTGTTCCTTAGAAAACAGTACAGTGATAAACA
TGAGCTTAGACTTCACCTTTCATTTGAATGACATTCCTAATGTGATGAGGTTTTTACAAATATG
TAAAACCATATTTACAAATTTACATTTTTAATATAAATATGTAATTTAATTTAATATGTAAATT
AATTTAATTTAAATATGTAAATATTTACATATTTACAAATATGCAAATATTTACATATTTACAA
ATATGTAAATATTTACATATTTACAAATATGTAAATATTTACATATTTACAAATATGTAAATAT
ACCCATTTGTGTGTTCTTTTACAAGTAGATAATTTCTCATTTTTCTTAGATGAAGCACTTCCAA
TGATAGTATGTATTAAGATTTTTTAAATTACTTCTTAAAGGAGAAGATAGAGGTTATAAGTTTA
TGGAAGAATATAGTATTGCCATCATGAGGTCCCTCAAACTGCCTTCACATCTGAATGGGAAAAT
TGTTTAAAAAAAATAAATAAATAAATAAATAAAATCCAGGGCCCTAACCTCTAGAGGTTTAGAT
TAGAAGCTGTATTGTAGAGCCTATGAATCTGAACTTCTGGTAATACTCCACAAATATTTTGATA
AAGCCAGACTCCAATATTTGGGAAGATGGCCTTTGGTTCAGCTCAGGTGGGGGTGTGTGTGCAT
GTGTGTTTATGTGTGTATGTGTGCATATATAGAGAGAAAGAGAAAGGCCATTTTGATATACAAG
TATGTTTATGTGTTTTGGAAACTCAGATTTTTAAGAGGATCATGGGGACAGCAAGCTTGGAATA
GAGGATGATGATGATGTGAAACTTGGTTCCAATTAGTCAGGGAGTTCTAGCATTTCACATAAAT
ATCTAAAAACCAAAAGGAATGGCTGAGATGTGGAATTGAATAGGGAAGTTCAAAAATAGGAATT
AGCACAGATAAAGGTGATTGGCACAGATAAAAAGCCCCACTACCAGGAGGTTCTGCAAAATCCC
CTCTAAAAGTCACTGGCCCTTTTTATCTGGTTTCTAATAGCCTAGTCTAACCCAGCTTCCAGCA
TTTTCAGCTCCCAGCTTCCAGCATTGGTGAACACTCTTACTAGTGAAACATGAACGTAATTCTT
TTATTTATTCATTGACTCACAGAGCTTTAAAAATGAGATTATTCTGTGCTGCACTAGGTATGTT
TAATTTTGTCTTACACATACACTATATTGTGGTTAACATGTAATTTCCATTGTTCTTTCTTGTG
ATTTATTCTAATTGAAATCAAAAATGAAACCAAGAAATATGCTTGGTTTATGCCCTCAAAGTAA
TTTCTCAGAAACTGAATGAGAGAATGAAGCCTATATATACATAGATCCTCAGCATTATATAATA
ATTTAAGACAATGAAACATCAAACTTAGTATTACATGTGAGGAAAATGAAATCTAAAAGAGATT
CCAGCCTACATAAATTAATGGCAGAATTGGAAATAGAAGTCAGGATTCCAGGTCTCATGGACGA
ACGTGATTATGTGATTTGGTGTTACCCAAACATTTAAACTACATTTGAGATATAACAGACTTTT
CTTCATGTGTTTATCGTGAACATGAAAAAATTAATTTGGATAATCTGGAAATCTCAAGCAATAG
TTTAAGCCTCAAGTTGACTGAAGTAGTTGAAGCTAGCCTTTCTTTCTTTTTCCTCTTTTTTTTT
TTTTTTTTTTAGACGGAGTCTTGCTCTGTCACCCAGGCTGGAGTGCAGTGGCGCAATCTTGGCT
CACTGCAAGCCCCGCCTCCCAGGTTCATGCCATTCTCCTGCCTCAGCCTCCCGAGTAGCTGGGA
CTACAGGCGCCTGCCACCATGCCCAGCTAATTTTTTTGTATTTTTAGTAGAGATGGGGTTCACC
GTGTTAGCCAGGATGGTCTCAATCTCCTGACCTCGTGATCCGCCCGCCTCGGCCTCCCAGAGTG
CCGGGATTACAGGCATGAGCCACCACGCCCGGCCTTGAAGCTAGCCTTTCTTAGAAATCCCAGG
CATTCTTAAGTATTAGAGGTCTCTTTTATCTGATTATTGCTTCTACTTAAATATTCATGGTATT
AAGGAATTTTTTTAAAAAATTATGAATTGATTTGATGTAATAGCTCAGAAAACTATAAGATTTT
AAGTGATAAGGTTTTCCTTTTGATTCCTGTAAGTCTAGTAATATCATATTTTGATATTAAGATG
TCATCCTGCTAGGTATTCTGCAAATGCTTTGATATCAGGTCAGATTTTTTTTTTAAAAAATGAA
CTCCCTAGGATTTCATCATCATGGCCAATTAAAAAGTTCAGAAATTAAAAATCATTTTATCCAG
CAATTGATGAAATCAAGAGTCTTAAAGAAGAGGAAGTTACGCAGTGAAGAGGTAGATATGATTA
TATCCAGGATATTTTTGTTTATTTTTCCCCAGTAATCTCTGTCTGTTGCTAGTCTCCATGTTAA
ATAAATACAACATACACTGTACTTTTAAATATACCTAAAAATTGGCCTGGCGCGGTGGCTCACA
CCTGTAATCCCAGCACTTTGGGAGGCCAAGGCGGGCAGATCACAAGGTCAGGAGACCGAAACCA
TCCTGGCTAACACAGTGAAACCCCATCTCTACTAAAAAATACAAAAAAAAATAGCCAGGCGCGG
TGGCAGGCGCCTGTAGTCCCAGCTACTCGGGAGGCTGAGGCAGGAGAATGGCATGAACCTGGGA
GGCGGGGCTTGCAGTGCGCCAAGATTGCGCCACTGCACTCCAGCCTGGGCGACAGAGCGAGACT
CCGTCTCAAAAAAAAAAAAAAAACAAACAAAAAAAACCCTAAAAATCCACGTGTGTATTGTAAA
TAAATTCAGGCATGCATGCACGTTCTCCTTTTCAGAATGTTTAGACATAGTCCTCCAAATTTCA
AATATCCTAATGCATTTAAAACAACTTCTGACTTAAAAAATACAGTTTTATGGTGAGGTTTGGG
TTTAAACAACAACATACAATTTTAAATAATATAGCTATTATGTAAAGAATACACCTTTACAGTT
TATTTGTTAAATCTTTTTTAAATTGACCGAACAATTGATGGAGGTTATTTTTAAGAAAAAGAAT
TATCAGTACAGCTGTTTATGCAATAATGGCCTTTATCAAAATGGTTATTGCTAAATTTTATGTG
TTAATAATTTAAAATAAAATAGTTTTAAAGGCTATTTATGTATTCTGTTTTTACTATTTTATTT
ATTTTAATTTTTGAGACAGGGTCTTAACTCTGTCACCCAGGCCTGGAGTACAGTGGCACGATCT
CTACTGACTGCAGCCTTGACCTCCCACCTCAGCCTCCCAAGTAGCTGGGATGACAGGCGCATGC
CACCACCCCCCACTATTTTTTATTTATTTATTTATTTATTTATTTATTTATTTATTAGTTTTTT
GTAGAAACAAGGTTTCATCATGTTGCCCAGGCTGATCTCAAACTGCTGGGCTCAGGCAATCTGC
CTGCATTGGCCTCCCAAAGTGGTGGGATTACAGGTGTGAGCCACGGCGCCCAGCCTTTATTCTT
AAGTGGTACAGTATTTTATGAAATCACTTATGTGTACCAGAATGGTAATCTAAAATAGGTAATA
ACAAAATTTTTGCCTACAGAATTCAGTTACCAACTACTTTTCAAATTATTCTTTTTACATATTT
ATATTTGAACACATTCTGACTCTGAAAAATGTATTTGTTGTAAAAAATGGAAAAATATGGAAAA
GAATAAGGAAAATAAAAATCACTCATAATCCTAAAAAAAAAATTTTTGGTCGTCATTGAAGAGA
GTGAGTAAAGCATAGTAGTTAAGAGCTTGTTTCCTGGATTAGTATCTCTGTTCCACAACTTACT
GTGACTTACTGTGACTTGAAGTTATTTCACCTTTCAGTATCTTGGGGTCCTTATCTGTAAAGTG
GAGTTAATGTTTCCTTCCTCATAAGGGTGTTGTGAAGATTAAATTAGTTGCTATGTGTGAAATG
CTACAAAAGTGCTATATATTATTGTTGCTGTATTCATGGAATATTTTTGTTCTATAATGCTATG
GTAGGTTTTCAGAAAATATTTTAGAACCAATAAAATAACAGCAACAACAGCTAGTACTTTTAAG
CACTAACTTTATGCAGGGAATTAATCTAAGCAGTTCATACATTAATTTATTACTATATTATTAT
GGTTATTTTACAGATGAAATAAGAGAGGTAAAGTTGTATGTTTGCATTTACAATAGAACATATT
GCATTGCATTACATTCTTTTGGCACCAAGGTGGGGAGCATAGAATAGAGATCAGTGGAAGTGGA
CAGACATGTAATATTTTATGGAGAATAAAGATAAATTTGCTTCAGTTATCATTTGACATGTCTT
AGGTAGAGCTAAGACTAGATAGAACACAGGGCCTCTCAATCCTGTAGAAATTCACCACCACATC
CTTTAAAAGGTTTTACATTGATAAGGAGTCAGCTCTCATCTATGAATGTTAGTGGGGAAAAGCT
GTAGAATGCACAGTCTAAATACCAGATAACCTTTTTTTTCCCCACTTTGGAATACATTATATTT
GATAAAAGCAAGTATACCTTTGGATGTAAAGGTAAATATAAACACTGACTTACATCTTTAACAC
CTAGCCAGTTGGGAACTGGCATAAGGAAACTCAGAATGGATTACATGGCAATATGGAATGTTGG
CCTGGTCCCACATTTGAGCTAAGATGTTAATTCTAATTTTAGTCTCCTCAGAATTCTGTATACT
CCGTAGCTTGAAACAGGTTTCCAACTGAAACTATTTTTTTCCCCAAGGTTATTTTCACATCTAC
ATTTGTTTGACTCTAGATCTTGGAACTAGTCTATATCAAGTTCCTTTGGGGACACAAAATTGCA
AATCTGTTGTTCAGAGAGCAAAACTATTTTAAAAGGTAACACTCGTTCTCATATATTCTTAATA
AGCCTTCACAACTTACAGTATGTTGAGGAAACTGAGGCTTAATTGAATCGCGTGCTAAAGTTTA
AAGGTTAAAAGTTATGGTAAGTGAGAGTTGGGACTAGTAAGGTGAATTCTGACTGACTCTTGGT
CTTTCCATAACAAAACATTGTCTAAAAGTAAACTGTGTGGCCTAAAAATGATTTTGTAAATATT
AGAGATCGGTGTTGAACAACAACAGACATTATTTAAAGCTAAAGACTAAATTCACAGAATAGGA
CCCTAAAGTTGAAGATACCCAGAAGATAATTTCAGCTAGTTTTGTCAAACTCTTTGACTGCAAT
CCCTAGTATTTTACATAGCATCCCAGTACATGCTTATCCATCTGTACATCTAATTGAAACAAAA
GTTTCACAAAACAGTAGGTTCCATTACTATGTGGAATGCATTCTGATTTTCCCATTGCTGATTG
CAAACCACTAAATGGATTAGACTACCCACTAATGAGTTGCAGGTGACAAGTTTGAAAAAACAAT
GCTCTCGGGCAACCTGACCTGATGCGTTAATTTATGAGATTTTATTTTTTGTAGACAGACTAGA
TTGTCCAAATGAGTATTATTACTTTAACTACTATAGTGCTGTGTTACAGTTGTTTCAGTGATTA
TTGCATTTTTCAAAACCATTTTTAAAATTCTGTTTGAAATAGTTGGAATAACCTGAAAACATTT
AATATCCTGTTTTGGTAAATTTTCATCTCCAAGATTAAATTTTTTTAACCAGCCAACAGATGTT
TGAAGGCAAGTTAGGAGATATGATGGATACTGGATACAGTATCCATCCATGTACTAGATACAGT
ATCCATCCATGTACTGGATACAGTCATACTGTTAGCAAATTTTAGATACATAAAGTAAAACAAA
CAAAATGCAGTATAAACAGTTAATATTGGAATCTAGTGTTGATTTTATGATTAATCTCAATGAG
GTTACTGTATATTCATAATTCTATAGGTACACAAAAATATTTGGTAAAAGATTTATAGTTGAGA
AAATGCTAGACACTAAAGTTTGTTAACTTACAATTTAAGAATAAGGGCTGGGTGCAGTTGCTCA
CGCCTGTAATCCCAGCACTTTGAGAGGCTGAGGTGGGCGGATCATGAGGTCAGGAGTTCAAGAC
CATCCTGGCAAACGTAGTGAAACCCCATCTCTACTAAAAATAAAAAATTTAGCCAGGCGTAGTG
GCGCCTGCCTGTAATCCCAGCTACTCAGGAGGCTGAGGCAGGAGAATTGCTTGAACCCAGGAGG
CGGAGGTTGCAGTGAGCTGAGATCGCGCCACTGCACTCCAGCCTAGGCGACAGAGTGAGATTTC
ATGTCAAAAAAAAAAAAAAAAAGAGTAAGGACATGAGTAAGGTTATGTCTCACCTGTTTTTAAG
GAAATGTGGATATAAGATGGGTTCTAGTCCATTAAAAGGTGGTAATTTATACTAAGTCTTACTG
TGAGAGACCATAAACTGCTTTAGTATTCAGTGTATTTTTCTTAATTGAAATATTTTACTTATGA
CTTAGTAGATACTAAGACTTAACCCTTGAGTTTCTATTCTAATAAAGGACTACTAATGAACAAT
TTTGAGGTTAGACCTCTACTCCATTGTTTTTGCTGAAATGATTTAGCTGCTTTTCCATGTCCTG
TGTAGTCCAGACTTAACACACAAGTAATAAAATCTTAATTAATTGTATGTTAATTTCATAACAA
ATCAGTAAAGTTAGCTTTTTACTATGCTAGTGTCTGTTTTGTGTCTGTCTTTTTGATTATCTTT
AAGACTGAATCTTTGTCTTCACTGGCTTTTTATCAGTTTGCTTTCTGTTTCCATTTACATACAA
AAAGTCAAAAATTTGTATTTGTTTCCTAATCCTACTCCTTGTTTTTATTTTGTTTTTTTCCTGA
TACTAGCAATCATCTTCTTTTCATGTTTATCTTTTCAATCACTAGCTAGAGATGATCGCTATGG
AAAATCCTGCAGACTTGAAGAAGCAGTTGTATGTGGAATTTGAAGGAGAACAAGGAGTTGATGA
GGGAGGTGTTTCCAAAGAATTTTTTCAGCTGGTTGTGGAGGAAATCTTCAATCCAGATATTGGT
AAATACATTAGTAATGTGATTATGGTGTCGTATCATCTTTTGAGTTAGTTATTTGTTTATCTTA
CTTTGTAAATATTTTCAGCTATGAAGAGCAGCAAAAGAAGGATTTGGTATGGATTACCCAGAAT
CACACATCATGACTGAATTTGTAGGTTTTAGGAACTGATTTGTATCACTAATTTATTCAAATTC
TTTTATTTCTTAGAAGGAATATTCTAATGAAGGAAATTATCTCTTTGGTAAACTGAATTGAAAG
CACTTTAGAATGGTATATTGGAACAGTTGGAGGGATTTCTTTGCTTTTTGTTGTCTAAAACCAT
CATCAAACTCACGGTTTTCCTGACCTGTGAACTTCAAAGAACAATGGTTTGAAGAGTATTGAGA
GACTGTCTCACAAGTATGTCATGCTCAAAGTTCAGAAACACTAGCTGATATCACATTAATTAGG
TTTATTTGCTATAAGATTTCTTGGGGCTTAATATAGGTAGTGTTTCCCCCAAACTTTTTGAACT
CCAGAACTCTTTTTCTGCCCTAACAGAAGAGTTGTTATTGAACACAGTTTGGGAAAGGCTGATG
GGATTTGGAAATTTGAAAGTGAAGGATCAGAATTTTAGTTTTTTCCCTTTTGTGATAAAGTAGA
ACAGGGAAAAGATGCAGTCTTTTGGGTAGTCTACTTAACTTCATAATTCTGAACTGGTTCAGTT
TCTACTGTAAATATAACCACTTAGTAACTGAGCTTGCTTACGTTTAAAATTGAGTACATGACAA
TTACAGGAAAAGGTTTCCACTGAAGGTACCATCAGAATTGTGAGGAGTGTGCATAGAATAATGT
ATGTCATTTCCCTTCAGCTTTGAGATTTGAGCTGTTATAGCCTGTTGATTCTAATTGAGTTGAC
CTTTCTGTTACTGTTCTTAGTCACACACACACACACACACACACACACACACACACACACACAC
ACGCATCCCTTATCTATAATCTAGCTAGTGTTTTATTAATAACTAAAAAGCTATGCCATTTGTA
TGTAGTTTGTTCTAAGTAAATCAGAGATACATAAGACGACGCCCTTTTTGAGATAGAAAATTAT
AAACTTCATAAAGTTCTTAAATTTGGTAAACCTTAGCTCTAGCTTTTGATGTATCTAGAAATGT
TAAACCTTAGCTATAAAGCATACTTGCATTATATGCAGAAATACTTGTAAGAAAAAACATAGAT
TAAGCAGTTCCAGTAAGATAACTGAAGTGATGGCAGTAGAAGTATCAAAAAGGAGTATTTTACC
AGGAGGTTATGGTGCTTTTCTCCCTGGAACATGAGAAAATGTGCCTAAAATGGAACTTCAGAGT
TATATTCTGATTAACTTATAGCTTGTTGCTCTTGGTTCCAAGGAAGGGCATTTGTGACATTTTA
TTAAATTCATTAATTTTTTAGACACACCATTGTCAGCTTGAACAAATTTATTAATTGTAATTAT
TTGTCAGCTGTTCTTGATCCTGTTAATACCATACTTATAACTAAAAGCATTTCCATGGATGTTG
TAACTTGGCCTGTAAAAAAAATGTTTAGATAGAAACCATGAAACTCAAATATGAATTGTTTAAT
TTTCAAACCATTTTGCATTCAGAAAATGTCCTAAGCTTAATTCATACTCCTAGTGATCAAGGAA
ACATGTTAAAGCTCCTTATTTTTAAACTTAAAGTGACAATGACATTTTCAAAGATTTTAAAATT
CTTATAAACAGGTTAAAATACTTATATACTGTATAATTTGATTTCTGATTTCTAAGCTCTACTT
TTCTATTGGAAATTACAGATTTTTTTCAGACTTAATTCTTAAGATGTTTTCATTGTTTCACAGT
AGCAACTAAACATGTAGTAAAATGATTTAAATTCAATTAAAATTTTTTTCCTTAGTCATTTAAA
AGGGAAGAAATCAATTTTTAGTAGTACTCATTCCAAAGATTCCAATTTTCCTTTTTTTTAATCT
TTTATTTTTTGGTGGAGGGAGGCAGGATCTGGCTCTGAGGCCCAGGCTGGAGTGTAGTGGTTCT
GTCTCGGCTCACTGCAACCTCCACCTCCCAGGCTGAAGACTCAAACCATCCCCATGCATCACCC
TCTCAAGTAGCTGAGACTATAGGCACATGCTACCACACCCAGCTTATTTTTTGTGTTTTTGTAG
AGATGGGGTTTCTCCATCTTGCCCAGGCTGGTCTTGAACTCCTGAGCTCAAGTGATCTGCCTGC
CTCAGCCTCCCCAAAGTGCTGGGATTACAGGTGTGAACTACCACACCGGGCCCCAATTTTCCAA
TGAGTGATATAAAAAAGGCCTCCACGCAGGCGCCTGTAGTCCCAACTACTCCGGAGGCTGAGGC
CAGAGAATGGCATGAACCCGGGAGGCGGAGCTTGCACTAAGCTGAGTGCTGCTGCACTCCAGCT
TGGGCGACAGAGCGAGACCGTCTCAAAAAAAAAAAAAAAAAAAAAAGCCTCCATGATTGGGGCT
TGCATAGTGAAGACCATGTGAAATTGAAAGACTACGAAACTACTTTTCTTTTACGTATTGGCCC
ATAATTAACATGTGTATTGAATAGCTTTGTTTATCTAAGTTCATCAGATTTATCCAGGTTTATG
TATTTCAGATCATCTGATTTTATTAGGAAAATGCTAGAAAAATTTCATGGCACCATTGTCTAAT
TTTGAAAAAACGAACCTTTCTTTACTGTGATTAAAAATTGTTTTTTAGGCCAGGTGTGGTGGCT
CACGCCTGTAATCCCAGCACTTTGGGAGGCTGAGGCTGGCAGATCACGAGGTCAAGAGGTTGAG
ACCATCCTGGCAAACATGGTGAAACCCCTTCTCTACTAAAAATACAAAAATTAGCTGGCGTGGT
GGTGCACACCTGTAGTCCTAGCTACTCAGGAGGCTGAGGCAGGAGAATTGTTTGAACCTAGGAG
GCAGAGGTTGCAGTGAGCTGAGATCGTGCCACTCCAGCCCACCCTGGGTAACAGAGCGAGACTC
CATCTCAAGGAAAAAAATGAAAAATTGTTTTCAAAAATAGTACGTGTGGTACAGATATAAGTAA
TTATATTTTTATAAATGAAACACTTTGGAAATGTAGCCATTTTTTGTTTTTTTATGTTTATTTT
TCAGCTATGGGTGGATAAAGCATGAATATAACTTTTCTTATGTGTTAGTAGAAAATTAGAAAGC
TTGAATTTAATTAACGTATTTTTCTACCCGATGCCACCAAATTACTTACTACTTTATTCCTTTG
GCTTCATAAAATTACATATCACCATTCACCCCAATTTATAGCAGATATATGTGGACATTGTTTT
CTCAAGTGCTAATATAATAGAAATCAATGTTGCATGCCTAATTACATATATTTTAAATGTTTTA
TATGCATAATTATTTTAAGTTTATATTTGTATTATTCATCAGTCCTTAATAAAATACAAAAGTA
ATGTATTTTTAAAAATCATTTCTTATAGGTATGTTCACATACGATGAATCTACAAAATTGTTTT
GGTTTAATCCATCTTCTTTTGAAACTGAGGGTCAGTTTACTCTGATTGGCATAGTACTGGGTCT
GGCTATTTACAATAACTGTATACTGGATGTACATTTTCCCATGGTTGTCTACAGGAAGCTAATG
GGGAAAAAAGGAACTTTTCGTGACTTGGGAGACTCTCACCCAGTAAGTTCTTTGTCATTTTTTT
AATTCAGTCTCTTAGATTTTATTTAAATGCAAAAATTTAATTTATGTCAAAATTTTAAAGTTTT
TGTTTAGAATCTTTGTTGATACTCTTATCAATAAGATAAAAATGTTTTAATCTGACCGAAGTAC
CAGAAACACTTAAAAACTCAAAGGGGGACATTTTTATATATTGCTGTCAGCACGAAGCTTTTGT
AAGATTGATTTCATAGAGAAGTGTTTCTAAACATTTTGTTTGTGTTTTAGTGAAATCTTAAGAG
ATAGGTAAAAATCAGAGTAGCCCTGGCTAAGGGTCTTGGTAGTTACAACGAGTGTGCCTGCTCC
TACCACCCCCACCCCCACCTTGAGACACCACAGAATTTCTCATAGAGCACAGTGTGAATTCTAT
TGCTAAATTGGTGGTATGGGGTTTCTCAGCAGAGAATGGGACATCACAGTGACTGACAATCTTT
CTTTTATAGGTTGGAAACTATTTGGGGGACTGGAGGGATACTGTCTACACTTTTTACAATTTTT
ATTGATAAGATTTTTGTTGTCTTCTAAGAAGAGTGATATAAATTATTTGTTGTATTTTGTAGTT
CTATGGTGGCCTCAATTTACCATTTCTGGTTGCTAGGTTCTATATCAGAGTTTAAAAGATTTAT
TGGAGTATGAAGGGAATGTGGAAGATGACATGATGATCACTTTCCAGATATCACAGACAGATCT
TTTTGGTAACCCAATGATGTATGATCTAAAGGAAAATGGTGATAAAATTCCAATTACAAATGAA
AACAGGAAGGTAATAAATGTTTTTATGTCACATTTTGTCTCTTCATTAACACTTTCAAAGCATG
TATGCTTATAATTTTTAAAGAAGTATCTAATATAGTCTGTACAAAAAAAAAACAAGTAACTAAG
TTTATGTAAATGCTAGAGTCCACTTTTCTAAATCTTGGATATAAGTTGGTATGAAAGCACACAG
TTGGGCACTAAAGCCCCTTTTAGAGAAAGAGGACATGAAGCAGGAGATAGTTAATAGCTAAGTG
TGGTTGTAGTATAAAGCAAGAAGCAGGGTGTTTCTTGTATTAAGCTGTAAGCAGGAACCTCATG
ATTAAGGTCTTTATCACAGAACAAATAAAAATTACATTTAATTTACACATGTATATCCTGTTTG
TGATAAAAATACATTTCTGAAAAGTATACTTTACGTCAGATTTGGGTTTCTATTGACTAAAATG
TGTTCATCGGGAATGGGAATAACCCAGAACATAACAAGCAAAAAATTATGACAAATATATAGTA
TACCTTTAAGAAACATGTTTATATTGATATAATTTTTTGATTAAATATTATACACACTAAGGGT
ACAAAGCACATTTTCCTTTTATGATTTGATACAGTAGTTTATGTGTCAGTCAGTACTTCCACAT
TTTTGCTGAACTGGATACAGTAGGCAGCTTACCAAATATTCTATGGTAGAAAACTTGGGACTTC
CTGGTTTGCTTAAATCAAATATATTGTACTCTCTTAAAACGGTTGGCATTTATAAATAGATGGA
TACATGGTTTAAATGTGTCTGTTTACATACCTAGTTGAGAGAACCTAAAGAATTTTCTGCGTCT
CCAGCATTTATATTCAGTTCTGTTTAATACATTATCGAAATTGACATTTATAAGTATGACAGTT
TTGTGTATATGGCCTTTTCATAGCTTAATATTGGCTGTAACAGAGAATTGTGAAATTGTAAGAA
GTAGTTTTCTTTGTAGGTGTAAAATTGAATTTTTAAGAATATTCTTGACAGTTTTATGTATATG
GCCTTTTCATAGCTTAATATTGGCTATAACAGAGAATTGTGAAATTGTTAAGAAGTAGGTGTAA
AATTGAATTTTTAAGAATATTCTTGAATGTTTTTTTCTTGGAAAAATTAAAAAGCTATGCAGCC
CAATAACTTGTGTTTTGTTTGCATAGCATATTATAAGAAGTTCTTGTGATTAATGTTTTCTACA
GGAATTTGTCAATCTTTATTCTGACTACATTCTCAATAAATCAGTAGAAAAACAGTTCAAGGCT
TTTCGGAGAGGTTTTCATATGGTGACCAATGAATCTCCCTTAAAGTACTTATTCAGACCAGAAG
AAATTGAATTGCTTATATGTGGAAGCCGGGTAAGAAAGCAGGTGTCTGCAAAAAGTCATGTATC
GATTTATTGTTTGTAATGATACAGTAGTATAGCAGATAACTAAGACATATTTTCTTGAATTTGC
AGAATCTAGATTTCCAAGCACTAGAAGAAACTACAGAATATGACGGTGGCTATACCAGGGACTC
TGTTCTGATTAGGTGAGGTACTTAGTTCTTCAGAGGAAGATTTGATTCACCAAAGGGGTGTGTG
ATTTTGCTTCAGACCTTTATCTCTAGGTACTAATTCCCAAATAAGCAAACTCACAAATTGTCAT
CTATATACTTAGATTTGTATTTGTAATATAATCACCATTTTTCAGAGCTAATCTTGTGATTTAT
TTCATGAATGAAGTGTTGTTATATATAAGTCTCATGTAATCTCCTGCATTTGGCGTATGGATTA
TCTAGTATTCCTCACTGGTTAGAGTATGCTTACTGCTGGTTAGAAGATAATTAAAATAAGGCTA
CCATGTCTGCAATTTTTCCTTTCTTTTGAACTCTGCATTTGTGAACTGTTACATGGCTTCCCAG
GATCAAGCACTTTTTGAGTGAAATGGTAGTCTTTTATTTAATTCTTAAGATAATATGTCCAGAT
ACATACTAGTATTTCCATTTTACACCCTAAAAAACTAAGCCCTGAATTCTCACAGAAAGATGTA
GAGGTTCCCAGTTCTATCTGCTTTTAAGCAAATGCCCTTACTACTCTACTGTCTACTTCTGTGT
ACTACATCATCCAATTCTGAAAGACATAGGCTTCCCCATCCCCTGCTAAGACTGGTTCAAGTGG
CAGCTACTGATGGATTGCAGTGAGAAGGCATGCAAACACGTACCTTCCTGGAAGTTGTCTCCAA
AGGCTATTGCTCTAAGACTCAAGTATATAAACACTAGAATGAATATCAACTCTATCTAGCAATA
AATGTTATTTTTATATTACAGTTGACCCTTGAACAACACAGCTGTGAACTTCATGGGCCCTCTG
ACATGCAGATTTTTTTTCTCAACTAAGAGCAGATTCAGTATTGGTGGGACTCAGAACCTGCATA
TACAGAGGGCTGACTTTCATACATGCCAGTTTCACAGGGCCAACTGCAGAACTTGAGCGTGCAT
GGATTTTGGTATACACACGTGGTCCTGGAACCAATCCCTGTCACATATACCAAGGGATGGCTGT
ATGTTACTTTATATTCATTTGTTCTGTTATTTTATAAGGTTGTTCGTCGTGGTATGTGGGAATT
CACCAGTATTTCTTCTTTCTGGTGCACCGTTGGTCATTTCTGGCAGCAGTGGTGAATGTATTTA
CTCTTAGCAACCTCTGTGCTGCTACCTGTTCTGAGTTTCAAAGGTGATTCATTAAAGGGTTGGG
ATAACATGGTGATAGGAAAAACCCCCCTCATCAGTCACAAGGAGTATAACAGCAATATCTCTGT
AATATGATTGATCATAGATATAATTTCTAGTAGGAAAAAAAGTCATATCTTGATGCATCTCTGA
GAATAGTTGAACATATCTTGTGCTATTCTTTATAGAGAAATTATCTTTGAAATTAAAGTCTTAA
TTTTACTTCTAGCTTTTTATAACAACATAATCCCTACTTGGTATGTATCTTAAGATCATTTTTA
AATGTATGATTTGAAGGGCAAACTAGTGTTATGTGAAAAATGACAGATAAAGTAGCTTCCAACT
CATCCTCAAGAGTTGATGATATTCTAAACCTTTTCTAACTAAATTCAGCTTCTTAATTTTCTCA
ATATAAATATGATGAAAATATTAATTCATTAAATAGTCTACAAGTATTCGGTAGTTGAAGACTT
AAAGTAGTGCTTGTAATAACAGAAGAGAAAAAAGACATTACAGGCGTATCTCACTTTATTGCAC
TTTGCAGATACTGAGTTTTTTTGGTGGCAACCCTGCATCAAGCAGGTCTACCAGCACCATTTTT
CCAACAAAATGTGCTCACTTCATTAGCATTTTTAGCAATGATTTTAAATTAAGATAATGTACTT
ATTTTTAGACAATGCTGTTACACACTTGACTACAGTATAATGTAAACGTAACTTTTATAAGCAC
TGGGAAACAAAAAAATTTGTGTGACTCACTTTACTGCCATAATCACTTTATTTGCCATGGTCTG
AAACTGAACCGGCAGTATCTCTGGGGTATGCCTGTATAGATATTTTGGTTGGTATTTATTTATT
GTATGCAGAATTCATAAAAATAAAAACTGCGAGGCTGTTTAATACATTTCAACTAAAAGTTGCC
AGCATCATTAATATGTAAACCACTAGAAATAAGATTTTGTTAATTTTTTGTTTGTTTGTTTAAA
CAGTCTTGCTCTGTCACTGAGGCTGGAGTGCAGTGGCGCAGTCTCAGTTCACTGCAACCTCCGC
TTCCTGGGTTCAAGTGATTCTCCTGCCTCAGCCTCCTGAGTAGCTGGGGTTACAGGTGCACACC
ACTACACCTGGCTAATGTTTGTATTTTTAGTAGAGATAAGGGTTTTGCCATGTTGGCCAGGCTG
GTCTCAAACTCCTGACCTCTGGTGATCCGCCCGCCTCGGCCTCCCAAAGTGCTGGGATTACAGG
CGTGAGCCACCATACCTAGCAAAATCAACTCTGAATCTGAATCAAACCTTTGAAACAAAAATTA
CCAATCAAAAAAACGTGCAATCCCTGCTCCTAACTTTGAAAAAGTGACAAGGAAGGACATTTGG
AAAGATGTCTCTAAGCAGTCAACAAGAAATGAAATCAGGGAGAGCTTTTTCAGCACCTGAAAAA
ATACATGCAAAAACGCCCGAGGCGGGCAGATCACCGGAAGCCAGGAGTTTGAGACCAGCCTGGC
AAACATGGGGGAAACCCCGTCTCTACTAAAAATACAAAAATTAGCTGGGTTTGGTGGCAGGCAG
CTGTAATCCCAGCTCCTCGGGAGGCTGAGGCAGGAGAATCGCTTGAACCTGGGAGGCGGAGGTT
GCAGTGAGCCTAGATCGTGCCACTGCACTCCAGCCTGGGCAACAGAGTGAGATTCCATCTCAAA
AAAACGAAAAACAGATTCAGAGTTTATTTCCTTCCCAAATACTTCATAGATGTTGGTTTGTAGG
AATAACTTAAAAGTCTCACTCGCTTTCTGTCTTTTCTAGTAATTTTTATCGTGGATTCTGATTG
CCTAGATCTGCTAATTCATTGAGGGTTACAAAATTGTGATACTCGAATTCTTTATTCCTCCTTC
CTTTATTACCTGTAATACTTCTTAAGAAAAACTCTTTCTCATCAACTCTATGACTACCCTAAAT
TACAGATCCACATATAGGATCTGTATACATGTATATAAATATGTACATATCATATACGATAATG
CTTAATTCTTTCCCATTACTTACTAATTTTCAAAATAATGTGATTCCCTAACTGCTCTCTGAAT
GTTATCAATGTGGTGGTAATAATGGTGGTGTCGATGTTGTGTGGGGTAAAATTTGAATACAGTG
AAATACCCAGGGCTTAATTGGGTTAGTTTTTAAAAATTACATTTATGTAACCATATTTCAGTCA
AGGTGTAGACTATGTCCATCAGCACAGAGTTCCCATGTTTTCCCTTCCAGTGAATCTCCATCCC
CCATTAGAACAGACAATAACTGTCCTAATTACTGTCACTGTAGCTTACTTTTGTCTGCTCTAGA
ATTTCTTATGAATGGAATCTTATGTACTTGTACTGCATGAAGACTCTTTAGATTCATCCATGTT
ATTGCATTGATTTATAGTGCCTTTTAAAAAATTATAGAGTAGTGTTGCATTATATGAATACGCA
AAGTGTTTTTTTTTTACCTGTTGGTGGACAGTTGGATTGTTTCCAGTTTGGGACTATTGTGAAT
AAAGCTGTGTAGTCTTATTGTGGAAATGTTTTATTTCTCCTGGGCAAACATGTAGGAGTGGAGT
TTCCAAAGCATATAGCAGGTGCATATTTAACTAAAAGTGTCTTTTCCTCATTTTTAATATTGGA
TTTTTGTATTTCTATTGACTATAGATATTTTCTTAAAATATAGCAGTATGTCTTATCCTTAGTT
TTGCTTTTTTTTGGTTTCAGTTATCTTTGGTCAGCAGTGGTCCAAAAATATTTATTAAGTAGAA
AATTCCATGAATAATTCAGGGTTTTTGATGTATGTGTGTGTTGGCGGGTGCAGGGCAGGGAGAG
TTGGTTGGTTGGTTCGTTGGTTTTTTTTTTTTTAAAGAGACAAAGTCTCTGTCATCCAGGCTGG
AGTGCAGTAGCATGACTACAGATTGTTATAAACTGAAACTCCTGGGCTCATGCAATCTTCTTAC
CTCAGTCCTCTGAGTATCTGGGCTCATGCAATCTTCTTACGTCAGCCCTCTGAGTAGCTGGTAC
TACAGGTGTGCAGCTAATTTTTAGTTGGTTATTTTTTTGTAGAAACAGGGTCTCACTATGTTGC
CCAGGCTGGTTTCAAACCCCTGGACTCTAGCAATTCTTCCACTTCAGCCTTCCAAAGTGCTGGG
ATTACAGGCGTGAGCCACTGCACCCAGCCAACAACTCTTAAGTTTTAAATTACATGCTATTCTG
AGTAACATGGTGAAATCTCCCACCTTTCCACCCAGGGCATGAATTATCCCTTTTTCCAGTATAT
CGTTGTTGTCTAGACTACCCACCACTTTTAGTCTACTGGTTATCAGATCGACTGTTGCAGTATC
ACATGCTTGTGTTCAAGTAACCCTTATTTTACTTAATAATGGCCCTAAAGCACAAGAGCAGTGA
TGTTGGAAATTCGAATATATATGCCAAAGAGAAGCTGGAAAGTGCTTCCTTTAAGGAAAGAATT
TAAAAATCATATGTTGAGGTTGCTAAAATCTATGGTAAAAATGAGTATTGTATCTATGAAATTG
TGAAGAAGGAAAAATAAATCATGCATAGTATATACAGGGTTCAGTACTATTTGCAATTTCAGAC
ATCACTGGAGGTCTTGGAACGTATTCCCCATGGGTAAGTGGGGAATACTGTATATTCTATATAA
AAGGCCCATTTCTGATAAGTAGTTTATGATTATTTTCTCCAAGTTTTCATTTTCTTAAGTGTCT
TCTGGTGAGCAGAAATTATTCATTTTTTGAGGTCTAATTTATTTTTTCTTTTATGGTTTTTTAT
GTCCTTTTATATCTTTGCCTAGCCCAAAGCCACAAATATCTTATGTTTTCTTCTAATTGTGTAA
TATAGGTTTAACTTTTATGTGTAAATCTGTGATCCACCACCAATAAAATTTTGTGATGCTGTTA
GACAGTGGATGTCATTCATTTTCTAAATAGTTTTATTTATTTGTTCCAGTACCATTTGTTAAAG
AGACTTTTCTTTCCCCATTGAATTGCCTTTGCACCTTTGTTGAAAATCAATCTGTGTGTGAGCC
GTGTTTCTGGATTCTCCATTCTGTTCTATTAATTTGTTTTGTCCTTTTGCCAAAATGACACTGT
TATGGTTACTGCAGCTTCAAATCAGATGCTGTGAGTCCCACAACTTTTTCATTAGGTTGCTTTG
ACTTTTATTTGCATATAAATATTAGAATCAGTTAATTTTTACAAACAAAAGGGCAAAGCCTGCA
GAGATTTTGATTTGGGTTGCCTTGACTGGGGCAGGTCAATTTGGAGAGAATTCACATTTTAACA
ATATATTCTTCCAATCCTTGAACACCGTACGTCTCTCCATTTATTTAGATGTTTGTTAGTTTAT
CTCAGCACTGTTTTATAGTTTTTAATGTTGAAATCATGTACTCTTTGGTTAGGAATAAACTTAA
AATATGTTTCTAATGCTATTAGTGGTATTTTTTAAATACTGCTTTTCACTTGTTTATTACTATC
TTGAAATAAAATCGATTTTTATATATTGATCTTATATCCTATAACCTTTCTAAATTGACTTATT
CTAGTAGTTATTTAGTAGATTTCATAGGGTTTTCTAGTAAACAGCTATGTCATCTACAAATCAA
GACAGGTTTCTTTCTTTCCAATGTTTATGTTATTTATTACTGTTCCTCTATTGCACTGGCTAGA
ACCTTTGGTAGAAGGTATTCTTACTTTATTCCTCATTTTATTGAGAGAGACTATAATATTTACC
ATTAACTGTGATGTTACTTGTAAACAAAAAGTTTGTATTTCTCTGGGATTAAAAACCAAGAGTC
CATTTGGTTTTTTACTGTTGAGTTTTAAGGGTTCCTTATGTATTTTCTAGGTATGTTCTTCCTT
CATTGAATTGTTTTTTTCACATACGTTAAAAATCACTTGAGAATATTTATATAGGTCTGCTTCT
GAGATTTATCTGTTTATCAGGTTTTGTTTCATTTATTTTGAGGCTCTATCATTTGGTTCATACC
CATTTAGAATTACTATGTCTTGGTGATTGGTCCTTTATCATTATATAAGTTTTTTCTGTTTTGC
GATTTTCTTTGCTCTGAATCTGATATGAGTGTAACCATTTGTTTTTTTTTAAATTAATGTTTGC
ATGATCTGTCTTTTACTATCATTTTACTTTCATCCTGTGTTGCTGAATTTGAAACGAATCTCTT
GTAAACAGCATATAGTTGTCATTTTAATAAAAACTGTTAGTCTCTGCCTTTTAATTTTTTTATT
TAGACCATTTATATTTAAGGTAATTATTGATATGTTAGGGAGAGGTCTGCCATTTTATTATTTG
TTTTCTGTTTCTGTCTTCTGTTTATGGTTATTTATTTATTGCCTTCCATGATTACTTGAACATT
TTTTAAGATTTTTAAGACCCTTGATTTATTTAGTGTTTGGGTTTTGTTTTGTTTGTTTGTTTGT
TTTTGTTTCGAGACACAGTCTCGCTCTGTTGCCCAGGCTGGAGTGCAGTGGCACAATCTCAGCT
CACTGCAACATCGGCTTCCCAGGTTCAAGCAATCCTCCCGCCTCAGCCTCCTGGGTAGCTGGGA
CTACAGGCACATGCCACCACACCTGGCTAATTTTTGTATTTTTATTAGAGATGAGGTTTCACCA
TATTGGCCAGGCTGGTCTCGAACTCTTGACCTTGTGATCTGTCCACCTCAGCCTCCCAAAGTGC
TGGAATTACAGGCATGAGCCACCATGCCTGGCCTTCTATGTGTTTTTTATAGTTGTTTTCACGG
TCATTGTGGGTATCACATTATAGATAATGTGACTTACCACCATTCACTGCCATCAATACCATTA
CATGGAATGAGATATGGAAACCTTAATTTCAGTTGGGTCTCTTTCCCCACTTTTAAATAGTATT
GTTTTGAGGATTAATGCTATTACAGTTTTTGTATCAGTTATCCAATATGACTTATAGAACTTAT
CAAGGGAGTGATGCTCTGTTGTATGTACCCATGTTTCTGCTATTTCCATTATTTTTTCCAAGAT
TCCATCTTTTATTACTTCCATTAGCCAGTCTTCAAAGGCAGTTCTGCTAGTGAGAAATTCTTTT
TGTTTTTCTCTTTCTGAAGATGACTTTATTTTCCCTTTATTCCCAAAGAATAGTTTTATTTGTG
CTATTTGGGGTTCGCTCAGCTTCTTGAAGCTGTAAGTTTGTGTCTTTTGCCAAACATGGAAAAT
TTTCAACCATTATTTCTTCAGGTGTTCTTTCAGCGTCACTCTTTTCTCCATTCTAGGCCTCTAA
TGATACAAATGTTGAGTCATTTGTTACTGTTCCACATGGACCTGAGGCATGGTTCATTTTTCAG
TCTATTTTCTCTCTTTTGTTCAGATTTCTCTCCTTTGTCATCTTCACTCTACTGTTGAGCCCAT
CTAATGATTATTTTTCTCCTATTGTATTTTTTAGGTCTATAATTTCCATTTGCTTTTTTTTCCT
TGTATCTTGTTTCCTTGCTGAGATTTTCTATTGTTTCGTTTTTGAAACAGTAGAACTTGTAATT
GCTTGTCAAATCATTTCTCTGATGGCAGCATTAAGATCCTTGCCAGATAATTTCAGCATCTTAC
TCATCCCAGTGTTGGCGTTTTCTTTCCTTCATGTAATTTTCCTGGTTCTTGGTATGACAGATAA
TTTTTGGATTGTGTCTCGGACATTTTAGTTATTATATCAGGAGACTCTGTACTTTTTAAAAATT
TTAACTTCAGTAGTAACCTGTTAAGGTATAGCATGTAGGTACTAGCATATATTTGTGAGCTGTG
GTTCCAGTGACCATTTTTCAGAGGCTTTATGGTGCTGTTTTGGTTTGTTGCTTTTATCTCAGTC
CACTGGGGCTCGCACTGGTTCCTAGTAGTGCTGCTTGAGGGGCCTTCTCCAGGCTGAGCTGCGC
AGAGTGTGTTTGGGTGAGGGAAGAGGACCCCCACTAGATTTTCGTGGGCTGGAGAGTGCTTCCT
GAGCCTTGTGCCTGTTGTGGCAGGCTCCCCCTTGCTGGTGTTCCTGGCAGTGCTCCTGACCACA
TTGTGTCCCTGGGCTGTGGAACACTTGCAGGAAAGGTGAACACTGCCAGGACCAATTATAGCAG
AATTCCTCCTGCCAGTACACAGCAGCACTGTGGCTCTGGACCAGCAAGGAGAGTCTCAAGCATG
GGTTTGTTTCTTTTTGATTTTAAGATTTTTCTTCTGCCTTCAGGTTCTGGTATTATTGTTCTGT
TTTATTCACCTTTATGTTTCTTCTAGTTTAGTTTTAGTTTCTTTGTATTTGATGGGATTTTTTT
CCTTGCATTTCTAATTACTCCTTAGTTGTGTCATGTCTTCCTTTTTTTCAGTTACCCTCATTCA
GAAATTTCCCATTTCTAATGATTTGTTATTTTTTAAATTCTTTTTAGCTCAGTTTGAAATTTGT
TTACAGTTTTCATGTTTTATTTGCATATTGTTTCATGTACTTTGATTTTCTGTAGGGACACCAT
TCTCATGTATATGCACATACTTTTTCTCTAATAATTACACTGTTTCACTCTGTTCTGTTATTTA
TTTATTTGTGTGTGTGTGTGTGTGTGTGTGAGTGAAATGAGTTTCTCATACTTGGACGAGGGAG
AGATGGGCCAGGATAACTTTTTTCGCTTTTTAGGGACGAGGAGAAGTCTGTGTTTTTGTAAAAT
GGTCAAAAATATGCCCTTGTGCTTTCTGAAACTGCCTTCTTTACTCCTCTATCCCACACATATA
TTTGAACTTTTTTTTGGTTTTTATTACTGACCTGGTCCATTCGGAAGTTATTCCCAGAAGTTTT
TCCTCGGTACAAGGTTTGTTTCTGGTGGGGGTTTGGTATTCGGGTGTTTTAAGGGCCCAGAGCT
TACTACATCATGATCCCTTTGTAATCATCTGCAAATTAGATCCTGTAGAACTTTTTAAAACTTT
AAGCCCTGTTTTCAGATTGGCCTGTGCTTTCCAGTGGGTTGGCAGTGTTCTCAGGGCTGTCAGA
AACCCAGTCAGTTACCTTGCATCTGCTTCCTTACTAACCAGTCTTGTAGTTGTCAGAGCTTTGT
TTCTGTCCAATTATATGCAATGGTTCATGGTTTTGTTGTAGATAATGTCTCTGGGTCTTTGGTT
TTCTCATTCAGTTTGCTGTTTTTGTAGGGGAGGTTGGGAGGGAATAAAACAAAAAATAATAATA
ATAATAACTATGTCGTTGCCATCATCATCTTTCCAGAACCTGCTACCTGCCCTTCACCTGATTT
TCGTATTTATCAGTATCATTACCTTAATCTGTTATCTCATTGGGGATTGCAAAATGGCTGTTTG
GGGAAAAAAAATTTCCATCTGCTTGTCCTACATATATACCTGGCATTCTTTTTTCTCATCATTG
GGTGCCATTTGATTACCTTGAAAAACAGTTCTACAGAAAAGGCATGATAAAATAGATTTAAGGT
TTTTAGAGTAAGGAGTTAATGTAATAATTTTAATAGTGACAATTTAATTTTGTTTTTTGGTTGG
TTTTTGCTGCCTTTTTGAATATTACGAACTAATGAATGTTTTTGTATTTAATACGTTTCACAGT
CATTACTCTAAATGATATTCAAATTGCTTAGCTATCGTGTGCTTTTAAAATGACCCTATTAGAT
TTTGAGATTTCTAACTAATCTCGAACATTCATTCTCAGGCCTGGAATAAGTTATTTTTCCAATA
AATTGTTGGAGAATGGCATTTAGAAGTCAGATTTTAGATGCAAAGTATGGCCTTTGCTTTTGAA
CGTTACTGCTTGTAGGCCTTATCAGAGGACAGAACTAGGAAAATAATCTTCCTAGTGTATCCTG
AGAATCATCCCACATTATTGCAGAGGTGTTTTCCATTTTGTTTTGTATGACTGAATACTGCTCC
TTTGTGTGGATGCAGCATAGCTTATCAATATTTATGGGCATCGAGGTTATTTTTAACCTTTTGC
TGTTAGTACTAATGTCACAGTGAATCCTCTTAATGTTCATGCATTATTCCGTACATATGTAAAG
TATCATTAAGATAAATTGTTAGAAGTGGGATTGTTGCATCAAATGGTAGATGCATGTGTAATTT
TGCTAGATACTGCCAAATCCTCCTTCACAGAAAAATAACATTTTGCATTCCCACCAGCAGTTTA
CAACAGTATCTCTTTTCTTAAAACTTTCCAAACAGAATGTGTTGTTAAATGTGGATGTTTACTT
ATTTGCCAATTGAGAAATGGTATTTTACTATAGTTACAATTTTTGAGCTTACCTCAATTATGAA
TGATAATGGACATTTTTTCTTGTAGAGCTGTTTATAGTTTTATATAGTCTTTTGCTCATTTTGC
TGCTCAGCTGGTCGTTTTCTTCACTTTTAGAAACTTTATATGTTAAGGAGATTAGTCCTTTAAC
GCTGGTAAATTCGCAAATATTTTTTCCCAGTTTGTCATTTGTCTTTTAACCTTGCTTATAGGAT
TTTTTTGCCAAATGAAAGGTTTTATGTTTATGTAGTTAGCTATTAATCTTTTTCGTTATTATGC
TTTGATATGCATTTCTTTAGTTATGTTTATTTTATTCATTTTCTTTGTTTTTTAATTTTCTAGA
GTCTGTCCTCAGTAGAAATTTTAGTCATACTGCTTCTTCTTTGGTCACCTTATAATAAAATTAG
CCTTTATTTCCAAAGTTACCTTGCATATTTCTTCAGTATTTTTTAGTATTCTTATTTTTATTTA
AGATGTGCTCATTTCTTTCTGAATTTGTATTTCTCATCACTGGTGGTCTGTCTTGTCTGCTGCT
GCATTCTCCTTTTGTGTTGGAATGTGGTGTTACTAGTTTCCCATTGTTTATGACTGAATTTTTC
TGGTCTACTTTTGTCTACCGGAAAGTTTTGCTTCTCATTCGCCAGACTCTTCCCATAATAACTT
TATTTAGACTTCCGAATTCTGTTTTTCTTGTTTATGTTTCTATAACAGATGTTACTTTTTTCTG
GACCTGTTATTACTAGACCTCATGGTGGAGCAGGGAGGAATTGTGTGGCTTTAGGGGTCTTGGT
TGAAGGGATCCCTCTTCTGTTGCTTTAGTAAGGGCAGAATCACCACTTTCTGATTTTTCAGCTC
CATGTAGCTTAATGAAGACTCTTTTTCATTAGCTGCTTCCACCCCTTTACAGCCGCTTAAGGGA
CACTTCTGCTTTTGAAGGTACCCCCCTTCCTTTCATGCATGGTGCCTTCCTGAGCCTGCCATTG
CTGGACCCAAGTACTTTTCTGTAGTCCTTCCTTTAACCACCCTGTTGCCAGTGCTCTAACCTAC
ACTCAGTAGTGCTCTGTTGAGGGGCAGGTGGTGGAGGGGGTGGGGTCTGGCCTTGGGTGATCCC
TAATCAGTATGATTCCGCACAAGCCACTCTTCGATGTAGCTCCTGCTGGACCCTGCTCATTTCC
CCACTTCTTTGGTATTTGGAGTTTATTGTTCTGTCTCCTAGTTTTACTCTAGACCTAAGTCATA
GGTTTTTTTTTTATTTTCCCCTTTTTGATCCGGGTAGATTTTTAGAGATGTGAGTAGGATGTGA
TCGATACAGCCACCACTTTTATGTGGAAACCTGGAAGCTTCTTCGTGTTTTCTTCAGCTTTCTA
AATATATGCATTCTTGTTTAATTCAAAATGTTAATGTAACACACATAACACTTATAGGAATAGA
GATGCATTTTGTTCCCTGTTTTCATTGTATAAAATTTTTAAAGGTTTCGTTTAAAAGTCCAAAG
AAACAGCTCCGTAATAATATAGCCAGACTGGTCATTGTGACCACTGCTGATCTGCCACTTCCTG
CAGGGTCTGTCTCTGTTCTTCGGTGGTGCTCTTTGGTCTACCTCCTTAGGTGTAGCTGGTTCAG
TTAAGCTTCAGAGCCCTCCTGCAGTGTGAGGAGGCTTTATTACCACCCACTGCAGGGCAGCTTT
TCTGCTATGTTGGAGGGTTCTGTGCTACAGCAGACTTGTTTTCTCTTGTCAGCCTCCCCAAAAG
GAGTATCATGAACTGGGGTCCGATTCCTCTCCTCCTTACCTGAGAGATGCTCCTGCAAGTTTCA
TGTCCAGTTTTCTGTTGTTGTGTGTTTCTTTTTTGTTTTCTCTTGCTGACTGGTGGATTTGTAA
AGGGGCAGGGAAGGGAAGTGGCAAGAGATTGGAGAAAGCCAAGAATTCTAATAAAACTTTTAAG
TTATTTCAGATAAATGGTGTTGCAGCACCAAAAATGCCAGCATTTTGGTAATAAAAATAGTATG
TTGGGATATTAGAATATCAAAAGGTAGAAAATTGTAATTACTGGTTTGGGATGTGTGATGTTCT
CTGCTAACATTCTGGAATTTTTTTTTTTTTTTTTTTTGAGATGAAATTTTTTTTTTTTTTTGAG
ATGGCTCAAAATGTTTCCCAGGCTAGAGTGCAATGGTGTGATCTCAGCTCATTGCAGCCTCCGC
CTCCCATGTTCAAGCAATTCTTCTGCCTTGGCCTCCCAAGTAGCTGAGATTACAGGCATCTGCC
ACCACGCCCGGCTAATTTTTGTATTTTAGTAGAGACAGGGTTTCACCACGTTGGTCAGGCTGAT
CTTGAACCCCTGACCTCAGGTGATCCACTGGCCTCAGCCTCCCAAAGTGCTGGGATTACAGGCA
TGAGCCACCACACCTGGCTCTGGAAAGAACTTTCTAAATAACTATTAAAATGCATTTTATATTA
TATAAGCAACTAAAAATACTTTTAATTTTTTTTTTAATTTTTTAATCTTAACATTATTTAGCTA
TAGGCCAGGCGCGGTGGCTCATGCCTGCAATCCCAGCACTTTGGGAGGCCAAGGTGGGCGGATC
ACGAGGTCAGGAGATCGAGACCCATCCTGGCTAACATGGTGAAATCCTGCCTCTACTTAAACGT
ACAGAAAATTAGCCAGGCGTGGTGGCAGGCACCTATAGTCCCAGCCACTTGGGAGGCTGAGGCA
GGACAATGGCGCGAACCCAGGAGGCGGAGCTTGCAATGAGCCGAGATCGCGCCACTGCACTCCA
GCCTGAGCGACAGAGCAAGACTCTGTCTCAAAAAAAAAAAAATTTATTTAGCTATGAACTAGTG
TATATATGTTTTATAGATGCCTGATTATTCAAATAAACTTCAAGATTGCACTTTATGAGCTTGT
ATTTCGTTTTTTAGAACTCAAACTAACATAAGTGTCATTAAAACATAGCTTTTATTACATAGAT
TTTAGGAATGCCACAGGATAAATCATTCTCTTAAAATAACTGCAGGTGGCAGAAATGTCAGAAT
AATTCTCCTCCTCCATACACAGCACATATTACTTGTTTAAAGTATTCTAGTTCATATAAAAATT
GAACTTTTGTATTACTGCTATTAGGTATGTAGTTGTTTGCATTTGGGGCCAGTTGGTTGGGGCA
GGGGGTCTTTTTTTCTTTTGTCCTTAATCTGTATCACTTTTTCCTCCCAAAGTTGAGTTAAAGG
ATGAGTAGACCAGGAGAATAAAGGAGAAAGGATAAATAAAATATATACCCAAAGGCACCTGGAG
TTAATTTTTCCAAATATTCATTTCAGTCTTTTTCAATTCATAGGATTTTGTCTTTTGCTCATTA
CTGACTGCATAATGTGATTATACCATAGTTTAAATAGTCACTTCCTGTTACTACACACTTGGGT
TTTCTCAATTTTTTACTATTGTAGTACTAATATTTTACTATATTGTAATCTAATCTAAATTTTT
ACGTATTCAGAGCTGTTCAGGATAAATTTGCTTGGAAATTTTTAAATCACCAGAAGTGATACTA
TCCTGATAATTAACTTCCAAGTTGTCTCTTAATATAGTTTTAATGCAAATCATAAGCTTATGTT
AGTACCAGTCATAATGAATGCCAAACTGAAACCAGTATTGTATTTTTTCTCATTAGGGAGTTCT
GGGAAATCGTTCATTCATTTACAGATGAACAGAAAAGACTCTTCTTGCAGTTTACAACGGGCAC
AGACAGAGCACCTGTGGGAGGACTAGGAAAATTAAAGATGATTATAGCCAAAAATGGCCCAGAC
ACAGAAAGGTAGGTAATTATTAACTTGTGACTGTATACCTACCGAAAACCTTGCATTCCTCGTC
ACATACATATGAACTGTCTTTATAGTTTCTGAGCACATTCGTGATTTTATATACAAATCCCCAA
ATCATATTAGACAATTGAGAAAATACTTTGCTGTCATTGTGTGAGGAAACTTTTAAGAAATTGC
CCTAGTTAAAAATTATTATGGGGCTCACATTGGTTTGGAATCAAATTAGTGTGATTCATTTACT
TTTTTGATTCCCAGCTTGTTAATTGAAAGCCATATAACATGATCATCTATTTAGAATGGTTACA
TTGAGGCTCGGAAGATTATCATTTGATTGTGCTAGAATCCTGTTATCAAATCATTTTCTTAGTC
ATATTGCCAGCAGTGTTTCTAATAAGCATTTAAGAGCACACACTTTGCAGTCTTGTAAAACAGG
TTTGAGTATTTTCTCCACCTTAGAGGAAGTTACTTGACTTCTCAGTGACCTAACCTCTAAAGTG
CATTTACTGATGTCCTCTCTGTGGTTTTGTTGTGGAAAGATTTAGTTAAATGAACTGTAAGAAT
TCAGTACCTAAAATGGTATCTGTTATGTAGTAAAAACTCAATGGATACAGTATCTTATCATCGT
CACTAGCTTTGAGTAATTTATAGGATAAAGGCAACTTGGTAGTTACACAACAAAAAGTTTATGA
TTTGCATTAATGTATAGTTTGCATTGCAGACCGTCTCAACTATATACAATCTAAAAATAGGAGC
ATTTAATTCTAAGTGTATTTCCCATGACTTACAGTTTTCCTGTTTTTTTCCCCTTTTCTCTATT
TAGGTTACCTACATCTCATACTTGCTTTAATGTGCTTTTACTTCCGGAATACTCAAGCAAAGAA
AAACTTAAAGAGAGATTGTTGAAGGCCATCACGTATGCCAAAGGATTTGGCATGCTGTAAAACA
AAACAAAACAAAATAAAACAAAAAAAAGGAAGGAAAAAAAAAGAAAAAATTTAAAAAATTTTAA
AAATATAACGAGGGATAAATTTTTGGTGGTGATAGTGTCCCAGTACAAAAAGGCTGTAAGATAG
TCAACCACAGTAGTCACCTATGTCTGTGCCTCCCTTCTTTATTGGGGACATGTGGGCTGGAACA
GCAGATTTCAGCTACATATATGAACAAATCCTTTATTATTATTATAATTATTTTTTTGCGTGAA
AGTGTTACATATTCTTTCACTTGTATGTACAGAGAGGTTTTTCTGAATATTTATTTTAAGGGTT
AAATCACTTTTGCTTGTGTTTATTACTGCTTGAGGTTGAGCCTTTTGAGTATTTAAAAAATATA
TACCAACAGAACTACTCTCCCAAGGAAAATATTGCCACCATTTGTAGACCACGTAACCTTCAAG
TATGTGCTACTTTTTTGTCCCTGTATCTAACTCAAATCAGGAACTGTATTTTTTTTAATGATTT
GCTTTTGAAACTTGAAGTCTTGAAAACAGTGTGATGCAATTACTGCTGTTCTAGCCCCCAAAGA
GTTTTCTGTGCAAAATCTTGAGAATCAATCAATAAAGAAAGATGGAAGGAAGGGAGAAATTGGA
ATGTTTTAACTGCAGCCCTCAGAACTTTAGTAACAGCACAACAAATTAAAAACAAAAACAACTC
ATGCCACAGTATGTCGTCTTCATGTGTCTTGCAATGAACTGTTTCAGTAGCCAATCCTCTTTCT
TAGTATATGAAAGGACAGGGATTTTTGTTCTTGTTGTTCTCGTTGTTGTTTTAAGTTTACTGGG
GAAAGTGCATTTGGCCAAATGAAATGGTAGTCAAGCCTATTGCAACAAAGTTAGGAAGTTTGTT
GTTTGTTTATTATAAACAAAAAGCATGTGAAAGTGCACTTAAGATAGAGTTTTTATTAATTACT
TACTTATTACCTAGATTTTAAATAGACAATCCAAAGTCTCCCCTTCGTGTTGCCATCATCTTGT
TGAATCAGCCATTTTATCGAGGCACGTGATCAGTGTTGCAACATAATGAAAAAGATGGCTACTG
TGCCTTGTGTTACTTAATCATACAGTAAGCTGACCTGGAAATGAATGAAACTATTACTCCTAAG
AATTACATTGTATAGCCCCACAGATTAAATTTAATTAATTAATTCAAAACATGTTAAACGTTAC
TTTCATGTACTATGGAAAAGTACAAGTAGGTTTACATTACTGATTTCCAGAAGTAAGTAGTTTC
CCCTTTCCTAGTCTTCTGTGTATGTGATGTTGTTAATTTCTTTTATTGCATTATAAAATAAAAG
GATTATGTATTTTTAACTAAGGTGAGACATTGATATATCCTTTTGCTACAAGCTATAGCTAATG
TGCTGAGCTTGTGCCTTGGTGATTGATTGATTGATTGACTGATTGTTTTAACTGATTACTGTAG
ATCAACCTGATGATTTGTTTGTTTGAAATTGGCAGGAAAAATGCAGCTTTCAAATCATTGGGGG
GAGAAAAAGGATGTCTTTCAGGATTATTTTAATTAATTTTTTTCATAATTGAGACAGAACTGTT
TGTTATGTACCATAATGCTAAATAAAACTGTGGCACTTTTCACCATAATTTAATTTAGTGGAAA
AAGAAGACAATGCTTTCCATATTGTGATAAGGTAACATGGGGTTTTTCTGGGCCAGCCTTTAGA
ACACTGTTAGGGTACATACGCTACCTTGATGAAAGGGACCTTCGTGCAACTGTAGTCATCTTAA
AGGCTTCTCATCCACTGTGCTTCTTAATGTGTAATTAAAGTGAGGAGAAATTAAATACTCTGAG
GGCGTTTTATATAATAAATTCGTGAAGAAATGTGTGCTCTTCAGTTCTCAAGTTTTATTATTAT
GGTATTTATTAAAGTTCTACAATTGTAATAACGTATCCATATGACAAGTTTTAAAGTGGTAATT
GAAATAGGTTATCAGATATAGAGTTGTTCACATCAAGTAGACTTTTAACAGAGTCAGAATGAAC
CTACCCTTAAAATTTTAGAGAAAAAAAATCGTCGGTTGCACAGAATAGCTGCTCTAGCTTGCTT
AATTATGCCGGGCATGTTGTCACTCCTCTTACTTTTGCTGCCTTTTCATTACTATTTAATGGAA
TGTCCCTGAACAATAAGGAAGAGCAAAACATAGACATTTTGACTACAGTGGATACTTCCTCTAC
CCCAAATGTTATGTTATAAAAGTACTTTTTTTGCCCAGGTACTCTATTATATATTTTGGTTTTC
TTTGAATTAGACCTCAATCTCCAGGAAGCTCTGGAGGGAAAAAAAGGAACCATAAACTAAAGTA
ACTGGTTTTCCAAATAAATGTAAACTTTTTTAACCTTTTATTATTATAGAACATTTCAAACATA
CATAAAACATGAAAACAGCCTGCAGCCAAACTTTTCTGGACCTTGGCAGCTCCAGCAAATGAGC
TGGTCATCCTAACCCTGGTTTCCTGAGACAACTACTTTTGGGGATGCTTGCTTGTATAGGGAGA
TGATAGATAGATAGATACATATCAAAAAGAAACTTGAAAAAGGCTTCGAACAGAACTTGCCGAT
GTTACTCATCCCTCCTCGGTACAGACATTCTGTTAGAACAGATGGTTGTAATGGGGCAGCATCA
CTCCCTATTCCCATTAAAAACATGACCATTCTGAGATTTCAGGCCCCTTCGAGGAATTTTTCTC
AAGAATTCCATACAAACTGTCAGAGGAAGAAATGTCGTCCCAAGACTAGTCCCTGTGTAGTCTT
GATCCCTTTACCTGCTTACTGTAATTTTTTTTCTCTGACTTTCCGCCAGTGTCTAAGGGCCATC
TGTACCCCTCCCACCCTTCCATGAGCCACTGTATAATCCAAAAACACACCTTCCATTTGCTCCC
TCACTTGTCTCCTTTTACCTCAACAGTGTTAGTGTGTAAAGAATTGCCTGGGGAGGGTAGAGGG
TTTTACAAGTACAGAGTCCCAAGTCTTTTCTCCCCTCCCCGTTAGTGGGTTGGCATTTATTAAT
AGGCACTGCAAGATTTTGATGCAGGTGTGGGGTAGATATCACTGTGAAAGGCTACTAATGCATG
GTAGTTTCACCGAAAGTACAGTCTTCAAGGATCCTTATAATCTCCTCATTGCCAAAATCAGTAA
CATCTATAACCTTGACAACACCCTAGCTTGGGCTGTTGCTTCTCTGGCTACTTGTTTTTCCATC
CCAGTTTACCCAGATTTTAGATGGATACTCCTCCAATTCTGCTTAAAGTCCTTACCTGTTCTTG
CCCAGTTCCCTCTAGTCAACCTCAGTCACTCCCAAAGATTCTTCCCCAATCCCATATCTTTTGC
TCAGACCTCTGTCTCTGGGATTTCTTTGTTACCTCATAGTCAGTAGTCCTAAAAACAAAATTCT
TCATCTTGCATCTCTATCGACCGACCATTCTTTGTTTCTTTTTAAGAAATGTAACCTTCACCCC
AATCATCAAAATCTCTCATTCCTCAATTGTCACAGGTTGTCACTTCTTTCCCCCTAAACCTCTC
CCATCATTCCTTCCTTCTTTATACTGCCCTTACAGGTCCTGATCCTCTGACTCACCCGGACATT
CTTAGCAGCTGCCTAATTTCACTCCCTGACTCTCATCTCCCTCTTCCAATCCTTTTCATTGTGC
TAGAACACTATCTTGCTAATTTTAATATCCTCTGCATTCAGTGCCTCCACAGATTGCCCCCTCA
CTGACCTTTCCAATTTGGCCTTGTGTTACACACTTTCCTCGCTGCATTCCTACCCAGCATCTTC
ACTTGTCATATTTCTCAAATACTCTTCATGACCAGATATTTAAAGTACCTCATCCATGAAGACT
TCATCAACCCCCAAAGTGCCTCCAATGCTTCCGTGGTAGCACAGCATAATGGGGTACATTTCTT
AGTTGAACAGTGAGGAGTCACTGGGATTCTTACAGAGTGTCTTAGGGTCTCCTCTTGATACTCT
TTCCACATTTCAGTTAAGGAATTTTCTTACATCTTTTTTTCTTCTAATAACCTTTGCCTGGAGT
GTAAGTAAATTCATGATAGAGACTGTCATTTTTAAAAAAAAAAACAAACCATTTTGCCTTTCAC
ATGTTATTACCTCCATCTTTCTGTTATCTTGTCAGTGTTGCCACCTAAATGATTGAAAATGACC
GAGCATGCAGTAGAATGTAGTGAAGTTTCCTATTTCATCCATTTTTTGCATAAGTGCATTGGTA
TCTGTATATCCATCTGCCCAAGCACCAGTTGTTGAAAAGACTATTATTTCCCCATGAATGGTCC
TGGTACCCTTGTCAAAGAACAGTTGACCATAAATATAAGACTTTCTGGATTCTCAGTCTTACTC
CATTGCCCTATATGTCTGTCGTTATACCAGCACCGCACTGTCTTGATGGCTGTAGCTTGGTAGT
AAGTTTAGATTTGGGATGTGCAAGTCCTCCATTTTTTTTTTTTTTTTCCCCAAAATCATTTTGT
CTGTTTTGGGTCCCTTGCATTGCATATGAATTTTTATATGATCATTGTGTCAATTCCTGCAAAA
CAGCTGGGATCTTGATAGGGATTTAGTTGAATTGGTAGATAAATTTGAGAAATAACTGCTATTT
TAATGGTGTTAGGTTTTCTGTTCCATGACTGTGGGATGTCTTTGCATTTGCTTAGGTCTTTATT
TCAGTGATGTTTTAGAGATTTCAGTGTGCAAGTCTTGACGTCTTTAATTTTTTTCTAAGTATTT
TACTGATGCCACTTTAATTATATGATTATTTTCTAATTTTTATTTTTGTATTCATCACTGGTGT
TAGAAATAAAATTAATTTTTGTATATTGATTTTGTATATTGTAGCTTTTCTAAAATTCTTTTTT
TGTCACTTAGTATTTTTTGCATATGAGATCATGCATCTGTGAACAGAAATACTTTTACTTCTTC
CTTTCCAGTCTAGATTCCTATTATTTCATTTTCTTGCCTTAGTGTGCTGGCTAGCACCTCCAGT
ACAATATTGGATAGAAGTGACAAGAGCAGACATCTTTTTCTTGTTCCTGATTTTAAGGGGAAAG
CATTCAGTCTTTCACCATTGAGTATGATGTTAGCTAGCTGTGGGTTTGCTTCAAGTTGAGGAAA
TTCTTTTATTAAACCTAGTTTACTGAGTGTTTTTTAATCAAAAAAAAAAAAAGGAGGGGCTGTG
GGTTTTGTTAAATGCTTCTTCTGTATCAAGATTTTCATGTTGTTTTGTCCTTTATTAATATGTT
GTATTAATGGACTTTTGTATATTGAATTAACTTTGCATTCCTGGGATAATCTCAGTTGTCCATG
CTACATAAACCTTGTTCTATGCTGCTGGATTTGGTTTGCTAGTATTTTGTTTAGAATGTTTGCA
TCTATATTCATAAGGAATATTGGTCTGTAGTTTTCATATTATATATCTGTCCAGTTTGGGGATG
AGAGTCATACTCCTGACAGAATGAATTGGGAGCTTTCCCTGTTCTTAAACCTACTGAGGTATTT
TTTAAGTGAACTACTTGGAGACCTTCACCTGTGATCTTCAGATGCTGGGGGACACAGGGATGGT
ACCTGACTCTGAAAAGGCTCTCAGGGTAAGACGTGTCTGAACTGGCCTGTGGCAGTTAACTAAA
GAGTACTCCTACCAATAATGGAGTCAAGATGTATGTTTCCCATAGGCCACGCTGGTCACTGAAA
GAGATGGGCTATGAGCCATCTTGAAAACTACCCTGCACAAATGGACTGCCGGCCTGGAGCCAGG
CACCCTGCAGCTGAGTGCCATATAATGTTGAAGCTGTTGAGGGAGGAGAGGTTGGAAGCTGCCA
GCTTAGAGAATCTCAGCTGTGGTTTCCAGCAGGGTGTATTCCAGGAAAGAAAGTCATTCCCCAC
CCTGGCTCAAAGTAATAATTAACAGTGACTGGGTAAGGCAGGATCTTTTTTTCTAATTCCCTCC
CCTCCAGAAAGGGCAGCAACAGCTCTTGAATGGGAAAGAAGGTGGAGGGAGGAAAAGAAAAACC
AATTGCCCCAACACTGCTGTCTTTAAGCTTCCCACCCAAAGACCTCAGTAAGAGGCAGAATTAT
GTTTTCAATTAAGAGAGAGTTTGGCCTCTTGGTGTTTCAAAAGTGTAGGCTTTTTAATACCTAG
AGAAACTGATGATTTGTTGATTACCAAAAAGGGACCAGAAAAGCTACATAGCCTGAGATTTCAT
CCCCAGAAAAACGAATCCAGAGAGCAGACTTGCAAAAAAAAAAAAAAACCGTTCTGTCTCTGCC
TCTCCCACTACACATACACAGTGAGAAAGAGAGTCTTCATGATTTGGTAAATTAGACCCATGGA
AAGACTCGGGACATTTAGGATGATGATATTTACGATGAGGAAGATTTCTATGGGAAACTTTTTT
TTAACTTGTCTCTAATCCTATTCCAACTGAAGTTTGAATTTTTTTCCTTTCATTAACATTTACT
GGGCAAATACCTATTGAATGTTTGCCATTTTGCAGTGTGCCATAGGTTTCAGGCTGATTCAGAG
CTGCTTTGTTGAATCAAGAAGTCTGTTACAGGGAGACAATCTAGTTAGACCAGGCATAAGTACA
AACCACTAAGGGTAAAAGAAGTATGTGAAAGTACAATGTGGGCAGAGAATTCAGATCCAAAAAT
ACTGCAGATAGGTAGGTGATGAAGGTCAAGCCGAATCTTGACATACAGGATAGGTGTATGTGAG
TGAGTTAGGTGGAAAGTCTAGTATGTCTGAGTGTCAGGCTGTAAGGGATTAAGATGAGATTGGG
AAGGCAGGCAGGTCAGATAATGAGGGCCTTATAGACCATACCAAGGACTTTGGACTTTTATTTT
GCAGACGTTAAGCAAAGACATAATCACTTTTCATTTTATATTTATTACAAACTGAAAGAGCACA
AGTGCACATGTGGTCTTTGCAGTTAAGTTATTGCCATAATTCTAAAAAGGCAGATAATCTGAAG
ATAGAAGGGAGGCCACAGTCCAAGACATGCTAGGGACTCTGATTGATTCAGTGCTCATGCATGG
GAAAGATAAGAGTCAAAGACAACTCCCACATTTCAGGTTTTGAGAGCTGGACGGACAGTGTTGC
TGTTAGGTCTATAGAGAAGAGGAGTGGATTTTGAAGGGAAAATTATTAATAGTACAATTGAATG
ATGTTTTAGTTGTGTCTGAGGGATATCCAGGGAGAGCTGTCCTAAGGCAATTAAAAATATGGGT
CTAGAGAAAAACGTACACAGAAGATAAAAATGTATGACTCCAGCACATAGACCATAGCTGAACC
TGTGGCTAGGAAAATTCTCCTGGAAAAGGCAGAGAGGAGAGAAGGACAAGGATGGAATGCATGG
AAATGCTAGCATTTGAGAGTTATACACGGAACCCACAGTAGAGGCAAATGAGAAAAAGCAGGTT
TGTTTTTGTCTGCTCCAGTAGACAGACACCAAGCTCCATGAGGGCAGAGACTTTGTCTTATTCG
TGGCTGTACCTGCAGTGCATAGAGCAGACTGTACATGTGCTTTGTGGACCCATGCAGTTACTGG
GGTGTCTCAGAAACTTCCCATCCTCCTCCCTACTGTGTCTTTGCATAACCCTGGCTGGGCCTCC
CTTTTCTCAATGTCAACTCTCACTGTAACTTTTTTTTTTTTTTTTTTTTTTTTTTTGAGACGGA
GTCTCGCTCATTGCCCTGGCTGGAGTGCAGTGGCGGAATCTCAGCTCACTGCAAGCTCCGCCTC
CCGGGTTCACGCCATTCTCCTGCCTCAGCTTCCTGAGTAGCTGGGACTACAGGCGCCCGCCACC
ACGCCCGGCTAATTTTTTTGTATTTTTAGTAGAGACGGAGTTTCACCGTGTTAGCCAGAATGGT
CTCAATCTCCTGACCTCATGATCTGCCCACCTCGGCCTTCCAAAGTGCTGGGATTATAGGCGTG
AGCCACCGCTCCCAGCCTCTTACTGTAACTTTTAAGAAGGGCATATATCTACGTAGTGTCATCA
CCATAAAGATAAATCAACCAAGTGAAAATATTTTGAAACTTTGGAAATGTATAAACTCCAAAAT
CATCATCAGTTACATCATCAATTATGTCCCTAAAGATCCCATCACTCTGCCTCACACTGGGGTC
TTGCCTACTTCTCTGTTCCAAAAGGGCACAGTAGTGGTATCAAATCTGTTTCTTGAGGCAAATG
AAGTACCTTGCTCCAAAATAATCACCCACAGCAGGCAGATAACCTCCAAGTGCTTCAAGCTGAG
ATTGAGCAGGAGGATCCCAGCTCATCACAACTGGGTGGTAAATATACACTTGGGCCCAAATGGA
AGGAAATGCATTACCCTACAATGCATACCCAACATGCTTGCATCCAGGCCCCAAATCCAGCAAA
ACAATGCTATTCCCCTTCAACATGTGTTAACGGAACTGAGATATTTTTCCAATTTAAATTGTGT
TCCCCAAAGTCTTCATTATTCTCTAGTGCCTATTACAATCTGTGTTTTTATGTTTACTTCATGA
CTGCCTAGTTCCCTCCTGAAATTTTAAGCCTCGTGAGAGTAAGAACTATCTGTTTTCTTTACTC
CACTGTCCTGTACATACATACTTGTTGAATGAATGTATGAATGCCACCGTGATTGCAAAGTCCA
AGGGAAATGATGTTGATTCACTGCACACTGTACAGGAGGGTGTCAACCAGACTTCCAGATTTCT
ACAGAAATTACCAGTAAAGGGTAGTATAAGAATGGTTCCTGCATGTGAAAAATGATCTTGGATG
GAGCTGTTGTGGCATTCACCTGCTCCTCATACTCTCCTGCCACTGTGCAGGGACAGCACAACCC
CCGTGCTGCTTTCTTGAGTGTAGCTCATTTCAGAAAAGCAGTAAAAGGAAAACCCACTTGTTTG
CTTTATGTTCCTAAGAAGGTGGTGAGTAAATCAATCACGTTATCCTAAGAAGTGACTTGAATTT
TATGAAGGCTCCTTTGTTTCCATTGCACTCCTTTCTACCCATCTCTACTCCCACCTCCACCCCC
ACCCTCAAATCATGCTTATAATTTCCATTATTCAATTAAGGGTGGCTTTTTTCTCATCACAAAC
ATCTGTGTGCTTTAAATAGCTTCTACATAACCGAAGTGGAAGTAGTCATGTAGAACAGCACATG
GCCACTCCAGGGGACATGAGAAGCCTCTGCCTAAAGGTAGTTCTCCCTTCTGTCATCTGATCTC
ACACATGGCACTGCAAACCTGCAGTTATTTAGCCATGTGCTTTGTGACATTGATTTCACTTCCT
TGGGTTATTCTTACCGTCTTTAGGAACTTAAATATTTAAGAAATAAACAAATACTATTCAAAGA
GAGCTGTACCATTATTATATGCATTTAAGTGAAAAACTACCACTTCCCATATATATTTATCTCA
TGACCACCTAATTGATAAAAACATGTTAACTGTTATCTTATATTGCCTCAGGGAAAAAGTCTGA
ATGTTGATTGGAGGCACTGCAGGATAGCCATTCGATACCTGCTTTTGTGACAATAAACTAAAGC
ATTGGATTTTGGTGGATTAGGGATAAAAATGGCTTGACAGATCATCTTGTCCAGCCCTCAGTAT
ATAGATGAGAAGACTTAGGTCTCAAGCTCTGAAGCGCTTTGCTCAGGATCGCTGGCCATTTCAT
ATATGTAACAAGCTCCCACTTCATGCTCATTTCACAGTAGCGTGTCACTTTCAGGTCAGCTTCC
TTCAGTGGGCGTTGTCTTTGCTTAATTAAATGTCAGGAGGTCCCCCCAGAAGGCTTGACTCAGG
GAAGGAGGGCAGACCCTCCCAGTTCTCTCACTGACTAGCTTTCGTTTGTTGGATTGTTTTTACT
GATAAAAAAGTTCAGAAATTAGAGCCAGGTTCCTGTTCACGTTCCCTACCCAAGCCCCTGAAGA
AGCAAATTGGTGAATGTTCCCCAAATTCTTTTACTCTCTAGTATACAGTAAGCAGTACTGCCAA
AAAGTTTCCCAAAGTCATGTTCCTCTTAGATATTTTTCCTTGAAAACTGTTGTCTGGCATGGAG
ATCCAACCTGTGTGGAAAGTTCCAGATGCCCTGGTCCTAGCTTCACCTGCCCTGCTTTTGCTTC
CTGTTCTCACTGCTTGTCTCCAGGTTTCCATTTGAAGGCTTGCAGTGCTACTGTGCCCTTACTG
TTAAATCCTGCCTTGCTTGCCATGTTTTATAGACAAGGAAAGCATTTTCTACCAGAGCTCAGGA
GCAGCCTTGAGGTCAGGGGTAACGTTTTTCATTGGTAAGTATTCAGTAGTTGCTGAATGACAAT
TGGGTGGAAAGGGGAGGGGTGCCTTAGAAAAAATGTCTGTTCTTTGTGGTAATGAAATTGCTTC
CAATTTATTTTCTGATCTCTTTGAGAACTATTTCCTGTCGTCTTCCCGCACCATGCATTCTGAT
TTCTGGGGCATTCAGTGCTCCCTCCCTTCCTTTATTCAAGGGGCTCTTGGAAAAGAAATCTCTG
AAGAAAAAATGTGTGGGCAGTGGGAGACTTCTCATTCTCTCCACCAAATACTTAGCCCTTTCAT
TCATGAGAAACCCCTGCTTTAGGATGGTGTGGATCTGAGTCAGCAGGGCTGGGTTCATTTGTAT
ATACTCCAGGTTGTGGAGGTTGTTCCTGGGTCTTGGCCTTTCATTTTGGGAGTGCTCCACACTT
CTGTCTCCATATACTATGTATTTAAGAAGCTTGCCTTTCGATTTCCTGCCAGTGGCTGGATCTT
GGGGATCACTGCACAATGCTTGTATGCCCCACTGGAGAAGTCTCTCATGGCACCTTTCCCCTCC
CATGGTGGTGGAATGCTGTCCACAGAAAGCTTCTCTTCACTGTTCAGAGACTCTAAAAATAAGA
CCTGAACTGACTTGCTCTGCCTACTGAAAGGGGACCTCCTTATATTCACCATTGCCCTCACACC
CTTTTCCTTGGAGTCTGTCTTTAGGGTCACCCTGGGAGCTGCCTGGGCCTCCACCTTCTTAAGG
AGGCAACTCCTGAGAGCTGGGTAGGGACTGTCTTTGTCCAGACTGTCTGAAGAGCATCTTGTTC
TTTACCTCCCTCTCCTGTTCTTGACTATTGCCTTCACTGCTGCCTCGCTCTCGTCTACCAGGGT
GGTATTATAAAGTATGTCCTTGGAGGTGAGCTGTTTGACCAGCTCCTTGTTGCCTAGAAACCTA
AGAAATTGTCAGAACAATCCGGCATGCATCAGCTCTGACCTTTGGAGCTTTGACTGGCATTATT
TATAAAAGGCTTTCTGCTCTGTTAGCCGAGCATTGGCTTCATATTTCTCCATGGACTCCAGAAA
ACTGGGTTTCTTTTTTGCAAGTTCTCTTGTTTTCAGTTGAGAACGGTATTGAGTAGGACTTCTC
TGTTTCCTTGAGTTTTATGTTCTCTCCAAATATGGAATAGCACTTGCTTCCCTGGGTGCCAACG
CTGGCCGACCAGCTCTGAAAGTTGCTCTGCTTGTGGTTCTTCCCTGAGATGCTAGTTGGGCCTG
GGAATCTTCCATTTCTCTGCCCACCAGGTGACCTGGCAGTCAGTGTTCTGTCAAAGACCTGTGA
GGGTGCTTAGTGAACTGTGGGCACTATGACCACAGGGTACTATTCTGGGTCCCAGTGTTTGTTT
TTAAGAACTCTGTAGGTTGACAGGACATGCTGTCTGGTGGCCAAGTGCTTCTGCCAAGAGTATG
GGGCCTGGGAGACATGTTTCCTGGCCTGCTTATTGCAGGCCTAGCTTCCACCTAAGAGCTATCC
GCTTCCCAAGGGGACATCCACCATGTCTCTGTGATAGGAGCTGAATAGTAGGGGCAGGACTGAA
AGGTCTGTATGTTTGCTTTTCAGTCAGGTACAGGTGTTGGAGGTTTCTTAAATTTGGTTTCTTT
TGATCTCCCTGGCAGGCAGTGATGGGGAAGTTTTTAGTAAGGTTATTAACAGAGATAGAGGATT
TAGAGTTAAAATTTAATAGGGAAATTTTTCTATAGGATGGTTGAAAATGATGCTTTGTATAGTT
TTATTGTATGAACTTTTCAGGGATAGAATTAGTCAATTTGTAGAAATTGGGCCGCCTCTGTTAT
TGTGATATATTAGAACTGTATTGAATGATGAGACTTCCCAAAAGTTTAAAACAAAAAAAGGAGG
CAGAAGAGAACGAGGATATAAATGAGGACACTCTCGCTGAGGACACATCAGCTGTTAGCAGTCT
GTCTCTGTGTACCGAGCTCAAGAACTGTGACTTTGGTTGGAAGTTAGACCTTTTCTATCCAGCT
TCAGACATGGTAACCTTCACATAAGCTGCTGGATTTGTTCTACCAGAAATTTAGTCTGTTGTGT
GACTCCAGCCACTTTGCAACTCAAGATTATTACTTGTTCAGGTTTCTTTCATTAAATAGCTAAC
CATTTAACAACTATTATCTTCTTAGGGTCGACTGATGGTTACCCATACAGTATATACATCATCC
ATATCCTTTCCTCTCCTACTATATCTCACTCCTATTTTCCAGTCCATCCTCATCTCTCCTCAAG
ATCAACAGACCCCAACCTCTTTTACTTAAACGTTGTATTCCTGAAGTAGTCCTGCCCTTTCCTT
CAGCCTGCGAAGCCTTCTCAAAGACTTCTTTTCCATGCATCTAGACTCTGCCTGGTTATTCTCC
TTCCTTCAGAGTTATTTCTTTGTCAAATGCTGCTAATGTATAAAAACACAAAGCTTTGATAGTT
CCTTCTGTCCCAACGGAGCCTTACGCTTGGCTGACATTTAGGGTAAAGGTGGTATTTTTATTAG
TAAAATTAAATGATACAGAAGTGAATTCTAAAAATAGTTACATAAATCTAAGAGAGTTTTCATG
AATTCAGGTTGCCCCAAAGATTTAGGCCATACCTCTTCCATGGAATGGCTGTCATGCAGTTTGG
ATGGTTGACCTCAAAATTATTCCCAGCCCCAGAAGTGGAGTAGGTATATTAGTCTAAGGCAGTC
AGGGTAATGCTATCTTCCATGCCACAGATTGGCTTGGAATGGGGATAACCAAAAGCACTTTGGG
CCTGTGAGATTTGACAGAGTGTTTACTGTGAGCCTAGGGAAAGAACATGCCTGCTCTCATGTGA
GAGTATGAAGAAACACTGCCTAAGCAAGGAATCATGTGGCTCCAGTTTTTACTGTCAGCCTTTC
TACCACCAGGGAGACCAGCCTTGAGATGATGCAGGTGCATGGACAGCTGTGTGGAGGGATAGAA
GAATCCAGTTTTTAAACCAGATGGCTGAGCTATTTCATTAAGTCACCCCAGAAGTCTTCAACTA
ACAGTATCTTAACTGGTGAACTCCTGTGACTGATACAGGAAAATTTTACTTCGTAACAAAGTAT
GATAATTCCTTTGTGCAGGAAGGAGGTTTTGTGGAAATGATGTTGATTCACTGCACACTGTACA
GGAGGGTGTCTCTTTGACACCACCAACCTTAAATAGTAGGAAAGCCATCACAGTGGGTTGGGTG
GTTGGATTGCTTTATTGCAACTAATAAAGGGTCAGTTGTGCAGTTGTTAACTATCTTTCACTTC
CCTCAGAACTGTATCTCCTAGCCCAGTGCCCTATAGGTCAAAAATGCCAGTTTGTTGTACAGTC
AGCAGAGCCCAGTACTATGCTTTAAAACATTCGGGGATAATCATCAAATATTTAATCAGAACAG
ATGCTGCCTGTAAACAACCACTGCAGCCAAGAACAGTCTGCATCTGAAACACTGCCTGCAACTA
TAAATATGCCCATATGCTTGAACAATTTTAGTGTTGTAAAAATCTTGTTTTTATAAGCAATTAG
TTTCATCTCCTATTATATGAGGATTAACTTTGCAGTGATGTACTGATGTTAAAAGAGAAAAGAA
TCAAAAAAGCCTTGGTTGTGGTGAAACCCAGCAAAATGTGGGTCTGCACCAAGTTCCAGATGAA
TATCAAACAGCACTGTCCCAGTTGGATTAAGGTGATGACACCCCTACCATGGCTACAGTACCAG
GTCATTTGATTTTCCCCAGGGTCATAGGAGTGTTAGCCCAGGTGATCAGCTCAACACCCCCTGA
GATCCATGAGGTATATACTTTGAAATTTCAATTCCATTACTACTGACTGCATCTCAATCCTATG
TTAATACTTTCTTGAGTAATATTCTGTTGACCTAGTTATCCACGTTGGAAACTTCAAAGGCATC
ATCTTCAGCTCTTTGTTTCTGACTCCCTATCCAGTTCTCCAGTTCTGTATACACCATCCCAGAA
ATGGTCCTCGCATCCAGTTAATCTCTGACATGGACTAATAGGCCTTTCTACAGCTTCAGTGTAT
TCTCTTCTCTGCTTCTGGAACCCAGGATTTAGGTGCTACAGTCATACTATCTTTGACATTCCAT
CTCCCTGGAGAATTTGGAAATGGGGAAATAACATTACTCTTAACAAAGCAAGATTAACAGTCCT
GTAACCAGACCCAAGGCACTGACCCTTCTGTGAAAGCCTATATCCAACCAGCTACAGGAACCTC
TACAGAGAGACTAAAAAAGGCCAGCCCTGGAGGAGCCAGTGGTCAGGAGAACATAGGCATAAAG
CCTCCTAAGGATGTAGCCCATCAACCCTGGGAGTGGGTTCCTATAAGGAATAGGCACTTCGGGC
AAATATGGCCCGATGACACCAGCCACTGGGATCTTCTGACTCTCTCCTGTATAATGTGAGTTCA
TTTGTTTATTGTGTTTAAAGTGAATTCTGCCTCCCTAAAGGCTGAGAACTTCCTTTGGCTCAGT
GTTCACTTCGTTTGGCCTCTGACTTCCAACTGATGCATCAAGCCAATGCGCAAGAAAAGTTAAT
AAATAAAAATAAGATTTATTACAGAGCCAGCTGGGCACTCATACATACATGGAGGAGTCCCTGT
GTCATTGGAACACTGTAGCTTTTCTCTACAACAGTCCCAGGCTAGGCAGCTCTTTGTAGGCCCA
AGGGAATTGATGCATAAGTAAGGCATGGTCTTCCTTGACTACACATCTTGTATACAAGGTATTA
AGGCCCTTGGCCACCTGACTTCGGCCCATTTTTTCAATCTCAACTCTACCCTAAATTCCCCACA
CAGGTCATTCATTCTCCAATGAGGTTATTCTCCTGATCTCTGTGTGGGTTCTTATACATAATGG
AATTGTGTGTTTTTCTCCTGTTTAAAGTTTGGAACATCATGTTAGCCATCATTTTATTCTCTTT
GCTTTCACTGTATTGCACTCACATACTGTATGTGGAATCAGTGCTACCCATTAGACCCATGGCT
CATCAGTGGGAAATGGTGTCAGATCTCACAGAGGGCAGCTTCATGGGGCTCTAGGTTCTTCAGG
CTTCCTTCACAGCCTAAGCTCCATATTTAAATCCAGCATTCAACCCTAATCACTGCTGTTCACT
AGCATCTGCTACATATTCTTGCTGACAGACACTGTGTTTGCTCTTGAAAGATAACAAAATGCTT
GAAGCTTCCCTCCAACGAGTGATAATTCACTGAAGCTCAGAGGCAGCAGGAGCTCCAGTCTGCT
CTGCTGCTCTGAAATTGTGGAAAGTAGGAAAGGTGTCCAGAGGTCATATGGCCCAAGTGATGAC
ATATCTGAAGCACAGAGAGGGGATGGGCTAAGAGTCACCTGTATCCAAAATTCAAGTTAACACC
TATCAAGTTGTGAAAAGTTACTTTGGGACATCCCAAAGTTACACAGAGAGCTAGAGCTAGGACT
CAGACCTGGAACACCAGACTTCAGTCTCTCTGAACTGCACTGGGTTGTCTCTGCTCAAGCCTTA
TTGGAGAGAGAACTGACAAAAGGCAAGGCTAAGGAGTGCTCTTTGTGGATGCAGCAGGAGAAGA
AACACCCTTCCAATGAGGGGTCCACACAGAGTTCAAATGTCCCCTTTGTTCATACTGAACGTGG
GTATGGTTTTCCTCTTTTGCTGTCTGCACAGGTGGCATAGAAGGCAGCACCTGATCCTTCAGGT
CTGGGGGAGGAGTCCCCAGATAATGCATAGATCTTTTAAACTCTTCACGTCAAACTGACATCTG
CTTATGTAGCTCAGCTCAATGTGTTAGTCATTTTGTTTCGTTTATTATCAAACACATCTTCTGA
AAAATGAGAAGAGGAAGGGAAAAAGGAGTCTGGGAAAGGACCACACTTTCTACCACCCCTGTAA
ACAGTGAGCTTTACTCTGCAATACACCAAGTGGAAGGAGACAGGCTATGAGATGGGCACAGTAA
AGAGCCACCTAAGGGAAGTGGGCAGAACTCCAGAAGACTTCCTGAGGAGGCATTTATTCTTTAT
GAATCTTTGAAATGTTTTTTTAAATCATCTTTGATTTGACTTTTGCTCTAAAAGTATCACAAGG
TAAGACTCAGCTGTGAAACTAAGCACATCCGATAGTCAAACTATGAAAACTTTTACACGCTAGT
GGGGCACTGTGACACCACTGATTTCCTTAAACTGAGCTCCTTTCAAGGTGTTTTGACCTAACTG
GCCTTTGGGTCGGATATAGCTTTTACATACTTTTGGTAGGTAACTACGGGTTCCTATTTGTCAG
GGAAAGTGGGTAAGAGTTAGCTATTCCCCACAACCTTGATAGCAGCTTATAAAGAGAGCCAGTG
TCAAATCAATTTGTGCCACAGGCATTATTTTAAAAGCATTTCTTAGAAAGCACACACAGGAGTG
TTCAGGCAAGAGGTACTGTAAGCCCTTTGCTTTTTCGGGGCTTACAACCTCTCAATTTAAATGA
AAGGTTTATTACACAAGGTATTGGTAACAGTGACCAGCAATAGGCTCTGCTGTGTTACTACTGG
CATCAGTCAAAACATGTTCTGACCTCGTTTCTGTATATGTTTATATGTTTAGAGGTCAAGCCCT
GTGTGTCCTTTCTGAGGGCCATGTTAAAGGCCTCCTCAGATTCCTATACTCTATTCTTGCAAAG
CTGGAGAGGTTCTTTAGAAGTATTTTAGCCGGGATACGTATATCGAATACCCTCAACTTCACCA
ATGCAAGCCAGCTTCTAACATACTAATTAGCAAGCCACTGATTTTATCAATAAAAACCCATTTC
TAACATTTACTGTGGAATTAGCAAAGTTTCTGGTGTACATTGGATATGAAAGCTCCAGTAACAG
ACTCAGAAGAAATGGAGACCTGTGAAATGACAAATAATTTAGACTAATCCTCTTAAGTTCAACA
AAGTACAAGAAAATATGGATAGAAAAATGAAATTTGGAAAACAACATCTGAACAGAATGAGAAG
TTTAACAAATATAAACAAAAACAATAGAAATTCTAAAGATAAAGAATACAGTAACTGAACTGAA
AATCTAAGTAGAACGCTTCAACAGCAGACTTGATCAAGCAGAAGAATCAATGAGCTCAAAGGTA
AGACATTTGAAATTATCCAATGAGAAACAAAAACAAAAAAGAATGCCTATAAGAATTACAGGAC
ACATCAAGCTAGCTAACTTTTGCATAATAGGAGGTCCTTAAGGAGAAAAGAAAGAAAAAGGCCT
AGAAAGCATATTTAAGGAAGTAATAGCTGAAAAATTCCCAAATCTGAGAAAAGATGACAAAATT
CAGGAACAGGAAGCTGAGGTCACCGATCAGATTCAACCTAAAGAGTTCACCAAGACACATCACA
ACCAAGTTATCAAAAATCAAAGACAAAGAATACTGAAAGCAGCAAAGATAAGAAACATCACATT
CAAGGTAGCCCCAATACAGATTCAGTGGCTTTATCAGCAAAAGCCTTACAGGCCAGTAGAGAGT
GGGATGGTATATTCAAAGTGCTGAAGGAAACAAAGCTGTTAATTAAGAATACTTTACTTGGCAA
AGGGAGAAATAAACACTTTTCCAGATAAACAATGCCTAAGGGAGTTTGTCACTACTAGGCCTGC
TCTACTGGAATTACTAAAAGGGAGTTCTTTAGGCTTAAATAAAAGACTGCTAGTTAATGAGAAA
CATAAAAGTAAAAAACTAAATGGTATAAGTAATTCATAGTCAGAATTCTCTAGTATTGTAAAGG
TGGCAGGTAAAGCAATGTTATTCCTATTAGGAGGTTTAAAAGATGAAACTATTAAAAACAACTG
TAGCTACAATAAGTTGTAGCTACAAGAAGTAATTGTAACTGTAGCTACAATAAGAGATACAAAT
TTAAAAAAGTGTAAACTTTGACATCAAAATCACGAAAGGCGGGGGGGTTGTGAAAGCGTAGAGT
TTTTGTATGCAACTAAAGGCAAGTTTTTATCGCTTAAAGTAGCCTAAGGATAAAATGTTTTAAG
TTAGCCTTACAGCAAATACAATGGTAATCGCATATACTATAAAAGAAAGGATTCAAAGCACACC
ACCACAGAAAACCACCAAACCACAAAGGAAGGTAGCAATAGGGAAAAAAGAAACAAGGGACCTA
CAAAACAATCAGAAAACAAATTACAAAATGACACTAGCAGTTTCTTACCAATAGTTACCTTGAA
TGTAAACGGATTAGATTCTCCAACAAAAAGACATAGAATGACAGAATGGATTTTTTTTTTTTTT
TAAGTCACAACCACATACTGCCTATAAGAGACTCACTTTTTTTTCTTTTTCTTTTTCTTTTTTT
GAGACGGAGTTTTGCTATTGTCAAGGCTGGAGTGCAATGGCATGATCTCAGCTCACCGCAACCT
CCACCTCCCGGGTTTAAGCAATTCTCCTGCCTCAGCCTCTCGAGTAGCTGGGATTACAAGGGCC
CACAACCACGCCTGGCTAATTTTGTATTTTTAATAGAGACAGGTTTTCTCCATGTTGGTCAGGC
TGGTCTCTAACTCCCAACCTCTGGTGATCCCGCCGCCTTGGCCTCCCAAAGTGCTGGGATTACA
GGAGTGAGCCACTGCGCCCGGCAAGAGATTCACTTTTCTACTAAACATACACATAGACTGAAAG
TGAAGGGATGGAAAAAGATATTCTATATAAATGGAAATTGAGAGCAGGGGTAGCTATACTTCTA
TCAGACAAAATAGACTTTAAGTCAATACTATAAAAAGAAAGAAGTTTGTTATATAATGATAAAG
GGATTAATTCACCAAGAAGACATAACAATTGTAAAAGTATATGCACCCAACATTGACCCACCTA
AATACATAAAGCAATTTTTAAATGATGTGAAGAGGGAGATAGGCTGCAATACTATAATACGGGA
CCTCAATACTCTACTTTTCACCATGGGCAGATTGATCACCTAGACAGAAAATCAATAAAGAAAC
AATGGATTTCAATTACACTTTAGACCAAACGGACTTAACAGGCATATATGAAACATTCCATCCA
GAAGCAGCAAAATACATTTTGTTCTCAAATGCCCATGGAACATTCTCCAGGATATATGTTAGTC
CACAAAACTAATCTTAAGTTTAAGTGGACAGAAATCATATCAAATCCCTTTTCTGATTACAATG
GCGTGAAACTAGAAATCAAGAACAAGAATCAATTCTGGTGGAAAATACAGCAATATGTGGAAAT
TAAACAATATGCTCCCAAACAACTAATAGTTCACAGAAGAAGTCAAAAAGTAAATAAAAAATTA
CCTTGAGACAAATGAAAATGGAAACAAAACTTATGTGATGCAGCAAAATCAGTCCTAATGGAAA
TTTATAGCGATACAAACCTACGCCAATAAAGAAGAAGGATCACAATCTCACTTTTCACCTAAAG
GAACTAGGAAAAGAACAGACTAAGCCCGAAAGTTAGCATAAAGAAGTAAGTAACAAAGATCACA
GTTGAAATAAAGACTGGAAAAAAATGAACAAAACTAAGAGTTGCTAAGAGTTGGTTTTTTTTTT
TTTTGAAAGATAAAATCAACAAATTCTTACCTAGACTAGGAGAAAAAAAGAGTACTCAAATAAG
ATCAGAAATGAAAGAGGAGACATTTACTACTGATATCACCAAAATACAAAGGATTATAAGAAAC
TACTATGAAGTTATGCACCATCAAATTGGATGACCTAAAGAAATGAATAAATTCCTAGACATAT
ACAACCTACAAAGAGTGAATCATGAAGAAACAAATCTGAATAAGCCAATAACAAGAAAAGAGAT
TGAGCCAGTAATAAAAAGTATCCCACCAAAGGAAAGCCCAGGATTAGGCAGCTTCACTACTGAA
TTTTACCAGACATTTAAAGAACTAATAAAAATTCTCAAACTTTTCCAAAACAATGAAGCAGAGG
GAATACTTCTAAATTCATTTTACAAGGGTAGTGTTACACTGTTATTAAAGCCAGAGAAAGACAC
TTTAAGGACAGAAACTTACAAAGTAATATCCCTGATGAACATGGATGCAAAAATCCTCAACAAT
ATATTAGCAAACTGGATTCACGAATACAAAGAATGATTCACCATGATGAAGTGGGATTTATCCT
TAGGATGCAAGGGTGGTTCAACATATGCACATCAAAAAGTGTGATATACCACATAACAAAATGG
AGGGTGTAAATCAAATCATATGATCATCTCAATGGATGCAGAAAAAGCATTTGACAAAATTCAA
CCTGGTTTCATGGTAAAAACTCTCAACAGATTAGGTATGGAGGCCATGTACGACCCTCATCACT
TTTTGACATAGTACTAGAAGTCCTAACCAGAGCAGTTGGACAAGAGAAAGAAAAAAAAGGCATC
CTAATAGGAAAGGAATAGGTTAAATCATCTGTAATTACTGATGACATAACCATATACAAAAAAC
TGTAATGTCTCCACCAGAACAAAACTAAAACCTGGTAGAACTGATACACAAACTCAGTAAAGTT
CCGTAATACAAAATCATCTTACAGAAATCAGTAGTGTTTCCATCTACTATTAATGAGCTATCTG
AAAAGGAAATTATGAAAATCTCATAATAGCAACAAATATTAAATACTGAGGTGTAAATTTAAGG
AGGTAAAAGATCTGCATACTGAAAAGTATAAAAGGACAGGGTCTTCAATAAATGGTGTTGGGAA
AACTGGATATCCACATGCAAAAGAATTAAATTAGACCCTTATTTACACCATATAAAAAAATAGA
TTTAAAACTTAACATATAAGACCTAAAACTGTACACCTACTGAAGAAAACATAGGGGGAAATCT
CCACAGCATTGGTCTTGGGAATGATTTTTTTGGATATGACTGCAAAAGCATGGCAAAAATAGAC
AAATGG

What is claimed is:

1. A polynucleotide comprising a nucleotide sequence encoding a ribozyme, wherein the ribozyme is capable of inhibiting the silencing of the paternal UBE3A gene, and wherein the nucleotide sequence encoding the ribozyme consists of SEQ ID NO: 3 or SEQ ID NO: 4.

2. The polynucleotide of claim 1, wherein the ribozyme increases the expression of the paternal UBE3A gene.

3. The polynucleotide of claim 1, wherein the paternal UBE3A comprises a nucleotide sequence of SEQ ID NO: 509.

4. The polynucleotide of claim 1, wherein the silencing of the paternal UBE3A is by the RNA transcript encoded by SEQ ID NO: 1.

5. An expression vector comprising the polynucleotide of claim 1 and a promoter.

6. The expression vector of claim 5, wherein the promoter is a U6 promoter.

7. The expression vector of claim 5, wherein the polynucleotide is a DNA polynucleotide.

8. The expression vector of claim 5, wherein the expression vector is an adeno-associated viral (AAV) vector or a lentiviral vector.

9. A viral particle comprising the polynucleotide of claim 1.

10. The viral particle of claim 9, wherein the particle is an AAV particle or a lentiviral particle.

11. The viral particle of claim 10, wherein the particle is an AAV particle with specific neurotropism.

12. The viral particle of claim 10, wherein the particle is an AAV9 or an AAV10 particle.

13. A pharmaceutical composition comprising the polynucleotide of claim 1 and a pharmaceutically acceptable carrier.

14. A method of treating Angelman's syndrome comprising administering to a patient in need thereof a therapeutically effective amount of the pharmaceutical composition of claim 13.

15. A method of activating or increasing expression of paternal UBE3A gene expression comprising administering to a patient in need thereof, a therapeutically effective amount of the pharmaceutical composition of claim 13.

16. A method of inhibiting the silencing of paternal UBE3A gene by the RNA antisense transcript encoded by SEQ ID NO: 1 comprising administering to a patient in need thereof, an amount of the pharmaceutical composition of claim 13 effective to cut the RNA antisense transcript encoded by SEQ ID NO: 1.

17. A method of treating Angelman's syndrome comprising administering to a patient in need thereof, a therapeutically effective amount of the polynucleotide of claim 1.

18. The method of claim 17, wherein the polynucleotide of claim 1 is included in a viral particle.

19. The method of claim 17, wherein the polynucleotide is included within an expression vector.

20. The method of claim 17, wherein the polynucleotide is included within a pharmaceutical composition that is administered to the brain of the patient.

21. The method of claim 17, wherein the polynucleotide is included within a pharmaceutical composition that is administered to neurons of the patient.

22. The method of claim 17, wherein the patient is a human.

23. A method of activating or increasing expression of paternal UBE3A gene expression comprising administering to a patient in need thereof, a therapeutically effective amount of the viral particle of claim 9.

24. A method of inhibiting the silencing of paternal UBE3A gene by the RNA antisense transcript encoded by SEQ ID NO: 1 comprising administering to a patient in need thereof, an amount of the viral particle of claim 9 effective to cut the RNA antisense transcript encoded by SEQ ID NO: 1.

25. The method of any of claims 14, 15, 17, 21, or 23 wherein the ribozyme reduces the levels of the RNA antisense transcript encoded by SEQ ID NO: 1.

26. A ribozyme encoded by the polynucleotide of claim 1.

27. A ribozyme encoded by the expression vector of claim 5.

28. A ribozyme encoded by the viral particle of claim 9.

29. A polynucleotide encoding a ribozyme, wherein the polynucleotide encoding the ribozyme comprises SEQ ID NO: 3 or SEQ ID NO: 4.

30. A ribozyme encoded by the polynucleotide of claim 29.


SEQUENCE LISTING (509 sequences)

SEQ ID NO: 1 - DNA, 68641 bp, Homo sapiens
  source 1..68641: mol_type = genomic DNA
tgagatgacctaaacaactgtggagaatcattgatatatttccttttttcactgttcatgttgggtgaaaataatcttgtagtgaaattcacatgttctaaatattgtttttttacatctttatctggcacattcataacatagatgtttctatacatattagtactgtaatcataccatatattattctgttaccccacttactccttaaacttttagttaattaaagagtttttataaagtcccccaatagattttttttttttgagacatagtctcactctgttgcccaggctggagtgcagtggcgtgattttggctcactgcaacctccccatcctgggttcaagcaattctcctgcctcagcctgccaagtagctgggattacaggtgcctgccaccacgcccgactaatttttgtattttcagtagtgacagggtttcaccatcttggccaggctggtcttgaactcctgacctcgtgatccacccgccttggcctctcagagtgctaggattacaggcttgagccactgcacccggccagattttaatctaattttattagaacaattcagtcatatgttttttcatgctatgtatatgagagttccattattcagatactaaacaaatgtctactgtacatttactgttctcactgatgatgcattagataaccatgcacaaaataagcctggctgtggaaacgcttatttgttgggagggtgcttgtttggatcgatgatgagaataattgtctgaggatgctgagggactcattccagatgtcaatctgaggtccagatgtgcggccctccaataggacaaataagactctcagagcctggctctatttggggatccctcagtgacaacatagtacccctgtgagcgtgccttttctatctcttcgaagagggcagtggcatcctgtcttatgagtcagtgtgcactttagtgtgcctagtgacccaagacttgctttaattgtagatagatacttacatataggaaatatttcttaagtaacaaatgaaaaactttagaagattgaattaagggtcaagcaactgtgatatgtctgaaaatctcattagtgttgtgctgaaagaaggaaatatggcatgcctctattaaataatgacagtggaaccaagtttattgctttgttatttttactgtggagtattttctaagattatttttgctttttttttctttcatgttttgctgagatagaaggcctggaatctgatcctccacttcagagaacaggggtgagtagctaagccattatcttttgaaattcatatgtcatgtgctctttgctaggtctttaggtcgttttgtacatcttttcagaagcttattggaggacattttcatgatatgtccttttcctcattgagaccctcaccatgtcacctacactattgaatccttatcatttctcttttaattttaactctcttttgcttttatggaaaaatgtagaatttaagagaatttttggcaatttcatattggatcaaaatgtattgtagtgaaatccaggtgtgccaaaatattaacagattttccccatctgtttaattattggggtttcagaatagagactccatggttcataatatctttgtggtcatactacattatatttctgcttctaatttaattattaaatattgacttgaattagtcttttcctcattgttgcaacaaggtaagttatataggaaattttcttctcttgatggcatgtctgagataatcatagatataagacacctggctggtttctagaatgcatgtaatttttatttcttgatctgtgtgttgagtacttgctgtgattgcatcatgcaaatacattgagctttacaattttagtgtatgcacttttctacatgtatattattcttcgatagaaagtaaaaaaaacttatcgaactagtcaaaatattggttaatacataaaaaaagtctcaagtagattgtgtattacatggtgcttgttgattgatgccctcataatagatcaagtgggttctctctttagcacagggctttttagcaaatcatgtcatgagtagttactcaagtatttttattttaacacatttatattttttctatgtatattcttaaattctcttatacttttttctctgttataaaaacatgctgaacaatctcaagtcttaaggattgcagtattgtccccacatattcatgtattttggtactcaattctttatactttctttgacagatcacttgaactggcacatgtctcttgttttgcagagagggaattaatgtgataccttcatgcttttctattctatgtgctacataattgaatatacaagcaaatatagttgttaagatttagtgtgattatttctacaccacatgcaaagaagtttctcatagatcttaatagaggcccacatgcattgtacagtttagaatttggggaaatattgatgaagttgggtaaagtataaagccaaaagtcagaacagtgaactccttgcttaaggatttccttggagattacttagtcaatacacaactgataaatttaagtgcttttcaccttttgagttctcgacatactaaagctaaaatgtgtttcaacttttaatcctgcttccctgattttcccttttttagtctgagatcaaagagtttcagccataaattactgccaagagtaatcacttcattttaagaaagcttaacaatatagaagaatataaaattatttatgacagatgtatttttaaccttttccccatgctttccagaggaaatatgtttaatcatctgccctatattagggaaaaactttctatgctaatacaagtatctatcaatccatttatctttctatataagatgtattgatcataaccaattaactttactgtaaatgagctttagatttgacattttggtagtaatatatgttgtacaatctcctgaggtcctataggtcttgaggtctctatgtcaaaaactatagatgtgccagtgtcctcagtgaatgtgaaggacaccacattttccttagccatttcttgttttcagaataatggttatcaacattttgctactgcaagataccatacatttataatcggaatatgccagtttttatgcactcatgcctctgtttctgtagagcattcccagaatgagtaatgcttgaaaattaggtccatgtgatttctttaatgagttatagtcaaatcatgaaatattcaggttaccattatttcagtaatgtattagaatgtcaaggtaaagttatctacattgtatatatacacacaacatagatataatttatacaacatctatatttatacaacagatatatatttatgtatatatttatacaacataaaatatattttattatttaaacataaaatatattttattatttaatatagattcttaagtgataaatatgtttaatattattaaaataggttaaaataggttatagttagtacagtgaaaattggcagcccttttacaaaacatatgccataactatagcatttatcactgacagtcataccaggatagtcttttatttccaatcacttaaatattcctaattgcaaaagaaatttgaagactaaaattcagaagttttgaaagagccattgcctgggtaaactatacaggtttcagttttatttataataattatgaggccaggcgcagtggctcacacctgtaatcccaacactttgggaggccgaggcgggtggatcacgaggtcaggagatcgagaccatcctggctaacacggtgaaaccccatctctactaaaaatacaaaaaattagccgggcgtggtggcgggcgcctgtagtcccagctactcgggaggctgaggcaggagaatggcgtgaacctggtaggcggagcttgcagtgagccgagatcgcgccactgccctccagcctgggagacagtgcgagactccgtctcaaaaaaaaaaattatgtatatatttataaattaatacttaataaattaataacttgtgataggcaatgcaaagatgacagtaaaaggacaaaattgattagattgataaagtcctgttaacatgaagaaattgaccaggataccatccacactataaagttagagaaatttatcaggacaattccctaaaatactcttctcaattttaacattgtaacaggaatttttaaaattttggtattatgtgtgtttccttccagataatttgaacagattcatatttggtatttttaaaagccatatctttgtccttagtgctggcaatgtattcttgagaatgaacaaataagagatacgtaaaagcataagagaaggtatcaggttgaagtagtcaatcagttatacagaacacaaagaattttatcttgtataatgtttatatagctttatagaagtgtgctgaaagggctataaaacatggacattattatctcattgaaaggtccaatacgtactgaaatacatgcttttattttgaaccaaccaccctataaacgttgtatggcttatttagatgagagcccaggttgtgtgtgtctgtgtgtgtgtgtgtgtgtgtgtgtgtgtgtgtgtgtgtgtgtgtacctgacagggaagcaagaacatcgagttgccaatgcactctgtctatggttagaatcatgctgaaaacatggctcccccagttctggaatgagcccacagatcaagcattccccaaagacatagcaggctcaaatccctgtgtacacaatattttatgattatcttatgtcagtactttcaaagtatacagtttgtgtgaagacaaatccaatgtcatttttcttggctagcctatatgtgtggtaaatccattatttacttacttgcttcctgaaaattacaattagattaacaaactgcagcaaagtgggcatgatgagatagagattgaagtgtaagcttatgttaatgatgcccttggtttggataaacacatctaagagaaaaatggaaaaacacacatggcagggaagccttgatagagccaaaatataggattgtatgtagtaatgcaatccatagatgagcatttggcagtaatattatttttcagatatggataaaaattgcttaggagagtaaagagagacaaagttgaaagcaggtttatagtaggtgttgttttagtgttgatccctttttgctccaataatcaaagtgataaatattgaaaattgattcatgcagcattacttactccattctaatttttatatatgtcaaaagtgccatctcccaaactgtgctatccccttcaggagaagagactctgctgaagtttataaggttgacatattgccagcttcaataatgtaaagatgaagtgtatactgaattcttaatgcaaataacaactctattggaaagtaacccagttatagaagtgctaatttgtcaggagctgccttaccaagatcatgatgagtacagttatctcaggattctgaaagattgttttccgatttcaactagtctagctgaatgttccttgatagaaagagaggacttttagaattggttcaatatgatgacctcctgaattatctcacatagcccgtttgtacatgcctttcttttctctcagaaaatggcactatcataatagctttcttacacagacttcaccttagggttttacattaagggaggggtctggtgtttcatttattttgaagtatttgttgttgattgtgtacagtgcttgagtaaaaaattgaatatagaaacatctagaatatttttttaaaggatcagtgtttataaagtgaattattagtgtcaataatgttgggaaagttttaagagaatataggaaacttgaacattacacaactacaatgggaccaaattgtggggtctcattatagttaatatttatgtatttttttccaattgatttgtgtgctttttttctgcatgtttttggcagatagaatggctataacaagtaacagcatgtcaggtaataaaaataagcagagccctattcctttaaaaatcttcactgatgggagggccataaaataagtcttaatacatttaaagaattaaattcatgtaaaccatgttaatttaattccacaatgatattgaattagaaataagaggaatatctcttgaacatctcctaaatgtttggaaatttaaattagcatttctgacctatttattggttaaaaaagatacaaagaaaggaaaattgaaaagtcttttgaactgaataaaaataaaaatatagaatctaaaactttatgggatactgacaaaacaggatatagggaataatttatagcactgaaatgcctatattagaaaagaaaaaaggttttaaatcagtaaatttgtattttaccttaagaaacttagaaaagaacaaattaacccagacttaagtaaaataaaggcactaataaagataagagcagaaatcaatgaaatataaaacaacaaaacacagagaaaaattgagaaaatttaaaaatagcctagtgagaagatattgataaacttgtaaccagaccaatttaagaaaaaaagtcaaaacacaaataccaatatttgaaaatgtaggagggcaaatcattacagattctatgaatactaaaatgataataaggaaaaattatttaaaaggggcatgtcagccaggcatggtggcttacccctgtaatcccagcactttggcaggccgaggtgggaggattgctagagctcaggcattcgagaccagcctgggcaacatgttgaaaccttgtctacacaaaaagtacaaaaattagctgggtgtggtgttgcacacttgtagtcccagtcacttgggaggctgaggcgagaggatcacttgagcccaggaggttgaggctgcagtgagccatgtttgtaccactgccctccagcctgggtgacaaagtaagaccctatgtaaaaaaaaaaaaatgtatgccaacatttttcaataacttaaatgaaatggaaaaattccttgaaagacacgaactacaaaaactcagtgaacaagtaaataacctgaatagccctgtatcaagtaaattgaatttgtagttaaaagccttccaacagagaaaacttcaggtacctatagcttcatatgaaatgaaaaaaaaaataccaatcctctacaagattccagaacatttaaaagaagggaatatttcccaacttattccatttggacagcaatacccaggtaagaaaaagagacacagaaatttaaaaagaagaatatacattattccttaggaacataaatgcaaagaaatctaatcaaaattttggcaaatgaaatgtagaaatactttatgaccaagtgagagttacccaaagaatttaaggttggttttatatgtaaagatcaaccaatataggaaaatcacttctggaaagtcagagtaagaaactccaaaaatctactcctccataaaaccaataacagccttgatagaaatagttgaaattaattttccaaaactttggaaattaaccaaaggcttacaaaattccagagaacattaattcaagaaaaatggctgaatcagtaagaacagccagctttgtggcattttaatatgaccccttcccatgcttttctccctagtgctgagatagtcttaaaaattagcaggatagcaaccactggagaagaaaggtttggaaatttcccaaaaagttccatccccatagaattatcactatttgacctctaaagcccaatctataggatttatattcatttggactgactcagagctcactcagtaaggaaatctcaatttcaaggtattggtcaaaaagaatccatggcaattgttgactatcacaactgcctgaagtcttggtaacagttgggataaacaagaagctgatcaaaaactgaaaactaaaatcttgggaatgagatatctacaggatgcttcaaaaagctttgatacattcctgtttatctagaaagctacatgcaggctgggtgcagtggctcacacctgtaatcccagcactttgggaggccgaggcgtgcggatcatgaggtcaggagtttgaggccagcctgaccaacatggtctctactaaaaacacaaaaattagccaggcgtggtggcgtgcatctgtaatcctagctactcaggaggctgagacaggagaatcgcttgaacccgggaggcggaggttgcagtgagccaagattgtgccactgcactccagcctgggcgacagagcaaggctctgtctcaaaaaaaaaaaaaaaaaaagccacatgcatgtaattgtttacctctggctttccttttatgctctgggcaagctaaggaagagttgtgaactacctaagtgctgaatgggaaccataacacacacacacacacacacacacacacacacagcaccttagtaaagggtgagaggcatgttagttagaagcattaaaggaaatctctttctagtcattatctgtgcactaacctaactgagcagagacttcagtatccacatactacagggcatataaactttacagaattagtccaggaaaatcatatctaaaaaaaaaaaaaagcagtaacaaaaataaactctgggaaaggggagaatatgatttaaagagttgccacattatacataatatgtctagtgttcaacaaaaaattacgagacatgcaaagaaatagaaaaatatggcacaaagaggataagatgaagtcagtgaaactatcctcgaggaagaccagatgttggtcttactagacacagacattgaaccagctattaaaaatacgtacacagaactaagaaaaacatgtcaaaagggttaaagaaaggtataaaaatagtgtcttaccaaatatagactaccaataaagagatagaaattataagaaaagacaacatgaaaaaatataaagcaaaaaaattagacaattgaaacaagagggcctctattcgcagatttgagcaggcagaagaaagaatcagtgaacttgaagatatgtcaactgagattatccagtctgagcaacaaaggtgggaaaaaatgaagaaaactgagcaacaaagaactgtagaacagcatctctcataccaatggatacataaactggagccctagaaggatgaaaaaaggagaaggaaagaaaacttcccaaattttaagaaaaacattaatttatatacccgagatgaccaataaaatccaattaagataatctcaaagagaccaacacctatacacatcatagtcagcgtgtcaaaagacaaacataaggagagaattcttgaagatagtaagaaaaaaattattcataacatacacaccatcctcaataagtctgacaattgacttctcactgtaaaccatgcaggctaaaaaggcaatgtacataaccaaagtgatgaaataaaaaccttcaaccaatcattctagatccaacaaaactattgttcaaaaagaagaaatgaagacattcctaaacaaaatctcagagaaatgttctctataagacttgtcctaatagaaatgctaaaggaactccttcggtctgaaatagaaaggcactggagagtaaatcaaatccacgagaagaaataaagagaaccagtataagtaactacatgtgtaagtttaaaacaaagtataaatttattttgtttgtaacatttgtcttttcctatttgatttaaaatataatctcaattataaacttgtgttgatggtattatataaagatgtaattttgggttatagcaccaaaatggcagaataggaattttctgtaggtgtttcccacataagtatcaattttgacaaccatccatgggcaagagtaccttgtgggagttcaggagttgacagtaaaacttcagcacaccagaggagtaaagaaatctaagaatagattcattggaaagggtataaacagtttcactttacctgcatcaccaacccccaaaagtggcacagctcagtaacaagagcccattatttcttccacagaggaaaaggagagtataataagtaagtgtccagtttctcaagacatacaggcccctgcccaagagatccactttattttcatctcacccagaatattgaggtgatcagcaaggtggagtggttgggagagggtaaaagcaggaaagagagatggggactcaaacagcagcccatacttggaactgccatagatcctaccagttacttcatggactccatcaggaacccacctataagccacaggggatgcatccctcccatcttgccaacaaaaccccgaatgctccaaatgcctcacccactctttggctggctcccaagtgcgctcctgtgaaaagcgagtgagtatctctgcagatggcttgcaagcacatgttgacagctggctccactctgtagaactggaaaaaagctcacatacatgagaatttcaggacactaccctaaaaaaaaaaatgagacttcagcacctggcctggctttatgcaacctagagaaggtgatatgatttggctctgtgtccccacgtaaatctcatgtcaaattgtaatccccacgtgttggacaagggacttggtgggaggtgattgaatcatgcgggtggacttccctcttgctgttcttgtgatagagttgttatgagatccagttatttgaaagtatgtagcatgtcccccttcactctcttgcactcctgctccaccttggtaagacttgcttgctacccctttgccttctgccatgattgtaagtttcctgaggcctcccagctatgcttcctgtatggcctgcagaactgaactgtgagccaattaaacctcttttcttcataaattatccagtcttaggtagttctttatagcagtgtgagaatggactaatgcagaaggcatacaacctttagaatttgcccccttgagggaacaagatgtgtgaagcaggttcatccatagaaaatgtctgagagaacctcaaaatccctaacctgactaactgatgaaagtgtttctctcctaaggccagtcagtaaagaccagagggggtgactgtttctttaaatgcaaaggcagcagcacaataattcaagaaacatgaaaaatcaagaaaacatgacaccaccagaagaacacaatcattttccaataaccaactccccaaaaatggagatttacaaattggtttataatgaattcagaacaattatgttaaggaagctcagcaaactaaaaggaacaccaatagactactctgtgaagtcaggcaaacaattcatgaacaaaactagaaattcaaaaaagagaaaaattatcttaaaagaaaacccagaagttatggagctaaagaatacaatgcatgaaatgaaggagcgtatcaacagcaaagttgatcaagcataagaaaaaaaaaatctgtgaaactgaagactggctatttgaaattattcatcagaggattaaaaaaaaaagaatgaaaagaaataaagaaagcctacaggatgtataaaacaccatcaagagaactaatataaggattattggagtcataaaggagaagagagaaaagggtagaaaacttatttaagaaataatggctgaaaactctccaaatctaggaaaagatatgagcatccaggtatatgaagctcaaagatccccgtacaggatacattccaaaaagacttcaccaaaacacatgataatcaaactgtcaaaagcaaaatcaagacgatgaataaaccaccaatcactaagagggagacaggattcttatgttgtcattattatttacatataatttcagtaaatgttattggaaaatttataatgttttaaaaaaagaaatttgaaagcaccgaaagaaaagagactcatcacatacagggaacccttttaaggcattcaagagatttctcagtagaaaccttacaaaataggagagagtgggatgaactatacaagtgctgcaaggaaaaaaatgccaaccaacgctttacctggcaaatctgttcctcagaaatgaaggagagagaagaactttcctagacaaacaaaagctgaggcagttcatcaccactagacctgccttacaagacatactaaggggagttcttcaagctgaaatgatatggcaatagttagtaatatgaaatgataaacctcactggtaaaggaaagtacatagtcaaatttagaacactttgatactataatgatggtgtataaataattttactgtgctatgaaggttaaaagacaaaagtattaaaaaaaacccatagctgcaatagcttgtcaatgcatactacagtataaaaagatgtaaattagaacattaaaaacatagcatgcaagggtagggaagtaaaagtgtagttttcatatgtaatcaaatttaatttgttatcagcttaaaataaattgttatacctatgttttatgtaagtgtcatggtaactataaaggaaaaacctctagtagatacacaaaagaaaaagagaaaggaatcaaaacataacactacagaaaattatcaaattacaaaggaagacagcaagggaggaacaaagtaaaaagagcaagaaaaaatttaacataatgaaaacagtaagtccttacgtgtcaataattactttaaatgtaaatggattaaattatccaaacaaaaaaacagactggacaaatggattttagaaacaacaacaacaacaaacaccgcacacacacacacacacacacaaaccacccagccccaactatgtgctgcctacaagagatttacttccactttaaggacacatacaggctgaaattaaaagaacagaaaaagatattgcatgcagatagaaaccagaagagaggagaggcatctatacttacagcatacagaaaagattttaagttaaaaactatatcaaaaggctaagaaggtcaaaatggtgaagcagttaattgttcaagaagacataaaaattgtaaatatttatacacccaatattgaagcacctaaatatataaggcaaatattaatacatataaaaggagaaatatacagcaatacagtaatagtagtgaacttcagtgcctccctttcaaaaatggataatccagacataaaatcaataaggaaacatttaacttaaacttcactttagaccaaatggatctaacagacattatactgaacatttcatccaacagtggtagaattcacattcttctcaagcacacatggaacattctccaggatagattatatgttagctcacaaaataatattacaaaatttaataaagctgaaatatcaattattttgcaccacaatagtataacactagaaatcaataacaagatggaaactggaaatttacaaatatatagcattaacatattcctgaacaaccaatgggtcaaagaaaaaaatcaaaataattttgtgacagcaaagtagaaacacaacataccaaaacatacaggacacagcaaaagcagttctatgaggtaagcttatattgataaacacatttaaaaaaagattttaaataaacaacattacacctcaaggaactacaaggaagaaaaaaaaacaagccccatgttatcaaagggaaggaactaacaaagatcagacagaaataaatgaaacatagactagaaaaacaatagagactattaataaaacttagagttagttttttaaaaaataaaatcaacaaacctttagctagactaaaaaaagagaagactcaaataaaataaaaaatgaaagaggagacattacaactgataccacagacatacaaattaagagaaaactatatgccaacatattagttaacttgcaatgggtaaatccctagaaacatacaacctacaaaaactgaatcatgaagaaatggaagatctgaacagatcaataatgaataagggaattgaatcaatattcaaaaatctcacaaaaagaaaagctcaggatcagatggcttcactggtgaagactgccaaccatttaaaaaaattaataccactctttattaagctcttccaaaaaaattgaagaggagaaaacactttcaaattcattataagaggccagttttaccttgatatcaaagatttaaaaagaacactttgagaaaggaaaattacaggccaaaacccttgataaatatagatgcaaaaatgctcagcaaaatactagcaaacctaattcagcaacacattataatggcatacatcatgaccaagtgagattcatgcctcggatgcaggatagttcaatataatcaaatcaacaaatgttacactactttaacagaatgaaggataaaaatcatatgatcatctcgatggttgaactagtttacagtcccaccaacagtgtaaaaatgttcctatttctccacatcctctgcagcacctgttgtttcctaactttttacagatcaccattctaactggtgtgagatggtatcttattgtggttttgatttgcatttctctgatggccagtgatggtgagcatttttcaagtgtctgttggctgcataaatgtcttcttttgagacgtgtctgttcatatccttcacctactttttgatggggttgtttgtttttttcttgtaaatttgtttgagttctttgtagattctggatattagccctttgtcagatgagtagattgcaaaaattttctcccattctgtaggttgtctgttcactctgatggtagtttcttttgctgtgcagaagctctttagtttaattagaccccatttgtcaattttgtcttttgttgccattgcttttggtgttttagacatgaagacagtgtggtgattcctcaaggatctagaactagaaataccatttgacccagccatcctgttactgggtatatacccagaggattataaatcacgctgctataagccataaaaaatgatgagttcatgtcctttgtagggacatggatgaagctggaaaccatcattctcagcaaactatcacaaggacaaaaaaccaaacaccgcatgttctcactcataggtgggaattgaacaatgagaacacatggacccaggaaggggaacatcacacactggggatggttgtggggtggggggaggggagagggatagcattaggagatatacctaatgctaaatgacgagttaatgggtgcagcacaccaacacggcacatgtgcacatatgtaacaaacctgcacgttgtgcacatgtaccctaaaacttaaagtataattaaaaaaaataatgctgctataaagacacgtgcacacgtatgttcattgcggcactattcacaatagcaaagacttggaaccaacccaaatgtccatcaatgatagactggattaagaaaatgtggcacatatacaccatggaatactatgcagccataaaaaagatgagttcatgtcctttgtagggacatggatgaagctggaaaccatcattctcagcaaactatcacaaggacaaaaaaccaaacactgcatgttctcactcataggtgggaattgaacaatgagaacacttggacacaggaaggggaacatcacacactggggcctgtcgtggggtcagggtagggggagggatagcattaggagatatacctaatgtaaatgacgagttaatgggtgcagcacaccaacacagcacatgtgtgcacatgtaccctagaacttaaagtataataaaaaataaatcatatcatcatctcagtagatttagaaaagcatttaacaatattcaacatcctttcagaactaaaaactctcaataaatcaggtatagaaagaatgtgcctcaacactataaaagccacatatgacaaacctggaggtaatatactcaatggtgaaaagtaaaaagctttgactctaagatcagaaccaaaacaaggatgtccattctcaccacttatatttaacatagtagttgaaattctagctagagcaattaggcaagaaaaaaggcacccaagttggaaagaatgaagttaaattgtctctgtagatgacatgatcttatatatagaaaacactaaagactccaccaaaatgctgttttaattagagcttaaaaaaataattcactaaagttgcaggatacaaaatcagtatacaaaaatcagttgcatttctaaacaccaaaaacaagttatccaaaaaattaagaaaacaatcctatttgtgatatcatcaaaaaataaaatactaaagaataccaaagaaactgaaaataaatggaaataaatggaaagatagcccatgttcatggattagatgaattaatactgttaaaatgttcatactacccaaagcaacctacagattaagtgcaattcctactaaaattccaatgacatttttcacagaaatagaaaacacactcctaaagtttgcatggaaccgcaaaagactcaaatagatgaagcaattttgagcaagaacagtaaagctggagacatcacactacctaacttcaaaattttattaatcaaaacagcatgacataaaaacagacagaaaagaccaatggaacagaatagagagcccagaaataaactcacgtttatagagtcaactaatattcaacgaaggtgccaagagtacacaatggggaaagtatagtctctacaataaacagcactgggaagacaatatccaaatgcaaaagaatgaaattacacccttatcttataccatacacacaaatcaaatcaaaattgataaagacttaaatataagacctgaaaccatacaatctttaggcaaaaactcattgacattggtcttggcaatgatttttttgatatgacaccagaagcacaggcaacaaaagcaaacctaaacaagtgggaccctaacaaactaaaaagtttctgcacagcaaaggaaacaatcaatcatcagaattaaaaggcaatttatgaaatgggagaaaatgtttgcaaaccacatatctgataaagggttaatatccaaaaatatataaggaatgcataaaattcaatagaaataaacaaacaaataatccaattttaaaatgggtgaagaacctgaatagacattttttcaaagaagacttacagataggcaacaggcatatgaaaaaatgcttgacatcactaataatcagggaaatgcaaatcaaagctccagtgaaataccacctccaactattaggatggctattatcaaaaactcaaaagaaaacaagctgggggaatgtagagaaaagggaaaagaagatccttatacactgttggtgtgaatttaaactggaatagcccttatggaaaacagcatggaggttcctcaaaaaattaaaaatagaactactatatgatccagcaatttcactattgagtatatatccaaatgaattaaaatcactgtcttgaagaggtatttgcacactcatatttatttcagcattattcacaatagccaagacatggaatcaacctaagtgttcatcagtagatgattagataaagagaacgtggtatatagacacagtggaatctatttagtgttcaaaaagaaggaaatccaacttttaaaatcctttaaaaagttaaactcataaaaacagagaggagaatggcggtttccaggaactggagggtgggagaatggggagatgttggtcaaaaggtacaaactttcagttataaaatgaataagttctagagatctagtgtacaacagcattactatagttaataataatattttttatacttgaaatttgctaagagtaaatatcaatattctcaatacacacaaaacagaactatctgaaggcactgatatgttaattatcttcattatagtaatcatttcacaatgtataatgaatatcaaaacaatagtgtacatcttaaatatatacagttttgatttgttaatcatacatcaatgaagctagaaaaaatgttgtaatttttaaaacaatagtaatataaattaggggtgaaggaattgacctatgttggagaaaagtttttgtaaactatttaaattaattggtatccattcaagctagattatttttaattgttaatttaattgtaatactaaggcaaccactaaaaaagcctttaaaaaaatatagcacttgaggctgggcatggtggccctcaattatatatataatatatataaaatatatattatatatatagtagatgaacaacaatgggatttaaatggtacactagaaaatatctgtttaacaaaaagaaagcaatagtggagaaatataagaacaaaaccatgtaagatttatagaaaatgaatagcaaattggttgacctaaaccctatcttataattatattaaagataaatgaaataaatactacatcaaaaggcagagattatcagaatagagaagaaaaatccaaaccataattcaaccttatgttatctgtatttagaatatttagagtcaagacacaaatagatagagttccatttgggcgtgaaaatatttaccatgcaaaatgtaattaaaatagagctagagcagcaatactaatatctgacaaaatatactttaacaaaaattgttgctaaagacaaaaaagaacattttataataagacacaacaattataaacatatacaccaaacaatagagcccaaaatatacaaagcaaaaactgctagaattgaagaaagatagaaaaatcaatgattacagttggaggtgtcaataccaaactttcagtaatacacagaacaacaagtcaaaagcaaaaaggaaataaaaaactacacattatcatacaacaccttaatacgatatccaacaatagcagaatacacattattctgaagtgcacatggaatattcttcaggatgacacatattaggctgtaaaacatatcttaataatttaaaagaattgaaataatacagcacatattctctgactgcaaaatattaaaccaattacagaagaaaatgtgggaaattcacaatgatatggagatttaaaaatacttcaaagtaaccaatgaatcaaagaacaaatcacaagagaaattagaaaatattttagatgaataaaactgaagaaacaacataccaaaattttgtggttgtagctaaagcagtgcttcaagggaaatgtataacagtatatccctaaattttaaaaaaatctcaatccaataacctaatttttcaccttaagaaatgagagtgaaagagcagacttaacccaaagtaaacagaaggaagaaataataaagattagcatggagataagtagtgaaaataaaaacaatagagaaaattaataaacttgaaagttggttcttctgatatatcagtaaaattgacgtattattagtttgaccgagaaagaacaaaagagagaagattcaagttactagacataaatgaaagtatagatatcaccttacagaaattaaaagaattctaacagaatattgtgaaaaagtgaatgtcaaaaaattaaattatatgagatacacaaatccctctaaaggcacaaactaccacagctggtgtaaaaacatgaatacaccatttacagttaaaaagactgaatagataaaaattttaagaagaaattgagtaagtaatttaattttcaaactataatcccaagaccagatgttttcattggtgaattctaccaaactttaaaaggattaatatctatttttcatacactctttccagaaaatagaaaaggagggaacactctataactcactgtatgaggtccgtattacccttatatcaagaccaaacatcataagaaaagaagactaaagacttatggtttctgctctgatatgtttggaagtcatcactactattgtcacaaggaaaaatctgaacaaactaaagtcaacgatttcttaaactaaccatagaattgaggtaacgggcgaaaactggagatgtaggcaaatacaaaaaaaatcacagtttatcaggagcagaaactgctgaaaccagcaactcgtatgaacatgttaagtggtaattgacaaatttctggagattgaatgtggactggattgagagttaggaactcctaagtgcccagtttttgatgaccccacacacttttgtaaactagacttccaagagccccagcaagtttcttacagtgaagactgcagaaaaatcccctgatgcttcagataggaggaagggaaaagcaactattttgaaataagcccaggggacaagtagttatttctaaacactctcagagcattttctttcacacggcagggggctccctgcaagggaagctactttgcctgagccttgtctgatgtaggagaaaaggaattggatggctcaagctccatctagcctttctgatttatataagggaagcaaaaaataggttaagaaactcttctgaaagtcacaactcagatttattttatatttatttatttatttatattttttgagacggagtctcgctctgttgcccaggctggagtgcagtggcgcgatctcggctcactgcaagctccacctcccgggttcacgccattcttctgcctcagcctcctgagtagctgggactacaggcgcccagctaattttttgtatttttagtagagacggggtttcaccacgttagccaggatggtctcgatctcctgacctcgtgatccacctgcctctgcctcccaaagtgctgggattacaggcgtgagccaccgcacctggcctcacaactcagatttaaccataagattatagaacacttctcctcccaaaacagagatatagatcaatggaacagaacagagccctcagaaataacgccgcatatctacaactatctgatctttgacaaacccgagaaaaacaagcaatggggaaaggattccctatttaataaatggtgctgggaaaactggctagccatatgtaaaaagctgaaactggatcccttccttacaccttacacaaaaattaattcaagatggattaaagacttaaacgttaggcctaaaaccataaaaaccctagaagaaaacctaggcattaccattcaggacataggcatgggcaaggacttcatgtctaaaacaccaaaagcaatggcaacaaaagacaaaattgacaaacgggatctcattaaactaaagagcttctgcacagcaaaagaaactaccatcagagtgaacaggcaacctacaaaattttcacaacctactcatctgacaaagggctaatatccagaatctacaatgaactcagacaaatttacaagaaaaaaacaaacaacctcatcaaaaagtgggcaaaggatataagcagacacttctcaaaagaagacatttatgcagccaacagacacatgaaaaaatgctcatcatcactggccgtcagagaaatgcaaatcaaaaccacaatgagataccatctcacaccagttagaatggcgatcattaaaaagtcaggaaacaacaggtgctggagaggatgtggagaaataggaacacttttacactgttggtgggactgtaaactagttcaaccattgtggaagtcagtgtggcgattcctcagggatctagaactagaaataccatttgacccagccatcccattgctatatatatatatatatacccaaaggactataaatcatgctgctataaagacatatgcacaggtatgtttattgcagcactattcacaatagcaaagacttggaaccaacccaaatgtccaacaatgatagactggattaagaaaatgtggcacatatacaccatggaatactatgcagccataaaaaagatgagttcatgtcttttgtagggacatggatgaaattggaaatcatcattctcagtaaactatcgcaaggacaaaaaaccaaacactgcatgttctcactcatagatgggaattgaacaatgagaacacatggacacaggaaggggaacatcacactctggggactgttgtggggtcgggggaggggggagggatagcattaggagatatacctaatggtaaatgacgagttaatgggtgtagcacaccagcatggcacatgtatacatatgtaacaaacctgcacattgtgcacatgtaccctaaaacttaaagtataataataataaaaaaaggctacctaaaaaaaaaaaaaagaacacttctcctcccaacaccatatcaccacatcaaccaggactccagtgtaatagcagtgaattctaactgaaagaggtgaaagacactgattgtatttaagaaagatcttctaaggaaatccaaaaatagtaggggagatcaaaacaaagatactagaggaaattgaatatgtgacacctatagctacaaaaaaattaaacataacatagccctaaccatataaacataaaacctcacacaaagacctattatctgagattctgttgcctgatacattgcgttttatttcaataaaaaaattagagggtatgttaaaaagcaggaaaagttagtctaaagagacaaattgagcctcagaagtaggctcagatatggcagagatttggcaattatacctagagtttaatataaatgattaatataataagtgttctaacagaaaaaggcaacatgcaagaacggatgggtaatgtgatcagcaagaaggaaactctaagaaagaagtcaaaaggaaatgctaggaataaaaacctacaagaaataaagaatgcctgtgatgggttcctcagtagactggacaaggtcaaagaatcagtggatttgaaaatatgtcaacaaaaactgccccactgaaatacaaaagaaaaatagaattttaaaaacgtaacacaatctccaaaacagtgggacaattacaaaagatgtaatgtgcctaatgcaaatgacagtaggagtataaagggagaaaggaatagaaaatctgaagtaataatggcagagagttttccaaaattaatgctaaaccacagatacagcaagcccagagaacaacaaggaggaaatttagtaaagcgtctgcaaccaagtatgtcatattcagactgacaaaaccaaaggtgaagagaaaatattgaaagaagacaaagaggaaaataaatatcaagaaaatacatacgaaatacatcatacatacataagaaatacatcagaccatacaagcaagaagagaatggagtgaaatgtttaaaatgttgaaaaaaaaactatcaatttgcaattctgtatccagtgagattatctttcaaaagtgaagagggaaatggcagagaagtcatctccaagacctatggtttcccttcacagaaacactgaaaaatatgaacaaaagtggtcagaattaactttctaagaattctataaaatggtaaaatgtttacaccagtaaagcaaatgctgaattgagaaggcaacttaaaaaggtgaagaaaacttcgtattatttttatgtgtccttgccccacgtccttccctaccttagtcttgaagatggcagcccacatttctactgtggggctctggtttctgtttcctggttcaagagggagaataacagaccttacttttagtcattattatttccttctttctgatttccttgggtttattttgctcttctttctactttattgaaatgagaactaagattatgatttgagacatttttctaatgtaagcatttagtgctataaatttccatctcaacactgctttagtcacatcccacaaatttttatatgttgtaatttcactttcatttagttctatttttaaattttttctttttatacttcctctgactcacagattacttagaattgtgttgttcagttttcaaggatattgtagattttcctgtttctctgttgtctaatagttctgttccattttgtacagatagctcacgctgtatgatttcaattttttaaaaaattgtgctttgttttatggcccagatatggtctgtgctgtgaatattccatgttattataaagtatgcctgttatattattatatatatataatatatataattataaagcatgcccgttttgtattgttagcagagtattctagaaatgtcaatgagatcttgttggttgatggtgcttttcagttctatatctttgctaattttttttttttttgcttagtagctatatgagattctgagagaggaaattgaagtctccaaccataattgtggatttgtctatttctcctatcagttctatcagtttgtgcatcacatatttgaggctctgttgtttggtgcatacacaagtggaatcattgtgccctcttggtggcttattttatgattatatagtgcctatctttgtggtatttttatttgctcttaaatctactttgtgttatattcatatacccattcttttttaaaaaaaattgtttgcgtgatacatcttttccattcttttaatctcagcctatctgtgccattgaatttgaagtgagttttcatatagagaacatattattgaatcatcattttaaaaattccttttgccaatcttttttatactgaggtaaaattgacataaaatttatcattttaaagtgtacaatacagtggcatttggtaatacacatgttatgcaacgttaactctacctggctcctaaatgttttcatcatccccaaaaggaaacttcatactcattaagcagttaattcccattccttctctcggccactggcatccgcaaacctacttttctgtctctatgaatttacctattatggatattttgtataaattgaattatacaataagtgacctttatgtttggcttcgttcgcttcgcatactatttttcgatattcaaccatgttgtagtatgtatcagttttatttgaataactcaattctttttgttgtatagctaaaagttgattcctaggtcataatgataattctatgtttagtttattgaatagctgccaaagtttttccacagtggctctgtcattttaaaatcccactagcaatggatgagagttccaatatctccacatccttaccaatattgttattttatatttttataattataattttcctagtgaatacgcaatggtatctcattgtgtttttggtttgcctttccctaatgactaatgatgttgagcatcttacaatgtacttgttaactatttgtgttctttagagaaatgtctattcaagtgccttgtccattttaaaaatcgagttgtcttgttgacttatgagttctttaatacagtaaacgcttatcagatatgatttataagtattttaacccattctgaaggtcaccttttcacttttgtggtagaccattatgcacaaaggttttaattttgataaatccaatttatccgcttttgttgttgtttttgttgttcgtgcttttgcaaaacctagtgtcatgaggttttctcattatctttggagaattttatagtttgggtctatacatgtagattattgatctaatttccattaatttgtgtgtatgctacgaggtaggggtccaaattcaatttttgcattgaattgaaaattcatattttcagtttcaaatttcaactgcatattcagttgttgcagcacaatttgttgaagagatcattctttaccacagggaatgatctgggacccttgtcaaaaatcaattgatcatagatgtatgggattatttcagactttagatcttgttccatgaatatgcctatttttatgccagcactgcagtattttcattactgtagctttatcataaattttgaaatcaggaagtatgtatcctccaagtgtgattttcatttgctagagtgttttgactatttggggtctttgcaattccatatgaatttcagaattggcttttcatttaaaaaaatggtagttgagattttcataggaattatattgaatctacagatcactttgggtagtattgccatcttaacaatattgtattccaatccataaacacggatgtattgccattcatatctttttttcttttctttcggcaacatttagtatatgacacttgtaactccttggttaaatttatacctaagcattttatcctttctgatgctgtttaaatggaattattttattaatttctcttttagatggcttgttgcaggtgtatagaaatagaactgattattgtgcttttattgaattatctgaaactttgctgcatttattaactctagtaggttttttttttctttaaagttgtctatatatcttgctctgtgaatagataattttacttcttgatctccaatatggatgcctttctttctttttcttacctaattgctgtagctagaattttcagtataatgtatgatagaagttgttcacaactatccttgtcttctttctgatcctaggggtatagctttcagtctttcaccattaagcacaatgttagctgtggggttttcttagatgccatttaatatattaaggaagtgctctgctatttctaattggttgagtatttttattataaaatggtgttagattttatgactttatgtactgcataattgagattatcatgtggtcttttcattcgattaatgtggtatattttatgattttcatatgttgatccacctttatattcttggggcaaatcccactgtgtacacgggttttttagggtctcctaattttcttcattctttttcttttctctccctgagactgaataacgttaactgacctatcttcaagtttactattttttcttctgctgttcaaatctgcttgaacctatagagtgaatttttcattttacttattgaacttttcagctccaaaatttctctttggctactttgtataatatctatctctctatagatattctctatttggaaagacatttttctcctggttttctttacttatttgtattttttaaagtctttaagcatatttgagacagttgatttaagtatttgtgtactaagtccattgcctaagcttttgcatagagattttatattaatttcttttttttctgtgaacaggtcttttttttttttttttttttttttttttttttagaaaactggacattttgagtagtataaacgtggctattttagaaataattttcctccctccttaggttttgtttccacttgttgcatgttgctgttgtttgctcattagtgacttttctaaagtattttgaaaagtctgtgttcttgatcatgtgggtccattgaattctgtattctgttaatttcatagtcagctagtgttctgaaagttcccttaagtgcatagagccaataaaagaaaaagaaaagaaacacagaaaaagaaaagaggaaaaaaggagggaaagagatttaaaaaaataatgtcggttgggcatggtggctcatgcctgtaatcccagcacttttgggaggccgaggcaggcaaatcacttaaggttgaccatcctggccaacacggagaaaccttgtctctactgaaaatataaaattagccgggcatggtggcacatgcctgtaatcccagctactcgggaggctgagaggtaggagaatcacttgaactcgggaggtggaggttgcagtgaactgagatcacaccactgcactccagcctgggcaacaagaatgaaactccatctcaaaaaaaaataataataataatgcctttgccgatttgctctgtgtttgggccctcattcaatgcctagtcaggccatttacaactctctcttaactttcactacctgcttgtgtactgactgaaggccacctataggtgaaagcttaacatcatctcagatctttttggaccatgaatcctaccctgggtatgcacatgatcttcttaatttcccagtagatgcaagagttttagtgttttaaaagtccttattccctcatctatcttcttttctgacctttttcagtctgcttattgttcatctgaactgacatcctttgccccaagcggctgtggcaaaaacttttacctttaaatgctttcaccacaagccacttgggaagctgccccagacctgggactgctctgaccctgatgaaacaaagacaagaccttgtacagccaggcagccataagacaagtccatacccaaaccacagttctttcagaataaggtctatattggattctctggccctagtaaccagcatgagtctgggcttgccatcttcatggccacttgtcttagtttgctagggctgccataacaaaatatgagagactgagtggcttaaacaagagaaatttattttttcacaattctgaagaccagaagtccattactctgaaatctctctccttggcttgcagatactgccttcttgccgtgtcctcacacagccttttctctgtgtacacatccctggtgttttttaagcctgtccaaattttctgttcttctgaggacaccaatcagattggattagcgaccacccatatgatcattttaccttaagtacctcttcaaaggtatcagagtagtatatttgctacagttgatgaacctgcatacaacatcatcacttgaggtctgcagtttacattagagttcatcgttagtgttatatattctagggtttgttttgttttgagacagaacagagtcttgctgtgttacccaggctggagcgcagtggtacaatctcattgctacctctgcttcccagattcaagcaattctcatacctcagcctcctaagtagctggaactacaggcgcacaccaccatgcccagctaattttcgtatttttagtggagatggggtttcaccatgttggccaggcaggtcttgaactccttgcctcaagtgatctgcccgcctcagcctcccccagtgttgggataacaggcatgagtcaccatgcctggccttattctatgggttttgaaatgtataatgacatgtatccatcgttgtagtattagatagaatagcttcattaccctaaaagtcttctttgcactgggttgagttttaaaagctctttgattattttatgacagttccttattagatatatcttttgcaagtatttttatcagtctgtggttatcttgtcttttacagagcagtaatttttaattttaataaaatccaatttgtcaattacttatctcatatgactctggtgttacatctaaaatgttaccaccatactcaaggtcacctaggttttctctaggaattttatggttttgcattttacatttggtgtatgacccatttgaagttaatttttgtgaggttgtaaggtctgtgcctagattcattttttttttttttggcatgttacttcagtattcataagaaacattgttctgtaatcttctttcttatagtatcttagtcttgctttagtttttgggcaatgctggcctcactgaataaattcaaagtgttccctcctcttcaattatttggaaaagtttgagaaagactattgttaactgttttctaaatttttggtggaatttaccagtgaaccaactggtcctaggcttttctccagtaggtggttttgattatgctttcaatctctttacaagttacacatctatacagacttttataattcagtcttggtaggttgtgcgtatttaggaatctgaccacttcatctaagttatccaattagttggcatgcaattattcgtagttctctgaataatcatttttatttccacaaaattggtaatatcccagtttccattttttatttcattgaatcttcttttttcttagctaatctagctaaatgtttgccaattttgttgatcttttggaagaaccaacttttgatttattaattttctctactctttttctgttctttatattatttatttccacactaatctttactattttcttccttctgttggcctttaattttttttttttaatttttaaggtgtaaatttaggttgagaaattttttaaatgaaagcatttagagctataaattttccttctggtgttcctttcactacctgccataaattttgatatgttgagtttttgtttgtcttggagtattttctaatttgtcttctaatttcttccttgacctattggttatttaaatgtatttaatttttgcatattgtggatttcccagttttccttctgttattgatttctagttttattccattgtgatcacagaagatattttgtataatcgcagtctattaacatttattaagtcttgtggcctaacagaggatctatgttggagaatgttccaagtgcaattgagaatactattctggtgctattaggtgaagtattctctatatgtctgttaagtccaattcatctatagtgttgacgtttcctgttccttactgattttctgacttattattctatccattattaaaagtggagtggtaaagtctctattattgtagaactctctgtttttcaagtctatcaatatctgtttcatatattttggagctctgtttgctgcatatgtgtttacaattgttatatcttcttggcaaattgaccagtttcatcaacataaaatataattcttattgtcttctaacagtttatttttctttttacataaagcctattttatctgatcttagattccctcacactccaccccagcacgcttttggttacatttacataatatatcttttccatcctttcattttcaacctgtttgtgtctttagatctaaagtgaatgtcttacagacagcataagctatgtcattaaaaaaatccattctgcttatctctgccttttgactggggagtttaatccatttgcatttaaagtaatcactgatcattaaatactttcagtattttgttgttttatgtatgtcttataactcttttgctcttcatttccttcaatattgcctttgtgtttagcttatctttttgtgtcacactttgatccccttctcatttcttttttatattttctttgtggttaccaggaggacaatgtatcaacttttaaagttattacaattttatttttttaaatctctcccattcggggattttaggaaggttaaataataatgtaaatgagatacctagaacaatataagcattcaggaattattaactcaattccaatccttcctccacctccacctctttctctgtgagattatagaaaagatgacaaaaaggatgttttctgagccctttaattgttgagaatgatctttgagaaaaagaaaaaaaatgaaagcactaggaatgtacaacagcctggaagtataattaagtgtaaattaaatagataaaagttataagcagaggaaagtatagtagaactcagtatttaaaagagaatcaatgtgaaaattatataaatttatgtaaaataaaactaccagacaaatctgatatccttaggatttttctttctttcatgtgatttctaattgctacatatgacactaaaccattgatctgagctgtaagagaaactggaaattgttctgttatcttttgtaagatttctagaacattttgccctcagacttaaatgccaacgtatttctcacttattgtttactgcttttggatttacatatgatttgattctttcttatctcttatccttacaatgtaattcaaactgatgccaatttaagttcaattgcgtacaaaaactctactcctatgcagctccgccccatctaatttacattattgatgtcgcaaattccatctttgtacatagtttacatattaacatggatttatacatttttatgtatttggtttttaaatcctgtagaaaataaaaagtcaacacaccaatattaaaataatactggtttttatatttgtccatgtgcttacctttatcagtgttctttacatctttatacgggtttgagttactgtcttgtgtcctttagttccaacctaaagaactccctttagcatttttatagggcaggtctagtggtaatgaactctctgagtttttatttagggatgtcttaatttctagctcctgtttgaagtaaatttttctggatatacaattctctgttgattgatttttgttcattttcccttcagctcttttaaatacattatcccactttcttctgccttccaaggtttctattaacaaaattcagcttataatcttattaaagatctcatgtacatgagtggcttctctcttgctgttttcaagattctgtgactttggtttctgatagtttaaatataatgtgtcttattgtgggtctctttggatttatcctagagtttcttggtcttcttacgttggtatatccatgtatttcaagacatttgagtagttttcagccattatttcttcaaacaatctctcctctttggggacttccattttacctatattggttcttttgatggtgtggcaccagtcccctagactttgttcacttttttccagtcttttatttctgcttctcagactcaacagcttcaagtgttctgtattcaagtctgctgactctttcttcttccagctcaaatctgctgttggatccccccttgtaaaatttttaattccattttagtgtttttcaagttaagtatttttattaggttcctttttataatttctttttgttgatattctcattttattacacataatttgtctgatttccattagtttttttctttgttttcctttagctctttgaaaatatttaagacattttaaaagtctttatccaagttcaatttctatggttctgtagagatattttctgccagtttatgttcttcttttccatgggccatgttttcctgtttctttgtatactttctaatttttggttgaaaactgagcatttgaaaatagagccaactttcccagtttctgcagagagtctttatgccacagtattcgttcactgattaactgggtatatctaagcttagggagcagctgagtcaaaagtttaaggtcttctcaggtcttttctgagtatacatgtttcctatgcctgtgtgaaatgttctcaatttccctatataaacagctacttcttcttttttttttttttttgagatggagtctcgctctgtcacccaggctggagtgcagtcatctcagctcactgcatcctccacctcccaggttcaaacaattctcctatacaggtgtgtgccaccacgtctggccaatttttgtatttttagtagagacagggtttcgccgtgttggccaggctggtctcaatctcctgacctcaggaggattacaggcttgagccacagtgtccagcctaaacagctacttttgaatgctttaatttcctgaatagtctcaacccagtttttccttgaggtcttaggtggtccattgtatgtctccacccatagttgcttgccccaggcatctgtgggtctgtggtaccactgcagctttcaccacctgtagctgccacctttccctatctgagatccaggttaggtgagagagatcattccttcacgcagtcccatgacaggttggaacatttcaaataaggtctgttctgctcctctggttgaagggagaaaattgggaaccggtttcccaccttctacaaaccaagatctcatgttgccacgggagtggcagggcaagtgcaagtgaaaatgccatacaattttctaccattttgaacgcgggtttttcttcaatggtcatttgcttggttgctgtaggcctttcactgttttccagagctcccataagattactttagccagttttttgttctttcctgatgcttccctggcagagtaagggttggaacttccaccattttgctgattcataactctgtagtcagttttaaatatattgatacttgagtttgttttatggcccagaatatggtcttggtaaatgtttcacatatactcagaaagaatgtgtattctgatgttgttacatgggctgttctataaatgttacttaaggtggttaataatgttgctcaagtcttctatattcttgctgattttcttttatttatttattttattttttttattatacttttaagttctagggtacatgtgcacaacatgcaggtttgttatatatgtatacatgtgccatgttggtgtgctgcatccattaactcatcatttacattaggtatatctcctaatgctgtccctccctgctccccccaccccatgacaggccccagtgtgtgatgttcccctttcctgtgtccaagcgttctcattgttcaattcccacctatgagtgagaacttgcggtgtttgtttttttgtccttgtgatagtttgctgagaatgatggtttccagcttcatccatgtccctacaaaggacgtgaactcatccttttttatgactgcatagtattccatggtgtatatgtgctacattttcttaatccagtctatcattgatggacatttgggttggttccaagtctttgctattgtgaacagtgctgcaatgaacatacgtgtgcatgtgtctttatagcagcatgatttataatcctttgagtatatactcagtaatgggatgggtgggtcaaatggtatttctagttctagatcttgaggaatcaccacactgtcttccacaatggttgaactagtttacagtcccaccaacagtgtaaaagtgttcctatttcttcacatcctctccagcacctgttgtttcctgactttttaatgatcgccattctaactggtgtgagatgggatctcattgtggttttgatctgcatttttctgatggccagtgatgatgagcattttttcatgtgtctttggctgcataaatgtcttcttttgagaagtgtctgttcatatcctttgcccactttttgatggggttgttttgttcttgtatatttgtttgagttctttgtagattctggatattagccctttgtcagatgaggagattgcaaaaattttctcccattctgtaggttggctgttcactctgatggtagtttcttttgctgtgcagaagctctttagtttaatgagaccccatttgtcaattttggcttttgttgccattgcttttggtgttttagacatgaagtcctcgcccatgcctatgtcctgaatggtattgcctaggttttcttctagggttttttatggttttaggtctaacatttaagtctttaatccatcttgaattaatttttgtataaggtgtaaggaagggatccagtttcagctttttacatatggctagccagttttcccagcaccatttattaaatagggaatcctttccccatttcttgtttttgtcaggtttgtcaaagatcagatggttgtagatgtgtggtattatttctgagggctctgttctgttccattggtttatatctgttttggtaccagtaccatgctgttttggttactgtagcctcgtagtatagtttgaagtcaggtagtatgatgcctccagatttgtccttttggcttaggattgtcttggcaatacaggctcttttttggttccatatgaattttaaagtagttttttccaattctgtgaaggaagtcattggtaacttaatggggatggcattgaatctataaattaccttgggcagtatggccattttcacgatactgattcttcctatccatgagcacggaatgttcttccatttgtttgtgtcctcttctatttcgttgagcagtggtttgtatttctgcttgaagaggtccttcacgtcccttgtaagttggattcctaggtattttgttctctttgacgcaactgtgaatgggagttcactcatgatttggctctctgttagtctgttactggtgtataagaatgcttgtgatttttgcacattgattttgtatcctgagactttgctgaagttgcttatcagcttaaggagattttgggctgagacgatggggttttctaaatatacaatcatgtcatctgcaaacagggacaatttgacttcctcttttcctaattgaataccctttatttctttctcctgcctgattgccctggccagaacttccaacactatgttgaataggagcggtgagagagggcattcctgtcttgtgccagttttcaaagggaatgcttccagtttttgcccattcagtatgacattggctgtgggtttgtcataaatagctcttattattttgagatatgtcccatcaatacctaatttattgagagtttttagcatgaagggctgctgaattttgtcgaaggccttttctgcatctattgagataaacatatggtttttgtctttggttctgtttatatgatggattatgtttattgatttgtgtatgttgaaccagccttgcaacccagggatgaagcccacttgatcatggtggataagctttttgatgtgctgctggattcagtttgccagtattttactgaggatttttgcatcaatgttcatcagggaaattggtctaaaattctctttttttgttgtgtctctgccaggctttggtatcaggatgatgctggcctcataaaatgagttagggaggattctctctttttcctatttactgtacatttattccaccagtgactaaaacaggtgtatcaataaaatctgttccctcaggttttgcttcaggtattttgagggtctgttatcaggtgcataaacaagattgttatgtcctattcttaaattaatctccttataattatgaagttaattttttttttcttgagatgcagttttgctctgtcgcccaggctggagtgcagtggcacaatctcggctcagtgcaacctctgcctcctgggttcaagcatttctttgcctcagcctcccgagtagctggggttacaggtacctgccaccacgcccggctaatttttttgtatttttagtagagatggggtttcaccatcttggccaagctggtcttgaactcctgaactcttgatccacccaccttggcctcccaaggtgctgggattacaggtgtgagccactgcgcctggacctggcccgaagtaaacttctttacccttgctaatgatctttgctctgaagcatgctttgctggtattaatatagtcattccttctttctttgattcatgtttgcagggtatatctgtttccattcttttacttttaacctattgtctttatatttaaagtgcatttcttgtaagtataattggtttcttaaaatccaattatctgccttttaaatgtcatttttatatgatttgcataaatatgattattattacagctaaattgaaatctgtcatcttgctatttggtttctatttatcccatttttttcccctctttttttgctttccttgagattgaacattgtattagttttctagggttgctgtaagaaagtgacataaagtggatggcttaaaacaacagaaatttattgtttcagtttggaggctagtcatctgaaaccaaggtgtcatcagggccgtattctctctgaaacctgtagggaagaattctttcctgcctgttctagcttctagcattttccagcaattcttggcattcctttgcttgtagatgtatccctccaatctctgcctctatcataacatagccatcttctccctttatctgtctattcttctcatcttataagaacattaattactgaattggggcccagcatagattaggcctaatctcatcttgaataggttacatctgccaaagattcttcttccaaataagatcacttttacagcttttacaggtactgagagttagaacctcaatatatcctttggtggggaccgacctcttacccataaaaagtattttatatgattccattttatctcctttttaggttattaactacaatttttttttcttttttttgagatggagttttgctcttgtagcccaggctggagcgcgattttggctcactgaaacctctgcctcccgggttcaagtgattgtcctgcctcagcctcctgagtagctgggattacaggcatgtgccaccgtgcctggccaatttttgtatttttagtagaaacagggtttcaccatgttggctagggtgggtctcaaattcctgaccttaggtgatccacccacctcggcctcccaaagtgctgggattacaggcgagagccaccacgcctggctttataatttttttttaattcagtggatgttttagggtttatagtatacatctttatcacagtctagctccaagtgatatatccctttatgtatagtacatgacccttacagtagtgcatttccatttttcctctctggcatttaggctattgcacacacatacactcaattcatccctttgtgtagatccgtatttccagctgctatcttttgcttctgtctgaaatatatgtatgatttcttttatgactattttgagaaatttggttatgtgcatttgtctatttttcttatgtacttttagctcatcaatcttaagtctgtgagtttatagtttttaaaaacaaatttgaaattatttggctattatttcctcaaatatttttttctgccgcccctgctttccctttccttagggatctctgatttccacctatattactctgattgaagttgttccacgtctcttttgaaaatctctgaaaaatcttttatcatttggataatctgtatttgtacatcttcaagtacattaatattttcttttgcaatgtttaatctgctgttaatcccatgtagtggattcttcatctcaggtattgctgttttaatctctagaaattccattgaagtcttacttgatgaaaaaggcagataaaaacaaatgttggcaaagatatggagaaatcagaatctgcacacactgatgatgggaatgtaaaatggtcaaggcaatttggaaagcagtctggcagtttctcaaaaggctaaacgtagttcccatatgacagcaatcattcatctaggtatatactccagagaaataaaaacatatccacaccagaacttgaacattaattttcatagcaacattattcctaggggtttaaaattttttacattatttttatttaaaatagagacagggtctcactacgttgcataggctggtctggaactcctggaattaagcattcctcctgcctttgtcctgttttctccactggaaaagaataaaactttgtacacttggactaacaactcccgattccctcctttaccaacatgccccacagcctctagtaacttactctctactttcatgaattcaacttttttaagattccacatataagtgagatcatacaatatttgtctttctgtgcctggcttatttctcttagcataatgtcctccagattcatacatgttgtcttaaatgacaggatttaccttcctttaaaggctgactagtacttcattgtgtatatgtatcacattttctttatccacttatctgttgatgggcacttaaattgtttcaatgtcttggctactgtaagtaatgcttcaataaacatgggaatgaagatatcccttcaacatattgatttctgttcttttggataatactcagaagtgagattactggatcatatggttgttctatttttttcagaaacctccatactgtttttcatagcggctgtactaatttacattcccactaacaatgcatgagttcacttttctggacatcctccccaacacttgttatctttcatctttttcataaaagccattatataataggtgtgaagtgacatctcactgtggttttgatttgcattactctaataattagtgtgagcattttttttttttcatgtaccgaatgtcttttgagaaaggtctcttcattcctttgcccattttaaaatcaggtggttttcttgctcttgagttgtttgagttccttatgtattttagatatttacccatttccagatatatcatttatattttttcctattctttgagttccctcttcactgtgttgtttccattgctgtgcaggtcttttattttgatgccaccccatttgtctatttttgccatgcttttgcagtcatatccaaaaaaatcattcccaagaccaatgctgtggagatttccccctatgttttcttcagtaggtgtacagttttaggtcttatatgttaagttttaaatctatttttttatatggtgtaaataagggtctaatttaattcttttgcatgtggatatccagttttcccaacaccatttattgaagaccctgtccttttatacttttcagtatgcagatcttttacctccttaaatttacacctcagtatttaatatttgttgctattatgagattttcataatttccttttcagatagctcattaatagtagatggaaacactactgatttctgtaagatgattttgtattacggaactttactgagtttgtgtatcagttctaccaggttttagttttgttctggtggagacattacagttttttgtatatggttatgtcatcagtaattacagatgatttaacctattcctttcctattaggatgccttttttttctttctcttgtccaactgctctggttaggacttctagtactatgtcaaaaagtgatgagggtcgtacatggcctccatacctaatctgttgagagtttttaccatgaaaccaggttgaattttgtcaaatgctttttctgcatccattgagatgatcatatgatttgatttacaccctccattttgttatgtggtatatcacactttttgatgtgcatatgttgaaccacccttgcatcctaaggataaatcccacttcatcatggtgaatcattctttgtattcgtgaatccagtttgctaatatattgttgaggatttttgcatccatgttcatcagggatattactttgtaagtttctgtccttaaagtgtctttctctggctttaataacagtgtaacactacccttgtaaaatgaatttagaagtattccctctgcttcattgttttggaaaagtttgagaatttttattagttctttaaatgtctggtaaaattcagtagtgaagctgcctaatcctgggctttcctttggtgggatactttttattactggctcaatctcttttcttgttattggcttattcagatttgtttcttcatgattcactctttgtaggttgtatatgtctaggaatttattcatttctttaggtcatccaatttgatggtgcataacttcatagtagtttcttataatcctttgtattttggtgatatcagtagtaaatgtctcctctttcatttctgatcttatttgagtactctttttttctcctagtctaggtaagaatttgttgattttatctttcaaaaaaaaaaaaaaccaactcttagcaactcttagttttgttcatttttttccagtctttatttcaactgtgatctttgttacttacttctttatgctaactttcgggcttagtctgttcttttcctagttcctttaggtgaaaagtgagattgtgatccttcttctttattggcgtaggtttgtatcgctataaatttccattaggactgattttgctgcatcacataagttttgtttccattttcatttgtctcaaggtaattttttatttactttttgacttcttctgtgaactattagttgtttgggagcatattgtttaatttccacatattgctgtattttccaccagaattgattcttgttcttgatttctagtttcacgccattgtaatcagaaaagggatttgatatgatttctgtccacttaaacttaagattagttttgtggactaacatatatcctggagaatgttccatgggcatttgagaacaaaatgtattttgctgcttctggatggaatgtttcatatatgcctgttaagtccgtttggtctaaagtgtaattgaaatccattgtttctttattgattttctgtctaggtgatcaatctgcccatggtgaaaagtagagtattgaggtcccgtattatagtattgcagcctatctccctcttcacatcatttaaaaattgctttatgtatttaggtgggtcaatgttgggtgcatatacttttacaattgttatgtcttcttggtgaattaatccctttatcattatataacaaacttctttctttttatagtattgacttaaagtctattttgtctgatagaagtatagctacccctgctctcaatttccatttatatagaatatctttttccatcccttcactttcagtctatgtgtatgtttagtagaaaagtgaatctcttgccgggcgcagtggctcactcctgtaatcccagcactttgggaggccaaggcggcgggatcaccagaggttgggagttagagaccagcctgaccaacatggagaaaacctgtctctattaaaaatacaaaattagccaggcgtggttgtgggcccttgtaatcccagctactcgagaggctgaggcaggagaattgcttaaacccgggaggtggaggttgcggtgagctgagatcatgccattgcactccagccttgacaatagcaaaactccgtctcaaaaaaagaaaaagaaaaagaaaaaaaagtgagtctcttataggcagtatgtggttgtgacttaaaaaaaaaaaaaaatccattctgtcattctatgtctttttgttggagaatctaatccgtttacattcaaggtaactattggtaagaaactgctagtgtcattttgtaatttgttttctgattgttttgtaggtcccttgtttcttttttccctattgctaccttcctttgtggtttggtggttttctgtggtggtgtgctttgaatcctttcttttatagtatatgcgattaccattgtatttgctgtaaggctaacttaaaacattttatccttaggctactttaagcgataaaaacttgcctttagttgcatacaaaaactctacgctttcacaacccccccgcctttcgtgattttgatgtcaaagtttacacttttttaaatttgtatctcttattgtagctacagttacaattacttcttgtagctacaacttattgtagctacagttgtttttaatagtttcatcttttaaacctcctaataggaataacattgctttacctgccacctttacaatactagagaattctgactatgaattacttataccatttagttttttacttttatgtttctcattaactagcagtcttttatttaagcctaaagaactcccttttagtaattccagtagagcaggcctagtagtgacaaactcccttaggcattgtttatctggaaaagtgtttatttctccctttgccaagtaaagtattcttaattaacagctttgtttccttcagcactttgaatataccatcccactctctactggcctgtaaggcttttgctgataaagccactgaatctgtattggggctaccttgaatgtgatgtttcttatctttgctgctttcagtattctttgtctttgatttttgataacttggttgtgatgtgtcttggtgaactctttaggttgaatctgatcggtgacctcagcttcctgttcctgaattttgtcatcttttctcagatttgggaatttttcagctattacttccttaaatatgctttctaggcctttttctttcttttctccttaaggacctcctattatgcaaaagttagctagcttgatgtgtcctgtaattcttataggcattcttttttgtttttgtttctcattggataatttcaaatgtcttacctttgagctcattgattcttctgcttgatcaagtctgctgttgaagcgttctacttagattttcagttcagttactgtattctttatctttagaatttctattgtttttgtttatatttgttaaacttctcattctgttcagatgttgttttccaaatttcatttttctatccatattttcttgtactttgttgaacttaagaggattagtctaaattatttgtcatttcacaggtctccatttcttctgagtctgttactggagctttcatatccaatgtacaccagaaactttgctaattccacagtaaatgttagaaatgggtttttattgataaaatcagtggcttgctaattagtatgttagaagctggcttgcattggtgaagttgagggtattcgatatacgtatcccggctaaaatacttctaaagaacctctccagctttgcaagaatagagtataggaatctgaggaggcctttaacatggccctcagaaaggacacacagggcttgacctctaaacatataaacatatacagaaacgaggtcagaacatgttttgactgatgccagtagtaacacagcagagcctattgctggtcactgttaccaataccttgtgtaataaacctttcatttaaattgagaggttgtaagccccgaaaaagcaaagggcttacagtacctcttgcctgaacactcctgtgtgtgctttctaagaaatgcttttaaaataatgcctgtggcacaaattgatttgacactggctctctttataagctgctatcaaggttgtggggaatagctaactcttacccactttccctgacaaataggaacccgtagttacctaccaaaagtatgtaaaagctatatccgacccaaaggccagttaggtcaaaacaccttgaaaggagctcagtttaaggaaatcagtggtgtcacagtgccccactagcgtgtaaaagttttcatagtttgactatcggatgtgcttagtttcacagctgagtcttaccttgtgatacttttagagcaaaagtcaaatcaaagatgatttaaaaaaacatttcaaagattcataaagaataaatgcctcctcaggaagtcttctggagttctgcccacttcccttaggtggctctttactgtgcccatctcatagcctgtctccttccacttggtgtattgcagagtaaagctcactgtttacaggggtggtagaaagtgtggtcctttcccagactcctttttcccttcctcttctcatttttcagaagatgtgtttgataataaacgaaacaaaatgactaacacattgagctgagctacataagcagatgtcagtttgacgtgaagagtttaaaagatctatgcattatctggggactcctcccccagacctgaaggatcaggtgctgccttctatgccacctgtgcagacagcaaaagaggaaaaccatacccacgttcagtatgaacaaaggggacatttgaactctgtgtggacccctcattggaagggtgtttcttctcctgctgcatccacaaagagcactccttagccttgccttttgtcagttctctctccaataaggcttgagcagagacaacccagtgcagttcagagagactgaagtctggtgttccaggtctgagtcctagctctagctctctgtgtaactttgggatgtcccaaagtaacttttcacaacttgataggtgttaacttgaattttggatacaggtgactcttagcccatcccctctctgtgcttcagatatgtcatcacttgggccatatgacctctggacacctttcctactttccacaatttcagagcagcagagcagactggagctcctgctgcctctgagcttcagtgaattatcactcgttggagggaagcttcaagcattttgttatctttcaagagcaaacacagtgtctgtcagcaagaatatgtagcagatgctagtgaacagcagtgattagggttgaatgctggatttaaatatggagcttaggctgtgaaggaagcctgaagaacctagagccccatgaagctgccctctgtgagatctgacaccatttcccactgatgagccatgggtctaatgggtagcactgattccacatacagtatgtgagtgcaatacagtgaaagcaaagagaataaaatgatggctaacatgatgttccaaactttaaacaggagaaaaacacacaattccattatgtataagaacccacacagagatcaggagaataacctcattggagaatgaatgacctgtgtggggaatttagggtagagttgagattgaaaaaatgggccgaagtcaggtggccaagggccttaataccttgtatacaagatgtgtagtcaaggaagaccatgccttacttatgcatcaattcccttgggcctacaaagagctgcctagcctgggactgttgtagagaaaagctacagtgttccaatgacacagggactcctccatgtatgtatgagtgcccagctggctctgtaataaatcttatttttatttattaacttttcttgcgcattggcttgatgcatcagttggaagtcagaggccaaacgaagtgaacactgagccaaaggaagttctcagcctttagggaggcagaattcactttaaacacaataaacaaatgaactcacattatacaggagagagtcagaagatcccagtggctggtgtcatcgggccatatttgcccgaagtgcctattccttataggaacccactcccagggttgatgggctacatccttaggaggctttatgcctatgttctcctgaccactggctcctccagggctggccttttttagtctctctgtagaggttcctgtagctggttggatataggctttcacagaagggtcagtgccttgggtctggttacaggactgttaatcttgctttgttaagagtaatgttatttccccatttccaaattctccagggagatggaatgtcaaagatagtatgactgtagcacctaaatcctgggttccagaagcagagaagagaatacactgaagctgtagaaaggcctattagtccatgtcagagattaactggatgcgaggaccatttctgggatggtgtatacagaactggagaactggatagggagtcagaaacaaagagctgaagatgatgcctttgaagtttccaacgtggataactaggtcaacagaatattactcaagaaagtattaacataggattgagatgcagtcagtagtaatggaattgaaatttcaaagtatatacctcatggatctcagggggtgttgagctgatcacctgggctaacactcctatgaccctggggaaaatcaaatgacctggtactgtagccatggtaggggtgtcatcaccttaatccaactgggacagtgctgtttgatattcatctggaacttggtgcagacccacattttgctgggtttcaccacaaccaaggcttttttgattcttttctcttttaacatcagtacatcactgcaaagttaatcctcatataataggagatgaaactaattgcttataaaaacaagatttttacaacactaaaattgttcaagcatatgggcatatttatagttgcaggcagtgtttcagatgcagactgttcttggctgcagtggttgtttacaggcagcatctgttctgattaaatatttgatgattatccccgaatgttttaaagcatagtactgggctctgctgactgtacaacaaactggcatttttgacctatagggcactgggctaggagatacagttctgagggaagtgaaagatagttaacaactgcacaactgaccctttattagttgcaataaagcaatccaaccacccaacccactgtgatggctttcctactatttaaggttggtggtgtcaaagagacaccctcctgtacagtgtgcagtgaatcaacatcatttccacaaaacctccttcctgcacaaaggaattatcatactttgttacgaagtaaaattttcctgtatcagtcacaggagttcaccagttaagatactgttagttgaagacttctggggtgacttaatgaaatagctcagccatctggtttaaaaactggattcttctatccctccacacagctgtccatgcacctgcatcatctcaaggctggtctccctggtggtagaaaggctgacagtaaaaactggagccacatgattccttgcttaggcagtgtttcttcatactctcacatgagagcaggcatgttctttccctaggctcacagtaaacactctgtcaaatctcacaggcccaaagtgcttttggttatccccattccaagccaatctgtggcatggaagatagcattaccctgactgccttagactaatatacctactccacttctggggctgggaataattttgaggtcaaccatccaaactgcatgacagccattccatggaagaggtatggcctaaatctttggggcaacctgaattcatgaaaactctcttagatttatgtaactatttttagaattcacttctgtatcatttaattttactaataaaaataccacctttaccctaaatgtcagccaagcgtaaggctccgttgggacagaaggaactatcaaagctttgtgtttttatacattagcagcatttgacaaagaaataactctgaaggaaggagaataaccaggcagagtctagatgcatggaaaagaagtctttgagaaggcttcgcaggctgaaggaaagggcaggactacttcaggaatacaacgtttaagtaaaagaggttggggtctgttgatcttgaggagagatgaggatggactggaaaataggagtgagatatagtaggagaggaaaggatatggatgatgtatatactgtatgggtaaccatcagtcgaccctaagaagataatagttgttaaatggttagctatttaatgaaagaaacctgaacaagtaataatcttgagttgcaaagtggctggagtcacacaacagactaaatttctggtagaacaaatccagcagcttatgtgaaggttaccatgtctgaagctggatagaaaaggtctaacttccaaccaaagtcacagttcttgagctcggtacacagagacagactgctaacagctgatgtgtcctcagcgagagtgtcctcatttatatcctcgttctcttctgcctcctttttttgttttaaacttttgggaagtctcatcattcaatacagttctaatatatcacaataacagaggcggcccaatttctacaaattgactaattctatccctgaaaagttcatacaataaaactatacaaagcatcattttcaaccatcctatagaaaaatttccctattaaattttaactctaaatcctctatctctgttaataaccttactaaaaacttccccatcactgcctgccagggagatcaaaagaaaccaaatttaagaaacctccaacacctgtacctgactgaaaagcaaacatacagacctttcagtcctgcccctactattcagctcctatcacagagacatggtggatgtccccttgggaagcggatagctcttaggtggaagctaggcctgcaataagcaggccaggaaacatgtctcccaggccccatactcttggcagaagcacttggccaccagacagcatgtcctgtcaacctacagagttcttaaaaacaaacactgggacccagaatagtaccctgtggtcatagtgcccacagttcactaagcaccctcacaggtctttgacagaacactgactgccaggtcacctggtgggcagagaaatggaagattcccaggcccaactagcatctcagggaagaaccacaagcagagcaactttcagagctggtcggccagcgttggcacccagggaagcaagtgctattccatatttggagagaacataaaactcaaggaaacagagaagtcctactcaataccgttctcaactgaaaacaagagaacttgcaaaaaagaaacccagttttctggagtccatggagaaatatgaagccaatgctcggctaacagagcagaaagccttttataaataatgccagtcaaagctccaaaggtcagagctgatgcatgccggattgttctgacaatttcttaggtttctaggcaacaaggagctggtcaaacagctcacctccaaggacatactttataataccaccctggtagacgagagcgaggcagcagtgaaggcaatagtcaagaacaggagagggaggtaaagaacaagatgctcttcagacagtctggacaaagacagtccctacccagctctcaggagttgcctccttaagaaggtggaggcccaggcagctcccagggtgaccctaaagacagactccaaggaaaagggtgtgagggcaatggtgaatataaggaggtcccctttcagtaggcagagcaagtcagttcaggtcttatttttagagtctctgaacagtgaagagaagctttctgtggacagcattccaccaccatgggaggggaaaggtgccatgagagacttctccagtggggcatacaagcattgtgcagtgatccccaagatccagccactggcaggaaatcgaaaggcaagcttcttaaatacatagtatatggagacagaagtgtggagcactcccaaaatgaaaggccaagacccaggaacaacctccacaacctggagtatatacaaatgaacccagccctgctgactcagatccacaccatcctaaagcaggggtttctcatgaatgaaagggctaagtatttggtggagagaatgagaagtctcccactgcccacacattttttcttcagagatttcttttccaagagccccttgaataaaggaagggagggagcactgaatgccccagaaatcagaatgcatggtgcgggaagacgacaggaaatagttctcaaagagatcagaaaataaattggaagcaatttcattaccacaaagaacagacattttttctaaggcacccctcccctttccacccaattgtcattcagcaactactgaatacttaccaatgaaaaacgttacccctgacctcaaggctgctcctgagctctggtagaaaatgctttccttgtctataaaacatggcaagcaaggcaggatttaacagtaagggcacagtagcactgcaagccttcaaatggaaacctggagacaagcagtgagaacaggaagcaaaagcagggcaggtgaagctaggaccagggcatctggaactttccacacaggttggatctccatgccagacaacagttttcaaggaaaaatatctaagaggaacatgactttgggaaactttttggcagtactgcttactgtatactagagagtaaaagaatttggggaacattcaccaatttgcttcttcaggggcttgggtagggaacgtgaacaggaacctggctctaatttctgaacttttttatcagtaaaaacaatccaacaaacgaaagctagtcagtgagagaactgggagggtctgccctccttccctgagtcaagccttctggggggacctcctgacatttaattaagcaaagacaacgcccactgaaggaagctgacctgaaagtgacacgctactgtgaaatgagcatgaagtgggagcttgttacatatatgaaatggccagcgatcctgagcaaagcgcttcagagcttgagacctaagtcttctcatctatatactgagggctggacaagatgatctgtcaagccatttttatccctaatccaccaaaatccaatgctttagtttattgtcacaaaagcaggtatcgaatggctatcctgcagtgcctccaatcaacattcagactttttccctgaggcaatataagataacagttaacatgtttttatcaattaggtggtcatgagataaatatatatgggaagtggtagtttttcacttaaatgcatataataatggtacagctctctttgaatagtatttgtttatttcttaaatatttaagttcctaaagacggtaagaataacccaaggaagtgaaatcaatgtcacaaagcacatggctaaataactgcaggtttgcagtgccatgtgtgagatcagatgacagaagggagaactacctttaggcagaggcttctcatgtcccctggagtggccatgtgctgttctacatgactacttccacttcggttatgtagaagctatttaaagcacacagatgtttgtgatgagaaaaaagccacccttaattgaataatggaaattataagcatgatttgagggtgggggtggaggtgggagtagagatgggtagaaaggagtgcaatggaaacaaaggagccttcataaaattcaagtcacttcttaggataacgtgattgatttactcaccaccttcttaggaacataaagcaaacaagtgggttttccttttactgcttttctgaaatgagctacactcaagaaagcagcacgggggttgtgctgtccctgcacagtggcaggagagtatgaggagcaggtgaatgccacaacagctccatccaagatcatttttcacatgcaggaaccattcttatactaccctttactggtaatttctgtagaaatctggaagtctggttgacaccctcctgtacagtgtgcagtgaatcaacatcatttcccttggactttgcaatcacggtggcattcatacattcattcaacaagtatgtatgtacaggacagtggagtaaagaaaacagatagttcttactctcacgaggcttaaaatttcaggagggaactaggcagtcatgaagtaaacataaaaacacagattgtaataggcactagagaataatgaagactttggggaacacaatttaaattggaaaaatatctcagttccgttaacacatgttgaaggggaatagcattgttttgctggatttggggcctggatgcaagcatgttgggtatgcattgtagggtaatgcatttccttccatttgggcccaagtgtatatttaccacccagttgtgatgagctgggatcctcctgctcaatctcagcttgaagcacttggaggttatctgcctgctgtgggtgattattttggagcaaggtacttcatttgcctcaagaaacagatttgataccactactgtgcccttttggaacagagaagtaggcaagaccccagtgtgaggcagagtgatgggatctttagggacataattgatgatgtaactgatgatgattttggagtttatacatttccaaagtttcaaaatattttcacttggttgatttatctttatggtgatgacactacgtagatatatgcccttcttaaaagttacagtaagaggctgggagcggtggctcacgcctataatcccagcactttggaaggccgaggtgggcagatcatgaggtcaggagattgagaccattctggctaacacggtgaaactccgtctctactaaaaatacaaaaaaattagccgggcgtggtggcgggcgcctgtagtcccagctactcaggaagctgaggcaggagaatggcgtgaacccgggaggcggagcttgcagtgagctgagattccgccactgcactccagccagggcaatgagcgagactccgtctcaaaaaaaaaaaaaaaaaaaaaaaaaaagttacagtgagagttgacattgagaaaagggaggcccagccagggttatgcaaagacacagtagggaggaggatgggaagtttctgagacaccccagtaactgcatgggtccacaaagcacatgtacagtctgctctatgcactgcaggtacagccacgaataagacaaagtctctgccctcatggagcttggtgtctgtctactggagcagacaaaaacaaacctgctttttctcatttgcctctactgtgggttccgtgtataactctcaaatgctagcatttccatgcattccatccttgtccttctctcctctctgccttttccaggagaattttcctagccacaggttcagctatggtctatgtgctggagtcatacatttttatcttctgtgtacgtttttctctagacccatatttttaattgccttaggacagctctccctggatatccctcagacacaactaaaacatcattcaattgtactattaataattttcccttcaaaatccactcctcttctctatagacctaacagcaacactgtccgtccagctctcaaaacctgaaatgtgggagttgtctttgactcttatctttcccatgcatgagcactgaatcaatcagagtccctagcatgtcttggactgtggcctcccttctatcttcagattatctgcctttttagaattatggcaataacttaactgcaaagaccacatgtgcacttgtgctctttcagtttgtaataaatataaaatgaaaagtgattatgtctttgcttaacgtctgcaaaataaaagtccaaagtccttggtatggtctataaggccctcattatctgacctgcctgccttcccaatctcatcttaatcccttacagcctgacactcagacatactagactttccacctaactcactcacatacacctatcctgtatgtcaagattcggcttgaccttcatcacctacctatctgcagtatttttggatctgaattctctgcccacattgtactttcacatacttcttttacccttagtggtttgtacttatgcctggtctaactagattgtctccctgtaacagacttcttgattcaacaaagcagctctgaatcagcctgaaacctatggcacactgcaaaatggcaaacattcaataggtatttgcccagtaaatgttaatgaaaggaaaaaaattcaaacttcagttggaataggattagagacaagttaaaaaaaagtttcccatagaaatcttcctcatcgtaaatatcatcatcctaaatgtcccgagtctttccatgggtctaatttaccaaatcatgaagactctctttctcactgtgtatgtgtagtgggagaggcagagacagaacggtttttttttttttttgcaagtctgctctctggattcgtttttctggggatgaaatctcaggctatgtagcttttctggtccctttttggtaatcaacaaatcatcagtttctctaggtattaaaaagcctacacttttgaaacaccaagaggccaaactctctcttaattgaaaacataattctgcctcttactgaggtctttgggtgggaagcttaaagacagcagtgttggggcaattggtttttcttttcctccctccaccttctttcccattcaagagctgttgctgccctttctggaggggagggaattagaaaaaaagatcctgccttacccagtcactgttaattattactttgagccagggtggggaatgactttctttcctggaatacaccctgctggaaaccacagctgagattctctaagctggcagcttccaacctctcctccctcaacagcttcaacattatatggcactcagctgcagggtgcctggctccaggccggcagtccatttgtgcagggtagttttcaagatggctcatagcccatctctttcagtgaccagcgtggcctatgggaaacatacatcttgactccattattggtaggagtactctttagttaactgccacaggccagttcagacacgtcttaccctgagagccttttcagagtcaggtaccatccctgtgtcccccagcatctgaagatcacaggtgaaggtctccaagtagttcacttaaaaaatacctcagtaggtttaagaacagggaaagctcccaattcattctgtcaggagtatgactctcatccccaaactggacagatatataatatgaaaactacagaccaatattccttatgaatatagatgcaaacattctaaacaaaatactagcaaaccaaatccagcagcatagaacaaggtttatgtagcatggacaactgagattatcccaggaatgcaaagttaattcaatatacaaaagtccattaatacaacatattaataaaggacaaaacaacatgaaaatcttgatacagaagaagcatttaacaaaacccacagcccctcctttttttttttttgattaaaaaacactcagtaaactaggtttaataaaagaatttcctcaacttgaagcaaacccacagctagctaacatcatactcaatggtgaaagactgaatgctttccccttaaaatcaggaacaagaaaaagatgtctgctcttgtcacttctatccaatattgtactggaggtgctagccagcacactaaggcaagaaaatgaaataataggaatctagactggaaaggaagaagtaaaagtatttctgttcacagatgcatgatctcatatgcaaaaaatactaagtgacaaaaaaagaattttagaaaagctacaatatacaaaatcaatatacaaaaattaattttatttctaacaccagtgatgaatacaaaaataaaaattagaaaataatcatataattaaagtggcatcagtaaaatacttagaaaaaaattaaagacgtcaagacttgcacactgaaatctctaaaacatcactgaaataaagacctaagcaaatgcaaagacatcccacagtcatggaacagaaaacctaacaccattaaaatagcagttatttctcaaatttatctaccaattcaactaaatccctatcaagatcccagctgttttgcaggaattgacacaatgatcatataaaaattcatatgcaatgcaagggacccaaaacagacaaaatgattttggggaaaaaaaaaaaaaaaatggaggacttgcacatcccaaatctaaacttactaccaagctacagccatcaagacagtgcggtgctggtataacgacagacatatagggcaatggagtaagactgagaatccagaaagtcttatatttatggtcaactgttctttgacaagggtaccaggaccattcatggggaaataatagtcttttcaacaactggtgcttgggcagatggatatacagataccaatgcacttatgcaaaaaatggatgaaataggaaacttcactacattctactgcatgctcggtcattttcaatcatttaggtggcaacactgacaagataacagaaagatggaggtaataacatgtgaaaggcaaaatggtttgtttttttttttaaaaatgacagtctctatcatgaatttacttacactccaggcaaaggttattagaagaaaaaaagatgtaagaaaattccttaactgaaatgtggaaagagtatcaagaggagaccctaagacactctgtaagaatcccagtgactcctcactgttcaactaagaaatgtaccccattatgctgtgctaccacggaagcattggaggcactttgggggttgatgaagtcttcatggatgaggtactttaaatatctggtcatgaagagtatttgagaaatatgacaagtgaagatgctgggtaggaatgcagcgaggaaagtgtgtaacacaaggccaaattggaaaggtcagtgagggggcaatctgtggaggcactgaatgcagaggatattaaaattagcaagatagtgttctagcacaatgaaaaggattggaagagggagatgagagtcagggagtgaaattaggcagctgctaagaatgtccgggtgagtcagaggatcaggacctgtaagggcagtataaagaaggaaggaatgatgggagaggtttagggggaaagaagtgacaacctgtgacaattgaggaatgagagattttgatgattggggtgaaggttacatttcttaaaaagaaacaaagaatggtcggtcgatagagatgcaagatgaagaattttgtttttaggactactgactatgaggtaacaaagaaatcccagagacagaggtctgagcaaaagatatgggattggggaagaatctttgggagtgactgaggttgactagagggaactgggcaagaacaggtaaggactttaagcagaattggaggagtatccatctaaaatctgggtaaactgggatggaaaaacaagtagccagagaagcaacagcccaagctagggtgttgtcaaggttatagatgttactgattttggcaatgaggagattataaggatccttgaagactgtactttcggtgaaactaccatgcattagtagcctttcacagtgatatctaccccacacctgcatcaaaatcttgcagtgcctattaataaatgccaacccactaacggggaggggagaaaagacttgggactctgtacttgtaaaaccctctaccctccccaggcaattctttacacactaacactgttgaggtaaaaggagacaagtgagggagcaaatggaaggtgtgtttttggattatacagtggctcatggaagggtgggaggggtacagatggcccttagacactggcggaaagtcagagaaaaaaaattacagtaagcaggtaaagggatcaagactacacagggactagtcttgggacgacatttcttcctctgacagtttgtatggaattcttgagaaaaattcctcgaaggggcctgaaatctcagaatggtcatgtttttaatgggaatagggagtgatgctgccccattacaaccatctgttctaacagaatgtctgtaccgaggagggatgagtaacatcggcaagttctgttcgaagcctttttcaagtttctttttgatatgtatctatctatctatcatctccctatacaagcaagcatccccaaaagtagttgtctcaggaaaccagggttaggatgaccagctcatttgctggagctgccaaggtccagaaaagtttggctgcaggctgttttcatgttttatgtatgtttgaaatgttctataataataaaaggttaaaaaagtttacatttatttggaaaaccagttactttagtttatggttcctttttttccctccagagcttcctggagattgaggtctaattcaaagaaaaccaaaatatataatagagtacctgggcaaaaaaagtacttttataacataacatttggggtagaggaagtatccactgtagtcaaaatgtctatgttttgctcttccttattgttcagggacattccattaaatagtaatgaaaaggcagcaaaagtaagaggagtgacaacatgcccggcataattaagcaagctagagcagctattctgtgcaaccgacgattttttttctctaaaattttaagggtaggttcattctgactctgttaaaagtctacttgatgtgaacaactctatatctgataacctatttcaattaccactttaaaacttgtcatatggatacgttattacaattgtagaactttaataaataccataataataaaacttgagaactgaagagcacacatttcttcacgaatttattatataaaacgccctcagagtatttaatttctcctcactttaattacacattaagaagcacagtggatgagaagcctttaagatgactacagttgcacgaaggtccctttcatcaaggtagcgtatgtaccctaacagtgttctaaaggctggcccagaaaaaccccatgttaccttatcacaatatggaaagcattgtcttctttttccactaaattaaattatggtgaaaagtgccacagttttatttagcattatggtacataacaaacagttctgtctcaattatgaaaaaaattaattaaaataatcctgaaagacatcctttttctccccccaatgatttgaaagctgcatttttcctgccaatttcaaacaaacaaatcatcaggttgatctacagtaatcagttaaaacaatcagtcaatcaatcaatcaatcaccaaggcacaagctcagcacattagctatagcttgtagcaaaaggatatatcaatgtctcaccttagttaaaaatacataatccttttattttataatgcaataaaagaaattaacaacatcacatacacagaagactaggaaaggggaaactacttacttctggaaatcagtaatgtaaacctacttgtacttttccatagtacatgaaagtaacgtttaacatgttttgaattaattaattaaatttaatctgtggggctatacaatgtaattcttaggagtaatagtttcattcatttccaggtcagcttactgtatgattaagtaacacaaggcacagtagccatctttttcattatgttgcaacactgatcacgtgcctcgataaaatggctgattcaacaagatgatggcaacacgaaggggagactttggattgtctatttaaaatctaggtaataagtaagtaattaataaaaactctatcttaagtgcactttcacatgctttttgtttataataaacaaacaacaaacttcctaactttgttgcaataggcttgactaccatttcatttggccaaatgcactttccccagtaaacttaaaacaacaacgagaacaacaagaacaaaaatccctgtcctttcatatactaagaaagaggattggctactgaaacagttcattgcaagacacatgaagacgacatactgtggcatgagttgtttttgtttttaatttgttgtgctgttactaaagttctgagggctgcagttaaaacattccaatttctcccttccttccatctttctttattgattgattctcaagattttgcacagaaaactctttgggggctagaacagcagtaattgcatcacactgttttcaagacttcaagtttcaaaagcaaatcattaaaaaaaatacagttcctgatttgagttagatacagggacaaaaaagtagcacatacttgaaggttacgtggtctacaaatggtggcaatattttccttgggagagtagttctgttggtatatattttttaaatactcaaaaggctcaacctcaagcagtaataaacacaagcaaaagtgatttaacccttaaaataaatattcagaaaaacctctctgtacatacaagtgaaagaatatgtaacactttcacgcaaaaaaataattataataataataaaggatttgttcatatatgtagctgaaatctgctgttccagcccacatgtccccaataaagaagggaggcacagacataggtgactactgtggttgactatcttacagcctttttgtactgggacactatcaccaccaaaaatttatccctcgttatatttttaaaattttttaaattttttctttttttttccttccttttttttgttttattttgttttgttttgttttacagcatgccaaatcctttggcatacgtgatggccttcaacaatctctctttaagtttttctttgcttgagtattccggaagtaaaagcacattaaagcaagtatgagatgtaggtaacctaaatagagaaaaggggaaaaaaacaggaaaactgtaagtcatgggaaatacacttagaattaaatgctcctatttttagattgtatatagttgagacggtctgcaatgcaaactatacattaatgcaaatcataaactttttgttgtgtaactaccaagttgcctttatcctataaattactcaaagctagtgacgatgataagatactgtatccattgagtttttactacataacagataccattttaggtactgaattcttacagttcatttaactaaatctttccacaacaaaaccacagagaggacatcagtaaatgcactttagaggttaggtcactgagaagtcaagtaacttcctctaaggtggagaaaatactcaaacctgttttacaagactgcaaagtgtgtgctcttaaatgcttattagaaacactgctggcaatatgactaagaaaatgatttgataacaggattctagcacaatcaaatgataatcttccgagcctcaatgtaaccattctaaatagatgatcatgttatatggctttcaattaacaagctgggaatcaaaaaagtaaatgaatcacactaatttgattccaaaccaatgtgagccccataataatttttaactagggcaatttcttaaaagtttcctcacacaatgacagcaaagtattttctcaattgtctaatatgatttggggatttgtatataaaatcacgaatgtgctcagaaactataaagacagttcatatgtatgtgacgaggaatgcaaggttttcggtaggtatacagtcacaagttaataattacctacctttctgtgtctgggccatttttggctataatcatctttaattttcctagtcctcccacaggtgctctgtctgtgcccgttgtaaactgcaagaagagtcttttctgttcatctgtaaatgaatgaacgatttcccagaactccctaatgagaaaaaatacaatactggtttcagtttggcattcattatgactggtactaacataagcttatgatttgcattaaaactatattaagagacaacttggaagttaattatcaggatagtatcacttctggtgatttaaaaatttccaagcaaatttatcctgaacagctctgaatacgtaaaaatttagattagattacaatatagtaaaatattagtactacaatagtaaaaaattgagaaaacccaagtgtgtagtaacaggaagtgactatttaaactatggtataatcacattatgcagtcagtaatgagcaaaagacaaaatcctatgaattgaaaaagactgaaatgaatatttggaaaaattaactccaggtgcctttgggtatatattttatttatcctttctcctttattctcctggtctactcatcctttaactcaactttgggaggaaaaagtgatacagattaaggacaaaagaaaaaaagaccccctgccccaaccaactggccccaaatgcaaacaactacatacctaatagcagtaatacaaaagttcaatttttatatgaactagaatactttaaacaagtaatatgtgctgtgtatggaggaggagaattattctgacatttctgccacctgcagttattttaagagaatgatttatcctgtggcattcctaaaatctatgtaataaaagctatgttttaatgacacttatgttagtttgagttctaaaaaacgaaatacaagctcataaagtgcaatcttgaagtttatttgaataatcaggcatctataaaacatatatacactagttcatagctaaataaatttttttttttttgagacagagtcttgctctgtcgctcaggctggagtgcagtggcgcgatctcggctcattgcaagctccgcctcctgggttcgcgccattgtcctgcctcagcctcccaagtggctgggactataggtgcctgccaccacgcctggctaattttctgtacgtttaagtagaggcaggatttcaccatgttagccaggatgggtctcgatctcctgacctcgtgatccgcccaccttggcctcccaaagtgctgggattgcaggcatgagccaccgcgcctggcctatagctaaataatgttaagattaaaaaattaaaaaaaaaattaaaagtatttttagttgcttatataatataaaatgcattttaatagttatttagaaagttctttccagagccaggtgtggtggctcatgcctgtaatcccagcactttgggaggctgaggccagtggatcacctgaggtcaggggttcaagatcagcctgaccaacgtggtgaaaccctgtctctactaaaatacaaaaattagccgggcgtggtggcagatgcctgtaatctcagctacttgggaggccaaggcagaagaattgcttgaacatgggaggcggaggctgcaatgagctgagatcacaccattgcactctagcctgggaaacattttgagccatctcaaaaaaaaaaaaaaatttcatctcaaaaaaaaaaaaaaaaaaaattccagaatgttagcagagaacatcacacatcccaaaccagtaattacaattttctaccttttgatattctaatatcccaacatactatttttattaccaaaatgctggcatttttggtgctgcaacaccatttatctgaaataacttaaaagttttattagaattcttggctttctccaatctcttgccacttcccttccctgcccctttacaaatccaccagtcagcaagagaaaacaaaaaagaaacacacaacaacagaaaactggacatgaaacttgcaggagcatctctcaggtaaggaggagaggaatcggaccccagttcatgatactccttttggggaggctgacaagagaaaacaagtctgctgtagcacagaaccctccaacatagcagaaaagctgccctgcagtgggtggtaataaagcctcctcacactgcaggagggctctgaagcttaactgaaccagctacacctaaggaggtagaccaaagagcaccaccgaagaacagagacagaccctgcaggaagtggcagatcagcagtggtcacaatgaccagtctggctatattattacggagctgtttctttggacttttaaacgaaacctttaaaaattttatacaatgaaaacagggaacaaaatgcatctctattcctataagtgttatgtgtgttacattaacattttgaattaaacaagaatgcatatatttagaaagctgaagaaaacacgaagaagcttccaggtttccacataaaagtggtggctgtatcgatcacatcctactcacatctctaaaaatctacccggatcaaaaaggggaaaataaaaaaaaaacctatgacttaggtctagagtaaaactaggagacagaacaataaactccaaataccaaagaagtggggaaatgagcagggtccagcaggagctacatcgaagagtggcttgtgcggaatcatactgattagggatcacccaaggccagaccccaccccctccaccacctgcccctcaacagagcactactgagtgtaggttagagcactggcaacagggtggttaaaggaaggactacagaaaagtacttgggtccagcaatggcaggctcaggaaggcaccatgcatgaaaggaaggggggtaccttcaaaagcagaagtgtcccttaagcggctgtaaaggggtggaagcagctaatgaaaaagagtcttcattaagctacatggagctgaaaaatcagaaagtggtgattctgcccttactaaagcaacagaagagggatcccttcaaccaagacccctaaagccacacaattcctccctgctccaccatgaggtctagtaataacaggtccagaaaaaagtaacatctgttatagaaacataaacaagaaaaacagaattcggaagtctaaataaagttattatgggaagagtctggcgaatgagaagcaaaactttccggtagacaaaagtagaccagaaaaattcagtcataaacaatgggaaactagtaacaccacattccaacacaaaaggagaatgcagcagcagacaagacagaccaccagtgatgagaaatacaaattcagaaagaaatgagcacatcttaaataaaaataagaatactaaaaaatactgaagaaatatgcaaggtaactttggaaataaaggctaattttattataaggtgaccaaagaagaagcagtatgactaaaatttctactgaggacagactctagaaaattaaaaaacaaagaaaatgaataaaataaacataactaaagaaatgcatatcaaagcataataacgaaaaagattaatagctaactacataaacataaaacctttcatttggcaaaaaaatcctataagcaaggttaaaagacaaatgacaaactgggaaaaaatatttgcgaatttaccagcgttaaaggactaatctccttaacatataaagtttctaaaagtgaagaaaacgaccagctgagcagcaaaatgagcaaaagactatataaaactataaacagctctacaagaaaaaatgtccattatcattcataattgaggtaagctcaaaaattgtaactatagtaaaataccatttctcaattggcaaataagtaaacatccacatttaacaacacattctgtttggaaagttttaagaaaagagatactgttgtaaactgctggtgggaatgcaaaatgttatttttctgtgaaggaggatttggcagtatctagcaaaattacacatgcatctaccatttgatgcaacaatcccacttctaacaatttatcttaatgatactttacatatgtacggaataatgcatgaacattaagaggattcactgtgacattagtactaacagcaaaaggttaaaaataacctcgatgcccataaatattgataagctatgctgcatccacacaaaggagcagtattcagtcatacaaaacaaaatggaaaacacctctgcaataatgtgggatgattctcaggatacactaggaagattattttcctagttctgtcctctgataaggcctacaagcagtaacgttcaaaagcaaaggccatactttgcatctaaaatctgacttctaaatgccattctccaacaatttattggaaaaataacttattccaggcctgagaatgaatgttcgagattagttagaaatctcaaaatctaatagggtcattttaaaagcacacgatagctaagcaatttgaatatcatttagagtaatgactgtgaaacgtattaaatacaaaaacattcattagttcgtaatattcaaaaaggcagcaaaaaccaaccaaaaaacaaaattaaattgtcactattaaaattattacattaactccttactctaaaaaccttaaatctattttatcatgccttttctgtagaactgtttttcaaggtaatcaaatggcacccaatgatgagaaaaaagaatgccaggtatatatgtaggacaagcagatggaaattttttttccccaaacagccattttgcaatccccaatgagataacagattaaggtaatgatactgataaatacgaaaatcaggtgaagggcaggtagcaggttctggaaagatgatgatggcaacgacatagttattattattattattttttgttttattccctcccaacctcccctacaaaaacagcaaactgaatgagaaaaccaaagacccagagacattatctacaacaaaaccatgaaccattgcatataattggacagaaacaaagctctgacaactacaagactggttagtaaggaagcagatgcaaggtaactgactgggtttctgacagccctgagaacactgccaacccactggaaagcacaggccaatctgaaaacagggcttaaagttttaaaaagttctacaggatctaatttgcagatgattacaaagggatcatgatgtagtaagctctgggcccttaaaacacccgaataccaaacccccaccagaaacaaaccttgtaccgaggaaaaacttctgggaataacttccgaatggaccaggtcagtaataaaaaccaaaaaaaagttcaaatatatgtgtgggatagaggagtaaagaaggcagtttcagaaagcacaagggcatatttttgaccattttacaaaaacacagacttctcctcgtccctaaaaagcgaaaaaagttatcctggcccatctctccctcgtccaagtatgagaaactcatttcactcacacacacacacacacacacacaaataaataaataacagaacagagtgaaacagtgtaattattagagaaaaagtatgtgcatatacatgagaatggtgtccctacagaaaatcaaagtacatgaaacaatatgcaaataaaacatgaaaactgtaaacaaatttcaaactgagctaaaaagaatttaaaaaataacaaatcattagaaatgggaaatttctgaatgagggtaactgaaaaaaaggaagacatgacacaactaaggagtaattagaaatgcaaggaaaaaaatcccatcaaatacaaagaaactaaaactaaactagaagaaacataaaggtgaataaaacagaacaataataccagaacctgaaggcagaagaaaaatcttaaaatcaaaaagaaacaaacccatgcttgagactctccttgctggtccagagccacagtgctgctgtgtactggcaggaggaattctgctataattggtcctggcagtgttcacctttcctgcaagtgttccacagcccagggacacaatgtggtcaggagcactgccaggaacaccagcaagggggagcctgccacaacaggcacaaggctcaggaagcactctccagcccacgaaaatctagtgggggtcctcttccctcacccaaacacactctgcgcagct

SEQ ID NO: 2 - DNA, 48 bp, synthetic construct
  misc_feature 1..48: note = Synthetic
  source 1..48: mol_type = other DNA
tgctcttctttctactttattctcgagaataaagtagaaagaagagca

SEQ ID NO: 3 - DNA, 41 bp, synthetic construct
  misc_feature 1..41: note = Synthetic
  source 1..41: mol_type = other DNA
gaaataataatctgatgaggccgaaaggccgaaactaaaag

SEQ ID NO: 4 - DNA, 41 bp, synthetic construct
  misc_feature 1..41: note = Synthetic
  source 1..41: mol_type = other DNA
gaaagaagctgatgaggccgaaaggccgaaaaataataatg

SEQ ID NO: 5 - DNA, 21 bp, synthetic construct
  misc_feature 1..21: note = Synthetic
  source 1..21: mol_type = other DNA
tgctcttctttctactttatt

SEQ ID NO: 6 - DNA, 20 bp, synthetic construct
  misc_feature 1..20: note = Synthetic
  source 1..20: mol_type = other DNA
cttttagtcattattatttc

SEQ ID NO: 7 - DNA, 20 bp, synthetic construct
  misc_feature 1..20: note = Synthetic
  source 1..20: mol_type = other DNA
cattattatttccttctttc

SEQ ID NO: 8 - DNA, 67 bp, synthetic construct
  misc_feature 1..67: note = Synthetic
  source 1..67: mol_type = other DNA
tcctgaataaagtagagaagagcagtcagtcagtggccaaaactgctcttctttctactttattcag

SEQ ID NO: 9 - DNA, 21 bp, synthetic construct
  misc_feature 1..21: note = Synthetic
  source 1..21: mol_type = other DNA
tgtggagaatcattgatatat

SEQ ID NO: 10 - DNA, 21 bp, synthetic construct
  misc_feature 1..21: note = Synthetic
  source 1..21: mol_type = other DNA
gtggagaatcattgatatatt

SEQ ID NO: 11 - DNA, 21 bp, synthetic construct
  misc_feature 1..21: note = Synthetic
  source 1..21: mol_type = other DNA
gtatatgagagttccattatt

SEQ ID NO: 12 - DNA, 21 bp, synthetic construct
  misc_feature 1..21: note = Synthetic
  source 1..21: mol_type = other DNA
tggctgtggaaacgcttattt

SEQ ID NO: 13 - DNA, 21 bp, synthetic construct
  misc_feature 1..21: note = Synthetic
  source 1..21: mol_type = other DNA
tggatcgatgatgagaataat

SEQ ID NO: 14 - DNA, 21 bp, synthetic construct
  misc_feature 1..21: note = Synthetic
  source 1..21: mol_type = other DNA
ggatcgatgatgagaataatt

SEQ ID NO: 15 - DNA, 21 bp, synthetic construct
  misc_feature 1..21: note = Synthetic
  source 1..21: mol_type = other DNA
gccctccaataggacaaataa

SEQ ID NO: 16 - DNA, 21 bp, synthetic construct
  misc_feature 1..21: note = Synthetic
  source 1..21: mol_type = other DNA
tgacccaagacttgctttaat

SEQ ID NO: 17 - DNA, 21 bp, synthetic construct
  misc_feature 1..21: note = Synthetic
  source 1..21: mol_type = other DNA
gacccaagacttgctttaatt

SEQ ID NO: 18 - DNA, 21 bp, synthetic construct
  misc_feature 1..21: note = Synthetic
  source 1..21: mol_type = other DNA
tgtgctgaaagaaggaaatat

SEQ ID NO: 19 - DNA, 21 bp, synthetic construct
  misc_feature 1..21: note = Synthetic
  source 1..21: mol_type = other DNA
atatggcatgcctctattaaa

SEQ ID NO: 20 - DNA, 21 bp, synthetic construct
  misc_feature 1..21: note = Synthetic
  source 1..21: mol_type = other DNA
tatggcatgcctctattaaat

SEQ ID NO: 21 - DNA, 21 bp, synthetic construct
  misc_feature 1..21: note = Synthetic
  source 1..21: mol_type = other DNA
atggcatgcctctattaaata

SEQ ID NO: 22 - DNA, 21 bp, synthetic construct
  misc_feature 1..21: note = Synthetic
  source 1..21: mol_type = other DNA
tggcatgcctctattaaataa

SEQ ID NO: 23 - DNA, 21 bp, synthetic construct
  misc_feature 1..21: note = Synthetic
  source 1..21: mol_type = other DNA
ggcatgcctctattaaataat

SEQ ID NO: 24 - DNA, 21 bp, synthetic construct
  misc_feature 1..21: note = Synthetic
  source 1..21: mol_type = other DNA
gacagtggaaccaagtttatt

SEQ ID NO: 25 - DNA, 21 bp, synthetic construct
  misc_feature 1..21: note = Synthetic
  source 1..21: mol_type = other DNA
gagactccatggttcataata

SEQ ID NO: 26 - DNA, 21 bp, synthetic construct
  misc_feature 1..21: note = Synthetic
  source 1..21: mol_type = other DNA
agactccatggttcataatat

SEQ ID NO: 27 - DNA, 21 bp, synthetic construct
  misc_feature 1..21: note = Synthetic
  source 1..21: mol_type = other DNA
ttgtggtcatactacattata

SEQ ID NO: 28 - DNA, 21 bp, synthetic construct
  misc_feature 1..21: note = Synthetic
  source 1..21: mol_type = other DNA
tgtggtcatactacattatat

SEQ ID NO: 29 - DNA, 21 bp, synthetic construct
  misc_feature 1..21: note = Synthetic
  source 1..21: mol_type = other DNA
gtggtcatactacattatatt

SEQ ID NO: 30 - DNA, 21 bp, synthetic construct
  misc_feature 1..21: note = Synthetic
  source 1..21: mol_type = other DNA
ttgcaacaaggtaagttatat

SEQ ID NO: 31 - DNA, 21 bp, synthetic construct
  misc_feature 1..21: note = Synthetic
  source 1..21: mol_type = other DNA
tgcaacaaggtaagttatata

SEQ ID NO: 32 - DNA, 21 bp, synthetic construct
  misc_feature 1..21: note = Synthetic
  source 1..21: mol_type = other DNA
ttgattgatgccctcataata

SEQ ID NO: 33 - DNA, 21 bp, synthetic construct
  misc_feature 1..21: note = Synthetic
  source 1..21: mol_type = other DNA
tgaagttgggtaaagtataaa

SEQ ID NO: 34 - DNA, 21 bp, synthetic construct
  misc_feature 1..21: note = Synthetic
  source 1..21: mol_type = other DNA
gtcaatacacaactgataaat

SEQ ID NO: 35 - DNA, 21 bp, synthetic construct
  misc_feature 1..21: note = Synthetic
  source 1..21: mol_type = other DNA
caaagagtttcagccataaat

SEQ ID NO: 36 - DNA, 21 bp, synthetic construct
  misc_feature 1..21: note = Synthetic
  source 1..21: mol_type = other DNA
catgctttccagaggaaatat

SEQ ID NO: 37 - DNA, 21 bp, synthetic construct
  misc_feature 1..21: note = Synthetic
  source 1..21: mol_type = other DNA
tccagaggaaatatgtttaat

SEQ ID NO: 38 - DNA, 21 bp, synthetic construct
  misc_feature 1..21: note = Synthetic
  source 1..21: mol_type = other DNA
tttaatcatctgccctatatt

SEQ ID NO: 39 - DNA, 21 bp, synthetic construct
  misc_feature 1..21: note = Synthetic
  source 1..21: mol_type = other DNA
ttaatcatctgccctatatta

SEQ ID NO: 40 - DNA, 21 bp, synthetic construct
  misc_feature 1..21: note = Synthetic
  source 1..21: mol_type = other DNA
tgcaagataccatacatttat

SEQ ID NO: 41 - DNA, 21 bp, synthetic construct
  misc_feature 1..21: note = Synthetic
  source 1..21: mol_type = other DNA
gcaagataccatacatttata

SEQ ID NO: 42 - DNA, 21 bp, synthetic construct
  misc_feature 1..21: note = Synthetic
  source 1..21: mol_type = other DNA
ggtccatgtgatttctttaat

SEQ ID NO: 43 - DNA, 21 bp, synthetic construct
  misc_feature 1..21: note = Synthetic
  source 1..21: mol_type = other DNA
tgccataactatagcatttat

SEQ ID NO: 44 - DNA, 21 bp, synthetic construct
  misc_feature 1..21: note = Synthetic
  source 1..21: mol_type = other DNA
ggataccatccacactataaa

SEQ ID NO: 45 - DNA, 21 bp, synthetic construct
  misc_feature 1..21: note = Synthetic
  source 1..21: mol_type = other DNA
tgtgtttccttccagataatt

SEQ ID NO: 46 - DNA, 21 bp, synthetic construct
  misc_feature 1..21: note = Synthetic
  source 1..21: mol_type = other DNA
gtgtttccttccagataattt

SEQ ID NO: 47 - DNA, 21 bp, synthetic construct
  misc_feature 1..21: note = Synthetic
  source 1..21: mol_type = other DNA
gtgtgctgaaagggctataaa

SEQ ID NO: 48 - DNA, 21 bp, synthetic construct
  misc_feature 1..21: note = Synthetic
  source 1..21: mol_type = other DNA
ttgaaccaaccaccctataaa

SEQ ID NO: 49 - DNA, 21 bp, synthetic construct
  misc_feature 1..21: note = Synthetic
  source 1..21: mol_type = other DNA
taaacgttgtatggcttattt

SEQ ID NO: 50 - DNA, 21 bp, synthetic construct
  misc_feature 1..21: note = Synthetic
  source 1..21: mol_type = other DNA
atccctgtgtacacaatattt

SEQ ID NO: 51 - DNA, 21 bp, synthetic construct
  misc_feature 1..21: note = Synthetic
  source 1..21: mol_type = other DNA
atgtgtggtaaatccattatt

SEQ ID NO: 52 - DNA, 21 bp, synthetic construct
  misc_feature 1..21: note = Synthetic
  source 1..21: mol_type = other DNA
tgtgtggtaaatccattattt

SEQ ID NO: 53 - DNA, 21 bp, synthetic construct
  misc_feature 1..21: note = Synthetic
  source 1..21: mol_type = other DNA
atgagcatttggcagtaatat

SEQ ID NO: 54 - DNA, 21 bp, synthetic construct
  misc_feature 1..21: note = Synthetic
  source 1..21: mol_type = other DNA
tgagcatttggcagtaatatt

SEQ ID NO: 55 - DNA, 21 bp, synthetic construct
  misc_feature 1..21: note = Synthetic
  source 1..21: mol_type = other DNA
gagcatttggcagtaatatta

SEQ ID NO: 56 - DNA, 21 bp, synthetic construct
  misc_feature 1..21: note = Synthetic
  source 1..21: mol_type = other DNA
agcatttggcagtaatattat

SEQ ID NO: 57 - DNA, 21 bp, synthetic construct
  misc_feature 1..21: note = Synthetic
  source 1..21: mol_type = other DNA
gcatttggcagtaatattatt

SEQ ID NO: 58 - DNA, 21 bp, synthetic construct
  misc_feature 1..21: note = Synthetic
  source 1..21: mol_type = other DNA
gagactctgctgaagtttata

SEQ ID NO: 59 - DNA, 21 bp, synthetic construct
  misc_feature 1..21: note = Synthetic
  source 1..21: mol_type = other DNA
agactctgctgaagtttataa

SEQ ID NO: 60 - DNA, 21 bp, synthetic construct
  misc_feature 1..21: note = Synthetic
  source 1..21: mol_type = other DNA
cagttatagaagtgctaattt

SEQ ID NO: 61 - DNA, 21 bp, synthetic construct
  misc_feature 1..21: note = Synthetic
  source 1..21: mol_type = other DNA
tatgatgacctcctgaattat

SEQ ID NO: 62 - DNA, 21 bp, synthetic construct
  misc_feature 1..21: note = Synthetic
  source 1..21: mol_type = other DNA
gggtctggtgtttcatttatt

SEQ ID NO: 63 - DNA, 21 bp, synthetic construct
  misc_feature 1..21: note = Synthetic
  source 1..21: mol_type = other DNA
ggtctggtgtttcatttattt

SEQ ID NO: 64 - DNA, 21 bp, synthetic construct
  misc_feature 1..21: note = Synthetic
  source 1..21: mol_type = other DNA
gggtctcattatagttaatat

SEQ ID NO: 65 - DNA, 21 bp, synthetic construct
  misc_feature 1..21: note = Synthetic
  source 1..21: mol_type = other DNA
taacagcatgtcaggtaataa

SEQ ID NO: 66 - DNA, 21 bp, synthetic construct
  misc_feature 1..21: note = Synthetic
  source 1..21: mol_type = other DNA
gcagagccctattcctttaaa

SEQ ID NO: 67 - DNA, 21 bp, synthetic construct
  misc_feature 1..21: note = Synthetic
  source 1..21: mol_type = other DNA
ctcctaaatgtttggaaattt

SEQ ID NO: 68 - DNA, 21 bp, synthetic construct
  misc_feature 1..21: note = Synthetic
  source 1..21: mol_type = other DNA
ttagcatttctgacctattta

SEQ ID NO: 69 - DNA, 21 bp, synthetic construct
  misc_feature 1..21: note = Synthetic
  source 1..21: mol_type = other DNA
tagcatttctgacctatttat

SEQ ID NO: 70 - DNA, 21 bp, synthetic construct
  misc_feature 1..21: note = Synthetic
  source 1..21: mol_type = other DNA
agcatttctgacctatttatt

SEQ ID NO: 71 - DNA, 21 bp, synthetic construct
  misc_feature 1..21: note = Synthetic
  source 1..21: mol_type = other DNA
acaggatatagggaataattt

SEQ ID NO: 72 - DNA, 21 bp, synthetic construct
  misc_feature 1..21: note = Synthetic
  source 1..21: mol_type = other DNA
caggatatagggaataattta

SEQ ID NO: 73 - DNA, 21 bp, synthetic construct
  misc_feature 1..21: note = Synthetic
  source 1..21: mol_type = other DNA
tagcactgaaatgcctatatt

SEQ ID NO: 74 - DNA, 21 bp, synthetic construct
  misc_feature 1..21: note = Synthetic
  source 1..21: mol_type = other DNA
agcactgaaatgcctatatta

SEQ ID NO: 75 - DNA, 21 bp, synthetic construct
  misc_feature 1..21: note = Synthetic
  source 1..21: mol_type = other DNA
gagcagaaatcaatgaaatat

SEQ ID NO: 76 - DNA, 21 bp, synthetic construct
  misc_feature 1..21: note = Synthetic
  source 1..21: mol_type = other DNA
acttgtaaccagaccaattta

SEQ ID NO: 77 - DNA, 21 bp, synthetic construct
  misc_feature 1..21: note = Synthetic
  source 1..21: mol_type = other DNA
cttgtaaccagaccaatttaa

SEQ ID NO: 78 - DNA, 21 bp, synthetic construct
  misc_feature 1..21: note = Synthetic
  source 1..21: mol_type = other DNA
actcagtgaacaagtaaataa

SEQ ID NO: 79 - DNA, 21 bp, synthetic construct
  misc_feature 1..21: note = Synthetic
  source 1..21: mol_type = other DNA
tagccctgtatcaagtaaatt

SEQ ID NO: 80 - DNA, 21 bp, synthetic construct
  misc_feature 1..21: note = Synthetic
  source 1..21: mol_type = other DNA
acaagattccagaacatttaa

SEQ ID NO: 81 - DNA, 21 bp, synthetic construct
  misc_feature 1..21: note = Synthetic
  source 1..21: mol_type = other DNA
caagattccagaacatttaaa

SEQ ID NO: 82 - DNA, 21 bp, synthetic construct
  misc_feature 1..21: note = Synthetic
  source 1..21: mol_type = other DNA
gagagttacccaaagaattta

SEQ ID NO: 83 - DNA, 21 bp, synthetic construct
  misc_feature 1..21: note = Synthetic
  source 1..21: mol_type = other DNA
agagttacccaaagaatttaa

SEQ ID NO: 84 - DNA, 21 bp, synthetic construct
  misc_feature 1..21: note = Synthetic
  source 1..21: mol_type = other DNA
agaagaaaggtttggaaattt

SEQ ID NO: 85 - DNA, 21 bp, synthetic construct
  misc_feature 1..21: note = Synthetic
  source 1..21: mol_type = other DNA
agcccaatctataggatttat

SEQ ID NO: 86 - DNA, 21 bp, synthetic construct
  misc_feature 1..21: note = Synthetic
  source 1..21: mol_type = other DNA
gcccaatctataggatttata

SEQ ID NO: 87 - DNA, 21 bp, synthetic construct
  misc_feature 1..21: note = Synthetic
  source 1..21: mol_type = other DNA
cccaatctataggatttatat

SEQ ID NO: 88 - DNA, 21 bp, synthetic construct
  misc_feature 1..21: note = Synthetic
  source 1..21: mol_type = other DNA
cacatactacagggcatataa

SEQ ID NO: 89 - DNA, 21 bp, synthetic construct
  misc_feature 1..21: note = Synthetic
  source 1..21: mol_type = other DNA
acatactacagggcatataaa

SEQ ID NO: 90 - DNA, 21 bp, synthetic construct
  misc_feature 1..21: note = Synthetic
  source 1..21: mol_type = other DNA
ttaaagagttgccacattata

SEQ ID NO: 91 - DNA, 21 bp, synthetic construct
  misc_feature 1..21: note = Synthetic
  source 1..21: mol_type = other DNA
gttgccacattatacataata

SEQ ID NO: 92 - DNA, 21 bp, synthetic construct
  misc_feature 1..21: note = Synthetic
  source 1..21: mol_type = other DNA
agacattgaaccagctattaa

SEQ ID NO: 93 - DNA, 21 bp, synthetic construct
  misc_feature 1..21: note = Synthetic
  source 1..21: mol_type = other DNA
gacattgaaccagctattaaa

SEQ ID NO: 94 - DNA, 21 bp, synthetic construct
  misc_feature 1..21: note = Synthetic
  source 1..21: mol_type = other DNA
agggttaaagaaaggtataaa

SEQ ID NO: 95 - DNA, 21 bp, synthetic construct
  misc_feature 1..21: note = Synthetic
  source 1..21: mol_type = other DNA
atacccgagatgaccaataaa

SEQ ID NO: 96 - DNA, 21 bp, synthetic construct
  misc_feature 1..21: note = Synthetic
  source 1..21: mol_type = other DNA
caaatccacgagaagaaataa

SEQ ID NO: 97 - DNA, 21 bp, synthetic construct
  misc_feature 1..21: note = Synthetic
  source 1..21: mol_type = other DNA
acttgtgttgatggtattata

SEQ ID NO: 98 - DNA, 21 bp, synthetic construct
  misc_feature 1..21: note = Synthetic
  source 1..21: mol_type = other DNA
cttgtgttgatggtattatat

SEQ ID NO: 99 - DNA, 21 bp, synthetic construct
  misc_feature 1..21: note = Synthetic
  source 1..21: mol_type = other DNA
attcattggaaagggtataaa

SEQ ID NO: 100 - DNA, 21 bp, synthetic construct
  misc_feature 1..21: note = Synthetic
  source 1..21: mol_type = other DNA
cagtaacaagagcccattatt

SEQ ID NO: 101 - DNA, 21 bp, synthetic construct
  misc_feature 1..21: note = Synthetic
  source 1..21: mol_type = other DNA
agtaacaagagcccattattt

SEQ ID NO: 102 - DNA, 21 bp, synthetic construct
  misc_feature 1..21: note = Synthetic
  source 1..21: mol_type = other DNA
gcccaagagatccactttatt

SEQ ID NO: 103 - DNA, 21 bp, synthetic construct
  misc_feature 1..21: note = Synthetic
  source 1..21: mol_type = other DNA
cccaagagatccactttattt

SEQ ID NO: 104 - DNA, 21 bp, synthetic construct
  misc_feature 1..21: note = Synthetic
  source 1..21: mol_type = other DNA
tttcatctcacccagaatatt

SEQ ID NO: 105 - DNA, 21 bp, synthetic construct
  misc_feature 1..21: note = Synthetic
  source 1..21: mol_type = other DNA
tgttatgagatccagttattt

SEQ ID NO: 106 - DNA, 21 bp, synthetic construct
  misc_feature 1..21: note = Synthetic
  source 1..21: mol_type = other DNA
actgaactgtgagccaattaa

SEQ ID NO: 107 - DNA, 21 bp, synthetic construct
  misc_feature 1..21: note = Synthetic
  source 1..21: mol_type = other DNA
ctgaactgtgagccaattaaa

SEQ ID NO: 108 - DNA, 21 bp, synthetic construct
  misc_feature 1..21: note = Synthetic
  source 1..21: mol_type = other DNA
agtcttaggtagttctttata

SEQ ID NO: 109 - DNA, 21 bp, synthetic construct
  misc_feature 1..21: note = Synthetic
  source 1..21: mol_type = other DNA
caaaggcagcagcacaataat

SEQ ID NO: 110 - DNA, 21 bp, synthetic construct
  misc_feature 1..21: note = Synthetic
  source 1..21: mol_type = other DNA
agactggctatttgaaattat

SEQ ID NO: 111 - DNA, 21 bp, synthetic construct
  misc_feature 1..21: note = Synthetic
  source 1..21: mol_type = other DNA
gactggctatttgaaattatt

SEQ ID NO: 112 - DNA, 21 bp, synthetic construct
  misc_feature 1..21: note = Synthetic
  source 1..21: mol_type = other DNA
caccatcaagagaactaatat

SEQ ID NO: 113 - DNA, 21 bp, synthetic construct
  misc_feature 1..21: note = Synthetic
  source 1..21: mol_type = other DNA
accatcaagagaactaatata

SEQ ID NO: 114 - DNA, 21 bp, synthetic construct
  misc_feature 1..21: note = Synthetic
  source 1..21: mol_type = other DNA
ccatcaagagaactaatataa

SEQ ID NO: 115 - DNA, 21 bp, synthetic construct
  misc_feature 1..21: note = Synthetic
  source 1..21: mol_type = other DNA
ggaaagtacatagtcaaattt

SEQ ID NO: 116 - DNA, 21 bp, synthetic construct
  misc_feature 1..21: note = Synthetic
  source 1..21: mol_type = other DNA
caatgcatactacagtataaa

SEQ ID NO: 117 - DNA, 21 bp, synthetic construct
  misc_feature 1..21: note = Synthetic
  source 1..21: mol_type = other DNA
agtccttacgtgtcaataatt

SEQ ID NO: 118 - DNA, 21 bp, synthetic construct
  misc_feature 1..21: note = Synthetic
  source 1..21: mol_type = other DNA
gtccttacgtgtcaataatta

SEQ ID NO: 119 - DNA, 21 bp, synthetic construct
  misc_feature 1..21: note = Synthetic
  source 1..21: mol_type = other DNA
gacacatacaggctgaaatta

SEQ ID NO: 120 - DNA, 21 bp, synthetic construct
  misc_feature 1..21: note = Synthetic
  source 1..21: mol_type = other DNA
acacatacaggctgaaattaa

SEQ ID NO: 121 - DNA, 21 bp, synthetic construct
  misc_feature 1..21: note = Synthetic
  source 1..21: mol_type = other DNA
cacatacaggctgaaattaaa

SEQ ID NO: 122 - DNA, 21 bp, synthetic construct
  misc_feature 1..21: note = Synthetic
  source 1..21: mol_type = other DNA
caatattgaagcacctaaata

SEQ ID NO: 123 - DNA, 21 bp, synthetic construct
  misc_feature 1..21: note = Synthetic
  source 1..21: mol_type = other DNA
atggatctaacagacattata

SEQ ID NO: 124 - DNA, 21 bp, synthetic construct
  misc_feature 1..21: note = Synthetic
  source 1..21: mol_type = other DNA
cattctccaggatagattata

SEQ ID NO: 125 - DNA, 21 bp, synthetic construct
  misc_feature 1..21: note = Synthetic
  source 1..21: mol_type = other DNA
attctccaggatagattatat

SEQ ID NO: 126 - DNA, 21 bp, synthetic construct
  misc_feature 1..21: note = Synthetic
  source 1..21: mol_type = other DNA
caagatggaaactggaaattt

SEQ ID NO: 127 - DNA, 21 bp, synthetic construct
  misc_feature 1..21: note = Synthetic
  source 1..21: mol_type = other DNA
ttctatgaggtaagcttatat

SEQ ID NO: 128 - DNA, 21 bp, synthetic construct
  misc_feature 1..21: note = Synthetic
  source 1..21: mol_type = other DNA
tctatgaggtaagcttatatt

SEQ ID NO: 129 - DNA, 21 bp, synthetic construct
  misc_feature 1..21: note = Synthetic
  source 1..21: mol_type = other DNA
acaaagatcagacagaaataa

SEQ ID NO: 130 - DNA, 21 bp, synthetic construct
  misc_feature 1..21: note = Synthetic
  source 1..21: mol_type = other DNA
caaagatcagacagaaataaa

SEQ ID NO: 131 - DNA, 21 bp, synthetic construct
  misc_feature 1..21: note = Synthetic
  source 1..21: mol_type = other DNA
ataccacagacatacaaatta

SEQ ID NO: 132 - DNA, 21 bp, synthetic construct
  misc_feature 1..21: note = Synthetic
  source 1..21: mol_type = other DNA
tgaagactgccaaccatttaa

SEQ ID NO: 133 - DNA, 21 bp, synthetic construct
  misc_feature 1..21: note = Synthetic
  source 1..21: mol_type = other DNA
gaagactgccaaccatttaaa

SEQ ID NO: 134 - DNA, 21 bp, synthetic construct
  misc_feature 1..21: note = Synthetic
  source 1..21: mol_type = other DNA
ctaattcagcaacacattata

SEQ ID NO: 135 - DNA, 21 bp, synthetic construct
  misc_feature 1..21: note = Synthetic
  source 1..21: mol_type = other DNA
gatgcaggatagttcaatata

SEQ ID NO: 136 - DNA, 21 bp, synthetic construct
  misc_feature 1..21: note = Synthetic
  source 1..21: mol_type = other DNA
atgcaggatagttcaatataa

SEQ ID NO: 137 - DNA, 21 bp, synthetic construct
  misc_feature 1..21: note = Synthetic
  source 1..21: mol_type = other DNA
tgcaggatagttcaatataat

SEQ ID NO: 138 - DNA, 21 bp, synthetic construct
  misc_feature 1..21: note = Synthetic
  source 1..21: mol_type = other DNA
gtgtctgttggctgcataaat

SEQ ID NO: 139 - DNA, 21 bp, synthetic construct
  misc_feature 1..21: note = Synthetic
  source 1..21: mol_type = other DNA
ctttgtagattctggatatta

SEQ ID NO: 140 - DNA, 21 bp, synthetic construct
  misc_feature 1..21: note = Synthetic
  source 1..21: mol_type = other DNA
gcagaagctctttagtttaat

SEQ ID NO: 141 - DNA, 21 bp, synthetic construct
  misc_feature 1..21: note = Synthetic
  source 1..21: mol_type = other DNA
cagaagctctttagtttaatt

SEQ ID NO: 142 - DNA, 21 bp, synthetic construct
  misc_feature 1..21: note = Synthetic
  source 1..21: mol_type = other DNA
catcctgttactgggtatata

SEQ ID NO: 143 - DNA, 21 bp, synthetic construct
  misc_feature 1..21: note = Synthetic
  source 1..21: mol_type = other DNA
gtatatacccagaggattata

SEQ ID NO: 144 - DNA, 21 bp, synthetic construct
  misc_feature 1..21: note = Synthetic
  source 1..21: mol_type = other DNA
tatatacccagaggattataa

SEQ ID NO: 145 - DNA, 21 bp, synthetic construct
  misc_feature 1..21: note = Synthetic
  source 1..21: mol_type = other DNA
atatacccagaggattataaa

SEQ ID NO: 146 - DNA, 21 bp, synthetic construct
  misc_feature 1..21: note = Synthetic
  source 1..21: mol_type = other DNA
tatacccagaggattataaat

SEQ ID NO: 147 - DNA, 21 bp, synthetic construct
  misc_feature 1..21: note = Synthetic
  source 1..21: mol_type = other DNA
ccctagaacttaaagtataat

SEQ ID NO: 148 - DNA, 21 bp, synthetic construct
  misc_feature 1..21: note = Synthetic
  source 1..21: mol_type = other DNA
tgacaaacctggaggtaatat

SEQ ID NO: 149 - DNA, 21 bp, synthetic construct
  misc_feature 1..21: note = Synthetic
  source 1..21: mol_type = other DNA
gacaaacctggaggtaatata

SEQ ID NO: 150 - DNA, 21 bp, synthetic construct
  misc_feature 1..21: note = Synthetic
  source 1..21: mol_type = other DNA
gtccattctcaccacttatat

SEQ ID NO: 151 - DNA, 21 bp, synthetic construct
  misc_feature 1..21: note = Synthetic
  source 1..21: mol_type = other DNA
tccattctcaccacttatatt

SEQ ID NO: 152 - DNA, 21 bp, synthetic construct
  misc_feature 1..21: note = Synthetic
  source 1..21: mol_type = other DNA
ccattctcaccacttatattt

SEQ ID NO: 153 - DNA, 21 bp, synthetic construct
  misc_feature 1..21: note = Synthetic
  source 1..21: mol_type = other DNA
cattctcaccacttatattta

SEQ ID NO: 154 - DNA, 21 bp, synthetic construct
  misc_feature 1..21: note = Synthetic
  source 1..21: mol_type = other DNA
gtagatgacatgatcttatat

SEQ ID NO: 155 - DNA, 21 bp, synthetic construct
  misc_feature 1..21: note = Synthetic
  source 1..21: mol_type = other DNA
gaatagagagcccagaaataa

SEQ ID NO: 156 - DNA, 21 bp, synthetic construct
  misc_feature 1..21: note = Synthetic
  source 1..21: mol_type = other DNA
atgcttgacatcactaataat

SEQ ID NO: 157 - DNA, 21 bp, synthetic construct
  misc_feature 1..21: note = Synthetic
  source 1..21: mol_type = other DNA
tacactgttggtgtgaattta

SEQ ID NO: 158 - DNA, 21 bp, synthetic construct
  misc_feature 1..21: note = Synthetic
  source 1..21: mol_type = other DNA
acactgttggtgtgaatttaa

SEQ ID NO: 159 - DNA, 21 bp, synthetic construct
  misc_feature 1..21: note = Synthetic
  source 1..21: mol_type = other DNA
cactgttggtgtgaatttaaa

SEQ ID NO: 160 - DNA, 21 bp, synthetic construct
  misc_feature 1..21: note = Synthetic
  source 1..21: mol_type = other DNA
ggtatttgcacactcatattt

SEQ ID NO: 161 - DNA, 21 bp, synthetic construct
  misc_feature 1..21: note = Synthetic
  source 1..21: mol_type = other DNA
gtatttgcacactcatattta

SEQ ID NO: 162 - DNA, 21 bp, synthetic construct
  misc_feature 1..21: note = Synthetic
  source 1..21: mol_type = other DNA
ataaagagaacgtggtatata

SEQ ID NO: 163 - DNA, 21 bp, synthetic construct
  misc_feature 1..21: note = Synthetic
  source 1..21: mol_type = other DNA
agacacagtggaatctattta

SEQ ID NO: 164 - DNA, 21 bp, synthetic construct
  misc_feature 1..21: note = Synthetic
  source 1..21: mol_type = other DNA
ggtacaaactttcagttataa

SEQ ID NO: 165 - DNA, 21 bp, synthetic construct
  misc_feature 1..21: note = Synthetic
  source 1..21: mol_type = other DNA
gaaggcactgatatgttaatt

SEQ ID NO: 166 - DNA, 21 bp, synthetic construct
  misc_feature 1..21: note = Synthetic
  source 1..21: mol_type = other DNA
aggcactgatatgttaattat

SEQ ID NO: 167 - DNA, 21 bp, synthetic construct
  misc_feature 1..21: note = Synthetic
  source 1..21: mol_type = other DNA
atccattcaagctagattatt

SEQ ID NO: 168 - DNA, 21 bp, synthetic construct
  misc_feature 1..21: note = Synthetic
  source 1..21: mol_type = other DNA
tccattcaagctagattattt

SEQ ID NO: 169 - DNA, 21 bp, synthetic construct
  misc_feature 1..21: note = Synthetic
  source 1..21: mol_type = other DNA
ggcatggtggccctcaattat

SEQ ID NO: 170 - DNA, 21 bp, synthetic construct
  misc_feature 1..21: note = Synthetic
  source 1..21: mol_type = other DNA
gcatggtggccctcaattata

SEQ ID NO: 171 - DNA, 21 bp, synthetic construct
  misc_feature 1..21: note = Synthetic
  source 1..21: mol_type = other DNA
catggtggccctcaattatat

SEQ ID NO: 172 - DNA, 21 bp, synthetic construct
  misc_feature 1..21: note = Synthetic
  source 1..21: mol_type = other DNA
atggtggccctcaattatata

SEQ ID NO: 173 - DNA, 21 bp, synthetic construct
  misc_feature 1..21: note = Synthetic
  source 1..21: mol_type = other DNA
tggtggccctcaattatatat

SEQ ID NO: 174 - DNA, 21 bp, synthetic construct
  misc_feature 1..21: note = Synthetic
  source 1..21: mol_type = other DNA
ggtggccctcaattatatata

SEQ ID NO: 175 - DNA, 21 bp, synthetic construct
  misc_feature 1..21: note = Synthetic
  source 1..21: mol_type = other DNA
gtggccctcaattatatatat

SEQ ID NO: 176 - DNA, 21 bp, synthetic construct
  misc_feature 1..21: note = Synthetic
  source 1..21: mol_type = other DNA
tggccctcaattatatatata

SEQ ID NO: 177 - DNA, 21 bp, synthetic construct
  misc_feature 1..21: note = Synthetic
  source 1..21: mol_type = other DNA
ggccctcaattatatatataa

SEQ ID NO: 178 - DNA, 21 bp, synthetic construct
  misc_feature 1..21: note = Synthetic
  source 1..21: mol_type = other DNA
atgaacaacaatgggatttaa

SEQ ID NO: 179 - DNA, 21 bp, synthetic construct
  misc_feature 1..21: note = Synthetic
  source 1..21: mol_type = other DNA
tgaacaacaatgggatttaaa

SEQ ID NO: 180 - DNA, 21 bp, synthetic construct
  misc_feature 1..21: note = Synthetic
  source 1..21: mol_type = other DNA
gaacaacaatgggatttaaat

SEQ ID NO: 181 - DNA, 21 bp, synthetic construct
  misc_feature 1..21: note = Synthetic
  source 1..21: mol_type = other DNA
agcaatagtggagaaatataa

SEQ ID NO: 182 - DNA, 21 bp, synthetic construct
  misc_feature 1..21: note = Synthetic
  source 1..21: mol_type = other DNA
gacctaaaccctatcttataa

SEQ ID NO: 183 - DNA, 21 bp, synthetic construct
  misc_feature 1..21: note = Synthetic
  source 1..21: mol_type = other DNA
acctaaaccctatcttataat

SEQ ID NO: 184 - DNA, 21 bp, synthetic construct
  misc_feature 1..21: note = Synthetic
  source 1..21: mol_type = other DNA
cctaaaccctatcttataatt

SEQ ID NO: 185 - DNA, 21 bp, synthetic construct
  misc_feature 1..21: note = Synthetic
  source 1..21: mol_type = other DNA
ctagagcagcaatactaatat

SEQ ID NO: 186 - DNA, 21 bp, synthetic construct
  misc_feature 1..21: note = Synthetic
  source 1..21: mol_type = other DNA
ctgaagtgcacatggaatatt

SEQ ID NO: 187 - DNA, 21 bp, synthetic construct
  misc_feature 1..21: note = Synthetic
  source 1..21: mol_type = other DNA
tcttcaggatgacacatatta

SEQ ID NO: 188 - DNA, 21 bp, synthetic construct
  misc_feature 1..21: note = Synthetic
  source 1..21: mol_type = other DNA
cacaatgatatggagatttaa

SEQ ID NO: 189 - DNA, 21 bp, synthetic construct
  misc_feature 1..21: note = Synthetic
  source 1..21: mol_type = other DNA
ctcaatccaataacctaattt

SEQ ID NO: 190 - DNA, 21 bp, synthetic construct
  misc_feature 1..21: note = Synthetic
  source 1..21: mol_type = other DNA
gtaaacagaaggaagaaataa

SEQ ID NO: 191 - DNA, 21 bp, synthetic construct
  misc_feature 1..21: note = Synthetic
  source 1..21: mol_type = other DNA
gatatcaccttacagaaatta

SEQ ID NO: 192 - DNA, 21 bp, synthetic construct
  misc_feature 1..21: note = Synthetic
  source 1..21: mol_type = other DNA
aggtccgtattacccttatat

SEQ ID NO: 193 - DNA, 21 bp, synthetic construct
  misc_feature 1..21: note = Synthetic
  source 1..21: mol_type = other DNA
catctagcctttctgatttat

SEQ ID NO: 194 - DNA, 21 bp, synthetic construct
  misc_feature 1..21: note = Synthetic
  source 1..21: mol_type = other DNA
atctagcctttctgatttata

SEQ ID NO: 195 - DNA, 21 bp, synthetic construct
  misc_feature 1..21: note = Synthetic
  source 1..21: mol_type = other DNA
tctagcctttctgatttatat

SEQ ID NO: 196 - DNA, 21 bp, synthetic construct
  misc_feature 1..21: note = Synthetic
  source 1..21: mol_type = other DNA
ctagcctttctgatttatata

SEQ ID NO: 197 - DNA, 21 bp, synthetic construct
  misc_feature 1..21: note = Synthetic
  source 1..21: mol_type = other DNA
agtcacaactcagatttattt

SEQ ID NO: 198 - DNA, 21 bp, synthetic construct
  misc_feature 1..21: note = Synthetic
  source 1..21: mol_type = other DNA
ggcctcacaactcagatttaa

SEQ ID NO: 199 - DNA, 21 bp, synthetic construct
  misc_feature 1..21: note = Synthetic
  source 1..21: mol_type = other DNA
gaacagagccctcagaaataa

SEQ ID NO: 200 - DNA, 21 bp, synthetic construct
  misc_feature 1..21: note = Synthetic
  source 1..21: mol_type = other DNA
ggaaaggattccctatttaat

SEQ ID NO: 201 - DNA, 21 bp, synthetic construct
  misc_feature 1..21: note = Synthetic
  source 1..21: mol_type = other DNA
gaaaggattccctatttaata

SEQ ID NO: 202 - DNA, 21 bp, synthetic construct
  misc_feature 1..21: note = Synthetic
  source 1..21: mol_type = other DNA
gacaaacgggatctcattaaa

SEQ ID NO: 203 - DNA, 21 bp, synthetic construct
  misc_feature 1..21: note = Synthetic
  source 1..21: mol_type = other DNA
catctgacaaagggctaatat

SEQ ID NO: 204 - DNA, 21 bp, synthetic construct
  misc_feature 1..21: note = Synthetic
  source 1..21: mol_type = other DNA
acaatgaactcagacaaattt

SEQ ID NO: 205 - DNA, 21 bp, synthetic construct
  misc_feature 1..21: note = Synthetic
  source 1..21: mol_type = other DNA
caatgaactcagacaaattta

SEQ ID NO: 206 - DNA, 21 bp, synthetic construct
  misc_feature 1..21: note = Synthetic
  source 1..21: mol_type = other DNA
gttagaatggcgatcattaaa

SEQ ID NO: 207 - DNA, 21 bp, synthetic construct
  misc_feature 1..21: note = Synthetic
  source 1..21: mol_type = other DNA
cagccatcccattgctatata

SEQ ID NO: 208 - DNA, 21 bp, synthetic construct
  misc_feature 1..21: note = Synthetic
  source 1..21: mol_type = other DNA
agccatcccattgctatatat

SEQ ID NO: 209 - DNA, 21 bp, synthetic construct
  misc_feature 1..21: note = Synthetic
  source 1..21: mol_type = other DNA
gccatcccattgctatatata

SEQ ID NO: 210 - DNA, 21 bp, synthetic construct
  misc_feature 1..21: note = Synthetic
  source 1..21: mol_type = other DNA
ccatcccattgctatatatat

SEQ ID NO: 211 - DNA, 21 bp, synthetic construct
  misc_feature 1..21: note = Synthetic
  source 1..21: mol_type = other DNA
catcccattgctatatatata

SEQ ID NO: 212 - DNA, 21 bp, synthetic construct
  misc_feature 1..21: note = Synthetic
  source 1..21: mol_type = other DNA
atatacccaaaggactataaa

SEQ ID NO: 213 - DNA, 21 bp, synthetic construct
  misc_feature 1..21: note = Synthetic
  source 1..21: mol_type = other DNA
tatacccaaaggactataaat

SEQ ID NO: 214 - DNA, 21 bp, synthetic construct
  misc_feature 1..21: note = Synthetic
  source 1..21: mol_type = other DNA
atatgcacaggtatgtttatt

SEQ ID NO: 215 - DNA, 21 bp, synthetic construct
  misc_feature 1..21: note = Synthetic
  source 1..21: mol_type = other DNA
gaaagacactgattgtattta

SEQ ID NO: 216 - DNA, 21 bp, synthetic construct
  misc_feature 1..21: note = Synthetic
  source 1..21: mol_type = other DNA
acatagccctaaccatataaa

SEQ ID NO: 217 - DNA, 21 bp, synthetic construct
  misc_feature 1..21: note = Synthetic
  source 1..21: mol_type = other DNA
cctcacacaaagacctattat

SEQ ID NO: 218 - DNA, 21 bp, synthetic construct
  misc_feature 1..21: note = Synthetic
  source 1..21: mol_type = other DNA
tggcagagatttggcaattat

SEQ ID NO: 219 - DNA, 21 bp, synthetic construct
  misc_feature 1..21: note = Synthetic
  source 1..21: mol_type = other DNA
ggcagagatttggcaattata

SEQ ID NO: 220 - DNA, 21 bp, synthetic construct
  misc_feature 1..21: note = Synthetic
  source 1..21: mol_type = other DNA
agaacaacaaggaggaaattt

SEQ ID NO: 221 - DNA, 21 bp, synthetic construct
  misc_feature 1..21: note = Synthetic
  source 1..21: mol_type = other DNA
gaacaacaaggaggaaattta

SEQ ID NO: 222 - DNA, 21 bp, synthetic construct
  misc_feature 1..21: note = Synthetic
  source 1..21: mol_type = other DNA
gaatggagtgaaatgtttaaa

SEQ ID NO: 223 - DNA, 21 bp, synthetic construct
  misc_feature 1..21: note = Synthetic
  source 1..21: mol_type = other DNA
tctgatttccttgggtttatt

SEQ ID NO: 224 - DNA, 21 bp, synthetic construct
  misc_feature 1..21: note = Synthetic
  source 1..21: mol_type = other DNA
ctgatttccttgggtttattt

SEQ ID NO: 225 - DNA, 21 bp, synthetic construct
  misc_feature 1..21: note = Synthetic
  source 1..21: mol_type = other DNA
tgctcttctttctactttatt

SEQ ID NO: 226 - DNA, 21 bp, synthetic construct
  misc_feature 1..21: note = Synthetic
  source 1..21: mol_type = other DNA
gtaagcatttagtgctataaa

SEQ ID NO: 227 - DNA, 21 bp, synthetic construct
  misc_feature 1..21: note = Synthetic
  source 1..21: mol_type = other DNA
ttagtcacatcccacaaattt

SEQ ID NO: 228 - DNA, 21 bp, synthetic construct
  misc_feature 1..21: note = Synthetic
  source 1..21: mol_type = other DNA
atggtctgtgctgtgaatatt

SEQ ID NO: 229 - DNA, 21 bp, synthetic construct
  misc_feature 1..21: note = Synthetic
  source 1..21: mol_type = other DNA
ttgaagtctccaaccataatt

SEQ ID NO: 230 - DNA, 21 bp, synthetic construct
  misc_feature 1..21: note = Synthetic
  source 1..21: mol_type = other DNA
cagtttgtgcatcacatattt

SEQ ID NO: 231 - DNA, 21 bp, synthetic construct
  misc_feature 1..21: note = Synthetic
  source 1..21: mol_type = other DNA
tgccctcttggtggcttattt

SEQ ID NO: 232 - DNA, 21 bp, synthetic construct
  misc_feature 1..21: note = Synthetic
  source 1..21: mol_type = other DNA
tcctaggtcataatgataatt

SEQ ID NO: 233 - DNA, 21 bp, synthetic construct
  misc_feature 1..21: note = Synthetic
  source 1..21: mol_type = other DNA
ctccacatccttaccaatatt

SEQ ID NO: 234 - DNA, 21 bp, synthetic construct
  misc_feature 1..21: note = Synthetic
  source 1..21: mol_type = other DNA
cgcttatcagatatgatttat

SEQ ID NO: 235 - DNA, 21 bp, synthetic construct
  misc_feature 1..21: note = Synthetic
  source 1..21: mol_type = other DNA
ggtctatacatgtagattatt

SEQ ID NO: 236 - DNA, 21 bp, synthetic construct
  misc_feature 1..21: note = Synthetic
  source 1..21: mol_type = other DNA
tcatagatgtatgggattatt

SEQ ID NO: 237 - DNA, 21 bp, synthetic construct
  misc_feature 1..21: note = Synthetic
  source 1..21: mol_type = other DNA
catagatgtatgggattattt

SEQ ID NO: 238 - DNA, 21 bp, synthetic construct
  misc_feature 1..21: note = Synthetic
  source 1..21: mol_type = other DNA
cttgtaactccttggttaaat

SEQ ID NO: 239 - DNA, 21 bp, synthetic construct
  misc_feature 1..21: note = Synthetic
  source 1..21: mol_type = other DNA
ttgtaactccttggttaaatt

SEQ ID NO: 240 - DNA, 21 bp, synthetic construct
  misc_feature 1..21: note = Synthetic
  source 1..21: mol_type = other DNA
tgtaactccttggttaaattt

SEQ ID NO: 241 - DNA, 21 bp, synthetic construct
  misc_feature 1..21: note = Synthetic
  source 1..21: mol_type = other DNA
gtaactccttggttaaattta

SEQ ID NO: 242 - DNA, 21 bp, synthetic construct
  misc_feature 1..21: note = Synthetic
  source 1..21: mol_type = other DNA
tcctttctgatgctgtttaaa

SEQ ID NO: 243 - DNA, 21 bp, synthetic construct
  misc_feature 1..21: note = Synthetic
  source 1..21: mol_type = other DNA
cctttctgatgctgtttaaat

SEQ ID NO: 244 - DNA, 21 bp, synthetic construct
  misc_feature 1..21: note = Synthetic
  source 1..21: mol_type = other DNA
ctgaaactttgctgcatttat

SEQ ID NO: 245 - DNA, 21 bp, synthetic construct
  misc_feature 1..21: note = Synthetic
  source 1..21: mol_type = other DNA
tgaaactttgctgcatttatt

SEQ ID NO: 246 - DNA, 21 bp, synthetic construct
  misc_feature 1..21: note = Synthetic
  source 1..21: mol_type = other DNA
gaaactttgctgcatttatta

SEQ ID NO: 247 - DNA, 21 bp, synthetic construct
  misc_feature 1..21: note = Synthetic
  source 1..21: mol_type = other DNA
ttgctctgtgaatagataatt

SEQ ID NO: 248 - DNA, 21 bp, synthetic construct
  misc_feature 1..21: note = Synthetic
  source 1..21: mol_type = other DNA
tgctctgtgaatagataattt

SEQ ID NO: 249 - DNA, 21 bp, synthetic construct
  misc_feature 1..21: note = Synthetic
  source 1..21: mol_type = other DNA
atatgttgatccacctttata

SEQ ID NO: 250 - DNA, 21 bp, synthetic construct
  misc_feature 1..21: note = Synthetic
  source 1..21: mol_type = other DNA
tatgttgatccacctttatat

SEQ ID NO: 251 - DNA, 21 bp, synthetic construct
  misc_feature 1..21: note = Synthetic
  source 1..21: mol_type = other DNA
atgttgatccacctttatatt

SEQ ID NO: 252 - DNA, 21 bp, synthetic construct
  misc_feature 1..21: note = Synthetic
  source 1..21: mol_type = other DNA
tctttggctactttgtataat

SEQ ID NO: 253 - DNA, 21 bp, synthetic construct
  misc_feature 1..21: note = Synthetic
  source 1..21: mol_type = other DNA
ctttggctactttgtataata

SEQ ID NO: 254 - DNA, 21 bp, synthetic construct
  misc_feature 1..21: note = Synthetic
  source 1..21: mol_type = other DNA
taagtgcatagagccaataaa

SEQ ID NO: 255 - DNA, 21 bp, synthetic construct
  misc_feature 1..21: note = Synthetic
  source 1..21: mol_type = other DNA
atgcacatgatcttcttaatt

SEQ ID NO: 256 - DNA, 21 bp, synthetic construct
  misc_feature 1..21: note = Synthetic
  source 1..21: mol_type = other DNA
tgcacatgatcttcttaattt

SEQ ID NO: 257 - DNA, 21 bp, synthetic construct
  misc_feature 1..21: note = Synthetic
  source 1..21: mol_type = other DNA
tggcttaaacaagagaaattt

SEQ ID NO: 258 - DNA, 21 bp, synthetic construct
  misc_feature 1..21: note = Synthetic
  source 1..21: mol_type = other DNA
ggcttaaacaagagaaattta

SEQ ID NO: 259 - DNA, 21 bp, synthetic construct
  misc_feature 1..21: note = Synthetic
  source 1..21: mol_type = other DNA
atatgttgatccacctttata

SEQ ID NO: 260 - DNA, 21 bp, synthetic construct
  misc_feature 1..21: note = Synthetic
  source 1..21: mol_type = other DNA
tatgttgatccacctttatat

SEQ ID NO: 261 - DNA, 21 bp, synthetic construct
  misc_feature 1..21: note = Synthetic
  source 1..21: mol_type = other DNA
atgttgatccacctttatatt

SEQ ID NO: 262 - DNA, 21 bp, synthetic construct
  misc_feature 1..21: note = Synthetic
  source 1..21: mol_type = other DNA
tctttggctactttgtataat

SEQ ID NO: 263 - DNA, 21 bp, synthetic construct
  misc_feature 1..21: note = Synthetic
  source 1..21: mol_type = other DNA
ctttggctactttgtataata

SEQ ID NO: 264 - DNA, 21 bp, synthetic construct
  misc_feature 1..21: note = Synthetic
  source 1..21: mol_type = other DNA
taagtgcatagagccaataaa

SEQ ID NO: 265 - DNA, 21 bp, synthetic construct
  misc_feature 1..21: note = Synthetic
  source 1..21: mol_type = other DNA
atgcacatgatcttcttaatt

SEQ ID NO: 266 - DNA, 21 bp, synthetic construct
  misc_feature 1..21: note = Synthetic
  source 1..21: mol_type = other DNA
tgcacatgatcttcttaattt

SEQ ID NO: 267 - DNA, 21 bp, synthetic construct
  misc_feature 1..21: note = Synthetic
  source 1..21: mol_type = other DNA
tggcttaaacaagagaaattt

SEQ ID NO: 268 - DNA, 21 bp, synthetic construct
  misc_feature 1..21: note = Synthetic
  source 1..21: mol_type = other DNA
ggcttaaacaagagaaattta

SEQ ID NO: 269 - DNA, 21 bp, synthetic construct
  misc_feature 1..21: note = Synthetic
  source 1..21: mol_type = other DNA
aggtatcagagtagtatattt

SEQ ID NO: 270 - DNA, 21 bp, synthetic construct
  misc_feature 1..21: note = Synthetic
  source 1..21: mol_type = other DNA
agttcatcgttagtgttatat

SEQ ID NO: 271 - DNA, 21 bp, synthetic construct
  misc_feature 1..21: note = Synthetic
  source 1..21: mol_type = other DNA
gttcatcgttagtgttatata

SEQ ID NO: 272 - DNA, 21 bp, synthetic construct
  misc_feature 1..21: note = Synthetic
  source 1..21: mol_type = other DNA
accaccatgcccagctaattt

SEQ ID NO: 273 - DNA, 21 bp, synthetic construct
  misc_feature 1..21: note = Synthetic
  source 1..21: mol_type = other DNA
atgacccatttgaagttaatt

SEQ ID NO: 274 - DNA, 21 bp, synthetic construct
  misc_feature 1..21: note = Synthetic
  source 1..21: mol_type = other DNA
tgacccatttgaagttaattt

SEQ ID NO: 275 - DNA, 21 bp, synthetic construct
  misc_feature 1..21: note = Synthetic
  source 1..21: mol_type = other DNA
atgctggcctcactgaataaa

SEQ ID NO: 276 - DNA, 21 bp, synthetic construct
  misc_feature 1..21: note = Synthetic
  source 1..21: mol_type = other DNA
tgctggcctcactgaataaat

SEQ ID NO: 277 - DNA, 21 bp, synthetic construct
  misc_feature 1..21: note = Synthetic
  source 1..21: mol_type = other DNA
gctggcctcactgaataaatt

SEQ ID NO: 278 - DNA, 21 bp, synthetic construct
  misc_feature 1..21: note = Synthetic
  source 1..21: mol_type = other DNA
tgttccctcctcttcaattat

SEQ ID NO: 279 - DNA, 21 bp, synthetic construct
  misc_feature 1..21: note = Synthetic
  source 1..21: mol_type = other DNA
gttccctcctcttcaattatt

SEQ ID NO: 280 - DNA, 21 bp, synthetic construct
  misc_feature 1..21: note = Synthetic
  source 1..21: mol_type = other DNA
ttccctcctcttcaattattt

SEQ ID NO: 281 - DNA, 21 bp, synthetic construct
  misc_feature 1..21: note = Synthetic
  source 1..21: mol_type = other DNA
ttggtaggttgtgcgtattta

SEQ ID NO: 282 - DNA, 21 bp, synthetic construct
  misc_feature 1..21: note = Synthetic
  source 1..21: mol_type = other DNA
attagttggcatgcaattatt

SEQ ID NO: 283 - DNA, 21 bp, synthetic construct
  misc_feature 1..21: note = Synthetic
  source 1..21: mol_type = other DNA
attcgtagttctctgaataat

SEQ ID NO: 284 - DNA, 21 bp, synthetic construct
  misc_feature 1..21: note = Synthetic
  source 1..21: mol_type = other DNA
ttccttctgttggcctttaat

SEQ ID NO: 285 - DNA, 21 bp, synthetic construct
  misc_feature 1..21: note = Synthetic
  source 1..21: mol_type = other DNA
tccttctgttggcctttaatt

SEQ ID NO: 286 - DNA, 21 bp, synthetic construct
  misc_feature 1..21: note = Synthetic
  source 1..21: mol_type = other DNA
ccttctgttggcctttaattt

SEQ ID NO: 287 - DNA, 21 bp, synthetic construct
  misc_feature 1..21: note = Synthetic
  source 1..21: mol_type = other DNA
gaaagcatttagagctataaa

SEQ ID NO: 288 - DNA, 21 bp, synthetic construct
  misc_feature 1..21: note = Synthetic
  source 1..21: mol_type = other DNA
ctttcactacctgccataaat

SEQ ID NO: 289 - DNA, 21 bp, synthetic construct
  misc_feature 1..21: note = Synthetic
  source 1..21: mol_type = other DNA
tttcactacctgccataaatt

SEQ ID NO: 290 - DNA, 21 bp, synthetic construct
  misc_feature 1..21: note = Synthetic
  source 1..21: mol_type = other DNA
ttcactacctgccataaattt

SEQ ID NO: 291 - DNA, 21 bp, synthetic construct
  misc_feature 1..21: note = Synthetic
  source 1..21: mol_type = other DNA
tccttgacctattggttattt

SEQ ID NO: 292 - DNA, 21 bp, synthetic construct
  misc_feature 1..21: note = Synthetic
  source 1..21: mol_type = other DNA
ccttgacctattggttattta

SEQ ID NO: 293 - DNA, 21 bp, synthetic construct
  misc_feature 1..21: note = Synthetic
  source 1..21: mol_type = other DNA
cttgacctattggttatttaa

SEQ ID NO: 294 - DNA, 21 bp, synthetic construct
  misc_feature 1..21: note = Synthetic
  source 1..21: mol_type = other DNA
ttgtgatcacagaagatattt

SEQ ID NO: 295 - DNA, 21 bp, synthetic construct
  misc_feature 1..21: note = Synthetic
  source 1..21: mol_type = other DNA
tgtataatcgcagtctattaa

SEQ ID NO: 296 - DNA, 21 bp, synthetic construct
  misc_feature 1..21: note = Synthetic
  source 1..21: mol_type = other DNA
tcgcagtctattaacatttat

SEQ ID NO: 297 - DNA, 21 bp, synthetic construct
  misc_feature 1..21: note = Synthetic
  source 1..21: mol_type = other DNA
cgcagtctattaacatttatt

SEQ ID NO: 298 - DNA, 21 bp, synthetic construct
  misc_feature 1..21: note = Synthetic
  source 1..21: mol_type = other DNA
gagtggtaaagtctctattat

SEQ ID NO: 299 - DNA, 21 bp, synthetic construct
  misc_feature 1..21: note = Synthetic
  source 1..21: mol_type = other DNA
agtggtaaagtctctattatt

SEQ ID NO: 300 - DNA, 21 bp, synthetic construct
  misc_feature 1..21: note = Synthetic
  source 1..21: mol_type = other DNA
agcataagctatgtcattaaa

SEQ ID NO: 301 - DNA, 21 bp, synthetic construct
  misc_feature 1..21: note = Synthetic
  source 1..21: mol_type = other DNA
ctcttcatttccttcaatatt

SEQ ID NO: 302 - DNA, 21 bp, synthetic construct
  misc_feature 1..21: note = Synthetic
  source 1..21: mol_type = other DNA
tgagatacctagaacaatata

SEQ ID NO: 303 - DNA, 21 bp, synthetic construct
  misc_feature 1..21: note = Synthetic
  source 1..21: mol_type = other DNA
gagatacctagaacaatataa

SEQ ID NO: 304 - DNA, 21 bp, synthetic construct
  misc_feature 1..21: note = Synthetic
  source 1..21: mol_type = other DNA
ctctttctctgtgagattata

SEQ ID NO: 305 - DNA, 21 bp, synthetic construct
  misc_feature 1..21: note = Synthetic
  source 1..21: mol_type = other DNA
acaacagcctggaagtataat

SEQ ID NO: 306 - DNA, 21 bp, synthetic construct
  misc_feature 1..21: note = Synthetic
  source 1..21: mol_type = other DNA
caacagcctggaagtataatt

SEQ ID NO: 307 - DNA, 21 bp, synthetic construct
  misc_feature 1..21: note = Synthetic
  source 1..21: mol_type = other DNA
acagcctggaagtataattaa

SEQ ID NO: 308 - DNA, 21 bp, synthetic construct
  misc_feature 1..21: note = Synthetic
  source 1..21: mol_type = other DNA
attcaaactgatgccaattta

SEQ ID NO: 309 - DNA, 21 bp, synthetic construct
  misc_feature 1..21: note = Synthetic
  source 1..21: mol_type = other DNA
ttcaaactgatgccaatttaa

SEQ ID NO: 310 - DNA, 21 bp, synthetic construct
  misc_feature 1..21: note = Synthetic
  source 1..21: mol_type = other DNA
agtcaacacaccaatattaaa

SEQ ID NO: 311 - DNA, 21 bp, synthetic construct
  misc_feature 1..21: note = Synthetic
  source 1..21: mol_type = other DNA
agctcctgtttgaagtaaatt

SEQ ID NO: 312 - DNA, 21 bp, synthetic construct
  misc_feature 1..21: note = Synthetic
  source 1..21: mol_type = other DNA
gctcctgtttgaagtaaattt

SEQ ID NO: 313 - DNA, 21 bp, synthetic construct
  misc_feature 1..21: note = Synthetic
  source 1..21: mol_type = other DNA
gccttccaaggtttctattaa

SEQ ID NO: 314 - DNA, 21 bp, synthetic construct
  misc_feature 1..21: note = Synthetic
  source 1..21: mol_type = other DNA
tgtgggtctctttggatttat

SEQ ID NO: 315 - DNA, 21 bp, synthetic construct
  misc_feature 1..21: note = Synthetic
  source 1..21: mol_type = other DNA
tatggttctgtagagatattt

SEQ ID NO: 316 - DNA, 21 bp, synthetic construct
  misc_feature 1..21: note = Synthetic
  source 1..21: mol_type = other DNA
tgttctcaatttccctatata

SEQ ID NO: 317 - DNA, 21 bp, synthetic construct
  misc_feature 1..21: note = Synthetic
  source 1..21: mol_type = other DNA
gttctcaatttccctatataa

SEQ ID NO: 318 - DNA, 21 bp, synthetic construct
  misc_feature 1..21: note = Synthetic
  source 1..21: mol_type = other DNA
aggttggaacatttcaaataa

SEQ ID NO: 319 - DNA, 21 bp, synthetic construct
  misc_feature 1..21: note = Synthetic
  source 1..21: mol_type = other DNA
ttacatgggctgttctataaa

SEQ ID NO: 320 - DNA, 21 bp, synthetic construct
  misc_feature 1..21: note = Synthetic
  source 1..21: mol_type = other DNA
tacatgggctgttctataaat

SEQ ID NO: 321 - DNA, 21 bp, synthetic construct
  misc_feature 1..21: note = Synthetic
  source 1..21: mol_type = other DNA
tgttacttaaggtggttaata

SEQ ID NO: 322 - DNA, 21 bp, synthetic construct
  misc_feature 1..21: note = Synthetic
  source 1..21: mol_type = other DNA
gttacttaaggtggttaataa

SEQ ID NO: 323 - DNA, 21 bp, synthetic construct
  misc_feature 1..21: note = Synthetic
  source 1..21: mol_type = other DNA
gttgctcaagtcttctatatt

SEQ ID NO: 324 - DNA, 21 bp, synthetic construct
  misc_feature 1..21: note = Synthetic
  source 1..21: mol_type = other DNA
caacatgcaggtttgttatat

SEQ ID NO: 325 - DNA, 21 bp, synthetic construct
  misc_feature 1..21: note = Synthetic
  source 1..21: mol_type = other DNA
acatgcaggtttgttatatat

SEQ ID NO: 326 - DNA, 21 bp, synthetic construct
  misc_feature 1..21: note = Synthetic
  source 1..21: mol_type = other DNA
acgtgtgcatgtgtctttata

SEQ ID NO: 327 - DNA, 21 bp, synthetic construct
  misc_feature 1..21: note = Synthetic
  source 1..21: mol_type = other DNA
ctttatagcagcatgatttat

SEQ ID NO: 328 - DNA, 21 bp, synthetic construct
  misc_feature 1..21: note = Synthetic
  source 1..21: mol_type = other DNA
tgtgtctttggctgcataaat

SEQ ID NO: 329 - DNA, 21 bp, synthetic construct
  misc_feature 1..21: note = Synthetic
  source 1..21: mol_type = other DNA
ctttgtagattctggatatta

SEQ ID NO: 330 - DNA, 21 bp, synthetic construct
  misc_feature 1..21: note = Synthetic
  source 1..21: mol_type = other DNA
gcagaagctctttagtttaat

SEQ ID NO: 331 - DNA, 21 bp, synthetic construct
  misc_feature 1..21: note = Synthetic
  source 1..21: mol_type = other DNA
tttcccagcaccatttattaa

SEQ ID NO: 332 - DNA, 21 bp, synthetic construct
  misc_feature 1..21: note = Synthetic
  source 1..21: mol_type = other DNA
ttcccagcaccatttattaaa

SEQ ID NO: 333 - DNA, 21 bp, synthetic construct
  misc_feature 1..21: note = Synthetic
  source 1..21: mol_type = other DNA
tcccagcaccatttattaaat

SEQ ID NO: 334 - DNA, 21 bp, synthetic construct
  misc_feature 1..21: note = Synthetic
  source 1..21: mol_type = other DNA
cccagcaccatttattaaata

SEQ ID NO: 335 - DNA, 21 bp, synthetic construct
  misc_feature 1..21: note = Synthetic
  source 1..21: mol_type = other DNA
gttgtagatgtgtggtattat

SEQ ID NO: 336 - DNA, 21 bp, synthetic construct
  misc_feature 1..21: note = Synthetic
  source 1..21: mol_type = other DNA
ttgtagatgtgtggtattatt

SEQ ID NO: 337 - DNA, 21 bp, synthetic construct
  misc_feature 1..21: note = Synthetic
  source 1..21: mol_type = other DNA
tgtagatgtgtggtattattt

SEQ ID NO: 338 - DNA, 21 bp, synthetic construct
  misc_feature 1..21: note = Synthetic
  source 1..21: mol_type = other DNA
gttctgttccattggtttata

SEQ ID NO: 339 - DNA, 21 bp, synthetic construct
  misc_feature 1..21: note = Synthetic
  source 1..21: mol_type = other DNA
ttctgttccattggtttatat

SEQ ID NO: 340 - DNA, 21 bp, synthetic construct
  misc_feature 1..21: note = Synthetic
  source 1..21: mol_type = other DNA
ggatggcattgaatctataaa

SEQ ID NO: 341 - DNA, 21 bp, synthetic construct
  misc_feature 1..21: note = Synthetic
  source 1..21: mol_type = other DNA
gatggcattgaatctataaat

SEQ ID NO: 342 - DNA, 21 bp, synthetic construct
  misc_feature 1..21: note = Synthetic
  source 1..21: mol_type = other DNA
cctaattgaataccctttatt

SEQ ID NO: 343 - DNA, 21 bp, synthetic construct
  misc_feature 1..21: note = Synthetic
  source 1..21: mol_type = other DNA
ggctgtgggtttgtcataaat

SEQ ID NO: 344 - DNA, 21 bp, synthetic construct
  misc_feature 1..21: note = Synthetic
  source 1..21: mol_type = other DNA
gctgtgggtttgtcataaata

SEQ ID NO: 345 - DNA, 21 bp, synthetic construct
  misc_feature 1..21: note = Synthetic
  source 1..21: mol_type = other DNA
tgtcccatcaatacctaattt

SEQ ID NO: 346 - DNA, 21 bp, synthetic construct
  misc_feature 1..21: note = Synthetic
  source 1..21: mol_type = other DNA
gtcccatcaatacctaattta

SEQ ID NO: 347 - DNA, 21 bp, synthetic construct
  misc_feature 1..21: note = Synthetic
  source 1..21: mol_type = other DNA
tcccatcaatacctaatttat

SEQ ID NO: 348 - DNA, 21 bp, synthetic construct
  misc_feature 1..21: note = Synthetic
  source 1..21: mol_type = other DNA
cccatcaatacctaatttatt

SEQ ID NO: 349 - DNA, 21 bp, synthetic construct
  misc_feature 1..21: note = Synthetic
  source 1..21: mol_type = other DNA
ttgtctttggttctgtttata

SEQ ID NO: 350 - DNA, 21 bp, synthetic construct
  misc_feature 1..21: note = Synthetic
  source 1..21: mol_type = other DNA
tgtctttggttctgtttatat

SEQ ID NO: 351 - DNA, 21 bp, synthetic construct
  misc_feature 1..21: note = Synthetic
  source 1..21: mol_type = other DNA
agcatgctttgctggtattaa

SEQ ID NO: 352 - DNA, 21 bp, synthetic construct
  misc_feature 1..21: note = Synthetic
  source 1..21: mol_type = other DNA
gcatgctttgctggtattaat

SEQ ID NO: 353 - DNA, 21 bp, synthetic construct
  misc_feature 1..21: note = Synthetic
  source 1..21: mol_type = other DNA
catgctttgctggtattaata

SEQ ID NO: 354 - DNA, 21 bp, synthetic construct
  misc_feature 1..21: note = Synthetic
  source 1..21: mol_type = other DNA
atgctttgctggtattaatat

SEQ ID NO: 355 - DNA, 21 bp, synthetic construct
  misc_feature 1..21: note = Synthetic
  source 1..21: mol_type = other DNA
tgctttgctggtattaatata

SEQ ID NO: 356 - DNA, 21 bp, synthetic construct
  misc_feature 1..21: note = Synthetic
  source 1..21: mol_type = other DNA
gagagttagaacctcaatata

SEQ ID NO: 357 - DNA, 21 bp, synthetic construct
  misc_feature 1..21: note = Synthetic
  source 1..21: mol_type = other DNA
agagttagaacctcaatatat

SEQ ID NO: 358 - DNA, 21 bp, synthetic construct
  misc_feature 1..21: note = Synthetic
  source 1..21: mol_type = other DNA
ccaccacgcctggctttataa

SEQ ID NO: 359 - DNA, 21 bp, synthetic construct
  misc_feature 1..21: note = Synthetic
  source 1..21: mol_type = other DNA
caccacgcctggctttataat

SEQ ID NO: 360 - DNA, 21 bp, synthetic construct
  misc_feature 1..21: note = Synthetic
  source 1..21: mol_type = other DNA
accacgcctggctttataatt

SEQ ID NO: 361 - DNA, 21 bp, synthetic construct
  misc_feature 1..21: note = Synthetic
  source 1..21: mol_type = other DNA
ccacgcctggctttataattt

SEQ ID NO: 362 - DNA, 21 bp, synthetic construct
  misc_feature 1..21: note = Synthetic
  source 1..21: mol_type = other DNA
gtctagctccaagtgatatat

SEQ ID NO: 363 - DNA, 21 bp, synthetic construct
  misc_feature 1..21: note = Synthetic
  source 1..21: mol_type = other DNA
tttgcttctgtctgaaatata

SEQ ID NO: 364 - DNA, 21 bp, synthetic construct
  misc_feature 1..21: note = Synthetic
  source 1..21: mol_type = other DNA
ttgcttctgtctgaaatatat

SEQ ID NO: 365 - DNA, 21 bp, synthetic construct
  misc_feature 1..21: note = Synthetic
  source 1..21: mol_type = other DNA
tcttaagtctgtgagtttata

SEQ ID NO: 366 - DNA, 21 bp, synthetic construct
  misc_feature 1..21: note = Synthetic
  source 1..21: mol_type = other DNA
tctctgatttccacctatatt

SEQ ID NO: 367 - DNA, 21 bp, synthetic construct
  misc_feature 1..21: note = Synthetic
  source 1..21: mol_type = other DNA
ctctgatttccacctatatta

SEQ ID NO: 368 - DNA, 21 bp, synthetic construct
  misc_feature 1..21: note = Synthetic
  source 1..21: mol_type = other DNA
gtatatactccagagaaataa

SEQ ID NO: 369 - DNA, 21 bp, synthetic construct
  misc_feature 1..21: note = Synthetic
  source 1..21: mol_type = other DNA
caccagaacttgaacattaat

SEQ ID NO: 370 - DNA, 21 bp, synthetic construct
  misc_feature 1..21: note = Synthetic
  source 1..21: mol_type = other DNA
accagaacttgaacattaatt

SEQ ID NO: 371 - DNA, 21 bp, synthetic construct
  misc_feature 1..21: note = Synthetic
  source 1..21: mol_type = other DNA
ccagaacttgaacattaattt

SEQ ID NO: 372 - DNA, 21 bp, synthetic construct
  misc_feature 1..21: note = Synthetic
  source 1..21: mol_type = other DNA
gtctggaactcctggaattaa

SEQ ID NO: 373 - DNA, 21 bp, synthetic construct
  misc_feature 1..21: note = Synthetic
  source 1..21: mol_type = other DNA
ctttctgtgcctggcttattt

SEQ ID NO: 374 - DNA, 21 bp, synthetic construct
  misc_feature 1..21: note = Synthetic
  source 1..21: mol_type = other DNA
caggatttaccttcctttaaa

SEQ ID NO: 375 - DNA, 21 bp, synthetic construct
  misc_feature 1..21: note = Synthetic
  source 1..21: mol_type = other DNA
tctgttgatgggcacttaaat

SEQ ID NO: 376 - DNA, 21 bp, synthetic construct
  misc_feature 1..21: note = Synthetic
  source 1..21: mol_type = other DNA
ctgttgatgggcacttaaatt

SEQ ID NO: 377 - DNA, 21 bp, synthetic construct
  misc_feature 1..21: note = Synthetic
  source 1..21: mol_type = other DNA
tcatagcggctgtactaattt

SEQ ID NO: 378 - DNA, 21 bp, synthetic construct
  misc_feature 1..21: note = Synthetic
  source 1..21: mol_type = other DNA
catagcggctgtactaattta

SEQ ID NO: 379 - DNA, 21 bp, synthetic construct
  misc_feature 1..21: note = Synthetic
  source 1..21: mol_type = other DNA
tttacccatttccagatatat

SEQ ID NO: 380 - DNA, 21 bp, synthetic construct
  misc_feature 1..21: note = Synthetic
  source 1..21: mol_type = other DNA
gtgtaaataagggtctaattt

SEQ ID NO: 381 - DNA, 21 bp, synthetic construct
  misc_feature 1..21: note = Synthetic
  source 1..21: mol_type = other DNA
tggttatgtcatcagtaatta

SEQ ID NO: 382 - DNA, 21 bp, synthetic construct
  misc_feature 1..21: note = Synthetic
  source 1..21: mol_type = other DNA
ccttgcatcctaaggataaat

SEQ ID NO: 383 - DNA, 21 bp, synthetic construct
  misc_feature 1..21: note = Synthetic
  source 1..21: mol_type = other DNA
gtgaatccagtttgctaatat

SEQ ID NO: 384 - DNA, 21 bp, synthetic construct
  misc_feature 1..21: note = Synthetic
  source 1..21: mol_type = other DNA
tgaatccagtttgctaatata

SEQ ID NO: 385 - DNA, 21 bp, synthetic construct
  misc_feature 1..21: note = Synthetic
  source 1..21: mol_type = other DNA
gaatccagtttgctaatatat

SEQ ID NO: 386 - DNA, 21 bp, synthetic construct
  misc_feature 1..21: note = Synthetic
  source 1..21: mol_type = other DNA
ccatgttcatcagggatatta

SEQ ID NO: 387 - DNA, 21 bp, synthetic construct
  misc_feature 1..21: note = Synthetic
  source 1..21: mol_type = other DNA
gtgtctttctctggctttaat

SEQ ID NO: 388 - DNA, 21 bp, synthetic construct
  misc_feature 1..21: note = Synthetic
  source 1..21: mol_type = other DNA
tgtctttctctggctttaata

SEQ ID NO: 389 - DNA, 21 bp, synthetic construct
  misc_feature 1..21: note = Synthetic
  source 1..21: mol_type = other DNA
gtctttctctggctttaataa

SEQ ID NO: 390 - DNA, 21 bp, synthetic construct
  misc_feature 1..21: note = Synthetic
  source 1..21: mol_type = other DNA
ttgtgatccttcttctttatt

SEQ ID NO: 391 - DNA, 21 bp, synthetic construct
  misc_feature 1..21: note = Synthetic
  source 1..21: mol_type = other DNA
gtaggtttgtatcgctataaa

SEQ ID NO: 392 - DNA, 21 bp, synthetic construct
  misc_feature 1..21: note = Synthetic
  source 1..21: mol_type = other DNA
taggtttgtatcgctataaat

SEQ ID NO: 393 - DNA, 21 bp, synthetic construct
  misc_feature 1..21: note = Synthetic
  source 1..21: mol_type = other DNA
aggtttgtatcgctataaatt

SEQ ID NO: 394 - DNA, 21 bp, synthetic construct
  misc_feature 1..21: note = Synthetic
  source 1..21: mol_type = other DNA
ggtttgtatcgctataaattt

SEQ ID NO: 395 - DNA, 21 bp, synthetic construct
  misc_feature 1..21: note = Synthetic
  source 1..21: mol_type = other DNA
tcatttgtctcaaggtaattt

SEQ ID NO: 396 - DNA, 21 bp, synthetic construct
  misc_feature 1..21: note = Synthetic
  source 1..21: mol_type = other DNA
tttgggagcatattgtttaat

SEQ ID NO: 397 - DNA, 21 bp, synthetic construct
  misc_feature 1..21: note = Synthetic
  source 1..21: mol_type = other DNA
ttgggagcatattgtttaatt

SEQ ID NO: 398 - DNA, 21 bp, synthetic construct
  misc_feature 1..21: note = Synthetic
  source 1..21: mol_type = other DNA
tgggagcatattgtttaattt

SEQ ID NO: 399 - DNA, 21 bp, synthetic construct
  misc_feature 1..21: note = Synthetic
  source 1..21: mol_type = other DNA
tggatggaatgtttcatatat

SEQ ID NO: 400 - DNA, 21 bp, synthetic construct
  misc_feature 1..21: note = Synthetic
  source 1..21: mol_type = other DNA
agtattgaggtcccgtattat

SEQ ID NO: 401 - DNA, 21 bp, synthetic construct
  misc_feature 1..21: note = Synthetic
  source 1..21: mol_type = other DNA
gtattgaggtcccgtattata

SEQ ID NO: 402 - DNA, 21 bp, synthetic construct
  misc_feature 1..21: note = Synthetic
  source 1..21: mol_type = other DNA
ctccctcttcacatcatttaa

SEQ ID NO: 403 - DNA, 21 bp, synthetic construct
  misc_feature 1..21: note = Synthetic
  source 1..21: mol_type = other DNA
tccctcttcacatcatttaaa

SEQ ID NO: 404 - DNA, 21 bp, synthetic construct
  misc_feature 1..21: note = Synthetic
  source 1..21: mol_type = other DNA
tatgtcttcttggtgaattaa

SEQ ID NO: 405 - DNA, 21 bp, synthetic construct
  misc_feature 1..21: note = Synthetic
  source 1..21: mol_type = other DNA
atgtcttcttggtgaattaat

SEQ ID NO: 406 - DNA, 21 bp, synthetic construct
  misc_feature 1..21: note = Synthetic
  source 1..21: mol_type = other DNA
ctgctctcaatttccatttat

SEQ ID NO: 407 - DNA, 21 bp, synthetic construct
  misc_feature 1..21: note = Synthetic
  source 1..21: mol_type = other DNA
tgctctcaatttccatttata

SEQ ID NO: 408 - DNA, 21 bp, synthetic construct
  misc_feature 1..21: note = Synthetic
  source 1..21: mol_type = other DNA
gctctcaatttccatttatat

SEQ ID NO: 409 - DNA, 21 bp, synthetic construct
  misc_feature 1..21: note = Synthetic
  source 1..21: mol_type = other DNA
tccttcagcactttgaatata

SEQ ID NO: 410 - DNA, 21 bp, synthetic construct
  misc_feature 1..21: note = Synthetic
  source 1..21: mol_type = other DNA
tcagctattacttccttaaat

SEQ ID NO: 411 - DNA, 21 bp, synthetic construct
  misc_feature 1..21: note = Synthetic
  source 1..21: mol_type = other DNA
cagctattacttccttaaata

SEQ ID NO: 412 - DNA, 21 bp, synthetic construct
  misc_feature 1..21: note = Synthetic
  source 1..21: mol_type = other DNA
tccttaaggacctcctattat

SEQ ID NO: 413 - DNA, 21 bp, synthetic construct
  misc_feature 1..21: note = Synthetic
  source 1..21: mol_type = other DNA
gcttgacctctaaacatataa

SEQ ID NO: 414 - DNA, 21 bp, synthetic construct
  misc_feature 1..21: note = Synthetic
  source 1..21: mol_type = other DNA
cttgacctctaaacatataaa

SEQ ID NO: 415 - DNA, 21 bp, synthetic construct
  misc_feature 1..21: note = Synthetic
  source 1..21: mol_type = other DNA
accaataccttgtgtaataaa

SEQ ID NO: 416 - DNA, 21 bp, synthetic construct
  misc_feature 1..21: note = Synthetic
  source 1..21: mol_type = other DNA
ttgacactggctctctttata

SEQ ID NO: 417 - DNA, 21 bp, synthetic construct
  misc_feature 1..21: note = Synthetic
  source 1..21: mol_type = other DNA
tgacactggctctctttataa

SEQ ID NO: 418 - DNA, 21 bp, synthetic construct
  misc_feature 1..21: note = Synthetic
  source 1..21: mol_type = other DNA
cagaagatgtgtttgataata

SEQ ID NO: 419 - DNA, 21 bp, synthetic construct
  misc_feature 1..21: note = Synthetic
  source 1..21: mol_type = other DNA
gtttgacgtgaagagtttaaa

SEQ ID NO: 420 - DNA, 21 bp, synthetic construct
  misc_feature 1..21: note = Synthetic
  source 1..21: mol_type = other DNA
ctctgagcttcagtgaattat

SEQ ID NO: 421 - DNA, 21 bp, synthetic construct
  misc_feature 1..21: note = Synthetic
  source 1..21: mol_type = other DNA
agggttgaatgctggatttaa

SEQ ID NO: 422 - DNA, 21 bp, synthetic construct
  misc_feature 1..21: note = Synthetic
  source 1..21: mol_type = other DNA
gggttgaatgctggatttaaa

SEQ ID NO: 423 - DNA, 21 bp, synthetic construct
  misc_feature 1..21: note = Synthetic
  source 1..21: mol_type = other DNA
ggttgaatgctggatttaaat

SEQ ID NO: 424 - DNA, 21 bp, synthetic construct
  misc_feature 1..21: note = Synthetic
  source 1..21: mol_type = other DNA
gttgaatgctggatttaaata

SEQ ID NO: 425 - DNA, 21 bp, synthetic construct
  misc_feature 1..21: note = Synthetic
  source 1..21: mol_type = other DNA
agtgaaagcaaagagaataaa

SEQ ID NO: 426 - DNA, 21 bp, synthetic construct
  misc_feature 1..21: note = Synthetic
  source 1..21: mol_type = other DNA
catgatgttccaaactttaaa

SEQ ID NO: 427 - DNA, 21 bp, synthetic construct
  misc_feature 1..21: note = Synthetic
  source 1..21: mol_type = other DNA
aggtggccaagggccttaata

SEQ ID NO: 428 - DNA, 21 bp, synthetic construct
  misc_feature 1..21: note = Synthetic
  source 1..21: mol_type = other DNA
gcccagctggctctgtaataa

SEQ ID NO: 429 - DNA, 21 bp, synthetic construct
  misc_feature 1..21: note = Synthetic
  source 1..21: mol_type = other DNA
cccagctggctctgtaataaa

SEQ ID NO: 430 - DNA, 21 bp, synthetic construct
  misc_feature 1..21: note = Synthetic
  source 1..21: mol_type = other DNA
ccagctggctctgtaataaat

SEQ ID NO: 431 - DNA, 21 bp, synthetic construct
  misc_feature 1..21: note = Synthetic
  source 1..21: mol_type = other DNA
tggtgtcatcgggccatattt

SEQ ID NO: 432 - DNA, 21 bp, synthetic construct
  misc_feature 1..21: note = Synthetic
  source 1..21: mol_type = other DNA
taactaggtcaacagaatatt

SEQ ID NO: 433 - DNA, 21 bp, synthetic construct
  misc_feature 1..21: note = Synthetic
  source 1..21: mol_type = other DNA
gcaaagttaatcctcatataa

SEQ ID NO: 434 - DNA, 21 bp, synthetic construct
  misc_feature 1..21: note = Synthetic
  source 1..21: mol_type = other DNA
gcagcatctgttctgattaaa

SEQ ID NO: 435 - DNA, 21 bp, synthetic construct
  misc_feature 1..21: note = Synthetic
  source 1..21: mol_type = other DNA
cagcatctgttctgattaaat

SEQ ID NO: 436 - DNA, 21 bp, synthetic construct
  misc_feature 1..21: note = Synthetic
  source 1..21: mol_type = other DNA
agcatctgttctgattaaata

SEQ ID NO: 437 - DNA, 21 bp, synthetic construct
  misc_feature 1..21: note = Synthetic
  source 1..21: mol_type = other DNA
gcatctgttctgattaaatat

SEQ ID NO: 438 - DNA, 21 bp, synthetic construct
  misc_feature 1..21: note = Synthetic
  source 1..21: mol_type = other DNA
ctgcacaactgaccctttatt

SEQ ID NO: 439 - DNA, 21 bp, synthetic construct
  misc_feature 1..21: note = Synthetic
  source 1..21: mol_type = other DNA
tgcacaactgaccctttatta

SEQ ID NO: 440 - DNA, 21 bp, synthetic construct
  misc_feature 1..21: note = Synthetic
  source 1..21: mol_type = other DNA
tgatggctttcctactattta

SEQ ID NO: 441 - DNA, 21 bp, synthetic construct
  misc_feature 1..21: note = Synthetic
  source 1..21: mol_type = other DNA
gatggctttcctactatttaa

SEQ ID NO: 442 - DNA, 21 bp, synthetic construct
  misc_feature 1..21: note = Synthetic
  source 1..21: mol_type = other DNA
agctcagccatctggtttaaa

SEQ ID NO: 443 - DNA, 21 bp, synthetic construct
  misc_feature 1..21: note = Synthetic
  source 1..21: mol_type = other DNA
ctgactgccttagactaatat

SEQ ID NO: 444 - DNA, 21 bp, synthetic construct
  misc_feature 1..21: note = Synthetic
  source 1..21: mol_type = other DNA
tgactgccttagactaatata

SEQ ID NO: 445 - DNA, 21 bp, synthetic construct
  misc_feature 1..21: note = Synthetic
  source 1..21: mol_type = other DNA
cagcatttgacaaagaaataa

SEQ ID NO: 446 - DNA, 21 bp, synthetic construct
  misc_feature 1..21: note = Synthetic
  source 1..21: mol_type = other DNA
aggatatggatgatgtatata

SEQ ID NO: 447 - DNA, 21 bp, synthetic construct
  misc_feature 1..21: note = Synthetic
  source 1..21: mol_type = other DNA
gtcgaccctaagaagataata

SEQ ID NO: 448 - DNA, 21 bp, synthetic construct
  misc_feature 1..21: note = Synthetic
  source 1..21: mol_type = other DNA
agaaacctgaacaagtaataa

SEQ ID NO: 449 - DNA, 21 bp, synthetic construct
  misc_feature 1..21: note = Synthetic
  source 1..21: mol_type = other DNA
gaaacctgaacaagtaataat

SEQ ID NO: 450 - DNA, 21 bp, synthetic construct
  misc_feature 1..21: note = Synthetic
  source 1..21: mol_type = other DNA
agtcacacaacagactaaatt

SEQ ID NO: 451 - DNA, 21 bp, synthetic construct
  misc_feature 1..21: note = Synthetic
  source 1..21: mol_type = other DNA
gtcacacaacagactaaattt

SEQ ID NO: 452 - DNA, 21 bp, synthetic construct
  misc_feature 1..21: note = Synthetic
  source 1..21: mol_type = other DNA
agcgagagtgtcctcatttat

SEQ ID NO: 453 - DNA, 21 bp, synthetic construct
  misc_feature 1..21: note = Synthetic
  source 1..21: mol_type = other DNA
gcgagagtgtcctcatttata

SEQ ID NO: 454 - DNA, 21 bp, synthetic construct
  misc_feature 1..21: note = Synthetic
  source 1..21: mol_type = other DNA
cgagagtgtcctcatttatat

SEQ ID NO: 455 - DNA, 21 bp, synthetic construct
  misc_feature 1..21: note = Synthetic
  source 1..21: mol_type = other DNA
atcctctatctctgttaataa

SEQ ID NO: 456 - DNA, 21 bp, synthetic construct
  misc_feature 1..21: note = Synthetic
  source 1..21: mol_type = other DNA
agcaagtgctattccatattt

SEQ ID NO: 457 - DNA, 21 bp, synthetic construct
  misc_feature 1..21: note = Synthetic
  source 1..21: mol_type = other DNA
ctggagtccatggagaaatat

SEQ ID NO: 458 - DNA, 21 bp, synthetic construct
  misc_feature 1..21: note = Synthetic
  source 1..21: mol_type = other DNA
cctccaaggacatactttata

SEQ ID NO: 459 - DNA, 21 bp, synthetic construct
  misc_feature 1..21: note = Synthetic
  source 1..21: mol_type = other DNA
ctccaaggacatactttataa

SEQ ID NO: 460 - DNA, 21 bp, synthetic construct
  misc_feature 1..21: note = Synthetic
  source 1..21: mol_type = other DNA
tccaaggacatactttataat

SEQ ID NO: 461 - DNA, 21 bp, synthetic construct
  misc_feature 1..21: note = Synthetic
  source 1..21: mol_type = other DNA
ccaaggacatactttataata

SEQ ID NO: 462 - DNA, 21 bp, synthetic construct
  misc_feature 1..21: note = Synthetic
  source 1..21: mol_type = other DNA
gtgagggcaatggtgaatata

SEQ ID NO: 463 - DNA, 21 bp, synthetic construct
  misc_feature 1..21: note = Synthetic
  source 1..21: mol_type = other DNA
tgagggcaatggtgaatataa

SEQ ID NO: 464 - DNA, 21 bp, synthetic construct
  misc_feature 1..21: note = Synthetic
  source 1..21: mol_type = other DNA
cgaaaggcaagcttcttaaat

SEQ ID NO: 465 - DNA, 21 bp, synthetic construct
  misc_feature 1..21: note = Synthetic
  source 1..21: mol_type = other DNA
gaaaggcaagcttcttaaata

SEQ ID NO: 466 - DNA, 21 bp, synthetic construct
  misc_feature 1..21: note = Synthetic
  source 1..21: mol_type = other DNA
ggcaagcaaggcaggatttaa

SEQ ID NO: 467 - DNA, 21 bp, synthetic construct
  misc_feature 1..21: note = Synthetic
  source 1..21: mol_type = other DNA
acaggaacctggctctaattt

SEQ ID NO: 468 - DNA, 21 bp, synthetic construct
  misc_feature 1..21: note = Synthetic
  source 1..21: mol_type = other DNA
gggacctcctgacatttaatt

SEQ ID NO: 469 - DNA, 21 bp, synthetic construct
  misc_feature 1..21: note = Synthetic
  source 1..21: mol_type = other DNA
ggacctcctgacatttaatta

SEQ ID NO: 470 - DNA, 21 bp, synthetic construct
  misc_feature 1..21: note = Synthetic
  source 1..21: mol_type = other DNA
gacctcctgacatttaattaa

SEQ ID NO: 471 - DNA, 21 bp, synthetic construct
  misc_feature 1..21: note = Synthetic
  source 1..21: mol_type = other DNA
gtgggagcttgttacatatat

SEQ ID NO: 472 - DNA, 21 bp, synthetic construct
  misc_feature 1..21: note = Synthetic
  source 1..21: mol_type = other DNA
ctaagtcttctcatctatata

SEQ ID NO: 473 - DNA, 21 bp, synthetic construct
  misc_feature 1..21: note = Synthetic
  source 1..21: mol_type = other DNA
ttaggtggtcatgagataaat

SEQ ID NO: 474 - DNA, 21 bp, synthetic construct
  misc_feature 1..21: note = Synthetic
  source 1..21: mol_type = other DNA
taggtggtcatgagataaata

SEQ ID NO: 475 - DNA, 21 bp, synthetic construct
  misc_feature 1..21: note = Synthetic
  source 1..21: mol_type = other DNA
aggtggtcatgagataaatat

SEQ ID NO: 476 - DNA, 21 bp, synthetic construct
  misc_feature 1..21: note = Synthetic
  source 1..21: mol_type = other DNA
ggtggtcatgagataaatata

SEQ ID NO: 477 - DNA, 21 bp, synthetic construct
  misc_feature 1..21: note = Synthetic
  source 1..21: mol_type = other DNA
gtggtcatgagataaatatat

SEQ ID NO: 478 - DNA, 21 bp, synthetic construct
  misc_feature 1..21: note = Synthetic
  source 1..21: mol_type = other DNA
cacaaagcacatggctaaata

SEQ ID NO: 479 - DNA, 21 bp, synthetic construct
  misc_feature 1..21: note = Synthetic
  source 1..21: mol_type = other DNA
acaaagcacatggctaaataa

SEQ ID NO: 480 - DNA, 21 bp, synthetic construct
  misc_feature 1..21: note = Synthetic
  source 1..21: mol_type = other DNA
cggttatgtagaagctattta

SEQ ID NO: 481 - DNA, 21 bp, synthetic construct
  misc_feature 1..21: note = Synthetic
  source 1..21: mol_type = other DNA
ggttatgtagaagctatttaa

SEQ ID NO: 482 - DNA, 21 bp, synthetic construct
  misc_feature 1..21: note = Synthetic
  source 1..21: mol_type = other DNA
agccacccttaattgaataat

SEQ ID NO: 483 - DNA, 21 bp, synthetic construct
  misc_feature 1..21: note = Synthetic
  source 1..21: mol_type = other DNA
actaccctttactggtaattt

SEQ ID NO: 484 - DNA, 21 bp, synthetic construct
  misc_feature 1..21: note = Synthetic
  source 1..21: mol_type = other DNA
taataggcactagagaataat

SEQ ID NO: 485 - DNA, 21 bp, synthetic construct
  misc_feature 1..21: note = Synthetic
  source 1..21: mol_type = other DNA
atttgggcccaagtgtatatt

SEQ ID NO: 486 - DNA, 21 bp, synthetic construct
  misc_feature 1..21: note = Synthetic
  source 1..21: mol_type = other DNA
tttgggcccaagtgtatattt

SEQ ID NO: 487 - DNA, 21 bp, synthetic construct
  misc_feature 1..21: note = Synthetic
  source 1..21: mol_type = other DNA
ttgggcccaagtgtatattta

SEQ ID NO: 488 - DNA, 21 bp, synthetic construct
  misc_feature 1..21: note = Synthetic
  source 1..21: mol_type = other DNA
tgcctgctgtgggtgattatt

SEQ ID NO: 489 - DNA, 21 bp, synthetic construct
  misc_feature 1..21: note = Synthetic
  source 1..21: mol_type = other DNA
gcctgctgtgggtgattattt

SEQ ID NO: 490 - DNA, 21 bp, synthetic construct
  misc_feature 1..21: note = Synthetic
  source 1..21: mol_type = other DNA
gggatctttagggacataatt

SEQ ID NO: 491 - DNA, 21 bp, synthetic construct
  misc_feature 1..21: note = Synthetic
  source 1..21: mol_type = other DNA
gatgacactacgtagatatat

SEQ ID NO: 492 - DNA, 21 bp, synthetic construct
  misc_feature 1..21: note = Synthetic
  source 1..21: mol_type = other DNA
tgctctttcagtttgtaataa

SEQ ID NO: 493 - DNA, 21 bp, synthetic construct
  misc_feature 1..21: note = Synthetic
  source 1..21: mol_type = other DNA
gctctttcagtttgtaataaa

SEQ ID NO: 494 - DNA, 21 bp, synthetic construct
  misc_feature 1..21: note = Synthetic
  source 1..21: mol_type = other DNA
gtctttccatgggtctaattt

SEQ ID NO: 495 - DNA, 21 bp, synthetic construct
  misc_feature 1..21: note = Synthetic
  source 1..21: mol_type = other DNA
tctttccatgggtctaattta

SEQ ID NO: 496 - DNA, 21 bp, synthetic construct
  misc_feature 1..21: note = Synthetic
  source 1..21: mol_type = other DNA
atcagtttctctaggtattaa

SEQ ID NO: 497 - DNA, 21 bp, synthetic construct
  misc_feature 1..21: note = Synthetic
  source 1..21: mol_type = other DNA
tcagtttctctaggtattaaa

SEQ ID NO: 498 - DNA, 21 bp, synthetic construct
  misc_feature 1..21: note = Synthetic
  source 1..21: mol_type = other DNA
aggccaaactctctcttaatt

SEQ ID NO: 499 - DNA, 21 bp, synthetic construct
  misc_feature 1..21: note = Synthetic
  source 1..21: mol_type = other DNA
cttacccagtcactgttaatt

SEQ ID NO: 500 - DNA, 21 bp, synthetic construct
  misc_feature 1..21: note = Synthetic
  source 1..21: mol_type = other DNA
ttacccagtcactgttaatta

SEQ ID NO: 501 - DNA, 21 bp, synthetic construct
  misc_feature 1..21: note = Synthetic
  source 1..21: mol_type = other DNA
acccagtcactgttaattatt

SEQ ID NO: 502 - DNA, 21 bp, synthetic construct
  misc_feature 1..21: note = Synthetic
  source 1..21: mol_type = other DNA
cccagtcactgttaattatta

SEQ ID NO: 503 - DNA, 21 bp, synthetic construct
  misc_feature 1..21: note = Synthetic
  source 1..21: mol_type = other DNA
ctcaacagcttcaacattata

SEQ ID NO: 504 - DNA, 21 bp, synthetic construct
  misc_feature 1..21: note = Synthetic
  source 1..21: mol_type = other DNA
tcaacagcttcaacattatat

SEQ ID NO: 505 - DNA, 21 bp, synthetic construct
  misc_feature 1..21: note = Synthetic
  source 1..21: mol_type = other DNA
atacatcttgactccattatt

SEQ ID NO: 506 - DNA, 21 bp, synthetic construct
  misc_feature 1..21: note = Synthetic
  source 1..21: mol_type = other DNA
cccaaactggacagatatata

SEQ ID NO: 507 - DNA, 21 bp, synthetic construct
  misc_feature 1..21: note = Synthetic
  source 1..21: mol_type = other DNA
ccaaactggacagatatataa

SEQ ID NO: 508 - DNA, 21 bp, synthetic construct
  misc_feature 1..21: note = Synthetic
  source 1..21: mol_type = other DNA
caaactggacagatatataat

SEQ ID NO: 509 - DNA, 137350 bp, Homo sapiens
  source 1..137350: mol_type = genomic DNA
caatgagaataaactcactgtagctccatgctacaacatggatacatctcaaaaacaattttgatgaaagaaggtggacacaaaagaatacatgctgtatgactccttttacataatgttcaaaaaacaagaaaaactaaaccatagtatttagcaattcaaacttagatggcaaaagtataaagaaatgtagggaaattattttcgtaaatgtctcctctagggaaaagggaggattaagtcaagctcatccatcactccatactggcccttcctcgccagtgatctcacatgtttattccaacaagagagctggagaaactgtgtttgtgctatatcaacaattctcagtgaatgtcttgttgactgggaaagcaatgtgggtcctacttttctttctttcattttgtaaaaccatttctgatgagcatgaatgagtcctccatttcttaagtaactgacagcacaggtttgatatcagcaatcaaagatctgccaggccttgtttatcatgcctcacctctgttgccacatgtttcccagggatcgattgaatgtgactatgagtagctctccatgtacaccatctagtgtcacttctgggacttgggggtttctgacttacagtcctcttccctatcatttatttacatattcagtaaataatcatgaacacctaccttatgccagacagtgttctgggtgctaaatatatagtagtagaagagataaagtttatgttttcatagaactttcaattcagtacatgagatggaaaggtggacggtgcctgtagtcccagctacccgggaggctgaagtgagaggactgcttgagcccaggaatttgagaccagcctggacaacgtagtgagaccctgtctctattaaagtttttttaaattactgatataatttaaggtagtggtgagtactagaaaaaatgtaccaggagaaaaaaacagaagcaaaagtacatgtggccaatactattttgatatgtagaggccaggaaaagcttctctgaagaggtaaacacaaagttgaaggatgtaccatcacagagagatggaagttttccataatttccttgcatatactatggtgtgacttcattttttcacccacgtctgttaggctgctcagataaactgctttttttttaaacgagatctaaagaccacgtacattcttcatagtccctgaattaagtattacccaaaccatagctcatgggccacctacctgttaatttaatgtctatgtatgccttcactctacaatggcaggcttaagcagctgggacagagaccacataacccagaaagcctaaaacttttattctctggccctttacagcagcagtccctaaactttttggcaccagggactggtttcccagaagacaatttttcctcctactgggggtgagggatggtttcaggatgattcaagcgcattactcttactgtgtactttatttctattattattacattgtaacatataaggaaataattatacagttcaccataatgtagaatcagtgggagccatgagcttgttttcctgaaactagacagtcccatctaggggtgatgggagacgtgacagatcatcaggcattagattctcataaggagtgtgcaacctagatcccttgcatgtgcagttcacaatagggttcgtgctcctatgagaatctaatgctgccactgatctgacaggaggcggagctcagtaatgtgagtgatgggcagcagctgtaaatacagatgaaacttccttgacttccctgccctcgcctcctgctgtgcagcaatttccaacaggccacgacaggggttggggacccctgctttacaggaaataatgtgctgatccctggtttagataattgattttgaaatttgattctatttaaaactattagttttcctgtaagctcttctttagctgcatttaactggttttaatatgatatactgcagtatcattcgattcaaactattttctaagattcgaagatttgctgtaacttatccttttaccaatgggttatttagaagtgtgctgcttaattgctaagctattggaaatttgtctacttacctttctgttcttaattgtcagtttaaattcactgtgatgtggtcagagaatacaacctaaatgttatcaatcatttgaaaaaattttaggacttaaattaggggccagcatacagtatactttggtaactgttccctgtgtaaataaaaagaaaatctgatctagaactgttaactgtggtatcttacatagtcaacttgattaatttaatgaatcatgttgttcaaatattttatattcctgttgatgctttgacagctgttctaatactgagaagtgtgtgtgaaaatctccaactattattatatgattttgatgtttcctcatctaattctgttacactttgaaatataaattttgaagctattaggtgcatataggtgaaatattacatacagaatattttatgatttttctgtttaattgacagttttactattacaaaatgttcttgtgttcttctagtgatttttcttgctttaaagtctattaacttgatattatataataatcaactttcttatagctattgtttgcatgttataacgtctatccttttactctccatttcactgtgttcttatatttaaactgcctttcttatcaatagcacatacttgagtcttgtttttgtatgcagtgtgacaatctgtctttaattgaaatgtttagttatacatatacaatataacaaataaaatagatggatttatatctgcagtcttgctatttattttctattaactccatctattttcatttcatattttttctcttctgccttcttttgaataatcagcttttttgctattctattattctcctctattagctattcttgaatattttaataattagctagagattcaatacaaactccttatgttctacagtctaccttaagctagttgtttttaccacctcctgagggtgcaagaactttacaacacatgtaatcaattcactgtcctcagtctttgtgctattgctggcatatatttttacttctccatgttatatatttcacaatacatcataattatttttaacagttatttgtttatatttttcagatatatttacccaatccagtgctctttatttctctttcttgcagttttatgctttcatctgggataactttctttcagcctgaagagtctattttaaaattttttctagaaaaaatctgctggccatgactattttcaacttatttttgtctaaaacatttttattttacatttatttatgaagaatattttctctgagtataaaatgttaggttggctgctttttttatttcaggtttcagagatgcctttctattttctcctggcacctataatttctgataaaaagtcagctgtaagttttattgttgttcctttaaaggttatgtgtctcatttctctgaccatatttagggtcatctcattctctttggttttcagcagtctgactgtggtgtgcttcagtggggttttcttttactctacgtaagattcaccagtctgcagtctgtgagttgatagccattcaacaaaagaaaagaacagcagctaccattgaaatctccacctgattagacttacaataatccacactgtcattctctaatatccatctgattttttgcacaagccaaacaaccaagttaagtgggagaggtagtagaaagcaacacccctatgtcttcccaattttaggtggtggcactaaatcactaatattctccttgaggcatggtattgcttttggtttatttttttcaaatagaggactacgttcataggtttatatcacgagagtccctcattctatgggtcagtggaacaactgaggatcttgtcagataacaagtatgtctattacaactcacattcaggatctgaggaattaacaggatggctcatggacctactggacccattgagaaatgatgtcaactgatgttcacaagccctcatcaggcttgaaggaccagagtgtctttatggtcgccaggaattattacatgttagctcagtgacagtgcccccaaatccctgaaagatctggatatttcctttcccttggtgaacagttattctgctaaacgatcgcgtagctccttcaggagggctagaaggaaaatatacagcatgaacttattgggttatcctcagctgtacccagactcaccttcactcacatgactctgtgtctatgaactgactgaggtttgggatttggcaaggggctgtgaatctctattgaggtggcttaagtcaagctttggtttactcagaactagaattgtacttatctaaaaggacacagtaaactgattacctatttcagtccttgggacttcatgaccaattcacctgtcaaagatccctgtaggctaaactattcagattgctactctagctcttttgtgcaagatttcacctcctaaattagactgcacgtggcctgcttaatgacttgaaccacaatctcttaaatcaaagtctttccttcaaaagctactttagtccagctgactacaaaagatcatttaactgtttggtcactgtgtgccatggaccactgtcccactgccagcactacttagatacttactgttttcaaatccatttcagacactagtcccagatttctatttctttaaggatcttttgtcatcaattctgtgtcaataatacttgttggttggagacaacagaatccatagtaactagtctaggcaggcatgtcagatgatcctagatgacccttatgctcaagtatttgccggaaggactcacaggactcagaaaagctcttacactcatggttatagttcattacagcaaatggatacagattaaaatcagcaaagggaaaaggtacatgggatgaaatccaggagaaactaggtccaggcttccagatatcttctctgctggagtcacatggagatacacttaattgtctctgcaataatgtgtaacacatatgaagtagcttcctggttggcaagacttcataaattttgccattatttgaagataacccacaccttggtgtccagggttttcacaggtattagtggtcagtcacataggcatatagtacctgtatgacttctattccagccaccgcaaacttctcctctccctgctggctcctgagccaaaaacaggcatttaccataaatcactttgttaggatgaacttatctggccaaactgatacagcatgacccacagcctcaggtatataaaacactctcatcaggcagaatgtttcagggtctcagaggttagtcaggggctagtcaaggaccaatcctgaaggcaggcctttttttttttttttttttttttttgagacggggtcttgctctgtcgcccaggctggagtgcagtggcatgatctcagctcactgcaagctctgcctcccaggttcacgccattctcctgcctcagcctcctgagtagctgggactacaggcacccgccaccatgcccagctaattttttgtatttttagtagagactgggtttcaccgtattagccaggatggtctcgatctcctgacctggtgttccacctgcctcagcctcccaaagtgctgggattacaggcgtgagccaccatgcccagccaaggcaggcctttttttggaacctacagggtttgaccaacccaggactgctgagttaaccctttcctgtgcagcaagaaaagatttatttaaaaaaaaaatggaaaatcacagtttaatcagaagggatgaaaagacagcttctagcttgtatttctgagaacatttcccagaaccatatcataaaactggcctgctaaggaagatgtctctgctaacccagaacaaggttaaattgcatgtggtttatggttatatatgggaatgccctggttcttatggaaaagatacagggataaagtgttacaaagtctgtaacttatgacaaatgggtccatagctggagataacagataaatggatagatagaacaaatatggccagatataaactaccaggacatctagctaaagaaaatatggttattgattatactatgctctaaacttttctttagatttaaaaaatgaccaagaaataaggaagtacaaaaaactatggcatgtttcatatgagaagtgtttgacataaaagggaaattgggaggctgggtgtggggctcacgcctgtaatcttagcactttgggaggctgaagtgggaggatcgcttgagcccaggagttcaagaccagtctgggtaacatagtgaaacctaatctctacttaaatatatatatataaaataaattgaaaaaaagagagaaataggaaaaaagctactgagaactagatagtaggtttagtaggtttgaaagtcatgaagaagttcagactgaaggacttctagatgtgaggagattacaatcaggtaataaaatatagtacagttgacacttggcacacatagaaatgccacagaaatatggaaaaaaaggaaaatatgattgcctttgggtgttaacctgttggcttattcacatatggaccaaagcagagtttgttataagtcacttaaaaaaaagagaaatggcttccaggcctgcaagatataatggaagcctgacatttctaaagaaaagggctagtcatcaatgaaaacactggataaaatcagactaggaaagtctggctgtaacttcctaggggatatttgcccaaaagcttcaacatactctcattcccgtcaatgatgcttcacggaagttgaatgtatcaggtatcaattgtccagttctttcagttcctgttctgtcacttccctttgtctagatttcaagcttcctaagaagcttctgttatgtggggaccctgtgtgtcttcttacagggaaaaaatgctctggtcacctgcataccagtcctttccagcattccagtccctctgatgggtacttttcaatttaaatcaataaactggcattccgattttataattccatctcttgagtttgttaaatgtgagtttgattggtgggctggcttaaatcagtttctaagcctgcaatcatgagggccagacctgaactctagtcaactcaccaagtttggctcgtttgttcatttgtgtggcaaatgcgattggtgccctggatcttcctactgtattgtatgccagtcctagtttcaattgccagctcctgcatttcttgcttagggctctctctgaccagctggagctgctgtgtcccaaccacagcaagccagccagatgaggataacaacattatacactcacaggataattgtgaagtgtgtgttctacactggctcccagagttcttcaacataagcaaattccagttgcccaaagtggacacttgattgataatacaccctttttggtttcctcccttccgtaactcacttactttcttaccagtgtttctagggatcacctcccaaataaattacttgcactgaaatcattgtctcaaggtctgttttcgtgagaaacacaaactaagacaacttcgtccattcattcatcatttattgagcttaatcaattattaataagtccttaaggcacaaaataagtccttagcaacatgttatctcttttctcatcacagtattatcccctttacaaatgaagaaatcaaggttgaaataaattaagtagctgactcagtatcacataatcaggacagagtgctggaaaggactgatggcagatgaagatggctgagactcccagatgcccagtcttgaatggagagtataaaaaatgagttttctgctggggtaataatttgagtatattataagattgctacgaagcaatgaaaaaagggattattgaatgaggaaacatttcaatgaaatatatttttgaaatttttgttttctaggaaagcaacatgctacaataagagcgcacacacatacacctgtgggtttaaatcctagcactactagtttttagctgtaagacatgggcaagttgcttacttactctaagctcaattttctaatccataaagtatataatatccaagaatgttgtatgaattaatgagatttatttaaacaactggcacctaatgagtgtacaaatttaatttgcagaaatgtactgtcctttcaggaactttgtggtaagtactgaaatatggatgcaatcagtactatgcttagcagtaacttaaaaaaagaagcaatataaaatttgtatgtcataaatgataactatattactatctttaaaaatcttggtctttccatatgtagtatgctaatttaatgttacaaaataaaagtaaaatcaataattttaaaaagtcagactttaagtataaaattttgtttgaagcaatgccaaagaatccacatagtaacatttatcattttgataacagaattttgtgttctgtatgtgggcctcaagcagtactaaaacaaaatagtgtatcaattataaatgatactcatgattcttccctagtcagatgtttttttcagttgagagattctgaagaaaaacaataagtgtagaatgtctgtaccatctttaatactaagatttaattgaatcaatgcatttccttagatttaaaaatcattttgttagagtctgtgttagaatcagtatgatacgtatttaaaatggaattagagatatggactgtagtaggaatgacttcaaataactcaaatgatgcgcatgtcagaaagaaacaatttctaagggctgcttcattgattcaagtggaactgttttattcagaacagctcagattagcagcagcagcagctggtgttcaatgaaatgcttcccttttccacctactctccagctgggttaagacaggcacaaagagattgggatgaccaaggtcaaagcatgatttgatagacttccaggtttttgctccagtaacttcttgtgtagtcttggctatagaggcaattcgtgaaattggtggttgaaaaagtcatagctttagaaggacttcatagatcatcttgttcaatatcttaattttatacttcatataccacggttctaattaaaatactttattgttgcatatttattggaattgacaagaagttaagatttcttacatttatttcatttgcctgatttttctgttcccactgaattgtataagcaggcactactgcactttgtatctctacttgacatagctgaaaaatgaaacattcaaagatacactactttattacatgttagctcagtaaaggggccctctattagacatgctggtctcagttcttaacttttacactcacaaagcccgattcactcattcaaaccttttttgaggagttaccaggttttagccattgagatagatactgggaacacaagaagtcaagggtgctctcctctctcaagaagcttaagagaagacagaagtgctaagcacatacatcgagtatgcggagggcacacagaggtataagcaatgaccctccttgagtagatctgaaaggcttctcagaggaggagcttgaacttgtctaaaaggtagaataaactgcatgcaaaagagctccaaatgactctcaagcttaagtattgcctaagcaatacattcatgtgggaagtgggagatggggctggaatggaggcagaggccacatccaatgggccgtttttggagtttagacattattcagaaggccatggtgagccactgatagagttaagaaaggcagcaaggtgataacacatgtttcttaagaaaacatactgtgatgaggacatttggcaggaaagtagtataatatgaaccagcattctctgaatgttggggttaaaatagtgaatgaggtaatatgatcttacccttctggaacttacagtctagttcagaaacacaacgggtctaaacactaagggtctaaattaaggtagtagcagtggaaatatacagaaggaaatggatgttatgataatgatgatgatgatattgcttattaccatttattgaacacctcccatgtgtcaggcactaaacacctcatatgcactatctttgtccattctccacacagtagctagagttattctatggatcttgtcataactctctcaaacccttccagaggcttcccacctcattcagcacaaatacctaagttcttaggaaaagctaaaaagtcctccaggctgtgacccactgtgacttctctgagctcatcttcaactcctcctttctctctctagccacaacgccttgctactgttcctagatgactctaggcacctttctgcttcagggccccctctgtctagaaggttgtttcctttgtatttccacagttcattcctcacttctgtcaagcctctgattaaacgaattcttttcaataagatcttccctggccactcggcatgacggaactcctgtagcactccctattccctttgccttgctttgtatttctccttaacatatcatattctgacataacacatatgtgttggttttcttcctccatggatcacaaactccatgaaggtagactgtttttgttcattgctgtattctcagcactgataacagtgctcgcacatatccattcaataaatatgtactgaatagatgaatgaaggattacaatggtcctatgaggatggtactattattttttcccactttcctaaagaggaaacagacaccaattgaggtactcaaggttgtatttctggtaactggcagaactgggatttgcgtggggcggggggaagcctgcctttgttgagttgtactatgtgcccagcattattctaagaactttatgtacattaacacttaatccctacacctccattttatagatgaggaaactaaatcagaaaggggtttacaatcttgcctggtatctgtgcacttgccagtaagttgggggcctgtgctctcagctactttgctacactaattctaaagccaattctttagagagatacaactttgcctcttaattgccaggagagtgagggaaaggaagaaatccaggattgctaccattattatgactgtggaccgccatagatggtagagtcaccaatcaagtcaaagaatgtaagatgagttggtttcattctcctctaactctagacttccacatccaactgcctcctgatatctttgcttggatattaagacacaaggtcagctgcagttaaagggatttagaataaccatggcagatcatagaagttagtagctgtcttacatcagagttgacctggtacacaggacctgcccaatgaagtcctcagggccccaacttgttgctctgcaatccctagaatgtttcccagtgtcttagtcttgtttatgctgctgtaacaaaatacctgaaactgggaaatttgtgaagaacagaaatatactttttcacagttctaaaggctgggaagtacaagctcaagacccccagcaggtttagtgtctggtgagggctgcagtttccaaggtgggaccttactgctatatcctctggagggggtgaatgctgtgtcctcgtgtggctaaagggatggaagggcaaaagaaggcctaagctagttccctccagtccttttacaaggtattaatccatccatgaggcagggccctcatgatttcatcagctgttagtatcatcacaatggggattaagtttcaacatgaattttgaaggggacaccatcattgaaaccacagcatctttcaacatggtttactaccccaactatttaatacatacattcatcagaaaaggacaaaagaaagggcaagtgacacccaatttcttttagaaaatgatctggaagttgcatacataatttctgctcacatcctactggccagaacttaagagatatggccacacttaagggtgtattataggcaaccatgtgtctggctagatgttctattatcatggataaagagcagaatgcataagagtcagctaggaagcagtttgtacaacagatatccaacaggtatctcaaatttaacattaccaaaactaaattcctgcttgttctcccaaaacctgctcctttgcactgttatcacatctcagtccatggtggaagacagaacaaactcaggggtttgatgttaatcatgttgagcttgaggtgcctgtagaacacccagtacaggtttccaataaggggttgaagatactgtgtctgatgcttctgactgagatctgtgttcaagattcagacttgattcctgactgaaataagtgatagaagagtatacgtaaatggtactgtcaaagattataacatggtaggcagagaaccaagaatggggagggaggattctgcacaggagacctgagaaagagtggcctaagacaaggcagaagagcagagaatgtaaggtgacagaaccaagggagcagtttccaggaggcagtggtcagcagtgtggaaggctgcagagagatcagatgttatgaaaactgagaagcaacctttcaatttggccattatgccacagtgaaattgatacaagcaatgtatacagtaaaatggagggagctgctaaatttctgtagttgaagaggaatttggaaggtaatagcaatctaagtatagacagcttttctaaatagaagggacaaagtagagggagaatttatgaacaggagtgggaatatttaagggagtaagagtcttgagtcaatctctcttccctcaagaaataaaaattacctgtagaaaggacagaaggatggcttctaccttaagactcgaggtcagaaagaatgcaaatttagacaggtctgcaggcataagggtgggggtatgtaggagttcatgcctgatgtgtgcagtactaaacaatgatacatatactactactactaccactactaagcactaacatttactgagcacttatcatatgccagtgttcttcatggagctaagtttgcttaacagtatttcttaaataatgggagctattcatatattgaaagcttagtgtttttcagaagttgagtgtgtaacatttggatgtggcctaaaaatagcaaccgtgacttctatcccaccatatctgaattttgtctcttccatgctcgacttatttctgatcctggggcatgctgagtagtaaaaaaagttgttcttgcattttgattcatctgttatgactggcatcaataagaaatcttcagcaataaaacagctctgtcaaaaaacaacttcaccagcaatttagggcaaaatgtttcctttaaaaaaaagtttgttggctgggcatggtggatcatgcctgtaatcccagcactttgggaggccgaggcgggcggatcacgaggtcaggagatcgagcccatcctggctaacacggtgaaaccccgtctctactaaaaatacaaaaaattagccaggcgtggtggcgggtgcctgtagtcccagctacttgggaggctgaggcaggagaatggcatgaacctgggaggtggagcttgcaatgagccaagattgcgccactgcactccagcatgggcgacggagcaagactctgtctcaaaaaaataataataataataaaataaaaagtttgttagtattagcagatacatattactaggtaccccccatgctcaatgaagtgttgggttactctgaaaaagtgtccaatcttacaggtgtgacttcctctggaactgcaagttcttgagggcagggatagagattgttcatcttttattgtttcccaaggtctggcacataaatagtagttcaagtagtccaacatatgacacataaatagttctgggtaaacattagctcaaagtttgctcaaggcattgatgcagatgcttataaaattaaattgctctttttactgtatacctgaaatgagaaaattattagaaactactgggtctcatttggactagaaaagtctgaagaaatcactgttcacttagtagatacaaagttgtttttctgggaaaaacacacctaaaaaaaaaaaaacacatgggtctgatatgggagaatttttttatttttcatataaacaggtgtatagggaagtggaacactgctagtatcttaaggattcctgaaagtatttatgttggtaagtttgatgagcaaacatttctaaatttgaaaatggataaaacaaaaagcacagtgaaatcttgaaagaattctaatactaatgtcacacataagaatacagatttaaaattaatgaactaaaacggatatacgtaatttctccaggaggaattaagatcctccttgatctcagtaaagcaatgaatagtcattctgacatctagctaaaagcagacttatttcccttcggtctttaatctttttggaactcagggatttccgccacactttgatacaccaggccattataaaattacacttaatttattgtcaatttctacttaaaaggaagaatacctataagtatctccatatgctagatatagtagaaatggtatgctttaattagtccaacaataaagtccttaaagatttacattctttgttgcctattttgaaaacttgcgaagataaatttttttaaagattataattcttactagtcctttccatgaaatataacagcaaatagtttccaaatgtggatgtgtaatgagaggaaatgaaaagtgagtaaagagctgagttaaaacatcttaaaatcgaatatggcttaatactatcattatcatcagttgtcctggaataacttaaattcttctagcttattgttggtttttcaatggttggttatagttgtgaggcggatacacacacacacccacacacttggctatacaaaaaagatgttccgagactgacagttaaaaattacactgctggccgagcactgtggctcatgcctgtagtcccaaaactttgggaggccgaggtgggaggatcacttgaggccaggagctcgagaccagcctgggcctaacatagcaagacaccatttctaagtttaaaaataaaaataaatttaaaaaagatacactgctatgtacgactgttgatataaaaaatctgaatactagacatgggtactcatctagtacttcaagggcttatcaacaaagttgcaagttgtaacactatgaattgttagtgatactcttttggctttgctagcaagtgttgtaaagctatacacacacacacacacacacacacacatacacacacacaccttttaaaatggtgaccctggtaccaaatatgactttaaatggatttaattttaatggctttaactacgttcagctgtcatatggatcaaaattagcctctatccagctggggtcaaccagggagccacttttcttaaccgacgacctactgaacgtcaacaactgcaggagacgggactttaccttcgtctctggtaaactagttgacacatcctgtgttggcaagaggcctaagtagatgaccttggtcctctaaaatctggcctgcactctcggggcacccctgcaacatctacaaaggcagctccagatagaaaagggttggggtcgaaaagccaataacggcaggcacctgccccgcctcggggctggggggctattccagcggcttcagctaactttcagagccattcgtttcccaacaaagtctgaggcgttcctctgctgggtacaccaaggggctctgcaaccctcctgggggggggggtgcccagagggcttccggaagtcccaggtttattctttcgggtcacagacagcagaaactaaaaagagggattaccctttctgtccagtcgcaagatggcgaccgagcctggtgggactccgaggggccgcaggccacctcctcttcccaatggcccgtgcgccggcggcgacggcaagcgggagggaggcggggccggcgaaggaaggaggggcggagcgcggcgccctcccgcgcgtcttggccccgccccacgtccccgcgtcccggcctggagccctcgcccggccgggcggcgcgcgctgcctgccgggatactcggcccgcccagccagtcctcccgtcttgcgccgcggccgcgagatccgtgtgtctcccaagatggtggcgctgggctcggggtgactacaggagacgacggggccttttcccttcgccaggacccgacacaccaggcttcgctcgctcgcgcacccctccgccgcgtagccatccgccagcgcgggcgcccgccatccgccgcctacttacgcttcacctctgccgacccggcgcgctcggctgcgggcggcggcgcctccttcggctcctcctcggaatagctcgcggcctgtagcccctggcaggagggcccctcagccccccggtgtggacaggcagcggcggctggcgacgaacgccgggatttcggcggccccggcgctccctttcccggcctcgttttccggataaggaagcgcgggtcccgcatgagccccggcggtggcggcagcgaaagagaacgaggcggtggcgggcggaggcggcgggcgagggcgactacgaccagtgaggcggccgccgcagcccaggcgcgggggcgacgacaggtcagtgttgccgcggcctgcgccaggcggcgctggctcccctccgtcactcggccggccttcggggcccgctgtggcgaggtcgacaccccccttccccgccccccgccgccgaggcgagtgtttgggggcgcgtggtccgaaggggctggtgccagaagtaggcccctggtggccgcggctgctgcagccgtaactgtcagtcctggctgagcgacggcgggagggttttgtcgcccgaggggacgcgagcgggcccggggcggggcgggacgtgcgaggcgtcgagatttgggcctcctaggagccaggctcttcgagccagccggggccccagacagggaagggcaggccctttccttcaaaggggagccctttctcggcgttttcaaggtttttggctctcttggggaagacatatttagccgtgtgcttggtgggttggggttttgggggtggattgatgggaagggagggcggatgaagtggtatgtcaagcccaagggttgtgcgcacaggttactctgtgttaccggccaccaggatttctgaagttgaacgtgagttattggctttgccagagactgctgtgttatatgcagacctgtatgcaagcagttggccttttttccccccccttttcagtgtagaaaatgaaaaggatgctttcctcatcttggtggtaaaggcttttgttggtaaaggtagaattgaatgtaccaaatgccttagtccgtaaaattttagaaataattttaatacagacactggtgaagcttggcaaccttgaaagagaatttagcgtctacattttttaaatgactttttatggatatgctaaattagtaacagtccaaaatctgtttgagattattaagtggcgagggtgctgttgaaaatgtaaactaatagcatatggggtttacagtgcacagttaacctcaatcatgaagaaatgtggatatgacccgtaattttggatcattttactgcctgcaatattgagagaagcagcaaattattacagttttttttgggagacgacctaaagtttaagaaatacaactgttgaaagttacctgtcagagacacaaaggtacccaatcaatcttgttgaataaattggacaagtgggataaggtgtttgtctcacacttctgatcaataagtactcttacttaagaagtgatttggtaaatcatgtaaaatttagaatttaggagagataagaaagttgtaacttggtgtgtatagtggaaatagctttgaaattagatcctgtttttaaatccaagctacttaccacatttttagtgaaatgaatacattattaatgatgctactgataagccctaaggatgaccagaagcccttttaagaaacactaatacagttgaccaaaaaataaaaaaggaaatagcttcaagaaataattttcaatctttgtgtatttataatatacacaggagaaataaaagaacagaatatgaagaggtaacttataatctttacaaaattagaatgtaaactatgctagtcttactgtgtcaatatttattaacctgcattagtttgctaagtagtatgctcactatcctctggacattgcccctgtccctgaggagcttaactttatacagagatacaaaatactgtgttttgatttcctttcagaggatttataagctacttatgttttatctgtcctaaattctgcccttttttttcccatggctgaaaaaataacttcctaagaaaggtacatactagtcagaggtgtggggaaaacgtgttctcatcccacttgtgcagtccatttaacaggatttattgggtacctacagtgtctttgacatgtaacatttaatcagttgtatttcttaaactttagataatttctatttcgatcatttctatttcaatctcctccctctcccctccaaaaaaatttactgtgtaaactgtgataatacactgtttctctcagtgtcagtcagtggaatgatccagatttataggtcatagcaaaatttttcatcccaagctcttgaaatgatgtttccaaatgtccattttcttaaatgactgctagttatgttctccaatcatgttacacatattttgtttagacttataattgtgtaggtctaaatgtagaaacattacattatgtacatatgcagctgctgaaagaaaattagttttctaatttttagatcggggtgaagacaaacttttagaagtgatctttcggtaaaatactggatgaagtcttaggtgctctttttatgttggttcaggacaaaagttttttgactgctttttgagttttcccctcactgccaaatgtaaggataaaatgtagtaacaagacatcttggtatgtatagtggtaatatctttggaattagaccctaattttaattctggccttattaatcttggtaagccacagttttgtcatctgtaaaatgggttggtgaaacttccccagagttgtgaggcttaaagtatgtaaaacgcctaacacgtaggcactcaataagtattaccttagaaatcttcctatccagtttttctgaatggtctggaagcaccttgtatgtagtggactacagcatgttatcctttatgttgtttatttcttttctcattagctatgaactttttaaggccaggaattcctagtgtttaccagagaatgtgttactagatgtcttagtctgttctggctgctattacaaaacaccacagacagcatggcttataaacaacagaagtttatttctcacggttctgggggctgagaagtccaagatcaaggtaccaacaaatttgacatcttagtgagggccctcttcccttgttcatagatagctagctgtcttttcaccattacctcatgtagtagaagagtagacggggctctatagggcctcttttatagaggcattaattccattaatgatggcaaagcttcccaaaggccccacctcttaatactatcaccttggaggttaggctttcaacatgaattttgggggacacattcagaccacagcactagagcaggcatgaattggttattggtaaatcagtaaatgatcaactaacatttattaggttcctcctgtggcttctatgagctgagctaagtacaggacccacaaagacaagaaagatgtctgtcttttttgaatgcttatagttgagtgactttctcacatttgtgtgtgtgtgtgtgtgtgtgtgtgtgtaaaaccctatcacataatggaacttcattttcccatgcccttccaatctaagagcagttctatggactcaatacctcctacccaccagctgctactgtaaagatttacttttttactccttttaaaaagttcaaccctctggcacatgcattacttcattccttggtcccagcccaccaccccagtatctggcacgtagtgtattagtatttgtgtgtgtgtgtgtgtgtgtgtgtgtgtgtgtgtgtgtgtgtgtgtgtgtatgtatatagaatttaaaatttttactccggcagttgtcttcaaatactctaattttggggggcatgattatcattctgtcttctggcagtacatggatagatgctacctgagtaggattgtctatgaggtcagctgctccagtcattttatacaagaggaaattggagtgcaaagctttaaaatgattttcttaaagtcacccaggtgtcagaaccagaaccagagtccatgtctctagttcaggactctgcacacattggcacatgttctgtcctaccttttgggatgaatagttatacttgctttgtgattactcacccacctttttggagacatatgagtgttttgtacactattaatagaaggcagccttcacaaaatgccatctgcagatctgttatttcttctaataaaggaaatgatgtaaaattaccttaaatagaaatagtgttaattcttctggagacatattccataatccaaacacgaaatgtttgaaatacttgaagatgccatataaataagtcctttgtaaaaagataatattaaaaatttttgttttatagagatgggggtctcactgtgttgcccaggctggtcttgaactcctcagcttttactttagcttcccagtgtgttgggattacaggcatgagccacaatacctggccaagtccttttttttaatcaaatgacttattaatacacagtttctttgccagcttttgttttcatttgctatcaaaaatgttgcttagtagtgctttgatctgagttatcaataacaggtaaatgccattatggataataattcaaaaagaagcttattaattattaggcctatctgagagtgaagtaaagttagcattttctttttgtttattttacttattgtttatttgtttagagacagggtctcgctgtgttgcccaagttggagtgcagtggtgctgtcataactcattgcagtctcaggctggagtgatcctcccatctcaccctcctgagtaggtgggattagcatatgccaccatgcctggctaatttttttattttttaatttttttgtggagatggggtcttgccgtgttcaggttggtttcaaactcctggtctcaagcggcttggcctcccaaggtgctaggattacaggtgtgagctaccatgcccagctgagcatttttaaaaaatactggtctttgtacatgagtcgttactatttgattctaagccttatgactgatatccctaaaaattatttataaaattttaagtgcatcagagtcattgaaatggaatgagcactgtcttttggtcttgaggttgttttaactagcttcgtaatggtcatgagcaggttatttagcttttgaagccttcgttccttcttttgtcaaatgaaagtgatagttgctttgttttaaaagagtatgcttttcaaacgtgatcattcttgaaaatgtagattaagagctttttagaggccaagtgccatggctcacgcctgtcatcccagcactttgggaagccgaggcgggcggatcacgagatcaggagatcgagaccatcctggccaacacggtgaaacctcatctctactaaaatacaaaaaaaaaattagccggacgtggtggcgcatgcctgtagtcccaactactcaggaggctgaggcaggggaaacgcttgaacacgggaggcggaggttgcagtgagccaagatcatgctactgcctggcgacagagtgagactgcctctcaaagaaaaaaaaaaaaactttttagaaacgttgttttggggaatttttagtgtgtgaattcattttaaatatgtgaactccatttgacatggacaatggatcttaatagtttgtgatacattattcagtgtattaatcaaaagtcaaatctcttttgtaatttgctgtaccagacctgttgtgttaatctgatcaatggcaagtgaatcataagattgacctgaaaggaaaagatatttttaggggacatagaatcatattggataagattattcctatagttcattttcaggaaatctgaacaggaatctcgtgtaaggaatcacatgtgaattatagaagagagtctgatgtaagactttatttattaaggtctgcatctgtgtttcaagtaagagcatagaggtagtatatgagccctgaacactcatacaagtaggataaaatttttcttaaaactgttggctggacagtgtagagtctaatgctgtgactcttaacagttttgtgtgttccagaagttcatctcctcaggatcaattgaggttagtctagcacagactttagttatagaaaccaaacatgagaaaaattttttgacccatgtcttcgtagcatgcaggcttggtagctctttcctagttctggatgtgtgcccgttgtatatactttcagcaattcaggatgttccttttcatatttaagacaagcactcttgcatacaaattacatagctgtgggctgaatgaagatgctaccattgctgtacttctacacaggttcttttcatgtaatgaaatcagaagggatggcttttccatttcaagcttctgttaataagtagtcttagaattgcctttgtgtactgtagccaacaccccaactcatatatttttataatctttgtgatgcagaataaattctgaaactttccacctcctaattaaatatagaaatactgaaaacaataaagcaccatgtattcactatggacatatgtcacattttgtcactttcttgcactcttgtaatccctctctaaagctcatgttaaactcatgttctttattttcttctcagatgattcctttggcactgcatgtaacagtacaggaatttcttcctttcgtacttccaacaaatacttttttgtctcttaacagatgtgaaccataatgctgttctttagaaagcaaaggatgaagtgcacagcagtgttttctatataaagctaatgggtcataaggggaaatatgggttatttccagcttgttaattgtcttaatacatttttcctaagaattctattgtgcctctgtggttatatgcaccaatattgataatcttacagcgtgccataaataaggatttttatattatctttgctaaatattttaagtttttgttttgggaattgaaaaaatcttttgtgcatctatggtatgtgatacgtatactaatggataaatcctgtttcacacaaagattaattgggtaacatttttctttcctagttttctgaagtcagtggtcatgttgtgtgttggtgattgaaaatatgctgtttaggctgggcgccttggctcactcctgtaatcccagcactttgggaggccgaggcacacggatcacctgaggtcaggagttcgagaccagcctggccaacatggtgaaaccctgtctctactaaaaatacaaaaattagctaggcgtggtggcacacgtctgtaatcccagttacttgggaggctgaggcacgagaatcgcttgaacctgggaggcagaggttgcagtgagctgagatcacaccattgcactccagcctgggcaagcaagatttcatctcaaaaaagaaaagaaaagaaaagaaaatatgctgtttaagatcattcaaattcagtttctaagaagtgtaaagttttcctaattacttaagctgcttcattctcattcccttttgtaacacattttgccatctctgtaacatgttgaacatgtttaaagaacagagtgggagaataggaagttacgtggataaagtttggtaatcaaggtaggaatacatcctgcttcacagaatgggaatatttctgaaaagttgactgtagtattgaagaacacttaagtgtagtgtattagacgaactgagctaattttggtcttttatacacataaatcaagatttctgcatttcaaatcatgcatatttctttaaatttattgctcagtgtactgtatttcaaatattgtaaaaatgaaaagatcttgtggtcttatttaactcagtatgggataagtccttcccaccttcccattcttattccctcttttaggctgaatgaaaggctgggggagtgaatttaaaaatagatggatctcagctggagaatgggttggtcacgtatctcaactgatattaaataatttcttaaatcctggattgaagattataagaaaacagtaaatgtttgctctcagttcatttctttgttttgttttttcatgtacagttttaggcacttgtaactgctggatgcatgaactactttctagttctttcaggcctacttttagatatctgctgtggggacagtcctcaacacttaaagtaattttaaatagtgataggcagaactaggggtcagttttactgcctatgcctgtgctgaatttgacatggcattcctaggaggggaggtggaacgagggcggtgcacacattgtccatattgctagtcagtgccagattttgagaaactggggctaatctctctcttaaatggtcaagatctaattccccaaagtgctgtttcagataacctagtatttgtccaaaattgccctgctacaatagtatttatcttatatatatgtattatatatataatatatatataatcttatatatatgtattatatatattatatatataatcttatatatgtattatatatataatatatatataatcttatatatatgtattatatatataatatatatataatcttatatatatgtattatatatataatatatatataatcttatatatatgtattatatatataatatatatataatcttatatatatgtattatatatataatatatatataatcttatatatatgtattatatatatataatacatataaatatatatatatatatatatataggtttttttttgtttttcctcctgagatgtagtctcgccctgttgcccaggctggtgtgcaatggtgcagtcttagctcactgcagcctccgcctcctgggttcaagtgattctcctgcctcagcctcctgagtagcagagactacaggtgtgcactaccacgcccggctaatttttgtatttttagtagatacagggtttcaccatgttggccatgctggtttcaaactcctaacctcaggtgatctgcctgcctcaacctctgaaagcactgggattacaggcatgagccaccacaccaatcctattacattcttaaaaaaatgtttgtttgtttgtttttgtagagatgaagtttcactatattacccagcctggtcttgactcctgggctcaagtgatcctcctgccttggcgtcccaaagtgctgggattatagacatgagccactgcgtccagcctgtatcatatttttattctgatatcttttgtaattttgtttttagtgaagtagggaggtttatattagataatcttccaaggtaattatcagcttggacattatatgctaatatgtcttttgtttgtttttaaaataagtgtatatgttttaattgtaaaatttttaaaacatccagaaaatgacatagcaagtgcctatttatccaccacctggaaatataaattatgtccatttttatcccttgggggaaaggatgcagttatataatcacttttaacaaccccaactgttcaagtcctgttacccgtctctacacaaagtaacaactgtcttgagattcttaagtaaatttcacatccttgttttatacttttactatacatgtgtacatgtatctgtaagtgatatgtggtgttggtttttgtattaaatttttacagacatgtataacgctgtctttcaatagcttgatttttttaaactcagtatcaagtttttgaggtgtatatgttgatgcgtgtacatgtagtttattcagtttcactgctgtatggcacttcattttatgcatagataaaatgtatgtgtcccactattgatggatattttgagttgttttctttttgctattataaataatgctgcagtgtttatttttacatgagtgtatgtgcaggtgttttgggaaaaaccgtagaagtgaaattgctggttactagatattggtaaattcttttcctaaatggatatgcccatgtatactttatcagcattatgtgggcatttctagcatcctcacccatacttgggattgtcagattttaaaatagttactaatttgaggcatgtgaaatgctatctcattttaatttacagttgcttgattattctgaatcgtaggtaaaaggataggaaatttattattttctgtatgttttaaatatctcaaaaataaatgtttttaaaggaaaggcacactatcgtacaacatttatgttgaaaaggaccatgaagatcatttcatccaaccttcttactttacagtgcagccaaagaaagttaccagaataagagtaaaatataaaatcctgcccattgtagtattctctggtagtatgagccttaatttttgatgtaaaaatcatgtctttttctttatgattgtgctttttgtttaagaaatcctctttttatattgtctgctaatagttgaggattttttgttactctttgttatttgcatttaggttcatctgaaaggagttaggattttttattttaaatatgaaaagctagttgttccagcattaagtattgaatttttaaaattttgtattgaggttaaatatacatacataatttaccatcttttttaagtgtacagttcatttttaatgattttttaagtgtacagttttttaagtgtacagttcagcaataataaatacagttatgttcttctgcctttgtcctctcactccccacttcggctgctagtaaccaccagtctatgcttatcttcttgagatatacttttttagcccatgtatgagtgagaacatgtggtatttgtctttctgtgtttggcttatttcacttaacataatggcttcttgttccatccgtgttcctgcaaattacagggttttattcttatttttatagctgagtagtattccactgtgtgtataaactacattttaaagtctattcgtcctaattattgaattctccccaccccacccacctttttttttttttttttttttccaggagacagggtctccctctcttccccaggctggagtgcagtgccatgatcatagctccctgcatccttgaaccccctggcctcaagcgatagtcttgcctcagcctcccaagtagctgggactgcaggcaggcacctccaggccctgctaagttttgtgggtttttttttttttttttttttttttttttgtagagatggggtcttcctatgttgcccactttggtttgttagttttagtagtttataattcctttgtattattgtgaacaaataacagttattttgaaatagaagagtgctttctatttaaaattttgctttgatagggttctgtctactttctgagccttaccttcctgtgcattcccctttggtcagtaccatcttaccacacaggctgcttttagtttcttgagttatccatcttccttctatcttatagggctcaacctaaatattactcagggaagccttccttgagagtctttctttttgcactcattggtttggggcttctttatattatactgttaattgaattctgagtcgtgtttatctcaactgagattaggtaatttattttgagaacagtaaatgtttatcatctgtcattctttttttcatgcccacattcagttatttgtaagtgctgaatgtataagtgttcatctacagataaggatcagttcttttttccccaactagaaatgttactccagttgataataacagctcaaacggaaatataaggcagaggtgagagagtcacttgaggccacgaattggagaccagcctgggcaacaaagtgagaccttatccctatgaaaaataaaattagcccagcttgggaggctgaggtggggggattgcttgaaccctggagttccaggctgcagtgagttgttatcacaccactgcactccagccgcggtgacagaccctgtcccttaaaaaaaaaaaaattataaccagccacattgaaatattttgtaagacattaatagaagactaaatgttctcatatgtttactgtttgtgttaagtacgtatggtttttagaacaacagttttctatcaagtgtggtctggttcacttagaattagatggggtagattttttgaaaagcgttttgaagttaaattagaagctaatggttttttgtcactaaatcaaattttgttttatgcgtttaagtgctacatttatttgataggcaggtgaatgtaacagattaaacttgaaaataatgtagctatgattccttcaaggaaaaacccacctacctttagttcttttagtatattgcattataagtcttttgtgtgtgcatatatagcaggcataaattggcataattctgggtttaattttaggctaggcgctatggaagtttgaactctaattacagtgtaaaaataacccagaggaaaatttcagaataataaatcattgggactatatggtacagatgtccctgttttcagattgtagaattgttgaccagtaggaatgctttatgattaaaagccaggaaattaaagtaattctgatgatttggtatttctaaagattttcatagatgacagttgagaactactctcagatgagattcttccattgctggtaagtcatgtaataaaacacttaagtaaatattttaatgaggaatgataacattttaatattggcacattgtgagtctaccttctgtagttagtaggtaaataagaaataaacataccttatttaaaaaaaaaatttcaaagaacccaagggaggggagagccaaggtgcacattagaaggagattacagtactcttccagtgcacagtggatgtgttgtgattccagagttctatgtatgtatgactgtgtagcccagatttaccagcatgggctcagttttaaatgttaggttaggctcataaatcatgaaaagttcctaggaattctaacatttttacatttccagaagagagcctcttgtattttgtggtagttgccccaccatccctcaaatattggtgtgtagaatctatttttgaggatgaggaaatacagaaaagaaaaataaaaagctatttttttttaagtataagaagaaatgaaaggtttctcttcgcttcatagccaattctaaaacttaacagttgttaagtgtgtttaactgtagagggaaaaaaatgcctggaaggaaattaccaatcaaatataaaatgtaagatagctgtaaaagtactgatatacatacagcttttttccttttggggaggcagtatttaaaaaaaatcctaccaaacatattctctaactttcaaaactgttaatagctttattgagataattcacatactataaaattcacccttttaaagtttttagtatattcacagttctgcagccatcaccactattgaattttagaacattttcatcatccccaaaataaatcctaacctgttagcagccattccccattctttcctggcctctggaaaccactaatctgctttgtctctatgaatttatttattctggatatttcatataaatgggaatcatacagtatgtgtccttttgtgtgtgggttcttttacttaccatggtattttcaaggtatgttcattgacatgtatgtctgttttttttgggtaacacacactcttgattactgtagctttgtagtaagtcttgaaaccaggaaatgtgagtattctaacttattctttttcaaaattgttttgggtctctgggtttctcaaatttccatagaattttagggttagcttgcaaaaaaatgcaactgggattttaataggacttggatttgtgttgaatctgtaggtctatttgggaagtattgctaccttaatactgtcttctcattcatgagcatgggatatctttccatttattaaggtgttctttaatttctttcatgttttgtagttttcagtatacaggtcttgtacttattactttaaaaatttatttctaagaattttatgttttgatggtattgcaaattgaattttttaaaatttcatttcaggtttttaattccttgtgcatagatttttgtgtactaaaacttgtgtttcatataatttttttttcctcttaactgtgttataggaatctttcaaatggttggctacatacacatccatctgattcacttagtaaacatttcttgaacacctactgtgtagtgggcctttttttctaggtcctggtagagacagcagtgtataaaacccactcagtcctttgctttcatggcacttaaatcctaaaaagagatcataaagataggtaaaatatatgttacatagtgctaaggagaagaaacaaataagggaagtgtttgcaggtgaaaaggatgatgggaaattttagatagggtaggtagagaaaacttcactgaaatggtaacttgggtgaagaactgagggagatgagagagctagccatgtggatatttgggggaagagcatttcaggcagagtgtagtgaatagggaaaccccaaagtgagagtgcctgatgtgtttgaagaactcgaggtgtggccagtgtagctttagaaaagtaattgatgggaaatagagagaatggagggtacagatattttgatcataataaggtgtttgatttttacctataagtgagatgggaagctattggaagtctttgagcagagtagtgaaaaagtatgacttatgtttagcaaggtcactctggttgctgtgttaatcatagactgaaaaaggggacaagggcagaaacagaccaattggtagaccattagtacagattaagacgtgactgaaatggacagagtggtcacagtgagggtggtgagaagtggttgaattcttgatatatgttgaaatagagtccataggatttggtagtagaccagatgaaagtgggaaataaagggaggagtaaaacatgatgccaaagttttttgaccagagcagctgagagaatggagttggataattcttataaattattgcattaaatctgtgcatcatatcacatgcccacttaactacaggtaattttaccatataagcttgtggggggaaaagaggaaaaacagaaaaaaggatggaaaagcatgttttaaaatataataaattaggccttaatgaaaaattgtccttgagaaacctgagatggaatactctaaatctgatgtgttctctcaatctttagaactgaactaggaattaactctgagagttcatgatgttatgagaccctttgcctgttttggaaattcggttgttttcaatatttgcttttgcaatgatggtacctgttattttttctgtaaggtagattcctacaaataagatgtcaggccattagatcatgtcaatctaaatatttgataggttttgccaattaccttttaaaaaataattttagcaagtttcattccttaaaatatattagggactgctcatttaccttgttttgattcatgtatatataaaatctgtttaagtctttataaataacctgggaaattgcctggttttttggtccatttctattgtggtgtttgttttttaaatttgtaagcattctttatatatcatttaattctttattataatttgcaaatattatctctacctacctttaattttatagtatctagtcatgtgaaattttaaagtttttatgtaggcaaatctgtccatcttttatggtttctgggtttcagcatgctaacaaagttgatataaatatgatctcatatatatattacaaaaactttttagagataggatctcactctgtcacccgggctggagcaaccatagctccctgcaacctcaaactcctgggctcaagtgatcctcctgcctcagcttcccaagtagctaagattatagacatgtgccactatagctagttggtttttttttggttggttggtttttttttttttttttggtagagaacgtatctcagtgtgttgcccagactggtctcaaacttctggcctcaagctatcctcccacctcagcctcccaaagtgctgggattacaggcatgagctactatacctggccaatctcttgtattttcttctgatacttttgtgattttactttatagactttaatttagaaagttagatatgagcattcagcatatttttggatatggtatgatatggtttagatctggattgcatttgtcatggtttgggaattatatctaccttttattttcttccacttgggtagctgattgtcttgtaaggcatcctttccccactgataatgccacctttttctatattaagttctcacaaactttgaaaaaaatcaagtttattgatgtataattgatacacaatgaaatgcacccattttaactatatagttttattgattttgataaatatatacactatcactgctactacaatcaagctttagaacaattgaattacctccaaaaagttccttcctgtctgtagtcatttctccctcttggccccaggcaaccactgatctgtttattagttttcctactctagaattgcatatccatggaatcaagtaacatgtactcttccatgcctggtttctttcacttggcatgtttttgagattcatccaatttgtgtttgtcacagatttttgtttgtttcgtttttttaaatacaatatgactataccacagttcattcattttctggtgaacatttaaattgttttcagttttaggctgttgcgaataaaggtactgttttgtgtgcatgtttttgtgtgaacatatgttttcatttttctagggtaaataccttaaattggaattactgggtcacatggtaagtttatgtttaaatgtataaggaactgccaaactattttccagatgattatactactttacattcccatcatcagtgtatgagagttccagttctccaaatgtcagcatttgatgttctcagtctttaattgcacacgttctgagaggtgtgtagtggtatcccattgcagtgtttttttttaagtggtactaaaatacacaacatcaaatataccattttaactatttttaagtgtacagttcagtgacataaagtacattcacattattctgcagccatcaccactatccatctctgaaactttttcatcttctcagtctgaaaccctatccattaaacagtaactcttcattgcctactctccctatctgctggtaatcactgttctacttcatatgtgaatttgactcttccaggtacttcatataagtggaatcatacgatttttgtccttttgtgtctggcacatttcatttaataataataatgtccttgcgttaggccattcttgcgttgccttaaatgaatatctgagactgggtaatttaaaaagaaaagaagtttatttggcccctggtgctgcaggctgtacaagcatggcgccaccatctgctcagcttctggggagccctcatggtgagtttactcatggtggaaggcaaagggggagcaggcgtgtcatatggcgagagcaggagcaagagagagggtggggaagtggcacaccctttttaaacgaccacatctgcgaactcactctgtatcccaaggagagcaccaagctatgagggattgctcccatgacccagttacctcccaccaggccccacccccaacattggggattatattttaacatgagatttgggcaggacaaatattcaaaccgtatcagtcatcaaagttcattcatgttgtagcatgtatccaaatttgattctttaaggttgagtaatatttcattgtgtgcatatatcactttttgttttatttatttatttgtcagtggacatttggactctttgtaacttttaactatgtgaatgatgctgctatgaacattggtatataagtatctgtttgagtcccttcttttaattcttttggttatgtacttaggagtggaattgctgggtcatgtggtaattgtatgtttaatttttttagagactgccatactgttttccacagtgactgcaccattttacattcccaccagcagtgaataagggttccaatttctctgcatcttttctaacacttggtaatttctgttttttatttaaaataattgccattctaatgggtgtaaaaagtcattatggtcttaatttacattttcttcatgactgctgttgagaagttttcttttgtgtgtctgttcaaatattttcacattcttttcagaacatatgttctttgtcagatagataaatgtatttcttctgtatccttactgattttctgtccacttactaattaccgagagaggaatgttgaaatcttcaactataattttgagtttatcttccttctgctctgttcattttttcttcatatgctttgaagcaaagtctttaaagttgtgtccttaggtgtgttcacatttaggactgttagatctccttgattaattgatgtctttatcattatgaaatgtctctgtttatcgctggtaatgttccttatctaaagtctactttgatattagtgtatccattcctttcttatgattaatttttgcacattatatcattttgctgtgtttttattttcagcttattggtcatttaatgtctttcttttagatagcatatagttgcatcttgctttcagattgactttttacgatcgttgctttttgattaagtccatttacatttattgcagttggcaatataattgtgtttaagacactatttgttttctatttgtctaatcttttcttccttttctgccttcttttcggttaatggagcatttttggaattttgtattgtctccattattagcttgtccctctgttttttttccccccagtgatacttctaggatttacaatatgcacctttaacttacctcaatctaccttcaaaaatattattcctcttaatgtataatgtaagaattttggaatagtatacatccatttactctccctcattctttttggtattatgatcttaacattttacttctatatgtgttatagattccacagcagcgtttgaaactatttctttcaaaggccagttgtcttacgaagaaacttaaaaactaagaataacattcctttatatttaccttcatatttaccatttttatttatttttctttgtggagattcctttttgtagatccaagtttgttgctggtgttattttccttttacttgaaaaaatttcttaaacatttctaaagtataggtctactggctacaaaatctttcagcttttgtttgtatggaaatgtctttatttcactcttcattttatcgtacaatttttcaaaccacacagactttaaaataattgtagaatataatttcacaacattttgttctatttgttattgtcttctatcttgtccatctatccatcctgtcttcttctagtgtctttttttatgcttttcaaagtaagttgcagttgttagtataccacacccttaacttgagcatgtatgttaactaaaacttagtagtttagtatttttaactagaatttactatgctgttccagttatttattgctgtgtaatacattgccccaaacttagtgccttaaatatcagcaactatttattttgctcatgaatctctcatttggtcagggattggcaaggagagcttgtctttgacaatacattgtaacaactgtttacatagcattacattgtatttggtattataagtaatctagagatggtttaacatatttgaagatgcatgtaggttatatgcaaataccatgccattttatataagggattcgaagcatccttggattttgggatccatgggagtcctggaaccagtcctctgtagatagttgaggtatgactgtagtctttggattgaaagactcactaatttcactcagattcaaggagaagggccatagactccaccacctgatgaaaaaatgacagtgttacattgttaagagcatgtggaatggcttctattgtgaccatctttggaaaatacagttgaccacatataccatttcatgagctgtgacaaatgtatacacctttgccagaaagttctatcagggtatagaacctacacctcttcctagttattccttccccccacaccacccctagaacctaacattgttcagattgttttcatcttagtttagcttgcgcagaactttatgtaagtagagtcatacaatacttatatgttgtaaaaagtttctttttgctcaacataatgtttttgagattcatccatattgttgaatgtttcggtggtttgtgacttcatctgttaagggacacctgggctgttttcagttccaggctgttttcagttcccagctattataaaaaggtacaatgaacatttgtgtaaaaaaagaagtctctgtgtggacatatatgtttcttttttcttggttagttgatgtgtttttctttcagcatttgtttttgatgagaagtctggttatacagtcatgtgccgcatgctgacatttcagtcaacgagatcatagttgacaatagtcatttaagattataccaccatattttcactgtaccttttctaggtttagatgtataaatacttacactttgttacagttgcttacagtattcattcagtatagtaacatgctgtacagctttataacctaggagcacccgggctataccatgtatcctagttctgtagtaagctataccatctaatctagatttgtgtaagtacgttctatgatatttgcacaatgattaccatgctgagagttgttggggttttgttttttttttctttttgaatctgtacatttatggttttccttaaatttggaagattgttgccattgtttcttctatatatatattttttaccacctttactcctctttcatcatttcctctttttctgggactccagttacacatatgttagactgtttggtattatccacaggtcattaacatcatattcattatttttcagtctttttatttccctgtgcttcagtttgctttgtcttctattttactgttttttctcctcccttgttaaataatttcagatatttcgctttatatttcaagaaattccatttgattcattttattcatgtatttttttgaattcttgagcatatttgtaatagctgtttgaaaatcaggttacttattgatttcttgaattttttcactctgaatttgttggttggttttttctcttggtcatgggttgtattttcctagtatttattttattttattttttgttttttacatatttaatgattttttaagattggatgctggacattgtaatattttgttgttgggtgtctagattttgttttcttttagagtgttttgttttgtagccacctaaataacttgctggtcagttttatcccttcagggctacattggagtttgtctagagaaggctttgctttaatggtagtagctcctacctaaggcatgatccttctgagtctttactaaatgctcccagtgtcaccgtgttctcttcactcaggttagagagaattttaggagtctcccagtcctttataagtactgggaatatttcagcttactgcccctctttcactagcttcatggagtcacttaccttaatcgtgtacagttagtactcagcagtggactctcaaatggcagatttctgaagccatttctctgccttctcagacaagcacttgtctggcacttgtcatcaattttacccacttcagctttctcagctctgatctcttgattctgaactcagtaaaactgctgtcttctcactggatttcccctccattgactgcaggttggaaattacctccaggcagacatccaagcagtggatggctcacctcctttgtttttgtcctttcatgcattatagtcctctgctgcctgttatccaaggtctgaaaattctcgtttgagaaggagcaactgtcagatactttcggatctttctctgaaattagctttctgttgtgcttgttgtgcttacctctttgtctaatgctaataccatatgaggtttttttttttttttttagaatgttttaagctatacacttctaagtgtatagcttgttgcactttcacacactgagcacccgtttagctagcattcagatcaaaaaacagcagcagctcctgcgtagccccttgtgctctgtgtcactaccccttttcttccatgtgtaaccactgtcctgacttctcatagcatagattagatttgcctgttttcatcctttaactagaatcaagcacatttagttgtgtctgacttcttttaatgtatttgtgagatttattcacattgttggttgtagtaattgtttatgatagtcgctgtatattccattttaagaatggaagtgatattccattttaagaatatgccatagtttaatctgctgttacggacatttaatcatttttggtttggaactgttaagaatattgctgctgctctaaacatttcagtgattgtcttcttgtgaatatgtttacttctgtttggtatacttaagagtgaaatttttaagtcataaggtatgcatatatttagtgtatactcagttttttaaaggcatgccgattttaattattccatttaagttaatatgttttaatatctggtagagaaaggaacattattagaaacaattgttttggttattcttacatatttagtctttgaaaaaacgtttcttaaattggcatttacttatatttgtgttaatttgggaagaaatggtatctttgttcatctatgagtcacatttggaactagctcaaaggccatgggtcctgattccttttttttttttttttttttcttgtagaggggaggtatggtgtcaagaaaacatagtcaccattattacgaaaagtaaaatatggaagagatgatccctaccatcaatcagcttacaactagaggcactgacaaatgtatacagatagctgtaatgtaaggtataacataataaatataaggagaaattaataaattattttatggtattaagaatgtataataagacatgaaggagatggtgattatgtataagagtttagataaccctgtgagatacagtgtttagaatgaggccagtttttaacatggcatgataggtgggtgggggatagaggttagtaatggatagaggaccatttccgagtgaagtctcctcagaagcaaaagcattgaagtcagaaacacaaatatccagtacattatagccaatagtcaaatgatgggactagatctggtaactggtgatgatgtcatatagtgggcagtagtgctgggatggctggggtcagggccacatggcaggggcctcaaataaaatgtgaaggagttagaaatttattctgttggcatgagaaagtgttaaagcttttgaatatagaatgatatgtgtaactttgagtttttaggaagataattctagtggcaaagttgaggataaaggcaaaagggaaaccagatgaaataatggggcactgtagtaggacagtgttggtaagtttgaggaaaaaaggagtaagagatatttttaaggaaattcatagggtttgagtataaattagaaggcaagtacaagggaataagcgtggtctttagaatcagacaaattagggtttgaatcttaaccttgcatttactaaaggactgaaagctagttccctaagagaaaaattttctcttttgtaagatgcttattctataaacatttgcaagcactggaagtataaaagcaaatgatacacatgttctcttgaagtctggtctagtctgtaagaagaggctgcactggcaggttttgaccaaattgtgtagaatctcatctgacgtgattgggagctttggctttattctggaagcaactaaaagcaatcaaaaaagactggggaataatgtgagaatgtagaaatgtaggggaatttctaaggatgaattggtagcaattggtaaacaatttgatatactgggaaagagagagactctttagaatatgactgagaatcttccaatttgagcagctggactgatagtgctaccatcattacctcacagggtttttcttagtgtgtcttgtaaatcttgcttgtccctcataaagaatggttttccttttcttcagactaaggactttttattacagtacacaacttaacaccccctgtatctctccatcacaacacttaactgtgcattgttattgcataattaattgtggtttttatttgtgcccatttccttcaccacatgagggcaagaaattatgtctagtttttctacctctgcatcttcatccacctagtaggcactcaaaatctagtagacagtttttgaattaatgattatgaagaatttgaaggctgaacaagtatcaacctacacagtggttagggcaaataaaaatgagggggagaaagtaatgaatggcaccaatttttttagtccttaaccaggacagtgagattaggcagagtgagttctttgggaaaataattgagtttgatattagatacgttgaatgtagatgttaatgagacatccaaatggatgtgtaccgttggtaatatgtagttgcaggcataccatgccagacataaggacttaaagatacagagttaggggtttgtaagcatagaggtgctatgggaatggatgacattgcgcagaaaattggtttaggttaagaaaagtggctattgacagaaacttggagagaggccatgtatgaggtaggaagatgaactagcagaagaccttattgcctttagagagatggaagaaaatgagtggatacaacattagggaagccagaggaggtgaggagtcattagcagtcttaaaagctacaaaaattaagtaggattaaggggccaaagcactgggaatatacagaaatgatggaaactttacaagaaaaatttcagtagatagaaaaatagcagctgagctagtggttttcacgtctttttggcagtaaaaggaaagaagataaagaaatggtactctgaggagctaatggagttgagggaattatgttgcaagtattatttgaaagcagtatttaaaactttttaagtcttaactgcagaatggactggaataaggagaaactaaagcaggaggacttaggaaaattgttgtagtagtcctttaattgtgaaatgtgtaaacggacaaaacttagacagttttaataagtagcattatccataaatataagacaccataaacatgatgcaaaggagaacagaagtgttgggtgttataataaaaagtgatagaaaaaaacagtttgtgaaattaagttttgaaacagctttagaaggaggtgaaggaaaaatagaaatgaataaaatgaaaattttagggaggtagatatagatcaacataggcatttattattagactatacttatttataagattattagactgagccctctgttgaagagataaagtaagagaactatgtctggtattaggattcttatggttaaattggcaagtgagtgtgcctaatatttgtagtgcttacagtttttgttattctgtatttttatagatttggtatatttcctccatgattatgtgttcctaactcataatttggaaggcagggcttcttaaaatgagatctgtgtgtcatgtgaagtatatgttaaaatgcaaattcatttctgcgttcttgggttggtctgaaaaaaattaaaaaaaaaattcatttacccctacttcatgcactgaatgaaaatctttgcgagtagagcccaagtagttgcatttttagatttccccaggtaattctattatagactgaagtttgaaagtcagtctctaaaaccttaaaagttggttaattttatttcaaaatgttttatatgcagtgtattcactttctaccatcaacctctaggacactggataggtttcttaacctttgagatttgctttctttatatgtaatgcagtacatgaaaggactttatttttgttccattgaggaatgttctgattgtatacaaacaagttaagaatttacagtgataatttaggggcttgctttaagcttcaggatttttttttaagagttaagttttgatatatattaagtggttgatttaataaattataattatcgtcgtatattgaagtcctgttttgtgccaggtaggcattatactataactttttaacgcattttctctgctacaataacctgacattggagactcaagggtcagagggtcggcccaaggctgctactctagaaagtggtagatgcccagatttgtacctaacatagccgatgcttagcatatagaatttaattaaaaacaaaattgctataggtatttgaaaggattagagggatatagtcaaatagttttcatatgtgtttagcccttttggagggaggatagagtaaataaatagaaggcactaagctttatggatgaatatgggaaatatggctttgcctgctgaaagcactcaggccacagggcctgcctacctatgctttcctcatcccggcaacagcagtaatttgtttcttgatacgtttttggatgttcataattatagaaaataaatttgcctttatgatgaaatttagtacctttttttttgaaagttcatccagttgtttatgtaaaaagaattgagtgaggtgttttatattttaggttaaaaatctgtaagagcctgattttagaattcaccagctcctcagaagtttggcgaaatatggtatgtttttgttttattttttggaattcgtattgcatgtgtatcttaatgtcataataatgacagatgtgttcagcaaatttgggcccctgccatttttaaagcattgtgtcaggtgctcactaactacacttccaagactgtatgagggacccaggtagggtgctgaaaaaatttacaagagattagtgtgctatgagaggtaaaataatatgcataaaaagattgagagggcaggtttggattttgggctatgagtattaataatagctttcatttgttgttgttgttgctgctgctgctgctgctgttgtttttgagacagagtcttgctctgttgcccaggctggagtgcagtggcaccttcttgactcactgcaacgtccatctcccaggttcaagtgattctcatgccttggcctcccaggtagctgggattacaggcgtgtgccaccacacctggctaagttttgtatttttagtagagacggggtttcaccgtgttggccaggctagtctcgaactcctggcctcaagtgatccacccgccttggcctcccaaagtgctgggattacaggcgttagccaccacgcccagccatttgtgagcacattatgtcaggcactgttctgagatacttagatgttagcttgtttatccatgacagctctatggaataggtactattatcctagttctacctataaggaaactgagactcggagaggttaagtaacttgcccaagatttctcagctagtgaatgaccaaacagagacataagtccagcctggccttggagccttttgagcaccggatctcataaaagaggggactggaagagaggacttgactctagttttcaaaatacatatagctctttaattacattgaggtcaattagtcaattaaaagatattttaactttattatttttgtgtttacaagatggaaagttagacttgtttgaatcttctaattccacataccattagtctcttgagaacccaaggggaagctgtatcatgaccacattgtttattttagggaaggtggtgtttcccatatgacctcagcaatgtctaagttcttattaggcaatgtatagttaggataggagtatctgggagattgcgttatttcttacagatactataaatcccatttattggaatcccctgcaattatttttatatattcaggccaatcctcacctaatctggattttgaagccctgcgttcatagctgcataggtatgggctcaaacattcatgttttaaccgttggtattcgcagataccttgctgttttatattgggttctgaatccacactggtaaccaccttttacgcttgactattgttgctttgaggatgttaattatgaaaggtttcttcctcctaatttcctcatctttccgtagttgatgcctcagatttggcctggataggggaatcgtgaggggaaagaactagttgcacaaaatgatcttttgatactgggatatttcagatgtatggtatctttgccagaactacagtgtcagtaaagaggcctgctaaatttgaactgtgacaatcagaaaagttcagctaaggccatgtaaggggttaaaaagatatttggttgagggaggaggatatgctttcatcttttgccatactttttcatgaaattaaatcttagtagatttatggccaagaaagagtagaatatatttttatgcagtacttaaggggttttgactgatgttttttattattattatactttaagttttagggtacatgtgcacaatgtgcaggttagttacatatgtatacatgtgccatgctggtgtgctgcacccattaactcgtcatttagcattaggtatatttcctaatgctatccctccccttccccccaccccacaacagtccccagagtgtgatgttccccttcctgtgtccatgtgttctcattgttcagttcccacctgtgagtgagaacatgcggtgactgatgttaaatgactttacgtacatacagaattagtgggactagactttgtgtttatgtgtgtgtgtttatgtgtatgtatataactattccattaagcagttgccctccttggtgactgattgctctatcagcttaggaatcttttgactgaatttgtccgtatttgaaagatctcaatctgtgttctgaatctttactcatttacatatacttaaatcttgaaacccagtatagcaaaactaagccaaaacaaacaaaaaaatccatcccttgataggaagtcagtatatctgggttttagtcagtccttcaacactagttccaaaatcttgggcaagtcagttaatcactcggaggctgcttaattttttttttttttaattcttagtatattatctgtcttgattaggtaaagccaaagttgttataagactcaaggtactattgaccaaaatgcttcagaaactataacattacataaaagtacaaggaattattacattttctcaaatgaagagatcatttaagaaaaacaaaatgctggaaaatagtccttaaaaacttttctataataattactcttctgatgaattttacattgacacctaatttgaagctttgaatattattttttgaacaatgaattgggtttatatattaaagtgttctaaccaaaggtttgtttaatttattagagttattaagcctacgctcagatcaaggtagcagctagactggtgtgacaacctgtttttaatcagtgactcaaagctgtgatcaccctgatgtcaccgaatggccacagcttgtaaaaggtaattttgaattattttacagcctttaaaaggctgtcattgtaaagtgaaatacatactgtaaaaatgaagacaatgtatagttggcaggaatactgctaagaatagtgggcctgagagttgacctacttcatttaatctccttaaaatattttagtgtctttttttctcattaaaaatatcagtagccactatcaaagacctatctgtatgtataaacaggaaagtatttctcaaaaaaacttcaaggttttaaacattttactataaacattatagaaaacattaaaaatttcttaatatgcattttaaagtatgccttttatgtggtgaacgttttaactaaacatttctctagaagtttttataacattaataaaactagtatacttttctctcctagagagttacagtggaggtaaaaggagtggcttgcaggatggagaagctgcaccagtgttattggaagtgagccaccatttgaatttgctagctcatgctgcagtattcagattagtgggtgttttgtgatcatattttctgcgattcaaatcaagacctaaaattgattctcattctgagatttacattttattatccaatgttatttggttattgtttcagttttaaaatttctgacttgttattctgacacactttaggataacctaagtcaatagtttaatacaaaaatttccctttggttatatgttttgaattaggttatatgttctgcatttacaagcacatacaggattcttacttccagcaacttggcagtaaaagaataagtagactctttaggagccaaagtcaagcttgggaactgttgctaggtggtatatatttcatagtttatgtttgtttccaagacaaggtctcactccatcgcccaggctggagtgcagtggcacgatcacagttcactgcaactttgaccttctgggctcaagtgaccctcccacctcagcctcccaagttgctgggactaaaggcacccaccaccacacccggctaagagacgaggtttcatcatgttgcccaggttgaccttgaactcctgagctcaagtggtccttatgtcttgacctcccaaagtgctgagattacagacatgaactaccacacctggcatatgtcatagttttgagagtttgaattctaacctcgggactttggtaacttatttataatattctagaaatatactgttattaggatgagtagaaattcattcagacagaaactattataaattactgcaagaaaaatgtctgctagttctgcatatattcatttttctcttgattaaaaaacaatttccaatttgagtagaacaactttgctatgtttaatagacccaggacagatttattttaaggtaataatctttcaatccttcttcttctatactggattggtttaaattacttagcttgtcttatcaatggtttttaatagcctactttaatatatgtaaaaagtagtaattaatatgttacagtttggtggcaaacattttgatgccaaagttcacatattctcttgcttagggaacagaaattgggaagatagatgtagttatcatttgataatgtatttaaactctatcccatttcaggatctcaaaactttgtaacagttacatattgtacttctaagacttagatttgtttttcttcttgtgtcttacctttttgcagtaatatttttattttcctatcatcatgtcctcattttttccctctcttctctttccctgttaattttgaaagttttgctgcctacgtgtttgaaaacttctaatctgcctctcttccctcagtgccagcaggtttattttttgttttgcaagccagctctgcctccttacagtatgacatctgatgctggagggtcgcactttcaaaaatgagtcagctggtacatggggttatcatcaatttttagctcttctgtctgggagatacaagtttggaagcaatcttggggtacttacccacaaggctggtggagaccaggtgtgtcacataggtgatttgcttgctccctgggggagagggtggagtgaaatttttgcatttgtgtcacagccaagtcactgccacctccagcatgccccagtttttagttgttatgtaattttgtgcaaaattgaaaatttaaatacattgcaaagaagatctaaaataaatatttgagctgctggagtcttttttttttttttttttccttttccattcttgtccttggaatctgaatgaattgcagtgagggtaattttgagagtctgataaagaagtgaggggttgttttttgtttttgtttttgtttttagctaagaaatttagaaaacaggtttgtgtgtgtgcgtgtgtgtgtgtgtgtgtgtgtgtgtgactgattttaagattcttgttagagttgcttaaagttggaagcctaaagtcagtgagaagttcacaaatcaggactttgtaatgccaatttgaaaatttagcttttgacctcaaaaaacattttttatctagtttgggaatttctaagtgttagaaatcaatattatattgactcattcattgttgctgatgctgtttataaaagtgaatagatagggaaatactgattcatatctcgtgaaatgaaaatgaaaggctttttagtgacttagaattttaaatatttctacatgagagagcagtagtatatttagaaataacaaagtaactggcaactgtttaaaactgaagttaattcacagctatccagtgcaaaacttcacctcaggtgatacacttttgacaggtaatacatacagtaagtgtatttttagggaaacagtttcattgttgaaccaagataatcatcattagaatgttgtatctgatttaagtgtctttaaacttaccaaggtattagattttagtttgaattgtctggagtagcggtagcggttggcacatttgttcttaaagggccagaaaataaatattttaggctttatgggccatgtggtttctgccacaagtcctcagctctgttcttttagtgtgaaagcagccatagatgatacctaaatgaatgagtatggctgcatcccaataaaacttcatttacaaaactacatggctggcctaagctttagtctgtctgcctagggagtagtttactgagccactaatctaaagtttaatactgtgagtgaataccagtgagtacctttgttaatgtggataaccaatacttggctataggaagttttttagttgtgtgttttattacacgtatttgactttgtgaataattatggcttataatggcttgtctgttggtatctatgtatagcgtttacagtttcctttaaaaaacatgcattgagttttttaatagtccaacccttaaaataaatgtgttgtatggccacctgatctgaccactttctttcatgttgacatctttaattttaaaactgttttatttagtgcttaaatcttgtttacaaaattgtcttcctaagtaatatgtctacctttttttttggaatatggaatattttgctaactgtttctcaattgcattttacagatcaggagaacctcagtctgacgacattgaagctagccgaatgtaagtgtaacttggttgagactgtggttcttattttgagttgccctagactgctttaaattacgtcacattatttggaaataatttctggttaaaagaaaggaatcatttagcagtaaatgggagataggaacatacctactttttttcctatcagataactctaaacctcggtaacagtttactaggtttctactactagatagataaatgcacacgcctaaattcttagtctttttgcttccctggtagcagttgtagggaaatagggaggttgaggaaagagttgagtttaacagtctcaacgcctaccatatttaaggcatcaagtactatgttatagatacagagatgcgtaataattagttttcaccctacagaaatttatattatactcaagagtgaaagatgcagaagcaaataatttcagtcactgaggtagaatggtatccaaaatacaatagtaacatgaaggagtactggagtacccaggtatgcaataggaatctagtgtagatggcagggaagtaagagtggccaggaaatgctaagttcagtcttgaaatgtgactgggaatcaggcagctagaagcatgagcggcttattccaggcagaagtatgagccaaagttagaagcagcatagggagtaggggacaataggcagttgaggacttttagatcataaactagctgagaggtaggaaaatgaagaagataagatgtgatagaaggtattagggagtcagcaaagaatttgctcagtaggtagatagtaagtttgtcaaacgtgtatttttaataaccttgcaagagagaaaggattgaagagaaacgtgtggtggtaaggtagttcagtcagtgagagccttgacaatgacagtggtataataggaattcaaaataaatttttctggatttaagacaaatatttaggacatgaagtaagtaagactttagtgatttattgtgaagactaaagatgtttcagataatagaggtagtaattatcaaagcaaagtttttcgaggtattaagggttggaatgaaaaatacaggtgtatctctcatacaggtaatataaaaaaagaaaaatgcagatatagtgattgaccttgaataagaggaaagatgcccatctaatgaaattagaacaaggaagtaaagatggatgtgtgatattaattgtaattgtgggtgagggtggaaaaatcaagatttttttactgaaaatattggaaccttatttaaatgccggtatatattgtgaatctgcaagaaacgactataaaatgtatttctcaaatttatgtgacccaaccatgtgacttgtcttttaagctttaaaaaattgtagtaaaatacacataatgtaaaattttccatttcaccatttttaagtatttggctcaatggtattaagtatattaatattgttgcacagtcacgatcagaactctctgttttgcagaactgaaactctgtacccattaaacaataattcctcatttccttatcttcacagcctctgacaaccattattctactttgtgtctaattttgactgctctaagtaccccctataagtgcaatcatacagtttttgcccttttatgattggcttattttacttcgcagtgtcatcaagtttcatccatctttagcatgtttcaaaatttgcatcctttttcaggctgaataatgttttattgtatgtatataccacattttgcttatccatttacctgttgatgagcgctcaggttgcttccatgttgtagctattacagataatgttgctatgaacaggagtgtacaaatatctttttgagaccctgctttgaattgttttcccagaagtggaattgttggatcatatggtaattttatttttaaaattttgagaaactgccatactcttccagagcaactgtacctatttgcgtttctaccactagtgcacaaggggtccagtttctctacatccttgccatcacatattgtgtgagagtgtgtgtgtgtgtgtacttcaatagtagtcattgtagtggatataaggtagtatgttactgtaggttagtgaccacgaaactttttatgaaacattttttaataatattttgtggaatacaaggtttgtttagaggttttgagaagactgaaaacttaggaagctactgaggggaatgggtgaagaagtggatcagaagttgagagtcttgactggctggctacctgaaatcagaaaccgtatcagagtaggagaatctcattccactgagtgactagttaagattaaagactttatcacatttgaaatttaaagacttttctccaatagccagaagattatgcatggtattttagggttggatattagcagagctgatggaacagaatcattagtggcatgtgcataagatgttaacattggagtatttgaatttatatgctgtggatgatggccatgattggaaaggttgggatgccagaccaaagtgttgagttgttttaagaaaagcctgaggtctagataacagcaagatggcagactagcactttgcagcactcatctcattgtagaaccatcaatttgaacaactgtctacacacaaaaatataagagctaagaaaccagacgagagattacctgaatgtggcacacagataagaaaagacacattgaaaagggtaagaaggacagttttacattacttgtgtcacacctccctcagtccctgagatcccagcatggagagagagactgttcacctgggggaaggaggggaagtgtacaccagacttagaccctaacattgagctcgccccagtaaaactcaatgctaggcaggcccccatggccccaaacttcaggccaatacccacaaaccaagcttggaggcccaccccagcaccaagctggatcccacagccctaggctctaagcctgtctagtgctagggtggtccctgcagccctgccctctagatcggcccctgtggcctcacatcactgcagaacaagggtccaaacccagtacttggccagccccttgtgatccaaattctaggccaacatccacctacccagccttcacactggtccttgcagatccaaggttcaggctcactctagtagaccaggatgccaggccagcagcacctgtgtaccaaggctccaggacctaccctgctgacccagactccaggccagccccggtagactcaggctgcataatctctggacaggctgactggtgaagggctttacccaataaagtcactctgcaaagactggaacaagtctcagcttcttcaaatgtgcaggtgccaacacatgactacagggatcaagaacagtcagggaaacgtgacaccagtcaaaggaacacaataaggcacctgaaccaaccataaagaaatggagatatatgaactgcctgacaaagaattcaaagtaattgtttaaggaagctcaatgaatttcaagaaaatgcagataaacaattcaacaaaatcaggaaaacagcaatcaaaatgagaaattttgagattgaaattattttttaacaaattctgaagctgaaaatgcaataaatggaatgaaaaatgcaaaagagcattaaaagcagaattgagcaagcagacaaaagaatctgaactcaaaagacaggttatttggctagactaaggaaaaaagagatgactcaagataaaaaaaataaagagttgtgttttttgatgataaaagcaacaagtctttagctagactaagaaaaaaagagatgactcaagttaaatcagaaatgaaagaggaaccattgcagctgataccagagaagtacaagaaataagaggatcataagagactgaacaattatatgccaacaaattgattaccatagaagaaatggataaattcctagacacatgcaacttgtccaagcttaatcatgaaatgaaaaatgtgaacagtccagtgacaaggattgtatcagtcataaaaatatctcccaccaaaggaagcccaagacttggtgactttactgctgaattctagcagacatttaaagaagaactagtacagattgttctcataattttatgagaaatttaagaagagggagtaatttcaaactcattttgggagatgagcattacctagataccaaagccagacaagaactctccaggaaaagaaaattacagacccgtattcttaatttagatgcagaaattctgaagaaaataataactaaattcaacagtgtattagaaggatcatttactgtgatcatatgggatttacctcaagggtacaaggattatccaacagacctgaatccataaaatgtgatatacaacattaggagaacaaaggccaaaaccatgttatcatttcaatagataattgagataattgagaaaaagcatttgacagaattcagtattctcttgtgctacaaaattttaaaaaattaagtatagaaggaatgtacctctataaaataaaggccatattgacaaacacactgctaacgttatactgaatgttgaaaagttaaaagcttttcttttgagatcaggaagaaaacgagaatgcccacttttgccatttctattcagcacagtaccggaagtccttgcaagagcaattaggcaagagaaagaaaaggcatcccagtaggaaaggaagacattgttgtccctgtttgaagatgacatgatcttgtatatagaaaaacctaaagactccaccagagaattgttagaactgataaattcagtaaagttgtaagatataaaatcaagatacaaaaatccgtagaatttctgtacattaatgacaaattatctaaaagaaaaatcaagaagacaattccatgtataataggtaccaaaaacaaaacatgcagtggctcccagtatcagtggttctgcatttgcaaattgaaccaacatttggatgaaaatatttgagaaaaaaatacaacaataaaaatactgttttcttatacaataaggaaactacagtatagcaactatttacatagcatttacgttgtattaggtactaagtaatgatttaaagtatacacgaggatgtgtgtaggttgtatgcaaatactataccattttatataatggacttgagcatccatggattttggtatctacaggagatcctggaaccaatatcctgcagataccgagggatgattgtacttagaaataactttaaccaaagagttgaaaactatgtaacattgattaaagaaattgaagatgatacaaataatggaaagttatcttgtgtttatggattggaagaattaatattgtaaaatgtttttattatccaaagcaatctgcagttttaatgcaatcctgatcaaaattccaatgttaaatttcacagaactagaaaacagtgctaaaatttatatggaaccacataagatcctaaatagccaaagctgtcatgagtgaaaagaacaaagctggagacatcacactatctgatttcaaaatatagttcaaagctacagtaattataacagcatgatagtcacatagaagtagacaggccaggcacagttgctcacacgtgtattcccacactctgggagactgaggcagaaggattacttgaaaccaggagttcgagagcagcctgggcaacatagtgagaccctgtctctaaaattaaaaaaaaaaaaaatttttaaagccaggcgcggtgctgtgtatctgtagtcctagctacccgagaggctgaagaaggaggattgcttgagctcaggagcttaaggtggcagtgaactgtgattgcatcactgcactccagcctggatgacagcgtgagaccctgtcttttaaaaacaaaagaaacaaaaaaacagacacatcgacccacaaaacagaatggagagcacagaaataaattcgtgtactatcagttgattttggacagggtgcaaagaacacagcggggaaagggaagtctgttcaataggtggtgttgggaaaatggaatatctacatgcatgagaatgaaatcagacctgtatctcacatcatataccaaattcaagttaaaatggattaaggtcttaaatataagacctgaaactgtagaactaccagaataaagtataggagaaatgtacatgacattggtttggacagtgatttttttggatatgaccccctcgaagcacaggcaacaaaaataaaaattgatgaatgagatcacatcaaactaaaaagcttctgcatggcaaaaagaaaaaatctacagaatgaaaaaactcacagaatggaagatagtatttccaaaccatatatctggtaaggggttaatatgcaaaatatataaggaactcagacatctcaatagtaagaaagcacctcacttaagaaatgggcaaaggacttgaatagacatttctcaaaataagacatacaaatggccattagaatggctattaaagtatgatgaggatttgcagacaagagatatattgcgcatggcaggaatgtaaattagtacagcatttatggaaaatagcatgacgtttgctcagaaattaaaaaatagaactaccatatgattcagcagtcacacttggatatatatgtatttttatatatatatatatatatatatatatatatatatatatatataaataaaagaataaaatcagtgcgctgaagagatctgtactcccatgttcctttcggtatcatttgcaatacccatgatacagcatcagcctaagtgttcatggataaagaaaatgtggtatgtatacacagtggaatatacttcagccataaaaaggaggaaatgctgtcatttgtgacagcagggatgaacctggagggcattgttgaaagtaagccagccacagaaaggcaaatactacatctctcttaaatgtggaatcttaactcagggatgtaaaatggtggttaccaggggctgggggtgagtggactgggttgcagaggtgttggtcagaggacacaaaatttcaattaagaggaatttgttcaaggtatctattgtacaacatggaaactagttaataacagtgtatttttggaaatcactgagagtagattttaaatgttctgccataaaatataagtatgtgaggtattgcatgtattaattagcttgatctagccattccacagtgtatacatattttggaacatcatgtgaatacaataaatatgtatccttttttttgttttttgtgttttttttttttttttttttttttgagacagagtcttgctttgtcacccaggctggagtgtagtagcatgatcttggccactgcatcctctgcttcctgggttcaaatgattctcctgcctcagcctcccaaatacgtatcatttttatttgtcaccaaacctggtgtcttgacaggtgaaaggagatatgagtatgagtgaaactcttacaaagatagctgagatgatcattgtcagaggttttagtatgtctaacaaagacaagggagtgatttgctgaaatgaggaggtgaagatgtctcaaatttagaaaggcaaatattttgggggcctagagtattgggatagaaccatgtacagggacattgaagtcactttgctgatgatgtaactgggggcaaagtggaggcttaaatatgtgggtttaattgaccagaacagggagggaaagagaaggcattcggttagatgacatgagtttcaaattacaagctgtttttttgttgtttttaatacagggaattttgccgtttatttttaatacagggaattagaagaataatggtggacttaggagttttaggagtcacataaaagggttgcatgataggcagtgcctttggggaggagccaggctttcatgaaagctaagaagttgaggggccattatgtaaagtagttgaagatgggggatagcctatgggtcttcagaaagcctctgggtttctagagatctttctggaagaggttaggaggatgagtcaagtttagaatggaagaggatagatgaagcttgcatttggtagtaaaaaccaaggatgaatgtgtgaggcatgattatttgtcacaagtttccaggtgacactctggcactcagtccttgttgaattaagcaagctttcttactgttttacttggctgtgttaaagtagagtaacaggaaattagacttcccataatgtctcaatagagctgttttaagtttctagccataattggtatgggaatatagctctaaaccttcaaggctgctaccatttctcacattcctatatctagaaactgtagccaggcagcactttcagtgaggtgagtgggtccaggtttgttcaattacttttgtaccaacctaataccttaatagtatttgtcctaatttacaaaaataggtaatgaaggattcttggttttacctcagagctaactgtccattaatggcaacatcccacatttgttgaagcatgattttgttgtttgtactttgacatgcatttgatattttaagtaacgccttgaatgcatcttaaataataatttatatacagttaacactcctgtttgtttttaggaattcttagtgttgccaatttcctaggatgtagaagagcatagtaaaaaaaaaaattattaatactcttaaacaagtgtttttaacaactggacaccattttgtagttaagcctagaaacagaactaatgcaataacccagatcttttcatttattcgtatgtttattttacctggatttactttagttttttgttttgtttgcttgttgtttgagatattgtcttgctctgtcataagtgatacagctagagtgcagtggtgtgatcagagctcactacaactttgaattcctgggctcaagtgatcctcatgcctcagcctcctgagtagctgggactacaggcatgtgccaccatgccaggctaaaacagtgtttttgtttgtttgtttgttttttgtagagacagggtctcactatgttgcccaggctggtcttgaactcttggccccaaagtaatccttctgccttggcctcacaaagtgctgggactacagacgtgagccaccgtgcctggccatggattttgtttaaatttataaataccgtgttgtagaatctctttgtatgtattaatttagacaccttagttttcttaatgatttcataccagtttctcagtaacttcagattttttttttgagatggaatcttgctctgtcacccaggctggagtgcagtggcgcaatttggctcactgcaacccctgcctcccaggatgaagcaattctcctgcctcagccacccgagtagctgagattacaggcatgtgccaccatgcctggctaattttgtatttttagtagagacagagtttcaccatattggccaggctggtcttgaactcctgacctcgtgatctgcctgcctcggccttccagagcgctgggattacaggcttgagccaccgcacccaaccagatcagattttttatgtttggtatttactctttacctctaacactaaggctatgtttccagatgtataactataaatggagcaggggtaaatacaacaagtgatttatggttcagtaaagcaagttagttttaataaatgctcagtttactgtaaatgtcagaaatgccccaggactctgtctattgacaactcttctatttccatcgtcagctcttgccctctgaactgtagactgatttatccaacagttaagtctggttctcaaacttaacctgtcctaagtggagctcctgattttcatcatcccaaacctacgtctctcataatattttgccatctcagtcaataacagcttttttcccctcctgtgcagccagaagccttggaagcatcttttaatttctctatgtcacatactcaaattattggtaaatcttgttggctacaccttccaaatacccataattcattttattttttttaaccattatacttctgtttactctgtttaaaggcatcatcttttggcaaaagaaagaaattgtttcccaggagtcttgtgctttacagtgtattctctagtagcagccggaatgatctttttaaaatctgagttaaaccatgttatttactatgcacagaacttttcagaggcttcccatcttaaaagtaaaatctggagatttacatggccctgtgtgaactggcttcctttcatttctgtgatctctctaccgcacttgctcatctctgtctaaaatggctgtatctttgtcatcttaaggcttgtaactattgtctgtgaccctgtgacccttctctgtgataatccatgtggcttccttcctcacttgattcagagagctctctgctcaaatgatgcttttcctgaccggctgcatcaaagaatgatcatgttcttctgcattccccttaacttgctgcatttttcttcatgggatttaatgtcatttgacttcttgcatattgatttctttgttgttttgcttctttaccactagaatggaagcctaatgacagtgcctggcatacagtaggtagtcagtaactacttcctacatgaaagaatgaagctagtaaatttatgcaaaatgtttgtttctgcctttttaattatcttgcagaaatgtaggaagaattttttaggatcagtttatgtatatatataaactgataagtatgtacacagcctgtagaatgtgggtaataccacctatcatctttttgagcatttaccatgtagcaagtgactgtgctccataatccacatgcattatcttgaatttgataggagagctgagctttttaatcagagtagaccatagaaatatagcttggatgcttttatttcctatatgacttcttcggtgtacttacacatctataagcatttatgggtcaagtaacatactgaaatattttctgaagtaaaagaaatctctttaatcaaataagtggttctcctataggttctcatatcatattcttggtctccagattgagaaggtccttttgtatgcttccagtatactcgaaaccaggaggtagtctgacatctatttcccttcaaggtcaaataattagagaaaggtttaattgacactgttatcgagacagtatcagaagagaaattgaaaaatctctgttgagactctgtaatttgctaggttctgaggatacgacagtaaataagttattaggtcatatacctcagcttcccatttaattaataggcagttattatatataatagaataggtactggttggtatgagactacagaggaagaatacctaattatcttaaaagtggtatataagctgaaaccttgaggatgcttgggaatagctaagaatcatagtgttctaggcaggaagaataacacctggaatcagagaagacagcagaacaagtccactgagtagtaagaagttccgtattgcagtaatgtagagttagctgatgggagaaggtgtgaaaaaatgcatttggatatagaaagccaggagcacgctaaggccctttgctttcttctgacagacacaggagcccatgaaaggcatcacgtgtggaaggaagtgacacgagtcatagtctgccctgcttttctaccacctgaagcatcatataaacctatatcttctgtttcaaatgtaaggaaacatacttctagttcacagtagggatagttgaatggatgaactgtttctggctgaatcttctcaaagtagaatgagcaaaacatggctatattatgagattatctaaggaaagatttttataatcatgtgaattttgaatttctttatatcttaattattccaagagatttttgggaacattaatgagaatagtttacctttagtacatagaacttcttgtcagctataaatgaggatcaaacccaaaagagattaacaaaaataaaagggatgctatttccccaaattagcagaactttttgattcaccgagttctttgttttttaactgctgtctgcctcttaacataattgagtattcagtgatataatataaataaatcacttactaaatgctgatgaacgcttgtttttactaacatctcagcttgtagcatatcttgttgcccataccagaaaccctacccattacctctggctcttccagcttatttacctcttacaaactacttattttaaatatttcctgggtcaatttcatctttttttttcttcctactgctacattatttgtccggactattacagtagtcattacctaactggattgcttgcctccagttttgcccactttggtttcagttattgaaaaggcaaattttgtcagtcacttaacctgcttaaaacccttcagtgactccttgtgttttagttcttatctaatggtttatgaatgtagtgggttacgaatcttccggaagagtgcatttgcactcctgaagtgtgcatgtatttttatgaggagaagatctttagcttttattagattactcctcttttggctaaaaaaagaattacttatcatggctattgaggtcttccatgatctggcttttccctgcctctccattttcatatttttgaaacttttcttgaactttaggctcttgaagtgcttggaattgtcttataatatcatgttttcaggcccctgtgcttttactcattttcctctatctgaaacattcttcccctgtctgaaacattcttcccacccatagtgtcatcagctcctagtcatcttttgtgattcacctctcctaagttgcctctctttttattgctattcctctgtagtcatctctgtgatattgaaatcgtggctttctctttctttagacatcataattccttgaagagccctttttatgtagcctgacaagcagaatttatactgaagtagacatttggaagtcatttagttattgctcaatccttgaaagatgaaactgtccaaagggaaatttaaaagggaaagtatctaatagctgatccctggagaatacaaatgtctctagatttggaagggaagtctctgaaaaacagccagagagaagggaggaaaccaagaaatagtgtcctagacccaaaagtcaataatttccaaaaggtctatatttcttaggaaggccaagtaaaataagtagtgaaaagtgatgacctgactagagcagattcttcatttccagttacagaagctaggttataggtacagtaagctctggacatgttcttttcccacatggaacgttattcgtgttctccactgctgctttgaactattccttttactctctcagcataaaaatccctgctcctttgaagcttatgccttctggttttgtcttcctctgtctgccttcagtgagctatcagtttcttctcggtttttgaggactgctacctgaacttattcttcttcactctttcccaaatctgctgccatctttatttatatgattaacccctcagtccctagcaagtcaaattcccatccgtttttatctacagtaatgttcacctctgcatcactgctgcttcccatgtctacaggcatgccaactcaaaactgttttaccccaaaagcttcagtgaaaacaccttacctcaggttgccgtctttgaatgcttttctactatcttaaacttgttttctttcattgcgccttttcttccttctcttagagacttgtcctatggctcttctactgtcttccagctacaattccctcttgttcctgacatgtactgctcagagccaggagatggataagtagtagtatttcacatgcacttttttctctggggagcagtagtcatacaataattgattaagataaaacaggtttttatcagtttatataagaggtaaagaaaacaatggggtagagttttaaaaattgtcaaaaacaacacattttaccatattaatgatttgaagtgtacggtacaattgtggtaactgtgcacattgttgtgcagcagatctctagaactttttcatcttgtaaaacagaaactctacattcattgaacaactcttcttttcccccttcttcctattccttggcaaatgccattctactttttgcctctaaaatttgactgttttggctacttcatgtcagtggaatcattaagtatttgtcttttgttgactggcctatttcacatagcaatgtcctcaaggttcatttgtgttgtagcatattacagaatttctttcttttcttttaggctgaataacattttgttatatatgtataccacattttctttaaccatttgttgatggatgtttaggttccttccacttcttgaatattgtaatgttgaaataaacatgggtatacaaatatttttttgagaacctgtttttaatatatttggaagtatacccaggtgtaggattactggatcatatgtattctatttttttttagattttttttttttttacaggagttctaggttcacagaattgagaggaaggtacagagatttcccctgtacctccttccacacacatgtatggattctctcatagccagtgtgcatctcccaccagagtggtacatgtctttctgatgaacctatattgatacatcataattacccaaagtccgtagtttatattagggatcattcttagtgctgtacgttctgtggctttgaacaaagtacaatgacatgtatctaccattatatcatacagaatgttttcactgccccaaaatcctctgtgttctgcctatttatctttccttccctcccactccttggcaatcaccgatccttttactgtgtctctaactttgccttttccagcatattatatacttgtaatcatacagtatatagccctttcagcttgactatttttacttactaatatgcattaaagtttcttccatgtcttttcataggttgttagtacctctctttttccattttctggacatatcacagtttgtgaatgaatattcacctaatgaaggacattgtggttgcttccacattttgctaattatgaataaaactgctgtagatatcggtgtgcaggtttttgtgtggacttaggttttcagcgactttctgtaaatactagggagtgcaattgctggatcttatggtaaaagtgtttagttttgtaggaaacatccaaactgtcttctaatatgacgctatccttttacatttttcaccagcaatgagtgagtgtttcttttgctctacatcttcaccagtatttggtgttatcagtgttctgaattttgtccattctgataggtgtgtagaggtatcttataattttaatttgtgtttctctgatgccatatgatgtgtgggacatcttttcatatgctgaattgccatctgtatatctttggtgaggtgtctgttaaggttgttggccaattttttaattgggttgtttgttttcttgtggaattttaagagttccttacactgtctctgtctgtcttgtctgtctgtctgtctgtctgttgagacagagcctcgctctgtcacccaggttgcagtacagtggcacagtcttggctccctgcaacctctgccacctgggttcaagtgattctcctgcgtcagcctcccgagtagctgggattatacccgtgcaccaccacacctggctaatttttgtatttttagtagacacggggtttcactatgttagccaggctggtctcgacctcctgacctcaggtgatccacccaccttggcctcccaaagtgctgggatgtgagccaccacacccagcctccttacacattttagttaatagttctttatcagatgtgtcttatgtaaatatttcctccctatctgtggcttgtcttttcatactcttgacattgtcttttgcaaaagacaacaatttttaagaaacaagtagacaattttaatgaagtctggcttatcagttcttttcttcatggttggtacctttggtgccgtatctaaaagtcatcaccaaacccaagatctagattttttcttatgttatcctctaggagttttatagttttgagttttacatttaattctctggtttattttgagtttatttttgtgaagggtgtaagacctgtgtctagactctttttttttttgcatgtggatgttcagttattaccactgtttattgaaaaagactattttcttcattgtattgcctttgcctctttgtcaaatactgccattgctcctttgactatgtgggtccatttctgggctctctattctgtttcgttggtctgttttgtcagttcttttgtcagtaccacaccatcttgactactgtagctttacagtaaatcttgaaatcagtagtgtccatctgactttttctgtcaatattgagttgtctctcacgggtcatggaatagctcttcattatttagttatttgatttctttcatcagagttctaaaacatttttcctcatatatcttgtactaattttgttagatctgtacttaaatatttcattttgggacacgctaatgtaaaatggtattgtgtttttaatttcaaattcaacttatgcattgctggtttacaggaaagctattaacttttttatatgaaccttgtatcctgcagctttgctataatcacttattagttctaggaggtttttgttgtttttctttttctttttttcaattcctgtctatgttctacacatatagtcatttcatctgcaaacaaaggcagttgtatttttttgttccccgttagtataccctttatttccttttcttgtcttagctaggacttccagtatggagtggtgaggtggggatattcttttcttgttcctgatcttagtaagaaagtttctagtttctcatcattaaggatggtgttagctgtatgttttttgtaaacgttctttatcaagttgaaggtgttccctctattcctagtttactagtttctatcatgagtgggtgttaaattttgtcacatacttctctccatctatggtatggtcatgtgatttttcttctttagtctgttgatatggtgaattgcattaattgattttccaatattgaaccagtcttgcatacctgggataaatcctactttgtgctggtgtatacttctttttatacattgtagtaaaatttagtttgctaatactttgctaatacaaagatttttgcatctttgttcatgacaaatatgggtctgtagttttcttttctgataatgtctttgtctggtttgggtattagagtaatgctgacttcatacaatgagataggagcatctcctttgcttctgtcttctgaaggagattgtagagaactggtgtggtttcttccttaaatgtttagtagaattcatcagagaatccatctgggcctgatgccttctcttttagaacattattaattatggatttaatttctttaatatatgctttttaatcaagattatttcttcttagagttctaatagattgtgtcttttaaggaattgattcatttcatctaggttaccaaattcgtggccgtaaagttgttcataatatcccttgattatccttttaatgtccatgagatctgtaatgatgtccactcttttcattctgatattagttattttgtcctttgtctctttttcttagcctggctagaggcttattgattttattgatcttttcagagaactagttgatttctttgatttttctctattgatatcttgttttcaatttcattgatttctgctctaatttttattacttctatttggatttaatttgctttttctagtttcataagttggtagctcagataattgatttttagatcattcttttcttttcttttttttttgagatggagtctcgctttgtcgcccaggctggagtgcagtggctcgatctcggctcaccgcaagctccgcctcccgggttcacgccattctcttgcctcagcctcccaagtagctgggactacaggcgcctgccaccacgtgtggctaatttttttgtatttttagtagagacggggtttcactgtgttagccaggatggtcttgatctcctgaccatgtgatccacccgcctcagcctcccaaagtgctgggattacaggcgtgagccacagcgcccggcctagataattcttttctaataaatgcatcagtgttgtatgtttctttctaagcgctgctttcactgtatcccacaaatttggataagttgtgttttcatgatattttcaatttataatggtattgggacacaacccaataagtaagtttaggagcatttgtatgcataagcacaattgttgacattattttgaacaaattcttatttgttagatcaattaagtataagaaaaataaaaggttttattttactttcagtgtttccttcttttgatgctctatatatgtagatttcagtttctgaacttcccaacacttattgcaaggcaggtgtactgacaacaaatttccttaatttttgtctgagaaagtgtttctttttaacttttgaataataatctcacaaggtagagtgctaggtttatggtttttttttctctcaacactatgaatatttcactctttttcttgttggaagtcacatgtattttttcttttttttctttttttcctctacaggtaaggtgattttttccctctggcttacttcggatttttttcttatccttgaatttctgtaattacaacgttttatgcccagatgtagttttttggcatattttcaagctcagagattcttcatccatgcccagcctactaataagcccatcaaaggcattcttcatttctcttacagtgttctgttttttgtttttgtttgtgttttatctctagcacttccttttggttcttaggattttcatctctacttttttaacatgttgtttactcagttcgttagggcccttagcatattaatcatagttgttttagattccaagtttgataattccaaatccctgctgtatctggtcctggtgcttgttttttaccttttattatgccctgtaatttttttcttgatagcttggacatgatgtactaggtaaaaggaactgttgtaaatgagcatttagtaatgtggttgtaaggtgtagggagaggagaagagttctgtagtcttatgattaggtcagtctttttagtgagcttatgcctctggactgtgaacttcacaagtgtttctcaatcccacccctccctcattcccttagttagaacaagatagctataatggcctgaagttgggttatttctcttcccccacatcattttggctttgctacaaccccagcaggttaggctctggttaactagtttctcctgaggacaaaccttgttaagactggaatagtctagtgtacttgaaaatcatttctctttgcctcccctgctgagggttgaggatttttctcctatatttactgtgagaagctggtcaattgcctggatgtaaaacttagaaaattgtagggctccccctataattgggtaactctggagtttttagctttcagagttgtccacactgagccttcatcaatttgtctattacacttcaggatttcctaccccgtggaggtttctgcttgtgtttttcatctcagataaatggtgatgctatgtgttcacctgttctctgctctcacaggcagtcatttgccctgtgacctcacttatcctatctcagatttgtttatttttaagtttgtcattttttacttgttgttaggatggagtggtgacttccaagctcttcatatgtggaactggaggtaattctatttcaaattttttgagggaccttcatgttgttttccacagtgcctggcaccattttacattttcaccagtggtgcataagggttccagtttctccatatcttcaccaatacttattttctgtttttttaatggtggccatcctaatggtttcagatgatgtatcattgcagttttaattgacatttctccctactgatcagtgatggtgagaatcttttcatgggcttattgcccatttgtatatattctttggaaaaatgactattcaagtcctttgtccactttttaactagattttttgttgttgaattgtaggagttatttatatattctggatattaaccaaataacagatacatggtatagaaatattttctcctgttttgtaggttgccattttactccatttactgtttgctttgtagaaatttttgagtttatgtatccccagtttgtctatttttgctttggttgcttgtgcttttggtgatctattcaagaaatcattgccaagtccaatgtcatgaagctttttctccatgttttcttgtagtagttttataattcgatgttttaagtttaggtctgtaatccagcttgagttcatctttgtatatggtgtgagataaggctccaacttcattcttttgcatgttagattccagatttcccaatacagtttgttgaagaacctcttttccctatttctgtgttctctcttctgctcacttggtctgtatatctgtctttatgccagcaccatagtgttttgattactatagctttgcaatatctttagaaatcagggaatgtgaggcctctagctttgttttttgtccagattgttacggctattcagggtccttcaagattctgtattaattttaagattttttttttctgtttctgtagaaaatgccattgggattttgatagggattgctttgaatttgcagattgctttgggtagtattgccatcttaacagtactaaatcttccaattcataaacttggatgtctttccatttatttgtattgtctttaatttctttcagcattgttgtataattttctttttcttttttttttttttttgagacagagtctcactctgtcaccaggctggagtagagtggcgtgatcttggctcactgcacctctgcctcccaaattcaagtgacaactcctgcctcagcctcccgagtggctgggattacaggcgcgcgccaccaagcccagctaatttattttgtatttttagtagagacagggtttcaccatgttggccaggatggtctcaatctcttgacctcgtgatcagcccaccttggcctcccaaactgctggggttacaggcgtgagccaccacgtccggcctatgttgtttaattttcaatgtacatgtctttcatttgcttggtgaaaatattccctaagtattttttactctttgatttcattttaaatgggtttaaaaaattaatttcctttttggattcttcatttttagtatttagaaatgcaactcttttcttaatattggtttttatatcctgtaactttactgaattcctttattctaacggttttttttctttttggtaggctagagttttcaacataaaagatcatgtcatttgctaacagaagtaattttacttcttacttttctcatttaatacctttcatttctttttcttgcccagttgctaactctggcaggatgtctaacagtatgttgaatagaagtggtgaatgtgtgcattcttgttccatttgtgatcttagagggaaaactttcagtttttcatcattgaataacattagctgtggtctttctatatatggccttcattatgttgaagtaatttccatatctttctagcgaattgagcgtttttgtttttgtttttttgagacagaatcttgctcttgtcacccaggctggagtgcaatgatgctatctcacctcactgccagctccgcctcccgggttctcaagtgattgtcctgcctcagcctcccgagtagctggaattacaggcgcccaccaccatgcccgattagtttttgtatttttagtagagacagggtttcactgtgttggccaggctggtctcacactcctgacctcaggtgatccacccaccttgcccttctaaagtgctgggattacaggcatgagccaccctgtctggcctttttttaaatcctaaaagggtgttgatttttgtcaaatgcttcttctgagtcagttgagattattggtttttgtcctaagaatgcagtgtattaaagtgatttttttgtatgttgaaccatccttggattttatgaataaattccagttgagaatttatgaaatataatctttttaatgcattgttgaattctgtttcttggtattttgttgattttttttgcatcagtatttattagggattcttttttcttgtaaatttgtctgatttcgatatctaagtaatgcttacctcataaaatgattttgaaagtgttctgtcctctagttttttcaaagagtttgagaagggttgttagtttttaaatgtttggtagagttttcagtgaagccctggcgttgttggacagttactgattcaatctctaatagttataggtccattcagaagttgtatgtatttgtggctcagtcttggtgggtttgatgtgtctatccgttctcaaaggttatcaaatttttttgggtcatagttgttcatattagtcttttaaggtcctatttatttctgtggcagcagttgttgtatcgcctttctaatttccagttgttgctgtttgtgtctattctcttcatttcttaatctaagaatttgccagttttgttgatcttttcaataaaacagctactcctaattttgttgattttttttctattgcaaaaattttctatttttctctattttattttagctgtaatttatttttattttttttattttttggtaagtttgagtctcatttgttcttcatttcctaattccataagatgtaaagttaatttgttgatttaaggtttttcttcctttctaactgtgaaatatgacactgtaaaacagattttcattttacagatgaattatgtaaatatttagttctacaattttacttttttttttatcatatatcacgcattctggatattgagactataaaaatgaaaaaaacagttcttttcttttcttttttttttttgagacagagtctcgctcttttgcccaggccacagtgcagtggctctgtctcggctcactgcaagctccgcctcccgggttcatgccattcttctgcatcagcctcccaagtagctgggactataggcgcccaccacggcgcccagctaatttttttttgtatttttagtagggacgtagtttcaccatgttagccaggatggtctcgatctcctgacctcatgatccgcctgcctcggcctcccaaagtgctgggattagaggcgtggcgtgagccactgcacccggccataaaacagtaattgaaggttaacctatatttcaaacaactgaaaatactgatttgttcatatttattttttcatgaaaatcgtcttgttctatatttaataggtttttgttcggttcatggtactaatgagttcacaattgtaggtttagtcttgaaataatctaaattcacttcatcccattgtaacagaccctacatcatactgaatatagttgtgtcacagatgtctcttacagtctatttctacaaggcctagaactatttaggataccgttgctcactcttacaaattttaacattaaaaataactttgagatggaatggagttttgcaacgtctcagaacaaagagactccacttacgagaaattactaagcagtggttcctagctttatagactaattatataagccttcagttgtttctattttatatctgttttttagattcctcacatgttcattctgctttaccttctgtataatataaaggtcactctacttacagtcacattttccttttaatctttccgtttttattttttattctttgctttgttcaccattagtttttatattgcatctcacagttcatgtttcttttttgtcatttctgattctaattgtaattctaatcttacttgtttggcgttcactgtcacatcttcactttctggcagctgtttctatggcttcatcttcttagtgcttgtattcattgcttatattccagttgtcttgtaaattcatttttttagtgaactaagccagacacggaaagacagatatcacatgttctcactgatatgtgggaactaaaaaaacattgaacacaaattttacagctgacacacatatgcctgccacctagattctgccattaatacttacattgctttatcgtataaccatccttgtatccatcaatccatcttaatttttatgcctttcaaagttaattgctaacattagtacacatccctctaggtgaaaccatttttaaactgattgctgtcaagttgtctagagtaaactctgatcttccaagtatgacttattccataggacttctgtttgtagcactgtcccattaattcaatcatagaaataaataattggaaagtattgtctgttttaaatggataagtattttcatgtgtctgactttagaggacttttactagaaggtctcacttgaccttcattgctgactaaataagaaaagtttgttttattttaaaaatcaacacattataacatactttgttttttgagtaataagaaaagtcaatggaatgtgaagaaattatagcaagtaagctgttgttcctcttctaggaattttgattatgaacagctgataaggtattactaagggatctcatcagggaaattcctttctttccttctttttcttttgcatcctctccctttcctcactttcttttcccctttttcccttttcccctctcctcttcttttctttaatagtacttggggaaaggtataccttttattttacggatttgctcagggtaaagagagagttaagaggccaaatgattagatggcagggtcactggagggcagagataaataatagatcctaaactaagggttggcaaacattttctgtaaagggcaggtggtatttgttttaggttttgcaggcctaaagtactcaactctgccattgtagtatgaaaaacagccataaactatatagtatattgtattagtcctttttcacgctgctgataaagacgtacctgagactgggaaaaaaaaaagaagtttaattggacttacacttccacatggctgggaaggcttcagaatcatggcgaaaggtgaaagacacttcttgcatggcagtgacaagagaaaatgagaaggatgcaaaagcggaaacccctgataaaaccatcagatctcgtgagacttactaccacaagaacagcatgtgggaaactgcccccatgattcagattatctcccagggggtcactcctacaacacatgagaattatgggagtacaattcaagatgagatttgggtggggacacagagccaaagcattatcatatatagtatatttataaatcaatgaacttgggtatgttccagtacacttgatggatactggaacttgaatttcttataattttcacgttacaaactattattatttttcaaactttttccagccatttaaaaatgtaaaatattctaagcttgagagacataaacaggtggtggactcgatttgacttacgagctatgatttggtgacccatatggtacaaggatttcccttgcgcaggaactataccaaaaagtcgttattagtgtatacttttccaacgtagaggcccttttatgtgctctaaattttgtatgccttttacatatcctaaattagatagtgatgcagatgaggtcagataaattaaggtgtcattggagtgaggaaaaaagaagaatgcttaatatagttgattccaccattaacttggaggttctggcatggcttcctttagtcctgtctcatcagtgtagctgctatttgttaaacttggcacagaaaggcagatcaaatatagagggctactttcagatcctcctcttaatagttattaaaagctcaagtttagtagctgagttcaaatctcttattagatgtatgacttatgtaatttctctgagcatcagcagaatgaaggtttatagtgctaactcatacacttgtgagggtgatagtaacaattacaacaataatagtagtagctaacactttcatagtgcttagtatgtgccaggcaatgttctaagcactttatatatattaacacatttaattttctcacaattggtaatgtgaagaagaataatactctcaccatgataatgctcgtagatactaccatttaaattacgaggaaacttacgtccagaaagatcaagtaattcattcagggtcacatagttaattaagaggtagagccattatgtgaacttgggcatctggcctcagaatgcgagctcttaactgttaaggtttattcccctctaaagtgggaatgtctataaaatgcatagcacataggtcctcaacaaattagagagaaattaactttgcagaatatagtggttccatttcctcaagaaacttggtctgcctttgtggtaccagatagatgcataaatgtgaggttcacgttatgattcttcagatttttttcccagttgcacattagttatgagatttttttccatagaattcttcagtattactcatgttaaaaaataattattttgaaatgaatcaccttctgcaaaacaatatagtcaaaatgacttagactctgaagctgaaagactcctggactcctattcttgtcttggttctgtactttctttgagatcttggtttcgtgatttgactccagttctatttctctctttggctgtggactgaagataatcctcaatgtcatggtattcaaatgagataacccatgtcaaagtgccccgcagtgtgtgtaataagttatcaggcatccagcaaatactagtttcttgctcacctccctattacataaaataactttaaaattagaactgaagggactcataaagctgggaaaactgaggcccagacaattgtttccctaggatcctacagctgcttagggacagaaggatgacacttaatgcagatcttctgatttttcagtctcgttctgtttgtgctgatcactctcctcctatgtagtatataaagaagtactatgtatctgacaagttttggggaggttattttagtataaaagctcacattaaaaatatctattttggtattttgacttttgtaatttttttaaaaatcagagacaatttagtaaatctcggggccattcaaatcatgtatatgaagttttataactgctattgttttccccttatagttattaatttttaatatgcaagtagcatccttactatggaaagaataaataagaaatcaaaagatggaagaagttttaaaactcacttaatcacatcatccagagaaaatgctattaatgctttgaaataacaatgggttcatcctacacctgctactttttaactgacttttgtcagttagtgatatatcaggaagactaggcagttctttgtcgaattcaaatcttgatagtttttattttattgtgtgacctattatcagttcatttaagtaatcttttattagacatgtttttttcagatatagaaacaatgcatttttgtaaaattgtcaaattattttccacaagtaggatctcattcacacggtattcgtgtttttaagactttcgatacatgttcactgttttcctccagaaaggaaatacctacttacattttcaccagggaagtcactgtagtttgggagaacatgacattatctccctttctttactgaagggtagcaatatatattttaaattttctagattctgcatttccattttatttttacatgttttcactttcatattttgcatagtgctaaatctgtgataacagtttttaattttcaggtggggtttatagcttcatagatagcaaaatgtctttaagtcatgtttatgcaatttttacatgtgctttgtgggttttcttttttctctgtttgcatgcagtgagtggtaaatttgcatggaacatttgaaaaataagcaccagaatgaattgaatgtgtgtgaattatcactttttgtcacctacctggacagagagtgaaagttcttctgttgagacaaaattggatctcgtgtgtttcttcacttacttgctgccttgttaattgttgagtttctcatgattagcaagatgtaaactctggcattggtcatctagtcctacatgttagataattctgtgaatgttttgaaaatcagctgaactatttttcttataattttgattctttgtagttgttatttccatgcttgtgaatttgggaccaaaaggaatggctatgacttatgtttctttggatccagtagtttattctactgaaattatggtaattttatcagttagtagaagcatagactctttagtgttgaatataatgtcatctaacttcttacatggtcacataattcctccctcagttttggtagctctcctgaaacagccataataggacattaatctgtgtactttaagttgacagagtaaaaggaatataagatttcacgtgatggaacttaaaatattccatcttgtgacgtggaactaatgaggaggtttagtgacaagttttagatttaagattctacactttggagcctctttttctttttctttttggtttttgtttgtttgtttgtttttgagacaaagtctagctctgttgcccaggctggagtgcagtggtgcaatttcagcttatggcagccttgactctcaggctcaagtgatcctcagcctcccaagtagctgggactacaggtgcatgccaccacacttggctaattttttttaaaaaagtttgtagagagtagcctcactatgttgcccaggctggtgttgacctcctgggctcaagtagtcggcctgcctcagcctcccacagttatgggattgattacagtcatgatctgcctcactcaggctgcagccgctttctttcagtgtaacactatctttggatattccacttacaaatttatttttaagtctcccatgttgttgataattgggaaaaatagtttttattccagatagatattctgtgttaactataagtcaaatgtttacaagctgttaaaaatgaaatactgattatgtaaaagaaaaccggattgatgctttaaatagactcattttcctaatgctaatttttaaaatgatagaatcctacaactcttagctgtaaaccttgtgatttttcagctgttgtactaaacaacttaagcacatataccatcagacaagcccccctccccccttttaaaccaaaggaatgtatactctgttaatacagtcagtaagcattgacattctttatcataatatcctagaaaatatttattaactatttcactagtcaggagttgtggtaaatagtgcatctccattttctacttctcatcttcatacacaggttaatcacttcagtgcttgactaacttttgccttgatgatatgttgagctttgtacttgagagctgtactaatcactgtgcttattgtttgaatgtttggtacaggaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaaggtaagatgttttattttcaattgagaattgttgcctgaaaaccatgtgggagatttaaatgtattagtttttatttgttttttcttctgtgacataaagacattttgatatcgtagaaccaattttttattgtggtaacggacaggaataataactacattttacaggtctaatcattgctaattagaagcagatcatatgccaaaagttcatttgttaatagattgatttgaactttttaaaattcttaggaaaaatgtattaagtggtagtgaatctccaaaactaggccacaacatctatttaagttatagaataactggactaagtatctggcatgagttctggaacctcaggatgatgtgatgaaagaagcagaaagaggaaacagtgggttttttccatttttgaaggcaagacaaaattttgtgatagttatgtgagaattctacgtttccttcagatatatgcttctcactctccttccggacatttggtacagtcatgctgccaaggatcatgacctaccaatgacagtcttcactcaggtcttttgttttctaaatggtcctacgtccaaaattcagaattcgattaccttgtcgctaatactaaaactccaagttattttcttcagtgaaggtaattagtaacaaagtaatcaaatttttgaatgactaccagggcttagcgcccctccactctcacccctacacacacccagaggcaattgtattttaagagagaaagctttttttttatgacatacaagtcagagaattttagaggcatttgcctctaaaactttcactaacatcatttgaatcaagtttgaaagaattagtgaagtggcattgtgttataacctctactctgtttccacaataaaattattttcgtcactttacacaactttgttgcttcagagtgtataattctgtttcatgaattagttcacacactatgggaaaataggtaaatgttgtctgtatatatagtgtgtcagtataatgtggaagctgcattgactttattgtacacagttctccagtaagtgtgcaccagagagtaccagtaactgaatgtagagtgctggcacagtggaatgcagccagctgcgggggatgtggtacatgaggaatgtgctcatctgctcttttggccacgtgctccatcttagaggtgcttattgtgtggctttatccagtctttatgccccctcctctttttgctcaagtctgcattatagcagttgtgacctttccttatccctgttttctaatatgctactgttactgttccctgttcgttactggtttttttgtttttgtttttgcattgttctatagttactattgtatattggaaattagagatgctttctgtacactagtactcttattttgattattaattatgtttgttaacactaattacaaagttgcataagtattaagtagtctgcaccaaactgtttttgcgtaggaggtttttaagtgcaaaatttaaaagattgagtattttcagggacacatttatcacaagaaatcttataaaaatgtctttctcagtagaacacctgtatcaataaaccaaactttttgaatgttcatatctaattacagtgttttttgactaatttttatctatatacctgaaaaattcagtttaacatattaaacactacaggccaggcacagtggctcacgcctgtaatcccagcactttgggaggctgaggcgggcgaatcacgacgtcaagagatcgagaccaacctggccaacgtggtgaaaccccctctctactaaaaatacaaaaaaattagccagttgtggtggcgtgcatctgtagtcccagctacttgggaggctgaggtaggagaatcgtttgaacccgggaggcagaagttgcagtgagccgaaatggtgccactgcattccaacctggcaacagagtgagactccatgtcaaaaaacaacaacaaaaaacactccttggaaaaagtataggtacattctatataggattagtagactgtcagaatcacttgaagatgttccgaaagcatgacatatcaacaaaggacatgtgagtctagaatggtgatccctaggacaggccatattactgcatgcaaagcaagtctttgttaaccttatgacttgctcaggaagcaaagtgtttcatgtcctaataggatagtagtattttctgttatgttacattttaggaaattaggagttttgcattgtcttgtaagaaggtattttaaaaattttcaatgaaaataatgagaaataggtttccagtggttttatgcttttggggaagaggttactgatgtttagcaaaagatgcttatatattcttaatagaacaaacctgagaattagttcttaaagagttaaggcccataaaagaaactgtaagtgttggtactcttgtagctgggccacagatcagatccttagtatgataaaatttggtatttttgtgcttcaatttttggagaccattcaatgatcacttacattttatattgtctttcattgtgaaacatatattatagcagaggtgggagtcattaaaaattctacatgttgtacactacatgtataagggtatatttggcgcaatggaggtaggattgtctgtgtgcctctaaaattaacctctcaagtagttaatctgatgatgacaagtatgtcactttattgataaaagggagaattaagactattctcagtattatgttggtcagtacacaaaatatatcatttgtttttacctgtgaaatgaaatatctttcatcctattagccagaagggagaaagccttggcctgaaaataacacttatctcaaaaagttgggaaatgtggtccaactctgtgcccaggaaaagaggaaatagattttgatgactacatagtctctgcactgcttgccagctcataagtttacttctttgcactgtttgttttcattgctcaaaaactattttctgtcactctatgagatttagtctaacctgattcctggctgtggaaagcagtttctacttaagagccgatgagaaatggtatattcttctcaatcactcacagaatgactaccctggaacaaactaggaagacccaaaatgaaccagtttgattttaatacatacatagatgctgaaggagcctttttttaagaccttgccttcttagtctttggagaacagagtactgaaacaagaaaactcacagattagtagtatttctgtgtgttctacagtgaggttttaagagctgtattatgattaatcagtacatgacatattggttcatatttataattaaagctatacattaatagatatcttgattataaagaaagtttaaactcatgatcttattaagagttatacattgttgaaagaatgtaaaagcatgggtgaggtcattggtataggtaggtagttcattgaaaaaaataggtaagcattaaattttgtttgctgaatctaagtattagatactttaagagttgtatatcataaatgatattgagcctagaatgtttggctgttttacttttagaactttttgcaacagagtaaacatacatattatgaaaataaatgttctcttttttcctctgattttctagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccgggtaagttgggctgctagattaaaaacctaataatggggatatcatgatacagttcagtgaattcattttaaaagtgactgaaaaaaatgataccatatagcataggaacacatggacatttctgatcttatataagtattatacttttgttgttcctgtgcaagtttatagatgtgttctacaaagtatcggttgtattatataatggtcatgctatctttgaaaaagaatgggttttctaaatcttgaaaactaaatccaaagtttctttcattcagaagagaatagagtgttggacaaagaccagaacaagagaaatgtggagatacccaataataagtgtggatgtgcagtcttgaactgggagtaatggtacagtaaaaccataccataaaattataggtagtgtccaaaaaattccatcgtgtaaaattcagagttgcattattgtggacttgatgaagtgaccatggcagatcagtgcctgttgccagtaagagttgagtcctgctgggccacttagaatatccctgtgtggacggcactgcctggctgtggtagtttcttcccacttaaaaattcatagagacaactctgggtcttggaacttctggaaaacagcagctattccaaaaatctggggattagtggtgcctgtaacatcaagtccagggagattgagcagggaaatactggaaactcaggtactcatcctaacttcctggttttccagatggggttgccatttgttccttttctttcctccacaagataattttagtgtatctgtttaagaatataagagtggccagaagacagaaagcagtcgtcagtcacatttcaccaaaatttacatttgtctcaggtgcattatcgcagagctttattgtatttctagtattagctatccctgtcattgctgctacttttttatcatcttcaagtaaaatttaatgtcaacaaaaattaaggtagttaataatactatagtatactagtaaagttgtctgacacagacaactaggaaatgtgatttttactttaaaatgtattttagttttagaatgtctaaattttagttttagaattgtctaaattcgagaattcataaagctgtagcccggtggaaaaagttatttgacatctcagaaattaaaaattgcagagattttgtgtgttttttttttttttgttactggtttgttttttttttttaatgtcagatatgtttatagtgttggagaaagattctgcattatttaagcaactcaggtggttttctagcagagacaaaagggggctttaactgcctgaagtcattattattgcttgctttctggtaccctttttatttcacttacatagttttgagacacatgaataattaatacacttaatgactgatcataaggcagccagcaccttactttacatagtttagcagaatgatagggtattttataagaatgtgagtgatgttttatgaaaggtatgttgaggtctgtctatgactatccattgttggagcagaactgcaaatgccaaagctacatactttatgtttggcttcttgaggtgagatcccttcagctgtcttcatcaaaagctatgtctagcctttaaaaggcaatgaaattacactgtaagaatcattccagggttcaaaaaattgtctaccttccttatctgtctttcgctcagaagtctactcagtcttattttcctcagtcccccatttttttctctagtaaagcaagactaaaaacaaaagtttaagtacttggtatatactttattctatattttaaattctattctagaattttaaggaaacaactgcttttaaagccttaggtgttagtgtaaaattaactgtgagctctttaattatggtaacttgtttttagcaagttctgcttaattatatcaaaattagataatatagtgtggcctatcataaatcaggattcactagatagtatatgtacttgaatattgccagcccacaaaagcagttcacttcagaaatcatgtgtgttaggcagtttaataaactgtgaaaatagaaagagatgatgcttacagagaagcttccttaggggctctgaatgctcacactaagcagattttactcttaactttctttctcccttgctttttcttccttttcttttttttaaaaaaataacagaatgtgaaaagactagaaaaaccagcatcgaaataggtcttataagtaaaattgataacctgtgtaattttttgaagtaagtattcacagcaggtgatgattatgataaagccacatattttagtaacacatagtagaagttctataatagaaagtggtttaagattattctctcatatgaaagctcaaaagtgatgaatttttcaagatcttatttcccaaggatctctgctttttaagcctcattcttatatatcacagtgaacaaactagtggccacttttttatgacttgctaattttggaattgtatagcagggttgggtggaaaagaagaaatgagaaactttgtggtaagcttaatattgataaataaaattatgggatcctaggctttaagagctaaaagaaaccttagagttatccactccttagtttagaaatcaagcccagaaaatagtgacttcatatatgagactcttgtatgttttctgttgaggagtggtaacttgaattaataattgtggatttttttaaaaaagaaaaatatattcattgcttttatatattgtgaaattcaaaccagactagcagcttttttcctgaggtttttagagaaattgtctttgatgacaagagaagaattcatgttgatagtattttcactaatctctgattgtaggaaacactatcattttattactgctaagacagaaaaataccagcaattcattgtaagatgctatcatttgtatggtgtaatctgatttcagagataatgtgaaaaaagtgaattttagaatcaatagaatataagttgtctcttcacaaataccttagaactgcttaagaacacccccaccctacccgtttaacttactgccatagttcccctaggacttattttcagtattttttcgttattgttccttagaaaacagtacagtgataaacatgagcttagacttcacctttcatttgaatgacattcctaatgtgatgaggtttttacaaatatgtaaaaccatatttacaaatttacatttttaatataaatatgtaatttaatttaatatgtaaattaatttaatttaaatatgtaaatatttacatatttacaaatatgcaaatatttacatatttacaaatatgtaaatatttacatatttacaaatatgtaaatatttacatatttacaaatatgtaaatatacccatttgtgtgttcttttacaagtagataatttctcatttttcttagatgaagcacttccaatgatagtatgtattaagattttttaaattacttcttaaaggagaagatagaggttataagtttatggaagaatatagtattgccatcatgaggtccctcaaactgccttcacatctgaatgggaaaattgtttaaaaaaaataaataaataaataaataaaatccagggccctaacctctagaggtttagattagaagctgtattgtagagcctatgaatctgaacttctggtaatactccacaaatattttgataaagccagactccaatatttgggaagatggcctttggttcagctcaggtgggggtgtgtgtgcatgtgtgtttatgtgtgtatgtgtgcatatatagagagaaagagaaaggccattttgatatacaagtatgtttatgtgttttggaaactcagatttttaagaggatcatggggacagcaagcttggaatagaggatgatgatgatgtgaaacttggttccaattagtcagggagttctagcatttcacataaatatctaaaaaccaaaaggaatggctgagatgtggaattgaatagggaagttcaaaaataggaattagcacagataaaggtgattggcacagataaaaagccccactaccaggaggttctgcaaaatcccctctaaaagtcactggccctttttatctggtttctaatagcctagtctaacccagcttccagcattttcagctcccagcttccagcattggtgaacactcttactagtgaaacatgaacgtaattcttttatttattcattgactcacagagctttaaaaatgagattattctgtgctgcactaggtatgtttaattttgtcttacacatacactatattgtggttaacatgtaatttccattgttctttcttgtgatttattctaattgaaatcaaaaatgaaaccaagaaatatgcttggtttatgccctcaaagtaatttctcagaaactgaatgagagaatgaagcctatatatacatagatcctcagcattatataataatttaagacaatgaaacatcaaacttagtattacatgtgaggaaaatgaaatctaaaagagattccagcctacataaattaatggcagaattggaaatagaagtcaggattccaggtctcatggacgaacgtgattatgtgatttggtgttacccaaacatttaaactacatttgagatataacagacttttcttcatgtgtttatcgtgaacatgaaaaaattaatttggataatctggaaatctcaagcaatagtttaagcctcaagttgactgaagtagttgaagctagcctttctttctttttcctctttttttttttttttttttagacggagtcttgctctgtcacccaggctggagtgcagtggcgcaatcttggctcactgcaagccccgcctcccaggttcatgccattctcctgcctcagcctcccgagtagctgggactacaggcgcctgccaccatgcccagctaatttttttgtatttttagtagagatggggttcaccgtgttagccaggatggtctcaatctcctgacctcgtgatccgcccgcctcggcctcccagagtgccgggattacaggcatgagccaccacgcccggccttgaagctagcctttcttagaaatcccaggcattcttaagtattagaggtctcttttatctgattattgcttctacttaaatattcatggtattaaggaatttttttaaaaaattatgaattgatttgatgtaatagctcagaaaactataagattttaagtgataaggttttccttttgattcctgtaagtctagtaatatcatattttgatattaagatgtcatcctgctaggtattctgcaaatgctttgatatcaggtcagattttttttttaaaaaatgaactccctaggatttcatcatcatggccaattaaaaagttcagaaattaaaaatcattttatccagcaattgatgaaatcaagagtcttaaagaagaggaagttacgcagtgaagaggtagatatgattatatccaggatatttttgtttatttttccccagtaatctctgtctgttgctagtctccatgttaaataaatacaacatacactgtacttttaaatatacctaaaaattggcctggcgcggtggctcacacctgtaatcccagcactttgggaggccaaggcgggcagatcacaaggtcaggagaccgaaaccatcctggctaacacagtgaaaccccatctctactaaaaaatacaaaaaaaaatagccaggcgcggtggcaggcgcctgtagtcccagctactcgggaggctgaggcaggagaatggcatgaacctgggaggcggggcttgcagtgcgccaagattgcgccactgcactccagcctgggcgacagagcgagactccgtctcaaaaaaaaaaaaaaaacaaacaaaaaaaaccctaaaaatccacgtgtgtattgtaaataaattcaggcatgcatgcacgttctccttttcagaatgtttagacatagtcctccaaatttcaaatatcctaatgcatttaaaacaacttctgacttaaaaaatacagttttatggtgaggtttgggtttaaacaacaacatacaattttaaataatatagctattatgtaaagaatacacctttacagtttatttgttaaatcttttttaaattgaccgaacaattgatggaggttatttttaagaaaaagaattatcagtacagctgtttatgcaataatggcctttatcaaaatggttattgctaaattttatgtgttaataatttaaaataaaatagttttaaaggctatttatgtattctgtttttactattttatttattttaatttttgagacagggtcttaactctgtcacccaggcctggagtacagtggcacgatctctactgactgcagccttgacctcccacctcagcctcccaagtagctgggatgacaggcgcatgccaccaccccccactattttttatttatttatttatttatttatttatttatttattagttttttgtagaaacaaggtttcatcatgttgcccaggctgatctcaaactgctgggctcaggcaatctgcctgcattggcctcccaaagtggtgggattacaggtgtgagccacggcgcccagcctttattcttaagtggtacagtattttatgaaatcacttatgtgtaccagaatggtaatctaaaataggtaataacaaaatttttgcctacagaattcagttaccaactacttttcaaattattctttttacatatttatatttgaacacattctgactctgaaaaatgtatttgttgtaaaaaatggaaaaatatggaaaagaataaggaaaataaaaatcactcataatcctaaaaaaaaaatttttggtcgtcattgaagagagtgagtaaagcatagtagttaagagcttgtttcctggattagtatctctgttccacaacttactgtgacttactgtgacttgaagttatttcacctttcagtatcttggggtccttatctgtaaagtggagttaatgtttccttcctcataagggtgttgtgaagattaaattagttgctatgtgtgaaatgctacaaaagtgctatatattattgttgctgtattcatggaatatttttgttctataatgctatggtaggttttcagaaaatattttagaaccaataaaataacagcaacaacagctagtacttttaagcactaactttatgcagggaattaatctaagcagttcatacattaatttattactatattattatggttattttacagatgaaataagagaggtaaagttgtatgtttgcatttacaatagaacatattgcattgcattacattcttttggcaccaaggtggggagcatagaatagagatcagtggaagtggacagacatgtaatattttatggagaataaagataaatttgcttcagttatcatttgacatgtcttaggtagagctaagactagatagaacacagggcctctcaatcctgtagaaattcaccaccacatcctttaaaaggttttacattgataaggagtcagctctcatctatgaatgttagtggggaaaagctgtagaatgcacagtctaaataccagataaccttttttttccccactttggaatacattatatttgataaaagcaagtatacctttggatgtaaaggtaaatataaacactgacttacatctttaacacctagccagttgggaactggcataaggaaactcagaatggattacatggcaatatggaatgttggcctggtcccacatttgagctaagatgttaattctaattttagtctcctcagaattctgtatactccgtagcttgaaacaggtttccaactgaaactatttttttccccaaggttattttcacatctacatttgtttgactctagatcttggaactagtctatatcaagttcctttggggacacaaaattgcaaatctgttgttcagagagcaaaactattttaaaaggtaacactcgttctcatatattcttaataagccttcacaacttacagtatgttgaggaaactgaggcttaattgaatcgcgtgctaaagtttaaaggttaaaagttatggtaagtgagagttgggactagtaaggtgaattctgactgactcttggtctttccataacaaaacattgtctaaaagtaaactgtgtggcctaaaaatgattttgtaaatattagagatcggtgttgaacaacaacagacattatttaaagctaaagactaaattcacagaataggaccctaaagttgaagatacccagaagataatttcagctagttttgtcaaactctttgactgcaatccctagtattttacatagcatcccagtacatgcttatccatctgtacatctaattgaaacaaaagtttcacaaaacagtaggttccattactatgtggaatgcattctgattttcccattgctgattgcaaaccactaaatggattagactacccactaatgagttgcaggtgacaagtttgaaaaaacaatgctctcgggcaacctgacctgatgcgttaatttatgagattttattttttgtagacagactagattgtccaaatgagtattattactttaactactatagtgctgtgttacagttgtttcagtgattattgcatttttcaaaaccatttttaaaattctgtttgaaatagttggaataacctgaaaacatttaatatcctgttttggtaaattttcatctccaagattaaatttttttaaccagccaacagatgtttgaaggcaagttaggagatatgatggatactggatacagtatccatccatgtactagatacagtatccatccatgtactggatacagtcatactgttagcaaattttagatacataaagtaaaacaaacaaaatgcagtataaacagttaatattggaatctagtgttgattttatgattaatctcaatgaggttactgtatattcataattctataggtacacaaaaatatttggtaaaagatttatagttgagaaaatgctagacactaaagtttgttaacttacaatttaagaataagggctgggtgcagttgctcacgcctgtaatcccagcactttgagaggctgaggtgggcggatcatgaggtcaggagttcaagaccatcctggcaaacgtagtgaaaccccatctctactaaaaataaaaaatttagccaggcgtagtggcgcctgcctgtaatcccagctactcaggaggctgaggcaggagaattgcttgaacccaggaggcggaggttgcagtgagctgagatcgcgccactgcactccagcctaggcgacagagtgagatttcatgtcaaaaaaaaaaaaaaaaagagtaaggacatgagtaaggttatgtctcacctgtttttaaggaaatgtggatataagatgggttctagtccattaaaaggtggtaatttatactaagtcttactgtgagagaccataaactgctttagtattcagtgtatttttcttaattgaaatattttacttatgacttagtagatactaagacttaacccttgagtttctattctaataaaggactactaatgaacaattttgaggttagacctctactccattgtttttgctgaaatgatttagctgcttttccatgtcctgtgtagtccagacttaacacacaagtaataaaatcttaattaattgtatgttaatttcataacaaatcagtaaagttagctttttactatgctagtgtctgttttgtgtctgtctttttgattatctttaagactgaatctttgtcttcactggctttttatcagtttgctttctgtttccatttacatacaaaaagtcaaaaatttgtatttgtttcctaatcctactccttgtttttattttgtttttttcctgatactagcaatcatcttcttttcatgtttatcttttcaatcactagctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtaaatacattagtaatgtgattatggtgtcgtatcatcttttgagttagttatttgtttatcttactttgtaaatattttcagctatgaagagcagcaaaagaaggatttggtatggattacccagaatcacacatcatgactgaatttgtaggttttaggaactgatttgtatcactaatttattcaaattcttttatttcttagaaggaatattctaatgaaggaaattatctctttggtaaactgaattgaaagcactttagaatggtatattggaacagttggagggatttctttgctttttgttgtctaaaaccatcatcaaactcacggttttcctgacctgtgaacttcaaagaacaatggtttgaagagtattgagagactgtctcacaagtatgtcatgctcaaagttcagaaacactagctgatatcacattaattaggtttatttgctataagatttcttggggcttaatataggtagtgtttcccccaaactttttgaactccagaactctttttctgccctaacagaagagttgttattgaacacagtttgggaaaggctgatgggatttggaaatttgaaagtgaaggatcagaattttagttttttcccttttgtgataaagtagaacagggaaaagatgcagtcttttgggtagtctacttaacttcataattctgaactggttcagtttctactgtaaatataaccacttagtaactgagcttgcttacgtttaaaattgagtacatgacaattacaggaaaaggtttccactgaaggtaccatcagaattgtgaggagtgtgcatagaataatgtatgtcatttcccttcagctttgagatttgagctgttatagcctgttgattctaattgagttgacctttctgttactgttcttagtcacacacacacacacacacacacacacacacacacacacacacacgcatcccttatctataatctagctagtgttttattaataactaaaaagctatgccatttgtatgtagtttgttctaagtaaatcagagatacataagacgacgccctttttgagatagaaaattataaacttcataaagttcttaaatttggtaaaccttagctctagcttttgatgtatctagaaatgttaaaccttagctataaagcatacttgcattatatgcagaaatacttgtaagaaaaaacatagattaagcagttccagtaagataactgaagtgatggcagtagaagtatcaaaaaggagtattttaccaggaggttatggtgcttttctccctggaacatgagaaaatgtgcctaaaatggaacttcagagttatattctgattaacttatagcttgttgctcttggttccaaggaagggcatttgtgacattttattaaattcattaattttttagacacaccattgtcagcttgaacaaatttattaattgtaattatttgtcagctgttcttgatcctgttaataccatacttataactaaaagcatttccatggatgttgtaacttggcctgtaaaaaaaatgtttagatagaaaccatgaaactcaaatatgaattgtttaattttcaaaccattttgcattcagaaaatgtcctaagcttaattcatactcctagtgatcaaggaaacatgttaaagctccttatttttaaacttaaagtgacaatgacattttcaaagattttaaaattcttataaacaggttaaaatacttatatactgtataatttgatttctgatttctaagctctacttttctattggaaattacagatttttttcagacttaattcttaagatgttttcattgtttcacagtagcaactaaacatgtagtaaaatgatttaaattcaattaaaatttttttccttagtcatttaaaagggaagaaatcaatttttagtagtactcattccaaagattccaattttccttttttttaatcttttattttttggtggagggaggcaggatctggctctgaggcccaggctggagtgtagtggttctgtctcggctcactgcaacctccacctcccaggctgaagactcaaaccatccccatgcatcaccctctcaagtagctgagactataggcacatgctaccacacccagcttattttttgtgtttttgtagagatggggtttctccatcttgcccaggctggtcttgaactcctgagctcaagtgatctgcctgcctcagcctccccaaagtgctgggattacaggtgtgaactaccacaccgggccccaattttccaatgagtgatataaaaaaggcctccacgcaggcgcctgtagtcccaactactccggaggctgaggccagagaatggcatgaacccgggaggcggagcttgcactaagctgagtgctgctgcactccagcttgggcgacagagcgagaccgtctcaaaaaaaaaaaaaaaaaaaaaagcctccatgattggggcttgcatagtgaagaccatgtgaaattgaaagactacgaaactacttttcttttacgtattggcccataattaacatgtgtattgaatagctttgtttatctaagttcatcagatttatccaggtttatgtatttcagatcatctgattttattaggaaaatgctagaaaaatttcatggcaccattgtctaattttgaaaaaacgaacctttctttactgtgattaaaaattgttttttaggccaggtgtggtggctcacgcctgtaatcccagcactttgggaggctgaggctggcagatcacgaggtcaagaggttgagaccatcctggcaaacatggtgaaaccccttctctactaaaaatacaaaaattagctggcgtggtggtgcacacctgtagtcctagctactcaggaggctgaggcaggagaattgtttgaacctaggaggcagaggttgcagtgagctgagatcgtgccactccagcccaccctgggtaacagagcgagactccatctcaaggaaaaaaatgaaaaattgttttcaaaaatagtacgtgtggtacagatataagtaattatatttttataaatgaaacactttggaaatgtagccattttttgtttttttatgtttatttttcagctatgggtggataaagcatgaatataacttttcttatgtgttagtagaaaattagaaagcttgaatttaattaacgtatttttctacccgatgccaccaaattacttactactttattcctttggcttcataaaattacatatcaccattcaccccaatttatagcagatatatgtggacattgttttctcaagtgctaatataatagaaatcaatgttgcatgcctaattacatatattttaaatgttttatatgcataattattttaagtttatatttgtattattcatcagtccttaataaaatacaaaagtaatgtatttttaaaaatcatttcttataggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagtaagttctttgtcatttttttaattcagtctcttagattttatttaaatgcaaaaatttaatttatgtcaaaattttaaagtttttgtttagaatctttgttgatactcttatcaataagataaaaatgttttaatctgaccgaagtaccagaaacacttaaaaactcaaagggggacatttttatatattgctgtcagcacgaagcttttgtaagattgatttcatagagaagtgtttctaaacattttgtttgtgttttagtgaaatcttaagagataggtaaaaatcagagtagccctggctaagggtcttggtagttacaacgagtgtgcctgctcctaccacccccacccccaccttgagacaccacagaatttctcatagagcacagtgtgaattctattgctaaattggtggtatggggtttctcagcagagaatgggacatcacagtgactgacaatctttcttttataggttggaaactatttgggggactggagggatactgtctacactttttacaatttttattgataagatttttgttgtcttctaagaagagtgatataaattatttgttgtattttgtagttctatggtggcctcaatttaccatttctggttgctaggttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggtaataaatgtttttatgtcacattttgtctcttcattaacactttcaaagcatgtatgcttataatttttaaagaagtatctaatatagtctgtacaaaaaaaaaacaagtaactaagtttatgtaaatgctagagtccacttttctaaatcttggatataagttggtatgaaagcacacagttgggcactaaagccccttttagagaaagaggacatgaagcaggagatagttaatagctaagtgtggttgtagtataaagcaagaagcagggtgtttcttgtattaagctgtaagcaggaacctcatgattaaggtctttatcacagaacaaataaaaattacatttaatttacacatgtatatcctgtttgtgataaaaatacatttctgaaaagtatactttacgtcagatttgggtttctattgactaaaatgtgttcatcgggaatgggaataacccagaacataacaagcaaaaaattatgacaaatatatagtatacctttaagaaacatgtttatattgatataattttttgattaaatattatacacactaagggtacaaagcacattttccttttatgatttgatacagtagtttatgtgtcagtcagtacttccacatttttgctgaactggatacagtaggcagcttaccaaatattctatggtagaaaacttgggacttcctggtttgcttaaatcaaatatattgtactctcttaaaacggttggcatttataaatagatggatacatggtttaaatgtgtctgtttacatacctagttgagagaacctaaagaattttctgcgtctccagcatttatattcagttctgtttaatacattatcgaaattgacatttataagtatgacagttttgtgtatatggccttttcatagcttaatattggctgtaacagagaattgtgaaattgtaagaagtagttttctttgtaggtgtaaaattgaatttttaagaatattcttgacagttttatgtatatggccttttcatagcttaatattggctataacagagaattgtgaaattgttaagaagtaggtgtaaaattgaatttttaagaatattcttgaatgtttttttcttggaaaaattaaaaagctatgcagcccaataacttgtgttttgtttgcatagcatattataagaagttcttgtgattaatgttttctacaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccgggtaagaaagcaggtgtctgcaaaaagtcatgtatcgatttattgtttgtaatgatacagtagtatagcagataactaagacatattttcttgaatttgcagaatctagatttccaagcactagaagaaactacagaatatgacggtggctataccagggactctgttctgattaggtgaggtacttagttcttcagaggaagatttgattcaccaaaggggtgtgtgattttgcttcagacctttatctctaggtactaattcccaaataagcaaactcacaaattgtcatctatatacttagatttgtatttgtaatataatcaccatttttcagagctaatcttgtgatttatttcatgaatgaagtgttgttatatataagtctcatgtaatctcctgcatttggcgtatggattatctagtattcctcactggttagagtatgcttactgctggttagaagataattaaaataaggctaccatgtctgcaatttttcctttcttttgaactctgcatttgtgaactgttacatggcttcccaggatcaagcactttttgagtgaaatggtagtcttttatttaattcttaagataatatgtccagatacatactagtatttccattttacaccctaaaaaactaagccctgaattctcacagaaagatgtagaggttcccagttctatctgcttttaagcaaatgcccttactactctactgtctacttctgtgtactacatcatccaattctgaaagacataggcttccccatcccctgctaagactggttcaagtggcagctactgatggattgcagtgagaaggcatgcaaacacgtaccttcctggaagttgtctccaaaggctattgctctaagactcaagtatataaacactagaatgaatatcaactctatctagcaataaatgttatttttatattacagttgacccttgaacaacacagctgtgaacttcatgggccctctgacatgcagattttttttctcaactaagagcagattcagtattggtgggactcagaacctgcatatacagagggctgactttcatacatgccagtttcacagggccaactgcagaacttgagcgtgcatggattttggtatacacacgtggtcctggaaccaatccctgtcacatataccaagggatggctgtatgttactttatattcatttgttctgttattttataaggttgttcgtcgtggtatgtgggaattcaccagtatttcttctttctggtgcaccgttggtcatttctggcagcagtggtgaatgtatttactcttagcaacctctgtgctgctacctgttctgagtttcaaaggtgattcattaaagggttgggataacatggtgataggaaaaacccccctcatcagtcacaaggagtataacagcaatatctctgtaatatgattgatcatagatataatttctagtaggaaaaaaagtcatatcttgatgcatctctgagaatagttgaacatatcttgtgctattctttatagagaaattatctttgaaattaaagtcttaattttacttctagctttttataacaacataatccctacttggtatgtatcttaagatcatttttaaatgtatgatttgaagggcaaactagtgttatgtgaaaaatgacagataaagtagcttccaactcatcctcaagagttgatgatattctaaaccttttctaactaaattcagcttcttaattttctcaatataaatatgatgaaaatattaattcattaaatagtctacaagtattcggtagttgaagacttaaagtagtgcttgtaataacagaagagaaaaaagacattacaggcgtatctcactttattgcactttgcagatactgagtttttttggtggcaaccctgcatcaagcaggtctaccagcaccatttttccaacaaaatgtgctcacttcattagcatttttagcaatgattttaaattaagataatgtacttatttttagacaatgctgttacacacttgactacagtataatgtaaacgtaacttttataagcactgggaaacaaaaaaatttgtgtgactcactttactgccataatcactttatttgccatggtctgaaactgaaccggcagtatctctggggtatgcctgtatagatattttggttggtatttatttattgtatgcagaattcataaaaataaaaactgcgaggctgtttaatacatttcaactaaaagttgccagcatcattaatatgtaaaccactagaaataagattttgttaattttttgtttgtttgtttaaacagtcttgctctgtcactgaggctggagtgcagtggcgcagtctcagttcactgcaacctccgcttcctgggttcaagtgattctcctgcctcagcctcctgagtagctggggttacaggtgcacaccactacacctggctaatgtttgtatttttagtagagataagggttttgccatgttggccaggctggtctcaaactcctgacctctggtgatccgcccgcctcggcctcccaaagtgctgggattacaggcgtgagccaccatacctagcaaaatcaactctgaatctgaatcaaacctttgaaacaaaaattaccaatcaaaaaaacgtgcaatccctgctcctaactttgaaaaagtgacaaggaaggacatttggaaagatgtctctaagcagtcaacaagaaatgaaatcagggagagctttttcagcacctgaaaaaatacatgcaaaaacgcccgaggcgggcagatcaccggaagccaggagtttgagaccagcctggcaaacatgggggaaaccccgtctctactaaaaatacaaaaattagctgggtttggtggcaggcagctgtaatcccagctcctcgggaggctgaggcaggagaatcgcttgaacctgggaggcggaggttgcagtgagcctagatcgtgccactgcactccagcctgggcaacagagtgagattccatctcaaaaaaacgaaaaacagattcagagtttatttccttcccaaatacttcatagatgttggtttgtaggaataacttaaaagtctcactcgctttctgtcttttctagtaatttttatcgtggattctgattgcctagatctgctaattcattgagggttacaaaattgtgatactcgaattctttattcctccttcctttattacctgtaatacttcttaagaaaaactctttctcatcaactctatgactaccctaaattacagatccacatataggatctgtatacatgtatataaatatgtacatatcatatacgataatgcttaattctttcccattacttactaattttcaaaataatgtgattccctaactgctctctgaatgttatcaatgtggtggtaataatggtggtgtcgatgttgtgtggggtaaaatttgaatacagtgaaatacccagggcttaattgggttagtttttaaaaattacatttatgtaaccatatttcagtcaaggtgtagactatgtccatcagcacagagttcccatgttttcccttccagtgaatctccatcccccattagaacagacaataactgtcctaattactgtcactgtagcttacttttgtctgctctagaatttcttatgaatggaatcttatgtacttgtactgcatgaagactctttagattcatccatgttattgcattgatttatagtgccttttaaaaaattatagagtagtgttgcattatatgaatacgcaaagtgttttttttttacctgttggtggacagttggattgtttccagtttgggactattgtgaataaagctgtgtagtcttattgtggaaatgttttatttctcctgggcaaacatgtaggagtggagtttccaaagcatatagcaggtgcatatttaactaaaagtgtcttttcctcatttttaatattggatttttgtatttctattgactatagatattttcttaaaatatagcagtatgtcttatccttagttttgcttttttttggtttcagttatctttggtcagcagtggtccaaaaatatttattaagtagaaaattccatgaataattcagggtttttgatgtatgtgtgtgttggcgggtgcagggcagggagagttggttggttggttcgttggtttttttttttttaaagagacaaagtctctgtcatccaggctggagtgcagtagcatgactacagattgttataaactgaaactcctgggctcatgcaatcttcttacctcagtcctctgagtatctgggctcatgcaatcttcttacgtcagccctctgagtagctggtactacaggtgtgcagctaatttttagttggttatttttttgtagaaacagggtctcactatgttgcccaggctggtttcaaacccctggactctagcaattcttccacttcagccttccaaagtgctgggattacaggcgtgagccactgcacccagccaacaactcttaagttttaaattacatgctattctgagtaacatggtgaaatctcccacctttccacccagggcatgaattatccctttttccagtatatcgttgttgtctagactacccaccacttttagtctactggttatcagatcgactgttgcagtatcacatgcttgtgttcaagtaacccttattttacttaataatggccctaaagcacaagagcagtgatgttggaaattcgaatatatatgccaaagagaagctggaaagtgcttcctttaaggaaagaatttaaaaatcatatgttgaggttgctaaaatctatggtaaaaatgagtattgtatctatgaaattgtgaagaaggaaaaataaatcatgcatagtatatacagggttcagtactatttgcaatttcagacatcactggaggtcttggaacgtattccccatgggtaagtggggaatactgtatattctatataaaaggcccatttctgataagtagtttatgattattttctccaagttttcattttcttaagtgtcttctggtgagcagaaattattcattttttgaggtctaatttattttttcttttatggttttttatgtccttttatatctttgcctagcccaaagccacaaatatcttatgttttcttctaattgtgtaatataggtttaacttttatgtgtaaatctgtgatccaccaccaataaaattttgtgatgctgttagacagtggatgtcattcattttctaaatagttttatttatttgttccagtaccatttgttaaagagacttttctttccccattgaattgcctttgcacctttgttgaaaatcaatctgtgtgtgagccgtgtttctggattctccattctgttctattaatttgttttgtccttttgccaaaatgacactgttatggttactgcagcttcaaatcagatgctgtgagtcccacaactttttcattaggttgctttgacttttatttgcatataaatattagaatcagttaatttttacaaacaaaagggcaaagcctgcagagattttgatttgggttgccttgactggggcaggtcaatttggagagaattcacattttaacaatatattcttccaatccttgaacaccgtacgtctctccatttatttagatgtttgttagtttatctcagcactgttttatagtttttaatgttgaaatcatgtactctttggttaggaataaacttaaaatatgtttctaatgctattagtggtattttttaaatactgcttttcacttgtttattactatcttgaaataaaatcgatttttatatattgatcttatatcctataacctttctaaattgacttattctagtagttatttagtagatttcatagggttttctagtaaacagctatgtcatctacaaatcaagacaggtttctttctttccaatgtttatgttatttattactgttcctctattgcactggctagaacctttggtagaaggtattcttactttattcctcattttattgagagagactataatatttaccattaactgtgatgttacttgtaaacaaaaagtttgtatttctctgggattaaaaaccaagagtccatttggttttttactgttgagttttaagggttccttatgtattttctaggtatgttcttccttcattgaattgtttttttcacatacgttaaaaatcacttgagaatatttatataggtctgcttctgagatttatctgtttatcaggttttgtttcatttattttgaggctctatcatttggttcatacccatttagaattactatgtcttggtgattggtcctttatcattatataagttttttctgttttgcgattttctttgctctgaatctgatatgagtgtaaccatttgtttttttttaaattaatgtttgcatgatctgtcttttactatcattttactttcatcctgtgttgctgaatttgaaacgaatctcttgtaaacagcatatagttgtcattttaataaaaactgttagtctctgccttttaatttttttatttagaccatttatatttaaggtaattattgatatgttagggagaggtctgccattttattatttgttttctgtttctgtcttctgtttatggttatttatttattgccttccatgattacttgaacattttttaagatttttaagacccttgatttatttagtgtttgggttttgttttgtttgtttgtttgtttttgtttcgagacacagtctcgctctgttgcccaggctggagtgcagtggcacaatctcagctcactgcaacatcggcttcccaggttcaagcaatcctcccgcctcagcctcctgggtagctgggactacaggcacatgccaccacacctggctaatttttgtatttttattagagatgaggtttcaccatattggccaggctggtctcgaactcttgaccttgtgatctgtccacctcagcctcccaaagtgctggaattacaggcatgagccaccatgcctggccttctatgtgttttttatagttgttttcacggtcattgtgggtatcacattatagataatgtgacttaccaccattcactgccatcaataccattacatggaatgagatatggaaaccttaatttcagttgggtctctttccccacttttaaatagtattgttttgaggattaatgctattacagtttttgtatcagttatccaatatgacttatagaacttatcaagggagtgatgctctgttgtatgtacccatgtttctgctatttccattattttttccaagattccatcttttattacttccattagccagtcttcaaaggcagttctgctagtgagaaattctttttgtttttctctttctgaagatgactttattttccctttattcccaaagaatagttttatttgtgctatttggggttcgctcagcttcttgaagctgtaagtttgtgtcttttgccaaacatggaaaattttcaaccattatttcttcaggtgttctttcagcgtcactcttttctccattctaggcctctaatgatacaaatgttgagtcatttgttactgttccacatggacctgaggcatggttcatttttcagtctattttctctcttttgttcagatttctctcctttgtcatcttcactctactgttgagcccatctaatgattatttttctcctattgtattttttaggtctataatttccatttgcttttttttccttgtatcttgtttccttgctgagattttctattgtttcgtttttgaaacagtagaacttgtaattgcttgtcaaatcatttctctgatggcagcattaagatccttgccagataatttcagcatcttactcatcccagtgttggcgttttctttccttcatgtaattttcctggttcttggtatgacagataatttttggattgtgtctcggacattttagttattatatcaggagactctgtactttttaaaaattttaacttcagtagtaacctgttaaggtatagcatgtaggtactagcatatatttgtgagctgtggttccagtgaccatttttcagaggctttatggtgctgttttggtttgttgcttttatctcagtccactggggctcgcactggttcctagtagtgctgcttgaggggccttctccaggctgagctgcgcagagtgtgtttgggtgagggaagaggacccccactagattttcgtgggctggagagtgcttcctgagccttgtgcctgttgtggcaggctcccccttgctggtgttcctggcagtgctcctgaccacattgtgtccctgggctgtggaacacttgcaggaaaggtgaacactgccaggaccaattatagcagaattcctcctgccagtacacagcagcactgtggctctggaccagcaaggagagtctcaagcatgggtttgtttctttttgattttaagatttttcttctgccttcaggttctggtattattgttctgttttattcacctttatgtttcttctagtttagttttagtttctttgtatttgatgggatttttttccttgcatttctaattactccttagttgtgtcatgtcttcctttttttcagttaccctcattcagaaatttcccatttctaatgatttgttattttttaaattctttttagctcagtttgaaatttgtttacagttttcatgttttatttgcatattgtttcatgtactttgattttctgtagggacaccattctcatgtatatgcacatactttttctctaataattacactgtttcactctgttctgttatttatttatttgtgtgtgtgtgtgtgtgtgtgtgagtgaaatgagtttctcatacttggacgagggagagatgggccaggataacttttttcgctttttagggacgaggagaagtctgtgtttttgtaaaatggtcaaaaatatgcccttgtgctttctgaaactgccttctttactcctctatcccacacatatatttgaacttttttttggtttttattactgacctggtccattcggaagttattcccagaagtttttcctcggtacaaggtttgtttctggtgggggtttggtattcgggtgttttaagggcccagagcttactacatcatgatccctttgtaatcatctgcaaattagatcctgtagaactttttaaaactttaagccctgttttcagattggcctgtgctttccagtgggttggcagtgttctcagggctgtcagaaacccagtcagttaccttgcatctgcttccttactaaccagtcttgtagttgtcagagctttgtttctgtccaattatatgcaatggttcatggttttgttgtagataatgtctctgggtctttggttttctcattcagtttgctgtttttgtaggggaggttgggagggaataaaacaaaaaataataataataataactatgtcgttgccatcatcatctttccagaacctgctacctgcccttcacctgattttcgtatttatcagtatcattaccttaatctgttatctcattggggattgcaaaatggctgtttggggaaaaaaaatttccatctgcttgtcctacatatatacctggcattcttttttctcatcattgggtgccatttgattaccttgaaaaacagttctacagaaaaggcatgataaaatagatttaaggtttttagagtaaggagttaatgtaataattttaatagtgacaatttaattttgttttttggttggtttttgctgcctttttgaatattacgaactaatgaatgtttttgtatttaatacgtttcacagtcattactctaaatgatattcaaattgcttagctatcgtgtgcttttaaaatgaccctattagattttgagatttctaactaatctcgaacattcattctcaggcctggaataagttatttttccaataaattgttggagaatggcatttagaagtcagattttagatgcaaagtatggcctttgcttttgaacgttactgcttgtaggccttatcagaggacagaactaggaaaataatcttcctagtgtatcctgagaatcatcccacattattgcagaggtgttttccattttgttttgtatgactgaatactgctcctttgtgtggatgcagcatagcttatcaatatttatgggcatcgaggttatttttaaccttttgctgttagtactaatgtcacagtgaatcctcttaatgttcatgcattattccgtacatatgtaaagtatcattaagataaattgttagaagtgggattgttgcatcaaatggtagatgcatgtgtaattttgctagatactgccaaatcctccttcacagaaaaataacattttgcattcccaccagcagtttacaacagtatctcttttcttaaaactttccaaacagaatgtgttgttaaatgtggatgtttacttatttgccaattgagaaatggtattttactatagttacaatttttgagcttacctcaattatgaatgataatggacattttttcttgtagagctgtttatagttttatatagtcttttgctcattttgctgctcagctggtcgttttcttcacttttagaaactttatatgttaaggagattagtcctttaacgctggtaaattcgcaaatattttttcccagtttgtcatttgtcttttaaccttgcttataggatttttttgccaaatgaaaggttttatgtttatgtagttagctattaatctttttcgttattatgctttgatatgcatttctttagttatgtttattttattcattttctttgttttttaattttctagagtctgtcctcagtagaaattttagtcatactgcttcttctttggtcaccttataataaaattagcctttatttccaaagttaccttgcatatttcttcagtattttttagtattcttatttttatttaagatgtgctcatttctttctgaatttgtatttctcatcactggtggtctgtcttgtctgctgctgcattctccttttgtgttggaatgtggtgttactagtttcccattgtttatgactgaatttttctggtctacttttgtctaccggaaagttttgcttctcattcgccagactcttcccataataactttatttagacttccgaattctgtttttcttgtttatgtttctataacagatgttacttttttctggacctgttattactagacctcatggtggagcagggaggaattgtgtggctttaggggtcttggttgaagggatccctcttctgttgctttagtaagggcagaatcaccactttctgatttttcagctccatgtagcttaatgaagactctttttcattagctgcttccacccctttacagccgcttaagggacacttctgcttttgaaggtaccccccttcctttcatgcatggtgccttcctgagcctgccattgctggacccaagtacttttctgtagtccttcctttaaccaccctgttgccagtgctctaacctacactcagtagtgctctgttgaggggcaggtggtggagggggtggggtctggccttgggtgatccctaatcagtatgattccgcacaagccactcttcgatgtagctcctgctggaccctgctcatttccccacttctttggtatttggagtttattgttctgtctcctagttttactctagacctaagtcataggttttttttttattttcccctttttgatccgggtagatttttagagatgtgagtaggatgtgatcgatacagccaccacttttatgtggaaacctggaagcttcttcgtgttttcttcagctttctaaatatatgcattcttgtttaattcaaaatgttaatgtaacacacataacacttataggaatagagatgcattttgttccctgttttcattgtataaaatttttaaaggtttcgtttaaaagtccaaagaaacagctccgtaataatatagccagactggtcattgtgaccactgctgatctgccacttcctgcagggtctgtctctgttcttcggtggtgctctttggtctacctccttaggtgtagctggttcagttaagcttcagagccctcctgcagtgtgaggaggctttattaccacccactgcagggcagcttttctgctatgttggagggttctgtgctacagcagacttgttttctcttgtcagcctccccaaaaggagtatcatgaactggggtccgattcctctcctccttacctgagagatgctcctgcaagtttcatgtccagttttctgttgttgtgtgtttcttttttgttttctcttgctgactggtggatttgtaaaggggcagggaagggaagtggcaagagattggagaaagccaagaattctaataaaacttttaagttatttcagataaatggtgttgcagcaccaaaaatgccagcattttggtaataaaaatagtatgttgggatattagaatatcaaaaggtagaaaattgtaattactggtttgggatgtgtgatgttctctgctaacattctggaattttttttttttttttttttgagatgaaatttttttttttttttgagatggctcaaaatgtttcccaggctagagtgcaatggtgtgatctcagctcattgcagcctccgcctcccatgttcaagcaattcttctgccttggcctcccaagtagctgagattacaggcatctgccaccacgcccggctaatttttgtattttagtagagacagggtttcaccacgttggtcaggctgatcttgaacccctgacctcaggtgatccactggcctcagcctcccaaagtgctgggattacaggcatgagccaccacacctggctctggaaagaactttctaaataactattaaaatgcattttatattatataagcaactaaaaatacttttaattttttttttaattttttaatcttaacattatttagctataggccaggcgcggtggctcatgcctgcaatcccagcactttgggaggccaaggtgggcggatcacgaggtcaggagatcgagacccatcctggctaacatggtgaaatcctgcctctacttaaacgtacagaaaattagccaggcgtggtggcaggcacctatagtcccagccacttgggaggctgaggcaggacaatggcgcgaacccaggaggcggagcttgcaatgagccgagatcgcgccactgcactccagcctgagcgacagagcaagactctgtctcaaaaaaaaaaaaatttatttagctatgaactagtgtatatatgttttatagatgcctgattattcaaataaacttcaagattgcactttatgagcttgtatttcgttttttagaactcaaactaacataagtgtcattaaaacatagcttttattacatagattttaggaatgccacaggataaatcattctcttaaaataactgcaggtggcagaaatgtcagaataattctcctcctccatacacagcacatattacttgtttaaagtattctagttcatataaaaattgaacttttgtattactgctattaggtatgtagttgtttgcatttggggccagttggttggggcagggggtctttttttcttttgtccttaatctgtatcactttttcctcccaaagttgagttaaaggatgagtagaccaggagaataaaggagaaaggataaataaaatatatacccaaaggcacctggagttaatttttccaaatattcatttcagtctttttcaattcataggattttgtcttttgctcattactgactgcataatgtgattataccatagtttaaatagtcacttcctgttactacacacttgggttttctcaattttttactattgtagtactaatattttactatattgtaatctaatctaaatttttacgtattcagagctgttcaggataaatttgcttggaaatttttaaatcaccagaagtgatactatcctgataattaacttccaagttgtctcttaatatagttttaatgcaaatcataagcttatgttagtaccagtcataatgaatgccaaactgaaaccagtattgtattttttctcattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggtaggtaattattaacttgtgactgtatacctaccgaaaaccttgcattcctcgtcacatacatatgaactgtctttatagtttctgagcacattcgtgattttatatacaaatccccaaatcatattagacaattgagaaaatactttgctgtcattgtgtgaggaaacttttaagaaattgccctagttaaaaattattatggggctcacattggtttggaatcaaattagtgtgattcatttacttttttgattcccagcttgttaattgaaagccatataacatgatcatctatttagaatggttacattgaggctcggaagattatcatttgattgtgctagaatcctgttatcaaatcattttcttagtcatattgccagcagtgtttctaataagcatttaagagcacacactttgcagtcttgtaaaacaggtttgagtattttctccaccttagaggaagttacttgacttctcagtgacctaacctctaaagtgcatttactgatgtcctctctgtggttttgttgtggaaagatttagttaaatgaactgtaagaattcagtacctaaaatggtatctgttatgtagtaaaaactcaatggatacagtatcttatcatcgtcactagctttgagtaatttataggataaaggcaacttggtagttacacaacaaaaagtttatgatttgcattaatgtatagtttgcattgcagaccgtctcaactatatacaatctaaaaataggagcatttaattctaagtgtatttcccatgacttacagttttcctgtttttttccccttttctctatttaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaataaaacaaaaaaaaggaaggaaaaaaaaagaaaaaatttaaaaaattttaaaaatataacgagggataaatttttggtggtgatagtgtcccagtacaaaaaggctgtaagatagtcaaccacagtagtcacctatgtctgtgcctcccttctttattggggacatgtgggctggaacagcagatttcagctacatatatgaacaaatcctttattattattataattatttttttgcgtgaaagtgttacatattctttcacttgtatgtacagagaggtttttctgaatatttattttaagggttaaatcacttttgcttgtgtttattactgcttgaggttgagccttttgagtatttaaaaaatatataccaacagaactactctcccaaggaaaatattgccaccatttgtagaccacgtaaccttcaagtatgtgctacttttttgtccctgtatctaactcaaatcaggaactgtattttttttaatgatttgcttttgaaacttgaagtcttgaaaacagtgtgatgcaattactgctgttctagcccccaaagagttttctgtgcaaaatcttgagaatcaatcaataaagaaagatggaaggaagggagaaattggaatgttttaactgcagccctcagaactttagtaacagcacaacaaattaaaaacaaaaacaactcatgccacagtatgtcgtcttcatgtgtcttgcaatgaactgtttcagtagccaatcctctttcttagtatatgaaaggacagggatttttgttcttgttgttctcgttgttgttttaagtttactggggaaagtgcatttggccaaatgaaatggtagtcaagcctattgcaacaaagttaggaagtttgttgtttgtttattataaacaaaaagcatgtgaaagtgcacttaagatagagtttttattaattacttacttattacctagattttaaatagacaatccaaagtctccccttcgtgttgccatcatcttgttgaatcagccattttatcgaggcacgtgatcagtgttgcaacataatgaaaaagatggctactgtgccttgtgttacttaatcatacagtaagctgacctggaaatgaatgaaactattactcctaagaattacattgtatagccccacagattaaatttaattaattaattcaaaacatgttaaacgttactttcatgtactatggaaaagtacaagtaggtttacattactgatttccagaagtaagtagtttcccctttcctagtcttctgtgtatgtgatgttgttaatttcttttattgcattataaaataaaaggattatgtatttttaactaaggtgagacattgatatatccttttgctacaagctatagctaatgtgctgagcttgtgccttggtgattgattgattgattgactgattgttttaactgattactgtagatcaacctgatgatttgtttgtttgaaattggcaggaaaaatgcagctttcaaatcattggggggagaaaaaggatgtctttcaggattattttaattaatttttttcataattgagacagaactgtttgttatgtaccataatgctaaataaaactgtggcacttttcaccataatttaatttagtggaaaaagaagacaatgctttccatattgtgataaggtaacatggggtttttctgggccagcctttagaacactgttagggtacatacgctaccttgatgaaagggaccttcgtgcaactgtagtcatcttaaaggcttctcatccactgtgcttcttaatgtgtaattaaagtgaggagaaattaaatactctgagggcgttttatataataaattcgtgaagaaatgtgtgctcttcagttctcaagttttattattatggtatttattaaagttctacaattgtaataacgtatccatatgacaagttttaaagtggtaattgaaataggttatcagatatagagttgttcacatcaagtagacttttaacagagtcagaatgaacctacccttaaaattttagagaaaaaaaatcgtcggttgcacagaatagctgctctagcttgcttaattatgccgggcatgttgtcactcctcttacttttgctgccttttcattactatttaatggaatgtccctgaacaataaggaagagcaaaacatagacattttgactacagtggatacttcctctaccccaaatgttatgttataaaagtactttttttgcccaggtactctattatatattttggttttctttgaattagacctcaatctccaggaagctctggagggaaaaaaaggaaccataaactaaagtaactggttttccaaataaatgtaaacttttttaaccttttattattatagaacatttcaaacatacataaaacatgaaaacagcctgcagccaaacttttctggaccttggcagctccagcaaatgagctggtcatcctaaccctggtttcctgagacaactacttttggggatgcttgcttgtatagggagatgatagatagatagatacatatcaaaaagaaacttgaaaaaggcttcgaacagaacttgccgatgttactcatccctcctcggtacagacattctgttagaacagatggttgtaatggggcagcatcactccctattcccattaaaaacatgaccattctgagatttcaggccccttcgaggaatttttctcaagaattccatacaaactgtcagaggaagaaatgtcgtcccaagactagtccctgtgtagtcttgatccctttacctgcttactgtaattttttttctctgactttccgccagtgtctaagggccatctgtacccctcccacccttccatgagccactgtataatccaaaaacacaccttccatttgctccctcacttgtctccttttacctcaacagtgttagtgtgtaaagaattgcctggggagggtagagggttttacaagtacagagtcccaagtcttttctcccctccccgttagtgggttggcatttattaataggcactgcaagattttgatgcaggtgtggggtagatatcactgtgaaaggctactaatgcatggtagtttcaccgaaagtacagtcttcaaggatccttataatctcctcattgccaaaatcagtaacatctataaccttgacaacaccctagcttgggctgttgcttctctggctacttgtttttccatcccagtttacccagattttagatggatactcctccaattctgcttaaagtccttacctgttcttgcccagttccctctagtcaacctcagtcactcccaaagattcttccccaatcccatatcttttgctcagacctctgtctctgggatttctttgttacctcatagtcagtagtcctaaaaacaaaattcttcatcttgcatctctatcgaccgaccattctttgtttctttttaagaaatgtaaccttcaccccaatcatcaaaatctctcattcctcaattgtcacaggttgtcacttctttccccctaaacctctcccatcattccttccttctttatactgcccttacaggtcctgatcctctgactcacccggacattcttagcagctgcctaatttcactccctgactctcatctccctcttccaatccttttcattgtgctagaacactatcttgctaattttaatatcctctgcattcagtgcctccacagattgccccctcactgacctttccaatttggccttgtgttacacactttcctcgctgcattcctacccagcatcttcacttgtcatatttctcaaatactcttcatgaccagatatttaaagtacctcatccatgaagacttcatcaacccccaaagtgcctccaatgcttccgtggtagcacagcataatggggtacatttcttagttgaacagtgaggagtcactgggattcttacagagtgtcttagggtctcctcttgatactctttccacatttcagttaaggaattttcttacatctttttttcttctaataacctttgcctggagtgtaagtaaattcatgatagagactgtcatttttaaaaaaaaaaacaaaccattttgcctttcacatgttattacctccatctttctgttatcttgtcagtgttgccacctaaatgattgaaaatgaccgagcatgcagtagaatgtagtgaagtttcctatttcatccattttttgcataagtgcattggtatctgtatatccatctgcccaagcaccagttgttgaaaagactattatttccccatgaatggtcctggtacccttgtcaaagaacagttgaccataaatataagactttctggattctcagtcttactccattgccctatatgtctgtcgttataccagcaccgcactgtcttgatggctgtagcttggtagtaagtttagatttgggatgtgcaagtcctccattttttttttttttttccccaaaatcattttgtctgttttgggtcccttgcattgcatatgaatttttatatgatcattgtgtcaattcctgcaaaacagctgggatcttgatagggatttagttgaattggtagataaatttgagaaataactgctattttaatggtgttaggttttctgttccatgactgtgggatgtctttgcatttgcttaggtctttatttcagtgatgttttagagatttcagtgtgcaagtcttgacgtctttaatttttttctaagtattttactgatgccactttaattatatgattattttctaatttttatttttgtattcatcactggtgttagaaataaaattaatttttgtatattgattttgtatattgtagcttttctaaaattctttttttgtcacttagtattttttgcatatgagatcatgcatctgtgaacagaaatacttttacttcttcctttccagtctagattcctattatttcattttcttgccttagtgtgctggctagcacctccagtacaatattggatagaagtgacaagagcagacatctttttcttgttcctgattttaaggggaaagcattcagtctttcaccattgagtatgatgttagctagctgtgggtttgcttcaagttgaggaaattcttttattaaacctagtttactgagtgttttttaatcaaaaaaaaaaaaaggaggggctgtgggttttgttaaatgcttcttctgtatcaagattttcatgttgttttgtcctttattaatatgttgtattaatggacttttgtatattgaattaactttgcattcctgggataatctcagttgtccatgctacataaaccttgttctatgctgctggatttggtttgctagtattttgtttagaatgtttgcatctatattcataaggaatattggtctgtagttttcatattatatatctgtccagtttggggatgagagtcatactcctgacagaatgaattgggagctttccctgttcttaaacctactgaggtattttttaagtgaactacttggagaccttcacctgtgatcttcagatgctgggggacacagggatggtacctgactctgaaaaggctctcagggtaagacgtgtctgaactggcctgtggcagttaactaaagagtactcctaccaataatggagtcaagatgtatgtttcccataggccacgctggtcactgaaagagatgggctatgagccatcttgaaaactaccctgcacaaatggactgccggcctggagccaggcaccctgcagctgagtgccatataatgttgaagctgttgagggaggagaggttggaagctgccagcttagagaatctcagctgtggtttccagcagggtgtattccaggaaagaaagtcattccccaccctggctcaaagtaataattaacagtgactgggtaaggcaggatctttttttctaattccctcccctccagaaagggcagcaacagctcttgaatgggaaagaaggtggagggaggaaaagaaaaaccaattgccccaacactgctgtctttaagcttcccacccaaagacctcagtaagaggcagaattatgttttcaattaagagagagtttggcctcttggtgtttcaaaagtgtaggctttttaatacctagagaaactgatgatttgttgattaccaaaaagggaccagaaaagctacatagcctgagatttcatccccagaaaaacgaatccagagagcagacttgcaaaaaaaaaaaaaaaccgttctgtctctgcctctcccactacacatacacagtgagaaagagagtcttcatgatttggtaaattagacccatggaaagactcgggacatttaggatgatgatatttacgatgaggaagatttctatgggaaacttttttttaacttgtctctaatcctattccaactgaagtttgaatttttttcctttcattaacatttactgggcaaatacctattgaatgtttgccattttgcagtgtgccataggtttcaggctgattcagagctgctttgttgaatcaagaagtctgttacagggagacaatctagttagaccaggcataagtacaaaccactaagggtaaaagaagtatgtgaaagtacaatgtgggcagagaattcagatccaaaaatactgcagataggtaggtgatgaaggtcaagccgaatcttgacatacaggataggtgtatgtgagtgagttaggtggaaagtctagtatgtctgagtgtcaggctgtaagggattaagatgagattgggaaggcaggcaggtcagataatgagggccttatagaccataccaaggactttggacttttattttgcagacgttaagcaaagacataatcacttttcattttatatttattacaaactgaaagagcacaagtgcacatgtggtctttgcagttaagttattgccataattctaaaaaggcagataatctgaagatagaagggaggccacagtccaagacatgctagggactctgattgattcagtgctcatgcatgggaaagataagagtcaaagacaactcccacatttcaggttttgagagctggacggacagtgttgctgttaggtctatagagaagaggagtggattttgaagggaaaattattaatagtacaattgaatgatgttttagttgtgtctgagggatatccagggagagctgtcctaaggcaattaaaaatatgggtctagagaaaaacgtacacagaagataaaaatgtatgactccagcacatagaccatagctgaacctgtggctaggaaaattctcctggaaaaggcagagaggagagaaggacaaggatggaatgcatggaaatgctagcatttgagagttatacacggaacccacagtagaggcaaatgagaaaaagcaggtttgtttttgtctgctccagtagacagacaccaagctccatgagggcagagactttgtcttattcgtggctgtacctgcagtgcatagagcagactgtacatgtgctttgtggacccatgcagttactggggtgtctcagaaacttcccatcctcctccctactgtgtctttgcataaccctggctgggcctcccttttctcaatgtcaactctcactgtaactttttttttttttttttttttttttttgagacggagtctcgctcattgccctggctggagtgcagtggcggaatctcagctcactgcaagctccgcctcccgggttcacgccattctcctgcctcagcttcctgagtagctgggactacaggcgcccgccaccacgcccggctaatttttttgtatttttagtagagacggagtttcaccgtgttagccagaatggtctcaatctcctgacctcatgatctgcccacctcggccttccaaagtgctgggattataggcgtgagccaccgctcccagcctcttactgtaacttttaagaagggcatatatctacgtagtgtcatcaccataaagataaatcaaccaagtgaaaatattttgaaactttggaaatgtataaactccaaaatcatcatcagttacatcatcaattatgtccctaaagatcccatcactctgcctcacactggggtcttgcctacttctctgttccaaaagggcacagtagtggtatcaaatctgtttcttgaggcaaatgaagtaccttgctccaaaataatcacccacagcaggcagataacctccaagtgcttcaagctgagattgagcaggaggatcccagctcatcacaactgggtggtaaatatacacttgggcccaaatggaaggaaatgcattaccctacaatgcatacccaacatgcttgcatccaggccccaaatccagcaaaacaatgctattccccttcaacatgtgttaacggaactgagatatttttccaatttaaattgtgttccccaaagtcttcattattctctagtgcctattacaatctgtgtttttatgtttacttcatgactgcctagttccctcctgaaattttaagcctcgtgagagtaagaactatctgttttctttactccactgtcctgtacatacatacttgttgaatgaatgtatgaatgccaccgtgattgcaaagtccaagggaaatgatgttgattcactgcacactgtacaggagggtgtcaaccagacttccagatttctacagaaattaccagtaaagggtagtataagaatggttcctgcatgtgaaaaatgatcttggatggagctgttgtggcattcacctgctcctcatactctcctgccactgtgcagggacagcacaacccccgtgctgctttcttgagtgtagctcatttcagaaaagcagtaaaaggaaaacccacttgtttgctttatgttcctaagaaggtggtgagtaaatcaatcacgttatcctaagaagtgacttgaattttatgaaggctcctttgtttccattgcactcctttctacccatctctactcccacctccacccccaccctcaaatcatgcttataatttccattattcaattaagggtggcttttttctcatcacaaacatctgtgtgctttaaatagcttctacataaccgaagtggaagtagtcatgtagaacagcacatggccactccaggggacatgagaagcctctgcctaaaggtagttctcccttctgtcatctgatctcacacatggcactgcaaacctgcagttatttagccatgtgctttgtgacattgatttcacttccttgggttattcttaccgtctttaggaacttaaatatttaagaaataaacaaatactattcaaagagagctgtaccattattatatgcatttaagtgaaaaactaccacttcccatatatatttatctcatgaccacctaattgataaaaacatgttaactgttatcttatattgcctcagggaaaaagtctgaatgttgattggaggcactgcaggatagccattcgatacctgcttttgtgacaataaactaaagcattggattttggtggattagggataaaaatggcttgacagatcatcttgtccagccctcagtatatagatgagaagacttaggtctcaagctctgaagcgctttgctcaggatcgctggccatttcatatatgtaacaagctcccacttcatgctcatttcacagtagcgtgtcactttcaggtcagcttccttcagtgggcgttgtctttgcttaattaaatgtcaggaggtccccccagaaggcttgactcagggaaggagggcagaccctcccagttctctcactgactagctttcgtttgttggattgtttttactgataaaaaagttcagaaattagagccaggttcctgttcacgttccctacccaagcccctgaagaagcaaattggtgaatgttccccaaattcttttactctctagtatacagtaagcagtactgccaaaaagtttcccaaagtcatgttcctcttagatatttttccttgaaaactgttgtctggcatggagatccaacctgtgtggaaagttccagatgccctggtcctagcttcacctgccctgcttttgcttcctgttctcactgcttgtctccaggtttccatttgaaggcttgcagtgctactgtgcccttactgttaaatcctgccttgcttgccatgttttatagacaaggaaagcattttctaccagagctcaggagcagccttgaggtcaggggtaacgtttttcattggtaagtattcagtagttgctgaatgacaattgggtggaaaggggaggggtgccttagaaaaaatgtctgttctttgtggtaatgaaattgcttccaatttattttctgatctctttgagaactatttcctgtcgtcttcccgcaccatgcattctgatttctggggcattcagtgctccctcccttcctttattcaaggggctcttggaaaagaaatctctgaagaaaaaatgtgtgggcagtgggagacttctcattctctccaccaaatacttagccctttcattcatgagaaacccctgctttaggatggtgtggatctgagtcagcagggctgggttcatttgtatatactccaggttgtggaggttgttcctgggtcttggcctttcattttgggagtgctccacacttctgtctccatatactatgtatttaagaagcttgcctttcgatttcctgccagtggctggatcttggggatcactgcacaatgcttgtatgccccactggagaagtctctcatggcacctttcccctcccatggtggtggaatgctgtccacagaaagcttctcttcactgttcagagactctaaaaataagacctgaactgacttgctctgcctactgaaaggggacctccttatattcaccattgccctcacacccttttccttggagtctgtctttagggtcaccctgggagctgcctgggcctccaccttcttaaggaggcaactcctgagagctgggtagggactgtctttgtccagactgtctgaagagcatcttgttctttacctccctctcctgttcttgactattgccttcactgctgcctcgctctcgtctaccagggtggtattataaagtatgtccttggaggtgagctgtttgaccagctccttgttgcctagaaacctaagaaattgtcagaacaatccggcatgcatcagctctgacctttggagctttgactggcattatttataaaaggctttctgctctgttagccgagcattggcttcatatttctccatggactccagaaaactgggtttcttttttgcaagttctcttgttttcagttgagaacggtattgagtaggacttctctgtttccttgagttttatgttctctccaaatatggaatagcacttgcttccctgggtgccaacgctggccgaccagctctgaaagttgctctgcttgtggttcttccctgagatgctagttgggcctgggaatcttccatttctctgcccaccaggtgacctggcagtcagtgttctgtcaaagacctgtgagggtgcttagtgaactgtgggcactatgaccacagggtactattctgggtcccagtgtttgtttttaagaactctgtaggttgacaggacatgctgtctggtggccaagtgcttctgccaagagtatggggcctgggagacatgtttcctggcctgcttattgcaggcctagcttccacctaagagctatccgcttcccaaggggacatccaccatgtctctgtgataggagctgaatagtaggggcaggactgaaaggtctgtatgtttgcttttcagtcaggtacaggtgttggaggtttcttaaatttggtttcttttgatctccctggcaggcagtgatggggaagtttttagtaaggttattaacagagatagaggatttagagttaaaatttaatagggaaatttttctataggatggttgaaaatgatgctttgtatagttttattgtatgaacttttcagggatagaattagtcaatttgtagaaattgggccgcctctgttattgtgatatattagaactgtattgaatgatgagacttcccaaaagtttaaaacaaaaaaaggaggcagaagagaacgaggatataaatgaggacactctcgctgaggacacatcagctgttagcagtctgtctctgtgtaccgagctcaagaactgtgactttggttggaagttagaccttttctatccagcttcagacatggtaaccttcacataagctgctggatttgttctaccagaaatttagtctgttgtgtgactccagccactttgcaactcaagattattacttgttcaggtttctttcattaaatagctaaccatttaacaactattatcttcttagggtcgactgatggttacccatacagtatatacatcatccatatcctttcctctcctactatatctcactcctattttccagtccatcctcatctctcctcaagatcaacagaccccaacctcttttacttaaacgttgtattcctgaagtagtcctgccctttccttcagcctgcgaagccttctcaaagacttcttttccatgcatctagactctgcctggttattctccttccttcagagttatttctttgtcaaatgctgctaatgtataaaaacacaaagctttgatagttccttctgtcccaacggagccttacgcttggctgacatttagggtaaaggtggtatttttattagtaaaattaaatgatacagaagtgaattctaaaaatagttacataaatctaagagagttttcatgaattcaggttgccccaaagatttaggccatacctcttccatggaatggctgtcatgcagtttggatggttgacctcaaaattattcccagccccagaagtggagtaggtatattagtctaaggcagtcagggtaatgctatcttccatgccacagattggcttggaatggggataaccaaaagcactttgggcctgtgagatttgacagagtgtttactgtgagcctagggaaagaacatgcctgctctcatgtgagagtatgaagaaacactgcctaagcaaggaatcatgtggctccagtttttactgtcagcctttctaccaccagggagaccagccttgagatgatgcaggtgcatggacagctgtgtggagggatagaagaatccagtttttaaaccagatggctgagctatttcattaagtcaccccagaagtcttcaactaacagtatcttaactggtgaactcctgtgactgatacaggaaaattttacttcgtaacaaagtatgataattcctttgtgcaggaaggaggttttgtggaaatgatgttgattcactgcacactgtacaggagggtgtctctttgacaccaccaaccttaaatagtaggaaagccatcacagtgggttgggtggttggattgctttattgcaactaataaagggtcagttgtgcagttgttaactatctttcacttccctcagaactgtatctcctagcccagtgccctataggtcaaaaatgccagtttgttgtacagtcagcagagcccagtactatgctttaaaacattcggggataatcatcaaatatttaatcagaacagatgctgcctgtaaacaaccactgcagccaagaacagtctgcatctgaaacactgcctgcaactataaatatgcccatatgcttgaacaattttagtgttgtaaaaatcttgtttttataagcaattagtttcatctcctattatatgaggattaactttgcagtgatgtactgatgttaaaagagaaaagaatcaaaaaagccttggttgtggtgaaacccagcaaaatgtgggtctgcaccaagttccagatgaatatcaaacagcactgtcccagttggattaaggtgatgacacccctaccatggctacagtaccaggtcatttgattttccccagggtcataggagtgttagcccaggtgatcagctcaacaccccctgagatccatgaggtatatactttgaaatttcaattccattactactgactgcatctcaatcctatgttaatactttcttgagtaatattctgttgacctagttatccacgttggaaacttcaaaggcatcatcttcagctctttgtttctgactccctatccagttctccagttctgtatacaccatcccagaaatggtcctcgcatccagttaatctctgacatggactaataggcctttctacagcttcagtgtattctcttctctgcttctggaacccaggatttaggtgctacagtcatactatctttgacattccatctccctggagaatttggaaatggggaaataacattactcttaacaaagcaagattaacagtcctgtaaccagacccaaggcactgacccttctgtgaaagcctatatccaaccagctacaggaacctctacagagagactaaaaaaggccagccctggaggagccagtggtcaggagaacataggcataaagcctcctaaggatgtagcccatcaaccctgggagtgggttcctataaggaataggcacttcgggcaaatatggcccgatgacaccagccactgggatcttctgactctctcctgtataatgtgagttcatttgtttattgtgtttaaagtgaattctgcctccctaaaggctgagaacttcctttggctcagtgttcacttcgtttggcctctgacttccaactgatgcatcaagccaatgcgcaagaaaagttaataaataaaaataagatttattacagagccagctgggcactcatacatacatggaggagtccctgtgtcattggaacactgtagcttttctctacaacagtcccaggctaggcagctctttgtaggcccaagggaattgatgcataagtaaggcatggtcttccttgactacacatcttgtatacaaggtattaaggcccttggccacctgacttcggcccattttttcaatctcaactctaccctaaattccccacacaggtcattcattctccaatgaggttattctcctgatctctgtgtgggttcttatacataatggaattgtgtgtttttctcctgtttaaagtttggaacatcatgttagccatcattttattctctttgctttcactgtattgcactcacatactgtatgtggaatcagtgctacccattagacccatggctcatcagtgggaaatggtgtcagatctcacagagggcagcttcatggggctctaggttcttcaggcttccttcacagcctaagctccatatttaaatccagcattcaaccctaatcactgctgttcactagcatctgctacatattcttgctgacagacactgtgtttgctcttgaaagataacaaaatgcttgaagcttccctccaacgagtgataattcactgaagctcagaggcagcaggagctccagtctgctctgctgctctgaaattgtggaaagtaggaaaggtgtccagaggtcatatggcccaagtgatgacatatctgaagcacagagaggggatgggctaagagtcacctgtatccaaaattcaagttaacacctatcaagttgtgaaaagttactttgggacatcccaaagttacacagagagctagagctaggactcagacctggaacaccagacttcagtctctctgaactgcactgggttgtctctgctcaagccttattggagagagaactgacaaaaggcaaggctaaggagtgctctttgtggatgcagcaggagaagaaacacccttccaatgaggggtccacacagagttcaaatgtcccctttgttcatactgaacgtgggtatggttttcctcttttgctgtctgcacaggtggcatagaaggcagcacctgatccttcaggtctgggggaggagtccccagataatgcatagatcttttaaactcttcacgtcaaactgacatctgcttatgtagctcagctcaatgtgttagtcattttgtttcgtttattatcaaacacatcttctgaaaaatgagaagaggaagggaaaaaggagtctgggaaaggaccacactttctaccacccctgtaaacagtgagctttactctgcaatacaccaagtggaaggagacaggctatgagatgggcacagtaaagagccacctaagggaagtgggcagaactccagaagacttcctgaggaggcatttattctttatgaatctttgaaatgtttttttaaatcatctttgatttgacttttgctctaaaagtatcacaaggtaagactcagctgtgaaactaagcacatccgatagtcaaactatgaaaacttttacacgctagtggggcactgtgacaccactgatttccttaaactgagctcctttcaaggtgttttgacctaactggcctttgggtcggatatagcttttacatacttttggtaggtaactacgggttcctatttgtcagggaaagtgggtaagagttagctattccccacaaccttgatagcagcttataaagagagccagtgtcaaatcaatttgtgccacaggcattattttaaaagcatttcttagaaagcacacacaggagtgttcaggcaagaggtactgtaagccctttgctttttcggggcttacaacctctcaatttaaatgaaaggtttattacacaaggtattggtaacagtgaccagcaataggctctgctgtgttactactggcatcagtcaaaacatgttctgacctcgtttctgtatatgtttatatgtttagaggtcaagccctgtgtgtcctttctgagggccatgttaaaggcctcctcagattcctatactctattcttgcaaagctggagaggttctttagaagtattttagccgggatacgtatatcgaataccctcaacttcaccaatgcaagccagcttctaacatactaattagcaagccactgattttatcaataaaaacccatttctaacatttactgtggaattagcaaagtttctggtgtacattggatatgaaagctccagtaacagactcagaagaaatggagacctgtgaaatgacaaataatttagactaatcctcttaagttcaacaaagtacaagaaaatatggatagaaaaatgaaatttggaaaacaacatctgaacagaatgagaagtttaacaaatataaacaaaaacaatagaaattctaaagataaagaatacagtaactgaactgaaaatctaagtagaacgcttcaacagcagacttgatcaagcagaagaatcaatgagctcaaaggtaagacatttgaaattatccaatgagaaacaaaaacaaaaaagaatgcctataagaattacaggacacatcaagctagctaacttttgcataataggaggtccttaaggagaaaagaaagaaaaaggcctagaaagcatatttaaggaagtaatagctgaaaaattcccaaatctgagaaaagatgacaaaattcaggaacaggaagctgaggtcaccgatcagattcaacctaaagagttcaccaagacacatcacaaccaagttatcaaaaatcaaagacaaagaatactgaaagcagcaaagataagaaacatcacattcaaggtagccccaatacagattcagtggctttatcagcaaaagccttacaggccagtagagagtgggatggtatattcaaagtgctgaaggaaacaaagctgttaattaagaatactttacttggcaaagggagaaataaacacttttccagataaacaatgcctaagggagtttgtcactactaggcctgctctactggaattactaaaagggagttctttaggcttaaataaaagactgctagttaatgagaaacataaaagtaaaaaactaaatggtataagtaattcatagtcagaattctctagtattgtaaaggtggcaggtaaagcaatgttattcctattaggaggtttaaaagatgaaactattaaaaacaactgtagctacaataagttgtagctacaagaagtaattgtaactgtagctacaataagagatacaaatttaaaaaagtgtaaactttgacatcaaaatcacgaaaggcgggggggttgtgaaagcgtagagtttttgtatgcaactaaaggcaagtttttatcgcttaaagtagcctaaggataaaatgttttaagttagccttacagcaaatacaatggtaatcgcatatactataaaagaaaggattcaaagcacaccaccacagaaaaccaccaaaccacaaaggaaggtagcaatagggaaaaaagaaacaagggacctacaaaacaatcagaaaacaaattacaaaatgacactagcagtttcttaccaatagttaccttgaatgtaaacggattagattctccaacaaaaagacatagaatgacagaatggatttttttttttttttaagtcacaaccacatactgcctataagagactcacttttttttctttttctttttctttttttgagacggagttttgctattgtcaaggctggagtgcaatggcatgatctcagctcaccgcaacctccacctcccgggtttaagcaattctcctgcctcagcctctcgagtagctgggattacaagggcccacaaccacgcctggctaattttgtatttttaatagagacaggttttctccatgttggtcaggctggtctctaactcccaacctctggtgatcccgccgccttggcctcccaaagtgctgggattacaggagtgagccactgcgcccggcaagagattcacttttctactaaacatacacatagactgaaagtgaagggatggaaaaagatattctatataaatggaaattgagagcaggggtagctatacttctatcagacaaaatagactttaagtcaatactataaaaagaaagaagtttgttatataatgataaagggattaattcaccaagaagacataacaattgtaaaagtatatgcacccaacattgacccacctaaatacataaagcaatttttaaatgatgtgaagagggagataggctgcaatactataatacgggacctcaatactctacttttcaccatgggcagattgatcacctagacagaaaatcaataaagaaacaatggatttcaattacactttagaccaaacggacttaacaggcatatatgaaacattccatccagaagcagcaaaatacattttgttctcaaatgcccatggaacattctccaggatatatgttagtccacaaaactaatcttaagtttaagtggacagaaatcatatcaaatcccttttctgattacaatggcgtgaaactagaaatcaagaacaagaatcaattctggtggaaaatacagcaatatgtggaaattaaacaatatgctcccaaacaactaatagttcacagaagaagtcaaaaagtaaataaaaaattaccttgagacaaatgaaaatggaaacaaaacttatgtgatgcagcaaaatcagtcctaatggaaatttatagcgatacaaacctacgccaataaagaagaaggatcacaatctcacttttcacctaaaggaactaggaaaagaacagactaagcccgaaagttagcataaagaagtaagtaacaaagatcacagttgaaataaagactggaaaaaaatgaacaaaactaagagttgctaagagttggttttttttttttttgaaagataaaatcaacaaattcttacctagactaggagaaaaaaagagtactcaaataagatcagaaatgaaagaggagacatttactactgatatcaccaaaatacaaaggattataagaaactactatgaagttatgcaccatcaaattggatgacctaaagaaatgaataaattcctagacatatacaacctacaaagagtgaatcatgaagaaacaaatctgaataagccaataacaagaaaagagattgagccagtaataaaaagtatcccaccaaaggaaagcccaggattaggcagcttcactactgaattttaccagacatttaaagaactaataaaaattctcaaacttttccaaaacaatgaagcagagggaatacttctaaattcattttacaagggtagtgttacactgttattaaagccagagaaagacactttaaggacagaaacttacaaagtaatatccctgatgaacatggatgcaaaaatcctcaacaatatattagcaaactggattcacgaatacaaagaatgattcaccatgatgaagtgggatttatccttaggatgcaagggtggttcaacatatgcacatcaaaaagtgtgatataccacataacaaaatggagggtgtaaatcaaatcatatgatcatctcaatggatgcagaaaaagcatttgacaaaattcaacctggtttcatggtaaaaactctcaacagattaggtatggaggccatgtacgaccctcatcactttttgacatagtactagaagtcctaaccagagcagttggacaagagaaagaaaaaaaaggcatcctaataggaaaggaataggttaaatcatctgtaattactgatgacataaccatatacaaaaaactgtaatgtctccaccagaacaaaactaaaacctggtagaactgatacacaaactcagtaaagttccgtaatacaaaatcatcttacagaaatcagtagtgtttccatctactattaatgagctatctgaaaaggaaattatgaaaatctcataatagcaacaaatattaaatactgaggtgtaaatttaaggaggtaaaagatctgcatactgaaaagtataaaaggacagggtcttcaataaatggtgttgggaaaactggatatccacatgcaaaagaattaaattagacccttatttacaccatataaaaaaatagatttaaaacttaacatataagacctaaaactgtacacctactgaagaaaacatagggggaaatctccacagcattggtcttgggaatgatttttttggatatgactgcaaaagcatggcaaaaatagacaaatgg